(19) United States
(12) Patent Application Publication
(10) Pub. No.: US 2026/0231998 A1
(43) Pub. Date: Aug. 13, 2026

(54) COMPOSITIONS FOR MODULATING GUT MICROFLORA POPULATIONS, TREATMENT OF DYSBIOSIS AND DISEASE PREVENTION, AND METHODS FOR MAKING AND USING SAME

(71) Applicant: PERSEPHONE BIOSCIENCES, INC., San Diego, CA (US)

(72) Inventors: Stephanie J. CULLER, Del Mar, CA (US); Robert J. HASELBECK, San Diego, CA (US); Stephen VAN DIEN, Fallbrook, CA (US); John B. Jarman, Oceanside, CA (US); Hirokazu Sato, San Diego, CA (US); Sean Stromberg, Encinitas, CA (US); Pedro J. Torres, Carlsbad, CA (US)

(21) Appl. No.: 19/156,823

(22) PCT Filed: Feb. 27, 2024
(86) PCT No.: PCT/US2024/017540

§ 371 Date: Aug. 14, 2025

Related U.S. Application Data
(60) Provisional application No. 63/448,752, filed on Feb. 28, 2023.
(60) Provisional application No. 63/545,116, filed on Oct. 20, 2023.

(51) Int. Cl. A23L 33/135; A23L 33/00

(52) U.S. Cl. CPC A23L 33/135; A23L 33/40

(57) ABSTRACT

In alternative embodiments, provided are compositions, including products of manufacture and kits, and methods, comprising combinations or formulations of microbes, such as non-pathogenic, live bacteria and/or bacterial spores, for example as probiotics, for the control, amelioration, prevention, and treatment of a disease or condition, for example, a dysbiosis, or for augmenting the health or ability to thrive in an individual. In alternative embodiments, provided are compositions or formulations, including products of manufacture and kits, and methods, comprising at least one non-pathogenic, live bacteria and/or bacterial spore and at least one probiotic, or synbiotic. In alternative embodiment, these non-pathogenic, live bacteria and/or bacterial spores (and optionally also a probiotic) are administered to an individual in need thereof, thereby resulting in a modification or modulation of the individual's gut microfloral population(s).

### RELATED APPLICATIONS

This Patent Convention Treaty (PCT) International Application claims the benefit of priority under 35 U.S.C. § 119 (e) of U.S. Provisional Patent Application Ser. No. (USSN) 63/448,752, filed Feb. 28, 2023, and U.S. Ser. No. 63/545,116, filed Oct. 20, 2023. The aforementioned applications are expressly incorporated herein by reference in their entirety and for all purposes. All publications, patents, patent applications, and GenBank and NCBI RefSeq assembly sequences and sequence references cited herein are hereby expressly incorporated by reference for all purposes.

### REFERENCE TO ELECTRONIC SEQUENCE LISTING

The application contains a Sequence Listing which has been submitted electronically in XML format and is hereby incorporated by reference in its entirety. Said .XML copy, created on Feb. 27, 2024, is named “6411.154262PCT.xml” and is 346,666 bytes in size. The sequence listing contained in this .XML file is part of the specification and is hereby incorporated by reference herein in its entirety.

### TECHNICAL FIELD

This invention generally relates to microbiology, medicine and pharmacology. In alternative embodiments, provided are compositions or formulations, including products of manufacture and kits, and methods, comprising combinations or mixes (or consortium) of microbes, such as non-pathogenic, live bacteria and/or bacterial spores, for example as probiotics, for the control, amelioration, prevention, and treatment of a disease or condition, for example, a dysbiosis, or for augmenting the health or ability to thrive in an individual. In alternative embodiments, provided are compositions or formulations, including products of manufacture and kits, and methods, comprising at least one non-pathogenic, live bacteria and/or bacterial spore and at least one probiotic. In alternative embodiment, these non-pathogenic, live bacteria and/or bacterial spores (and optionally also a probiotic) are administered to an individual in need thereof, thereby resulting in a modification or modulation of the individual's gut microfloral population(s).

### BACKGROUND

Vaginally born infants inherit their gut microbiome predominantly from the mother during passage through the birth canal and then via breastfeeding, a process termed vertical transmission. This includes microbes such as Bifidobacterium species, bacteria that modulate the infant immune system, help prevent the invasion of pathogens by acidifying the gut environment, and act as keystone strains that support other commensal bacterial species.

Conversely, birth by Cesarean section (C-section) bypasses passage through the birth canal, thereby blocking inheritance of Bifidobacterium species and other important commensals, allowing dominance by inflammatory microbes such as Enterococcus, Enterobacter, Clostridial and Klebsiella species. This microbial dysbiosis leads to chronic inflammation that can cause asthma, environmental allergies, childhood obesity, immune disorders such as type 1 diabetes (TID) 0, inflammatory bowel disease and a wide range of cancers. C-section delivery, along with growing predominance of formula feeding over breastfeeding contribute to the significant loss of B. infantis and other important Bifidobacterium species from the general population. Moreover, mothers who did not inherit Bifidobacterium species as infants are not capable of passing them on to their own offspring.

Dysbiotic infants tend to grow up to be dysbiotic adults, leading to a greater incidence of inflammatory diseases such as cancer. Moreover, inflammatory conditions in the gut can cause high failure rates (greater than 50%) of antibody-based checkpoint-inhibitor anticancer immunotherapies, that block inhibitory signals of T-cells to potentiate their ability to recognize and kill cancer cells. Examples of important T-cell regulatory/inhibitory functions and the checkpoint inhibiters that target them include cytotoxic T-lymphocyte-associated protein 4 (CTLA-4, optionally ipilimumab, or YERVOY®), the programmed cell death protein 1 (PD-1, optionally pembrolizumab or KEYTRUDA®, nivolumab or OPDIVO®), and its ligand (PD-L1, optionally atezolizumab or TECENTRIQ®, avelumab or BAVENCIO®, and durvalumab or IMFINZI®). The likelihood of response or non-response to checkpoint inhibitors is directly correlated to the state of the gut microbiome and its contribution to immunological function of the gastrointestinal tract as it was posited that healthier anti-inflammatory gut microbiome better primed T-cells to respond to activation by checkpoint inhibition, while the chronic inflammatory state brought on by a dysbiotic microbiota led to T-cell exhaustion and checkpoint inhibitor ineffectiveness.

The negative impacts of both infant and adult microbiome dysbiosis highlight the need and opportunity to ameliorate and repair deleterious inflammatory responses by reintroduction of key commensal microbes that can help restore the proper modulatory immunological effects of the gut microbiome In both infant and adult cases, dysbiosis is at least in part manifested by loss of intestinal wall integrity due to degradation of the intestinal epithelium, either by the toxic effects of invasive pathogens and/or by the loss of supportive commensal short chain fatty acid (SCFA) producing bacterial species. Probiotic microbes such as Bifidobacterium have been shown to help re-tighten and restore the integrity of the gut epithelium by stimulation of toll-like receptors that act to increase the formation of tight junctions between gut epithelial cells. Moreover, probiotic Bifidobacterium can help improve epithelial cell survival and health by supporting beneficial SCFA-producing microbes such as Faecalibacteria, Anaerostipes, Eubacterium, and Roseburia species. There is clearly a need to develop new, effective microbiome restorative therapies to address these conditions and pathologies.

### SUMMARY

In alternative embodiments, provided are methods for:
controlling, ameliorating, lessoning or preventing the symptoms of or the mortality of a dysbiosis or an infection in an individual in need thereof,
   wherein optionally the infection is a bacterial infection or a viral infection,
   wherein optionally the dysbiosis causes or exacerbates a Failure to Thrive (FTT) of the individual, and optionally the dysbiosis is in an infant, a child, an expectant mother or a mother (material dysbiosis), and optionally the infant is between 0 and 36 months old,
   and optionally the dysbiosis can be the presence of a pathogenic bacteria, or a bacterium or mix of bacteria not normally present in the microbiome of the individual, the infant or the child,
   and optionally a high level of pathogenic bacteria, or bacterium or mix of bacteria not normally present in the microbiome, is present in the dysbiosis,
   or the dysbiosis can be caused by a high level of antibiotic resistance, or a metabolic balance that skews away from that of a healthy population, or an immunological state that skews away from that of a healthy population, or a loss of metabolic function associated with a healthy population, or an increase in bacteria associated with adverse events for a mother and her child,
modulating the microbiome of an individual,
   wherein optionally the dysbiosis treated or condition treated or ameliorated comprises a dysbiosis caused or exacerbated by: premature birth, extended stay in the neonatal intensive care unit, drug or antibiotic treatment, drug abuse by expectant mother, nutritional or environmental stress experienced by expectant mother, drug or antibiotic treatment of the mother prior to birth or after birth, birth via cesarean section, formula or nutritional supplement feeding, and/or known dysbiosis of the mother,
   wherein optionally the individual is a human, and optional the human is a human child or a human infant, and optionally the infant is between 0 and 36 months old, or 1 week and 30 months old,
   and optionally the microbiome of the individual is modulated to positively affect the growth, thriving or health of the individual (or increases the ability of the individual to thrive), or to enhance the efficacy of a treatment in an individual in need thereof, wherein optionally the treatment is a drug treatment, or a treatment for disease or a condition, wherein optionally the disease is cancer, a genetic disease, a mental or neurological disease, or an autoimmune disease,
treating, ameliorating, lessoning the symptoms or severity of, or preventing a disease or condition caused by a dysbiosis in an individual in need thereof, or treating, ameliorating, or lessoning or preventing a disease or condition whose treatment can be augmented by administration of a biotherapeutic (also called probiotic) as provided herein,
   wherein optionally the disease or condition is a Failure to Thrive (FTT), or a condition in the individual (for example, infant or child) where the individual has decelerated or arrested physical growth, for example, when height and weight measurements fall below the third or fifth percentile, or a downward change in growth across two major growth percentiles,
   wherein optionally the disease or condition is an infection, a cancer or an autoimmune disease, a hereditary or genetic disease, or a neurological condition,
   the method comprising:
(a) administering or having administered to an individual in need thereof a composition or formulation comprising:
   (i) at least two different species or genera (or types) of non-pathogenic bacteria (also called probiotics) and/or non-pathogenic bacterial spore, or
   (ii) at least one non-pathogenic, live bacteria and/or non-pathogenic bacterial spore and at least one probiotic (also called a synbiotic, or combination of a probiotic and a prebiotic),
wherein each of the non-pathogenic bacteria comprise (or are in the form of) a plurality of non-pathogenic colony forming live bacteria, a plurality of non-pathogenic germinable bacterial spores, or a combination or mix thereof; or,
(b)(i) providing a composition or formulation comprising:
   (1) at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable bacterial spores, or a combination thereof, or
   (2) at least one non-pathogenic, live bacteria and/or non-pathogenic bacterial spore and at least one probiotic (also called a synbiotic, or combination of a probiotic and a prebiotic),
wherein optionally the at least two different species or genera (or types) of non-pathogenic bacteria of (b)(i)(1) or the at least one non-pathogenic, live bacteria and/or non-pathogenic bacterial spore of (b)(i)(2), is genetically engineered to comprise or express a new or heterologous trait or phenotype; and
(ii) administering or having administered to an individual in need thereof the composition or formulation;
wherein optionally the composition or formulation comprises one, or a or any combination or mix (or consortium) of: one or at least two different species or genera of non-pathogenic, live bacteria (or spore thereof if the bacteria is spore forming) as described Table 1 or Table 4, or live biotherapeutic (also called probiotic) compositions or combinations of bacteria as set forth in Table 2 or Table 30, or the at least one non-pathogenic, live bacteria and/or non-pathogenic bacterial spore and at least one probiotic (or synbiotic) comprises a combination as set forth in Table 8 or Table 32,
and optionally at least one of the bacteria in the synbiotic as provided herein, or in a combination, mix (or consortium) as provided herein, is a Bifidobacterium or a Bacillus species, optionally a Bifidobacterium infantis species, and optionally the different species or genera (or types) of non-pathogenic, live bacteria are present in approximately equal amounts, or each of the different species or genera (or types) of non-pathogenic, live bacteria or non-pathogenic germinable bacterial spores represent at least about 1%, 5%, 10%, 20%, 30%, 40%, or 50% or more, or between about 1% and 75%, of the total amount of non-pathogenic, live bacteria and non-pathogenic germinable bacterial spores in the formulation, and optionally only or substantially only non-pathogenic, live bacteria are present in the formulation, or only or substantially only non-pathogenic germinable bacterial spores are present in the formulation, or approximately equal amounts of non-pathogenic, live bacteria and non-pathogenic germinable bacterial spores are present in the formulation.

In alternative embodiments of compositions as provided herein, or a composition, formulation or pharmaceutical formulation used in a method as provided herein:
wherein the composition or formulation further comprises at least one prebiotic (for example, as in a synbiotic as set forth in Table 8 or Table 32), a nutrient, a metabolite or a drug, and optionally the drug comprises an antibiotic,
or optionally the method further comprises administration of a prebiotic, synbiotic (for example, as in a synbiotic as set forth in Table 8 or Table 32), a nutrient, a metabolite or a drug, and optionally the drug comprises an antibiotic,
and optionally at least one dose of the prebiotic, synbiotic (for example, as in a synbiotic as set forth in Table 8 or Table 32), nutrient, metabolite or drug is administered before a first administration of the formulation, mix or consortia of bacteria, optionally at least one dose of the antibiotic is administered one day or two days, or more, before a first administration of the formulation,
the composition, formulation or pharmaceutical formulation comprises an inner core surrounded by an (or at least one) outer layer of polymeric material enveloping the inner core, wherein the non-pathogenic bacteria or the non-pathogenic germinable bacterial spores, or prebiotic, are substantially in the inner core, and optionally the non-pathogenic bacteria or the non-pathogenic germinable bacterial spores, or prebiotic are in the (or an) outer layer,
and optionally the polymeric material comprises a natural polymeric material;
the composition, formulation or pharmaceutical formulation comprises, or further comprises, a live biotherapeutics (also called probiotics) and at least one prebiotic (for example, as listed in Table 3), synbiotic (for example, a combination of probiotic and prebiotic as set forth in Table 8 or Table 32), or drug, which optionally can be prepared by mixing the two components together.

In alternative embodiments, harvested and/or dried activated microbial cells can be combined with at least one prebiotic or drug, such as a powdered or lyophilized form of a prebiotic, synbiotic (for example, a combination of probiotic and prebiotic as set forth in Table 8 or Table 32), or drug. The harvested and/or dried microbial cells and the powdered form of the prebiotic or synbiotic can be in a single dose packet, which can contain from about 1 million to about 100 billion colony forming unit (cfu) of bacteria and, optionally, from about 0.1 gram (g) to about 20 g of prebiotic or synbiotic, or between about 0.1 mg to 1 gram of drug.
the composition, formulation or pharmaceutical formulation comprises, or further comprises, a nutrient designed to produce metabolic benefit, such as tryptophan, or a secondary metabolite. Any composition, formulation or pharmaceutical formulation as provided herein can further comprise a secondary metabolite. The secondary metabolite can be a short chain fatty acid, such as acetate, lactate, or combinations thereof.
the composition, formulation or pharmaceutical formulation comprises, or further comprises, a stabilizer, such as a flow agent. Flow agents may include starch, silicon dioxide, tricalcium phosphate, powdered cellulose, magnesium stearate, sodium bicarbonate, sodium ferrocyanide, potassium ferrocyanide, calcium ferrocyanide, bone phosphate, sodium silicate, calcium silicate, magnesium trisilicate, sodium aluminosilicate, potassium aluminum silicate, calcium aluminosilicate, bentonite, aluminum silicate, stearic acid, and polydimethylsiloxane. The stabilizer can be a milk protein or another suitable pharmaceutical grade or infant formula grade diluent (for example, lactose). The milk protein can comprise a protein fraction of nonfat dry milk.
the composition, formulation or pharmaceutical formulation comprises, or further comprises, a surface carbohydrate binding protein (for example, a solute binding proteins). The surface carbohydrate binding proteins can allow a more effective binding and interaction with the gut mucosa by binding to cell surface glycosylation of the gut mucosa and or mucous layers. This binding of surface carbohydrate can then exclude the binding of pathogenic bacteria.

In alternative embodiments, a composition, formulation or pharmaceutical formulation as provided herein is dried (for example, by spray-drying or freeze-drying), and formulated into a unit dose medicament, such as a packet, sachet, orally disintegrating tablet, food stuff, capsule, lozenge, effervescent tablet, etc. The unit dose medication can be formed from a variety of materials including without limitation plastic or paper. In some embodiments, the unit dose medicament comprises a moisture barrier and/or oxygen barrier layer.

In various embodiments, a composition, formulation or pharmaceutical formulation as provided herein is in a form for anal delivery, such as a suppository or in an enema. In alternative embodiments, the composition is packaged in sachets made using a moisture and/or oxygen impermeable polymer. These sachets can be backfilled with a protective gas, such as nitrogen or argon.

In alternative embodiments, a composition, formulation or pharmaceutical formulation as provided herein is provided or formulated in a dry powder formulation, a solution, a suspension, or in a tablet or capsule format with or without an enteric coating. The dry powder can be freeze-dried or spray dried. The freeze-dried compositions are preferably frozen in the presence of a suitable cryoprotectant. The cryoprotectant can be, for example, glucose, lactose, raffinose, sucrose, trehalose, adonitol, glycerol, mannitol, methanol, polyethylene glycol, propylene glycol, ribitol, alginate, bovine serum albumin, carnitine, citrate, cysteine, dextran, dimethyl sulfoxide, sodium glutamate, glycine betaine, glycogen, hypotaurine, peptone, polyvinyl pyrrolidone, or taurine. The enteric coatings include, but are not limited to, fatty acids, waxes, shellac, plastics, plant fibers, methyl acrylate-methacrylic acid copolymers, cellulose acetate succinate, hydroxy propyl methyl cellulose phthalate, hydroxy propyl methyl cellulose acetate succinate, polyvinyl acetate phthalate (PVAP), methyl meth acrylate-methacrylic acid copolymers, cellulose acetate trimellitate, sodium alginate, and Zein.

In alternative embodiments, a microbe used in a composition, formulation or pharmaceutical formulation as provided herein, or method as provided herein, is mixed with a cryopreservative, for example, a trehalose or glycerol, optionally under anaerobic conditions, optionally frozen by processes such as, but not limited to, rapid freezing (chilling with liquid nitrogen), or by a controlled temperature reduction in a cryopreservation freezing system. Once frozen, the microbes can be dehydrated under vacuum using a process that best maintains the integrity of the microbe cells. The microbe concentration in the dry powder can be from 1 million to 500 billion cfu/g. In some embodiments, the dry powder can be from 1 billion to 100 billion cfu/g, and in a most preferred embodiment the dry powder can be from 1 billion to 50 billion cfu/g.

In alternative embodiments, the powdered microbe is resuspended in an edible oil, and exemplary edible oils include, but are not limited to: triglyceride oils (for example, vegetable oil, olive oil, and medium chain triglycerides), diglyceride oils, monoglyceride oil, and/or silicone oils.

In alternative embodiments, a prebiotic or synbiotic composition, nutrient or drug as provided herein can be dissolved in a polar liquid such as, but not limited to, water, physiological saline, mammalian milk (such as human breast milk), or an infant formula, and provided in a liquid form while the microbes are provided separately as a powder or suspension in a carrier liquid which may include a solution comprising the prebiotics or synbiotics as provided herein.

In alternative embodiments, the microbes and oligosaccharide compositions as used in a composition, formulation or pharmaceutical formulation as provided herein, or method as provided herein, is in a combined form or formulation or is provided separately. In some embodiments, the microbe is combined with an oligosaccharide in a single dose packet containing from about 1 to about 100 billion cfu of microbe and from about 0.1 to about 20 g of a prebiotic or synbiotic.

In alternative embodiments of a composition, formulation or pharmaceutical formulation as provided herein, or method as provided herein:
the composition, formulation or pharmaceutical formulation is formulated or manufactured as or in: a nano-suspension delivery system; an encochleated formulation; or, as a multilayer crystalline, spiral structure with no internal aqueous space;
the composition, formulation or pharmaceutical formulation is formulated or manufactured as a delayed or gradual enteric release composition or formulation, and optionally the formulation comprises a gastro-resistant coating designed to dissolve at a pH of 7 in the terminal ileum, optionally an active ingredient is coated with an acrylic based resin or equivalent, optionally a poly(meth)acrylate, optionally a methacrylic acid copolymer B, NF, optionally EUDRAGIT S™ (Evonik Industries AG, Essen, Germany), which dissolves at pH 7 or greater, optionally comprises a multimatrix (MMX) formulation, and optionally manufactured as enteric coated to bypass the acid of the stomach and bile of the duodenum;
the composition, formulation or pharmaceutical formulation is formulated or manufactured as a delayed release, an extended release, or a gradual enteric release composition or formulation, optionally formulated using CAPSUGEL™ (Lonza)
the plurality of non-pathogenic colony forming live bacteria used in a composition, formulation or pharmaceutical formulation as provided herein, or a method as provided herein, are substantially dormant colony forming live bacteria, or the plurality of non-pathogenic colony forming live bacteria or the plurality of non-pathogenic germinable bacterial spores are lyophilized, wherein optionally the dormant colony forming live bacteria comprise live vegetative bacterial cells that have been rendered dormant by lyophilization, spray drying, or freeze drying;
the composition, formulation or pharmaceutical formulation comprises at least about 1×10⁴ colony forming units (CFUs), or between about 1×10¹ and 1×10¹³ CFUs, 1×10² and 1×10¹⁰ CFUs, 1×10² and 1×10⁸ CFUs, 1×10³ and 1×10⁷ CFUs, or 1×10⁴ and 1×10⁶ CFUs, of non-pathogenic live bacteria and/or non-pathogenic germinable bacterial spores;
the composition, formulation or pharmaceutical formulation comprises at least one (optionally, as in a synbiotic, or combination of one species and a probiotic, optionally a synbiotic combination as set forth in Table 8 or Table 32) (or any one, several, or all of) non-pathogenic bacteria or spore of the family or genus (or class): Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), Acetatifactor (TaxID: 1427378), Adlercreutzia (TaxID: 447020), Agathobacter (TaxID: 1766253), Anaerotruncus (TaxID: 244127), Bariatricus (TaxID: 1924081), Butyrivibrio (TaxID: 830), Christensenellaceae (TaxID: 990719), Clostridiales (TaxID: 186802), Dialister (TaxID: 39948), Drancourtella (TaxID: 1903506), Eggerthella (TaxID: 84111), Eisenbergiella (TaxID: 1432051), Enterocloster (TaxID: 2719313), Enterococcus (TaxID: 1350), Intestinibacter (TaxID: 1505657), Lachnospira (TaxID: 28050), Lachnospiraceae (TaxID: 186803), Mediterraneibacter (TaxID: 2316020), Negativibacillus (TaxID: 1980693), Oscillibacter (TaxID: 459786), Phocaeicola (TaxID: 909656), Pseudobutyrivibrio (TaxID: 46205), Pseudoflavonifractor (TaxID: 1017280), Ruminococcaceae (TaxID: 541000), Sellimonas (TaxID: 1769710), Solobacterium (TaxID: 123375), Terrisporobacter (TaxID: 1505652), Tidjanibacter (TaxID: 1929083), Veillonella (TaxID: 29465), Lacticaseibacillus (TaxID: 2759736), Limosilactobacillus (TaxID: 2742598), or a combination or mix (or consortium) thereof.
the composition, formulation or pharmaceutical formulation comprises at least one (or any one, several, or all of) non-pathogenic bacteria or spore form thereof as set forth in Table 1 or Table 4, or live biotherapeutic compositions (also called probiotic) or combinations or mix (or consortium) of bacteria as set forth in Table 2 or Table 30;
the composition, formulation or pharmaceutical formulation comprises combination of at least one non-pathogenic bacteria and/or spores thereof (or spore derived from) as set forth in Table 1 or Table 4, or live biotherapeutic compositions (also called probiotic) or combinations or mix (or consortium) of bacteria as set forth in Table 2 or Table 30; and/or
the composition, formulation or pharmaceutical formulation comprises water, sterile water, saline, sterile saline, a pharmaceutically acceptable preservative, a carrier, a buffer, a diluent, an adjuvant or a combination thereof;

In alternative embodiments, the methods further comprise administering a prebiotic or synbiotic (for example, a mixture of prebiotic and probiotic as set forth in Table 8 or Table 32), nutrient, infant formula or a drug such as an antibiotic or anti-cancer agent to the subject. In alternative embodiments, compositions, formulations and pharmaceutical compounds as provided herein comprise, or are mixed with, or are formulated with, prebiotics or synbiotics (for example, a mixture of prebiotic and probiotic as set forth in Table 8 or Table 32), nutrients or a drug such as an antibiotic.

In alternative embodiments, the prebiotic or synbiotic (for example, a mixture of prebiotic and probiotic as set forth in Table 8 or Table 32) augments the growth of the anti-inflammatory bacterial population present in the probiotic composition. In alternative embodiments, the prebiotic or synbiotic augments the growth of a healthy gut microbiome, or promotes restoration of a healthy gut microbiome.

In alternative embodiments, the prebiotic or synbiotic (for example, a mixture of prebiotic and probiotic as set forth in Table 8 or Table 32) comprises a monomer or polymer selected from the group consisting of arabinoxylan, xylose, soluble fiber dextran, soluble corn fiber, polydextrose, lactose, N-acetyl-lactosamine, glucose, and combinations thereof. In one embodiment of the foregoing aspect, the prebiotic or synbiotic comprises a monomer or polymer selected from the group consisting of galactose, glucose, lactose, fructose, rhamnose, mannose, uronic acids, fucose, sialic acid, N-acetylglucosamine, 2′-fucosyllactose, lacto-N-tetraose, 3′-fucosyllactose, 3′ sialyllactose, 6′-sialyllactose, lacto-N-neotetraose, 2′,3-di-fucosyllactose, and combinations thereof. In one embodiment of the foregoing aspect, the prebiotic or synbiotic comprises a monosaccharide selected from the group consisting of arabinose, fructose, fucose, lactose, galactose, glucose, mannose, D-xylose, xylitol, ribose, and combinations thereof. In one embodiment of the foregoing aspect, the prebiotic or synbiotic comprises a disaccharide selected from the group consisting of xylobiose, sucrose, maltose, lactose, lactulose, trehalose, cellobiose, and combinations thereof. In one embodiment of the foregoing aspect, the prebiotic or synbiotic comprises a polysaccharide, wherein the polysaccharide is xylooligosaccharide. In one embodiment of the foregoing aspect, the prebiotic or synbiotic comprises a sugar selected from the group consisting of arabinose, fructose, fucose, lactose, galactose, glucose, mannose, D-xylose, xylitol, ribose, xylobiose, sucrose, maltose, lactose, lactulose, trehalose, cellobiose, xylooligosaccharide, and combinations thereof.

In alternative embodiments, compositions, formulations, or pharmaceutical compositions as provided herein or as used in methods as provided herein:
are administered orally, topically, by aerosol, sublingually, or rectally or are formulated for oral, topical, aerosol, sublingual or rectal administration, or are formulated and/or administered as a freeze-dried composition, a liposome, a liquid, a food, a gel, a supplement, a gummy, a candy, an ice, a lozenge, a tablet, pill or capsule, or a suppository or as an enema formulation, or the formulation is administered as an or is in a form for intra-rectal or intra-colonic administration;
are formulated or mixed in an infant's or child's food, drink, nutritional supplement or beverage, for example, compositions, formulations, or pharmaceutical compositions as provided herein are formulated or mixed into milk (for example, human milk, cow's milk or soy protein, and optionally fortified with vitamins, minerals, and other nutrients), infant formula, soy-based formulas, amino acid-based formulas, hydrolyzed infant formula (made from cow's milk or soy protein that has been broken down into smaller proteins that are easier for infants to digest), supplemental (harvested) human mother's milk, and the like, these can be supplemented with DHA or docosahexaenoic acid, or any omega-3 fatty acid, or iron drops;
are administered to the individual in need thereof in one, two, three, or four or more doses, and wherein the one, two, three, four or five or more doses are administered on a daily basis (optionally once a day, bid or tid or more), every other day, every third day, or about once a week, and optionally the two, three, or four or more doses are administered at least a week apart (or dosages are separated by about a week);
the compositions, formulations, or pharmaceutical compositions as provided herein or as used in methods as provided herein, further comprise a drug, for example, an antibiotic, or the method further comprises administration of the drug (for example, an antibiotic), and optionally at least one dose of the drug (for example, an antibiotic) is administered before a first administration of the compositions, formulations, or pharmaceutical compositions as provided herein, optionally at least one dose of the antibiotic is administered one day or two days, or more, before a first administration of the compositions, formulations, or pharmaceutical compositions as provided herein;
the compositions, formulations, or pharmaceutical compositions as provided herein or as used in methods as provided herein, further comprise a drug, for example, an inhibitor of the inhibitory immune checkpoint molecule, which can comprise a protein or polypeptide that binds to an inhibitory immune checkpoint protein, and optionally an inhibitor of the inhibitory immune checkpoint protein is an antibody or an antigen binding fragment thereof that specifically binds to the inhibitory immune checkpoint protein;
and optionally the inhibitor of the inhibitory immune checkpoint molecule targets a compound or protein comprising: a CTLA4 or CTLA-4 (cytotoxic T-lymphocyte-associated protein 4, also known as CD152, or cluster of differentiation 152); Programmed cell Death protein 1, also known as PD-1 or CD279; Programmed Death-Ligand 1 (PD-L1), also known as cluster of differentiation 274 (CD274) or B7 homolog 1 (B7-H1)); PD-L2; A2AR (adenosine A2A receptor, also known as ADORA2A); B7-H3; B7-H4; BTLA (B- and T-lymphocyte attenuator protein); KIR (Killer-cell Immunoglobulin-like Receptor); IDO (Indoleamine-pyrrole 2,3-dioxygenase); LAG3 (Lymphocyte-Activation Gene 3 protein); TIM-3; VISTA (V-domain Ig suppressor of T cell activation protein); or any combination thereof;
and optionally the inhibitor of an inhibitory immune checkpoint molecule comprises: ipilimumab or YERVOY®; pembrolizumab or KEYTRUDA®; nivolumab or OPDIVO®; atezolizumab or TECENTRIQ®; avelumab or BAVENCIO®; durvalumab or IMFINZI®; AMP-224 (MedImmune), AMP-514 (an anti-programmed cell death 1 (PD-1) monoclonal antibody (mAb) (MedImmune)), PDR001 (a humanized mAb that targets PD-1), STI-A1110 or STI-A1010 (Sorrento Therapeutics), BMS-936559 (Bristol-Myers Squibb), BMS-986016 (Bristol-Myers Squibb), TSR-042 (Tesaro), JNJ-61610588 (Janssen Research & Development), MSB-0020718C, AUR-012, enoblituzumab (also known as MGA271) (MacroGenics, Inc.), MBG453, LAG525 (Novartis), BMS-986015 (Bristol-Myers Squibb), cemiplimab (or LIBTAYO®) (Regeneron), or any combination thereof;
and optionally the stimulatory immune checkpoint molecule comprises a member of the tumor necrosis factor (TNF) receptor superfamily, optionally CD27, CD40, OX40, GITR (a glucocorticoid-Induced TNFR family Related gene protein) or CD137, or comprises a member of the B7-CD28 superfamily, optionally CD28 or Inducible T-cell co-stimulator (ICOS).
and optionally the drug, nutrient or prebiotic or synbiotic is administered by: aerosol, spray, intravenous (IV) injection, intramuscular (IM) injection, intratumoral injection or subcutaneous injection; or, is administered orally or by suppository; or the formulation further comprises at least one immune checkpoint inhibitor;
and optionally compositions, formulations or pharmaceutical compositions as provided herein are administered to treat or ameliorate a condition or a disease such as a cancer for failure to thrive, or are administered to augment the drug or therapy administered to an individual in need thereof for treatment of the condition or the disease,
and optionally the cancer is melanoma, advanced melanoma, cutaneous or intraocular melanoma, primary neuroendocrine carcinoma of the skin, breast cancer, a cancer of the head and neck, uterine cancer, rectal and colorectal cancer, a cancer of the head and neck, cancer of the small intestine, a colon cancer, a cancer of the anal region, a stomach cancer, lung cancer, brain cancer, non-small-cell lung cancer, ovarian cancer, angiosarcoma, bone cancer, osteosarcoma, prostate cancer; cancer of the bladder; cancer of the kidney or ureter or renal cell carcinoma, or carcinoma of the renal pelvis; a neoplasm of the central nervous system (CNS) or renal cell carcinoma;
and optionally the disease or condition is Necrotizing enterocolitis (NEC),
and optionally the disease or condition is irritable bowel disease, irritable bowel syndrome (IBD), celiac disease, gastroesophageal reflux disease (GERD) or Crohn's disease,
and optionally the disease or condition is an autoimmune disease, wherein optionally the autoimmune disorder is an allergy such as a food, pollen or drug allergy, asthma, diabetes, Crohn's Disease, Diabetes Type 1, Multiple Sclerosis, Myasthenia Gravis, Rheumatoid Arthritis, Lupus, Scleroderma, and/or Psoriasis,
and optionally the disease or condition is obesity, metabolic syndrome, type I or type II diabetes, or pre-diabetic syndrome,
and optionally the disease or condition is arthritis, inflammatory arthritis or gout,
and optionally the disease or condition is a dermatological disorder, for example, psoriasis, urticaria or angioedema;
and optionally the disease or condition is a neurological disease, for example, anxiety disorder, autism, stress, bipolar syndrome, depression, psychosis, essential tremor, Tourette's syndrome, Huntington's disease, multiple sclerosis or other demyelinating disease, organic psychosis, obsessive compulsive disorder, Alzheimer's disease or Parkinson's disease,
and optionally the method comprises, or further comprises, administering, or having administered, or delivering, a genetically (or recombinantly) engineered cell, wherein optionally the genetically engineered cell is: a microbe or spore derived from a microbe as used in a method of any of the preceding claims, or a method as provided herein; or, a non-pathogenic bacteria or spore form thereof as set forth in Table 1 or Table 4, or live biotherapeutic compositions (also called probiotic) or combinations or mix (or consortium) of bacteria as set forth in Table 2 or Table 30,
and optionally the disease or condition is an infection or a symptom or long-term sequelae of an infection (for example, long COVID), and optionally the infection is a viral, protozoan, fungal or a bacterial infection,
and optionally the microbe is genetically engineered to express or secrete a heterologous or overexpress an endogenous immunomodulatory molecule, and optionally the immunomodulatory molecule is an immunomodulatory protein or peptide, and optionally the immunomodulatory molecule is an immunostimulatory molecule,
and optionally the microbe is genetically engineered to overexpress a pathway for production of at least one short chain fatty acid (SCFA), and optionally the SCFA comprises butyrate or butyric acid, propionate or acetate,
and optionally, the microbe is genetically engineered to express a catabolic path that provides an environment niche such as human milk oligosaccharide consumption,
and optionally, the microbe is genetically engineered to express a catabolic path that shifts SCFA balance, either through consumption of production of an SCFA
and optionally, the microbe is genetically engineered to consume prebiotics or synbiotics and produce postbiotics, such as the consumption of ellagic acid to produce urolithin A or the consumption of tryptophan to produce indole-3-lactate,
and optionally the microbe is genetically engineered by inserting a heterologous nucleic acid into the microbe, and optionally the heterologous nucleic acid encodes an exogenous membrane protein,
and optionally the immunostimulatory molecule, protein or peptide comprises a non-specific immunostimulatory protein, and optionally the non-specific immunostimulatory protein comprises a cytokine, and optionally the cytokine comprises an interferon (optionally an IFN-α2a, IFN-α2b), and interleukin (optionally IL-2, IL-4, IL-7, IL-12), an interferon (IFN), a TNF-α, a granulocyte colony-stimulating factor (G-CSF, also known as filgrastim, lenograstim or Neupogen®), a granulocyte monocyte colony-stimulating factor (GM-CSF, also known as molgramostim, sargramostim, LEUKOMAX®, MIELOGEN® or LEUKINE®), or any combination thereof,
and optionally the immunostimulatory molecule, protein or peptide comprises a specific immunostimulatory protein or peptide, and optionally the specific immunostimulatory protein or peptide comprises an immunogen that can generate a specific humoral or cellular immune response or an immune response to a cancer antigen,
and optionally the genetically engineered cell is a lymphocyte, and optionally the genetically engineered cell expresses a chimeric antigen receptor (CAR), and optionally the lymphocyte is a B cell or a T cell (CAR-T cell), and optionally the lymphocyte is a tumor infiltrating lymphocyte (TIL),
and optionally the microbe is genetically engineered to substantially decrease, reduce or eliminate the microbe's toxicity,
and optionally the microbe is genetically engineered to comprise a kill switch so the microbe can be rendered non-vital after administration of an appropriate trigger or signal,
and optionally the microbe is genetically engineered to secrete anti-inflammatory compositions or have an anti-inflammatory effect,
and optionally the genetically engineered cell is administered or delivered before administration of, simultaneously with, and/or after administration or delivery of the formulation.

In alternative embodiments, provided are formulations or pharmaceutical compositions comprising:
(a) a combination or mix (or consortium) of microbes as set forth in Table 1 or Table 4, or live biotherapeutic compositions (also called probiotic) or combinations of bacteria as set forth in Table 2 or Table 30;
(b) a combination or mix (or consortium) of microbes as used in a method as provided herein or as provided herein;/or and
(c) one (for example, as in a synbiotic, or combination of one species and a probiotic, such as a synbiotic combination as set forth in Table 8 or Table 32), or at least two different, species or genera (or types) of non-pathogenic bacteria, wherein each of the non-pathogenic bacteria comprise (or are in the form of) a plurality of non-pathogenic colony forming live bacteria, a plurality of non-pathogenic germinable non-pathogenic bacterial spores, or a combination or mix (or consortium) thereof, and the formulation comprises at least one (or any one, several, or all of) non-pathogenic bacteria or spore of the family or genus (or class) as set forth in Table 1 or Table 4, or live biotherapeutic compositions (also called probiotic) or combinations, mixes or consortia of bacteria as set forth in Table 2 or Table 30; or:

Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), Acetatifactor (TaxID: 1427378), Adlercreutzia (TaxID: 447020), Agathobacter (TaxID: 1766253), Anaerotruncus (TaxID: 244127), Bariatricus (TaxID: 1924081), Butyrivibrio (TaxID: 830), Christensenellaceae (TaxID: 990719), Clostridiales (TaxID: 186802), Dialister (TaxID: 39948), Drancourtella (TaxID: 1903506), Eggerthella (TaxID: 84111), Eisenbergiella (TaxID: 1432051), Enterocloster (TaxID: 2719313), Enterococcus (TaxID: 1350), Intestinibacter (TaxID: 1505657), Lachnospira (TaxID: 28050), Lachnospiraceae (TaxID: 186803), Mediterraneibacter (TaxID: 2316020), Negativibacillus (TaxID: 1980693), Oscillibacter (TaxID: 459786), Phocaeicola (TaxID: 909656), Pseudobutyrivibrio (TaxID: 46205), Pseudoflavonifractor (TaxID: 1017280), Ruminococcaceae (TaxID: 541000), Sellimonas (TaxID: 1769710), Solobacterium (TaxID: 123375), Terrisporobacter (TaxID: 1505652), Tidjanibacter (TaxID: 1929083), Veillonella (TaxID: 29465), Lacticaseibacillus (TaxID: 2759736), Limosilactobacillus (TaxID: 2742598), or a combination thereof.

In alternative embodiments, of compositions, formulations or pharmaceutical compositions as provided herein, or methods as provided herein:
the compositions, formulations or pharmaceutical compositions comprises at least one (or any one, several, or all of) non-pathogenic bacteria or spore form thereof as set forth in Table 1 or Table 4, or live biotherapeutic compositions (also called probiotic) or combinations or mix (or consortium) of bacteria as set forth in Table 2 or Table 30, optionally also formulated or mixed with a prebiotic or synbiotic (for example, as listed in Table 3), nutrient and/or drug;
the compositions, formulations or pharmaceutical compositions comprises an inner core surrounded by an outer layer of polymeric material enveloping the inner core, wherein the non-pathogenic bacteria or the non-pathogenic germinable bacterial spores are substantially in the inner core, and optionally the polymeric material comprises a natural polymeric material;
the plurality of non-pathogenic colony forming live bacteria are substantially dormant colony forming live bacteria, or the plurality of non-pathogenic colony forming live bacteria or the plurality of non-pathogenic germinable bacterial spores are lyophilized, wherein optionally the non-pathogenic dormant colony forming live bacteria comprise live vegetative bacterial cells that have been rendered dormant by lyophilization or freeze drying;
the compositions, formulations or pharmaceutical compositions comprise at least 1×10⁴ colony forming units (CFUs), or between about 1×10³ and 1×10¹⁰ CFUs, or between about 1×10² and 1×10⁸ CFUs, 1×10³ and 1×10⁷ CFUs, or 1×10⁴ and 1×10⁶ CFUs, of live non-pathogenic bacteria and/or non-pathogenic germinable bacterial spores;
the compositions, formulations or pharmaceutical compositions comprise water, saline, a pharmaceutically acceptable preservative, a carrier, a buffer, a diluent, an adjuvant or a combination thereof;
the compositions, formulations or pharmaceutical composition are formulated for administration orally or rectally, or is formulated as a liquid, an aerosol, a spray, a powder, a food, a supplement, a nutritional aid, a medicinal food, a gel, a gel tab, a candy (for example, a lollipop), a lozenge, a tablet, pill or capsule, or a suppository;
the compositions, formulations or pharmaceutical compositions further comprise: a biofilm disrupting or dissolving agent, an antibiotic, an inhibitor of an inhibitory immune checkpoint molecule and/or a stimulatory immune checkpoint molecule (or any composition for use in checkpoint blockade immunotherapy), and
optionally the inhibitor of an inhibitory immune checkpoint molecule comprises a protein or polypeptide that binds to an inhibitory immune checkpoint protein, and optionally the inhibitor of the inhibitory immune checkpoint molecule is an antibody or an antigen binding fragment thereof that binds to an inhibitory immune checkpoint protein, as described above.

In alternative embodiments, provided are kits or products of manufacture comprising a formulation or pharmaceutical composition as provided herein, wherein optionally the product of manufacture is an implant.

In alternative embodiments, provided are uses of a formulation or pharmaceutical composition as provided herein, or a kit or product of manufacture as provided herein, for controlling, ameliorating, preventing or treating a cancer in an individual in need thereof.

In alternative embodiments, provided are uses of a composition, formulation or a pharmaceutical composition as provided herein in the manufacture of a medicament for controlling, ameliorating, preventing or treating a cancer in an individual in need thereof.

In alternative embodiments, provided are compositions, formulations or pharmaceutical compositions as provided herein, or a kit as provided herein, for use in controlling, ameliorating, preventing or treating dysbiosis in an infant that can lead to disease. Diseases in infants that have been associated with dysbiosis include but are not limited to, diabetes, obesity, allergies, asthma, autism, and eczema.

In alternative embodiments, provided are compositions, formulations or pharmaceutical compositions as provided herein, or a kit as provided herein, for use in controlling, ameliorating, preventing or treating dysbiosis in an adult that can lead to disease. Diseases in adults that have been associated with dysbiosis include but are not limited to, cancer, diabetes, obesity, allergies, asthma, gout, Alzheimer's disease, and Parkinson's disease.

In alternative embodiments, provided are compositions, formulations or pharmaceutical compositions as provided herein, or a kit as provided herein, for use in controlling, ameliorating, preventing or treating dysbiosis that can impact health outcomes for expectant mothers and their children, including their infants, wherein optionally the compositions, formulations or pharmaceutical compositions are administered to treat a failure to thrive in an infant or child, or are administered to a healthy infant or child to increase or augment health or ability to thrive.

In alternative embodiments, provided are compositions, formulations or pharmaceutical compositions as provided herein, or a kit as provided herein, for use in controlling, ameliorating, preventing or treating dysbiosis that can impact the efficacy of a pharmaceutical treatment.

In alternative embodiments, provided are compositions, formulations or pharmaceutical compositions as provided herein, or a kit as provided herein, for use in controlling, ameliorating, preventing or treating a cancer in an individual in need thereof. In alternative embodiments, the cancer is melanoma, advanced melanoma, cutaneous or intraocular melanoma, primary neuroendocrine carcinoma of the skin, breast cancer, a cancer of the head and neck, uterine cancer, rectal and colorectal cancer, a cancer of the head and neck, cancer of the small intestine, a colon cancer, a cancer of the anal region, a stomach cancer, lung cancer, brain cancer, non-small-cell lung cancer, ovarian cancer, angiosarcoma, bone cancer, osteosarcoma, prostate cancer; cancer of the bladder; cancer of the kidney or ureter or renal cell carcinoma, or carcinoma of the renal pelvis; a neoplasm of the central nervous system (CNS) or renal cell carcinoma.

The details of one or more exemplary embodiments of the invention are set forth in the accompanying drawings and the description below. Other features, objects, and advantages of the invention will be apparent from the description and drawings, and from the claims.

All publications, patents, patent applications, and GenBank and NCBI RefSeq assembly sequences and sequence references cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1 graphically illustrates sample and cluster relationships from the MY BABY BIOME™ Study. Distances were measured between every pair of 289 infant gut microbiome samples using gUniFrac. Hierarchical clustering was performed using these distances. Hierarchical clustering resulted in 3 clusters (C1, C2, and C3), each containing microbiomes of broad similarity. Principal coordinate analysis was performed to visualize how the samples and clusters related to each other.

FIG. 2 demonstrates the average abundance of 6 phyla in each of the 3 clusters for 289 infant gut samples. C1 is rich in Actinobacteriota, the phylum that contains Bifidobacterium and represents the expected infant microbiome. C2 has an enrichment in Bacteroidota (the phylum which includes Bacteroides), typical of a more mature gut-microbiome, dysbiotic for an infant. C3 is enriched in Firmicutes and Proteobacteria and is classified as a dysbiotic state.

FIG. 3 illustrates ternary plot generated by describing 287 samples infant gut samples as a 2-dimensional simplex vector by consolidating its relative abundances of Actinobacteriota, Bacteroidota, and the combination of the various Firmicutes and Proteobacteria Phyla. 2 samples were dropped that were less than 90% composition of this set of phyla. The dysbiotic C3 state is primarily in the top corner, while canonical infant microbiome would be near the bottom right amongst the C1 samples.

FIG. 4 recapitulates the PCoA plot from FIG. 1 with symbols that show the grouping of samples by birth-mode. The top right lobe where C3 was in FIG. 1 also shows an enrichment of C-Section infants, while the C2 region (bottom right) which is typical of a more mature gut-microbiome shows an enrichment in vaginally born infants, consistent with the observation that vaginal birth frequently results in vertical transmission of microbiota.

FIG. 5 recapitulates the ternary plot from FIG. 3 with symbols showing birth-method. We see that infants born by C-Section typically have exceptionally low numbers (levels) of Bacteroidota, a striking example of the lack of vertical transmission from mother to child during C-Section births.

FIG. 6 recapitulates the PCoA plot from FIG. 1 with symbols indicating the feeding mode for the infant. We see that the leftmost lobe (which was where C1 is located) has an abundance of samples from breast-fed and mixed-fed infants. This result is consistent with observations that Bifidobacterium have an increased ability to metabolize the HMOs found in breast milk.

FIG. 7 illustrates a dendrogram generated by the hierarchical clustering of 289 infant gut-microbiome samples. This was produced by measuring distances between samples with gUniFrac and performing hierarchical clustering using the Ward method. We see the C3 cluster at the top with an enrichment of C-Section born infants, followed by the C2 cluster having an enrichment of vaginally born infants, and lastly the C1 cluster.

FIG. 8 recapitulates the dendrogram from FIG. 7 but instead labels the samples with the predominant feeding mode for the infant: breast, mixed, or formula. This dendrogram was produced by measuring distances between samples with gUniFrac and performing hierarchical clustering using the Ward method.

FIG. 9 recapitulates the PCoA from FIG. 1 with samples shaded by Bifidobacterium abundance. We see the leftmost lobe (location of C1) is highest in Bifidobacterium.

FIG. 10 graphically illustrates a volcano plot showing which taxonomic groups were enriched in the C1 cluster. We measure the average fold-change and the Mann-Whitney-U p-value for each taxonomic group across the 289 infant gut microbiome samples (every species, genus, family, . . . , phylum in our data) for C1 samples vs other samples. Plot shows-log 10 (p-value) vs log 2 (fold-change). We randomized the data to find an appropriate significance threshold and taxa below that threshold were ignored (circles). We ignore taxa above the significance threshold if the group has less than 0.5% abundance in more than 10% of the samples (diamonds). Using bootstrapping we removed samples that did not have a consistent enrichment on resampling the data, this removes taxa that are dominated by outliers (non-robust “x” symbols). The remaining taxonomic groups are considered enriched (squares) or depleted (plusses).

FIG. 11 graphically illustrates strip plots of relative abundances of select taxonomic groups enriched in C1 as measured across 289 infant gut microbiome samples. Taxonomic group selection followed the procedure for FIG. 10; from the enriched taxonomic groups we selected 10 representative groups. We plot all samples and separate them by cluster. 9 of the 10 taxa are species level and the 10th is the genus Collinsella. Of the 9 species level enriched taxa shown 8 are Bifidobacterium.

FIG. 12 graphically illustrates strip plots of select species depleted in C2 compared to C1. Taxonomic group selection followed the procedure for FIG. 10; from the enriched taxonomic groups we selected 8 representative species.

FIG. 13 graphically illustrates strip plots of select species depleted in C3 compared to C1. Taxonomic group selection followed the procedure for FIG. 10; from the enriched taxonomic groups we selected 11 representative species.

FIG. 14 graphically illustrates strip plots of select taxa enriched in C3 compared to C1. Taxonomic group selection followed the procedure described in FIG. 10 and representative taxa are shown here.

FIG. 15 graphically illustrates the distribution of combined B. infantis, B. longum, B bifidum and B. breve distributed across 289 infant gut microbiomes. We see the largest fraction of the population is in the 0-10% abundance category for total consortia abundance.

FIG. 16 graphically illustrates the data from FIG. 15 with infants separated into two cohorts according to birth mode. We see that Bifidobacterium consortia abundance is lower in C-section born infants.

FIG. 17 graphically illustrates the data from FIG. 15 with infants separated into three cohorts according to feeding mode. We see a decreased probability of high consortia abundance for formula-fed infants and a bimodal distribution of consortia abundance in breast-fed babies, suggesting higher levels of the Bifidobacterium consortia if they are present.

FIG. 18 graphically illustrates a clustergram showing a representative subset of 73 infant gut-microbiome samples compared to the bacterial species found at 5% or above in those samples. The species are ordered by their taxonomic organization consistent with the GTDB release 207 newick tree.

FIG. 19 graphically illustrates a Heatmap showing gene ortholog membership for HMO metabolism genes across many representative species found in infant gut-microbiomes and the novel strain PB-STR-093. The representative species genomes were downloaded from GTDB release 207. The genes are grouped into H1-H5 and Urease clusters. Strain PB-STR-093 (a B. infantis subspecies) is shown below GTDB r207 B. infantis.

FIG. 20 graphically demonstrates that feeding mode is a significant driver of metabolism. Differentially expressed metabolites were plotted (ANOVA statistics, FDR p-value less than (<) 0.05). Metabolite abundances are compared between infants who are breast fed (squares), breast and formula fed (circles) and formula fed (triangles) using the median centered log ratio (CLR) value.

FIG. 21 graphically illustrates a network analysis of B. infantis, B. breve, B. longum, B. bifidum, immune markers, and metabolites, revealing the significant interactions in the infant gut with anti-inflammatory markers. Each node (circle) represents a feature. The node is shaded based on the multi omics dataset it came from (outlined circles for bacteria, shaded circles with no outline for metabolites, shaded with outline for immune markers). Lines connecting nodes indicate both statistical significance and strength of association (shorter=larger absolute correlation coefficient) solid lines represent positive associations, while dashed lines indicate negative associations.

FIG. 22 graphically illustrates a network analysis of all Bifidobacterium species, immune markers and metabolites, revealing that our core Bifidobacterium consortia (B. infantis, B. bifidum, B. breve, B. longum) clusters tightly together and with other Bifidobacterium in the infant gut. Each node (circle) represents a feature. The node is shaded based on the multi omics dataset it came from (outlined circles for bacteria, shaded circles with no outline for metabolites, shaded with outline for immune markers). Lines connecting nodes indicate both statistical significance and strength of association (shorter=larger absolute correlation coefficient) solid lines represent positive associations, while dashed lines indicate negative associations.

FIG. 23 graphically illustrates a network analysis of all microbiome, immune markers and metabolites that reveals Proteobacteria are significantly positively associated with proinflammatory chemokine MCP-1. For this analysis, non-Bifidobacterium taxa had all reads summed at the phylum level. Here, we specifically focus on the network module containing the Proteobacteria phylum. Each node (circle) represents a feature. Lines connecting nodes indicate both statistical significance and strength of association (shorter for larger absolute correlation coefficient) solid lines represent positive associations, while dashed lines indicate negative associations.

FIG. 24 graphically illustrates a pangenomic comparison of B. infantis strains. 10 NCBI B. infantis reference strains and 1 novel isolate are shown with coincidental genes highlighted. The strains are observed to group into 2 distinct clades (C1 and C2), with C1 having a high degree of similarity within the clade. The NCBI GCA accession numbers for the strains pictured are C2-L5:GCA_001281305, C2-L4:GCA_017299595, C2-L3:GCA_017378625, C2-L2:GCA_015102215, C2-L1:GCA_018140675, C1-L5:GCA_000020425, C1-L4:GCA_902381625, C1-L3:GCA_900637215, C1-L2:GCA_000269965, C1-L1:GCA_902167885.

FIG. 25 demonstrates through Krona charts the outgrowth of a C1 gut environment in the context of human milk oligosaccharides versus formula. The gut environment maintains a C1 community structure dominated by Bifidobacterium when grown with human milk oligosaccharides, but when grown with formula the community structure diverges, shifting to a C3 community structure dominated by Firmicutes and Proteobacteria. Each Krona chart represents the overall community composition in a simulated gut environment.

FIG. 26 demonstrates through Krona charts that Bifidobacterium infantis introduction shifts the community structure in a simulated gut environment. Comparing the first two Krona charts the introduction of Bifidobacterium infantis drastically shifts the simulated gut environment from a C3 community structure to a C1 community structure. By comparing the second- and third-Krona charts, it can be seen that introduction of human milk oligosaccharide LNT further boosts the abundance of Bifidobacterium infantis in the sample.

FIG. 27 graphically demonstrates the ability of Bifidobacterium infantis to reduce the presence of pathogens or other harmful bacteria. Upon introduction of Bifidobacterium infantis into a simulated gut environment, we see a reduction in harmful or pathogenic bacteria. Shown here are levels of three different bacteria in simulated gut environments. Groups of 4 samples show levels of Escherichia coli, Streptococcus vestibularis, and Bifidobacterium infantis with indicated carbon sources and introduction of B. infantis or control (2 replicates of each). Although Bifidobacterium infantis can reduce levels of these bacteria by itself, importantly, introduction of human milk oligosaccharides such as LNT further reduces the presence of these bacteria, demonstrating the ability of Bifidobacterium infantis to suppress pathogens and other unwanted bacteria in a prebiotic dependent manner.

FIG. 28 graphically demonstrates cytokine expression with and without B. infantis. Cytokine induction was evaluated using supernatants from simulated gut environments compared to background media. When a simulated gut environment was generated with additional Bifidobacterium infantis (+ spike), a significant reduction in the induction of pro-inflammatory cytokines was observed demonstrating the anti-inflammatory nature of the microbe in the simulated gut environment.

FIG. 29 graphically illustrates the outgrowths of C3 fecal samples with and without probiotic through ternary plots. Probiotic inoculation and outgrowths of Bifidobacterium were performed to investigate restoring in vitro simulated infant gut microbiomes. Probiotic inoculation results in shifts towards higher Actinobacteriota and a more typical infant gut microbiome.

FIG. 30 graphically demonstrates the relative abundance of Bifidobacterium in in vitro outgrowths of probiotic simulated C3 infant gut microbiomes through box and whisker plots. Combo_15 is a control with no Bifidobacterium species in the inoculation while the other combinations have approximately equivalent colony forming units (CFU) of Bifidobacterium. Combo_14 had the highest outgrowth of Bifidobacterium.

FIG. 31 demonstrates through a strip plot the relative abundances of Bifidobacterium in an in vitro simulation of C3 infant gut microbiomes after stimulation with probiotic. Three different C3 samples are shown.

FIG. 32 demonstrates a pangenome analysis of B. infantis. Genomes of Persephone biosciences B. infantis strains were analyzed in combination with published B. infantis genomes to determine differentiating characteristics of strains.

FIG. 33 demonstrates a pangenome analysis of B. longum. Genomes of Persephone biosciences B. longum strains were analyzed in combination with published B. longum genomes to determine differentiating characteristics of strains.

FIG. 34 demonstrates a pangenome analysis of B. breve. Genomes of Persephone biosciences B. breve strains were analyzed in combination with published B. breve genomes to determine differentiating characteristics of strains.

FIG. 35 demonstrates a pangenome analysis of B. bifidum. Genomes of Persephone biosciences B. bifidum strains were analyzed in combination with published B. bifidum genomes to determine differentiating characteristics of strains.

FIG. 36 graphically shows differential metabolite abundance in different Bifidobacterium combinations through center log ratio (CLR) analysis. Boxplots display the distribution of CLR values for three key metabolites: Indole-3-Lactate, 4-Hydroxyphenyllactate, and Arginine, across various combinations of Bifidobacterium (‘Combo_1,’ ‘Combo_2,’ ‘Combo_4,’ etc.) introduced into simulated in vitro gut environments. Notably, Combo_15 serves as our control and does not include additional Bifidobacterium. The y-axis represents the CLR values, offering insights into the relative abundance of each metabolite, while the x-axis denotes the specific Bifidobacterium combinations. The figure demonstrates variations in metabolite abundance across different Bifidobacterium compositions, facilitating a deeper understanding of their metabolic profiles.

FIG. 37 graphically demonstrates fold change vs-log 10 (p) (Mann-Whitney U) for metadata variables in the DIABIMMUNE study. This is for fecal samples taken from individuals between 110-days and 1 year old and compares mean Bifidobacterium abundance between those with the metadata flag and those without. High significance is seen for reduced Bifidobacterium abundance in infants fed formula and those that had milk allergy or birch allergy by the time they were 3 years old.

FIG. 38 shows a scatter plot demonstrating the inverse relationship between Bifidobacterium abundance and total IGE (Spearman r=−0.185, p-value=0.013).

FIG. 39 shows a ternary plot describing “3 country cohort” data from the DIABIMMUNE study. We see low Actinobacteriota abundance in industrialized Finland compared to their rural Russian neighbors.

FIG. 40 graphically demonstrates follow up 6 month medical history surveys from MY BABY BIOME™ revealing 11 individuals with adverse skin conditions of either eczema or dermatitis. These events were not as prevalent in the high Bifidobacterium region of the PCoA region (upper left).

FIG. 41 demonstrates differences in abundance for select Bifidobacterium and combinations of Bifidobacterium for the samples from infants that developed either eczema or dermatitis vs those that didn't by the time of the 6-month survey. Statistically significant trends (Mann-Whitney U) are seen for B. bifidum.

FIG. 42 graphically illustrates a network analysis of all Bifidobacterium species, immune markers and metabolites, revealing that our core Bifidobacterium consortia (B. infantis, B. bifidum, B. breve, B. longum) clusters tightly together and with other Bifidobacterium in the infant gut. Each node (circle) represents a feature. The node is shaded based on the multi omics dataset it came from (outlined circles for bacteria, shaded circles with no outline for metabolites, shaded with outline for immune markers). Lines connecting nodes indicate both statistical significance and strength of association (shorter=larger absolute correlation coefficient) solid lines represent positive associations, while dashed lines indicate negative associations.

FIG. 43 shows flow cytometry data for four different Bifidobacterium strains produced at a seven-liter fermentation scale; cells have been binned into three categories, dead, alive, and injured.

FIG. 44 shows the association between the GUNIFRAC™ (gUniFrac) (Jun Chen et al) clusters generated from the KRAKEN2™ classified samples (C1, C2, and C3) and the DIRICHLET MULTINOMIAL MIXTURE™ models (DMM1, DMM2, and DMM3). The DMM clusters are built without knowledge of the phylogenetic tree.

FIG. 45 is a fundamental example of the difference between GUNIFRAC™ (gUniFrac) clusters and DMM clusters. C1 is characterized by high Bifidobacteria abundance and samples high in B. dentium are therefore classified as C1. With DMM clusters there is no knowledge of the phylogenetic tree used when grouping samples, only the inferred joint probability distributions. Samples high in B. dentium can be seen to now be members of DMM1, rather than DMM3 which is considered the healthy infant gut DMM cluster.

FIG. 46 shows differential abundance between DMM3 (healthy infant gut) and DMM1 and DMM2 combined. Similar analyses were done for DMM2 and DMM1. The enriched taxa for each DMM cluster are listed in Table 39.

FIG. 47 Shows the difference in the distribution of antibiotic resistance hits between the gUniFrac clusters. Samples that are classified as C1 (a healthy infant gut) tend to have lower numbers of antibiotic resistance markers.

FIG. 48 Shows the difference in the distribution of antibiotic resistance hits between the Dirichlet multinomial mixture clusters. Samples that are classified as DMM3 (a healthy infant gut) tend to have lower numbers of antibiotic resistance markers.

FIG. 49 The inverse relationship between antibiotic resistance markers and Bifidobacterium abundance.

FIG. 50 The distributions of antibiotic resistance genes found in each sample separated by feeding mode. Breast fed babies have significantly less antibiotic resistance markers.

FIG. 51 Distributions of Consortia Relative Abundance (total of B. infantis, B. bifidum, B. longum, B. breve relative abundances) separated by feeding mode and birth mode. Vaginally born, breast fed infants had the highest median consortia abundance, but formula fed C-section born infants have higher consortia abundance than formula fed vaginally born infants.

Like reference symbols in the various drawings indicate like elements.

### DETAILED DESCRIPTION

In alternative embodiments, provided are compositions, including products of manufacture and kits, and methods for using them, comprising novel combinations or mix (or consortium) of microbes, also called live biotherapeutic compositions (also called probiotic) such as non-pathogenic, live (optionally dormant) bacteria and/or bacterial spores, for example, the exemplary combinations or mix (or consortium) of microbes as listed in Table 1 or Table 4, or live biotherapeutic compositions or combinations or mix (or consortium) of bacteria as set forth in Table 2 or Table 30.

In alternative embodiments, provided are compositions, including products of manufacture and kits, and methods for using them, for:
controlling, ameliorating, lessoning or preventing the symptoms of or the mortality of a dysbiosis or an infection in an individual in need thereof,
   wherein optionally the infection is a bacterial infection or a viral infection,
   wherein optionally the dysbiosis causes or exacerbates a Failure to Thrive (FTT) of the individual, and optionally the dysbiosis is in a newborn, an infant or a mother (material dysbiosis), and optionally the newborn or infant is between 0 and 36 months old,
modulating the microbiome or changing the microbiome of an individual,
   wherein optionally the individual is a human, and optional the human is a human child or a human infant or newborn, and optionally the infant or newborn is between 0 and 36 months old,
   and optionally the microbiome of the individual is modulated to positively affects the growth, thriving or health of the individual (or increases the ability of the individual to thrive),
and optionally the microbiome of the individual is modulated to enhance the efficacy of a treatment in an individual in need thereof, wherein optionally the treatment is a drug treatment or administration, optionally the drug treatment is for cancer,
treating, ameliorating, lessoning or preventing a disease or condition caused by a dysbiosis in an individual in need thereof, wherein optionally the individual is a human, and optional the human is a human child or a human infant or newborn, and optionally the infant or newborn is between 0 and 36 months old, wherein optionally the disease or condition is a Failure to Thrive (FTT),
the method comprising:
(a) administering or having administered to an individual in need thereof, for example, a child or an infant, a formulation comprising one (for example, as in a synbiotic, or combination of one species and a probiotic, such as a synbiotic combination as set forth in Table 8 or Table 32), or at least two different species or genera (or types) of, non-pathogenic bacteria, wherein each of the non-pathogenic bacteria comprise (or are in the form of) a plurality of non-pathogenic colony forming live bacteria, a plurality of non-pathogenic germinable bacterial spores, or a combination thereof.

In alternative embodiments, the compositions, products of manufacture, kits and methods as provided herein are used as a therapy (for example, as a mono-therapy or as a co-therapy, or co-treatment) for the control, amelioration, prevention and/or treatment of a disease or condition, for example, a cancer.

In alternative embodiments, the compositions, products of manufacture, kits and/or methods as provided herein are administered to an individual receiving a drug or a therapy, for example, a cancer therapy, thereby resulting in a modification or modulation of the patient's gut microfloral population(s), thus resulting in an enhancement of the drug or other therapy, for example, lowering the dosage or amount of drug needed for effective therapy, or the frequency with which a drug must be administered to be effective.

In alternative embodiments, by modulating or modifying the individual's gut microbial population(s) using compositions, products of manufacture and methods as provided herein, the pharmacodynamics of a drug administered to the patient is altered, for example, the pharmacodynamics of the drug is enhanced, for example, the individual's ability to absorb a drug is modified (for example, accelerated or slowed, or enhanced), or the dose efficacy of a drug is increased (for example, resulting in needing a lower dose of drug for an intended effect), or the gut microbes act orthogonally on the drug target (for example, resulting in the presence of the microbe being essential for the drug to have the intended effect). For example, in alternative embodiments, by modulating or modifying the patient's gut microbial population(s) using compositions, products of manufacture and methods as provided herein the dose efficacy of a cancer drug is increased, thereby enhancing the control or treatment of that cancer.

In alternative embodiments, the amount, identity, presence, and/or ratio of gut microbiota in a subject is manipulated to facilitate a mono-therapy or one or more co-treatments; for example, in alternative embodiments, combinations or mix (or consortium) of microbes as provided herein are administered with (for example, concurrent with, or before and/or after) a chemotherapy, a radiation therapy, an immune checkpoint inhibitor, a Chimeric Antigen Receptor (CAR) T-cell therapy (CAR-T) or other immunotherapy or cancer treatment.

Described here for the first time are novel combinations or mix (or consortium) of specific microbes, for example, bacteria, for example, a Bifidobacterium or Bacillus species, optionally a Bifidobacterium infantis specie, including for example microbes (or bacteria) found in a human gut or recombinantly engineered or cultured microbes, which can be administered as a mono-therapy or as a co-therapy for, in alternative embodiments, to infants or newborns to for example increase their ability to thrive or grow or resist infection or disease, or to cancer or autoimmune patients, where in alternative embodiments the cancer patients are undergoing immune checkpoint inhibitor treatment, or are undergoing a chemotherapy, a radiation therapy, an immune checkpoint inhibitor, a Chimeric Antigen Receptor (CAR) T-cell therapy (CAR-T) or other immunotherapy or cancer treatment.

We have demonstrated a correlation between these combinations of microbes and the metabolic functions associated with them, and the efficacy of treatment in both human patients and mouse cancer models. In alternative embodiments, administering combinations of microbes as provided herein to cancerous mice improves the fraction of animals that show significant tumor size reduction as compared to mice given the same drug but not having their gut microbiome altered using compositions or methods as provided herein.

In alternative embodiments, the chemotherapy, radiation therapy, Chimeric Antigen Receptor (CAR) T-cell therapy (CAR-T) or other immunotherapy or cancer treatment, for example, the immune checkpoint inhibitors (or inhibitors of an inhibitory immune checkpoint molecule) and/or stimulatory immune checkpoint molecules (or more accurately, stimulatory immune molecules), are administered with (for example, are administered concurrently or sequentially), or formulated with, the combinations of microbes as provided herein, for example, administered or formulated with non-pathogenic bacteria and/or non-pathogenic germination-competent bacterial spores as provided herein.

The immune checkpoint inhibitors (also described as an inhibitor of an inhibitory immune checkpoint molecule) can function by interfering with regulatory pathways that naturally exist to prevent T cell proliferation. In the tumor microenvironment these inhibitory pathways are highly active, so T cells are often driven to an ineffective state. Checkpoint inhibitors bind to particular proteins in these regulatory pathways associated with inhibition of T cell activation, such as cytotoxic T lymphocyte-associated protein 4 (CTLA-4), programmed cell death protein 1 (PD-1), or programmed cell death ligand 1 (PD-L1), thereby allowing excitatory T cell response to tumor antigens. Thus, in alternative embodiments, an inhibitor of an inhibitory immune checkpoint molecule is a molecule that can directly (or specifically) bind to CTLA-4, PD-1, PD-L1, or other component of the inhibitory immune checkpoint to prevent proper binding to its natural corresponding receptor or ligand.

In alternative embodiments, a stimulatory immune checkpoint molecule, which can also be, or more accurately, is described as a stimulatory immune molecule potentiates excitation and activation of T cells, either by enhancing the action of a checkpoint inhibitor or by an independent mechanism.

In alternative embodiments, provided are therapeutic compositions, including formulations and pharmaceutical compositions, comprising non-pathogenic (optionally dormant) live microbes such as bacteria and/or germination-competent bacterial spores, which can be used for the prevention or treatment of a cancer or the side effects of a cancer therapy, for example, a drug therapy, or can be used or administered with a chemotherapy, a radiation therapy, an immune checkpoint inhibitor, a Chimeric Antigen Receptor (CAR) T-cell therapy (CAR-T) or other immunotherapy or cancer treatment.

In alternative embodiments, therapeutic compositions, formulations or pharmaceutical compositions as provided herein, or used to practice methods as provided herein, comprise colony forming (optionally dormant) live bacteria and/or germinable bacterial spores which can be used in mono- or co-therapies, for example, as an adjuvant to an antineoplastic treatment administered to a cancer patient, or administered with or as a supplement to a chemotherapy, a radiation therapy, an immune checkpoint inhibitor, a Chimeric Antigen Receptor (CAR) T-cell therapy (CAR-T) or other immunotherapy or cancer treatment.

In some embodiments, a therapeutic composition as provided herein acts or is used as a probiotic composition which can be administered with, before and/or after a chemotherapy, a radiation therapy, an immune checkpoint inhibitor, a Chimeric Antigen Receptor (CAR) T-cell therapy (CAR-T) or other immunotherapy or cancer treatment. In alternative embodiments, therapeutic compositions (for example, the formulations) as provided herein, comprise the bacteria and/or spores and an antineoplastic active agent such as an immune checkpoint inhibitor.

In alternative embodiments, therapeutic compositions, formulations or pharmaceutical compositions as provided herein, or used to practice methods as provided herein, comprise colony forming (optionally dormant) live bacteria and/or germinable bacterial spores for use as a mono-therapy or in combination with (for example, as a co-therapy) or supplementary to a drug (which can be a small molecule or a protein, for example, a therapeutic antibody) blocking an immune checkpoint for inducing immunostimulation in a cancer patient. The therapeutic composition as provided herein and the drug (for example, an antibody) can be administered separately or together, or at different time points or at the same time, or can be administered sequentially or concurrently.

In alternative embodiments, therapeutic compositions, formulations or pharmaceutical compositions as provided herein comprise colony forming (optionally dormant) live bacteria and/or germinable bacterial spores which can be used as an adjuvant to an anti-cancer or antineoplastic treatment, for example, an immune checkpoint treatment, administered to a cancer patient. In alternative embodiments, the therapeutic composition comprises the antineoplastic or immune checkpoint active agents. In alternative embodiments, the therapeutic composition, formulations or pharmaceutical compositions as provided herein are administered with or after, or both with and after, administration of the antineoplastic or immune checkpoint active agent.

In alternative embodiments, the formulation or pharmaceutical composition further comprises, or is manufactured with, an outer layer of polymeric material (for example, natural polymeric material) enveloping, or surrounding, a core that comprises the combination of microbes as provided herein.

In alternative embodiments, therapeutic compositions, formulations or pharmaceutical compositions as provided herein, or used to practice methods as provided herein, can comprise a pharmaceutically acceptable carrier, diluent, and/or adjuvant. In other embodiments a pharmaceutically acceptable preservative is present. In yet other embodiments, a pharmaceutically acceptable germinate is present. In still other embodiments the therapeutic composition contains, or further comprises, a prebiotic or synbiotic nutrient at an effective dose of about 0.005, 0.05, 0.5, 5.0 milligrams (mg) per kilogram (kg) body weight, or between about 0.005 and 10 mgm per kilogram body weight.

In alternative embodiments, therapeutic compositions, formulations or pharmaceutical compositions as provided herein, or used to practice methods as provided herein, are in the form of a tablet, gel tab or capsule, for example, a polymer capsule such as a gelatin or a hydroxypropyl methylcellulose (HPMC, or hypromellose) capsule (for example, VCAPS PLUS™ (Capsugel, Lonza)). In other embodiments, the therapeutic compositions, formulations or pharmaceutical compositions are in or are manufactured as a food or drink, for example, an ice, candy, lolly or lozenge, or any liquid, for example, in a beverage.

In alternative embodiments, therapeutic compositions, formulations or pharmaceutical compositions as provided herein, or used to practice methods as provided herein, comprise at least one bacterial type that is not detectable, of low natural abundance, or not naturally found, in a healthy or normal subject's (for example, human) gastrointestinal tract. In alternative embodiments, the gastrointestinal tract refers to the stomach, the small intestine, the large intestine and the rectum, or combinations thereof.

In alternative embodiments, provided are methods of ameliorating, preventing or treating cancer and/or at least one symptom resulting from a cancer therapy or of a condition of the gastrointestinal tract.

In alternative embodiments, by administration of a therapeutic composition, formulation or pharmaceutical composition as provided herein to a subject, or practicing a method as provided herein, the microbiome population or composition of the subject is modulated or altered.

In alternative embodiments, the term “microbiome” encompasses the communities of microbes that can live sustainably and/or transiently in and on a subject's body, for example, in the gut of a human, including bacteria, viruses and bacterial viruses, archaea, and eukaryotes. In alternative embodiments, the term “microbiome” encompasses the “genetic content” of those communities of microbes, which includes the genomic DNA, RNA (ribosomal-, messenger-, and transfer-RNA), the epigenome, plasmids, and all other types of genetic information.

In alternative embodiments, the term “subject” refers to any animal subject including humans, laboratory animals (for example, primates, rats, mice), livestock (for example, cows, sheep, goats, pigs, turkeys, and chickens), and household pets (for example, dogs, cats, and rodents). The subject may be suffering from a disease, for example, a cancer, and autoimmune disease or condition, or a failure to thrive.

In alternative embodiments, the term “type” or “types” when used in conjunction with “bacteria” or “bacterial” refers to bacteria differentiated at the genus level, the species level, the sub-species level, the strain level, or by any other taxonomic method known in the art.

In alternative embodiments, the phrase “dormant live bacteria” refers to live vegetative bacterial cells that have been rendered dormant by lyophilization or freeze drying. Such dormant live vegetative bacterial cells are capable of resuming growth and reproduction immediately upon resuscitation.

In alternative embodiments, the term “spore” also includes “endospore”, and these terms can refer to any bacterial entity which is in a dormant, non-vegetative and non-reproductive stage, including spores that are resistant to environmental stress such as desiccation, temperature variation, nutrient deprivation, radiation, and chemical disinfectants. In alternative embodiments, “spore germination” refers to the dormant spore beginning active metabolism and developing into a fully functional vegetative bacterial cell capable of reproduction and colony formation. In alternative embodiments, “germinant” is a material, composition, and/or physical-chemical process capable of inducing vegetative growth of a dormant bacterial spore in a host organism or in vitro, either directly or indirectly.

In alternative embodiments, the term “colony forming” refers to a vegetative bacterium that is capable of forming a colony of viable bacteria or a spore that is capable of germinating and forming a colony of viable bacteria.

In alternative embodiments, the term “natural polymeric material” comprises a naturally occurring polymer that is not easily digestible by human enzymes so that it passes through most of the human digestive system essentially intact until it reaches the large or small intestine.

In alternative embodiments, therapeutic compositions, formulations or pharmaceutical compositions as provided herein comprise population(s) of non-pathogenic dormant live bacteria and/or bacterial spores. The dormant live bacteria can be capable of colony formation and, in the case of spores, germination and colony formation. Thus, in alternative embodiments, compositions are useful for altering a subject's gastrointestinal biome, for example, by increasing the population of those bacterial types or microorganisms, or are capable of altering the microenvironment of the gastrointestinal biome, for example, by changing the chemical microenvironment or disrupting or degrading intestinal mucin or biofilm, thereby providing treatment of cancer, gastrointestinal conditions, and symptoms resulting from cancer therapy, ultimately increasing the health of the subject to whom they are administered.

In alternative embodiments, the terms “purify,” purified,” and “purifying” are used interchangeably to describe a population's known or unknown composition of bacterial type(s), amount of that bacterial type(s), and/or concentration of the bacterial type(s); a purified population does not have any undesired attributes or activities, or if any are present, they can be below an acceptable amount or level. In alternative embodiments, the various populations of bacterial types are purified, and the terms “purified,” “purify,” and “purifying” refer to a population of desired bacteria and/or bacterial spores that have undergone at least one process of purification; for example, a process comprising screening of individual colonies derived from fecal matter for a desired phenotype, such as their effectiveness in enhancing the pharmacodynamics of a drug (such as a cancer drug, for example, a drug inhibitory to an immune checkpoint), for example, the individual's ability to absorb a drug is modified (for example, accelerated or slowed, or enhanced), or the dose efficacy of a drug is increased (for example, resulting in needing a lower dose of drug for an intended effect), or the immune system is primed for improved drug efficacy, or a selection or enrichment of the desired bacterial types.

Enrichment can be accomplished by increasing the amount and/or concentration of the bacterial types, such as by culturing in a media that selectively favors the growth of certain types of microbes, by screening pure microbial isolates for the desired genotype, or by a removal or reduction in unwanted bacterial types.

In alternative embodiments, bacteria used to practice compositions and methods provided herein are derived from fecal material donors that are in good health, have microbial biomes associated with good health, and are typically free from antibiotic administration during the collection period and for a period of time prior to the collection period such that no antibiotic remains in the donor's system. In alternative embodiments, the donor subjects do not suffer from and have no family history of renal cancer, bladder cancer, breast cancer, prostate cancer, lymphoma, leukemia, autoimmune disease. In alternative embodiments, donor subjects are free from irritable bowel disease, irritable bowel syndrome, celiac disease, Crohn's disease, colorectal cancer, anal cancer, stomach cancer, sarcomas, any other type of cancer, or a family history of these diseases. In alternative embodiments, donor subjects do not have and have no family history of mental illness, such as anxiety disorder, depression, bipolar disorder, autism spectrum disorders, panic disorders, obsessive-compulsive disorder, attention-deficit disorders, eating disorders (for example bulimia, anorexia), mood disorder or schizophrenia. In yet other embodiments the donor subjects have no knowledge or history of food allergies or sensitivities.

In alternative embodiments, the health of fecal matter donors is screened prior to the collection of fecal matter, such as at 1, 2, 3, 4, 8, 16, 20, 24, 28, 32, 36, 40, 44, 48, or 52 weeks pre-collection. In alternative embodiments, fecal matter donors are also screened post-collection, such as at 1, 2, 3, 4, 8, 16, 20, 24, 28, 32, 36, 40-, 44-, 48-, or 52-weeks post-collection. Pre- and post-screening can be conducted daily, weekly, bi-weekly, monthly, or yearly. In alternative embodiments, individuals who do not test positive for pathogenic bacteria and/or viruses (for example HIV, hepatitis, polio, adeno-associated virus, pox, coxsackievirus, etc.) pre- and post-collection are considered verified donors.

In alternative embodiments, to purify bacteria and/or bacterial spores, fecal matter is collected from donor subjects and placed in an anaerobic chamber within a short time after elimination, such as no more than 1 minute, 5 minutes, 10 minutes, 15 minutes, 20 minutes, 25 minutes, 30 minutes, 35 minutes, 40 minutes, 45 minutes, 50 minutes, 55 minutes, or 60 minutes or more after elimination. In alternative embodiments, fecal matter samples collected from donor subjects are placed in an anaerobic chamber within between about 1 minute and 48 hours, or more, after elimination from the donor.

Bacteria from a sample of the collected fecal matter can be collected in several ways. For example, the sample can be mixed with anoxic nutrient broth, dilutions of the resulting mixture conducted, and bacteria present in the dilutions grown on solid anoxic media. Alternatively, bacteria can be isolated by streaking a sample of the collected material directly on anoxic solid media for growth of isolated colonies. In alternative embodiments, to increase the ease of isolating bacteria from fecal samples mixed with anoxic nutrient broth, the resulting mixture can be shaken, vortexed, blended, filtered, and centrifuged to break up and/or remove large non-bacterial matter.

In alternative embodiments, purification of the isolated bacteria and/or bacterial spores by any means known in the art, for example, contamination by undesirable bacterial types, host cells, and/or elements from the host microbial environment can be eliminated by reiterative streaking to single colonies on solid media until at least two replicate streaks from serial single colonies show only a single colony morphology. Purification can also be accomplished by reiterative serial dilutions to obtain a single cell, for example, by conducting multiple 10-fold serial dilutions to achieve an ultimate dilution of 10⁻², 10⁻³, 10⁻⁴, 10⁻⁵, 10⁻⁶, 10⁻⁷, 10⁻⁸, 10⁻⁹ or greater. Any methods known to those of skill in the art can also be applied.

Confirmation of the presence of only a single bacterial type can be confirmed in multiple ways such as, gram staining, PCR, DNA sequencing, enzymatic analysis, metabolic profiling/analysis, antigen analysis, and flow cytometry using appropriate distinguishing reagents.

In alternative embodiments, purified population(s) of vegetative bacteria that are incorporated into therapeutic bacterial compositions as provided herein, or used to practice methods as provided herein, are fermented in growth media. Suitable growth media include NUTRIENT BROTH™ (THERMO SCIENTIFIC™ OXOID™), ANAEROBE BASAL BROTH™ (THERMO SCIENTIFIC™ OXOID™), REINFORCED CLOSTRIDIAL MEDIUM™ (THERMO SCIENTIFIC™ OXOID™), SCHAEDLER ANAEROBIC BROTH™ (THERMO SCIENTIFIC™ OXOID™), MRS BROTH™ (MILLIPORE-SIGMA™), VEGITONE ACTINOMYCES BROTH™ (MILLIPORE-SIGMA™), VEGITONE INFUSION BROTH™ (MILLIPORE-SIGMA™), VEGITONE CASEIN SOYA BROTH™ (Millipore-Sigma™), or one of the following media available from ANAEROBE SYSTEMS™. BRAIN HEART INFUSION BROTH™ (BHI), Campylobacter-Thioglycollate Broth (CAMPY-THIO), Chopped Meat Broth (CM), Chopped Meat Carbohydrate Broth (CMC), CHOPPED MEAT GLUCOSE BROTH™ (CMG), Cycloserine Cefoxitin Mannitol Broth with Taurocholate Lysozyme Cysteine (CCMB-TAL), Oral Treponeme Enrichment Broth (OTEB), MTGE-ANAEROBIC ENRICHMENT BROTH™ (MTGE), Thioglycollate Broth with Hemin, Vit. K, without indicator, (THIO), Thioglycollate Broth with Hemin, Vit. K, without indicator, (THIO), Lactobacilli-MRS Broth (LMRS), Brucella Broth (BRU-BROTH), Peptone Yeast Extract Broth (PY), PY Glucose (PYG), PY Arabinose, PY Adonitol, PY Arginine, PY Amygdalin, PYG Bile, PY Cellobiose, PY DL-Threonine, PY Dulcitol, PY Erythritol, PY Esculin, PYG Formate/Fumarate for FA/GLCf, PY Fructose, PY Galactose, PYG Gelatin, PY Glycerol, Indole-Nitrate Broth, PY Inositol, PY Inulin, PY Lactate for FA/GLCf, PY Lactose, PY Maltose, PY Mannitol, PY Mannose, PY Melezitose, PY Melibiose, PY Pyruvic Acid, PY Raffinose, PY Rhamnose, PY Ribose, PY Salicin, PY Sorbitol, PY Starch, PY Sucrose, PY Trehalose, PY Xylan, PY Xylose, Reinforced Clostridial Broth (RCB), Yeast Casitone Fatty Acids Broth with Carbohydrates (YCFAC Broth). In alternative embodiments, growth media includes or is supplemented with reducing agents such as L-cysteine, dithiothreitol, sodium thioglycolate, and sodium sulfide. In alternative embodiments, fermentation is conducted in stirred-tank fermentation vessels, performed in either batch or fed-batch mode, with nitrogen sparging to maintain anaerobic conditions. pH is controlled by the addition of concentrated base, such as NH₄OH or NaOH. In the case of fed-batch mode, the feed is a primary carbon source for growth of the microorganisms, such as glucose. In alternative embodiments, the post-fermentation broth is collected, and/or the bacteria isolated by ultrafiltration or centrifugation and lyophilized or freeze dried prior to formulation.

In alternative embodiments, purified and isolated vegetative bacterial cells used in therapeutic bacterial compositions as provided herein, or used to practice methods as provided herein, have been made dormant; noting that bacterial spores are already in a dormancy state. Dormancy of the vegetative bacterial cells can be accomplished by, for example, incubating and maintaining the bacteria at temperatures of less than 4° C., freezing and/or lyophilization of the bacteria. Lyophilization can be accomplished according to normal bacterial freeze-drying procedures as used by those of skill in the art, such as those reported by the AMERICAN TYPE CULTURE COLLECTION™ (ATCC).

In alternative embodiments, the purified population of dormant live bacteria and/or bacterial spores has undetectable levels of pathogenic activities, such as the ability to cause infection and/or inflammation, toxicity, an autoimmune response, an undesirable metabolic response (for example diarrhea), or a neurological response.

In alternative embodiments, all of the types of dormant live bacteria or bacterial spores present in a purified population are obtained from fecal material treated as described herein or as otherwise known to those of skill in the art. In other embodiments, one or more of the types of dormant live bacteria or bacterial spores present in a purified population is generated individually in culture and combined with one or more types obtained from fecal material. In alternative embodiments, all of the types of dormant live bacteria or bacterial spores present in a purified population are generated individually in culture. In still other embodiments, one or all of the types of dormant live bacteria and/or bacterial spores present in a purified population are non-naturally occurring or engineered. In yet other embodiments, non-naturally occurring or engineered non-bacterial microorganisms are present, with or without dormant live bacteria and/or bacterial spores.

In alternative embodiments, bacterial compositions used in compositions as provided herein, or to practice methods as provided herein, comprise combinations of different bacteria, for example, comprising at least 2, 3, 4, 5, 6, 7, 8, 9, 10 or more bacterial types, or more than 20 bacterial types, or between about 2 and 30 bacterial types.

In alternative embodiments, the bacterial compositions comprise at least about 10², 10³, 10⁴, 10⁵, 10⁶, 10⁷, 10⁸, 10⁹, 10¹⁰, 10¹¹, 10¹², 10¹³, 10¹⁴, or more (or between about 10² to 10¹⁵) microbes, for example, dormant live bacteria and/or bacterial spores. In some embodiments each bacterial type is equally represented in the total number of dormant live bacteria and/or bacterial spores. In other embodiments, at least one bacterial type is represented in a higher amount than the other bacterial type(s) found in the composition.

In alternative embodiments, a population of different bacterial types used in compositions as provided herein, or to practice methods as provided herein, can increase microbe populations found in the subject's (or an individual in need thereof) gastrointestinal (GI) tract by at least about 1%, 5%, 10%, 20%, 30%, 40%, 50%, 60%, 70%, 80%, 90%, 100%, 200%, 300%, 400%, 500%, 600%, 700%, 800%, 900% or 1000%, optionally up to 10,000% or more or between about 5% and 2000%, or more or between about 1% and 10,000%, as compared to the subject's microbiome gastrointestinal population prior to treatment, wherein optionally the individual in need thereof is an infant or a newborn.

In alternative embodiments, the combination of microbes, for example, combination of bacterial cells and/or spores, used in compositions as provided herein, or to practice methods as provided herein, are mixed with pharmaceutically acceptable excipients, such as diluents, carriers, adjuvants, binders, fillers, salts, lubricants, glidants, disintegrants, coatings, coloring agents, etc. Examples of such excipients are acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium stearate, calcium disodium EDTA, copolyvidone, calcium hydrogen phosphate dihydrate, cetylpyridine chloride, cysteine HCL, crossprovidone, calcium phosphate di or tri basic, dibasic calcium phosphate, disodium hydrogen phosphate, dimethicone, erythrosine sodium, ethyl cellulose, gelatin, glyceryl monooleate, glycerin, glycine, glyceryl monostearate, glyceryl behenate, hydroxy propyl cellulose, hydroxyl propyl methyl cellulose, hypromellose, HPMC phthalate, inulin, iron oxides or ferric oxide, iron oxide yellow, iron oxide red or ferric oxide, lactose hydrous or anhydrous or monohydrate or spray dried, magnesium stearate, maltodextrin, microcrystalline cellulose, mannitol, methyl cellulose, magnesium carbonate, mineral oil, methacrylic acid copolymer, magnesium oxide, methyl paraben, providone or PVP, PEG, polysorbate 80, propylene glycol, polyethylene oxide, propylene paraben, polaxamer 407 or 188, potassium bicarbonate, potassium sorbate, potato starch, phosphoric acid, polyoxy 140 stearate, sodium starch glycolate, starch pregelatinized, sodium carmellose, sodium lauryl sulfate, starch, silicon dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dihydrate, sodium starch, sodium carboxy methyl cellulose, succinic acid, sodium propionate, titanium dioxide, talc, triacetin, and triethyl citrate.

In alternative embodiments, the combinations of microbes, for example, combination of bacterial cells and/or spores, used in compositions as provided herein, or to practice methods as provided herein, are fabricated as colonic or microflora-triggered delivery systems, as described for example, in Basit et al, J. Drug Targeting, 17:1, 64-71; Kotla, Int J Nanomedicine. 2016; 11:1089-1095; Bansai et al, Polim Med. 2014 April-June; 44 (2): 109-18; or, Shah et al, Expert Opin Drug Deliv. 2011 June; 8 (6): 779-96.

In alternative embodiments, combinations of microbes, for example, combination of bacterial cells and/or spores, used in compositions as provided herein, or to practice methods as provided herein, are encapsulated in at least one polymeric material, for example, a natural polymeric material, such that there is a core of bacterial cells and/or spores surrounded by a layer of the polymeric material, for example, a polysaccharide. Examples of suitable polymeric materials are those that have been demonstrated to remain intact through the GI tract until reaching the small or large intestine, where they are degraded by microbial enzymes in the intestines. Exemplary natural polymeric materials can include, but are not restricted to, chitosan, inulin, guar gum, xanthan gum, amylose, alginates, dextran, pectin, khava, and albizia gum (Dafe et al. (2017) Int J Biol Macromol 97:299-307; Kofla et al. (2016) Int J Nanomedicine 11:1089-1095).

In alternative embodiments, compositions provided herein are suitable for therapeutic administration to a human or other mammal in need thereof. In alternative embodiments the compositions are produced by a process comprising, for example: (a) obtaining fecal material from a mammalian donor subject, (b) subjecting the fecal material to at least one purification treatment under conditions that produce a single bacterial type population of bacteria and/or bacterial spores, or a combination of bacterial types and/or bacterial spores, (c) optionally combining the purified population with another purified population obtained from the same or different fecal material, from cultured conditions, or from a genetic stock center such as ATCC or DSMZ, (d) if the microbes, for example, bacterial cells, are not dormant, then treating the purified population(s) under conditions that cause vegetative bacterial cells to become dormant, and (e) placing the dormant bacteria and/or bacterial spores in a vehicle for administration.

In alternative embodiments, compositions, formulations and pharmaceutical compositions, which comprise on or a mixture of microbes (for example, bacteria) as provided herein, for example, bacterial cells and/or spores, or to practice methods as provided herein, are formulated for oral, topical, aerosol, rectal or gastric administration to a mammalian subject, for example, a human subject or individual in need thereof, such as a human infant or newborn.

In alternative embodiments, the compositions, formulations and pharmaceutical compositions are formulated for oral administration as a solid, semi-solid, gel or liquid form, such as in the form of a pill, tablet, capsule, lozenge, food, extract or beverage. In alternative embodiments, the compositions, formulations and pharmaceutical compositions are formulated for administration to an infant or newborn, for example, formulated with, mixed with or added to a liquid or powder including for example: milk (for example, human milk, cow's milk or soy protein, and optionally fortified with vitamins, minerals, and other nutrients), infant formula, soy-based formulas, amino acid-based formulas, hydrolyzed infant formula (made from cow's milk or soy protein that has been broken down into smaller proteins that are easier for infants to digest), supplemental (harvested) human mother's milk, and the like.

In alternative embodiments, the compositions, formulations and pharmaceutical compositions are formulated with, mixed with or added to a gel, liquid or powder or foods, for example, a food or gel that requires little mastication, such as any beverage, juices, juice extracts, yogurt, puddings, gelatins, and ice cream. Examples of extracts include crude and processed pomegranate juice, strawberry, raspberry and blackberry. Examples of suitable beverages include cold beverages, such as juices (pomegranate, raspberry, blackberry, blueberry, cranberry, acai, cloudberry, and the like, and combinations thereof) and teas (green, black, and the like) and oaked wine.

In alternative embodiments, formulations and pharmaceutical compositions further comprise, or methods as provided herein further comprise administration of, at least one prebiotic, metabolic precursor, drug or nutrient; optionally for example, the antibiotic comprises: a doxycycline, chlortetracycline, tetracycline hydrochloride, oxytetracycline, demeclocycline, methacycline, minocycline, penicillin, amoxicillin, erythromycin, vancomycin, clarithromycin, roxithromycin, azithromycin, spiramycin, oleandomycin, josamycin, kitasamycin, flurithromycin, nalidixic acid, oxolinic acid, norfloxacin, perfloxacin, amifloxacin, ofloxacin, ciprofloxacin, sparfloxacin, levofloxacin, rifabutin, rifampicin, rifapentine, sulfisoxazole, sulfamethoxazole, sulfadiazine, sulfadoxine, sulfasalazine, sulfaphenazole, dapsone, sulfapyridine, linezolid or any combination thereof. In alternative embodiments, the antibiotic or a combination of antibiotics are administered before, during and/or after administration of formulations and pharmaceutical compositions as provided herein.

### Gradual or Delayed Release Formulations

In alternative embodiments, exemplary compositions, formulations or pharmaceutical formulations as provided herein, or as used in methods as provided herein, comprise, contain or are coated by an enteric coating to protect a microbe, for example, a bacteria or mix of bacteria as provided herein, in a formulation and pharmaceutical compositions as provided herein to allow it to pass through the stomach and small intestine (for example, protect the administered combination of microbes such that a substantial majority of the microbes remain viable), although spores are typically resistant to the stomach and small intestines.

In alternative embodiments, compositions and formulations as provided herein, and compositions and formulations used to practice methods as provided herein, are formulated with a delayed release composition or formulation, coating or encapsulation. In alternative embodiments, compositions and formulations as provided herein, and compositions and formulations used to practice methods as provided herein, are designed or formulated for implantation of living microbes, for example, bacteria or spores, into the gut, including the intestine and/or the distal small bowel and/or the colon. In this embodiment the living microbes, for example, bacteria pass the areas of danger, for example, stomach acid and pancreatic enzymes and bile, and reach the intestine substantially undamaged to be viable and implanted in the GI tract.

In alternative embodiments, a formulation or pharmaceutical preparation, or the combination of microbes contained therein, is liquid, frozen, lyophilized or freeze-dried. In alternative embodiments, for example, for an encapsulated formulation, or are in powdered or aerosol or spray form. In alternative embodiments, if a formulation or pharmaceutical preparation as provided herein is in a powdered, lyophilate or freeze-dried form, the powder, lyophilate or freeze-dried form can be in a container such as a bottle, cartridge, packet or packette, or sachet, and the powder, lyophilate or freeze-dried form can be hydrated or reconstituted by a liquid, for example by adding water, saline, juice, milk, formula (such as infant formula) and the like to the powder, lyophilate or freeze-dried form, for example, the powdered, lyophilate or freeze-dried form can be added to the liquid. In alternative embodiments, a powdered, lyophilate or freeze-dried form as provided herein is in a bottle or container, and the liquid is added to the bottle or container, and this mixture can be consumed by an individual in need thereof. In alternative embodiments, a powdered, lyophilate or freeze-dried form as provided herein is in a cartridge that can be part of a container or bottle, and the powdered, lyophilate or freeze-dried form can be mixed with the liquid, for example, as described in U.S. Pat. No. 8,590,753. In alternative embodiments, a powdered, lyophilate or freeze-dried form as provided herein can be contained in or can be added to a container or bottle as described for example, in U.S. Pat. Nos. 10,315,815; 10,315,803; 10,281,317; 10,183,116; 9,809,374; 9,345,831; 9,173,999; 7,874,420.

In alternative embodiments, compositions and formulations as provided herein, and compositions and formulations used to practice methods as provided herein, are formulated for delayed or gradual enteric release using cellulose acetate (CA) and polyethylene glycol (PEG), for example, as described by Defang et al. (2005) Drug Develop. & Indust. Pharm. 31:677-685, who used CA and PEG with sodium carbonate in a wet granulation production process.

In alternative embodiments, compositions and formulations as provided herein, and compositions and formulations used to practice methods as provided herein, are formulated for delayed or gradual enteric release using a hydroxypropylmethylcellulose (HPMC), a microcrystalline cellulose (MCC) and magnesium stearate, as described for example, in Huang et al. (2004) European J. of Pharm. & Biopharm. 58:607-614).

In alternative embodiments, compositions and formulations as provided herein, and compositions and formulations used to practice methods as provided herein, are formulated for delayed or gradual enteric release using for example, a poly(meth)acrylate, for example a methacrylic acid copolymer B, a methyl methacrylate and/or a methacrylic acid ester, a polyvinylpyrrolidone (PVP) or a PVP-K90 and a EUDRAGIT® RL PO™, as described for example, in Kuksal et al. (2006) AAPS Pharm. 7 (1), article 1, E1 to E9.

In alternative embodiments, compositions and formulations as provided herein, and compositions and formulations used to practice methods as provided herein, are formulated for delayed or gradual enteric release as described in U.S. Pat. App. Pub. 20100239667. In alternative embodiments, the composition comprises a solid inner layer sandwiched between two outer layers. The solid inner layer can comprise the non-pathogenic bacteria and/or spores, and one or more disintegrants and/or exploding agents, or one or more effervescent agents or a mixture. Each outer layer can comprise a substantially water soluble and/or crystalline polymer or a mixture of substantially water soluble and/or crystalline polymers, for example, a polyglycol. These can be adjusted to achieve delivery of the living components to the intestine.

In alternative embodiments, compositions and formulations as provided herein, and compositions and formulations used to practice methods as provided herein, are formulated for delayed or gradual enteric release as described in U.S. Pat. App. Pub. 20120183612, which describes stable pharmaceutical formulations comprising active agents in a non-swellable diffusion matrix. In alternative embodiments, compositions and formulations as provided herein, and compositions and formulations used to practice methods as provided herein, are released from a matrix in a sustained, invariant and, if several active agents are present, independent manner and the matrix is determined with respect to its substantial release characteristics by ethylcellulose and at least one fatty alcohol to deliver bacteria distally.

In alternative embodiments, compositions and formulations as provided herein, and compositions and formulations used to practice methods as provided herein, are formulated for delayed or gradual enteric release as described in U.S. Pat. No. 6,284,274, which describes a bilayer tablet containing an active agent (for example, an opiate analgesic), a polyalkylene oxide, a polyvinylpyrrolidone and a lubricant in the first layer and a second osmotic push layer containing polyethylene oxide or carboxy-methylcellulose.

In alternative embodiments, compositions and formulations as provided herein, and compositions and formulations used to practice methods as provided herein, are formulated for delayed or gradual enteric release as described in U.S. Pat. App. Pub. No. 20030092724, which describes sustained release dosage forms in which a nonopioid analgesic and opioid analgesic are combined in a sustained release layer and in an immediate release layer, sustained release formulations comprising microcrystalline cellulose, EUDRAGIT RSPO™, CAB-O-SIL™, sodium lauryl sulfate, povidone and magnesium stearate.

In alternative embodiments, compositions and formulations as provided herein, and compositions and formulations used to practice methods as provided herein, are formulated for delayed or gradual enteric release as described in U.S. Pat. App. Pub. 20080299197, describing a multi-layered tablet for a triple combination release of active agents to an environment of use, for example, in the GI tract. In alternative embodiments, a multi-layered tablet is used, and it can comprise two external drug-containing layers in stacked arrangement with respect to and on opposite sides of an oral dosage form that provides a triple combination release of at least one active agent. In one embodiment the dosage form is an osmotic device, or a gastro-resistant coated core, or a matrix tablet, or a hard capsule. In these alternative embodiments, the external layers may contain biofilm dissolving agents and internal layers can comprise viable/living bacteria, for example, a formulation comprising: one (for example, as in a synbiotic, or combination of one species and a probiotic, such as a synbiotic combination as set forth in Table 8 or Table 32), or, at least two different species or genera (or types) of, non-pathogenic bacteria as used to practice methods as provided herein.

In alternative embodiments, compositions and formulations as provided herein, and compositions and formulations used to practice methods as provided herein, are formulated as multiple layer tablet forms, for example, where a first layer provides an immediate release of a formulation or pharmaceutical preparation as provided herein and a second layer provides a controlled-release of another (or the same) bacteria or drug, or another active agent, for example, as described for example, in U.S. Pat. No. 6,514,531 (disclosing a coated trilayer immediate/prolonged release tablet), U.S. Pat. No. 6,087,386 (disclosing a trilayer tablet), U.S. Pat. No. 5,213,807 (disclosing an oral trilayer tablet with a core comprising an active agent and an intermediate coating comprising a substantially impervious/impermeable material to the passage of the first active agent), and U.S. Pat. No. 6,926,907 (disclosing a trilayer tablet that separates a first active agent contained in a film coat from a core comprising a controlled-release second active agent formulated using excipients which control the drug release, the film coat can be an enteric coating configured to delay the release of the active agent until the dosage form reaches an environment where the pH is above four).

In alternative embodiments, compositions and formulations as provided herein, and compositions and formulations used to practice methods as provided herein, are formulated for delayed or gradual enteric release as described in U.S. Pat. App. Pub. 20120064133, which describes a release-retarding matrix material such as: an acrylic polymer, a cellulose, a wax, a fatty acid, shellac, zein, hydrogenated vegetable oil, hydrogenated castor oil, polyvinylpyrrolidone, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a copolymer of lactic and glycolic acid, carboxymethyl starch, potassium methacrylate/divinylbenzene copolymer, cross linked polyvinylpyrrolidone, polyvinyl alcohols, polyvinyl alcohol copolymers, polyethylene glycols, non-cross linked polyvinylpyrrolidone, polyvinyl acetates, polyvinyl acetate copolymers or any combination thereof. In alternative embodiments, spherical pellets are prepared using an extrusion/spheronization technique, of which many are well known in the pharmaceutical art. The pellets can comprise one or more formulations or pharmaceutical preparations as provided herein.

In alternative embodiments, compositions and formulations as provided herein, and compositions and formulations used to practice methods as provided herein, are formulated for delayed release, extended release, or gradual enteric release, for example, as described in U.S. Pat. App. Pub. 20110218216, which describes an extended-release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropyl cellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candelilla wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, cresylic acid or malic acid or a mixture thereof.

In alternative embodiments, therapeutic combinations or formulations, or pharmaceuticals or the pharmaceutical preparations as provided herein, or as used in methods as provided herein, are formulated as a delayed or gradual enteric release composition or formulation, and optionally the formulation comprises a gastro-resistant coating designed to dissolve at a pH of 7 in the terminal ileum, for example, an active ingredient is coated with an acrylic based resin or equivalent, for example, a poly(meth)acrylate, for example a methacrylic acid copolymer B, NF, which dissolves at pH 7 or greater, for example, comprises a multimatrix (MMX) formulation. In alternative embodiments, compositions and formulations as provided herein, and compositions and formulations used to practice methods as provided herein, are powders or aerosol that can be included into a suitable carrier, for example, such as a liquid, a tablet or a suppository. In alternative embodiments, compositions and formulations as provided herein, and compositions and formulations used to practice methods as provided herein, are ‘powders for reconstitution’ as a liquid to be drunk, placed down a naso-duodenal tube or used as an enema for patients to take home and self-administer enemas. In alternative embodiments, compositions and formulations as provided herein, and compositions and formulations used to practice methods as provided herein, are micro-encapsulated, formed into tablets and/or placed into capsules, especially enteric-coated capsules. In alternative embodiments, compositions as provided herein are formulated to be effective in a given mammalian subject in a single administration or over multiple administrations. In some embodiments, a substrate or prebiotic required by the bacterial type in a formulation as provided herein is administered for a period of time in advance of the administration of the combination of microbes, for example, bacterial compositions, as provided herein. Such administration (for example, of prebiotics) pre-loads the gastrointestinal tract with the substrates needed by the bacterial types of the composition and increases the potential for the bacterial composition to have adequate resources to perform the required metabolic reactions. In other embodiments, the composition is administered simultaneously with the substrates required by the bacterial types a formulation as provided herein. In still other embodiments the substrate or prebiotic is administered alone. In alternative embodiments, efficacy is measured by an increase in the population of those bacterial types in the subject's intestinal tract, or an increase in the population of those bacterial types originally found in the subject's intestinal tract before treatment.

In alternative embodiments, compositions as provided herein comprise, further comprise, or have added to: at least one probiotic or prebiotic, wherein optionally the prebiotic comprises an inulin, lactulose, extracts of artichoke, chicory root, oats, barley, various legumes, garlic, kale, beans or flakes or an herb, mammalian milk oligosaccharides, or mucin, wherein optionally the probiotic comprises a cultured or stool-extracted microorganism or bacteria, or a bacterial component, and optionally the bacteria or bacterial component comprises or is derived from a Bacteroidetes, a Firmicutes, a Proteobacteria, a Verucomicrobia, an Actinobacteria, a Lactobacilli, a Bifidobacteria, an E. coli, a Streptococcus faecalis and equivalents.

In alternative embodiments, compositions as provided herein comprise, further comprise, or have added to: at least one congealing agent, wherein optionally the congealing agent comprises an arrowroot or a plant starch, a powdered flour, a powdered potato or potato starch, an absorbent polymer, an Absorbable Modified Polymer, and/or a corn flour or a corn starch; or, further comprise an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient; or, further comprise, or have added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides-cathelicidin-derived peptides, small lytic peptide, PTP-7, nitric oxide, neo-emulsions; ozone, lytic bacteriophages, lactoferrin, xylitol hydrogel, synthetic iron chelators, a statin (optionally lovastatin (optionally MEVACOR™), simvastatin (optionally ZOCOR™), atorvastatin (optionally LIPITOR™), pravastatin (optionally PRAVACHOL™), fluvastain (optionally LESCOL™) or rosuvastatin (optionally CRESTOR™)), cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

In alternative embodiments, compositions as provided herein comprise, further comprise, or have added to: a flavoring or a sweetening agent, an aspartame, a stevia, monk fruit, a sucralose, a saccharin, a cyclamate, a xylitol, a vanilla, an artificial vanilla or chocolate or strawberry flavor, an artificial chocolate essence, or a mixture or combination thereof.

### Products of Manufacture and Kits

Provided are products of manufacture, for example, implants or pharmaceuticals, and kits, containing components for practicing methods as provided herein, for example, including a formulation comprising a combination of microbes as provided herein, such as for example, freshly isolated microbes, cultured microbes, or genetically engineered microbes, or one (for example, as in a synbiotic, or combination of one species and a probiotic, such as a synbiotic combination as set forth in Table 8 or Table 32), or, at least two different species or genera (or types) of, non-pathogenic bacteria, wherein each of the non-pathogenic bacteria comprise (or are in the form of) a plurality of non-pathogenic colony forming live bacteria, a plurality of non-pathogenic germinable bacterial spores, or a combination thereof, and optionally including instructions for practicing methods as provided herein.

### Companion Diagnostics and Participant Biomarkers

Provided are biomarkers indicative of dysbiosis or eubiosis in adults that are at high risk for a disease such as colorectal cancer. These biomarkers may be in the form of microbial species abundance in the gut (or abundance in the colon), microbial gene expression or protein expression, or abundance of a metabolite in a stool sample or a sample of bacteria taken from the gut. Alternatively, the biomarkers may be metabolite concentration, cytokine profile, or protein expression in the blood. These biomarkers are used to determine the level of dysbiosis in a participants gut and predict methods of treatment that will improve the dysbiosis to reduce the risk associated with disease, such as colorectal cancer.

### Genetic Modification of Microbial Therapeutics

In alternative embodiments, microbes, for example, bacteria or mixes of bacteria, used in compositions as provided herein, or used to practice methods as provided herein, are genetically engineered (or genetically modified). In alternative embodiments, one several (for example, between about 1% and 99%) or all of a combination or mix of microbes as provided herein, or used to practice methods as provided herein, are genetically engineered.

In alternative embodiments, microbes, for example, bacteria or mixes of bacteria, used in compositions as provided herein, or used to practice methods as provided herein, are genetically engineered to metabolize or consume a prebiotic, for example, a prebiotic as described in Table 3.

In alternative embodiments, microbes, for example, bacteria or mixes of bacteria, used in compositions as provided herein, or used to practice methods as provided herein, are genetically engineered to increase their efficacy, for example, to increase the efficacy of a chemotherapy, a radiation therapy, an immune checkpoint inhibitor (for example, a checkpoint inhibitor therapy), a Chimeric Antigen Receptor (CAR) T-cell therapy (CAR-T) or other immunotherapy or cancer treatment.

In alternative embodiments, microbes, for example, bacteria or mixes of bacteria, used in compositions as provided herein, or used to practice methods as provided herein, are genetically engineered to substantially decrease, reduce or eliminate their toxicity.

In alternative embodiments, microbes, for example, bacteria or mixes of bacteria, used in compositions as provided herein, or used to practice methods as provided herein, are genetically engineered to comprise a kill switch so they can be rendered non-vital after administration of an appropriate trigger or signal.

In alternative embodiments, microbes, for example, bacteria or mixes of bacteria, used in compositions as provided herein, or used to practice methods as provided herein, are genetically engineered to secrete anti-inflammatory compositions or have an anti-inflammatory effect.

In alternative embodiments, microbes, for example, bacteria or mixes of bacteria, used in compositions as provided herein, or used to practice methods as provided herein, are genetically engineered to secrete an anti-cancer or a cytostatic substance.

Microbes, for example, bacteria, used in compositions as provided herein, or used to practice methods as provided herein, can be genetically engineered using any method known in the art, for example, as discussed in the Examples, below. For example, one or more gene sequence(s) and/or gene cassette(s) may be expressed on a high-copy plasmid, a low-copy plasmid, or a chromosome. In some embodiments, expression from the plasmid is used to increase expression of an inserted, for example, heterologous nucleic acid, for example, a gene or protein encoding sequence or an inhibitory nucleic acid such as an antisense or siRNA-encoding nucleic acid. The inserted nucleic acid of interest can be inserted into a bacterial chromosome at one or more integration sites.

For example, in alternative embodiments, microbes are genetically engineered to comprise one or more gene sequence(s) and/or gene cassette(s) for producing a non-native anti-inflammation and/or gut barrier function enhancer molecule. In alternative embodiments, the anti-inflammation and/or gut barrier function enhancer molecule comprises a short-chain fatty acid, butyrate, propionate, acetate, IL-2, IL-22, superoxide dismutase (SOD), GLP-2, GLP-1, IL-10, IL-27, TGF-.beta.1, TGF-.beta.2, N-acyl phosphatidylethanolamines (NAPES), elafin (also known as peptidase inhibitor 3 or SKALP), trefoil factor, melatonin, PGD2, kynurenic acid, and kynurenine. A molecule may be primarily anti-inflammatory, for example, IL-10, or primarily gut barrier function enhancing, for example, GLP-2. In alternative embodiments, microbes are genetically engineered to comprise one or more gene sequence(s) and/or gene cassette(s) that are inhibitory to the activity of, or substantially or completely inhibit expression of, bacterial virulence factors, toxins, or antibiotic resistance functions.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, “and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

The following Examples describe methods and compositions for practicing embodiments as provided herein, including methods for making and using compositions comprising non-pathogenic bacteria and non-pathogenic germinable bacterial spores used to practice methods as provided herein.

### Example 1: Anaerobic Culture Conditions

### Preparation of Anaerobic Growth Medium

Exemplary bacterial strains described herein are obligate anaerobes that require anaerobic conditions for culture. Growth media suitable for culture of anaerobic bacteria include reducing agents such as L-cysteine, sodium thioglycolate, and dithiothreitol, for the purpose of scavenging and removing oxygen. Appropriate commercially available anaerobic growth media include but are not limited to ANAEROBE BASAL BROTH™ (OXOID/THERMO SCIENTIFIC™), REINFORCED CLOSTRIDIAL MEDIUM™ (OXOID/THERMO SCIENTIFIC™), WILKINS-CHALGREN ANAEROBE BROTH™ (OXOID/THERMO SCIENTIFIC™), SCHAEDLER ANAEROBE BROTH™ (OXOID/THERMO SCIENTIFIC™), and BRAIN HEART INFUSION BROTH™ (OXOID/THERMO SCIENTIFIC™). Animal free medium for anaerobic culture include but are not limited to VEGITONE ACTINOMYCES BROTH™ (MILLIPORE-SIGMA™), MRS BROTH™ (MILLIPORE-SIGMA™), VEGITONE INFUSION BROTH™ (MILLIPORE-SIGMA™), and VEGITONE CASEIN SOYA BROTH™ (MILLIPORE-SIGMA™).

One liter of Anaerobic growth medium is prepared by combining the manufacturer's recommended amount in grams of dry growth medium powder with 800 ml Reagent Grade Water (NERL™) along with 1 ml 2.5 mg/ml resazurin (ACROS Organics™) in a 2 liter beaker and stirred on a heated stir plate until dissolved. The volume is adjusted to 1 liter by addition of additional Reagent Grade Water, then the volume is brought to a boil while stirring until the red color imbued by the resazurin becomes colorless, indicating removal of oxygen from the solution. The volume is then removed from the stir plate to cool for 10 minutes on the benchtop before further manipulation.

From the 1-liter volume, 900 ml is transferred to a 1 liter anaerobic media bottle (CHEMGLASS LIFE SCIENCES™) and then placed back on the heated stir plate to remove any oxygen introduced in the transfer, as indicated by the color of the added resazurin. The anaerobic media bottle is then stoppered with a butyl rubber bung that is secured by a crimped aluminum collar, and then brought into the anaerobic chamber (COY LAB TYPE A VINYL ANAEROBIC CHAMBER™, COY LABORATORY PRODUCTS™, Grass Lake, MI). The butyl rubber bung is removed to open the bottle within the anaerobic chamber to equilibrate with the anoxic atmosphere while cooling to ambient temperature. The bottle is resealed with a fresh butyl rubber bung and crimped aluminum collar, brought out of the chamber, then sterilized by autoclaving for 20 minutes followed by slow exhaust.

Alternatively, the 1-liter volume can be aliquoted into smaller 50 ml volumes in 100 ml serum bottles (CHEMGLASS LIFE SCIENCES™, Vineland New Jersey). The boiled 1-liter volume is transferred to a one-liter screw cap bottle, which is placed back on the heated stir plate to drive off any oxygen introduced by the transfer. The bottle cap is then securely tightened, and the bottle is immediately brought into the anaerobic chamber, where the cap is loosened to allow the volume to equilibrate with the anoxic atmosphere and to cool for 1 hour. The volume is then transferred in 50 ml aliquots to 100 ml serum bottles using a serological pipette, then the liquid contents cooled to ambient temperature. The bottles are sealed with butyl rubber bungs and crimped aluminum collars, brought out of the chamber, then sterilized by autoclaving for 20 minutes followed by slow exhaust.

Alternatively, the 1-liter volume can be aliquoted into smaller 10 ml volumes in sealed Hungate tubes (CHEMGLASS LIFE SCIENCES™, Vineland New Jersey) as follows: The boiled 1-liter volume is transferred to a one-liter screw cap bottle, which is placed back on the heated stir plate to drive off any oxygen introduced by the transfer. The bottle cap is then securely tightened, and the bottle is immediately brought into the anaerobic chamber, where the cap is loosened to allow the volume to equilibrate with the anoxic atmosphere and to cool for 1 hour. The volume is then transferred in 10 ml aliquots to fill racked Hungate tubes, then allowed to cool to ambient temperature, followed by securely capping and sealing each tube with screw caps with butyl rubber septa. The sealed Hungate tube aliquots are removed from the anaerobic chamber and then sterilized by autoclaving for 20 minutes followed by slow exhaust.

Alternatively, the 1 liter volume can be combined with 15 grams Agar (THERMO SCIENTIFIC™) to make solid media in culture plates as follows: The boiled 1 liter volume is poured into a 1 liter screw cap bottle, followed by replacement on a heated stir plate to remove any oxygen introduced by the transfer as indicated by the colorless resazurin oxygen indicator. The bottle is loosely capped and then autoclaved for 20 minutes followed by slow exhaust. Immediately after autoclaving, the cap of the bottle is tightened prior to bringing the bottle into the anaerobic chamber. Once in the anaerobic chamber, the cap is loosened and the contents cooled for 30 minutes, then 25 ml volumes are poured into culture plates and allowed to cool until solidified. The plates are then allowed to dry in the anaerobic chamber for 24 hours prior to use.

### Live Cryostorage of Anaerobic Microbes

Individual microbes of interest are prepared for long-term cryogenic live storage by inoculating a pure colony isolate grown on anaerobic solid medium into a prepared Hungate tube containing liquid anaerobic growth medium previously determined to be optimal for the species. The inoculated Hungate tube is then incubated at 37° C. until turbidity evidence of exponential growth is observed. The Hungate culture is brought into the anaerobic chamber, and 1 ml is transferred by pipette into a 2 ml screw cap cryotube containing anoxic 1 ml Biobank Buffer (Phosphate Buffered Saline (PBS) plus 2% trehalose plus 10% dimethyl sulfoxide, filter sterilized and bubbled with nitrogen gas to remove oxygen). The resulting 2 ml volume is thoroughly mixed by pipetting, securely tightened, then placed for long-term storage in the gaseous phase of a liquid nitrogen Dewar or in a −80° C. freezer.

Microbes in fecal matter can be cryogenically preserved for later revival and new strain discovery as follows. Freshly obtained fecal material is brought into the anaerobic chamber and 1 gram is weighed and mixed in a 15 ml conical tube with a solution consisting of 5 ml Anaerobe Basal Broth (ABB) and 5 ml BIOBANK BUFFER™. The tube is tightly capped, and the fecal matter is thoroughly suspended in the solution by vortexing for 20 minutes, followed by incubation upright on ice to allow large particles to settle. One ml aliquots of the fecal suspension are then transferred by pipette to a 2 ml screw cap cryotube, securely tightened, then placed for long-term storage in the gaseous phase of a liquid nitrogen Dewar or in a −80° C. freezer.

### Example 2: Fecal Matter Collection and Processing

### Infant Stool Sample Collection

Fecal matter donations are acquired from infants aged 1 month to 3 years. If from the US, donor infants are representative of the US statistics for birth mode (C-section/Vaginal) and feeding mode (Breast/Mixed/Formula) as well as the racial and ethnic demographics of the United States. Donor infants are screened for antibiotic use prior to donation.

Donors receive a stool sampling kit by mail sent to the contact address provided. Stool samples are collected by the subject at home. Stool sampling kits consist of the following: gloves, instructions for stool collection, welcome card, freezer pack, Styrofoam container, plastic scoop for fecal collection, a DNA/RNA preservative tube for immediate sample preservation, FEDEX™ shipping labels, and stickers to seal kit prior to shipping. Subjects receive a freezer pack for chilling the samples and are instructed to place it in their freezer overnight upon receipt of the sampling kit. The stool sampling kit also includes a plastic scoop so that fecal samples can be retrieved directly from the diaper. The subject is instructed to use the scoop to collect the fecal sample as soon as possible after the sample is produced with the primary scoop and to use the secondary scoop provided with the DNA/RNA preservative tube to collect what remains on the diaper. Subjects are instructed to wear the gloves provided in the kit before scooping the fecal sample. The subject is instructed to seal the plastic container inside a specimen bag and remove gloves. The subject is then instructed to remove the ice pack from their home freezer and place it inside the Styrofoam cooler box along with the bagged and sealed stool sample. The subject is instructed to close the lid on the foam container and then close the box, sealing with the packing sticker. The subject is instructed to schedule a FEDEX™ pickup at their home within 24 hours of stool collection or drop it off at the nearest FEDEX™ location. Under these conditions the stool has been demonstrated to remain chilled during shipment for as long as 48 hours.

Once received, the stool sample receptacle is given a unique alphanumeric identifier that is used subsequently for sample tracking. The stool is unpacked from the shipping box in a laboratory setting and the temperature evaluated to ensure the sample is preserved appropriately. The sample is then homogenized and divided into enough individual aliquots for all projected analyses prior to freezing and storage at −80° C., as described below. The RNA preservative aliquot is stored at −20° C. upon arrival until further use. All aliquots also bear an alphanumeric identifier corresponding to the subject donor. Any remaining stool after the aliquots are taken is disposed of as biohazardous waste.

### Preparation of Infant Fecal Matter Samples for Analysis

Fecal matter received from donors can be processed using any method known in the art, for example, as described in U.S. Pat. Nos. 10,493,111; 10,471,107; 10,286,012; 10,314,863; 9,623,056.

For example, received fecal matter in its receptacle is placed on ice and then brought into the anaerobic chamber. The receptacle is opened, and the sample is diluted 1:1 with anoxic PBS. The mixture is homogenized by hand or in the case of sufficient sample size, with a blender cup to a smooth consistency.

The homogenized fecal matter is then processed and aliquoted for cryopreservation for several different analyses as follows:
1) Live Cryopreservation for Fecal Microbiome Transfer (FMT) Experiments in Mice: Homogenized fecal matter is combined with FMT Buffer (Phosphate Buffered Saline plus 1% L-Cysteine plus 2% Trehalose plus 30% glycerol). The tube is then vortexed for 20 seconds and then placed on ice. A pipette is used to transfer 1 ml aliquots into 2 ml cryotubes that are then tightly capped. Aliquoted samples are frozen and then stored at −80° C.
2) Live Cryopreservation for Isolation and Discovery of Microbes: Homogenized fecal matter is combined in a conical tube with Anaerobe Basal Broth and Biobank Buffer (Phosphate Buffered Saline plus 2% Trehalose plus 10% dimethyl sulfoxide), tightly capped and vortexed for 20 seconds, then put on ice upright and allowed to settle for 10 minutes. Using a pipette, 1 ml aliquots are added to 2 ml cryotubes, which are then tightly capped. Aliquoted samples are frozen and then stored at −80° C.
3) For Genomic, Metabolomic Analyses, and Immune Phenotyping: Homogenized fecal matter is aliquoted in 1 ml volumes into 2 ml cryo tubes. Aliquoted samples are frozen and then stored at −80° C.
   In addition to the homogenized fecal sample, raw fecal sample is used to evaluate the pH of the sample.

### Adult Stool Sample Collection

Fecal matter donations are acquired from healthy volunteers as well as individuals exhibiting disease symptoms. Donors can be cancer patients being administered approved therapies or participating in clinical trials testing various cancer treatment regimens. Donors can be healthy volunteers that do not exhibit disease symptoms, or are at risk for disease based on family history or prior diagnostic findings.

Donors receive a stool sampling kit by mail sent to the contact address provided or by their physician. Stool samples are collected by the subject at home, or with necessary assistance if hospitalized. Stool sampling kits consist of the following: gloves, instructions for stool collection, welcome card, freezer pack, Styrofoam container, plastic bracket and plastic commode to aid in stool collection, FedEx shipping labels, and stickers to seal kit prior to shipping. Subjects receive a freezer pack for chilling the samples and are instructed to place it in their freezer overnight upon receipt of the sampling kit. The stool sampling kit also includes a plastic commode that can be placed safely and securely on a toilet seat, allowing the subject to defecate directly into a plastic container. The subject is instructed to use the commode to capture a stool sample, then seal the sample container with a provided snap-cap lid. Subjects are instructed to wear the gloves provided in the kit before removing the sample container from the toilet. The subject is instructed to seal the plastic container inside a specimen bag and remove gloves. The subject is then instructed to remove the ice pack from their home freezer and place it inside the Styrofoam cooler box along with the bagged and sealed stool sample. The subject is instructed to close the lid on the foam container and then close the box, sealing with the packing sticker. The subject is instructed to schedule a FedEx pickup at their home within 24 hours of stool collection or drop it off at the nearest FedEx location. Under these conditions the stool has been demonstrated to remain chilled during shipment for as long as 48 hours.

Once received, the stool sample receptacle is given a unique alphanumeric identifier that is used subsequently for sample tracking. The stool is unpacked from the shipping box in a laboratory setting, homogenized, and divided into enough individual aliquots for all projected analyses prior to freezing and storage at −80° C., as described below. All aliquots also bear an alphanumeric identifier corresponding to the subject donor. Any remaining stool after the aliquots are taken is disposed of as biohazardous waste.

### Preparation of Adult Fecal Matter Samples for Analysis

Fecal matter received from donors can be processed using any method known in the art, for example, as described in U.S. Pat. Nos. 10,493,111; 10,471,107; 10,286,012; 10,314,863; 9,623,056.

For example, received fecal matter in its receptacle is placed on ice and then brought into the anaerobic chamber. The receptacle is opened and approximately 40 g stool is weighed into a tared specimen cup. 15 ml sterile anoxic PBS is then added, and the mixture is homogenized by a hand-held homogenizer to achieve a smooth consistency.

The homogenized fecal matter is then processed and aliquoted for cryopreservation for several different analyses as follows:
1) For Genomic and Transcriptomic Analyses: homogenized fecal matter is weighed and then an equal volume to weight amount of RNALATER® (RNAlater®) (THERMO FISHER SCIENTIFIC™) solution is added. The tube is capped tightly and then vortexed for 20 seconds and then placed on ice. A pipette is used to transfer 1 ml aliquots into 2 ml Eppendorf tubes. Aliquoted samples are frozen on dry ice and then stored at −80° C.
2) Live Cryopreservation for Fecal Microbiome Transfer (FMT) Experiments in Mice: Homogenized fecal matter is combined with FMT Buffer (Phosphate Buffered Saline plus 1% L-Cysteine plus 2% Trehalose plus 30% glycerol). The tube is then vortexed for 20 seconds and then placed on ice. A pipette is used to transfer 1 ml aliquots into 2 ml cryotubes that are then tightly capped. Aliquoted samples are frozen on dry ice and then stored at −80° C.
3) Live Cryopreservation for Isolation and Discovery of Microbes: Homogenized fecal matter is combined in a conical tube with Anaerobe Basal Broth and Biobank Buffer (Phosphate Buffered Saline plus 2% Trehalose plus 10% dimethyl sulfoxide), tightly capped and vortexed for 20 seconds, then put on ice upright and allowed to settle for 10 minutes. Using a pipette, 1 ml aliquots are added to 2 ml cryotubes, which are then tightly capped. Aliquoted samples are frozen on dry ice and then stored at −80° C.
4) For Genomic, Metabolomic Analyses, and Immune Phenotyping: Homogenized fecal matter is aliquoted in 1 ml volumes into 2 ml cryo tubes. Aliquoted samples are frozen and then stored at −80° C.

### Example 3: Patient Data Collection from Infant Clinical Trials and Data Analysis on the Same

### MY BABY BIOME™ Clinical Study

The MY BABY BIOME™ Clinical Study (NCT05472688) is a study designed to evaluate the diversity of the gut microbiome among healthy infants in the US. Samples were collected from over 400 infants between the age of four and ten weeks, when immune development is extremely critical, and were evaluated via metagenomics, metabolomics, and proteomics to determine key distinguishing biomarkers. To ensure an accurate understanding of the infant gut in the United States, participants were sampled from different birth (vaginal versus (vs)C-section) and feeding (formula, breast fed, or mixed) modes with a population that represents the racial, ethnic, and geographic diversity of the US population.

### Whole Genome Sequencing of Infant Fecal Samples

Aliquots of homogenized fecal matter are thawed and subjected to centrifugation for 20 minutes at 6000 g to pellet the cells. After centrifugation, 0.8 ml supernatant is carefully removed by pipette, leaving 0.1 ml pellet and medium for gDNA processing. Total genomic DNA is extracted from the cell pellet using the MAGATTRACT POWERMICROBIOME™ DNA/RNA EP kit (Qiagen). Genomic DNA is then prepared for Whole Genome Sequencing analysis using the KAPA LIBRARY PREP™ kit (Roche). Sequencing analysis is conducted on the Illumina platform using paired-end 150 bp reads.

Sequencing data is first processed to remove low quality reads and adapter contamination using TRIM GALORE™ (Babraham Bioinformatics, Cambridge, UK), a wrapper for CUTADAPT™, a tool for quality control of high-throughput sequencing reads.

Microbial and archaeal assembled genomes from the Genome Taxonomy Database (GTDB) (Parks et al. (2019) bioRxiv 771964, Méric et al. (2019) bioRxiv 712166) were used as a reference for classification using CENTRIFUGE™ (Kim et al. (2016) Genome Research 26:1721-1729). CENTRIFUGE™ classifies sequencing reads from a metagenomic fecal sample to reference sequences and uses an expectation-maximization method to estimate relative abundance of the taxa present in the sample.

A second classification was performed using a custom built gut bacteria specific index and alternate classification algorithms. The custom index was constructed in multiple steps. First, 1085 gut and oral genera were identified through Unified Human Gastrointestinal Genome (UHGG) (Almeida et al. (2021) Nature Biotechnology 39:105-114). Second, 132,128 bacterial genome assemblies for the identified gut and oral genera were batch downloaded from NCBI. Next, the downloaded assemblies were clustered and dereplicated using METAGENOMICS-INDEX CORRECTION™ software (https://github.com/rrwick/Metagenomics-Index-Correction) with a threshold of 0.01. NCBI taxonomy naming was replaced with GTDB taxonomy to maintain consistent interpretation with the primary classification. From the dereplicated NCBI assemblies we built an index for use with the KRAKEN2™ (CCB, Johns Hopkins University) metagenomic classification package (Wood et al. (2019) Genome Biology 20:257). Mock communities were simulated and used to validate this classification method and showed increased accuracy in classification on the subspecies level.

### Analysis of Whole Genome Sequencing of Infant Fecal Samples

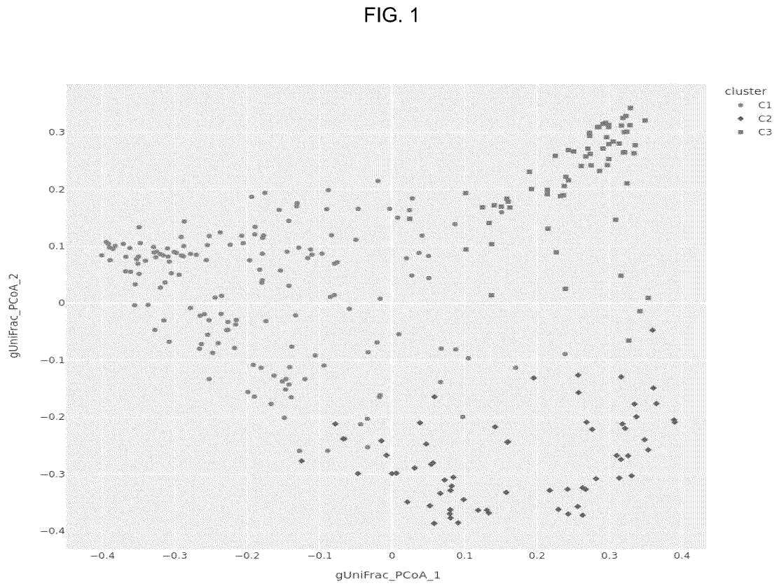

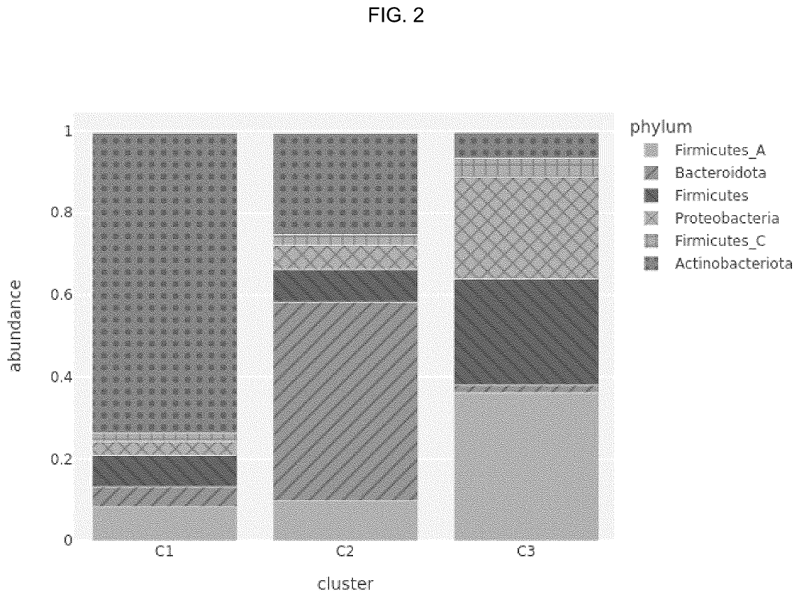

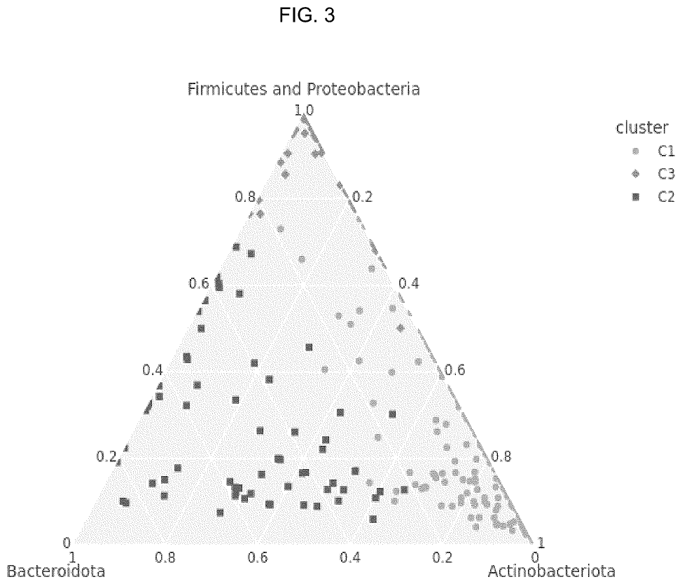

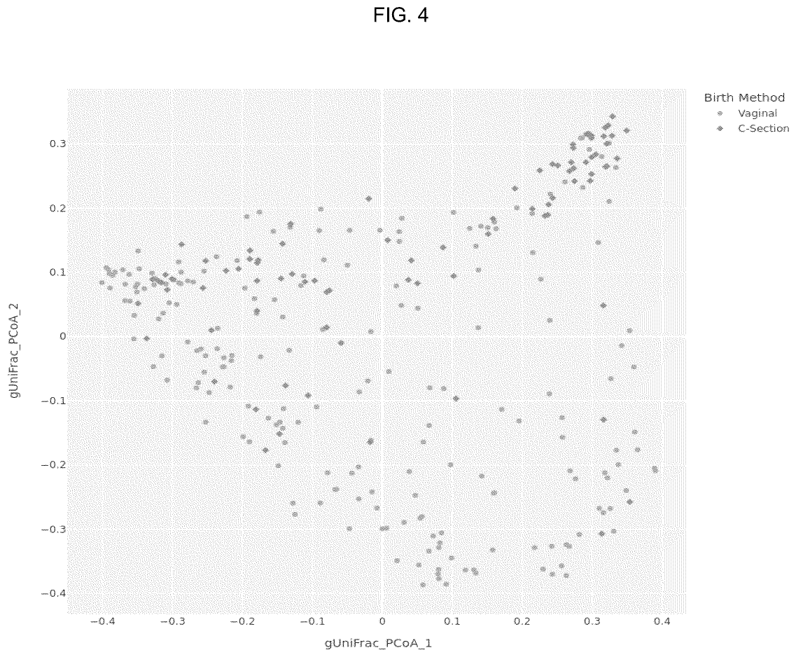

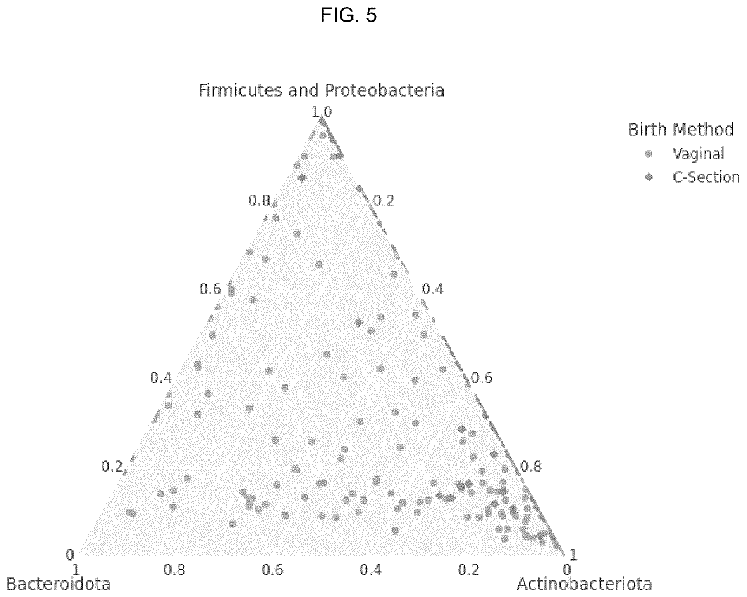

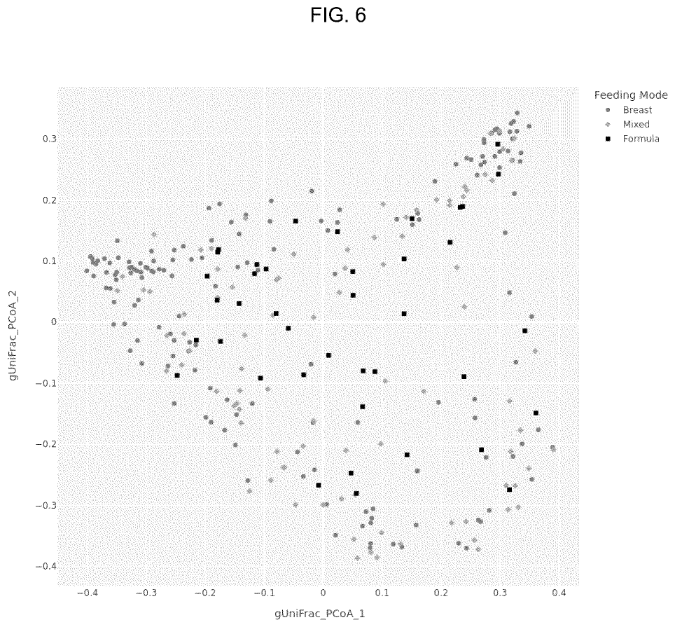

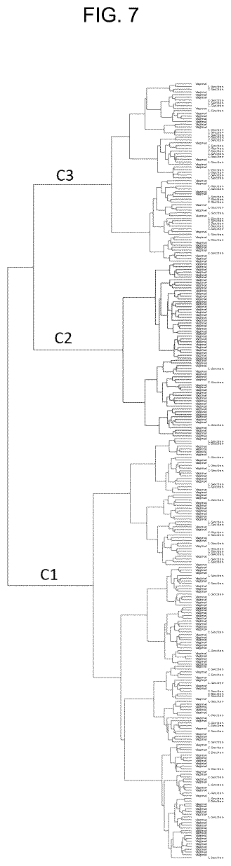

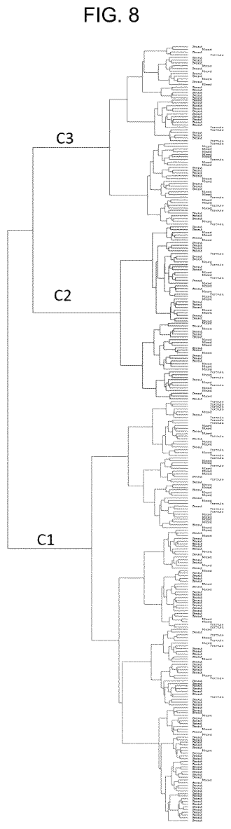

The metagenomes were broadly grouped into 3 clusters based on the microbial composition using GUNIFRAC™ (GUNIFRAC™, GITHUB™, San Francisco, CA) (Chen et al. (2012) Bioinformatics 28:2106-2113) to measure distances between samples, and the Ward method of agglomerative clustering. The relationship between samples is shown by principal coordinate analysis (FIG. 1) and the compositional differences between clusters with bar plots (FIG. 2). Specifically, one cluster (referred to as C1) is extremely high in the phylum Actinobacteriota. Another cluster is dominated by Bacteroidota (C2), while the third (C3) is enriched in Firmicutes and Proteobacteria (FIG. 2, FIG. 3). While C1 contains samples from both vaginal and C-section birth infants, C2 is almost exclusively vaginal birth and C3 is enriched in C-section infants (FIG. 4 and FIG. 5) (chi-squared p-value for birth mode associations with GUNIFRAC™ clusters is less than 0.0001). C1 is also depleted in exclusively formula-fed infants (FIG. 6) (chi-squared p-value for the feeding mode associations with gUniFrac clusters is 0.05). These trends can also be seen in dendrograms, where the Ward method of agglomerative clustering on GUNIFRAC™ sample to sample similarities shows how samples cluster according to microbiome composition (FIG. 7 and FIG. 8). Clusters C1, C2, and C3 form three distinct branches of the dendrogram.

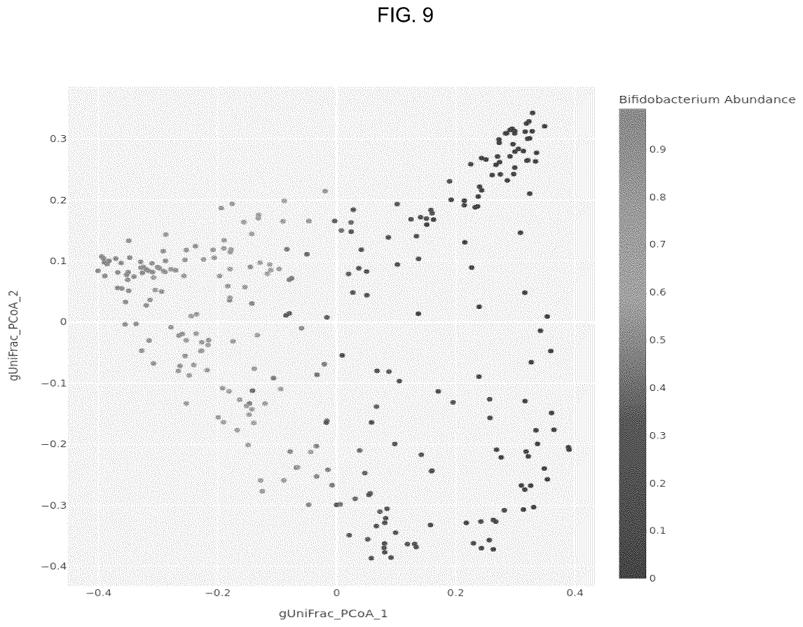

The high abundance of Actinobacteriota in C1 is driven almost exclusively by the genus Bifidobacterium (FIG. 9). Based on historical populations, metabolic output, and the presence of pathogens, it can be inferred that the cluster enriched in Bifidobacterium represents eubiosis for the infants, while the other clusters represent two unique dysbioses.

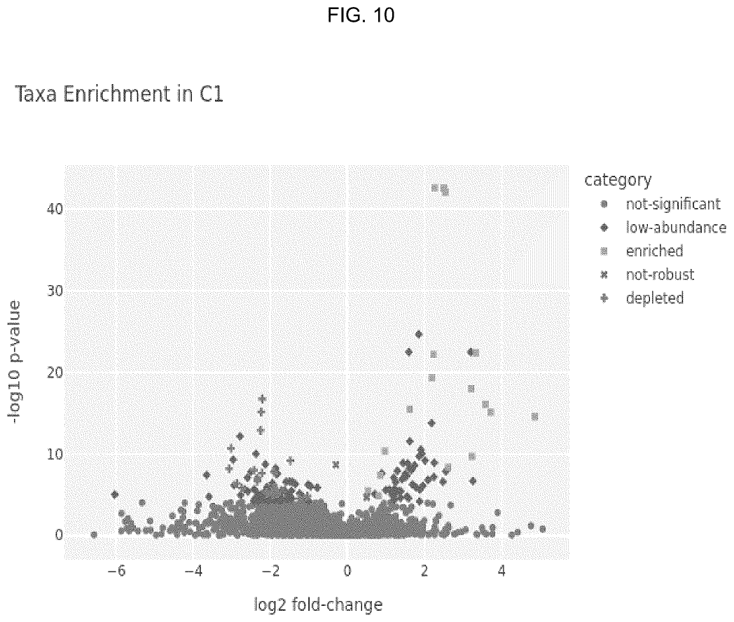

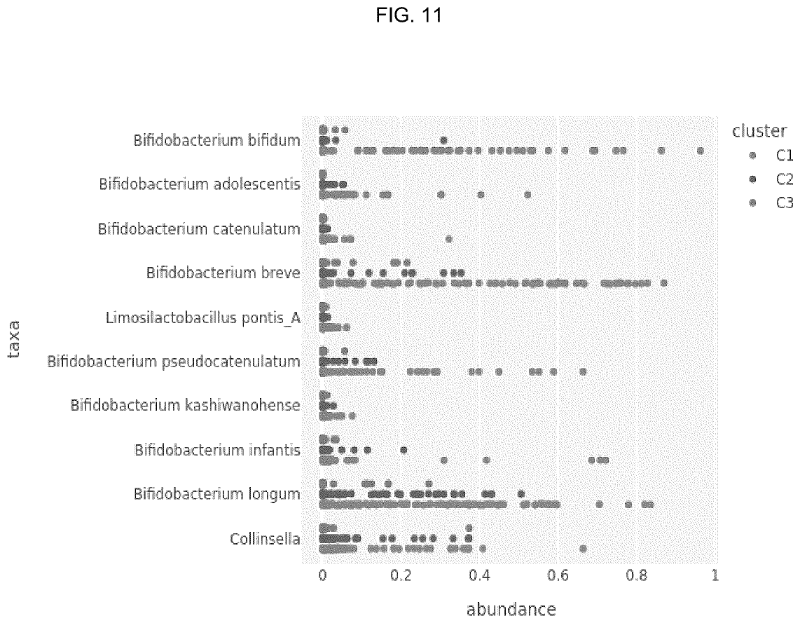

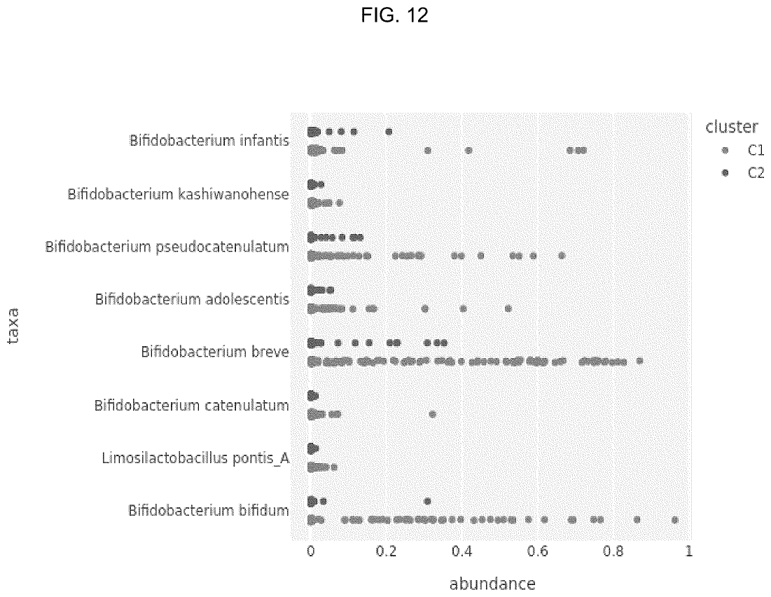

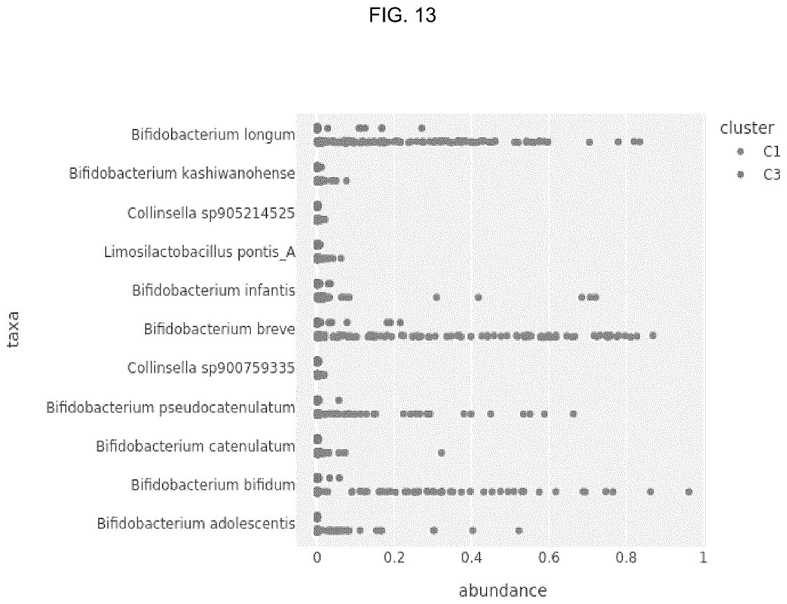

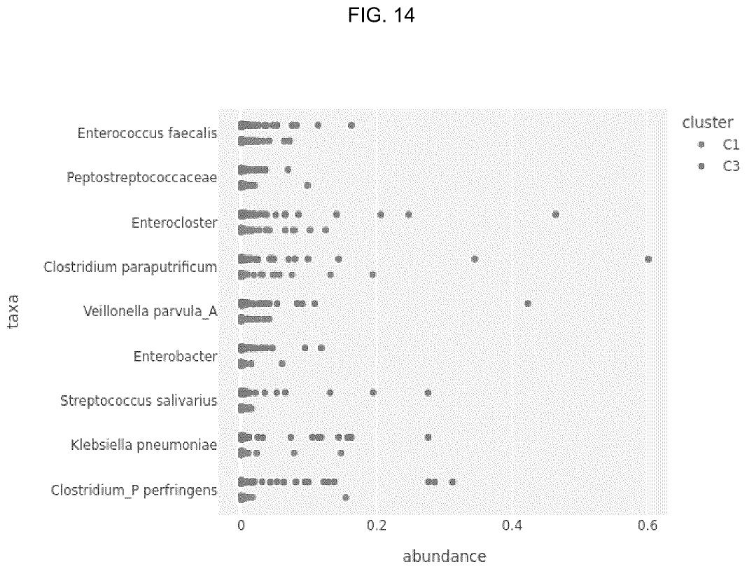

The fold change difference and statistical significance (inverse p value, Mann Whitney U test) was calculated for abundances of taxa in C1 relative to the other clusters, and the results displayed on a volcano plot (FIG. 10). Each point refers to a family, order, class, genus, or species. After eliminating taxa with low overall abundance, approximately 18 taxa are enriched in C1 with p values lower 1E-5. FIG. 11 to FIG. 13 and Table 1 show the abundances in each sample of the most significantly enriched species. The species enriched in C1 are Bifidobacterium infantis, Bifidobacterium bifidum, Bifidobacterium breve, Bifidobacterium longum, Bifidobacterium catenulatum, Bifidobacterium pseudocatenulatum, Bifidobacterium adolescentis, Collinsella sp900759335, and Limosilactobacillus pontis_A. In addition, 9 taxa are enriched in C3 relative to C1, many of which are or contain potentially pathogenic species (FIG. 14).

Table 1: List of taxa enriched or depleted between clusters or groups of clusters.

Similar results were obtained using the alternate KRAKEN2™ with custom index classification method. With the KRAKEN2™ classification results we applied additional filters beyond statistical significance; using bootstrapping we removed enrichments that were not robust, and we filtered out enrichments where abundances were low compared to our estimated classification noise level. The cluster enrichment summary generated from this method is in Table 9.

Table 9: Summary List of taxa enriched between clusters or groups of clusters using KRAKEN2™ with custom classification index and filtering on statistical significance, robustness, and abundance.

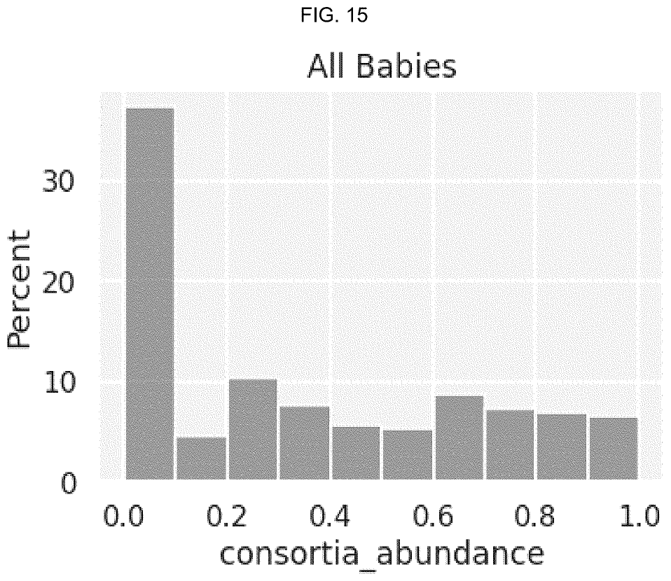

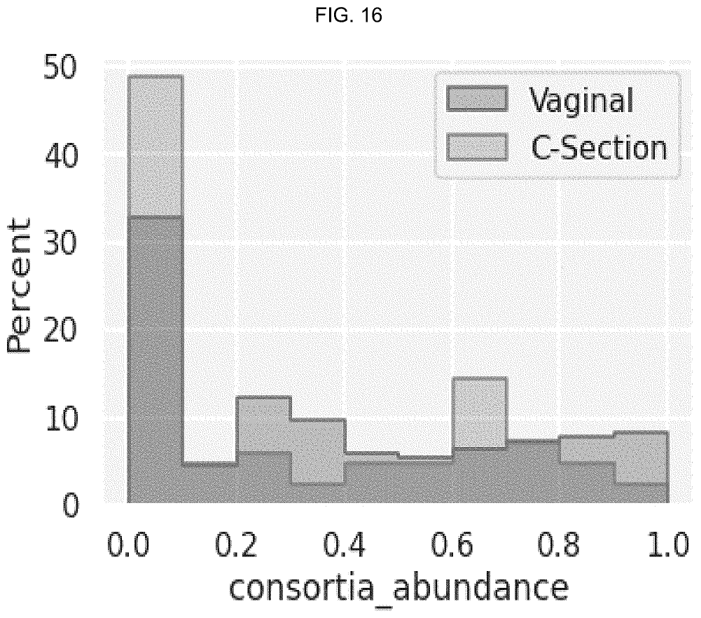

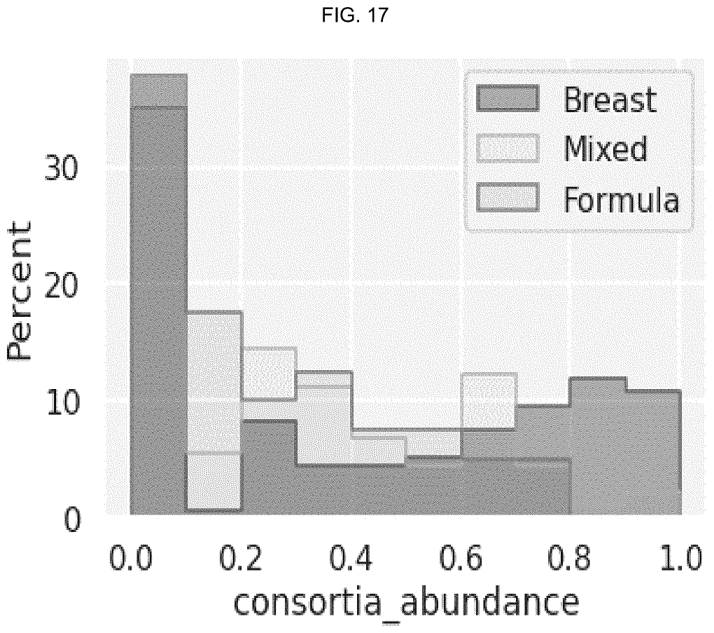

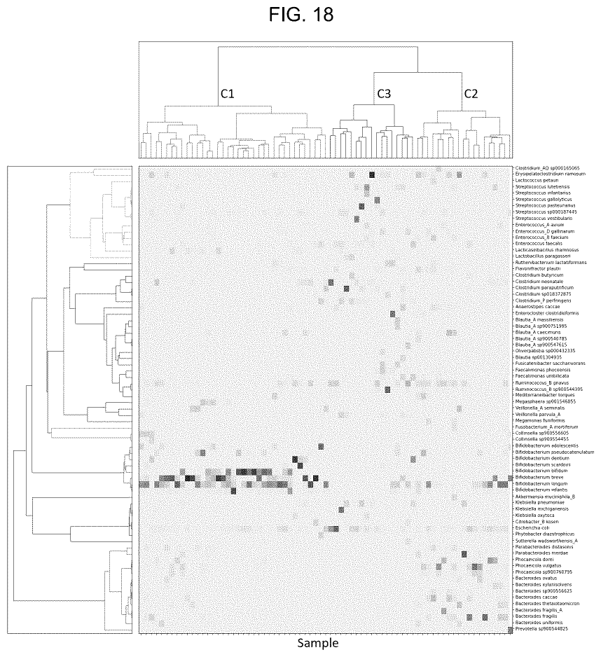

Four (4) of the Bifidobacterium species enriched in C1 are B. longum Sub. longum (B. longum), B. longum Sub. infantis (B. infantis), B. breve, and B. bifidum. These species are reported to be important to a healthy infant gut, in part because of their ability to consume human milk oligosaccharides (HMOs) (Underwood, M. A. et al. (2015) Pediatr. Res. 77:229-235; Sakanaka, M. et al. (2020) Nutrients 12:1-21). Despite their importance, relatively few of our samples contain these organisms at high levels, and in many they are absent altogether (FIG. 15). Total abundance of these core species is even lower for C-section born (FIG. 16) and formula fed (FIG. 17) infants. Abundance of Bifidobacterium in general, and these 4 species in particular, is high in many of the C1 samples (FIG. 18).

TABLE 1
List of taxa enriched or depleted between
clusters or groups of clusters.

| name | taxRank | category | in_cluster | vs_cluster |
|---|---|---|---|---|
| Enterococcus faecalis | species | enriched | C3 | C1 |
| Enterococcus | genus | enriched | C3 | C1 |
| Enterococcaceae | family | enriched | C3 | C1 |
| Streptococcus salivarius | species | enriched | C3 | C1 |
| Streptococcus | genus | enriched | C3 | C1 |
| Streptococcaceae | family | enriched | C3 | C1 |
| Lactobacillies | order | enriched | C3 | C1 |
| Bacilli | class | enriched | C3 | C1 |
| Firmicutes | phylum | enriched | C3 | C1 |
| Clostridium paraputrificum | species | enriched | C3 | C1 |
| Clostridium | genus | enriched | C3 | C1 |
| Clostridium_{—}P perfringens | species | enriched | C3 | C1 |
| Clostridium_{—}P | genus | enriched | C3 | C1 |
| Clostridiaceae | family | enriched | C3 | C1 |
| Clostridiales | order | enriched | C3 | C1 |
| Enterocloster | genus | enriched | C3 | C1 |
| Peptostreptococcales | order | enriched | C3 | C1 |
| Clostridia | class | enriched | C3 | C1 |
| Firmicutes_{—}A | phylum | enriched | C3 | C1 |
| Veillonella parvula_{—}A | species | enriched | C3 | C1 |
| Veillonella | genus | enriched | C3 | C1 |
| Veillonellaceae | family | enriched | C3 | C1 |
| Enterobacter | genus | enriched | C3 | C1 |
| Klebsiella pneumoniae | species | enriched | C3 | C1 |
| Klebsiella | genus | enriched | C3 | C1 |
| Enterobacteriaceae | family | enriched | C3 | C1 |
| Enterobacterales | order | enriched | C3 | C1 |
| Gammaproteobacteria | class | enriched | C3 | C1 |
| Proteobacteria | phylum | enriched | C3 | C1 |
| Bifidobacterium | species | enriched | C1 | C2 and C3 |
| adolescentis | | | | |
| Bifidobacterium bifidum | species | enriched | C1 | C2 and C3 |
| Bifidobacterium breve | species | enriched | C1 | C2 and C3 |
| Bifidobacterium | species | enriched | C1 | C2 and C3 |
| catenulatum | | | | |
| Bifidobacterium infantis | species | enriched | C1 | C2 and C3 |
| Bifidobacterium | species | enriched | C1 | C2 and C3 |
| kashiwanohense | | | | |
| Bifidobacterium longum | species | enriched | C1 | C2 and C3 |
| Bifidobacterium | species | enriched | C1 | C2 and C3 |
| pseudocatenulatum | | | | |
| Bifidobacterium | genus | enriched | C1 | C2 and C3 |
| Bifidobacteriaceae | family | enriched | C1 | C2 and C3 |
| Actinomycetales | order | enriched | C1 | C2 and C3 |
| Actinomycetia | class | enriched | C1 | C2 and C3 |
| Collinsella sp900759335 | species | enriched | C1 | C2 and C3 |
| Collinsella | genus | enriched | C1 | C2 and C3 |
| Coriobacteriaceae | family | enriched | C1 | C2 and C3 |
| Coriobacteriales | order | enriched | C1 | C2 and C3 |
| Coriobacteriia | class | enriched | C1 | C2 and C3 |
| Actinobacteriota | phylum | enriched | C1 | C2 and C3 |
| Limosilactobacillus | species | enriched | C1 | C2 and C3 |
| pontis_{—}A | | | | |
| Limosilactobacillus | genus | enriched | C1 | C2 and C3 |
| Lactobacilluseae | family | enriched | C1 | C2 and C3 |
| Bifidobacterium | species | depleted | C2 | C1 |
| adolescentis | | | | |
| Bifidobacterium bifidum | species | depleted | C2 | C1 |
| Bifidobacterium breve | species | depleted | C2 | C1 |
| Bifidobacterium | species | depleted | C2 | C1 |
| catenulatum | | | | |
| Bifidobacterium infantis | species | depleted | C2 | C1 |
| Bifidobacterium | species | depleted | C2 | C1 |
| kashiwanohense | | | | |
| Bifidobacterium | species | depleted | C2 | C1 |
| pseudocatenulatum | | | | |
| Bifidobacterium | genus | depleted | C2 | C1 |
| Bifidobacteriaceae | family | depleted | C2 | C1 |
| Actinomycetales | order | depleted | C2 | C1 |
| Actinomycetia | class | depleted | C2 | C1 |
| Actinobacteriota | phylum | depleted | C2 | C1 |
| Limosilactobacillus | species | depleted | C2 | C1 |
| pontis_{—}A | | | | |
| Limosilactobacillus | genus | depleted | C2 | C1 |
| Lactobacilluse | family | depleted | C2 | C1 |
| Lactobacillus | order | depleted | C2 | C1 |
| Bacteroides caccae | species | enriched | C2 | C1 |
| Bacteroides fragilis | species | enriched | C2 | C1 |
| Bacteroides ovatus | species | enriched | C2 | C1 |
| Bacteroides sp900755095 | species | enriched | C2 | C1 |
| Bacteroides sp900766005 | species | enriched | C2 | C1 |
| Bacteroides | species | enriched | C2 | C1 |
| thetaiotaomicron | | | | |
| Bacteroides uniformis | species | enriched | C2 | C1 |
| Bacteroides | genus | enriched | C2 | C1 |
| Phocaeicola dorei | species | enriched | C2 | C1 |
| Phocaeicola sp900760795 | species | enriched | C2 | C1 |
| Phocaeicola vulgatus | species | enriched | C2 | C1 |
| Phocaeicola | genus | enriched | C2 | C1 |
| Prevotella | genus | enriched | C2 | C1 |
| Bacteroidaceae | family | enriched | C2 | C1 |
| Odoribacter sp900762515 | species | enriched | C2 | C1 |
| Odoribacter | genus | enriched | C2 | C1 |
| Marinifilaceae | family | enriched | C2 | C1 |
| Parabacteroides distasonis | species | enriched | C2 | C1 |
| Parabacteroides | genus | enriched | C2 | C1 |
| Tannerellaceae | family | enriched | C2 | C1 |
| Bacteroidales | order | enriched | C2 | C1 |
| Bacteroidia | class | enriched | C2 | C1 |
| Bacteroidota | phylum | enriched | C2 | C1 |
| Klebsiella pneumoniae | species | enriched | C2 | C1 |
| Enterobacterales | order | enriched | C2 | C1 |
| Gammaproteobacteria | class | enriched | C2 | C1 |
| Proteobacteria | phylum | enriched | C2 | C1 |
| Bifidobacterium | species | depleted | C3 | C1 |
| adolescentis | | | | |
| Bifidobacterium bifidum | species | depleted | C3 | C1 |
| Bifidobacterium breve | species | depleted | C3 | C1 |
| Bifidobacterium | species | depleted | C3 | C1 |
| catenulatum | | | | |
| Bifidobacterium infantis | species | depleted | C3 | C1 |
| Bifidobacterium | species | depleted | C3 | C1 |
| kashiwanohense | | | | |
| Bifidobacterium longum | species | depleted | C3 | C1 |
| Bifidobacterium | species | depleted | C3 | C1 |
| pseudocatenulatum | | | | |
| Bifidobacterium | genus | depleted | C3 | C1 |
| Bifidobacteriaceae | family | depleted | C3 | C1 |
| Actinomycetales | order | depleted | C3 | C1 |
| Actinomycetia | class | depleted | C3 | C1 |
| Collinsella sp018382295 | species | depleted | C3 | C1 |
| Collinsella sp900759335 | species | depleted | C3 | C1 |
| Collinsella sp905214525 | species | depleted | C3 | C1 |
| Collinsella | genus | depleted | C3 | C1 |
| Coriobacteriaceae | family | depleted | C3 | C1 |
| Coriobacteriales | order | depleted | C3 | C1 |
| Coriobacteriia | class | depleted | C3 | C1 |
| Actinobacteriota | phylum | depleted | C3 | C1 |
| Limosilactobacillus | species | depleted | C3 | C1 |
| pontis_{—}A | | | | |
| Limosilactobacillus | genus | depleted | C3 | C1 |

TABLE 9

| name | taxRank | enriched | VS |
|---|---|---|---|
| Actinobacteriota | phylum | C1 | C2 and C3 |
| Actinomycetales | order | C1 | C2 and C3 |
| Actinomycetia | class | C1 | C2 and C3 |
| Bifidobacteriaceae | family | C1 | C2 and C3 |
| Bifidobacterium | genus | C1 | C2 and C3 |
| Bifidobacterium | species | C1 | C2 and C3 |
| adolescentis | | | |
| Bifidobacterium bifidum | species | C1 | C2 and C3 |
| Bifidobacterium breve | species | C1 | C2 and C3 |
| Bifidobacterium longum | species | C1 | C2 and C3 |
| Bifidobacterium | species | C1 | C2 and C3 |
| pseudocatenulatum | | | |
| Bacteroidaceae | family | C2 | C1 and C3 |
| Bacteroidales | order | C2 | C1 and C3 |
| Bacteroides | genus | C2 | C1 and C3 |
| Bacteroides caccae | species | C2 | C1 and C3 |
| Bacteroides fragilis | species | C2 | C1 and C3 |
| Bacteroides ovatus | species | C2 | C1 and C3 |
| Bacteroides | species | C2 | C1 and C3 |
| thetaiotaomicron | | | |
| Bacteroides uniformis | species | C2 | C1 and C3 |
| Bacteroidia | class | C2 | C1 and C3 |
| Bacteroidota | phylum | C2 | C1 and C3 |
| Parabacteroides | genus | C2 | C1 and C3 |
| Parabacteroides distasonis | species | C2 | C1 and C3 |
| Parabacteroides merdae | species | C2 | C1 and C3 |
| Phocaeicola | genus | C2 | C1 and C3 |
| Phocaeicola dorei | species | C2 | C1 and C3 |
| Phocaeicola vulgatus | species | C2 | C1 and C3 |
| Tannerellaceae | family | C2 | C1 and C3 |
| Bacilli | class | C3 | C1 and C2 |
| Clostridia | class | C3 | C1 and C2 |
| Clostridiaceae | family | C3 | C1 and C2 |
| Clostridiales | order | C3 | C1 and C2 |
| Clostridium | genus | C3 | C1 and C2 |
| Clostridium_{—}P | genus | C3 | C2 |
| Clostridium_{—}P perfringens | species | C3 | C2 |
| Enterobacterales | order | C3 | C1 and C2 |
| Enterobacteriaceae | family | C3 | C1 and C2 |
| Enterococcaceae | family | C3 | C2 |
| Enterococcus | genus | C3 | C2 |
| Enterococcus faecalis | species | C3 | C2 |
| Faecalimonas | genus | C3 | C1 |
| Firmicutes | phylum | C3 | C1 and C2 |
| Firmicutes_{—}A | phylum | C3 | C1 and C2 |
| Gammaproteobacteria | class | C3 | C1 and C2 |
| Klebsiella | genus | C3 | C1 and C2 |
| Klebsiella michiganensis | species | C3 | C1 |
| Klebsiella pneumoniae | species | C3 | C1 |
| Lachnospiraceae | family | C3 | C1 |
| Lachnospirales | order | C3 | C1 |
| Lactobacillies | order | C3 | C1 and C2 |
| Peptostreptococcaceae | family | C3 | C2 |
| Peptostreptococcaceae | family | C3 | C1 |
| Peptostreptococcales | order | C3 | C1 |
| Peptostreptococcales | order | C3 | C2 |
| Proteobacteria | phylum | C3 | C1 and C2 |
| Streptococcaceae | family | C3 | C1 and C2 |
| Streptococcus | genus | C3 | C1 and C2 |
| Streptococcus sp001556435 | species | C3 | C1 |
| Veillonella | genus | C3 | C2 |
| Veillonella parvula_{—}A | species | C3 | C1 and C2 |
| Veillonellaceae | family | C3 | C2 |

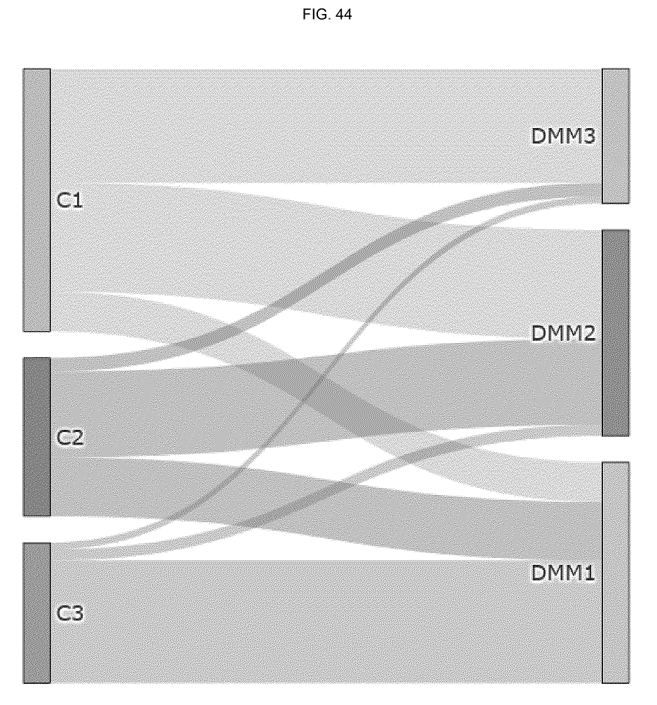

An alternative grouping of infant microbiomes was performed using a Dirichlet multinomial mixtures (DMM) clustering routine (Holms I. et al. (2012) PLOS One. 7 (2): e30126). The Dirichlet multinomial mixture analysis was performed with open source software (https://microbiome.github.io/tutorials/DMM.html) and returned 3 clusters with significant overlap compared to the GUNIFRAC™ (gUniFrac) clusters (chi-squared p-value <0.0001). The relationship between the GUNIFRAC™ clusters C1, C2, and C3; and the DMM clusters DMM1, DMM2, and DMM3 is illustrated in a sankey diagram (FIG. 44). We see that DMM3 is primarily composed of samples that are also members of C1, DMM1 is more closely associated with C3, and DMM2 is made up primarily of a combination of C1 and C2.

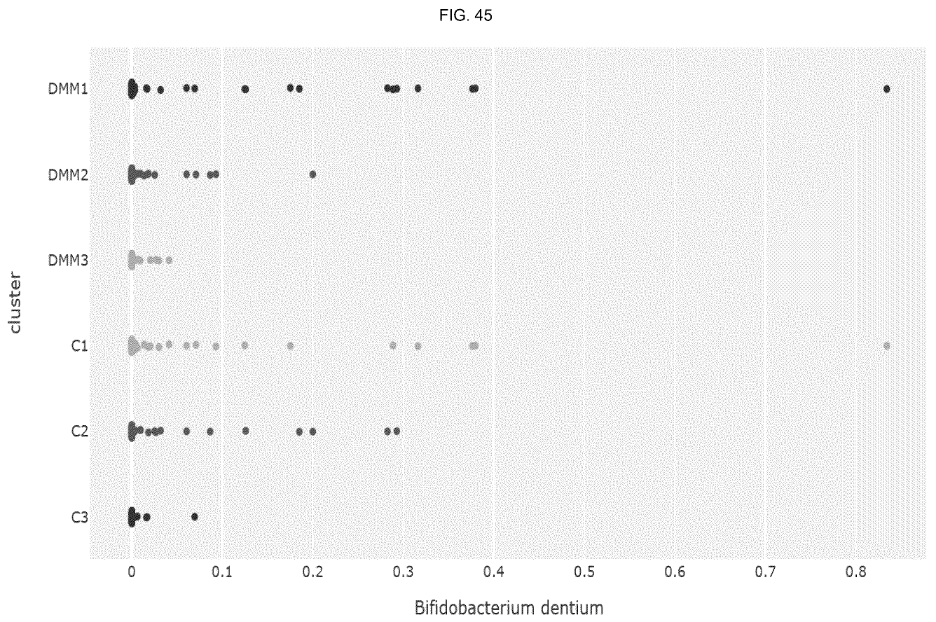

While the GUNIFRAC™ clusters are groups of samples with species close to each other on the taxonomic tree, Dirichlet multinomial mixtures group samples purely on joint taxa distributions regardless of evolutionary history. An example of the fundamental difference between GUNIFRAC™ clusters and DMM clusters is shown in FIG. 45 where we see Bifidobacterium dentium relative abundances both for the DMM clusters and the GUNIFRAC™ clusters (C1, C2, and C3). With GUNIFRAC™, samples with large relative abundances of B. dentium are grouped with samples having high relative abundances of the other Bifidobacteria; i.e. C1. B. dentium is not typically associated with a healthy infant gut, and with the DMM clusters we see that samples high in B. dentium are no longer in the healthy infant gut cluster (DMM3), but are located in a cluster that we consider a dysbiotic gut (DMM1).

The Dirichlet multinomial mixture models have statistically significant associations with both birth mode (vaginal vs cesarian) and feeding mode (breast, mixed, or formula); these associations are shown in Table 37 and Table 38.

TABLE 37
Observed number of infants vs what would be expected for
no association, for DMM clusters vs birth mode (chi-squared
p-value = 0.0076). DMM1 has an association with
C-Section, while DMM2 is associated with Vaginal birth;
e.g. 69 DMM1 infants were born via C-Section, but without
an association we would only expect 54.7.

| Observed:Expected | C-section | Vaginal |
|---|---|---|
| DMM1 | 69:54.7 | 93:107.3 |
| DMM2 | 40:50.9 | 111:100.1 |
| DMM3 | 30:33.4 | 69:65.6 |

TABLE 38
Observed number of infants vs what would be expected for
no association, for DMM clusters vs feeding mode (chi-
squared p-value = 0.0474). DMM3 has an association
with Breast Fed; e.g. DMM3 had 66 infants breast fed infants,
but without an association we would only expect 53.3.

| Observed:Expected | Breast | Mixed | Formula Fed |
|---|---|---|---|
| DMM1 | 85:87.3 | 55:54.3 | 22:20.4 |
| DMM2 | 71:81.4 | 59:50.6 | 21:19.1 |
| DMM3 | 66:53.3 | 24:33.2 | 9:12.5 |

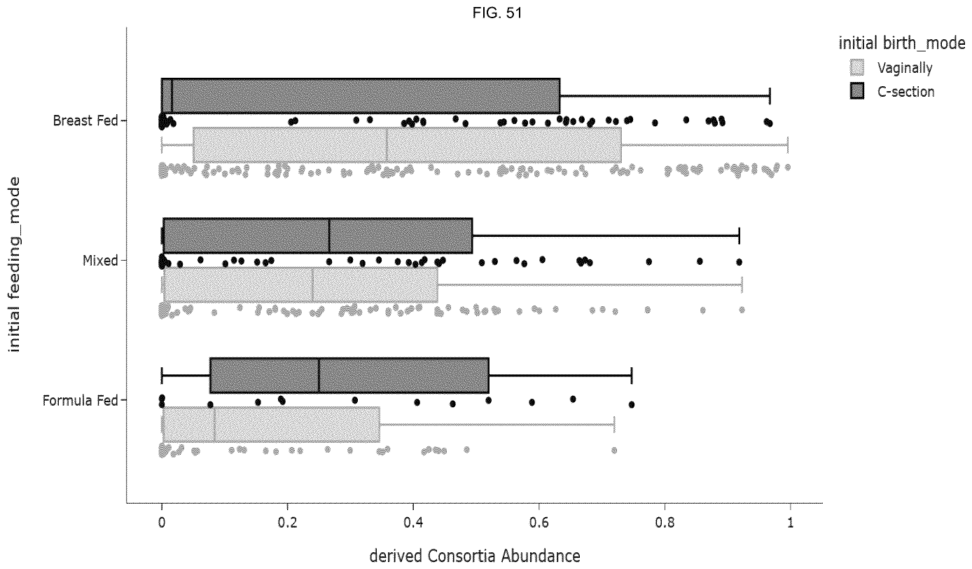

FIG. 51 shows Bifidobacterium consortia relative abundance (combination of B. infantis, B. breve, B. bifidum, and B. longum) separated by both feeding mode and birth mode. We see the median consortia abundance being highest for breast fed infants that are born vaginally, well outperforming the c-section infants also receiving a breast-milk diet. This is likely the result of the capacity of the consortia microbes to consume HMOs. Notably this trend is reversed for formula fed infants. Evidently other microbes typically transferred in vaginal birth are able to outperform the consortia microbes in metabolizing typical formula contents.

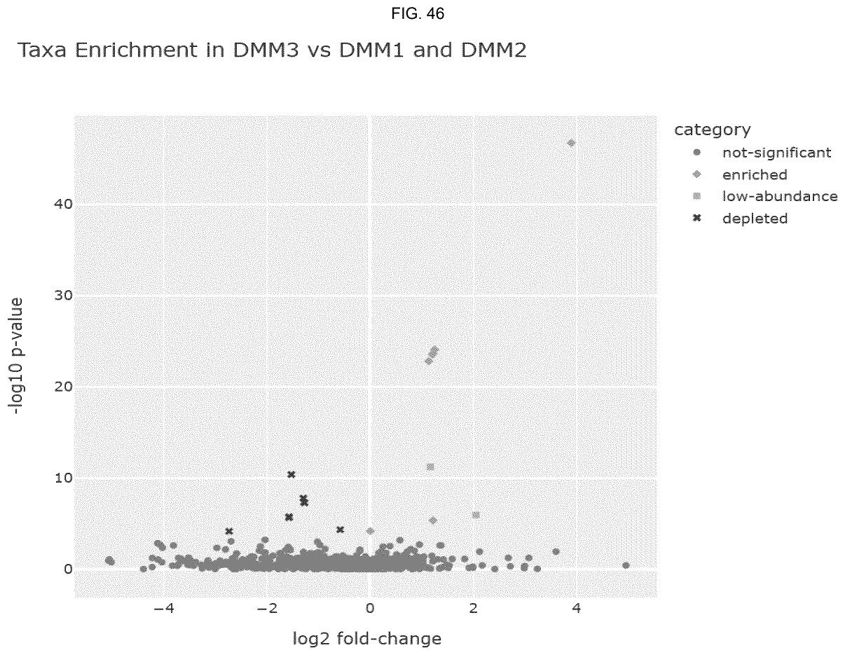

We looked at what species are enriched or depleted in the DMM clusters using the same method used for GUNIFRAC™ clusters described above. An example volcano plot showing taxa enriched in DMM3 vs DMM1 and DMM2 combined is shown in FIG. 46. The aggregated table of enriched taxa for the three DMM clusters is provided in Table 39.

TABLE 39
Key enriched taxa for the Dirichlet multinomial mixture
clusters. DMM3 is considered a healthy infant gut.

| name | taxRank | Cluster |
|---|---|---|
| Enterococcus faecalis | species | DMM1 |
| Streptococcus salivarius | species | DMM1 |
| Streptococcus sp001556435 | species | DMM1 |
| Clostridium | genus | DMM1 |
| Clostridium_{—}P perfringens | species | DMM1 |
| Lachnospiraceae | family | DMM1 |
| Veillonella parvula_{—}A | species | DMM1 |
| Klebsiella michiganensis | species | DMM1 |
| Klebsiella pneumoniae | species | DMM1 |
| Bifidobacterium longum | species | DMM2 |
| Collinsella | genus | DMM2 |
| Bacteroides fragilis | species | DMM2 |
| Bacteroides ovatus | species | DMM2 |
| Bacteroides | species | DMM2 |
| thetaiotaomicron | | |
| Phocaeicola dorei | species | DMM2 |
| Phocaeicola vulgatus | species | DMM2 |
| Bifidobacterium bifidum | species | DMM3 |
| Bifidobacterium breve | species | DMM3 |
| Bifidobacterium infantis* | species | DMM3 |

*Bifidobacterium infantis was enriched in DMM3 but didn't pass qc thresholds for abundance or robustness; we report it here because of the species known functionality in HMO catabolismand inclusion in other datasets within this document.

Antibiotic resistance markers in metagenomic samples were detected using the NCBI NATIONAL DATABASE OF ANTIBIOTIC RESISTANT ORGANISMS™ (NDARO). The genomes used to build the KRAKEN2™ database for classification (described above) were analyzed using prodigal (Hyatt, D. et. Al, BMC Bioinformatics 11, 119 (2010)) for open reading frame identification. Those open reading frames were then BLAST searched against the set of genes in the NCBI antimicrobial gene index to add a functional annotation to the gene set. The metagenomic sequencing data for each infant fecal sample was then searched against the annotated gene list using CENTRIFUGE™ (Kim et al. (2016) Genome Research 26:1721-1729). The number of antimicrobial resistance (AMR) signatures detected for each sample was tabulated.

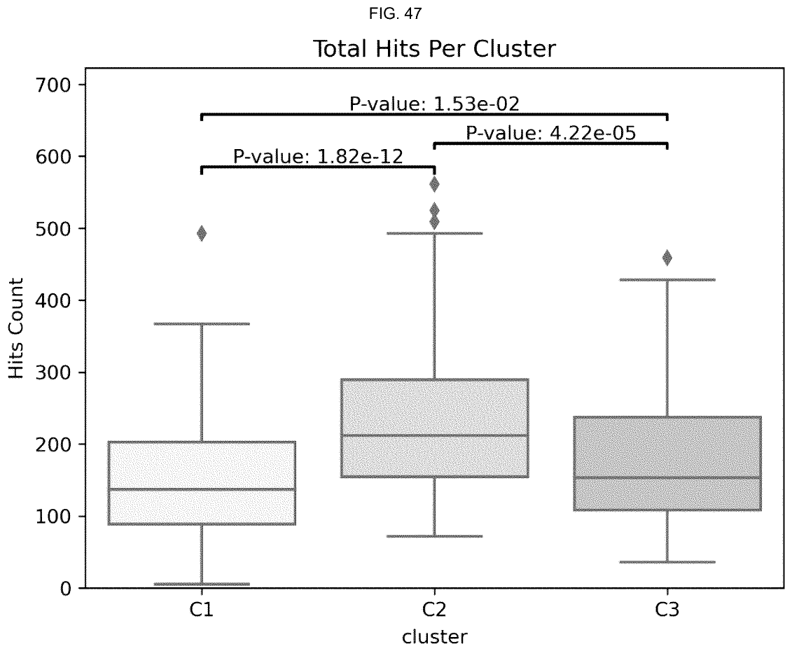

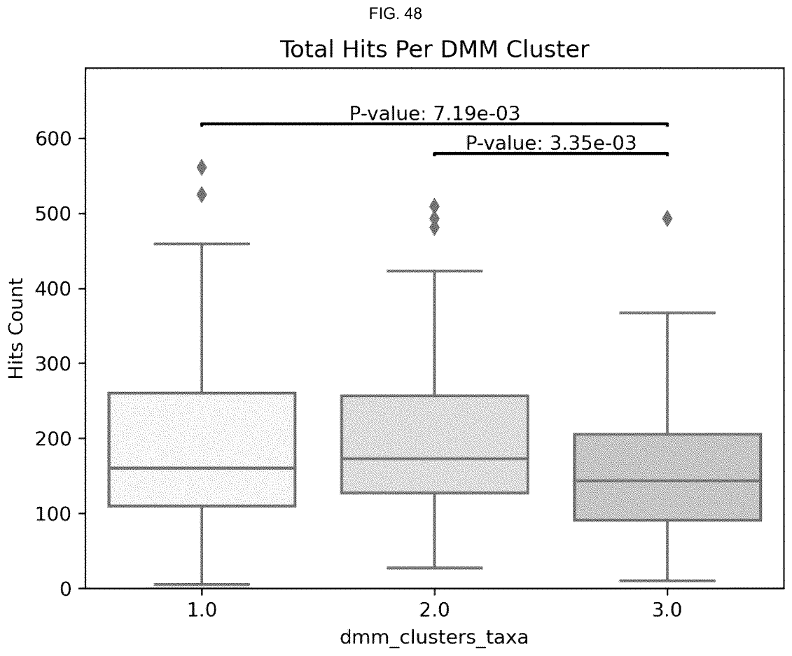

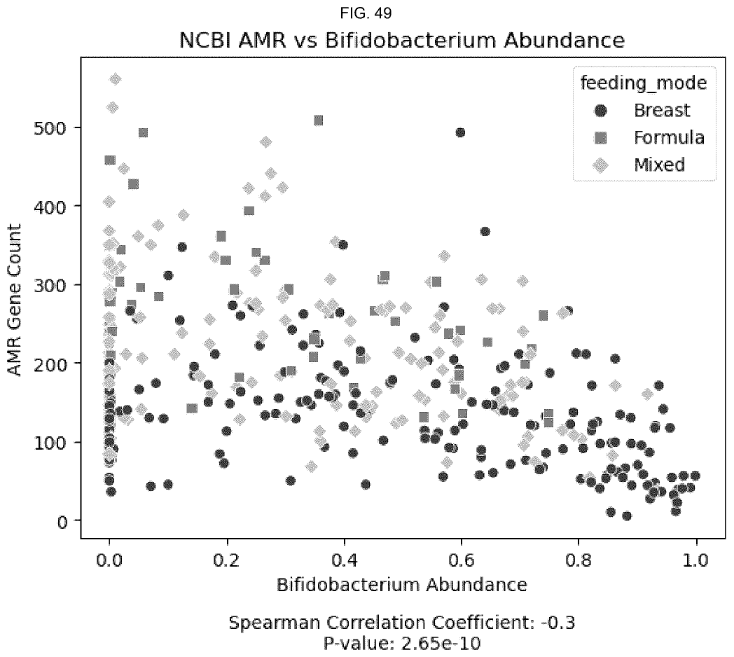

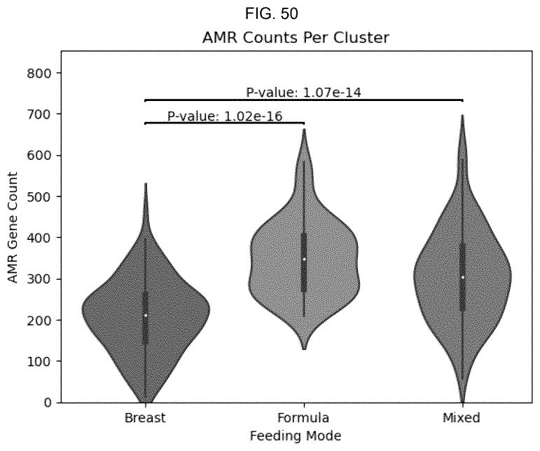

Box and whisker plots showing the distribution of number of AMR signatures for each sample separated by GUNIFRAC™ cluster are shown in FIG. 47 and for the DMM clusters in FIG. 48. We see that the clusters associated with healthy infant gut microbiomes C1 and DMM3 have a statistically significant reduced number of AMR signatures. FIG. 49 shows the inverse correlation between Bifidobacterium abundance and the number of AMR signatures, and also shows an observed trend between feeding mode and AMR signatures. FIG. 50 shows the statistically significant differences in the distributions of AMR signatures grouped by feeding mode, with Breast fed gut microbiomes having the lowest median AMR count, followed by Mixed, and finally Formula with the highest.

### MY BABY BIOME™ Clinical Study and Health Outcomes

To associate health outcomes with infant microbiomes features we both analyzed published data from the “3 country cohort” of the DIABIMMUNE™ study (Vatanen T. et al. (2016) Cell. 165:842-853) and obtained updated health information from the participants of the MY BABY BIOME™ Study.

### DIABIMMUNE™

The DIABIMMUNE™ “3 country cohort” data tracked children from birth to 3 years old, to better understand the prevalence of allergy and autoimmune disease in industrialized societies. Fecal samples were taken frequently for each participant, from birth to 3 years old, and health status for each participant is provided covering the first 3 years of life.

The gut microbiome sequencing data from DIABIMMUNE™ is 16s rRNA (compared to MY BABY BIOME™ whole genome sequencing) and thus is not able to resolve all species and strain level features. We analyze features of the data at the genus level. Specifically, we look at the ability of Bifidobacterium abundance to predict IGE levels and allergy incidence taken at two age cohorts: 104 samples taken when participants were between birth and 110-days of age (most similar to the MY BABY BIOME™ cohort), and 189 samples taken when participants were between 110-days to 1-year old. We selected one sample for each participant in each age group, choosing the sample closest in age to the centroid age for the cohort.

From birth to 110 days, Table 33 provides the fold change and p-values (Mann-Whitney U test) for Bifidobacterium abundance associated with 18 metadata features. Mean_fc is mean Bifidobacterium abundance for individuals having a true value for the metadata field divided by the mean Bifidobacterium abundance for not having that condition; i.e. mean_fc of 0.62 for regular formula means Bifidobacterium tends to be higher in individuals that have not been feeding on regular formula. The only trends (statistical significance under 0.1) observed in the birth to 110-day old cohort, are infants on regular formula or hydrosylated formula having lower Bifidobacterium abundance.

TABLE 33
Bifidobacterium associations for guts between birth
and 110 days and metadata features (health outcomes
at 3-years) in the DIABIMMUNE ™ “3 country cohort”.

| Metadata feature | true_mean | false_mean | mean_fc | p_value |
|---|---|---|---|---|
| Exclusive | 0.200183 | 0.184468 | 1.085192 | 0.224908 |
| breast feeding | | | | |
| Regular formula | 0.136517 | 0.220886 | 0.618043 | 0.061841 |
| Hydrosylated | 0.143702 | 0.207983 | 0.690930 | 0.079399 |
| formula | | | | |
| Partly | 0.199405 | 0.187551 | 1.063203 | 0.199578 |
| hydrosylated | | | | |
| formula | | | | |
| Any baby formula | 0.160673 | 0.239035 | 0.672175 | 0.171107 |
| Abx first year | 0.211159 | 0.174585 | 1.209494 | 0.986822 |
| After abx | 0.451682 | 0.179594 | 2.515020 | 0.320750 |
| seroconverted | 0.100391 | 0.194587 | 0.515916 | 0.436490 |
| Allergy milk | 0.149272 | 0.212997 | 0.700817 | 0.346052 |
| Allergy egg | 0.120351 | 0.209052 | 0.575698 | 0.153784 |
| Allergy peanut | 0.223368 | 0.188409 | 1.185553 | 0.993852 |
| Allergy dust mite | 0.108481 | 0.194270 | 0.558400 | 0.594957 |
| Total-IGE high | 0.174307 | 0.195169 | 0.893105 | 0.789843 |
| Allergy cat | 0.243595 | 0.184984 | 1.316843 | 0.310348 |
| Allergy dog | 0.221252 | 0.187483 | 1.180116 | 0.469139 |
| Allergy birch | 0.257315 | 0.182070 | 1.413278 | 0.588145 |
| Allergy timothy | 0.326987 | 0.184575 | 1.771565 | 0.138124 |

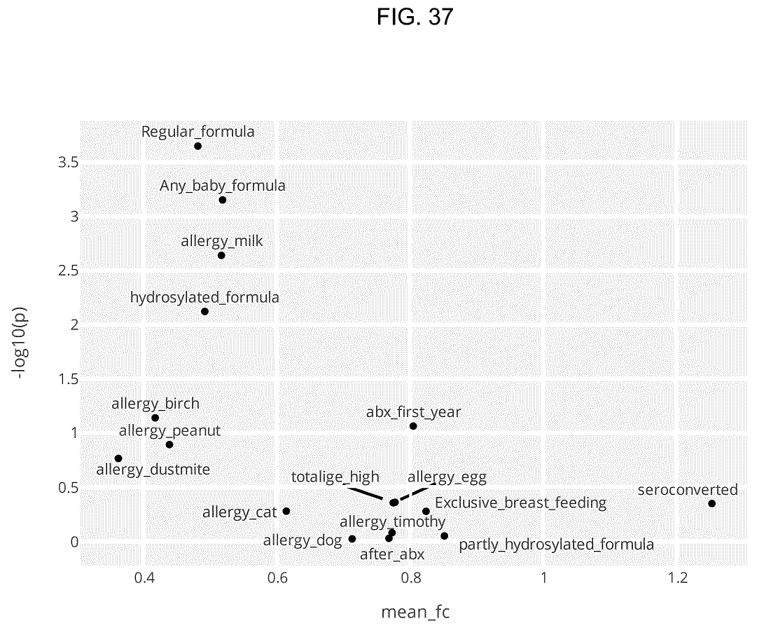

From 110 days to 1 year old we see more statistically significant associations with Bifidobacterium abundance. Table 34 lists the observed associations between Bifidobacterium abundance and metadata fields. Here we see statistically significant associations (p-value Mann-Whitney U under 0.01) between low Bifidobacterium abundance and: regular formula, hydrosylated formula, any baby formula, and milk allergy (by 3 years old). We also see trends (p-value Mann-Whitney U under 0.1) for antibiotics in the first year and birch allergy (by 3 years old), with Peanut allergy and dust mite allergy (by 3 years old) just under the threshold for significant trends. FIG. 37 graphs the fold change vs p-values for this cohort.

TABLE 34
Bifidobacterium associations for gut microbiomes between
110 days and 1 year old, and metadata features (health outcomes
at 3 years) in the DIABIMMUNE ™ 3 country cohort.

| name | true_mean | false_mean | mean_fc | p_value |
|---|---|---|---|---|
| Exclusive | 0.210437 | 0.255636 | 0.823188 | 0.535675 |
| breast feeding | | | | |
| Regular formula | 0.130370 | 0.271319 | 0.480505 | 0.000228 |
| Hydrosylated | 0.121913 | 0.248340 | 0.490913 | 0.007658 |
| formula | | | | |
| Partly | 0.193428 | 0.227360 | 0.850756 | 0.903985 |
| hydrosylated | | | | |
| formula | | | | |
| Any baby formula | 0.147793 | 0.285539 | 0.517593 | 0.000715 |
| Abx first year | 0.193620 | 0.240822 | 0.803997 | 0.087485 |
| After abx | 0.172665 | 0.224974 | 0.767489 | 0.950432 |
| seroconverted | 0.275042 | 0.219606 | 1.252432 | 0.453725 |
| Allergy milk | 0.133753 | 0.259307 | 0.515811 | 0.002325 |
| Allergy egg | 0.182125 | 0.235344 | 0.773867 | 0.447018 |
| Allergy peanut | 0.101344 | 0.231481 | 0.437809 | 0.129655 |
| Allergy dustmite | 0.083015 | 0.229860 | 0.361153 | 0.174186 |
| Total-ige high | 0.184100 | 0.237070 | 0.776565 | 0.441789 |
| Allergy cat | 0.141004 | 0.229904 | 0.613317 | 0.533932 |
| Allergy dog | 0.162819 | 0.228613 | 0.712203 | 0.962416 |
| Allergy birch | 0.097806 | 0.234894 | 0.416385 | 0.073397 |
| Allergy timothy | 0.175014 | 0.226669 | 0.772111 | 0.844285 |

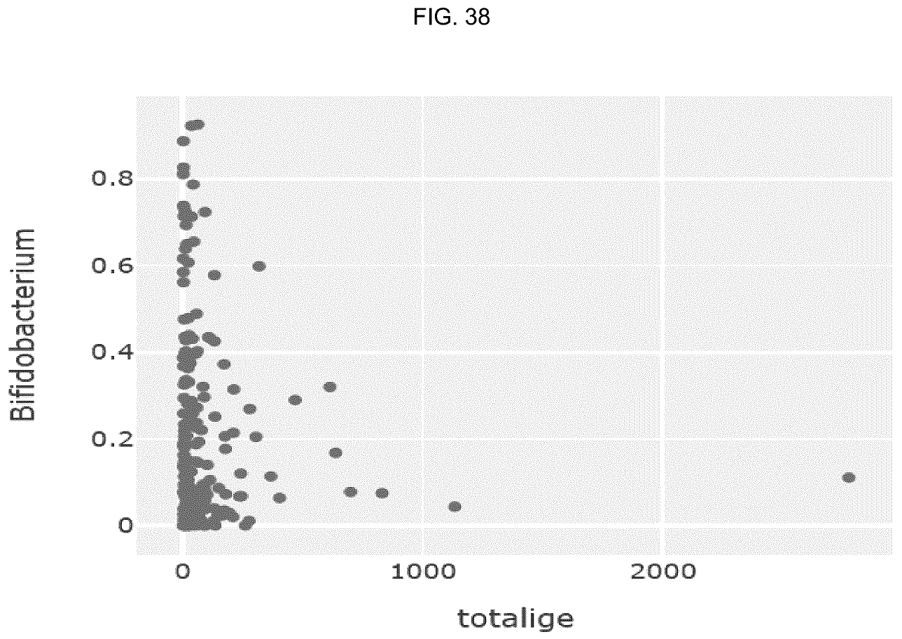

We also see a statistically significant trend between Bifidobacterium abundance between 110 days and 1 year and Total IGE (type-1 hypersensitivity marker) measurement by 3 years old (Spearman r=−0.185, p-value=0.013), meaning high Bifidobacterium is associated with low-IGE. FIG. 38 shows a scatter plot of Bifidobacterium abundance in the 110 day to 1 year range and 3 year-old total IGE measurement.

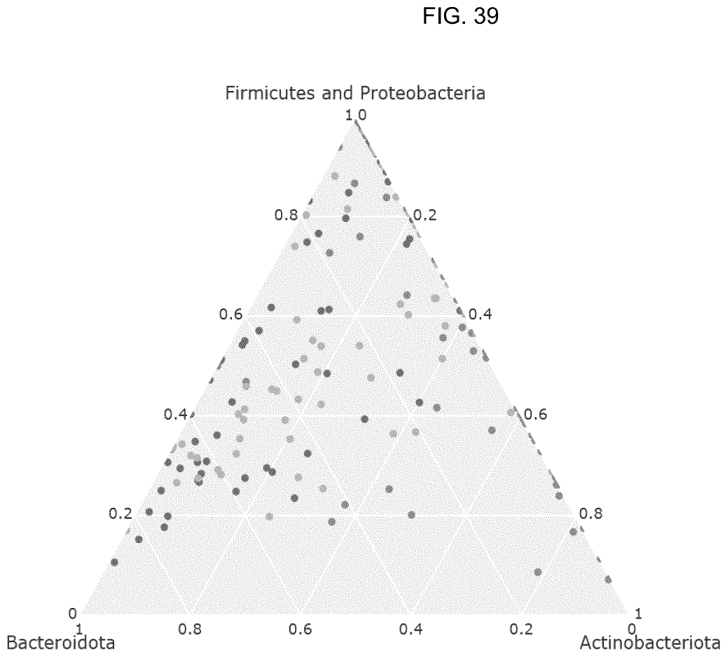

FIG. 39 shows that the gut microbiomes in the 110 day to 1 year range reflect the nation of origin, with Finland (representing and industrialized society) having the lowest Actinobacteria, Russia (Karelia, representing an agricultural society) having the most Actinobacteria but very little Bacteroidota, and Estonia (transitioning from agricultural into industrialized) having an intermediate amount of Actinobacteriota.

### MY BABY BIOME™ Clinical Study Follow Up Surveys

Participants in the MY BABY BIOME™ study were surveyed at 6 months and 1-year of age, with questions including health outcome information related to allergy and other immune-related complications. 26 participants responded with adverse health outcomes at 6-months (16 allergies, 2 Eczema, 9 Dermatitis, and 2 Asthma); and 46 at 1-year (35 allergies, 4 Eczema, 8 Dermatitis, and 3 asthma). These adverse outcomes were distributed throughout the sample population with little bias at 6-months for the cluster assignment of the original fecal sample. Statistical trends were observed between cluster and outcome at 1-year.

Trends associated with Bifidobacterium infantis, longum, breve, and bifidum abundances were searched for, as well as associated with the combination of all 4 species, but no statistically significant trends (Mann-Whitney U) were found for adverse outcomes combined at 6-months.

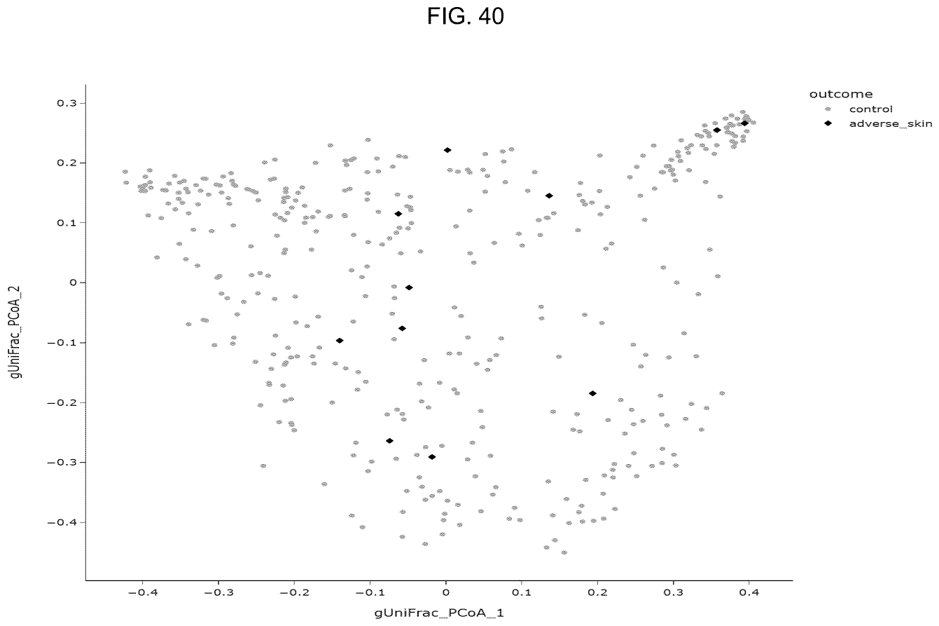

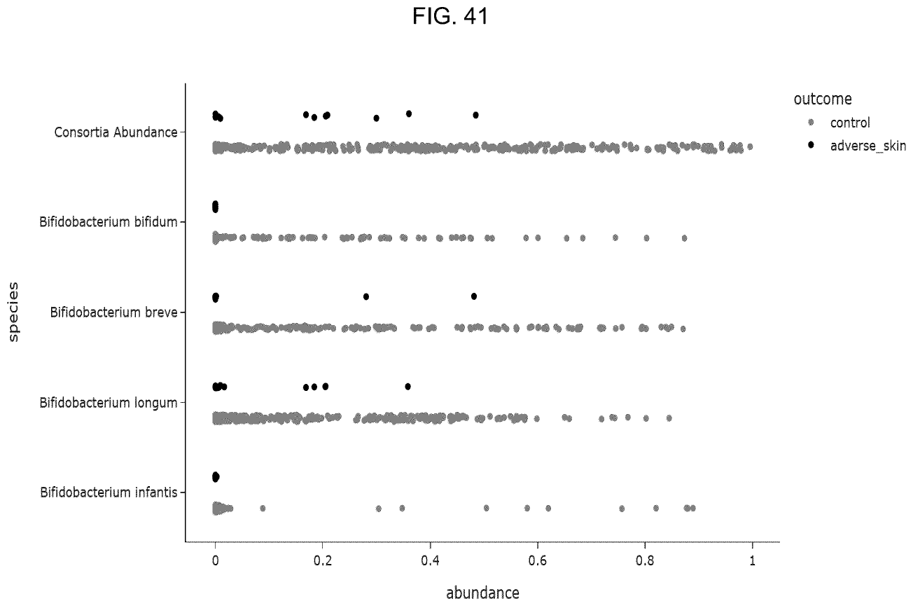

Exploring just the subset of dermatitis and eczema outcomes, we see that these skin conditions were not seen in the high Bifidobacterium region of the PCoA plot (FIG. 40). Table 35 lists the fold change and p-values (Mann-Whitney U) for Bifidobacterium infantis, longum, breve, and bifidum abundances as well as the combination of all 4 species. We see a large reduction of B. infantis and B. bifidum abundance between individuals with these skin conditions and those without them, though with limited data only B. bifidum has statistical significance. The abundances are plotted in FIG. 41.

TABLE 35
Fold change (mean of abundance with condition divided
by mean abundance without condition) and p-values
(Mann-Whitney U) for Eczema/Dermatitis.

| taxa | mean_fc | p_value |
|---|---|---|
| Bifidobacterium infantis | 0.029869 | 0.247407 |
| Bifidobacterium longum | 0.745504 | 0.960859 |
| Bifidobacterium breve | 0.608628 | 0.265140 |
| Bifidobacterium bifidum | 0.000000 | 0.099762 |
| Consortia Abundance | 0.549868 | 0.261732 |

In the 6-month survey data we also see a significant association between eczema and the C3 gUniFrac cluster (chi-squared p-val=0.049). Both participants with eczema at 6 months were in the C3 cluster. Both of those samples are also in DMM1, which has the highest overlap with C3, but because there are more samples in DMM1 than C3, the statistical significance is lower.

In the 1-year survey data we see a trend with DMM3 having fewer participants reporting eczema or dermatitis than expected for no relationship (chi-squared p-value=0.10). Table 36 shows the observed number of participants with either eczema or dermatitis at 1-year. We saw very similar trends at 6-months but with less statistical significance at that time-point (chi-squared p-value-0.2951 at 6-months vs 0.10 at 1-year).

TABLE 36
Observed number of infant samples vs what would be expected
for no association, for DMM clusters vs Eczema and Dermatitis
by 1-year. For example, with DMM3 we expected to see 2.7 infants
with eczema or dermatitis in the 1-year survey if there were
no association, but we saw 0. The Chi-squared p-value for the
association was p-value = 0.10 “a trend”. A similar
skew was present in the responses to the 6-month survey, but
the p-value was above the threshold for a reportable trend.

Eczema or Dermatitis
Observed:Expected

| | Without | With |
|---|---|---|
| DMM1 | 90:90.1 | 5:4.9 |
| DMM2 | 77:79.6 | 7:4.4 |
| DMM3 | 52:49.3 | 0:2.7 |

### Gene Function Analysis on Whole Genome Sequencing of Infant Fecal Samples

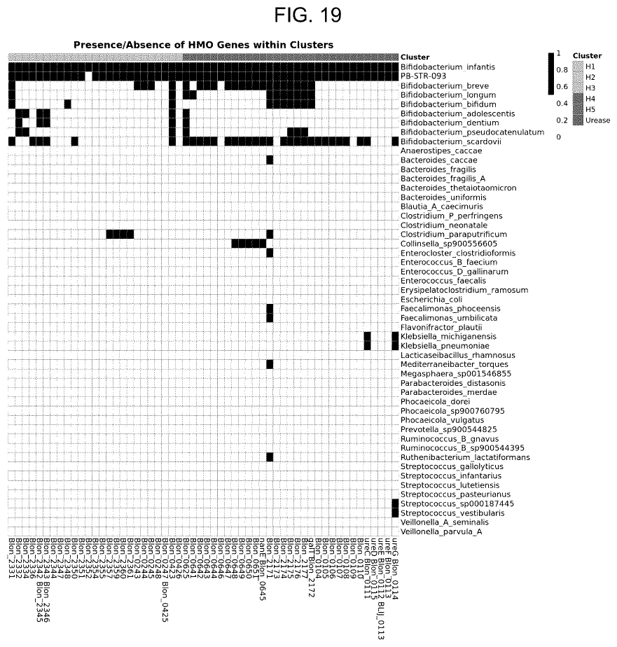

Published genomes as well as novel isolates were mined for the presence of known genes involved in HMO utilization (FIG. 19). These genes tend to group in 5 clusters, each responsible for metabolizing a different class of HMO, and a urease cluster (Sakanaka, M. et al. (2020) Nutrients 12:1-21). Only B. infantis isolates contain genes of all 5 HMO clusters plus the urease cluster, indicating that they are the most versatile at HMO utilization. B. breve contains most of the genes in clusters H2, H4, and H5, while B. longum and B. bifidum contain cluster H5 only, and B. scardovii has most genes in the H4, H5, and urease clusters. Several other genomes contain various HMO utilization genes, but none have a complete or nearly complete cluster.

Next, metagenomic sequences from each sample were screened for known HMO utilization genes. DIAMOND was used to map raw sequencing reads to a set of 56 genes belonging to 6 clusters: H1 (18 genes), H2 (4 genes), H3 (3 genes), H4 (12 genes), H5 (7 genes) and Urease (12 genes). Similarly, the abundances of other gene functions of interest in the samples are determined. These include genes encoding for production of acetate, lactate, butyrate, valerate, indole-3-lactate, indole-3-propionate, phenyllactate, phenylacetate, and bacteriocin.

### Metabolomics Analysis of Infant Samples

Fecal PBS samples isolated from infants study are evaluated by Liquid chromatography-mass spectrometry (LC-MS)/MS using a Sciex Exion UHPLC (Ultra-High-Performance Liquid Chromatography) coupled to a SCIEX 5500+TRIPLE QUADRUPOLE MASS SPECTROMETER™. A panel of 79 metabolites is evaluated (2-methylbutyrate, 3-hydroxybenzoate, 3-hydroxyhippurate, 3-hydroxyphenylpropionate, 3-methylindole, 4-ethylphenol, 4-Ethylphenylsulfate, 4-hydroxyphenylacetate, 4-hydroxyphenylacrylate, 4-hydroxyphenyllactate, 4-hydroxyphenylpropionate, acetate, agmatine, arginine, benzoate, betaine, butyrate, cadaverine, carnitine, chenodeoxycholate, cholate, choline, cinnamoylglycine, citulline, deoxycholate, enterodiol, enterolactone, glycochenodeoxycholate, glycocholate, hexanoate, Hippurate, imidazole propionate, indole, indole-3-acetamide, indole-3-lactate, indole-3-propionate, indoleacetate, indoleacetylglycine, indoleacrylate, indoleacrylglycine, indoxyl sulfate, inosine, isobutyrate, isoleucine, isovalerate, kynurenate, kynurenine, lactate, leucine, lithocholate, lysine, N-acetylserotonin, ornithine, p-cresol, p-cresol glucuronide, p-cresol sulfate, phenol, phenol glucuronide, phenol sulfate, phenylacetate, phenylacetylglutamine, phenylacetylglycine, phenylalanine, phenyllactate, phenylpropionate, phenylpropionylglycine, phenylpyruvate, propionate, putrescine, serotonin, thiamine, trimethylamine, tryptamine, tryptophan, tyramine, tyrosine, ursodeoxycholate, valerate, valine) through four different methods: reverse phase HPLC in positive mode, reverse phase HPLC in negative mode, HILIC in positive mode, and HILIC in negative mode. Absolute quantification for each sample is provided through a calibration curve and isotopically labeled internal standards. Values are normalized to fecal dry weight.

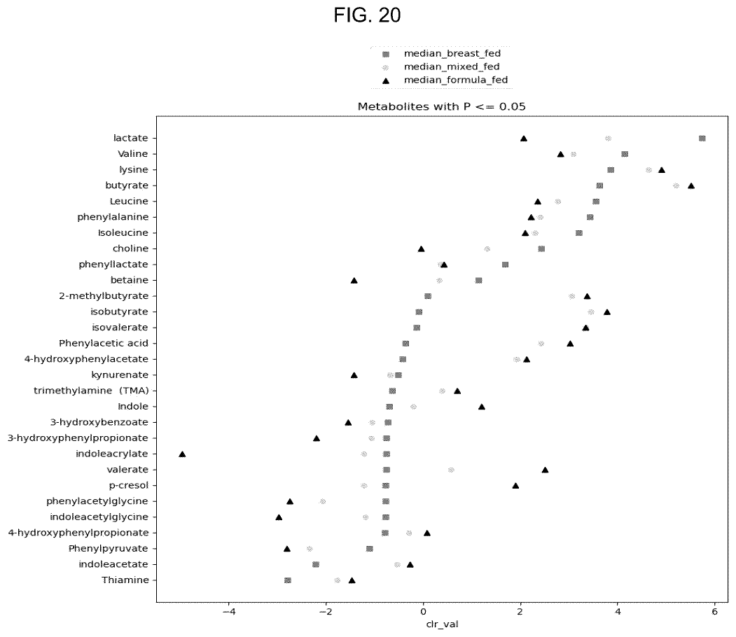

Samples were analyzed in the context of birth and feeding mode, and results revealed feeding mode as a significant driver of metabolism (FIG. 20). To eliminate the complication of feeding mode when interpreting metabolomics results, additional samples are evaluated to establish the unique metabolomes of our different clusters in the context of breast feeding.

### Protein and Cytokine Analysis of Infant Fecal Samples

Fecal PBS samples isolated from the infant study are evaluated for the presence of cytokines in the feces. This can be performed many ways, for example through use of a MESO QUICKPLEX SQ 120MM™ (Meso Scale Discovery) and MSD U-plex assay or through the use of LUMINEX™-based MILLIPLEX™ technology (Millipore). Panels of varying sizes are used depending on the application, for example a panel with 71 different cytokines (6CKine, BCA-1, CTACK, EGF, ENA-78, Eotaxin, Eotaxin-2, Eotaxin-3, FGF-2, Flt3L, Fractalkine, G-CSF, GM-CSF, GROα, I-309, IFNα2, IFNγ, IL-1α, IL-1β, IL-IRA, IL-2, IL-3, IL-4, IL-5, IL-6, IL-7, IL-8, IL-9, IL-10, IL-12p40, IL-12p70, IL-13, IL-15, IL-16, IL-17A, IL-17E/IL-25, IL-17F, IL-18, IL-20, IL-21, IL-22, IL-23, IL-27, IL-28, IL-33, IP-10, LIF, MCP-1, MCP-2, MCP-3, MCP-4, M-CSF, MDC, MIG, MIP-1a, MIP-1B, MIP-18, PDGF-AA, PDGF-AB/BB, RANTES, sCD40L, SCF, SDF-1α+β, TARC, TGFα, TNFα, TNFβ, TPO, TRAIL, TSLP, VEGF-A) provides insight into a broad range of cytokine expression in the gut. In addition to cytokine analysis, other proteins, for example calprotectin, are evaluated as potential markers of inflammation.

For the MY BABY BIOME™ study, 44 infant samples were analyzed using MILLIPLEX™ technology to evaluate a panel of 71 cytokines as mentioned above.

### Network Analysis of Multi-Omics Data

In order to identify relationships between high-dimensional microbiome, immune and metabolomics data, we started by reducing the feature set using Pearson correlation, then we estimated the inverse covariance using graphical lasso and constructed a network representation of the variables using NETWORKX™ (Aric A. Hagberg, et al, “Exploring network structure, dynamics, and function using NETWORKX™”, in Proceedings of the 7th Python in Science Conference (SciPy2008), Gäel Varoquaux, et al (Eds), (Pasadena, CA USA), pp. 11-15, August 2008). Starting with 50 metabolomics, shotgun metagenomics and immune samples, datasets were independently transformed using the centered log-ratio (CLR) prior to merging the three data types together. Pearson correlation was used to measure the linear association between pairs of variables in a dataset. P values were adjusted for multiple comparisons (Benjamini & Hochberg FDR) and only features with an adjusted p value <=0.05 were kept. The graphical lasso method was used to estimate the inverse covariance matrix of the variables in the reduced feature set. The inverse covariance matrix provides information about the conditional independence relationships between the variables and can be used to build a network representation. NETWORKX™, a Python library (Los Alamos National Laboratory) for analyzing graphs and networks, was used to construct a network representation of the variables based on the inverse covariance matrix estimated by the lasso method. The network representation visualized the relationships between variables and identified network modules, or clusters of variables that are highly interconnected. The Louvain community detection algorithm was applied in NETWORKX™ to identify network modules. These modules represent groups of variables that are highly interconnected and are likely to have similar biological functions or relationships. The modules were analyzed to identify key variables and understand the structure of the network.

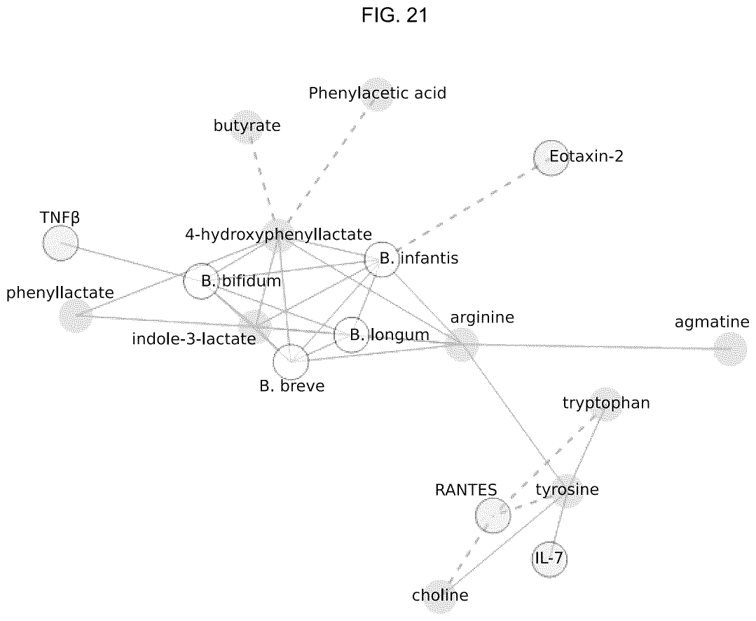

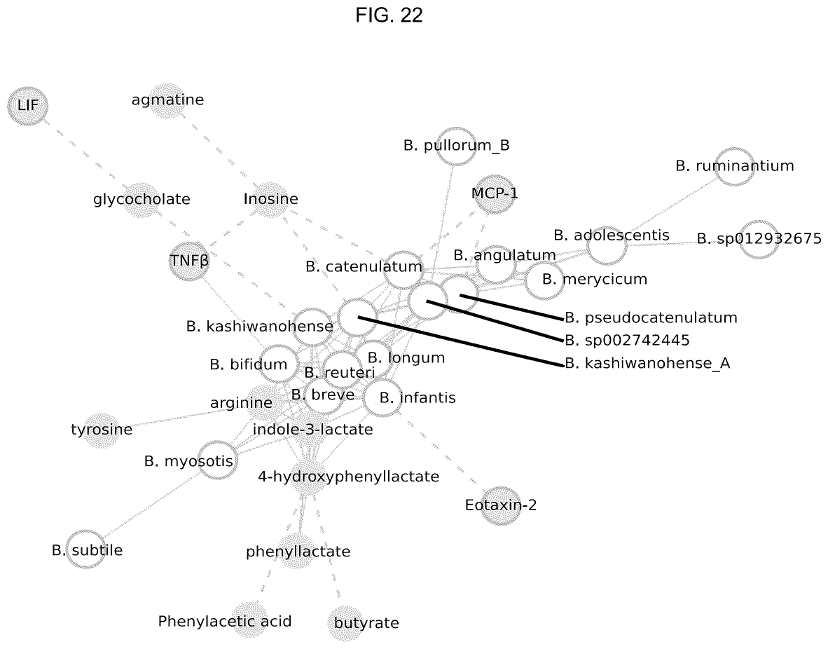

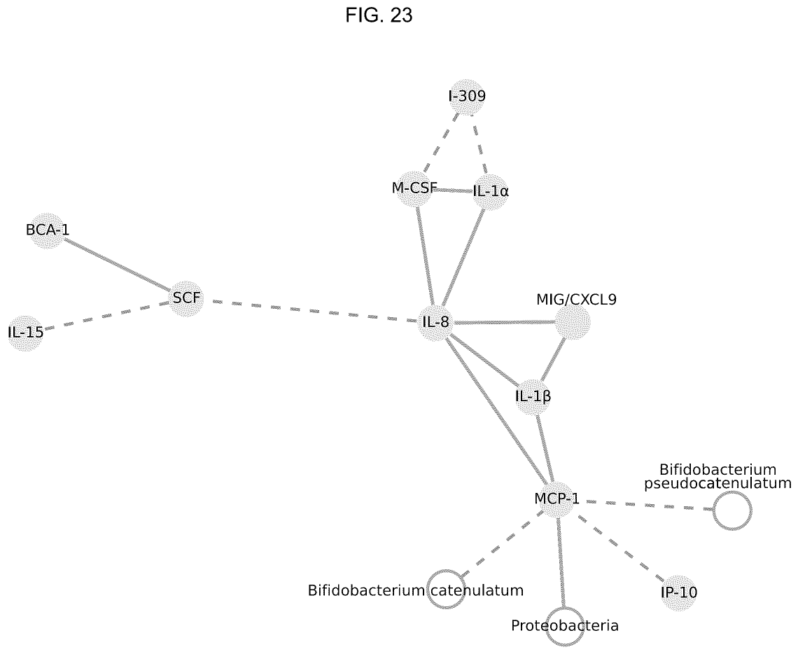

Using the above approaches, we saw that a notable core Bifidobacterium consortium (B. infantis, B. breve, B bifidum and B. longum) cluster tightly together and with a mostly anti-inflammatory response (immune and metabolite) (FIG. 21). When we added all Bifidobacterium species present in our infant metagenomes, there were a subset of Bifidobacterium that clustered closely together with the core Bifidobacterium consortia and anti-inflammatory response suggesting that they too possess anti-inflammatory behavior (FIG. 22). We next explored a network module containing the phylum Proteobacteria, since it has been shown to be associated with inflammation and is positively correlated with preterm infants. Our network analysis showed the Proteobacteria was indeed significantly associated with the proinflammatory chemokine MCP-1 (FIG. 23).

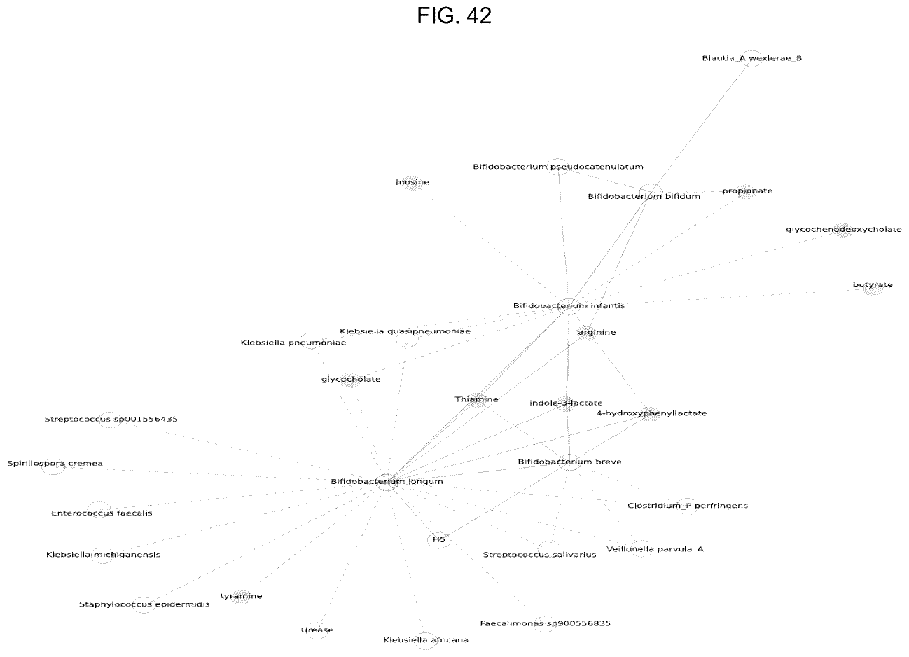

Network analysis was repeated using the same approach but applied to species composition of the samples obtained from the new KRAKEN2™ based classifier described in Example 3 (FIG. 42). Here we see three Bifidobacterium species (B. infantis, B. breve, and B. longum) with a positive association with the aromatic lactic acid derivatives indole-3-lactate and 4-phenyllactate. These molecules are thought to be critical to healthy immune system development in infants (Laursen M. et al. (2021) Nature Microbiology 6:1367-1382). B. longum is negatively associated with potentially harmful bacterial species, such as Klebsiella michiganensis, a known nosocomial pathogen (Simoni S. et al (2022) Antimicrobial Chemotherapy), and with metabolites such as trimethylamine, which has been associated with various chronic health conditions (Jalandra R. et al. (2023) Frontiers in Immunology 13). In contrast to the initial results described above, here B. bifidum clusters somewhat apart of the other 3 species. Because the KRAKEN2™ classifier reduces multimapping and issues with false positives, this new analysis provides additional insight into species level network connections.

### Metatranscriptomics Analysis

Fecal PBS samples are isolated from infants and preserved in an RNA preservation buffer at the time of isolation (such as ZYMO DNA/RNA SHIELD™ (ZYMO RESEARCH™)). RNA is extracted from the preservation buffer using a kit such as ZYMOBIOMICS™ MAGBEAD RNA (Zymo Research). RNA is evaluated for quality and quantity using a fluorometric technique, such as the QUBIT RNA HIGH SENSITIVITY ASSAY KIT™ (Invitrogen). Mammalian RNA is next recovered from the sample using a kit employing polyT hybridization, such as DYNABEADS™ mRNA DIRECT™ Purification Kit. This RNA is processed through reverse transcription and amplification using a kit such as the TRUSEQ STRANDED MRNA KIT™ (Illumina), and then prepared and analyzed using the same pipeline described for whole genome sequencing of DNA samples. The remaining RNA is processed using a kit such as the RIBO-ZERO PLUS MICROBIOME RRNA DEPLETION KIT™ (Illumina) and sequenced and analyzed using the same pipeline described for whole genome sequencing of DNA samples.

The genomes of gut microbes identified in the samples through metagenomics are used as a framework for analysis of the meta-transcriptomics analysis. Comparison of gene enrichment to transcript enrichment provides further insight into what is active in the gut environment.

### Example 4: Data Driven Approaches for Live Biotherapeutic Design

Based on the analysis performed in Example 3, four core strains of Bifidobacterium were selected as keystone species for biotherapeutic design. Bifidobacterium infantis, Bifidobacterium longum, Bifidobacterium breve, and Bifidobacterium bifidum were all shown to be statistically important species throughout the analyses performed in Example 3. Additional species were also shown to be statistically important (such as Bifidobacterium pseudocatenulatum), but based on a gUnifrac cladistic analysis of the C1 samples (as outlined in FIG. 18, Example 3), they did not cluster as clearly with the core four Bifidobacterium. All four core Bifidobacterium have known HMO consumption capabilities further demonstrating their relevance in the infant gut. Using the four keystone species, we developed a subset of biotherapeutic combinations that we then went on to further decorate with additional microbes (see for example Table 2 or Table 30). Most of these microbes were enriched in our own analyses (Table 1) or isolated as part of our bacterial isolation program (Example 5), demonstrating their relevance in a gut dominated by Bifidobacterium. A subset was identified as having complementary metabolism and therefore included as well.

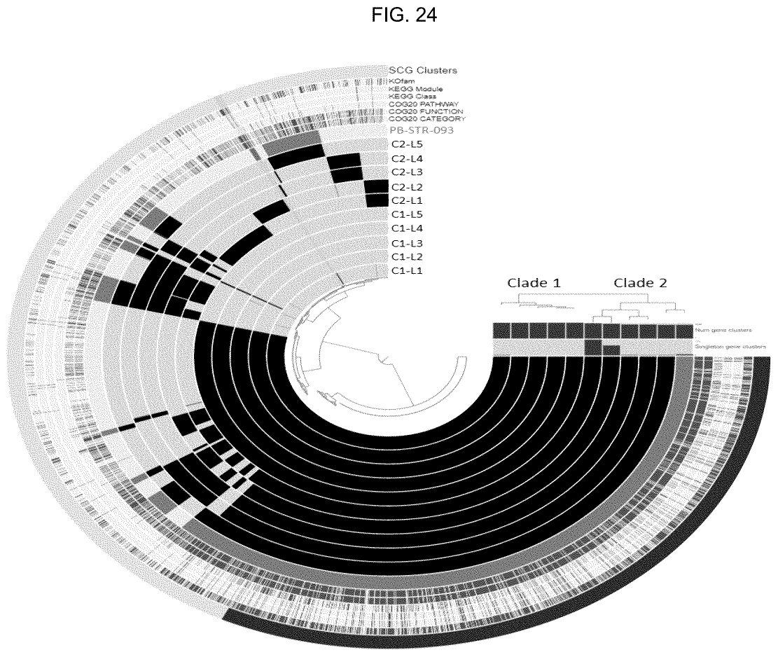

In some combinations, B. bifidum was excluded because it did not associate with the other three core species in the network analysis of FIG. 24. Furthermore, B bifidum carries genes that can produce 12,13-dihydroxy-9Z-octadecenoic acid (12,13—DiHOME) from linoleic acid. Although beneficial in small quantities, elevated fecal concentrations of this metabolite in infants have been associated with atopy and asthma in later childhood (Levan, S. R. et al., Nat. Microbiol. 2019, 4 (11): 1851-1861).

To complement the bacteria identified as a live biotherapeutic core, a set of prebiotics was selected. This set of probiotics was selected based on functions (i.e., HMO utilization) observed during the analysis performed in Example 3 and on in vitro growth evaluation. Due to the selectivity of HMOs for supporting Bifidobacterium growth they were chosen as the core of different prebiotic combinations (Table 3). These prebiotic cores were then supplemented with prebiotics (becoming synbiotics) expected to have synergistic growth effects or shown to have growth benefits through in vitro analyses.

In alternative embodiments, provided are combinations of bacteria (or probiotics) and prebiotics as set forth in Table 8, below.

TABLE 2
List of exemplary live biotherapeutic combinations,
mixes or consortia, or probiotics as provided herein:

| Combination | |
|---|---|
| Number | Included Bacteria |
| 1 | Bifidobacterium infantis |
| 2 | Bifidobacterium infantis |
| | Bifidobacterium breve |
| 3 | Bifidobacterium infantis |
| | Bifidobacterium breve |
| | Bifidobacterium longum |
| 4 | Bifidobacterium infantis |
| | Bifidobacterium breve |
| | Bifidobacterium bifidum |
| 5 | Bifidobacterium infantis |
| | Bifidobacterium bifidum |
| 6 | Bifidobacterium infantis |
| | Bifidobacterium bifidum |
| | Bifidobacterium longum |
| 7 | Bifidobacterium infantis |
| | Bifidobacterium longum |
| 8 | Bifidobacterium infantis |
| | Bifidobacterium kashiwanohense |
| 9 | Bifidobacterium infantis |
| | Bifidobacterium breve |
| | Bifidobacterium kashiwanohense |
| 10 | Bifidobacterium infantis |
| | Bifidobacterium breve |
| | Bifidobacterium longum |
| | Bifidobacterium kashiwanohense |
| 11 | Bifidobacterium infantis |
| | Bifidobacterium breve |
| | Bifidobacterium bifidum |
| | Bifidobacterium kashiwanohense |
| 12 | Bifidobacterium infantis |
| | Bifidobacterium bifidum |
| | Bifidobacterium kashiwanohense |
| 13 | Bifidobacterium infantis |
| | Bifidobacterium bifidum |
| | Bifidobacterium longum |
| | Bifidobacterium kashiwanohense |
| 14 | Bifidobacterium infantis |
| | Bifidobacterium longum |
| | Bifidobacterium kashiwanohense |
| 15 | Bifidobacterium infantis |
| | Bifidobacterium pseudocatenulatum |
| 16 | Bifidobacterium infantis |
| | Bifidobacterium breve |
| | Bifidobacterium pseudocatenulatum |
| 17 | Bifidobacterium infantis |
| | Bifidobacterium breve |
| | Bifidobacterium longum |
| | Bifidobacterium pseudocatenulatum |
| 18 | Bifidobacterium infantis |
| | Bifidobacterium breve |
| | Bifidobacterium bifidum |
| | Bifidobacterium pseudocatenulatum |
| 19 | Bifidobacterium infantis |
| | Bifidobacterium bifidum |
| | Bifidobacterium pseudocatenulatum |
| 20 | Bifidobacterium infantis |
| | Bifidobacterium bifidum |
| | Bifidobacterium longum |
| | Bifidobacterium pseudocatenulatum |
| 21 | Bifidobacterium infantis |
| | Bifidobacterium longum |
| | Bifidobacterium pseudocatenulatum |
| 22 | Bifidobacterium infantis |
| | Limosilactobacillus pontis_A |
| 23 | Bifidobacterium infantis |
| | Bifidobacterium breve |
| | Limosilactobacillus pontis_A |
| 24 | Bifidobacterium infantis |
| | Bifidobacterium breve |
| | Bifidobacterium longum |
| | Limosilactobacillus pontis_A |
| 25 | Bifidobacterium infantis |
| | Bifidobacterium breve |
| | Bifidobacterium bifidum |
| | Limosilactobacillus pontis_A |
| 26 | Bifidobacterium infantis |
| | Bifidobacterium bifidum |
| | Limosilactobacillus pontis_A |
| 27 | Bifidobacterium infantis |
| | Bifidobacterium bifidum |
| | Bifidobacterium longum |
| | Limosilactobacillus pontis_A |
| 28 | Bifidobacterium infantis |
| | Bifidobacterium longum |
| | Limosilactobacillus pontis_A |
| 29 | Bifidobacterium infantis |
| | Lacticaseibacillus rhamnosus |
| 30 | Bifidobacterium infantis |
| | Bifidobacterium breve |
| | Lacticaseibacillus rhamnosus |
| 31 | Bifidobacterium infantis |
| | Bifidobacterium breve |
| | Bifidobacterium longum |
| | Lacticaseibacillus rhamnosus |
| 32 | Bifidobacterium infantis |
| | Bifidobacterium breve |
| | Bifidobacterium bifidum |
| | Lacticaseibacillus rhamnosus |
| 33 | Bifidobacterium infantis |
| | Bifidobacterium bifidum |
| | Lacticaseibacillus rhamnosus |
| 34 | Bifidobacterium infantis |
| | Bifidobacterium bifidum |
| | Bifidobacterium longum |
| | Lacticaseibacillus rhamnosus |
| 35 | Bifidobacterium infantis |
| | Bifidobacterium longum |
| | Lacticaseibacillus rhamnosus |
| 36 | Bifidobacterium infantis |
| | Lacticaseibacillus paracasei |
| 37 | Bifidobacterium infantis |
| | Bifidobacterium breve |
| | Lacticaseibacillus paracasei |
| 38 | Bifidobacterium infantis |
| | Bifidobacterium breve |
| | Bifidobacterium longum |
| | Lacticaseibacillus paracasei |
| 39 | Bifidobacterium infantis |
| | Bifidobacterium breve |
| | Bifidobacterium bifidum |
| | Lacticaseibacillus paracasei |
| 40 | Bifidobacterium infantis |
| | Bifidobacterium bifidum |
| | Lacticaseibacillus paracasei |
| 41 | Bifidobacterium infantis |
| | Bifidobacterium bifidum |
| | Bifidobacterium longum |
| | Lacticaseibacillus paracasei |
| 42 | Bifidobacterium infantis |
| | Bifidobacterium longum |
| | Lacticaseibacillus paracasei |
| 43 | Bifidobacterium infantis |
| | Escherichia coli |
| 44 | Bifidobacterium infantis |
| | Bifidobacterium breve |
| | Escherichia coli |
| 45 | Bifidobacterium infantis |
| | Bifidobacterium breve |
| | Bifidobacterium longum |
| | Escherichia coli |
| 46 | Bifidobacterium infantis |
| | Bifidobacterium breve |
| | Bifidobacterium bifidum |
| | Escherichia coli |
| 47 | Bifidobacterium infantis |
| | Bifidobacterium bifidum |
| | Escherichia coli |
| 48 | Bifidobacterium infantis |
| | Bifidobacterium bifidum |
| | Bifidobacterium longum |
| | Escherichia coli |
| 49 | Bifidobacterium infantis |
| | Bifidobacterium longum |
| | Escherichia coli |
| 50 | Bifidobacterium infantis |
| | Bifidobacterium catenulatum |
| 51 | Bifidobacterium infantis |
| | Bifidobacterium breve |
| | Bifidobacterium catenulatum |
| 52 | Bifidobacterium infantis |
| | Bifidobacterium breve |
| | Bifidobacterium longum |
| | Bifidobacterium catenulatum |
| 53 | Bifidobacterium infantis |
| | Bifidobacterium breve |
| | Bifidobacterium bifidum |
| | Bifidobacterium catenulatum |
| 54 | Bifidobacterium infantis |
| | Bifidobacterium bifidum |
| | Bifidobacterium catenulatum |
| 55 | Bifidobacterium infantis |
| | Bifidobacterium bifidum |
| | Bifidobacterium longum |
| | Bifidobacterium catenulatum |
| 56 | Bifidobacterium infantis |
| | Bifidobacterium longum |
| | Bifidobacterium catenulatum |
| 57 | Bifidobacterium infantis |
| | Bifidobacterium adolescentis |
| 58 | Bifidobacterium infantis |
| | Bifidobacterium breve |
| | Bifidobacterium adolescentis |
| 59 | Bifidobacterium infantis |
| | Bifidobacterium breve |
| | Bifidobacterium longum |
| | Bifidobacterium adolescentis |
| 60 | Bifidobacterium infantis |
| | Bifidobacterium breve |
| | Bifidobacterium bifidum |
| | Bifidobacterium adolescentis |
| 61 | Bifidobacterium infantis |
| | Bifidobacterium bifidum |
| | Bifidobacterium adolescentis |
| 62 | Bifidobacterium infantis |
| | Bifidobacterium bifidum |
| | Bifidobacterium longum |
| | Bifidobacterium adolescentis |
| 63 | Bifidobacterium infantis |
| | Bifidobacterium longum |
| | Bifidobacterium adolescentis |
| 64 | Bifidobacterium infantis |
| | Limosilactobacillus reuteri |
| 65 | Bifidobacterium infantis |
| | Bifidobacterium breve |
| | Limosilactobacillus reuteri |
| 66 | Bifidobacterium infantis |
| | Bifidobacterium breve |
| | Bifidobacterium longum |
| | Limosilactobacillus reuteri |
| 67 | Bifidobacterium infantis |
| | Bifidobacterium breve |
| | Bifidobacterium bifidum |
| | Limosilactobacillus reuteri |
| 68 | Bifidobacterium infantis |
| | Bifidobacterium bifidum |
| | Limosilactobacillus reuteri |
| 69 | Bifidobacterium infantis |
| | Bifidobacterium bifidum |
| | Bifidobacterium longum |
| | Limosilactobacillus reuteri |
| 70 | Bifidobacterium infantis |
| | Bifidobacterium longum |
| | Limosilactobacillus reuteri |
| 71 | Bifidobacterium infantis |
| | Phocaeicola vulgatus |
| 72 | Bifidobacterium infantis |
| | Bifidobacterium breve |
| | Phocaeicola vulgatus |
| 73 | Bifidobacterium infantis |
| | Bifidobacterium breve |
| | Bifidobacterium longum |
| | Phocaeicola vulgatus |
| 74 | Bifidobacterium infantis |
| | Bifidobacterium breve |
| | Bifidobacterium bifidum |
| | Phocaeicola vulgatus |
| 75 | Bifidobacterium infantis |
| | Bifidobacterium bifidum |
| | Phocaeicola vulgatus |
| 76 | Bifidobacterium infantis |
| | Bifidobacterium bifidum |
| | Bifidobacterium longum |
| | Phocaeicola vulgatus |
| 77 | Bifidobacterium infantis |
| | Bifidobacterium longum |
| | Phocaeicola vulgatus |
| 78 | Bifidobacterium infantis |
| | Phocaeicola dorei |
| 79 | Bifidobacterium infantis |
| | Bifidobacterium breve |
| | Phocaeicola dorei |
| 80 | Bifidobacterium infantis |
| | Bifidobacterium breve |
| | Bifidobacterium longum |
| | Phocaeicola dorei |
| 81 | Bifidobacterium infantis |
| | Bifidobacterium breve |
| | Bifidobacterium bifidum |
| | Phocaeicola dorei |
| 82 | Bifidobacterium infantis |
| | Bifidobacterium bifidum |
| | Phocaeicola dorei |
| 83 | Bifidobacterium infantis |
| | Bifidobacterium bifidum |
| | Bifidobacterium longum |
| | Phocaeicola dorei |
| 84 | Bifidobacterium infantis |
| | Bifidobacterium longum |
| | Phocaeicola dorei |
| 85 | Bifidobacterium infantis |
| | Bacteroides fragilis |
| 86 | Bifidobacterium infantis |
| | Bifidobacterium breve |
| | Bacteroides fragilis |
| 87 | Bifidobacterium infantis |
| | Bifidobacterium breve |
| | Bifidobacterium longum |
| | Bacteroides fragilis |
| 88 | Bifidobacterium infantis |
| | Bifidobacterium breve |
| | Bifidobacterium bifidum |
| | Bacteroides fragilis |
| 89 | Bifidobacterium infantis |
| | Bifidobacterium bifidum |
| | Bacteroides fragilis |
| 90 | Bifidobacterium infantis |
| | Bifidobacterium bifidum |
| | Bifidobacterium longum |
| | Bacteroides fragilis |
| 91 | Bifidobacterium infantis |
| | Bifidobacterium longum |
| | Bacteroides fragilis |
| 92 | Bifidobacterium infantis |
| | Bifidobacterium bifidum |
| | Bifidobacterium longum |
| | Bifidobacterium breve |
| 93 | Bifidobacterium infantis |
| | Bifidobacterium bifidum |
| | Bifidobacterium longum |
| | Bifidobacterium breve |
| | Bifidobacterium kashiwanohense |
| 94 | Bifidobacterium infantis |
| | Bifidobacterium bifidum |
| | Bifidobacterium longum |
| | Bifidobacterium breve |
| | Bifidobacterium adolescentis |
| 95 | Bifidobacterium infantis |
| | Bifidobacterium bifidum |
| | Bifidobacterium longum |
| | Bifidobacterium breve |
| | Bifidobacterium pseudocatenulatum |
| 96 | Bifidobacterium infantis |
| | Bifidobacterium bifidum |
| | Bifidobacterium longum |
| | Bifidobacterium breve |
| | Escherichia coli |
| 97 | Bifidobacterium infantis |
| | Bifidobacterium bifidum |
| | Bifidobacterium longum |
| | Bifidobacterium breve |
| | Limosilactobacillus reuteri |
| 98 | Bifidobacterium infantis |
| | Bifidobacterium bifidum |
| | Bifidobacterium longum |
| | Bifidobacterium breve |
| | Limosilactobacillus pontis_A |
| 99 | Bifidobacterium infantis |
| | Bifidobacterium bifidum |
| | Bifidobacterium longum |
| | Bifidobacterium breve |
| | Lacticaseibacillus rhamnosus |
| 100 | Bifidobacterium infantis |
| | Bifidobacterium bifidum |
| | Bifidobacterium longum |
| | Bifidobacterium breve |
| | Lacticaseibacillus paracasei |
| 101 | Bifidobacterium infantis |
| | Bifidobacterium bifidum |
| | Bifidobacterium longum |
| | Bifidobacterium breve |
| | Phocaeicola vulgatus |
| 102 | Bifidobacterium infantis |
| | Bifidobacterium bifidum |
| | Bifidobacterium longum |
| | Bifidobacterium breve |
| | Phocaeicola dorei |
| 103 | Bifidobacterium infantis |
| | Bifidobacterium bifidum |
| | Bifidobacterium longum |
| | Bifidobacterium breve |
| | Bacteroides fragilis |
| 104 | Bifidobacterium infantis |
| | Collinsella sp900759335 |
| 105 | Bifidobacterium infantis |
| | Bifidobacterium breve |
| | Collinsella sp900759335 |
| 106 | Bifidobacterium infantis |
| | Bifidobacterium breve |
| | Bifidobacterium longum |
| | Collinsella sp900759335 |
| 107 | Bifidobacterium infantis |
| | Bifidobacterium breve |
| | Bifidobacterium bifidum |
| | Collinsella sp900759335 |
| 108 | Bifidobacterium infantis |
| | Bifidobacterium bifidum |
| | Collinsella sp900759335 |
| 109 | Bifidobacterium infantis |
| | Bifidobacterium bifidum |
| | Bifidobacterium longum |
| | Collinsella sp900759335 |
| 110 | Bifidobacterium infantis |
| | Bifidobacterium longum |
| | Collinsella sp900759335 |
| 111 | Bifidobacterium infantis |
| | Bifidobacterium bifidum |
| | Bifidobacterium longum |
| | Bifidobacterium breve |
| | Collinsella sp900759335 |
| 112 | Bifidobacterium infantis |
| | Veillonella atypica |
| 113 | Bifidobacterium infantis |
| | Bifidobacterium breve |
| | Veillonella atypica |
| 114 | Bifidobacterium infantis |
| | Bifidobacterium breve |
| | Bifidobacterium longum |
| | Veillonella atypica |
| 115 | Bifidobacterium infantis |
| | Bifidobacterium breve |
| | Bifidobacterium bifidum |
| | Veillonella atypica |
| 116 | Bifidobacterium infantis |
| | Bifidobacterium bifidum |
| | Veillonella atypica |
| 117 | Bifidobacterium infantis |
| | Bifidobacterium bifidum |
| | Bifidobacterium longum |
| | Veillonella atypica |
| 118 | Bifidobacterium infantis |
| | Bifidobacterium longum |
| | Veillonella atypica |
| 119 | Bifidobacterium infantis |
| | Bifidobacterium bifidum |
| | Bifidobacterium longum |
| | Bifidobacterium breve |
| | Veillonella atypica |
| 120 | Bifidobacterium bifidum |
| 121 | Bifidobacterium breve |
| 122 | Bifidobacterium longum |
| 123 | Bifidobacterium infantis |
| | Bifidobacterium catenulatum |
| 124 | Bifidobacterium infantis |
| | Bifidobacterium breve |
| | Bifidobacterium catenulatum |
| 125 | Bifidobacterium infantis |
| | Bifidobacterium breve |
| | Bifidobacterium longum |
| | Bifidobacterium catenulatum |
| 126 | Bifidobacterium infantis |
| | Bifidobacterium breve |
| | Bifidobacterium bifidum |
| | Bifidobacterium catenulatum |
| 127 | Bifidobacterium infantis |
| | Bifidobacterium bifidum |
| | Bifidobacterium catenulatum |
| 128 | Bifidobacterium infantis |
| | Bifidobacterium bifidum |
| | Bifidobacterium longum |
| | Bifidobacterium catenulatum |
| 129 | Bifidobacterium infantis |
| | Bifidobacterium longum |
| | Bifidobacterium catenulatum |
| 130 | Bifidobacterium infantis |
| | Bifidobacterium bifidum |
| | Bifidobacterium longum |
| | Bifidobacterium breve |
| | Bifidobacterium catenulatum |
| 131 | Bifidobacterium infantis |
| | Bifidobacterium bifidum |
| | Bifidobacterium longum |
| | Bifidobacterium breve |
| | Bifidobacterium catenulatum |
| | Bifidobacterium adolescentis |
| | Bifidobacterium pseudocatenulatum |
| | Bifidobacterium kashiwanohense |
| 132 | Bifidobacterium infantis |
| | Bifidobacterium bifidum |
| | Bifidobacterium longum |
| | Bifidobacterium breve |
| | Bifidobacterium catenulatum |
| | Bifidobacterium adolescentis |
| | Bifidobacterium pseudocatenulatum |
| | Bifidobacterium kashiwanohense |
| | Collinsella sp900759335 |
| | Limosilactobacillus pontis_A |

TABLE 3
List of exemplary prebiotics and prebiotic combinations
that can be used with compositions, formulations
and pharmaceutical combinations as provided herein,
and in methods as provided herein:

| | |
|---|---|
| 1 | Lacto-N-tetraose |
| 2 | 2′-fucosyllactose |
| 3 | 3′-sialyllactose |
| 4 | Lacto-N-tetraose |
| | 2′-fucosyllactose |
| 5 | 2′-fucosyllactose |
| | 3′-sialyllactose |
| 6 | Lacto-N-tetraose |
| | 3′-sialyllactose |
| 7 | Lacto-N-tetraose |
| | 2′-fucosyllactose |
| | 3′-sialyllactose |
| 8 | Lacto-N-tetraose |
| | 2′-fucosyllactose |
| | 3′-sialyllactose |
| | Lacto-N-neotetraose |
| | 3-fucosyllactose |
| | 6′-sialyllactose |
| 9 | Lacto-N-tetraose |
| | Porphyran |
| 10 | 2′-fucosyllactose |
| | Porphyran |
| 11 | 3′-sialyllactose |
| | Porphyran |
| 12 | Lacto-N-tetraose |
| | 2′-fucosyllactose |
| | Porphyran |
| 13 | 2′-fucosyllactose |
| | 3′-sialyllactose |
| | Porphyran |
| 14 | Lacto-N-tetraose |
| | 3′-sialyllactose |
| | Porphyran |
| 15 | Lacto-N-tetraose |
| | 2′-fucosyllactose |
| | 3′-sialyllactose |
| | Porphyran |
| 16 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | Porphyran |
| 17 | Lacto-N-tetraose |
| | Fructooligosaccharides |
| 18 | 2′-fucosyllactose |
| | Fructooligosaccharides |
| 19 | 3′-sialyllactose |
| | Fructooligosaccharides |
| 20 | Lacto-N-tetraose |
| | 2′-fucosyllactose |
| | Fructooligosaccharides |
| 21 | 2′-fucosyllactose |
| | 3′-sialyllactose |
| | Fructooligosaccharides |
| 22 | Lacto-N-tetraose |
| | 3′-sialyllactose |
| | Fructooligosaccharides |
| 23 | Lacto-N-tetraose |
| | 2′-fucosyllactose |
| | 3′-sialyllactose |
| | Fructooligosaccharides |
| 24 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | Fructooligosaccharides |
| 25 | Lacto-N-tetraose |
| | Galactooligosaccharides |
| 26 | 2′-fucosyllactose |
| | Galactooligosaccharides |
| 27 | 3′-sialyllactose |
| | Galactooligosaccharides |
| 28 | Lacto-N-tetraose |
| | 2′-fucosyllactose |
| | Galactooligosaccharides |
| 29 | 2′-fucosyllactose |
| | 3′-sialyllactose |
| | Galactooligosaccharides |
| 30 | Lacto-N-tetraose |
| | 3′-sialyllactose |
| | Galactooligosaccharides |
| 31 | Lacto-N-tetraose |
| | 2′-fucosyllactose |
| | 3′-sialyllactose |
| | Galactooligosaccharides |
| 32 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | Galactooligosaccharides |
| 33 | Lacto-N-tetraose |
| | Xylooligosaccharides |
| 34 | 2′-fucosyllactose |
| | Xylooligosaccharides |
| 35 | 3′-sialyllactose |
| | Xylooligosaccharides |
| 36 | Lacto-N-tetraose |
| | 2′-fucosyllactose |
| | Xylooligosaccharides |
| 37 | 2′-fucosyllactose |
| | 3′-sialyllactose |
| | Xylooligosaccharides |
| 38 | Lacto-N-tetraose |
| | 3′-sialyllactose |
| | Xylooligosaccharides |
| 39 | Lacto-N-tetraose |
| | 2′-fucosyllactose |
| | 3′-sialyllactose |
| | Xylooligosaccharides |
| 40 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | Xylooligosaccharides |
| 41 | Lacto-N-tetraose |
| | Mucin |
| 42 | 2′-fucosyllactose |
| | Mucin |
| 43 | 3′-sialyllactose |
| | Mucin |
| 44 | Lacto-N-tetraose |
| | 2′-fucosyllactose |
| | Mucin |
| 45 | 2′-fucosyllactose |
| | 3′-sialyllactose |
| | Mucin |
| 46 | Lacto-N-tetraose |
| | 3′-sialyllactose |
| | Mucin |
| 47 | Lacto-N-tetraose |
| | 2′-fucosyllactose |
| | 3′-sialyllactose |
| | Mucin |
| 48 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | Mucin |
| 49 | Lacto-N-tetraose |
| | 2′-fucosyllactose |
| | 3′-sialyllactose |
| | 6′-sialyllactose |
| | 2′,3-di-fucosyllactose |
| | Porphyran |
| 50 | Lacto-N-tetraose |
| | 2′-fucosyllactose |
| | 3′-sialyllactose |
| | 6′-sialyllactose |
| | 2′,3-di-fucosyllactose |
| | Fructooligosaccharides |
| 51 | Lacto-N-tetraose |
| | 2′-fucosyllactose |
| | 3′-sialyllactose |
| | 6′-sialyllactose |
| | 2′,3-di-fucosyllactose |
| | Galactooligosaccharides |
| 52 | Lacto-N-tetraose |
| | 2′-fucosyllactose |
| | 3′-sialyllactose |
| | 6′-sialyllactose |
| | 2′,3-di-fucosyllactose |
| | Xylooligosaccharides |
| 53 | Lacto-N-tetraose |
| | 2′-fucosyllactose |
| | 3′-sialyllactose |
| | 6′-sialyllactose |
| | 2′,3-di-fucosyllactose |
| | Mucin |
| 54 | Lacto-N-tetraose |
| | 2′-fucosyllactose |
| | 3′-sialyllactose |
| | 3-fucosyllactose |
| | 6′-sialyllactose |
| | Porphyran |
| 55 | Lacto-N-tetraose |
| | 2′-fucosyllactose |
| | 3′-sialyllactose |
| | 3-fucosyllactose |
| | 6′-sialyllactose |
| | Fructooligosaccharides |
| 56 | Lacto-N-tetraose |
| | 2′-fucosyllactose |
| | 3′-sialyllactose |
| | 3-fucosyllactose |
| | 6′-sialyllactose |
| | Galactooligosaccharides |
| 57 | Lacto-N-tetraose |
| | 2′-fucosyllactose |
| | 3′-sialyllactose |
| | 3-fucosyllactose |
| | 6′-sialyllactose |
| | Xylooligosaccharides |
| 58 | Lacto-N-tetraose |
| | 2′-fucosyllactose |
| | 3′-sialyllactose |
| | 3-fucosyllactose |
| | 6′-sialyllactose |
| | Mucin |
| 59 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | 2′,3-di-fucosyllactose |
| | Porphyran |
| 60 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | 2′,3-di-fucosyllactose |
| | Fructooligosaccharides |
| 61 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | 2′,3-di-fucosyllactose |
| | Galactooligosaccharides |
| 62 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | 2′,3-di-fucosyllactose |
| | Xylooligosaccharides |
| 63 | Lacto-N-tetraose |
| | 2′-fucosyllactose |
| | 3′-sialyllactose |
| | Lacto-N-neotetraose |
| | 3-fucosyllactose |
| | 6′-sialyllactose |
| | 2′,3-di-fucosyllactose |
| | Mucin |
| 64 | Porphyran |
| 65 | Fructooligosaccharides |
| 66 | Galactooligosaccharides |
| 67 | Xylooligosaccharides |
| 68 | Mucin |
| 72 | Lacto-N-neotetraose |
| 73 | 3-fucosyllactose |
| 74 | 6′-sialyllactose |
| 75 | 2′,3-di-fucosyllactose |

### Example 5: Isolation and Identification of Pure Microbial Strains from Fecal Matter

### Strain Isolation

In alternative embodiments, microbes used in compositions, formulations and pharmaceutical combinations as provided herein, or used to practice methods as provided herein, are isolated from fecal matter, and can be used in the form of a pure microbial strain isolated from fecal matter.

Individual bacterial strains can be isolated and cultured from fecal matter material for further study and for assembly of probiotics and/or therapeutic biologicals, i.e., for manufacturing combinations of microbes as provided herein. Most live bacteria that inhabit fecal matter tend to be obligate anaerobes so care must be taken to perform all culture and isolation work in the anaerobic chamber to prevent their exposure to oxygen, and to use various anaerobic growth media that includes reductant compounds as described in Example 1. Growth media and plates that favor growth of target bacteria can be used to improve the ability to find and isolate them as pure living cultures. To isolate Bifidobacterium specifically, Bifidobacterium Selective Agar can be used. Different anaerobic growth media are used to enable growth of different subsets of microbes to improve overall ability to isolate and purify an inclusive number of unique bacterial species from each individual fecal material sample.

To begin a microbial isolation and characterization campaign, one cryotube containing cryogenically preserved fecal matter is removed from storage in the liquid nitrogen Dewar, brought into the anaerobic chamber, and then allowed to thaw gently on ice. The entire 1 ml contents are added to 10 ml of Anaerobe Basal Broth (ABB) or another suitable anaerobic growth medium to establish a 1/10 dilution. Successive 10-fold serial dilutions are then performed in ABB to establish 1/100, 1/1000, 1/10000, 1/100000, 1/1000000 dilutions of the fecal matter. From each of the 1/10000, 1/100000, and 1,1000000 dilutions, four 0.1 ml volumes are removed and then added to and spread over solid anaerobic growth medium of choice. The platings are incubated at 37° C. for 1, 2, 3, 4, 5, 6, 7, 8, 9, 10, 11, 12 days to allow for a wide variety of bacterial colonies to grow. Typically, plates are evaluated following 1-2 days of growth. Platings are made from several liquid dilutions of fecal matter to ensure that there will be ones that have numerous yet non-overlapping colonies for efficient colony picking.

Colonies are manually picked from plates using sterile pipette tips. Colonies may also be picked by an automated colony picking machine that is enclosed in an anaerobic chamber. Colonies are picked in multiples of 96 to accommodate subsequent 96-well-based genomic DNA isolation steps and large-scale cryogenic storage steps. After visible colonies are evident on the streak, single colonies are picked and then inoculated into an individual well of a 2 ml 96-well deep well block, each well with 1 ml liquid anaerobic growth medium of choice. Once all wells of the deep-well block have been inoculated with different picked colonies, the deep well block is covered with an adhesive gas-permeable seal and then incubated at 37° C. in an incubator within the anaerobic chamber for 1, 2, 3, 4, 5, 6, 7, 8, 9, 10, 11, 12 days to allow for liquid growth from each isolated colony. Typically, 96 well plates are harvested after 1-2 days of growth.

After turbid growth is apparent in all wells, the gas-permeable seal is removed from the 96-well deep well block and a viable stock representation is made by transferring 0.1 ml culture from each well to the corresponding wells of a second 96-well deep-well block, each well containing 0.4 ml of the same anaerobic growth medium plus 0.5 ml Biobank Buffer (Phosphate Buffered Saline plus 2% Trehalose plus 10% dimethyl sulfoxide. The volumes in each well are thoroughly mixed by pipetting up and down several times, then the deep-well block is sealed with an impermeable foil seal rated for −80° C. storage and stored in a −80° C. freezer.

### Sequence and Computational Characterization of Isolated Fecal Bacteria

The remaining 0.9 ml culture in the original 96-well deep-well plate is then used for whole genome sequence determination of the isolated strain as follows: The deep-well block is subjected to centrifugation for 20 minutes at 6000 g to pellet the cells. After centrifugation, 0.8 ml supernatant is carefully removed by pipette, leaving 0.1 ml pellet and medium for gDNA processing. Total genomic DNA is extracted from the cell pellet using the MAGATTRACT POWERMICROBIOME DNA/RNA EP KIT™ (Qiagen). Genomic DNA is then prepared for Whole Genome Sequencing analysis using the KAPA HYPERPLUS KIT™ (Roche). Sequencing analysis is conducted on the Illumina platform using paired-end 150 bp reads.

Sequencing data is first processed to remove low quality reads and adapter contamination using Trim Galore, a wrapper for cutadapt.

Microbial and archaeal assembled genomes from the Genome Taxonomy Database (GTDB) (Parks et al. (2019) bioRxiv 771964, Méric et al. (2019) bioRxiv 712166) were used as a reference for classification using CENTRIFUGE™ (Kim et al. (2016) Genome Research 26:1721-1729). CENTRIFUGE™ classifies sequencing reads from a metagenomic fecal sample to reference sequences and uses an expectation-maximization method to estimate relative abundance of the taxa present in the sample.

Unique strains (Table 4) were isolated from the MY BABY BIOME™ study using the described methodology.

TABLE 4
Exemplary bacterial strains used in formulations and pharmaceutical combinations
as provided herein which are isolated from human fecal material, and optionally
that can be used alone to practice methods as provided herein, or in making
or using combinations, mixes or consortia of microbe compositions as provided
herein; listed are the closest genome/species matches for each strain,
determined by the analysis described herein:

| | | | | Percent of Reads |
|---|---|---|---|---|
| | | NCBI | | Mapping to |
| | Screening | Taxonomy | | Reference |
| Strain ID | Medium | ID | NCBI Name | Assembly |
| PBI-001 | ABB | 1208091 | Enterococcus gallinarum | 78% |
| | | | NBRC 100675 | |
| PBI-002 | ABB | 391904 | Bifidobacterium longum | 74% |
| | | | subsp. infantis ATCC 15697 | |
| PBI-003 | ABB | 518634 | Bifidobacterium breve DSM | 85% |
| | | | 20213 | |
| PBI-004 | ABB | 197614 | Streptococcus pasteurianus | 77% |
| PBI-005 | ABB | 500634 | Bifidobacterium bifidum ATCC | 98% |
| | | | 29521 | |
| PBI-006 | ABB | 317010 | Enterococcus canintestini | 100% |
| PBI-007 | ABB | 866789 | Escherichia coli DSM 30083 | 54% |
| PBI-008 | ABB | 565042 | Bifidobacterium longum subsp. | 82% |
| | | | longum JCM 1217 | |
| PBI-009 | ABB | 391904 | Bifidobacterium longum subsp. | 74% |
| | | | infantis ATCC 15697 | |
| PBI-010 | ABB | 1169286 | Enterococcus faecalis ATCC | 94% |
| | | | 19433 | |
| PBI-011 | ABB | 208479 | Enterocloster bolteae | 58% |
| PBI-012 | ABB | 518634 | Bifidobacterium breve DSM | 95% |
| | | | 20213 | |
| PBI-013 | ABB | 479437 | Eggerthella lenta DSM 2243 | 91% |
| PBI-014 | ABB | 411490 | Anaerostipes caccae L1-92 | 97% |
| PBI-015 | ABB | 500634 | Bifidobacterium bifidum ATCC | 97% |
| | | | 29521 | |
| PBI-016 | ABB | 1208091 | Enterococcus gallinarum | 45% |
| | | | NBRC 100675 | |
| PBI-017 | ABB | 518634 | Bifidobacterium breve DSM | 94% |
| | | | 20213 | |
| PBI-018 | ABB | 1169286 | Enterococcus faecalis ATCC | 95% |
| | | | 19433 | |
| PBI-019 | ABB | 1282 | Staphylococcus epidermidis | 95% |
| PBI-020 | ABB | 565042 | Bifidobacterium longum subsp. | 84% |
| | | | longum JCM 1217 | |
| PBI-021 | ABB | 866789 | Escherichia coli DSM 30083 | 35% |
| PBI-022 | ABB | 391904 | Bifidobacterium longum subsp. | 81% |
| | | | infantis ATCC 15697 | |
| PBI-023 | ABB | 518634 | Bifidobacterium breve DSM | 93% |
| | | | 20213 | |
| PBI-024 | ABB | 500634 | Bifidobacterium bifidum ATCC | 98% |
| | | | 29521 | |
| PBI-025 | ABB | 1169286 | Enterococcus faecalis ATCC | 96% |
| | | | 19433 | |
| PBI-026 | ABB | 47715 | Lacticaseibacillus rhamnosus | 92% |
| PBI-027 | ABB | 47714 | Lacticaseibacillus paracasei | 94% |
| | | | subsp. paracasei | |
| PBI-028 | ABB | 866789 | Escherichia coli DSM 30083 | 55% |
| PBI-029 | ABB | 866789 | Escherichia coli DSM 30083 | 55% |
| PBI-030 | ABB | 1282 | Staphylococcus epidermidis | 95% |
| PBI-031 | ABB | 391904 | Bifidobacterium longum subsp. | 73% |
| | | | infantis ATCC 15697 | |
| PBI-032 | ABB | 1280 | Staphylococcus aureus | 98% |
| PBI-033 | ABB | 391904 | Bifidobacterium longum subsp. | 75% |
| | | | infantis ATCC 15697 | |
| PBI-034 | ABB | 518634 | Bifidobacterium breve DSM | 95% |
| | | | 20213 | |
| PBI-035 | ABB | 565042 | Bifidobacterium longum subsp. | 90% |
| | | | longum JCM 1217 | |
| PBI-036 | ABB | 47715 | Lacticaseibacillus rhamnosus | 99% |
| PBI-037 | ABB | 1028307 | Klebsiella aerogenes KCTC 2190 | 94% |
| PBI-038 | ABB | 565042 | Bifidobacterium longum subsp. | 88% |
| | | | longum JCM 1217 | |
| PBI-039 | ABB | 866789 | Escherichia coli DSM 30083 | 68% |
| PBI-040 | ABB | 518634 | Bifidobacterium breve DSM | 96% |
| | | | 20213 | |
| PBI-041 | ABB | 1169286 | Enterococcus faecalis ATCC | 94% |
| | | | 19433 | |
| PBI-042 | ABB | 518634 | Bifidobacterium breve DSM | 93% |
| | | | 20213 | |
| PBI-043 | ABB | 565042 | Bifidobacterium longum subsp. | 90% |
| | | | longum JCM 1217 | |

In alternative embodiments, complete genomes are generated for organisms of special interest using long-read sequencing. High molecular weight genomic DNA is prepared from organisms of interest using a commercially available kit for example Genomic-tip (Qiagen). Library preparation on genomic DNA is performed using the NATIVE BARCODING KIT 24 V14™ (Oxford Nanopore) and sequencing is performed on a MINION™ (Oxford Nanopore). Reads are filtered and trimmed for quality and assembly is performed using the assembler FLYE™ (Kolmogorov et al. (2019) Nature Biotechnology 37:540-546). The resulting assembly is polished using MEDAKA™ (Oxford Nanopore Technologies) with short reads to correct for errors inherent in long read sequencing. Genes are predicted on the polished genome using prodigal (Hyatt et al. (2010) BMC Bioinformatics 11:119), the NCBI Prokaryotic Gene Annotation Pipeline (Tatusova et al. (2016) Nucleic Acids Res. 44 (14): 6614-24) or DIAMOND™ (Buchfink et al. (2021) Nature Methods 18:366-68).

In alternative embodiments strain level differences are determined via pangenomic analysis, which uses complete genome sequences to compare the entire set of genes from strains within a clade. Here, a pangenomic analysis was done using 10 complete Bifidobacterium infantis genomes from NCBI and the assembled genome from a B. infantis isolate (PB-STR-093). 48% of the pangenome comprising 1620 gene clusters is the core genome conserved among the strains, shown as continuous bars in FIG. 24. The remainder are accessory genes present in some but not all strains. When the genomes themselves are clustered according to the number and identity of gene clusters they share, they segregate into 2 groups that are distinguished by shared blocks of gene clusters. The region of 13 genes unique to PB-STR-093, not found in the other genomes is highlighted in medium shaded grey in the SCG Clusters band. 7 out of these gene clusters are predicted to be involved in Carbohydrate Transportation and Metabolism (COG20_Category), including 4 xylose transporters.

Strain Definitions for Isolated Strains from Fecal Matter

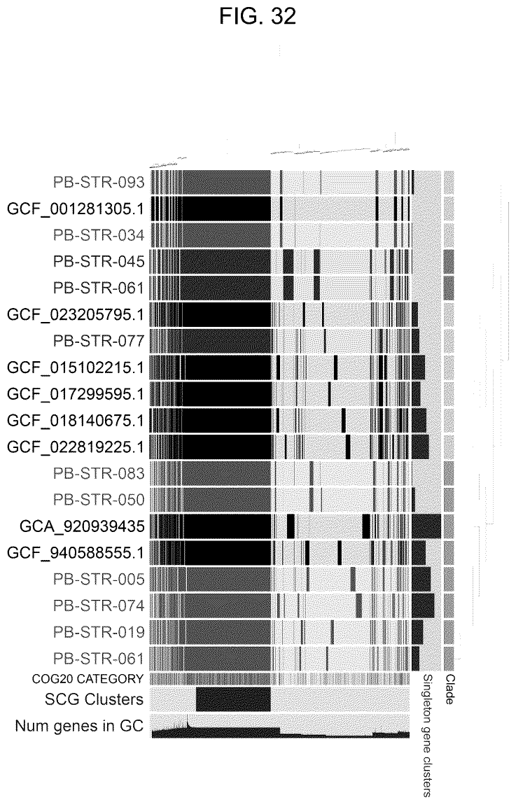

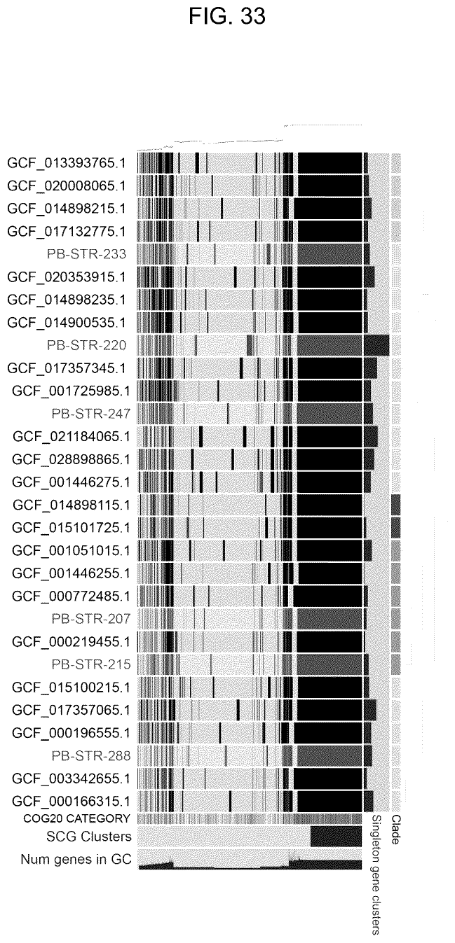

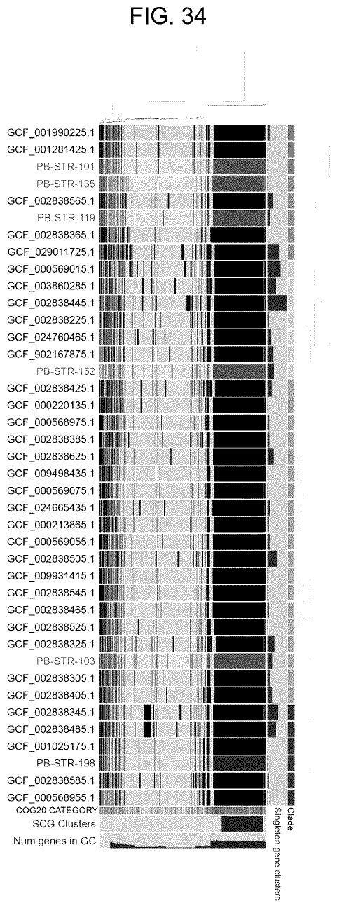

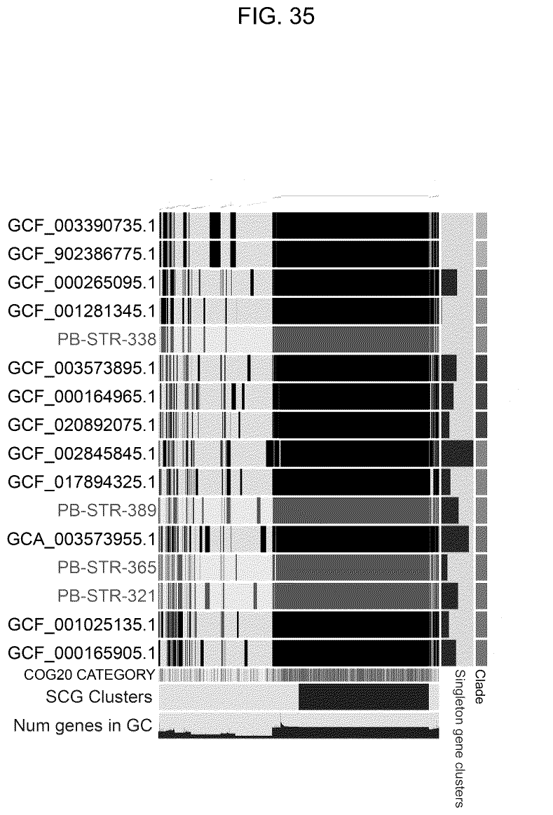

To determine suitable definitions of unique strains, we downloaded publicly available genomes for strains of B. infantis, B. Breve, B. longum, and B. Bifidum from NCBI genome and the Genome Taxonomy Database (GTDB) and compared those genomes to Persephone strains. PB-STR-321, PB-STR-093, PB-STR-083, PB-STR-119, PB-STR-103, PB-STR-207, PB-STR-215, and PB-STR-220 (all isolated and ran through long read sequencing as described above). The downloaded genome accession ids (GenBank or NCBI RefSeq assembly) are listed in Table 28. ANVI'O™ (or Anvi'o; Eren A M et al. (2020) Nature Microbio 6:3-6) was used to generate functional dendrograms from the pangenomic analysis of each species. These dendrograms are shown in FIG. 32 (B. infantis), FIG. 33 (B. longum), FIG. 34 (B. breve), and FIG. 35 (B. bifidum). Similarities between genomes are calculated using ANIb (Goris et al. (2007) Int J Syst Evol Micr 57:81-91) through the pyani.anib module (Pritchard et al. (2016) Anal. Methods, 8:12-24) which gives 3 useful metrics: average nucleotide identity (ANI), genome coverage, and product of ANI and genome coverage.

TABLE 28
Accession IDs (GenBank or NCBI RefSeq assembly)
of genomes used for comparative genomics:

| Species | Accession IDs of Genomes |
|---|---|
| B. | GCF_000166315.1, GCF_003342655.1, GCF_015100215.1, |
| longum | GCF_017357065.1, GCF_028898865.1, GCF_014898115.1, |
| | GCF_014898135.1, GCF_017132775.1, GCF_021184065.1, |
| | GCF_000196555.1, GCF_001446275.1, GCF_020353915.1, |
| | GCF_014900535.1, GCF_014898215.1, GCF_013393765.1, |
| | GCF_015102035.1, GCF_017357325.1, GCF_014898235.1, |
| | GCF_015101725.1, GCF_020008065.1, GCF_000196575.1, |
| | GCF_001051015.1, GCF_001446255.1, GCF_000219455.1, |
| | GCF_000772485.1, GCF_017357345.1, GCF_001725985.1, |
| | GCF_000092325.1. |
| B. | GCF_026967535.1, GCF_023208035.1, GCF_023205815.1, |
| infantis | GCF_023208075.1, GCF_023208115.1, GCF_023205835.1, |
| | GCF_023208055.1, GCF_023208095.1, GCF_023205795.1, |
| | GCF_023208135.1, GCF_023208155.1, GCA_920939435.1, |
| | GCF_940588555.1, GCF_022819225.1, GCF_024665655.1, |
| | GCF_018140675.1, GCF_023205855.1, GCF_015102215.1, |
| | GCF_017378625.1, GCF_017299595.1, GCF_001281305.1, |
| | GCF_000269965.1, GCF_900637215.1, GCF_000020425.1, |
| | GCF_902167885.1, |
| B. | GCF_000213865.1, GCF_024665435.1, GCF_001281425.1, |
| breve | GCF_902167875.1, GCF_002838585.1, GCF_002838225.1, |
| | GCF_002838445.1, GCF_002838505.1, GCF_002838705.1, |
| | GCF_002838725.1, GCF_003813065.1, GCF_001025175.1, |
| | GCF_000568975.1, GCF_000569015.1, GCF_009931415.1, |
| | GCF_009498435.1, GCF_013267755.1, GCF_001990225.1, |
| | GCF_902387425.1, GCF_000569035.1, GCF_900637145.1, |
| | GCF_002838245.1, GCF_002838265.1, GCF_002838285.1, |
| | GCF_002838745.1, GCF_002838325.1, GCF_002838305.1, |
| | GCF_002838605.1, GCF_002838625.1, GCF_002838645.1, |
| | GCF_002838665.1, GCF_002838685.1, GCF_002838365.1, |
| | GCF_002838405.1, GCF_002838385.1, GCF_002838425.1, |
| | GCF_002838345.1, GCF_000569075.1, GCF_002838465.1, |
| | GCF_002838545.1, GCF_024760465.1, GCF_000568955.1, |
| | GCF_002838565.1, GCF_002838525.1, GCF_002838485.1, |
| | GCF_000569055.1, GCF_000220135.1, GCF_029011725.1, |
| | GCF_003860285.1 |
| B. | GCF_001025135.1, GCF_001281345.1, GCF_000265095.1, |
| bifidum | GCF_017894325.1, GCF_016838705.1, GCA_003573955.1, |
| | GCF_902386775.1, GCF_900637095.1, GCF_002845845.1, |
| | GCF_000165905.1, GCF_000164965.1, GCF_003390735.1, |
| | GCF_003573895.1, GCF_029011515.1, GCF_020892075.1 |

PB-STR-220: B. longum

Persephone strain PB-STR-220 is a member of the species B. longum. Comparative genomic analysis of PB-STR-220 was done with the published B. longum genomes listed in Table 28. The type-strain of B. longum is GCF_000196555.1. PB-STR-220 is differentiated from the type-strain by the following values:
accession: GCF_000196555.1,
ani: 98.0%,
coverage: 74.2%,
product: 72.7%,

The most similar published genome to PB-STR-220 is GCF_013393765.1 (determined by the strain with the highest ANIb product). PB-STR-220 is differentiated from GCF_013393765.1 by the following values:
accession: GCF_013393765.1,
ani: 98.0,
coverage: 78.7%,
product: 77.1%,

Table 10 provides a list of the unique open reading frames (ORFs) from PB-STR-220. These ORFs were determined to be unique by BLAST searches with the ORFs from the above list of published B. longum genomes. If an ORF from PB-STR-220 has no corresponding ORF in any of the published genomes (with a sequence identity greater than 60%) it is considered unique and included in the table. If an ORF had a sequence identity greater than 20% but less than 60%, the highest percent sequence identity (pident) is listed. Where functional annotations were possible, they are included in the table.

TABLE 10

| | SEQ | | | |
|---|---|---|---|---|
| | ID | | | |
| index | NO | AA Sequence | pident | function annotations |
| 1 | 1 | MSGTRQYHRLSAETLDTLLRLISEDELTPK | | |
| | | QIAERAGVPRQKVYEYRKKLKDRRKSAPLT | | |
| | | DISTLVIHQRVVFRPDATIENPEDVNGPSF | | |
| | | IDPDSGFDCSRCGQSMSRDWFTIQGNLIKP | | |
| | | DFGYCPGCGGVATPYRDDTINPDAREAGDE | | |
| 2 | 2 | MKFHRISPCPRCGGKVRAKWERDEVLALPE | 41.7 | |
| | | YTFFIVMFRCTACGLSLDGGCSRKPAPYQL | | |
| | | QRSIVVWNRVCNGDKCFTLLYKILAGGR | | |
| 3 | 3 | MSATILDPACGGRMFWFDKHDPRVLFGDCR | | ['Biotin biosynthesis', |
| | | DESWELCDGRRFDVKPDQLMDYRHLPFPDD | | 'Ubiquinone\Vmenaquinone |
| | | SFRLVVLDPPHIRHGGRTSYMVRKYGLLDE | | biosynthesis C- |
| | | HGWPDDLTSMFAECFRVLEPSGILIFKWNE | | methylase UbiE\MenG |
| | | TQIPVSQVLACTPQHPLFGNKQPKQTGTHW | | (UbiE) (PDB:4OBW)', |
| | | IVFMKEALDEIPQD | | 'Coenzyme transport and |
| | | | | metabolism'] |
| 4 | 4 | MNNLIHCDMCGYLMTKRWSETIDGKTYCRD | | |
| | | CVPKKRLIDSGEPTEFDDTDEIVCPYCGHR | | |
| | | YEDSYECGGNDEYFEEECEDCGREFNVTRI | | |
| | | IDISYDTKPKEATEE | | |
| 5 | 5 | MSEPEQNWPESEPDKSKPEMTQEELQRQLL | | |
| | | ETQKRLVELQEQQIRKKDSQTDNHDTGIGK | | |
| | | SVVSILIAAVIIAVVGFVIHDYTKTKEVNE | | |
| | | HASDNISDALNLGSGSPTDIFLSSLPMKP | | |
| 6 | 6 | MSSPVYQPMPPAGMQPSQPVSHRRKKGITI | | |
| | | PVPVFVLLIIGIIVALFVGMLFGVGAMSEE | | |
| | | VNSAQQSSYAWEDKYKAAKVQLNGYVSAPS | | |
| | | VAPDTMGACGVLFDGNQSLIDDVAAVSGYF | | |
| | | RDPANHDAVQAFSGASLAVKTINEAFPKAD | | |
| | | PDMKASLAALNAPMLKIVYATQNLGYADAQ | | |
| | | YDSLQVLSDLNSVMESCVAVGYTAKQ | | |
| 7 | 7 | MVMGSAENHPQSRYARQLPPAGASTGKEDM | | |
| | | RATLETVSCGELTAVYRKDSDTGIVELASW | | |
| | | IVDASSVL | | |
| 8 | 8 | MTFAYGDDRPVAVQSVQMNDVKTRFDRTTP | 39.1 | ['Melibiase', 'Alpha- |
| | | IAEIITTGDGHVPAGNRLTHTGIGLALRYR | | galactosidase (GalA) |
| | | GHRATVEGTRHTLAVTLGDDVRGIEALVRY | | (PDB:2XN0)', |
| | | EVDERVAMVRTDVTVTNTGAAPLLVDFVTS | | 'Carbohydrate transport |
| | | WSSAFGAPEGVVPDARAWDLFEGRSDWLAE | | and metabolism', 'alpha- |
| | | GRWSRRPVNDLLPVISQELTGVDPRQGHQV | | galactosidase |
| | | VSSGTWSTGMHLPLAVLESKTFGLAWLFQV | | [EC:3.2.1.22]'] |
| | | EHNGAWRWDVEDDTVDGGIALSGPTNENHG | | |
| | | WCRDLKPGESFTTVPASFTLAGDEDAAVRR | | |
| | | VTDYRRVMRAPHPDHAVTRTVENDYMNTIN | | |
| | | GDPTTEKELPLIKAAGETGVEIFVIDCGWY | | |
| | | DDSGDWWPSVGEWMPSKTRFPGEKGIVEVV | | |
| | | DAIKAAGMVPGIWLEPEVVGVDSPVAKRLP | | |
| | | DSAFFQRRGRRVMEHSRYLLDFRDPVARAH | | |
| | | VDAVVDRLVTEYGIGYFKFDYNVSPGSGTD | | |
| | | YDADAPGDGLLGHNRAYSAWIESLHRRYPD | | |
| | | LILENCSSGGMREDFAQTGRFQVQSTSDQQ | | |
| | | DWRLYPVIAAAAPMMVLPEQAASWAYPQSD | | |
| | | MTDEETAFNINTTFLGRFFLSGYLNRMDAG | | |
| | | QLDIVRQGVKAYRRHVQPVIGQSVPFWPLG | | |
| | | LPGWDDPLLAYGLDCGDTALVTVWNRGGEG | | |
| | | GDVRLDLRRWRGRAGAVAAVYPTEGFESWP | | |
| | | VHWDAGRGELVVRVPAGVYVSRTFEISFAP | | |
| | | RS | | |
| 9 | 9 | MASRVTLNDIAAAANVSKATVSKALNDTGQ | 51.1 | ['Periplasmic binding |
| | | LSARTRRVVLAAADRLGYVRPRPMTRSRSG | | protein-like domain', |
| | | LIGLVSADLDGRFATPALTGAENTLGAASH | | 'DNA-binding |
| | | AVLLTNSRGDPKLERAHIDQLAARGVDGLL | | transcriptional regulator, |
| | | MLGGETDARPPVRPSTALDIPIVYTYAPST | | LacI\VPurR family (PurR) |
| | | DPNDCSVTCDNVAAGAAAIDHLLSRGRRRI | | (PDB:1BDH)', |
| | | AIIAGPEYYQATKDRLVGAARAMKVAGIRL | | 'Transcription', 'LacI family |
| | | AVPTRYGNWHESWGRTATNLILESGVPIDG | | transcriptional regulator'] |
| | | IYCLNDMLARGAIETLMDCGLDVPRDVAVI | | |
| | | GHDNWAVTATEGPVPITSFDSNLQEIGRRS | | |
| | | ARLLLDIIRGNPRHGTMLIGCSLVVRQSTV | | |
| | | AR | | |
| 10 | 10 | MNVHFSGTAKRIVASIAAVAALGSMAACGS | 33.6 | ['Bacterial extracellular |
| | | NGASNGGSDDELTVSYWDDEQDSIKEFIKQ | | solute-binding protein', |
| | | NPDIKVKEIRVPGDDYNTKLNQMIVGNTAP | | 'ABC-type glycerol-3- |
| | | DVMLVQEADYVRFAQNGVLEKLDDQLSDLG | | phosphate transport |
| | | IDKDDFQPAVKGITNQVDGYYGFPQGFATE | | system, periplasmic |
| | | IMYYNKDLFDAAGVEYPTNDWTWDDYTAAA | | component (UgpB) |
| | | EKLTDASTGQYGSDSPTENGVWYSLIGAAG | | (PDB:2Z8D)', |
| | | DDVVKDGKLSFGNGLKETLEFQKNLVDNKW | | 'Carbohydrate transport |
| | | QPEPASGSKVSDMFAAGKAAMTMGGTWLVS | | and metabolism', 'multiple |
| | | TYKDADENWDIATIPTPEGGRKYNSLHTSF | | sugar transport system |
| | | WTISKNSKHKDAAKKLVKFLMSKEGQKAMS | | substrate-binding protein'] |
| | | QQLGNAPAFQSMMSDGYYRVEGKHGPSNWD | | |
| | | VLTQSTEEARLGYTMVSSTPTEDLYDQFNA | | |
| | | YVLGQTSLDEVTGAQVDKANKEITDAQ | | |
| 11 | 11 | MHSDTGAAWGFLSPWIIGFLVFSAFPLAFS | 43.8 | ['Binding-protein- |
| | | FYLSLTKWNLMGDPQFVGLQNYKDMLSGQG | | dependent transport |
| | | ELGQTLLATFIFTVINVAVSILESLLLAVL | | system inner membrane |
| | | LNFKVRFKGLFQFFYYVPTIMPSVVMAGCL | | component', 'ABC-type |
| | | VLMFNPQLGIINYVLKCIGVENPPNWGGSQ | | sugar transport system, |
| | | TFVWVMVAVASIFTFQTGQQMLVFSAALKD | | permease component |
| | | VPQELYEAAALDGAGAWKRFIHITIPGIAP | | (UgpA) (PDB:3FH6)', |
| | | MMLFNVVSCTVNSENSAFSLLYPLTGGGPG | | Carbohydrate transport |
| | | NATKVIGLLIYDKAFKSFNMGQASALSVIL | | and metabolism', 'multiple |
| | | FIIVGLISILQFRLMDRK | | sugar transport system |
| | | | | permease protein'] |
| 12 | 12 | MYHTPKYVKVLQYIAMILMALFMFFPIYWI | 38.1 | ['ABC-type glycerol-3- |
| | | FVNSLKTINGISAWPPEFFPSDPQWGNYIE | | phosphate transport |
| | | VLKNPNTLLYLRNTLILVVENTLGTLLTSA | | system, permease |
| | | LVAYPLARMHFKGRGVIFGIILATMMVPSA | | component (UgpE) |
| | | ALVIPQYLLFRSFGMLDSFWPLILPSFFAQ | | (PDB:1VR4)', |
| | | PYNVFLFRQFFVSIPESIDEAAMLDGCSRW | | Carbohydrate transport |
| | | QAYWRVIVPLGKPIFITVGIMSASFWWNEL | | and metabolism', 'multiple |
| | | FSPLVYINSEDLKPLTLGVLTSFVQTSAGA | | sugar transport system |
| | | SKTMWNLQMAFSMLMIIPPALMYIFCSKYI | | permease protein'] |
| | | TEGIKTSGMKD | | |
| 13 | 13 | MTDQTTSPKVTITSRFWHRYRTMTVDNALP | 49.1 | ['Beta-L- |
| | | YQWRALNDEVPVDVPEGAAWGENGSQFSHS | | arabinofuranosidase, |
| | | LRNLRIAAGRESGAFSGQPFQDTDVSKWLE | | GH127', 'Beta-L- |
| | | AASYTLRMRDEGFDIDDIEAKVDEAIGLFE | | arabinofuranosidase, |
| | | DLQDEDGYLDTKFEIDLPADQRFKGLRWSH | | GH127 family (HybA1) |
| | | ELYTMGHFIEAAVAHYESTGSPRALDIAER | | (PDB:3WKW) |
| | | AADCIDRHFGDAAGQIHGPDGHPEIELALA | | (PUBMED:24385433)', |
| | | RLYEVTGERRWLDLAAWFIRVRGIDPEFYD | | 'Carbohydrate transport |
| | | EQDKAGGPQFYTDMHMPLKYFVADEPILDK | | and metabolism', 'non- |
| | | AKAEGHAVRLLYLAVAVAKVGRLLEDRKML | | reducing end beta-L- |
| | | DTAERLWRNIVDHRMYVTGAVGSTQVGEAF | | arabinofuranosidase |
| | | SFDDDLPNDLVYGETCASVAMLFYGKALME | | [EC:3.2.1.185]'] |
| | | IRPRGSVADVMELELFNGMLSGIQLDGTRY | | |
| | | FYVNPLEADPAASAGNPTRHHVLTRRAGWF | | |
| | | DCACCPANLVRLITSLDRYLYTESGDTIYA | | |
| | | HQFIANRAEFADGLTVEQTQAGEEYPWSGD | | |
| | | ITFHVANPNRLNKRLAVRIPAWSPRWTLEV | | |
| | | NGNPVNLKAADGFVSIDVSGETTEIHLVLD | | |
| | | MAVRKVRASLDVRCDVGRLAVARGPIVFCM | | |
| | | EQCDNEGPLWLDGMSVDAEVEERYDADLLD | | |
| | | GVEVLTVEGRRFETQRTGQYYTAEAPLAEH | | |
| | | EQQLTLIPYYAWCNRAEGQMQVWVRETR | | |
| 14 | 14 | MRSPKPLAALAAAAVLGGGLAFGAAVPAYA | | |
| | | IENPTVTVHLDQKGCTENSNNTDCQIFHGS | | |
| | | TGFLYGLTDDGISSDTTLAGLNLDEDSVHV | | |
| | | GKSPNGVQHPNGDVMNTTDQWKRNGGGEIQ | | |
| | | VYMKEAYEGFPYAAYGDGSINSYVEKVKTM | | |
| | | VQTFNDKYPEYKDDIVWIPFNEPDISDQNY | | |
| | | YNLTNYSSQYDSVRTRFFEDWDKVVSAIRE | | |
| | | VYPAARIGGPNNSGWNNTFYRDFFNHAKAN | | |
| | | GTVPDVVTWHELGSGFGSYLSNFQQWKALE | | |
| | | KTILDGYEYPEGTALQPGQNIKVSINEYAW | | |
| | | KDNNGNAIEQVKPGRLLQYIARFEKTGAQG | | |
| | | ALPYWYPAGDLDWLVTHNNQVTGSYWLYYW | | |
| | | YGLMEGDLLKVDLSDENGKPQVLASYDASS | | |
| | | NQTQILLGGANEAEENTTLNLEALQDKYPN | | |
| | | GAHVTVYATDFTAPADMDNVAESVPAASDG | | |
| | | PYIVVDQDLAIADGHASLALNNLKGDSAYY | | |
| | | AVVTPATAQGAVAKDTVEAEYARRNGTATV | | |
| | | NYGNATGYSGTGYVSGTDEAASSDFFVNST | | |
| | | KDGYSEVTLRYSAPKVEGQSATRNVTLKIN | | |
| | | PSENTARNDVQDVTLNLPETKDANTWQTAK | | |
| | | IRVYLPLGLNQFTVEGFGAQGVLIDSLGIN | | |
| | | SADDSSVTRYEAEASSNTENGSANVSNNNN | | |
| | | ASNQKIVGNVGNGANNWFQFNNVTVPADGN | | |
| | | YTVTIGYSQWEYTDNNTWQIVNRWADMSVN | | |
| | | GGEAKHLVFANTRSWNNIWTTSVRVNLKKG | | |
| | | TNTLKFSNSDGSGSASKDGKPSGWAPNFDY | | |
| | | IQVAPTVDGGATYTTADGGEVKAITSLTAK | | |
| | | ADGLKDGVLTLTEGDSVDVDVNIDPIDSTD | | |
| | | PTLTWTSSDASVATVEDTENAGKAKTRSVK | | |
| | | LTDTRRIHALKAGETTITVVPTVNAADGVA | | |
| | | ATFKVVVNAEDTPTPDVDKSKLEAAVDAAN | | |
| | | KLDESAYTADSWKAFAEVLASAEGVLQNTD | | |
| | | ATQSDVDSAVELLADAQGKLVKADSEEGDN | | |
| | | PSGGETGDNPDDQTGDQDGDNAGSGEGDAS | | |
| | | TTDSDDKTDSGSHRMPSTGSAIAVVAAAAG | | |
| | | VLVLAAGGLLIVRHRRAKH | | |
| 15 | 15 | MNSMAQHTSHASINDPEVLPTPETVVLDAN | | |
| | | IFPSTWLTDLFLSLAEHTGLLEIIYSDTIL | | |
| | | EESRRAMIDDLGFAAHWVDRYLSSIQMGFP | | |
| | | YSRVVPDPDTMHRIVLPDPDDSHVVAAAVA | | |
| | | AGASTIVTYNLADFPESELAPYAVKALHPD | | |
| | | RVLTKILCNHTKPVADVLREIVSSKTRPPR | | |
| | | TMPEELEQLHRLGLHSFVEAAAPHVL | | |
| 16 | 16 | MTLTDSIPLAEVRPDSPYLAYGDGTALPLD | | |
| | | ADELRLIINALAEQDEMITTGEAAKILHVS | | |
| | | PRTVARIVDAGEIPSVRYGRLGNRMVSKRD | | |
| | | VLAFLDKSSQRTSEGLDSMRTAAYQGGLDE | | |
| | | LDGAAYIAREN | | |
| 17 | 17 | MANKTAANRKPSMGDVAEAVGVSKTTISRY | 35.8 | ['DNA-binding |
| | | LHGEFGCMSPETKARIEAVISELGYRPNKM | | transcriptional regulator, |
| | | AQGLKATVSHLVGVTIADIGNPFSSLLLKG | | LacI\VPurR family (PurR) |
| | | IQQECRARDVQLLVSDSNNQASFERANIES | | (PDB:1BDH)', |
| | | LLDAQVDGLIVNTVGNNDDWLSEYCARRNH | | 'Transcription', 'LacI family |
| | | KPMVMLDRIVQPIVCDCVATDNHGAVFEML | | transcriptional regulator, |
| | | DYLVDRGFDYVVLVTRPSDGISTRTMRREA | | kdg operon repressor'] |
| | | VEQYFLDRGLRGEVLVYREDAADLADCLIS | | |
| | | TIAAHGGERICLFANNDETMHDVLEALPAS | | |
| | | PDGHVGVCAFANERWAKYSGIGITCLDQNP | | |
| | | VEMGRAAARVLLARIYDGYDGPYELKEMPA | | |
| | | RLCAFASTEA | | |
| 18 | 18 | MDMSMDTHMDTSMDIHMDTSMDIHMEMPMD | | |
| | | IHMEMPIKSKVSKLDGSCT | | |
| 19 | 19 | MITWIMGILLIASFACFVIYAMRGGNLTIG | | ['Energy production and |
| | | FFILTIVWTLVYFLGIPFGVGEPFDVIKET | | conversion'] |
| | | FAEPALTYGPTIVQIVCGAWFGRVLVDTGI | | |
| | | AASISYRTAKVGEKNPVLATICVAFVTCLI | | |
| | | FTSAYGVGSAIAIGVILFPIMGRLGVPKRV | | |
| | | AVPVFTLSIGAAMWINSVMFVQFAAFYQGY | | |
| | | ESPDGQNIEWGTHYLSFGIPAMLVQMAAVV | | |
| | | IFILINAKAIENGTPYEVGDPNERPASVPE | | |
| | | VPIWTYVLPVVPVALSIVFQWDSVPALFLA | | |
| | | VIIAFFGTGYMKTYKGFVSILNSTAKTAIG | | |
| | | DIGSLIIMLLVLRFFQHAAVTVMADFGPAL | | |
| | | TAVVPNNEFILALAVCILAPLALFRGPLEL | | |
| | | YGAGSAVISILMGMGVENSWFLFAMLVVPS | | |
| | | MTCISSCVTQSWNMWAVEYNELEPKTFLKN | | |
| | | GVPVYWLCTFPIMGLASLLLF | | |
| 20 | 20 | MTTIGINTLVYMTELANGTPQSDLLPIIAS | | ['pfkB family carbohydrate |
| | | HGITLAEVRREYIASDTEFDLIAAAAQANG | | kinase', 'Glycolysis', |
| | | LDLFYSVPESLTIDGAVNPGFAGFLDEARR | | 'Sugar or nucleoside |
| | | MGVTNVKFNQGDVKDVAKSVIDDIDAAAAS | | kinase, ribokinase family |
| | | YGVTLTIENDQTPENGTLDCTVASLRHIKE | | (RbsK) (PDB:2QCV)', |
| | | LGGNIGYTFDLGNWFWRGENAGEAFAQLLP | | 'Carbohydrate transport |
| | | SITVFHLKNVNGAATREELATTMLEDGVID | | and metabolism', '2- |
| | | WKAMLPQLDASVPVFLEFPIPADGVAAQVA | | dehydro-3- |
| | | QVREVVGQKTPIMSEVMTIGEPMVNLIADS | | deoxygluconokinase |
| | | AETFMEARTLPREMAGAEFNVAIGVKRQGH | | [EC:2.7.1.45]'] |
| | | SISYVTTLGNDWQGDLIVDYMNNIDIDTTN | | |
| | | IRRVDGAATGYQLKVRSSDGEPKVIYFRAG | | |
| | | SAASQTAPDIVDGIDFDGLKILHVTGIFSA | | |
| | | LTDNTYATVMRLVDAAKAHGVTVTFDPNPR | | |
| | | PTLWNSEEQMIEATNRIAAKCDVFMPGLNE | | |
| | | GQLFSGLTDPRAIADYYLDMGVRQVVIKLG | | |
| | | AEGSALFERDESGARRETVVPSFEVDVVDT | | |
| | | VGAGDGFASGVITSLLEGLDDEHLLERANA | | |
| | | VGAIQVTSVSDSEGLPTAGELAEFIANTPR | | |
| | | KEVSL | | |
| 21 | 21 | MPGSQISFALYSVYLKSLLQNNNFGYTLIM | | |
| | | VLDINQRMLTTQETAQRLGVSTARVSALVK | | |
| | | NDDLQSIAVGKTRLITALSVAQHQHQDSRP | | |
| | | GRLFAPHIALGTLYLLSGVEATWLNAKERY | | |
| | | RIRNYLKTVKADRLARLCARRATTMDMWCP | | |
| | | SDAIPMLVSDIAISAATGELAVAFGLARTD | | |
| | | KVEGYIASDSLEYLVDHYDLETDLAPSTVR | | |
| | | LHISELIDKQTSSMPVGICAVDLTESEDIR | | |
| | | ERNAGLVMLEHLLTGFRSNSAREKVGNAIN | | |
| 22 | 22 | MQSINIQCISHPWSTVHDLSIADPTGNWLL | 47.1 | |
| | | AGGLMVQVHAILGGLPIRPTQDADLLMDLI | | |
| | | SQPNEANRVRHLLSSFGFTIHPGTLTGYTT | | |
| | | RMVSVNGSIVDVLVADHLPNHLAKESTYSG | | |
| | | FPILPMPGGAQAIERSMTVEIDSGTDRFPL | | |
| | | RVPDLLGATMLKSAAWETDKARDRSRHLSD | | |
| | | AALLLSLMREPQIELARLHSKTDRKRIRTL | | |
| | | TQHLGRDAEAWDFLNLDHRRYGLRALQILA | | |
| | | QY | | |
| 23 | 23 | MEKIVDTQEPISFLELLGFSDSTLQEYTIR | 53.9 | |
| | | LNQSNENWFDVTNTYYSNNQQLMDWVFTKT | | |
| | | WANDAKTKGKIPTNKVLQFIQLYKDEKPTI | | |
| | | HWLFIGGFEIIGESLQANGNTLYEYKTIPQ | | |
| | | FKTLSGRSVVKYRKYRGDTQLINDLRNNDR | | |
| | | RKRFISNLALDKITQSPISAQPFPGYLNIR | | |
| | | LSFPELAAAVKNDEWRSALNSINAVYLQTD | | |
| | | TLTGWHYVGSAYSRKGGTHGLLSRWEEYVS | | |
| | | GDHTGENKQLQQLVKSKGKEYIEDNFQYSI | | |
| | | LEIFDSRISIKDIIRREHWWMTTLSSVYDP | | |
| | | EGNPENCHGYNTKLEWNRTAEGKPADKA | | |
| 24 | 24 | MKLQVAIDRVDIPRVEAIIDQVAGEADIIE | | ['3-keto-L-gulonate-6- |
| | | IGTSLTKEYGLRALAPVCERLAARPAGETG | | phosphate decarboxylase |
| | | RKAVLLGDIKTCDEGKYEFDLGFDCGFAYL | | (UlaD) (PDB:3F4W)', |
| | | TVMGSSSLGTLEVCAASAADHGGEMMIDLL | | 'Carbohydrate transport |
| | | ECDESRIELISGFPDAVYCLHTSVDSGATA | | and metabolism', '3- |
| | | DPVGQVRAFKARFPQITRIGIAGGIKHDQL | | hexulose-6-phosphate |
| | | AGLAAEGVEIAIMGSAITKAGDIAAACRAC | | synthase [EC:4.1.2.43]'] |
| | | ADACHSA | | |
| 25 | 25 | MPSFVVGAYASLPQGREAQEAYYDLLGGQP | | ['Domain of unknown |
| | | WIDGTEIPFPGDLAETADRIWLAGQLPRHW | | function (DUF4862)'] |
| | | KNNTVTAIPGTMQHVWKDPNFGLASPDEDG | | |
| | | RRFALAFFKQLRDALADFAQWRGSQDVKFV | | |
| | | EIHTAPTRIASRDAMTASLQALAQLDWSGA | | |
| | | KLVIEHCDAYVEGRKPEKGFLPIEDEIALC | | |
| | | RESGIGLTVNWGRSVVEGRKVQTAVEHIEA | | |
| | | AAGAGVLAGLMFSGSGPEETQYGYGWIDGH | | |
| | | LPMNPDEPTSLMDAAAIGVAVKAAGEQGEP | | |
| | | LAYLGAKVCVPKDATAEERLGYLARIHDAV | | |
| | | LAGRAGA | | |
| 26 | 26 | MSGVAAGAATYLAFDIGGTKIASGFVTLPD | | ['ROK family', 'Sugar |
| | | ENSAHADCGRKPRVEAQCEIPTEASRGGDD | | kinase of the |
| | | IRERLTAFASRQLARARDEGAVIRGIGIAA | | NBDIVHSP70 family, may |
| | | AGVPDSRTGVIVSATDILPGWRGQRIYDAF | | contain an N-terminal |
| | | AKVTDLPVHMVGDVGAHGLGEAGYGAGRGH | | HTH domain (NagC) |
| | | GIVLSIGVGTGIGGAIVVDGTLFSGAHGVA | | (PDB:1WOQ)', |
| | | GHAGHVPSGLGRGFLCSCGTREGHIEPVAS | | 'Carbohydrate transport |
| | | GTGLKDLYNARCDAEEGEPVADGSQVAARA | | and |
| | | AAGEPLAVGVIEDSARALGECIGGMGNLID | | metabolism!!!Transcription', |
| | | PDVIVVSGSVVKAGPLWWNALRAGFEDSAL | | 'glucokinase |
| | | QLVRSAPLVEGELGGAAPLIGAAVAVRRHV | | [EC:2.7.1.2]'] |
| | | AQGRP | | |
| 27 | 27 | MHQVIEKIRGGLVVSCQAYPGEPLRHPETM | | ['Putative N- |
| | | AQMAMAAVEGGAVGIRCQGLADIAAIKGQV | | acetylmannosamine-6- |
| | | KVPVIGIWKEGDEGVYITPTLRHARCCAAA | | phosphate epimerase', |
| | | GADIVAIDATGRPRPDGLSYADTVHALHDE | | 'Putative N- |
| | | GVITMADCGSFADAERAVEAGTDIISTTLS | | acetylmannosamine-6- |
| | | GYTGERLKTDGPDFELLERMVKAFPDMPVL | | phosphate epimerase |
| | | CEGRIHTPDQLHRVMECGAWAAVVGTAITH | | (NanE) (PDB:1Y0E)', |
| | | PTTITRWFAAKL | | 'Carbohydrate transport |
| | | | | and metabolism', 'N- |
| | | | | acylglucosamine-6- |
| | | | | phosphate 2-epimerase |
| | | | | [EC:5.1.3.9]'] |
| 28 | 28 | MIKRNGKFKAAVAAAVASLMLLSGCGGGTQ | 28.7 | ['Bacterial extracellular |
| | | TAAKTGTAVADTITAQVAYASRDFSPSTTS | | solute-binding proteins, |
| | | GALPMAANWHVTEPLYALDYSTYEPYAALA | | family 5 Middle', 'ABC- |
| | | KGDPEKVSDTEYVVTLRDGAKFSDGTAVTA | | type transport system, |
| | | NDVVSSYQRTTATGSLYISMLDFIDSVEAK | | periplasmic component |
| | | SDTQVTFKLKKAFPLFKQRLALIQIVPSSM | | (DdpA) (PDB:3RQT)', |
| | | SDADLKDKPIGSGPWKYAEITDQQVKFERN | | 'Amino acid transport and |
| | | DLYNGSYPAQAKNMVWNVTVDDTARVTAMQ | | metabolism', |
| | | GGKTDIMEMVPAQALQTLQSSGSELKTAQG | | peptide\Vnickel transport |
| | | FNLPFLMENTKKKPFDDKRVRQAVFYAIDV | | system substrate-binding |
| | | DNLISNQMSGQAEAATSFLPKDSQYYHQAK | | protein'] |
| | | NVYTKDTAKAKELLAEAGVTTPISFTLYTT | | |
| | | DHSWITQLAPQIKNDLAEIGMNVDIQSMKS | | |
| | | SALYPSITDKDDADYSMVLAPGDPSVEGND | | |
| | | PDLLMNWWYGDNAWTKQRSFWKGSDGYNQL | | |
| | | HELMDKATAASSDSERQKYWNQCFDLLSEE | | |
| | | LPLYPLFHRKTTTAVRKGAFSSWEAIGSTG | | |
| | | INLVKAKLN | | |
| 29 | 29 | MALGVTFLVFFLMSFSQYDPAVAALGENST | | ['ABC-type |
| | | PEALAAFRHEMGYDLPW | | dipeptide\Voligopeptide\ |
| | | | | Vnickel transport system, |
| | | | | permease component |
| | | | | (DppB)', 'Amino acid |
| | | | | transport and |
| | | | | metabolism!!!Inorganic ion |
| | | | | transport and |
| | | | | metabolism'] |
| 30 | 30 | MGVYGVNKDSVAARVAQAFPVTLQLTFIGL | 38.6 | ['Binding-protein- |
| | | IIAIVFAIVFGVLAALYRDTWVDQIIRVVS | | dependent transport |
| | | IIAIATPSFWLGVLLIYVLQIKMAWLPGSG | | system inner membrane |
| | | DLVPFTQDPGAYLARMAMPSFALGLPVAGQ | | component', 'ABC-type |
| | | LTRIIRTSMVEELDKDYVRTAIGAGVPKSV | | dipeptide\Voligopeptide\ |
| | | VVSRNVFRNALITPVTTLGMKIGYLMGGAI | | Vnickel transport system, |
| | | VIEVIFALPGMGTAMFDGINGNQPMLVQGV | | permease component |
| | | VLVVALAFIIINIIVDLLYVLINPRIRTV | | (DppB)', 'Amino acid |
| | | | | transport and |
| | | | | metabolism!!!Inorganic ion |
| | | | | transport and |
| | | | | (metabolism', |
| | | | | peptide\Vnickel transport |
| | | | | system permease protein'] |
| 31 | 31 | MLFGKNKAAEAASRPGVKFNRFSKMTVGSK | 44.1 | ['ABC transporter', 'ABC- |
| | | ISFVVIALLIVCAVFAPVLSPHDPLEITMS | | type |
| | | YQAPTGEHWEGTDNLGRDVLSRVLYGARYS | | dipeptide\Voligopeptide\ |
| | | LVIGLSSIVFALIVGSLIGALAAVTRTWIS | | Vnickel transport system, |
| | | ELIMRVIDVFMSVPGIALAAVFVSILGQSM | | ATPase component |
| | | IGIIISIGVLYVPQIARIVRANIISEYGKD | | (DppD) |
| | | YVRAVIVSGARAPWILFKHVTRNIAAPVMV | | (PDB:4FWI)!!!ABC-type |
| | | FTTLSVADAIVFEASLSFINAGIPEPTPTW | | dipeptide\Voligopeptide\ |
| | | GNILSSAKAGVIFGYWWQAMFPGLAIMITV | | Vnickel transport system, |
| | | LCLNILSEGITDAMVAAPTAPVTKSAADAE | | permease component |
| | | AERREDRLLTDPVAAYREQAESLADSLAAL | | (DppC)', 'Amino acid |
| | | KEAELKRTDRFEPTSTAAPVIEVKNLCIKF | | transport and |
| | | PRHGDVNVVDHVSFAVRPGETMGLVGESGC | | metabolism!!!Inorganic ion |
| | | GKSITSLAIMGLLDPKAEISGEILFGGRNL | | transport and |
| | | VGMSPKEHNALRGHEIAMVYQDALSSLNPS | | (metabolism!!!Amino acid |
| | | MLIKSQMKQLTSRGGTRSAEELLELVGLDP | | transport and |
| | | KRTLESYPHELSGGQRQRVLIAMALTRDPK | | metabolism!!!Inorganic ion |
| | | LIIADEPTTALDVTVQKQVIDLLNELREKL | | transport and |
| | | GFAMIFVSHDLALVAKVAHSITVMYAGQVV | | metabolism', |
| | | EQGSTKEILTDPRHEYTRGLLGSVTSIEAG | | peptide\Vnickel transport |
| | | AKRLHQVPGTVPSPADFPKGDRFAPRSSHP | | system ATP-binding |
| | | DVGLNTRPIFERVPGTHHYYAALPADADVT | | protein'] |
| | | PAAATVSTQEGGAR | | |
| 32 | 32 | MTNETTAPSGTESRILDQIVAEITGVIAKM | | ['SIS domain', 'D- |
| | | DEGDIERAMPLIGKTGRVYATGEGRSGFQA | | arabinose 5-phosphate |
| | | RSFAMRMMHIGYTSYMMGETICPSMHEGDV | | isomerase GutQ (GutQ) |
| | | LLAISGSGATRRTVEDAEAAKKLGVKVIAV | | (PDB:5UQI)', |
| | | TSKPESPLAAAADAVIVVPGRVKGEAGGSI | | 'Carbohydrate transport |
| | | QLLSSLFDQSVHIALDALCLMLSRRDNVSD | | and metabolism!!!Cell |
| | | ADANANHANVGL | | wall\Vmembrane\Venvelope |
| | | | | biogenesis', '6-phospho- |
| | | | | 3-hexuloisomerase |
| | | | | [EC:5.3.1.27]'] |
| 33 | 33 | MSHAAETPRKDPNVPIIELRDVEVVFTTRA | 48.5 | ['ABC transporter', 'ABC- |
| | | GSGLFHKNRITAVNKVNLKLMPGQTIGIVG | | type glutathione transport |
| | | ESGCGKSTTANVMCGLQQATSGKVLFKGQD | | system ATPase |
| | | VTHRTAKERMDIGRVVSVVFQDPATALNAR | | component, contains |
| | | MSVIDQLLDPLVVHKLGSKEERDRRAHELI | | duplicated ATPase |
| | | RMVGLPTSVLGALPGQLSGGQRQRVAIARA | | domain (GsiA)', |
| | | LSLKPDAIIADEPTSALDVSVRAQILNLLS | | Posttranslational |
| | | DLKRTLGLSMVFISHDIQTVRYISDEVMVM | | modification, protein |
| | | NHGTVVERGKTMDVMRNPQDGYTRILMDAA | | turnover, chaperones'] |
| | | PSLLHPTAAECA | | |
| 34 | 34 | MATQFRGVIPPVVTPLTASGEVDKASFARS | | ['Dihydrodipicolinate |
| | | INRMIDAGVDGLFTLGSSGEVAFSTDARRR | | synthetase family', 'Lysine |
| | | EIIQTVIQVVDGRVPVFVGCIDTETNRVIE | | biosynthesis', '4-hydroxy- |
| | | HAKEAKELGASAIVATCPFYALGGMAEVER | | tetrahydrodipicolinate |
| | | HFRLIHAAVPDLPLFAYDIPVCVHTKLPGD | | synthase\VN- |
| | | LLVKLGRDGVLAGVKDSSNDDVAFRFLVDD | | acetylneuraminate lyase |
| | | NAKAGHPLTLLTGQEVVVDGAYMAGADGSV | | (DapA) (PDB:2R91) |
| | | PGLANVEATGYVRMWKAAEAGDWATVRKEQ | | (PUBMED:27574185)', |
| | | DWLAALMRIVTVPQGVAGFGSGVGAFKTAM | | Amino acid transport and |
| | | ALLGVFDTNQMPDPVLPLKGENVKRIATVL | | metabolism!!!Cell |
| | | EECGMKLERTPEEVSASTEA | | wall\Vmembrane\Venvelope |
| | | | | biogenesis', '4-hydroxy- |
| | | | | tetrahydrodipicolinate |
| | | | | synthase [EC:4.3.3.7]'] |
| 35 | 35 | MNGTADRATAGSRATDTEPDAPQGQYSTQE | | ['Bacterial regulatory |
| | | LAALARLSLGNWSTTQVSSRSRCDETMDAI | | proteins, gntR family', |
| | | KSYILRERLQPGDVLPTETQLCDTIGASRS | | DNA-binding |
| | | SVREAVRKLEALNIVKVEHGKGTFVGSLSL | | transcriptional regulator, |
| | | DPMVETLAFRSMASVGKNFTDLQDVVELRR | | FadR family (FadR) |
| | | FLDLGCAEEVCASLAGTEQPRLTELAERMS | | (PDB:1E2X)', |
| | | AEAKEGKTFTGLDIEFHRGILDSLNNTVAK | | 'Transcription', 'GntR |
| | | QMVRSLWLVHMAVLPQLGLAASSELDRTAD | | family transcriptional |
| | | AHHRMLNAALAGNVDDYREAVEDHYEPIES | | regulator, transcriptional |
| | | ILKRRIVQEQ | | repressor for pyruvate |
| | | | | dehydrogenase complex'] |
| 36 | 36 | MAPWQTTDFPHGAGTVIAEFSVRADGPIWR | | ['BNR repeat-like domain', |
| | | AQVPATADGIPTAAPYLCLAVRDGHLTLTA | | 'Neuraminidase |
| | | RSLSPDPADPNAQSHVSLDIEDAFGLDPGT | | (sialidase) NanH, contains |
| | | IHEAALTFGAFGTRIYLDGYQCFACAGNLN | | C-terminal autotransporter |
| | | PSRVHPSGAFDLGSPNAIACNAFLVDPVDW | | domain (NanH) |
| | | DAVRIAEHAAAAKPDIVFASDRLSPRDTDR | | (PDB:1EUT)', |
| | | IALADAGSVHARFRLRGRGQHGTILAAGVD | | Carbohydrate transport |
| | | GSERMTVAIGAGGLTLAMRDESGAGIACHA | | and metabolism!!!Cell |
| | | PGHWDDGGWHDLAIRSSRGAVDLFMDGVSV | | wall\membrane\Venvelope |
| | | LHQPGQMWFADLAGPRHGRKGIDAFTVGRN | | biogenesis', 'sialidase-1 |
| | | IAGVRLMGEVSRGGLYVHALTDGQIARLAH | | [EC:3.2.1.18]'] |
| | | TPPMVTTALFDAGYAGSASYRIPSLIRTVR | | |
| | | GTLIAGADQRTAISNDAPNHINFVIRRSTD | | |
| | | GGRNWMPMQTVIDMPGREDGLDGASAIDSC | | |
| | | SVVDRSIGRITVLIDLNPGGIGLTNCERGI | | |
| | | GVNRDGVLRLRDAQGNETTLDAVADAGDVW | | |
| | | RSPMHADPSQRWHAVPTCYIAQIHSDDDGE | | |
| | | TWSPPFLIDAMVKEEWMHFMGVCPGTGIQL | | |
| | | DKGPYAGRLLMPFYCSGQSRTHYSGGALIS | | |
| | | DDGGETWRCGRMINEGREINGTVVDPATMR | | |
| | | DDDATTSETTFVQRADGDVVAFFRNQHASG | | |
| | | RVGKAVSHDGGDTWDELEFDPALPEIFSQP | | |
| | | NALAVPQLGTDAVLFANASQMMPYRGRGMV | | |
| | | RLSEDGGRTWTGSLCVHPHHHVYQCMAICE | | |
| | | TTHDVECEGSQLGLLWEIETTGVYITHIPL | | |
| | | AWFSHGHDKGVAANHTDDKESS | | |
| 37 | 37 | MSSRAPRPARNLRYFHAIRHRHRLKQLPCQ | | |
| | | SRMAIKRLNTRHTFWPITPVTPPYTTKYGK | | |
| | | RTEKLLTFVRSLVATPAGLEPVTSAVTGRR | | |
| | | SNQLSYGAICTSMKHKRIYYSITGPRRAAR | | |
| | | RVAPRQCQAHPLPLQAIATPAIPALPIPWW | | |
| | | AVVWRGFDRYADRAAQA | | |
| 38 | 38 | MKPRRRRIAAMVTSLAAALVMSLSCVTTAS | | ['General function |
| | | ASSVYLSVPIYVQEQSNWCWAATSKSVSVY | | prediction only'] |
| | | LGGSNSSQCQYVKWGKNSSSCANVTGDLST | | |
| | | DVRRALSSAGIRNTGSMINSAASTAIVSGQ | | |
| | | INNSKPLMVRWGWDSGGGHMLVIRGYTSDP | | |
| | | GYLVVSYIDPLQSYYSSGTYDWMKSGSGHT | | |
| | | WTHTRYGFSR | | |
| 39 | 39 | MSTFYIVRKSLIQALRILFAAMLFVVAIGT | | |
| | | VCPDIARAENLPESTQINEFAQSDEARTQT | | |
| | | LALMEAVQNDGSPEASTQIAIGYADKVHYL | | |
| | | RYDDSGAITNTITDPQDYDWVAPVEVEGRL | | |
| | | VGRITIWDNNGSLEVGNFSPDIEEASLLDD | | |
| | | DDSTTLISDGFSRAYYSMENSRISPLNEAA | | |
| | | RAIVPESVQVEQGDIAIRKNAPSGEDDSAG | | |
| | | GGSIGVVSSDTATGTDSSMNIFMLMAVFIV | | |
| | | VTAVLALMCRILWRGPNRRNDR | | |
| 40 | 40 | MRKRQPLFSGLCLAVFALMSGCTGAPVQSD | | |
| | | DAGAHLEFRAYAESGTVRIESQSETVLETR | | |
| | | IKGDWLKVAPDDWLENDRPLTFTVERDSGG | | |
| | | VEDNVSCEIIFSGHTIDERRVTGPETSATC | | |
| | | QYDTWRDAIDY | | |
| 41 | 41 | MADSPASDIKPKAYRPRIVDEWQLAPAIWD | 49.2 | ['Domain of unknown |
| | | EVRHRVDDDSANKGQWILTGSSTPLNENRP | | function (DUF4143)', |
| | | NHSGAGRIGRIRMNPLTLYESGLSTGAVSL | | 'Predicted ATPase, AAA+ |
| | | SGLFEHKFAAAKSEISTQMLLDAVCRGGWP | | superfamily', 'General |
| | | EAVALPVSDAQILIREYMRLTLTESVPRQG | | function prediction only', |
| | | KDPDVARRLLDSLARNISQAVTFKTLRKDM | | 'uncharacterized protein'] |
| | | YGTEENLDDFISERTVSGYTAMFENMFVID | | |
| | | PIKGWVPPARDPKRLQTKARRYFADPSIAA | | |
| | | AMLGMSPAALIGDWQTFGFLFENLCIRDLL | | |
| | | VYARSLLDIGIEPVRYYRDDSGLECDAIIE | | |
| | | LSDGRWGGIEIKSSEDKVPEASANLCRLKD | | |
| | | KLLRNPSARTREPEFLAVLVGVGEFAYQRD | | |
| | | DGVYVIPVGVLGA | | |
| 42 | 42 | MKGSMMSVRYGAKRVIASSMVFSVLCALAA | | ['Bacterial extracellular |
| | | CGGGSANSQAGSVSVACSQQEDFCQAMTAA | | solute-binding protein', |
| | | FQKETGIKTTYVRLGAGEVLARLETASGEF | | 'ABC-type Fe3+ transport |
| | | DVWAGGQAENHLLADDKGWVEKYVSPNASD | | system, periplasmic |
| | | LPDEYNDDNGIWSGFYTDSIAFCSAASELE | | component (AfuA) |
| | | KKGLEAPTSWEDLLDPALKGSVAMPHPATA | | (PDB:6IVY)', 'Inorganic |
| | | GVGYMAMYALAALNNGDEDAAISYFKQLNA | | ion transport and |
| | | NVMQYSKSAATGTEQAGRGEVAVAIALDSD | | metabolism', 'iron(III) |
| | | CQKAIAAGYSDLKTTYPKEGTGYEVGAISV | | transport system |
| | | LKDAKNADNAKKFMDWILTADAQNLYADVP | | substrate-binding protein'] |
| | | SYAAPTNPKATVGADVPRQDIVKKVAWDTR | | |
| | | KAADGREAFIAAFESDVASADSAQ | | |
| 43 | 43 | MISPTYEFPENFIWGAATAPHQIEGNNTAS | 38.0 | ['Glycosyl hydrolase |
| | | DWWAREHSPRTDVSEPSGDAADSYNRYRED | | family 1', 'Beta- |
| | | IRLLADSGLTMYRFGIEWARIEPVEGRESK | | glucosidase\V6-phospho- |
| | | AELLHYRAMIDACREFGVEPMVTIYHFTMP | | beta-glucosidase\Vbeta- |
| | | LWFAAEGGWKRPDALEKFERYVRYVLPILN | | galactosidase (BglB) |
| | | DVTWICTINEPNMVALTQGGTEGTDFVAAS | | (PDB:4HZ6)', |
| | | LPAPDPVISKTLVDAHHMSRAVIKSELPDA | | 'Carbohydrate transport |
| | | KVGWTIACQAFHAVPGCEKEMEEYQYPRED | | and metabolism'] |
| | | YFTEAGAGDDFIGVQAYLRTFIGKDGPVPV | | |
| | | DDDVERTLTGWEYFPPALGIAVRHTWDVAK | | |
| | | HTPIFVTENGIATADDRRRIDYTFDALAGL | | |
| | | HDAMDGGIDVRGYTHWSLLDNYEWGSFKPT | | |
| | | FGLIGWDKDTFERHPKASLNWLGSISRTGV | | |
| | | VTHPYR | | |
| 44 | 44 | MQSAGRPAPAQDNKLPLWQRFIKGRGIAYI | 36.9 | ['Binding-protein- |
| | | VLAVIGVVWIFPFLWMVLGSLKTQREILAK | | dependent transport |
| | | PPKLMPEHATLANFSQWFTQLNFGSYFTNS | | system inner membrane |
| | | LIVAVITVLGNMVFCSMVGYALAKMKFVGK | | component', 'ABC-type |
| | | NILFGAVMVTLMVPSVATFVPLFVIISNVH | | glycerol-3-phosphate |
| | | LANTYAALILPFLCQPIGVFLMRQFIGGIP | | transport system, |
| | | DALMEAARVDGAGELRIFFQIILPQCGPAL | | permease component |
| | | ATLSILTFLSSWNNFLWPLVSAQSEEMYTL | | (UgpE) (PDB:1VR4)', |
| | | PVALSLYSTGQNATNYSVLLAGAVLVITPI | | 'Carbohydrate transport |
| | | LLLFVFLQRYFIQGVAMTGIK | | and metabolism', 'multiple |
| | | | | sugar transport system |
| | | | | permease protein'] |
| 45 | 45 | MAKRTKAQAKGAGSSLCRRQTLLAWGFALP | 53.8 | ['Binding-protein- |
| | | FAVIFCVEMLIPLISSMAMSFTDITSRDLR | | dependent transport |
| | | TPFNVNFVGLDQYIALFGDKRFLHSLGVTG | | system inner membrane |
| | | IFVLIGLPITMIIALAFAVALNKGSQHLNA | | component', 'ABC-type |
| | | FFRALFYAPVVASVVAVSVVWRYILQADGL | | sugar transport system, |
| | | LNSLLSLVGVQGPDWLHDTRYALPALMIMT | | permease component |
| | | IWRNMGTLMIIFLAGLQAIPEELKEAAAID | | (UgpA) (PDB:3FH6)', |
| | | GASKWRTFRSITLPLMKPTLLLGAVLLSVG | | 'Carbohydrate transport |
| | | YLQFFEESFVMTQGGPLDSTLSAAYYVYQK | | and metabolism', 'multiple |
| | | FGFGQYGIASAASWVLFIIIALVSVLQFRI | | sugar transport system |
| | | LRSED | | permease protein'] |
| 46 | 46 | MKRSMFKMATAIIASAAMLTSVAACGRTSA | 26.4 | ['Bacterial extracellular |
| | | TSDSADDVTTIDSGKATGDLTIWAMGNEGD | | solute-binding protein', |
| | | LLGDFVKDFEKENPDVTVKVTAIPWSSARD | | 'ABC-type glycerol-3- |
| | | KIQTAIAAGNGPDVAQMGNTWMADESNSES | | phosphate transport |
| | | TVPSNFDMSGFFEGPADNYKVGDQQLGVPW | | system, periplasmic |
| | | YVDTRVLYYRTDIAEKAGITEAPKTWNELK | | component (UgpB) |
| | | TMAEAMQKVDGVDYGMRIGASGTDCFIGFL | | (PDB:2Z8D)', |
| | | PYAYSAGAALSDDGQTKWTIDSDAMAEALD | | 'Carbohydrate transport |
| | | YVTGFYKDGIADTNADVSAGADIADFVAGT | | and metabolism', 'multiple |
| | | TPMMLQGPTAVSQVEELGGDDIKGKYATVT | | sugar transport system |
| | | LPAMDDSSDMGTSYLGGSGLVTFKDSKNKQ | | substrate-binding protein'] |
| | | AAWKFIQWTSQPDVQAKWYTLSSDLPAAQS | | |
| | | AWDDDALTSSTTLTAFGDQFEHAQGVPAFT | | |
| | | TWAQVSSAADRTFEQIAKGQVSVADGLKSL | | |
| | | QSEADSIGIGE | | |
| 47 | 47 | MRRATVYDVAKKAGVSTATVSFTFRRPDKV | 28.6 | ['Periplasmic binding |
| | | KPSTRAKVLRAAKDLDYVPSANARGLARGN | | protein-like domain', |
| | | TGVLGLYSFDMLIERPLGDEDDEDSYSGGN | | 'DNA-binding |
| | | DVVSENGKPIFSNREGGSFDCPSVLSYPLY | | transcriptional regulator, |
| | | VDEVQRGFELECRRRNRAVLLGTAIRHDDG | | LacI\PurR family (PurR) |
| | | TGITDVAGRVDGLAVFPNESTNTMPLEALC | | (PDB:1BDH)', |
| | | RSIPIVRLSEGDGDEPAAYISCDNETGMNQ | | 'Transcription', 'LacI family |
| | | LIDHLVDVHGVHDMEFVGSLDNYDSRHRFA | | transcriptional regulator'] |
| | | AMRAKLKMKGLRVPDLPLDDSMSGTHEWFV | | |
| | | DLCEVIDAGRLPQALLCATDQTAFEVMSIL | | |
| | | RDADVRVPQDVILTGFDGVLAGQVLTPTLT | | |
| | | TVRQPLELLGRLAARLLDEQAGEPWGKPER | | |
| | | FRLPVKLIVRGSCGC | | |
| 48 | 48 | MEPYSAIQVTADMTNAHTQKREITALEQTM | | ['General function |
| | | HRMSDVTGTIITLREEGTIPTDAGDINVIP | | prediction only'] |
| | | AWKWALQSKN | | |
| 49 | 49 | MTSLSKPAMFNLEHILTGMWRSGKTFQLFQ | | ['Predicted ATPase, AAA+ |
| | | LINDLMQSGVPRERMFYENFSDERLQPMPE | | superfamily', 'General |
| | | DMLDQVITEFWRQDPSSRTQGA | | function prediction only'] |
| 50 | 50 | MIPRTIAQELTPMLSWFPVVSVTGPRQSGK | 55.9 | ['Domain of unknown |
| | | STLIKNMLPDYEYVNLEDETTRLSAIEDPV | | function (DUF4143)', |
| | | GFIHAHERKLIIDEAQYAPSLFSQIQVMAD | | 'Predicted ATPase, AAA+ |
| | | ERGTMGQYMLSGSQNFLMEKRIGQSLAGRV | | superfamily', 'General |
| | | GMLQLLPLSYEEALQAKTDLTVDEFMFHGG | | function prediction only', |
| | | YPHLYDVPTPTDIYFRNYTATYVSRDVAEY | | 'uncharacterized protein'] |
| | | LDVRNLTDENTFLRLCAENAGNLLNLTALA | | |
| | | RDANVSFNTAKEWLSILEASFIVFRLVPYS | | |
| | | ANTRKRLIKTPKLYFYDNGLLNYLLGIHSP | | |
| | | QELADDPKRGDIFENLIISETVKRYRNRNK | | |
| | | DCELCFYRDTNQREIDLIDTTQRRNPTLIE | | |
| | | IKSGMTARPDFFKHLATIGEELGVPTDHRI | | |
| | | VVYRGTESFTSKNGRTITAKDYLCLAHS | | |
| 51 | 51 | MTVISSRSKIMGGNGRRGPRKSPSVWVTLV | | ['Binding-protein- |
| | | GVIAVLMLVLGLPVYKLFVAALSEEGRSAM | | dependent transport |
| | | VGAFSNGGETLINSIVLGLLVGVLGTFIGF | | system inner membrane |
| | | VCAYAETFIQFPGRKALHWLTLLPTISPPF | | component', 'ABC-type |
| | | AASTAIITLFGKRGMITNGLFGLEVNIYGL | | Fe3+ transport system, |
| | | SGLVMVLTMTFAPVAYLNIKGMFENIDPSL | | permease component |
| | | FEAASSLGASQLRTLIRVTIPMVMPAMLSS | | (FbpB)', 'Inorganic ion |
| | | FLVLFVEGIADLANPLVIGGDYRVLASQIY | | transport and |
| | | FAVAGSGNIAGAAGVAIVLLVPALSVELVQ | | metabolism', 'iron(III) |
| | | KYWASKKSVVTVTGKPTGSLKPVTSKAVVV | | transport system |
| | | PIMTVVTLWTLFVVSIYVTLFIGGFVKILG | | permease protein'] |
| | | VDNTFTWEHFRFVRRLGSDAIITTLTMTLI | | |
| | | AAPLAALLALAIGWLVVRHLPRFGKILDLW | | |
| | | GMLGVAIPGTVLGLGFALAYSQPTVLFGVN | | |
| | | ILPALAGGLAVGNGAIAIIMVFVARGNPTG | | |
| | | QQAFISAFKQINPQVEEAATSLGANPLTVV | | |
| | | RKVTLPLMSSAVVTAITYGITKSMTTITAI | | |
| | | IFITTPQTKVMTSQILDEVDAGREGNAFAY | | |
| | | SSLLIVLVLIVLGIANIFLTRLNQSKR | | |
| 52 | 52 | MRYAFVLTHLSPYIFRTVHSRAYTHLYTDI | | |
| | | YGTNEN | | |
| 53 | 53 | MHDEVELTRGYLLHRLSHEDERIRTLLTAH | | |
| | | TTNKHYIEGPQIFTLSILNMLGVSGKTNER | | |
| | | RIDYFRCRNMVYIFHPAFELRRNHMMKLNP | | |
| | | FVVSQRLAREATTEHIFHNVQ | | |
| 54 | 54 | MTISISDNDRQESSFFGKLDGLHSPTFSAT | | |
| | | SLKPMHGNNDSLRRVGNTIPRVNLYPRVMV | | |
| | | LII | | |
| 55 | 55 | MFPDIFIRSIIWRKQDIPYIAVNKLLIPSK | | |
| | | IVRRKHGLRIHRGSRIELAEKLGTIRTTTI | | |
| | | ERRSVRKTRILPLLTHEYLIVQGTPRLNQR | | |
| | | MIVNRSDD | | |
| 56 | 56 | MAKRSVKQRNLNIEVLRILAMFLIVACHAT | 47.4 | ['Acyltransferase family', |
| | | LHLPWLLHVDSNLDFLPGWKSALAYLVVQY | | 'Cell |
| | | GQVGVSIFFIISGYFLVRKTFTWQRIFKTW | | wall\membrane\Venvelope |
| | | FQMFCYSFISLIAVLIIARFTTLPNSIAPL | | biogenesis'] |
| | | LSGDDLWRTVLWSIVPFIYGSYWFITAYVC | | |
| | | LLLLAPFINCLFKHLSRRNMAALIIVLSFF | | |
| | | SIWILLGGRTTPWNNVVYAVLGYIMGGWIR | | |
| | | LYWYEVNDKIKSSYLWGIIVLSTIVMTVEN | | |
| | | HYAANRTWLATFLGWHEQIKPGIQIFPMII | | |
| | | GSSIFILFTKLDMTSIKGFGHKVVLKTASA | | |
| | | TFGVYLIHENMFWYRLIWPTIAAIFPTPNS | | |
| | | FISTVSVAFAIVLAVFISLSLIGFIADTII | | |
| | | VHPLTKIILKGWDTPHHTN | | |
| 57 | 57 | MEGKPVEQETLVHTQKSLNKPLIVISLVTG | 33.5 | ['Family of unknown |
| | | FLIGFGNAAAATAEFPVGSVLMWAQCVIFS | | function (DUF6020)', 'Cell |
| | | VLVYIFFSLTERLLSYCRHNDCQKWQKRMG | | wall\Vmembrane\Venvelope |
| | | RIDFSFSPVSLTIVASFIFLMWLPYLVILR | | biogenesis'] |
| | | PGVIEWDAGDQIAQGLGYSAFGQEPGQIYD | | |
| | | HHPFLEAIIFAQFIKVSIAITGSYKLGAFV | | |
| | | LVTLQCIGMAVAFSCLIAYIRENLKASFSI | | |
| | | ALSSTLFVALFPVFPFYFSTVLKDSFHALF | | |
| | | LLPWAIMYVEMVRTRLNCVAKASFSVTFII | | |
| | | FSVLVCLTRKTGPALVFLALIMLVFVKTSV | | |
| | | WKKLAAVVTAFVIFLSMSSFLPRYVYPALN | | |
| | | VVPTDSEQYYIIPLQMTARWGKDHPGEATE | | |
| | | KEKNIVSQFNIFTYDEMTKNYEPFLTDKAS | | |
| | | MYKLGDASLRNDYFKVWLSQGLKHPKSYID | | |
| | | AFAALESGWFAISKSPTGQPVYPYDTVGNQ | | |
| | | MTVFYKTVTNPDTTSEFINSPNDSMNDSMG | | |
| | | RWFNYFKQIPVINITTYTAFWTWLLPMFAV | | |
| | | YMMIRRNRVSLLLLQAVPFLLGIASLYASS | | |
| | | TAYISRYMLFAMYLAPLLIGIISSDQE | | |
| 58 | 58 | MRRRPGSANPMACSGPGNATADGSSKISAV | | |
| | | RFRAESSCGRFDDTAAVPSGLSASPDYRSN | | |
| | | PHGGTMTRRTSMRKAPADPLPENANRRHAP | | |
| | | LKRRRRWFRMVGGPDRPILLDPPPGDRRTP | | |
| | | VAAHDGCRPMPIQGVGRILQKTGSIVRPNM | | |
| | | RISVRRRGYLA | | |
| 59 | 59 | MMDFEPIAIIGRGCILPPHSCSPEELWDAI | | ['Beta-ketoacyl synthase, |
| | | VEGRSGIHPPMPERWGTRVDYVDPDRNAVD | | N-terminal domain', 'Acyl |
| | | RTYCGIGGFVTDYDQDERAASSERMRLNRT | | transferase domain in |
| | | QRMIVDSASQAIGEAGIDRSERARCRLFVG | | polyketide synthase |
| | | NMLADEAFGDQSLSEISGDVLDECVNEFGA | | (PKS) enzymes (PksD) |
| | | DARDAARRAIDDVILSRNDASETANAPSDL | | (PDB:6IYO)', 'Secondary |
| | | ARIPAEVLGMPDDPIVIDGACASGLLIVDL | | metabolites biosynthesis, |
| | | AARYLHTRAKPLAMAVGAMANMSITGNVSF | | transport and catabolism'] |
| | | AKIGGLSDKPARPLDANANGLVPAEGAAAV | | |
| | | LLCTLSYARLHGYPISGVIIGSYTTSDGHG | | |
| | | KAIYAPNPEGQRCAMRGALAQAGIDPDGID | | |
| | | YIETHATGTPAGDNSELTAIIGMLSERRNG | | |
| | | PVSIGSIKNLIGHGFPTAGTSNLLSVLESF | | |
| | | RHERYLPTHGVTTPHPLIAKHPELLQLHDA | | |
| | | PDPWPEPGNRPRRALINAFGFGGINSSVIV | | |
| | | EQYDDGRPDGPAHADAEPRDATYLAVQCVA | | |
| | | QANCEVPRTLLEDPMTADWRVFHTPPVLLP | | |
| | | HMDIAQRLAVLAAGNLQPMMAEPDRKETIG | | |
| | | AFLGQPSGLAVGARRELRIRLPEILDAITH | | |
| | | AEIPDERRRELRRWFADRVTTSIGATVEAA | | |
| | | LPGYMDNIVSGRIANMFDYRGPNCVIDGGR | | |
| | | FSFARSIEMASLVLAEHEADGTIVGSSFAN | | |
| | | PSHLVDQEAARLATATLIMLRPLDWAEAHR | | |
| | | EQVRAVIRISHADRPGTEPQTLLDAARLAQ | | |
| | | AVTGHASRMPRLQAGDMLVEVMDPDAMPSE | | |
| | | RIGHRPDGLRADPGTDAGHGPSDGVTVHDT | | |
| | | WIGVRGATIPDCLDTLLDGTEPVQPDGQAT | | |
| | | PIRIIIPFDSPAEKENTMRELRILKDALRH | | |
| | | HR | | |
| 60 | 60 | MMRRARLFGPLLASGCEVLISGEGTRRVRL | | [″4'-phosphopantetheinyl |
| | | FDGERALVGNATAKRRREFTETRLLAHEAL | | transferase N-terminal |
| | | RRIGHDGPILKGSDGEPLWPSGIVGSLSHC | | domain″, ″4'- |
| | | PSLCVAAVASAERIRAVGVDVDDSDGLSDG | | phosphopantetheinyl |
| | | IMRFVESSEELASLRMASSVERRAAFCAKE | | transferase EntD |
| | | AASKALSALDGTGDFRKVSVSLKADGTFAA | | (siderophore |
| | | VRRDVTFRGQWRFYDRLVSAMVAVPSETV | | biosynthesis) (EntD) |
| | | | | (PDB:4QJK)″, 'Secondary |
| | | | | metabolites biosynthesis, |
| | | | | transport and catabolism', |
| | | | | 'enterobactin synthetase |
| | | | | component D |
| | | | | [EC:6.3.2.14 2.7.8.-]'] |
| 61 | 61 | MTIIESQSMPETVEKDRRLAKSGSLELCAI | 48.3 | ['ABC transporter', 'ABC- |
| | | SKTYHGKNGDFCAVQHTDLNIAPGTFVTLL | | type |
| | | GPSGCGKTTTLRMIAGFEQPTGGDILLDGE | | Fe3+\Vspermidine\Vputrescine |
| | | SILDMPADKRPMSMVFQSYALFPHLSVRGN | | transport systems, |
| | | VEFGLKLKKMDKALRCKKVDEALEMMGIEQ | | ATPase component |
| | | YADRYPHQLSGGQQQRVALARALVMEPKII | | (PotA) (PDB:1Z47)', |
| | | LFDEPLSNLDARLRVKMRGEIRALQRRLGI | | 'Amino acid transport and |
| | | TAIFVTHDQSEALTMADVIVVMSAGRVEQI | | metabolism', 'putative |
| | | GSPWDIYHHPVNRFVASFLGTSNFLEGQIQ | | spermidine\Vputrescine |
| | | SVENSGASDNAMAMYSVSTAFGDMAVAGVT | | transport system ATP- |
| | | GMQTGDKVNVVVRAEDLLVGGEAGAGRMAI | | binding protein'] |
| | | NCSVVSSAFDGQVVNYTLDTATGQMIGSAP | | |
| | | GSMAPAGSGAEVTAVFDCRALWCVPVEKHG | | |
| | | A | | |
| 62 | 62 | MFTDGESHKQLRRLVGTIINTRYHAINYTW | | ['Biotin biosynthesis', |
| | | PQINKNCDFTTEYARPYVCGILAQLVGVSV | | 'Cytochrome P450 (CypX) |
| | | EDISRMVSASETINSFLLRERLTLDDIEQV | | (PDB:3A4Z)', 'Secondary |
| | | AHSIEYAYQVVKEIEDKHVGEPLYIGNELL | | metabolites biosynthesis, |
| | | DLPQETRYPLIINLVTDGFAPFVAALDELA | | transport and |
| | | FNLLTHPYLEKELNARAEQISLESLRLFPP | | catabolism!!!Defense |
| | | FTTISRTCVHEIPFKEKIIRPGQLVILDLY | | mechanisms'] |
| | | SINRDPEVFPDPEKENLENTARAYSFGAAQ | | |
| | | HLCSGNPLVRKALEQVTRQSESLYKYKIQS | | |
| | | SCFKNSYGFTDMNLSIELK | | |
| 63 | 63 | MNDGISDPQLKGKIPVKLLHDSPYLDHKLD | 37.0 | ['ATP-dependent Clp |
| | | LYLQSNPNARPRDILVYLQSIESDARALLK | | protease, ATP-binding |
| | | SIRIPQNLTSSNSMETIIGREKEIQLLDIY | | subunit ClpA (ClpA) |
| | | LNRRYKNNVILIGEPGVGKTSLILYYFKSH | | (PDB:1KSF)', |
| | | HLPLMSVSAAEMLSGTKYRGEFEKRMQHVL | | 'Posttranslational |
| | | ETAQKEGSAIFFDEIHTLIHAGASEGGVSA | | modification, protein |
| | | ANLLKPIITRGDIKVVGATTPEEAKTLYAD | | turnover, chaperones'] |
| | | GAFERRFSFLKLKEPDMQTLRLIALNFVKE | | |
| | | SGDNQCFPSSLFEEIVAFLDEKFPNRHYPD | | |
| | | KLIDFIDFYLAANKQANFTLHEATVLFAES | | |
| | | QI | | |
| 64 | 64 | MFEEKDSDVILTILNIFKNPATVDDVSTAL | 27.9 | ['ThiF family', |
| | | LDTPYEVDDIASLVQRLFENNVIKEYKDIT | | 'Molybdopterin |
| | | ALDTTLTPKQLAKYDRQLRNYAVLPNFTIN | | biosynthesis', |
| | | DAIKQQERLDAASILILGAGGIGSYLAIGL | | 'Molybdopterin or thiamine |
| | | AQIGVGTLHLIDFDEIELSNTSRQVLYREK | | biosynthesis |
| | | DVGKSKIEVAVKNLTEVAPDATVVGHNLEV | | adenylyltransferase (ThiF) |
| | | TSVNSLTEELKSDQFDLIAVCADKPLGKLV | | (PDB:1ZUD) |
| | | YIVDEFSKLSGTPVLYGGPYADSKIFLGPL | | (PUBMED:32239579)', |
| | | IIPGKTKSYSELVPSSYADTSNPKVASINE | | 'Coenzyme transport and |
| | | YRETAIVDTDNALAAKMMEVEIIKYIGKLM | | metabolism'] |
| | | DPSVVERQIVLDTWNWSFHDGQFSK | | |
| 65 | 65 | MPGSTFQHHATRAENVWRVTPSRSATSPMD | | |
| | | SLPVTHARNARNSAMRLESSRLAVELDRQP | | |
| | | SHRHRCEPASVKPFLRIARPHNGHLLALED | | |
| | | LFLLMEQVNTKSQAPGRINDGNVNHTPTRK | | |
| | | TGVSSTLDLRHTSLYLTQNMSHI | | |
| 66 | 66 | MIKQDALTRRASIAYYVSTAFLIASKSFPH | | ['Major Facilitator |
| | | AVLTVLLLHKGLDLTEIMFVQTAFTIAVEL | | Superfamily', 'Predicted |
| | | FEFPSGVISDLYSRKIVYLASILAWVAACS | | arabinose efflux |
| | | VIVFGTGFAMMCVAWALYGIGEALASGTVD | | permease AraJ, MFS |
| | | ASLINLYKRLSPDPDEEIKTFKRISNQISM | | family (AraJ) |
| | | VSMIIGAMLGSALYFTIGYNIYAVAMVLAC | | (PDB:4LDS)', |
| | | AAALPIVLAFPKDEHDAREKKPTIMSQVRD | | 'Carbohydrate transport |
| | | GLSELRKDRRLTFLIGMAAVSQIFFQTHEN | | and metabolism'] |
| | | LWQAYLLLMGVKDKYLFVFYLVFQVIGIAA | | |
| | | YAIHIDGRLRRLLYLGIPVALIMPLLITSG | | |
| | | SRIVSIGAYCISVFIFMFLQYMCDVLFSIR | | |
| | | VSEERISTLITLNSTTCRIIGFLVLGLNGL | | |
| | | LLKQIKLTTLIVGSFEIATFLSILLGLLFM | | |
| | | LSFSKKKKE | | |
| 67 | 67 | MKRFFDRPDTHWRNLSGALHVYALPDANSY | | |
| | | LVKEARETASWLNGAVELAVQPVDYLHMTI | | |
| | | QRLDLYREEIPAEIWDELTNHLAMSVADIE | | |
| | | SFDVEYAPATVRAGAIEAVSDENPSWKRLV | | |
| | | DAVRESFCNVGLKHALVDPPFGPHYTVAYC | | |
| | | VQDTDAQRDDELCQLLVDAPATSMRVSSVD | | |
| | | LVAVDQSPEEGVFRFSSLMHWPLRGA | | |
| 68 | 68 | MEVRAGRPEYLGGRLLRYLPPFVACLPIQP | | |
| | | TGAASSQRMYSLRLMW | | |
| 69 | 69 | MQRKHRVSMADIAKETGVSAATVSRALNNH | 28.7 | ['Periplasmic binding |
| | | PKVSAEVRAAVLQTADRLGYIRNLGAASLA | | protein-like domain', |
| | | ASRSMTVGLLLRDMSSQFYGGVAAQVQMET | | 'DNA-binding |
| | | DAAGYDLLITIGGDDAESQMNAIRNLLGHD | | transcriptional regulator, |
| | | VGGIIVASGRIAEEVMEYSARFVPTVALSS | | LacI\VPurR family (PurR) |
| | | GLDMPSVGSVRIDPKCEADLARRVVVAGHR | | (PDB:1BDH)', |
| | | NVAVTASSNPLASTLHARTATFLTELIVAG | | 'Transcription', 'LacI family |
| | | AQTLIMSMPVEQGRYLREQVDRALEAKVTA | | transcriptional regulator'] |
| | | IMAGSDAIAVSIMEYLQELGLSCPDDISVT | | |
| | | GFDGVGALRSPLLGLTTVEQPMERLASAAV | | |
| | | GMITQYLTSGGSEEPISGTVINQLVMGRFV | | |
| | | PGRTLGSPKAV | | |
| 70 | 70 | MHSYKYSLLGVVAAVAMSLTAIAPASAEPI | | |
| | | ESSNLSSNSITLSSEDKMEISDILTSYGVD | | |
| | | EEKAQYLVSRYEHGYAWDSFTPGKQPIAAT | | |
| | | QRKTLYSVETVKTYEDGSIAVSTVPNFEAL | | |
| | | ADAPQTRGITGCQYHQSGSTRYWKNCDGTV | | |
| | | NLAVISMGFNFNYQNVNHSNPKITRYGPYH | | |
| | | HHIIGGALSNFREDRISDSQVRLSADLDVA | | |
| | | FRGFPAGWTAWMQVNVTGDNAWTSNN | | |
| 71 | 71 | MWDYARMAQIAGKLGGPARTGALIFGAGMI | | |
| | | LGGVIVDGAHQAMRQVDEESRRKADMAAQA | | |
| | | RRLENQVSGRDDN | | |
| 72 | 72 | MHANGLRSFWAEAIVVESGKRRDYCSRNTC | | |
| | | GAFMKFVAQCSPQK | | |
| 73 | 73 | MSSTRTIETVKASDIDEAFERGDDVRRYED | 41.3 | |
| | | MTKPRVIRPAKTKTRKVNLTLPDWMVESLD | | |
| | | AEADELAVSRNAVVNTWLAEKIAERRKEQR | | |
| | | LLTV | | |
| 74 | 74 | MEYAIQLSLGVSNMYAIFTVRCNTCLMTSE | | ['FCD domain', 'DNA- |
| | | TLGNMAASSETSLHDRLLDEWGMAVVSGTV | | binding transcriptional |
| | | SAGERLPEPDMDGNATPSRTVTREVTRVLE | | regulator, FadR family |
| | | SMGLVTVKRKAGATANPIEAWNILDPQVIQ | | (FadR) (PDB:1E2X)', |
| | | WRLRGPHRIDALHELSQLRAAVEPMSARLS | | 'Transcription'] |
| | | AANATPEHWATLTRAAIEMVAHSDHANESE | | |
| | | YLDADILFHRTLLEASGNLMFAALGDVIAS | | |
| | | TLTGRTQHELMPQVADQTALGWHTEVAALI | | |
| | | RKGDGDGAETAMRQIVDESDQAISHIAGTE | | |
| | | A | | |
| 75 | 75 | MNTLVLAEAAQAVQLNSTQLGISVVASIVV | | ['GntP family permease', |
| | | LILLVTVAKLHPFVSLLISALVVGIGSGYG | | 'H+\gluconate symporter |
| | | PVATVESFSTSFGSTMASVGILVGLGAMLG | | GntT or related |
| | | RVLMDTGAADSIVDTLLAKASPKMIPWTMA | | permease, GntP\VDsdX |
| | | LIGALIGLPMFFEVGLVVLVPVIILITRRS | | family (GntT)', |
| | | KLPLMRVAIPTLAGLSVMHACMPPQPGPLA | | Carbohydrate transport |
| | | ALSCFKNGSVGVTMMFGLPIAVITAALVGP | | and metabolism', |
| | | LFSKFAAKWVPVGAPENFDTGKGRVDADGN | | 'gluconate:H+ symporter, |
| | | PITTKPPFSLSVLCILVPAILMLGNAIFEI | | GntP family'] |
| | | VAPDQAGSDAVYAQILAFFGKPAIALGTAV | | |
| | | IFAMIVLGRTTHMSWKTVNDSLKAALPPIA | | |
| | | GILLIVGAGGGYKGVLVDTGIGDIIGKFVE | | |
| | | SSSIPIFLLAWLIAAFVRVATGSATVAIIT | | |
| | | TAGILGPVVEQMGVTTPAIALLVIAIGAGS | | |
| | | VFLSHVNDAGFWLIKEYFGLEVGETFKTWT | | |
| | | VLECLLSVVVLALVMICSIFVPLV | | |
| 76 | 76 | MQMGMIGLGRMGGNMVKRLRDGGHDIIGED | 34.8 | ['NAD binding domain of |
| | | MNPDSGRDVASLEDLVAALAAPRVVWVMVP | | 6-phosphogluconate |
| | | AGEPTDSTIARLGELLEPGDIVVDGGNSKY | | dehydrogenase', '6- |
| | | TEDREHAAALAEHGIGFLDCGVSGGVWGAA | | phosphogluconate |
| | | RGYALMIGGSDKDYAAVLPIFETLKPEGEY | | dehydrogenase |
| | | GLVHSGPVGGGHFAKMVHNGIEYGMMQAFG | | (decarboxylating) (YqeC) |
| | | EGFATMMRSEYVTNPAETMDSWREGSVVAS | | (PDB:4E21)', |
| | | WLLDLEDNATKDDPELKNVPAVANESGEAK | | 'Carbohydrate transport |
| | | WMIEAALELGVPVPTTAAALWQRQTSRGGG | | and metabolism', '6- |
| | | DDILRVVTALRAQFGGHVTKVDEIATH | | phosphogluconate |
| | | | | dehydrogenase |
| | | | | [EC:1.1.1.44 1.1.1.343]'] |
| 77 | 77 | MKLQRKALKTLKQWKTTPDHKPLLIRGARQ | 50.0 | ['AAA domain', 'Predicted |
| | | TGKTWLVNEFANGQYDNIVSVDFMQRPSLS | | ATPase, AAA+ |
| | | GIFEQDLDPQRIIRQLELAANQRILPGRTL | | superfamily', 'General |
| | | LFFDEIQESPLALTSLKYFTEQAPDYDIIA | | function prediction only', |
| | | TGSYMGISKHGKTSFPVGKVTMMNLHPLSF | | 'uncharacterized protein'] |
| | | VEYLDSIGQDMIADTIREGRFEDIPQALEP | | |
| | | QMNDLLKTYMWVGGMPAALSAHLDNGIPQD | | |
| | | VRAVQQDILNAYDLDFSKHAAYTLGERIRL | | |
| | | VWNTLPSQLAKENRKFVYGVVRQGARAREY | | |
| | | EEALTWLTDYGIITKVPCLDALHIPLTGYE | | |
| | | SLNTFKIYLEDTGILGALSGLDVNTLVNKS | | |
| | | KLFSEFKGAFVEQYVCQQLVAQGIKPRYWA | | |
| | | NPNPQGNAEIDFVMEQGDEVFPIEVKSSSN | | |
| | | IRARSLSYVCNRYGLHGIRIGEIGYRKQSW | | |
| | | LTNIPLWCVDGLGEYLKRQIEKSRAEA | | |
| 78 | 78 | MKLRKLFAGVAAAATLFGGMAFGATTANAA | 38.0 | |
| | | ATDAATITVNNAQVGYTYTGYKFATFDNVQ | | |
| | | GEAPNATSVEVNTVAAWKDAVYQAADAANG | | |
| | | NAAVPAEYAENPAAYVATFDAATARKFTDE | | |
| | | LTKHIPTGEQGTAAVNGVITATEGWFLVTS | | |
| | | KAETAGKSALVATQITSGGETYTKITLNTE | | |
| | | DGQHNIDALGRFNAKDENVPTPPTKTADGQ | | |
| | | GTVNVGDTVNYTITAVVPPAAAGYDTYKYT | | |
| | | ITDAASKGLNVAKGDADFVVVVKGGNADGT | | |
| | | DKTLAESTDYTLTQAGSASAVNGTVTTIAF | | |
| | | PNVKDYAGKTIQVTYKGVVTSDAVDQVTNT | | |
| | | ATVKNNNDQTGEGTPVVKKLGKFDFTKIGV | | |
| | | GSDAEGLAGAEFKVSADGGETFIKFSQDAN | | |
| | | GVYYPDANGNETLTSADGQGAQKTLGKVAV | | |
| | | RGLAEGTYTVQETKAPTGYAENFKVTFTVT | | |
| | | IGEDGGEGTLSADVLQQVNTTNKTVLNVKS | | |
| | | ITQLPLTGAAGTALFTVVAVLLAGVAATVE | | |
| | | AKSRSTKRALNA | | |
| 79 | 79 | MRDFELVKLSEDEFDKFSACHPQGNFQQTS | 47.9 | ['FemAB family', 'Lipid |
| | | AMGTLRKGEGKTVDYLGVKEHGELKAAGLL | | II:glycine glycyltransferase |
| | | QIIHAGGSTFALIHDGPMCDFDDKELLAFF | | (Peptidoglycan |
| | | VGKLKEYAKQGGAAQLDITPEAVYQLHTQK | | interpeptide bridge |
| | | GELEGSADDEMVANLLALGFDHVGGFSTGY | | formation enzyme) |
| | | TSVPRWRWVKDLTGIKDEAALTASYAKYRR | | (FmhB) (PDB:1LRZ)', 'Cell |
| | | RNVRIARESGVHTRRLERDELSLFHQLCEL | | wall\Vmembrane\Venvelope |
| | | SCEKQGFENRPLSFFEEMYDAFGDNIEYRV | | biogenesis', 'alanine |
| | | AEIHFDEYLKTWQDKLDKLNADKARIQKDL | | adding enzyme |
| | | ERSRTDKRTNQLNLQLASVDKNMPPVVKRV | | [EC:2.3.2.-]'] |
| | | QEAHDLLDQYGAVVPLDGSMFLYHPREVVC | | |
| | | TTSGADERFDKFYAPALMHHEMMVKCIERG | | |
| | | IPRYNLYGINGLFTPENNPGFGVLEFKQRF | | |
| | | NGFVEEMPGEFVLPVKPLVYAAKQLAHKLL | | |
| | | HR | | |
| 80 | 80 | MSIPSQHVKARLQNADFFEKCTFAFFLITF | | |
| | | VLKALTDGFFITQGVKGAITDSKYLTMGAA | | |
| | | IFFGIVYMVQRRRNRVFWNEFRQLITVALC | | |
| | | FVMATLVLVIAQNHFVQWQIRDILNLITPM | | |
| | | IFAYVMLNVLSFEQLFHGMKIALVESITGY | | |
| | | ITQLVLRGVTFGDIFASSFSDSTSPLESND | | |
| | | FSAIAIMFCFFFCYYRSSRWLTVLSTLYAI | | |
| | | ATFKRMAIIFAVIAFFFPMLENRDAELPKW | | |
| | | FSAISKIVFFGIAMFYCYLMLPTSTALQSA | | |
| | | LHLDIGEMTMGRSDFLASLINQGYQSFGFG | | |
| | | SVEGTIGHSLEMAFVRMTFELSPIAVLLFI | | |
| | | NNYWNITGRNLYCSLLMVFNFLNLTTADSI | | |
| | | SAMFAWAVCYILIGMVVYAHGPAVTAAKQS | | |
| | | RLFAKWQRG | | |
| 81 | 81 | MTMHSDNPRKAYITICTDDKYLPGVVALNR | | ['Glycosyl transferase |
| | | SLRTVESEYPLIVLTTGNMSESGVQTLANE | | family 8', 'Cell |
| | | SIRHLTAKNIVPSEYIRNLNIKNGSPNWSN | | wall\Vmembrane\Venvelope |
| | | TFFKLRIFGLSQFDTLVYLDSDMIVLRNID | | biogenesis'] |
| | | HLFDKLHLSAVAAGHHFNKTWNQLNSGLMV | | |
| | | FTPSIALEKNLVDLIEGEPSADMLNGQGIG | | |
| | | DQDIINHYFDDWDRQDNLHLPETYNQFISL | | |
| | | VPEYLRKGYLATTKDIYVVHFVGKVKPWNY | | |
| | | TIKEYLHMLLRALRWRSLAEFSIVRTENRL | | |
| | | LRK | | |
| 82 | 82 | MVSLQKVLPRVLPCIPGNDLLYRILRINAV | 42.9 | ['Glycosyl transferase |
| | | NAEPLACDVDALDELSDIHPRSSSPFVPDY | | family 2', |
| | | RVGGASDYDLSLIVPCYNVEDYIDECLTSI | | 'Glycosyltransferase |
| | | FGQETHYSMEVIAVDDGSTDSTADKLNQWK | | involved in cell wall |
| | | QRHDNLVVYRQKNAGLAAARNTGLDHARGS | | bisynthesis (WcaA) |
| | | NIMFLDSDDMLAPNAVELLMDTLTSSSADY | | (PDB:5MLZ)!!!CDP- |
| | | VSGSYVRVNESGKPISKPYQIGSCGMPWGR | | glycerol |
| | | VYRASVWDSLRFLEGYWFEDTLQAYCIVPF | | glycerophosphotransferase, |
| | | HRETRQPLAQTRYRIRGNSISHDSARRNKS | | TagB\VSpsB family |
| | | ADAYWVVEATLEQCRMLGLPIGQTLYEQTI | | (TagB)', 'Cell |
| | | GQFGALGASRIDGWSEANRRIFFLACSNLI | | wall\Vmembrane\Venvelope |
| | | TTTTEFTGLTTKRALVWRDMELALRTRNYR | | biogenesis!!!Cell |
| | | LWKLACYFAYIGR | | wall\Vmembrane\Venvelope |
| | | | | biogenesis!!!Lipid |
| | | | | transport and |
| | | | | metabolism'] |
| 83 | 83 | MTAPQTDIRRHYNDLDGFRAIAAFAVVVMH | | ['Acyltransferase family'] |
| | | VFLRGAYGADLAHGDGTDLLSLIQTIVSSL | | |
| | | GTFVTLFFIISGFGLCCGYYDRIKNAEITP | | |
| | | ERFYTKRIAKLLPFFALLVLLDIIGTGGKD | | |
| | | SLWEAFADITMVENLLPDLNIEVIGVGWAL | | |
| | | GVIFLFYFLFPFFVYTISTKRRAWLTFAVS | | |
| | | IALSLSCVFYFHKTNGNLDDRRFIYQAMFF | | |
| | | VAGGLLFLYKDRIGSMGKIGRIVTLVIAIG | | |
| | | ALPLLYVSGPAWTTNLRQLVLWVPWMVFAI | | |
| | | ASDHRIFSNRIAKFFSGISFEIYLSHLFIF | | |
| | | QVENMLHLTHLTGIPSVDYLMTLALVIVGV | | |
| | | TGFSVLAKRAIDYGWSLWRKRR | | |
| 84 | 84 | MKIVFVNPIVYTPENASIPKVDNITSTMSY | | ['Glycosyl transferases |
| | | DLCLAFQRAGIDMTLVAAEEWKPIRQTDFP | | group 1', |
| | | FHVVWMKSHWKRFFPIHRIPVNLGLIRYLK | | 'Glycosyltransferase |
| | | HSDADLVITSEVFSVDSLICSVFARHKTII | | involved in cell wall |
| | | WHEMAKHNRMGGGMLSRFWYNVIPKLFMRK | | bisynthesis (RfaB) |
| | | VLVAGRSEEARAFISRYCARVSETVIGHGV | | (PDB:2IV7)', 'Cell |
| | | NLDVFRTAFHKTNTFCVVSQLIDRKRIDGI | | wall\Vmembrane\Venvelope |
| | | IKAFDAYVRRYDADCKLYVIGDGDRRTDLE | | biogenesis'] |
| | | RLTQSLGLENSIEFMGQLEHDELQRYLSEA | | |
| | | KAMLVNTSKDNSMLSIVESIASATPVITTS | | |
| | | VPLNAAEIRSHELGIVKDGWNEDDLAYLDA | | |
| | | HLDELVEHCKVYRETLSTDYKVRQFMKLYD | | |
| | | GCIRPQRKG | | |
| 85 | 85 | MMHTISDSYGEWNGDQMTESIRPLVSVVVP | 39.5 | ['Glycosyl transferase |
| | | VYNTKPDDLRSCFASLSQAKDARLEIIAVD | | family 2', |
| | | DGSRAETAHLLDDIAAECSNTVHVIHKING | | 'Glycosyltransferase |
| | | GQSSARNRGIAEARGEYIEFVDSDDYVDWD | | involved in cell wall |
| | | AQQRVLETLTSHKPDILQINVVGMTEAGVY | | bisynthesis (WcaA) |
| | | FWPPKHGDGEYREIDKREIMTECAAMWAQL | | (PDB:5MLZ)', 'Cell |
| | | VKRELFETSGIVLCEGIHIGEDFASILSLA | | wall\Vmembrane\Venvelope |
| | | TVARSAAVLDVDLYYFIDHDSSITHIPHPQ | | biogenesis'] |
| | | MLLDITHAMDFVLEHVGDDLEKYHNEIEYQ | | |
| | | AIKQVRYAGVVRALDWEGIHSKVIPQLIEY | | |
| | | METHFPSWQRNPYYCQEAANQLKYRLLIGG | | |
| | | HYRLYILLHQGLHALRNSGGIRSVLLN | | |
| 86 | 86 | MADKSVFVDCFLSHNLGDDLFFFTLVSRYP | 36.2 | ['Polysaccharide pyruvyl |
| | | KVNFTVYADRSYEYLSNRFPNVKLITSVES | | transferase', |
| | | SSSRFGTADKIMRVCSAMRQRVALIREADA | | 'Polysaccharide pyruvyl |
| | | MVTIGGSIYMESKARGPKERLQRLYRSCKD | | transferase family protein |
| | | ASYAKAAGHYFILGANFGPYYSQQYLDSYR | | WcaK (colanic acid |
| | | RFFERRCDDVCFRETYSAGLFPSVKSVRSA | | biosynthesis) (WcaK)', |
| | | PDVLFTADLPSVPKRRQAFFSVVDLDNDGK | | 'Cell |
| | | FGALRDRRRQYEDWLLRSINECSQAGYDVV | | wall\Vmembrane\Venvelope |
| | | LASFSEPEGDVKAVSRLAEEASRQGSDVQP | | biogenesis', 'colanic |
| | | LFYTDNMDEVLRELAASEIVVGTRFHATIL | | acid\Vamylovoran |
| | | GLVAGARVLPIMYSDKTKHVLEDIHFDMTD | | biosynthesis protein'] |
| | | AVDLKRATDDELVAMSPVRDATSFDVHDVI | | |
| | | AAAQGQFAALDTYLSTITTS | | |
| 87 | 87 | MFANALAFTVQFGINFFLTPYIVSTLGSEA | 29.9 | ['Membrane protein |
| | | YGFIPLVNNIIGYASIITVALDSISARFIT | | involved in the export of |
| | | IEITRGNYQKANSYFNSVLLADTILALLLM | | O-antigen and teichoic |
| | | MPSLLFILKINDIINVPVDLLVDVQLTFFF | | acid (RfbX)', 'Cell |
| | | AFLTFFVNLIFTVIGCCYYVKNRVDLNAKR | | wall\Vmembrane\Venvelope |
| | | SIESNIIRACILIALFTLNKPHIFFVTITT | | biogenesis'] |
| | | AVVAMYLFACNVHYSRKLTPELRVDLHKES | | |
| | | FLVIKEMLSTGVWNSINALSSTLLTGLDLL | | |
| | | LANIFLGASQSGEYALVKTVPNFICQLVIV | | |
| | | VLSAFVPEFNILYAKGDKKELLKSVDESLR | | |
| | | IMGYLVTIPIGFLIVFGKEFFSAWVPGQNV | | |
| | | DLLQQLSIMTLLPLIAICGTDSITKIYTVT | | |
| | | NKLRTPAIFMIIMGILNATGDYLLFTFTSL | | |
| | | GIWVIPCVSFIVNIIIQLLFTPIFGAYCLH | | |
| | | LKWNTFYLSIARSCSCAIVVISVSLLFKFI | | |
| | | IQPQGWFSLFLTGFLCSILSLIFSFFIAFD | | |
| | | KDVRVRIVAILKSKLQKQ | | |
| 88 | 88 | MLGFSSSRYLAAPVMRLRMLSEARFGKIQL | | |
| | | HTRREAFKYRINIIIQIDYRFHIHIINNLT | | |
| | | TLNILNCGAKLQRPTHNLENISNLYCFCSL | | |
| | | LFNIATILTLTSLSKAIKKLKIKDNIEHKN | | |
| | | PVRKRENHPCG | | |
| 89 | 89 | MTHDATDQAQYSTPVSRPIYIRHLDVTDRS | 57.5 | |
| | | DYLLYAALALLPVDGTVLGWYMPFWTPISP | | |
| | | WLLMLYTALNWRLIPQVYRRFRTFFLFPLL | | |
| | | LVALSSFGWFTVAFHPLPALWSLLGIGGAL | | |
| | | ACLASLGIAVTIKHLDWRQMIRIILIAYWF | | |
| | | AFAVGVVQFLSIKLDITFVRDWFSDLMSRE | | |
| | | YITADSAWGGNRPQFLFAEPSYIGMHLYGV | | |
| | | LLPLMWLMRRRDRIYARRLRDLIIVFAAGS | | |
| | | IIMGAGVRIILDTGVALVIAIIVDTDFKNH | | |
| | | KQARLAWGTFGVMAVAGVAVALLNSRIRAI | | |
| | | LAQGPLLGDDSTSARISQTMTPLVALIKHP | | |
| | | ANLLLGFGSGNIVEANRQGTTAAYAILNGP | | |
| | | DAKVPWWVWKSLTPTNVFTMSSYTSFITEF | | |
| | | GLIGFIVLVSIILRHITRQHAWSKTTTCWL | | |
| | | ILTAYLYLQFEGYAFYVIPLLIWTSPKIEG | | |
| | | RC | | |
| 90 | 90 | MNKYKNLLLNTGLFAFSQFATKLITFFLVP | 48.7 | ['Membrane protein |
| | | LYTYYMTTEQFGVTDMSSTVIALLLPLVTL | | involved in the export of |
| | | SASDAVLRFVIDDKKNQDKYISLGVGLIAC | | O-antigen and teichoic |
| | | SVIVVAVSLPLLDLQFLGGLGKYKGLFFLC | | acid (RfbX)', 'Cell |
| | | YVVSACQYFCGLLARALNQLKLIPAASIIS | | wall\Vmembrane\Venvelope |
| | | TLVTGVLAVLLIAKMGYATEGYFWSLIIGN | | biogenesis'] |
| | | ACGALTFVFAGKQYHHIQFIRSSMDLILLK | | |
| | | KMLAYSIPMIPNALFWWIGVSINRFFITGM | | |
| | | IGIGASGLFAAAQKIPNLLNTFSGIFQQAW | | |
| | | QLSAFQEFKKKDISGFFATVFKLYHGGIAI | | |
| | | VSTGIIALAQWLASFMLQKDFYYAWPMISV | | |
| | | MILAFYFNILNAYYGTIYTSAMKTKHLMTT | | |
| | | TVAGAVSSVICTWLLIPIAGIYGAGIAMVI | | |
| | | SNALVLVLRVITAKKILVFKVDWPSVIVTM | | |
| | | LLLISQCVVSLIHWPSYLVVSWVLTIAICG | | |
| | | LQVFSCRSVMSRAIAMVRHR | | |
| 91 | 91 | MLVEDGIVTIEVYLKSFLAHELSVYGGEFG | | ['Abi-like protein', 'Abortive |
| | | YMRQEGLPKLSYDAHLECLASLRTTEMKSS | | infection bacteriophage |
| | | LPYLRHFRNTYSNPLPPYWMIVGCLSYGTL | | resistance protein (AbiF)', |
| | | KGDFYQGAPDSIKRKLATRLHIENPNPNPE | | 'Defense mechanisms'] |
| | | VRGDAKILSNWLETIRQARNMTAHHDREWN | | |
| | | ETSTRIAPKLPKHRSGAHAQDWWGNDWDAF | | |
| | | RKASGPAAFLTMENFLLTQIDGPSWREKFV | | |
| | | SLMNRYPQIPKSDMGFPDNWESLPLWQGLS | | |
| | | L | | |
| 92 | 92 | MLFLIRGNDKWPKCFAEELRLRQFIETLKL | | |
| | | LGKIILIMMESFRVEICNDSLRMLTLITSL | | |
| | | SFTKIESNGIYFFPLIS | | |
| 93 | 93 | MVKNVGSVVRWQVAKLALVQGMQAMSSAFF | | ['Major Facilitator |
| | | TCGVVFTGAASSDSLGLALVLMFKTLPTLV | | Superfamily', 'Predicted |
| | | MAFLGGVLADRLPRKTLASGMLGGLAISYG | | arabinose efflux |
| | | VGTWVVQLSGLGWPVQAISLAAGIIGAVGS | | permease AraJ, MFS |
| | | PALFALLPSIAPPEDIVRANGLIRTERNAG | | family (AraJ) |
| | | SVVGPLLGAWLAQLISPSFLFLDGAVCLAV | | (PDB:4LDS)', |
| | | SMPLVLSLKLMPSCDDEANNNDDDASMISA | | 'Carbohydrate transport |
| | | LRSIPSLFHTYIWLAVGVPFWALFLAVQSG | | and metabolism'] |
| | | ATDVTMPLWVVQESGRGAWSLMASITSSGY | | |
| | | ICGSLIALKLERPRHMFSKSVLFGALAIMP | | |
| | | IFVVGTLDIQVLWYVASFVAGLGLELSGVF | | |
| | | WGSTMQTCVDKRHMGRVSSIDYAISFGLIP | | |
| | | LAYGLYGFTGTIHAAVVLTVSSSIMIVLVI | | |
| | | IVFPFCYLIDHRSNSSGIINSSDV | | |
| 94 | 94 | MLHDSVAQCLTSIRLIAQRQSNDPQNDAWE | | ['Signal transduction |
| | | KIDKIARKGLEATRDIIDTMIQDNNENGIP | | histidine kinase ComP |
| | | IESSEWWLAVKRLTDECDYILHQHGFTGTT | | (ComP) (PDB:4GT8)', |
| | | EIINGGFTVQQQVTVKNIDILHEVLRELCS | | 'Signal transduction |
| | | NIIKHAPKYSEFQSSIALQKSQTEIVMSNS | | mechanisms'] |
| | | MSTIPDEERSGRGLESRRRKLNLIGGEIDH | | |
| | | EVDGDTWIVYARIPLTMVSPDKAATESMED | | |
| | | NEHEKYSPRKRGKTRSVECQREQMARKEHK | | |
| | | AQK | | |
| 95 | 95 | MRGNHRAINSNPMWMKPNNISYADVVGLSV | | |
| | | QSIIEKNKESNTIINLNGCFLMLLFIITCP | | |
| | | DSYCLAHCFRFELTTSSIKICCCTSLSNLH | | |
| | | AIRGF | | |
| 96 | 96 | MNNEPREEDSSVECYHSTAVASAATHIALL | 27.1 | ['DNA-binding response |
| | | DNDAIVLKGLQQIIEYNHLGSITWTTRSGR | | regulator, NarL\VFixJ |
| | | EAVQRCSSAVDTPHLLLFDMSLDGMSGIDV | | family, contains REC and |
| | | CRQIRKRSASVLLLGITAFPLERYISRLIQ | | HTH domains (CitB) |
| | | AGAQGLVAKDEERQIAEVTRWVLNKGGCGN | | (PDB:1A04)', 'Signal |
| | | GFETARNAHMRLKHETNDIRMLLSDREEEI | | transduction |
| | | MILLSKGLSISEAANRMQIGQASAATYLNR | | mechanisms!!!Transcription'] |
| | | ARRKLKAETVRQAVAIWTGDYEQ | | |
| 97 | 97 | MIRIGLTGGIAAGKSTVSTRLRELGAALID | 52.9 | ['Dephospho-CoA kinase', |
| | | YDELARRVVEPGGVGLRRIAECFGPDALTD | | 'Pantothenate\VCoA |
| | | QGRLNRRWIAEHVFAGPDSERMRRKLDDIE | | biosynthesis', |
| | | HPLIYDLALSRERQAVADNPDAMVVHDVPL | | 'Dephospho-CoA kinase |
| | | LAEVLDAMPMRFDHIVTVEAPEQVRVDRMV | | (CoaE) (PDB:1JJV)', |
| | | STRGMTRDDALARIRHQSSPEQRRVIADAV | | 'Coenzyme transport and |
| | | IDSTQPMGRMLEAVDALYEQWLAES | | metabolism', 'dephospho- |
| | | | | CoA kinase [EC:2.7.1.24]'] |
| 98 | 98 | MSALLITHNLTYRIDDRTLWEGLNLTFSPG | 47.1 | ['ABC transporter', 'ABC- |
| | | DMVALTGESGCGKTTLLNVLGLLEEPSSGT | | type lipoprotein export |
| | | ITYDGQTIASRKGRRLMHRNVMGFMFQNYA | | system, ATPase |
| | | LVEQWTVNRNLILALRSVGIPSADRSRLIR | | component (LoID) |
| | | RALRAVNLTGYGNRPIYTLSGGEQQRVAIA | | (PDB:5GKO)', 'Cell |
| | | RLLIRQSLRVILADEPTAALDADNRAMVMR | | wall\Vmembrane\Venvelope |
| | | HLRDFADNGAIVIYTTHNEETAALADRIIA | | biogenesis'] |
| | | L | | |
| 99 | 99 | MHWTYKIASILAVALITLAAGFYAQNNEET | | ['Uncharacterized |
| | | YPSGPSYDIGISDAHGQPLSTLTGLAATHH | | conserved protein, |
| | | VVLARVSYEPDGHGTNRRVISIFGALDGNG | | DUF1430 domain', |
| | | MHANSPYPDYGFEPRTRVQRGDRFTDPLGR | | 'Function unknown'] |
| | | WLLYGSPRDTASVAKSIRRQGFELNRATPI | | |
| | | GITQITKQFFSNSIAQVIFAGLAVVFVSGA | | |
| | | LSVSTASRVCAIQALYGMRTTGIIMRQFLR | | |
| | | HAFFFIICVCIGWMTWISIGAIFWPFASPL | | |
| | | GFAGQVFLSIIIATTCMALVALSLSIALVR | | |
| | | VLVPNTLKLIQGKRPLRFLMASGCIMAIIV | | |
| | | LALSSASLNVTNFKWRQSQTLKTTLEHQLS | | |
| | | PNDGFQLQLWYSSDQNRARSMPSWNDFVEQ | | |
| | | TSQSEHTRFASFRLGCTWVDSSQDPQPCIL | | |
| | | MDSRTARLHHLIRNNTTLARISVIMPENEQ | | |
| | | WNSESITNNVLRAYSFEQSLAAEEGKSLPA | | |
| | | INRMSISIESRPRDAVLSAFDTPSNTDGLS | | |
| | | SVPVVVIDPSLLSGDTTTSMVSTGGMTEDY | | |
| | | SSRHQLLEILREKGVDSLVASVVNRHDEIQ | | |
| | | TRLARTTQEMNYFSITACISITCLLGGAIM | | |
| | | VALTLCTLRRQIMFVEYMHGAPSYLRFQSI | | |
| | | LFLAAALCSASLPIQLLIGGYNAVSTTSVS | | |
| | | LLFIVVSLATTVLYDSRLRADSIKHP | | |
| 100 | 100 | MKNGRPIRLALVDNDRCSAEMMALLIGRTI | 35.2 | ['DNA-binding response |
| | | PEAHMLWVTDNPSLALERCLFDPRKPDILI | | regulator, NarL\VFixJ |
| | | CDLMMDGLNGVRLTERIRQRNVQVGVIVVT | | family, contains REC and |
| | | SYDLATYGEDIACCGAQALISKRDFAATIR | | HTH domains (CitB) |
| | | EAVKSVSDGGTYPHGWGLHSLEETLHTVDA | | (PDB:1A04)', 'Signal |
| | | AQPEADGARLESDRELAVLRLYAHHVPTVE | | transduction |
| | | IARRLGIGVETVYSYVKRAMRKVGVTRRGE | | mechanisms!!!Transcription'] |
| | | LLDYCERYHVL | | |
| 101 | 101 | MSCDGSRFLRRRFRDFGKSWHGEKIAALAC | 28.1 | ['Signal transduction |
| | | ALCIAADTVIESCINPVWDAWALWVGLLFV | | histidine kinase ComP |
| | | ILSLLCVAFPFGGNIALAISWCVVFPLPVD | | (ComP) (PDB:4GT8)', |
| | | LSMSVSVVIAEPLIVLSYQRIWCGVVLAVA | | Signal transduction |
| | | VTVSRVAQLIWQYGLPMGWDAGALVSVVPW | | mechanisms'] |
| | | TVMPALACVGIGLLLNWHHREGERESEARG | | |
| | | RAESLDLAARLHDATTNDLSYLIMSIDRIM | | |
| | | SEHPSQGESMDLPLLREVAQRALDQTHDVI | | |
| | | AVLAKHNVGTARIPRCHARRSGEAIVPIGA | | |
| | | GRFEAEIERHRRELATLGFRGEVVVSDPFD | | |
| | | LLSRFDEKTIQLTRSLLEETFANIAKHADR | | |
| | | EQGYVFAIQVRQDGLYVSVADVPAKMADGA | | |
| | | LAESPRTLGMGFGMSHLRQSITRSGGWLRV | | |
| | | QEEDGYWSCLAYIPIYHRDTA | | |

### HMO Utilization Genes

HMO utilization genes were detected in PB-STR-220 using a functional genomics pipeline built with CENTRIFUGE™ (Kim et al. (2016) Genome Research 26:1721-1729). The pipeline annotates a genome with functional annotations including KEGG ORTHOLOGY database (KO) numbers. Lists of KO numbers associated with HMO utilization genes and HMO utilization gene clusters were obtained from published research (Henrick et al. (2021) Cell 184: P3884-3898). The Blon gene accession IDs associated with each observed KO value in PB-STR-220 are listed in Table 11 where the genes are grouped by the HMO utilization gene clusters from Henrick et al.:

TABLE 11

| | Number of | |
|---|---|---|
| Cluster | Genes | BLON IDs |
| H1 | 5 | Blon_2331, Blon_2332, Blon_2334, Blon_2357, Blon_2360 |
| H3 | 1 | Blon_0423 |
| H4 | 9 | Blon_0625, Blon_0641, Blon_0643, Blon_0644, Blon_0647, |
| | | Blon_0648, Blon_0649, Blon_0650, Blon_0651 |
| H5 | 7 | Blon_2171, Blon_2172, Blon_2173, Blon_2174, Blon_2175, |
| | | Blon_2176, Blon_2177 |

### Bacteriocins

Using ANTISMASH™ (Blin et al. (2023) Nucleic Acids Research 51: W46-W50) bacterial version, the PB-STR-220 genome was searched for bacteriocins, peptides known to have antimicrobial and immunological properties relevant to the infant gut environment (Benítez-chao D. et al. (2021) Frontiers in Microbiology 12), and the observed signatures are listed in Table 29.

TABLE 29
observed bacteriocin signatures for Persephone strains.

| Strain | Bacteriocin Signatures |
|---|---|
| PB-STR-220 | lanthipeptide-class-ii |
| PB-STR-093 | Lactococcin_972_1, lanthipeptide-class- |
| | ii, lanthipeptide-class-iii, |
| | Lactococcin_972_2, YcaO, lanthipeptide- |
| | class-v |
| PB-STR-083 | Lactococcin_972_1, lanthipeptide-class- |
| | iii, thiopeptide |

### Antimicrobial Resistance Genes

The ORFs found in the genome for strain PB-STR-220 were BLAST searched against the NCBI Antimicrobial Resistance Database and no antimicrobial resistance genes were observed. Antimicrobial resistance genes are increasingly common in infants (Shan Y. et al. (2019) Nature 574:117-121) and pose potential health issues (Samarra A. et al. (2023) Gut Microbes 15:2194797).

### Virulence Factors

The ORFs found in the genome for strain PB-STR-220 were BLAST searched against the VFDB (Virulence Factor Database) and no virulence genes were observed.

PB-STR-207: B. longum

Persephone strain PB-STR-207 is a member of the species B. longum. Comparative genomic analysis of PB-STR-207 was done with the published B. longum genomes found in Table 28. The type-strain of B. longum is GCF_000196555.1. PB-STR-207 is differentiated from the type-strain by the following values:
accession: GCF_000196555.1,
ani: 98.7%,
coverage: 84.4%,
product: 83.3%,

The most similar published genome to PB-STR-207 is GCF_000772485.1 (determined by the strain with the highest ANIb product). PB-STR-207 is differentiated from GCF_000772485.1 by the following values:
accession: GCF_000772485.1,
ani: 98.8,
coverage: 89.0%,
product: 88.0%,

Table 12 provides a list of the unique open reading frames (ORFs) from PB-STR-207. These ORFs were determined to be unique by BLAST searches with the ORFs from the above list of published B. longum genomes. If an ORF from PB-STR-207 has no corresponding ORF in any of the published genomes (with a sequence identity greater than 60%) it is considered unique and included in the table. If an ORF had a sequence identity greater than 20% but less than 60%, the highest sequence identity to an external strain is shown in pident (percentage of identical matches). Where functional annotations were possible, they are included in the table.

TABLE 12

| | SEQ | | | |
|---|---|---|---|---|
| | ID | | | |
| index | NO | AA Sequence | pident | function annotations |
| 1 | 102 | MLRDGSSSRSVPESGGRSVMAVVATLSGAG | 46.9 | |
| | | VSFAPLYERIWGGMMLHPNCPAIGRMPYYL | | |
| | | SGQATDDYGVCVTSPLWFMTVTALSVVAIL | | |
| | | CLTVAGVQGFSRHRLCSRCPVLVRSHVLFT | | |
| | | YSYNTGRHTDFCPALSATDNAGLVAYCIGR | | |
| | | EALGLLTVVLCVLPLESLLSAIMAITLAAH | | |
| | | DATPPA | | |
| 2 | 103 | MFTAQRETIWRVVFPSNNVGMDEEFRIAAA | 40.1 | ['Galactofuranosyltransferase 2 |
| | | PIYGSGYDASGGSLSNAELHKEITGRTSLE | | N-terminal', |
| | | VPPEHTYSTASYYNAFPAAYWAQWTNVQQV | | 'Glycosyltransferase, GT2 |
| | | VLNLTVAGEGSVTVHRSDADANDYIVAKKS | | family (WcaE) (PDB:2Z86)', |
| | | VNATATSPQVVQIPVPIYGMAKGGWLWFDI | | 'Carbohydrate transport and |
| | | EASADASVTLSDASWQTEVSAKRNLTASLA | | metabolism', |
| | | ITTMNKPEWCIRQFNLLADMADMNLIDAVY | | 'galactofuranosylgalacto- |
| | | VVDQGSNLVEEHEGFAAAKVKLGDKLRIIQ | | furanosylrhamnosyl-N- |
| | | QGNVGGSGGFARGMYEVEHHGESGYALLLD | | acetylglucosaminyl-diphospho- |
| | | DDTVLEPESVSRAIAFANHCEKPTLVGGNM | | decaprenol beta-1,5V1,6- |
| | | LFLSEPTRICALAEVFDPQTISWGTAVKES | | galactofuranosyltransferase |
| | | RYDDLASTSFLDKQYLHRRVDADYNAWWMC | | [EC:2.4.1.288]'] |
| | | LIPTEVIRKIGLSYPFFIKNDDVEYGVRAQ | | |
| | | RAGYRTVTVPGVCLWHQSFVDKDDQLDWQA | | |
| | | YYHIRNRTIMGLLYANQQYKRNILKEMVRF | | |
| | | TLSATAKMRYSAVALHQAAMRDVIAGPEHV | | |
| | | GTILETKLPEIREIRSGFADSNMVPVEDLP | | |
| | | DTLRAQDEKFAHLHDLSRAEAILGIGLHQI | | |
| | | MPPRANRSAVIDGYMEPTKVHCLIDNGHST | | |
| | | HTVPADQLDSLVLVADDASDHWRALGLMDS | | |
| | | AVFVDPDRRKGILLTRQPVRAITGFIRACG | | |
| | | LYVKVIANWRTYQRQYRKAFATMVSPEWWQ | | |
| | | RYFTK | | |
| 3 | 104 | MPSIRFANVLMEITPRALSYPTMYYHTNQP | 24.3 | ['Glycosyltransferase, GT2 |
| | | VRVNPDTHEWFVEGAGTIDFTTYFNSLSTM | | family (WcaE) (PDB:2Z86)', |
| | | KLLKYTRATGFHLHLEVKGNACTITQTKAY | | 'Carbohydrate transport and |
| | | RLSSSPEIDPTVFAKVQASNKWQSIDLDLT | | metabolism', |
| | | VDENMVLAGFQIETTGAIVVRDAYYTLDID | | 'galactofuranosylgalacto- |
| | | GELTDIELSLSTTTFKKESYITKNIELVKK | | furanosylrhamnosyl-N- |
| | | EILGSDNDIAKHFRMHVIDNGCTLPYKELS | | acetylglucosaminyl-diphospho- |
| | | TDKVTISPNENVGGAGGFARGMIESMEQDV | | decaprenol beta-1,5\V1,6- |
| | | PATHVLLMDDDVEVSPESIMRTYNLLRIVK | | galactofuranosyltransferase |
| | | PEYSEAFVSGAMLNYEDVQDMKEDTGFIDP | | [EC:2.4.1.288]'] |
| | | QIGICVAAKIPLQVTKFVDIVENEVYDENL | | |
| | | RVGDGRRYAAWWYCCIPMSVIKRNGMPLPV | | |
| | | FVRYDDVEYGIRCNPTFMTMNGLCIWHSKF | | |
| | | EIRYNAAVERYQSIRNGMIAQMTTGLAPSI | | |
| | | DTFLRELHDQVDLELKKFNYTDAELALKGF | | |
| | | EDFLKGPDFIKQPIVQEKFVQANQEKEKLV | | |
| | | SFPELQQMADDMGLEGFDVSKLTRQEIDND | | |
| | | KPRSIQQRAFDELTINGQRLLHSSIHGAKG | | |
| | | TPGKRYALISSAGWIYPAGSIHGENIIIAI | | |
| | | DWFNRRGTIRTKNLKQYNAVTKRYKRDLAY | | |
| | | FKKNRERLSAEYKAASKELTSIQYWKQYLG | | |
| | | MK | | |
| 4 | 105 | MRRVITYGTFDLLHYGHINLLKRAKAYGDY | | ['Cytidylyltransferase-like', |
| | | LIVGLSTDEFNAGKGKKAYFSYDQRKELLE | | 'Riboflavin\VFAD biosynthesis', |
| | | SLRYVDLVIPEQTWEQKRNDILLYQVDTFV | | 'Glycerol-3-phosphate |
| | | MGSDWAGKFDDLSDICDVIYLPRTPEISSS | | cytidylyltransferase, |
| | | KIKNDLEHRN | | cytidylyltransferase family |
| | | | | (TagD) (PDB:2B7L)', 'Cell |
| | | | | wall\membrane\Venvelope |
| | | | | biogenesis', 'glycerol-3- |
| | | | | phosphate cytidylyltransferase |
| | | | | [EC:2.7.7.39]'] |
| 5 | 106 | MSFLNTIAKHLPASKRAVAQTLNEVKMLRE | | ['LicD family', |
| | | HVDVLYGQLYARIEQADYGINDNLNYKVDT | | 'Phosphorylcholine metabolism |
| | | ILTPHLNDLGTALDAHDAHMKIFAWENYRH | | protein LicD (LicD)', 'Lipid |
| | | KGESLSAAKQRFFMSLPPATGSTRLLQEGC | | transport and metabolism', |
| | | AQLMTEFDQLCRDNNLPYWLDFGSLLGAVR | | 'lipopolysaccharide |
| | | HHGFIPWDDDTDLGMMREDIDRLQGIVQHD | | cholinephosphotransferase |
| | | SRYRLSLVYDAIAFCRQIREMSSDTSNPCF | | [EC:2.7.8.-]'] |
| | | VDIFIYDYTDSTAIEVYDRRQHIRTELLDA | | |
| | | LRQSQFRAWHDLVYLSETSDGAAEIQQVES | | |
| | | RYQREMEESGIVVSKENASGIMYGIDNVDN | | |
| | | SSVRLYKLDDMFPTTCLTFEDHEYQAPHTP | | |
| | | MTVLTRNYGDIYSLPRDINSHFIHVDPALL | | |
| | | QQDNVQESIQDSLSEIPISKNEE | | |
| 6 | 107 | MLRELKLRLNNKNISVWVFAFIIMLLVLIG | 39.8 | ['Family of unknown function |
| | | RAVDDGSQGGSKRSLAYAAVFGLLSWLIAY | | (DUF6020)'] |
| | | LIFHWFDCLTGSRVENGDSASLPLRQRVMS | | |
| | | VDYWAATQDAISFTTSIKKYGLVCLIGVIF | | |
| | | CWLPWVITCWPGVMRDDTIAQFMQSSGYHF | | |
| | | YYTQHPLFDTLVFGFFWELGFALHHVLLGL | | |
| | | GIYVLVQTFSFAIGVMLVLCYLRKIGAARS | | |
| | | LLLAIFLFFAFCPAIVGAVPTMAKDSLHTV | | |
| | | FLLPLSIIYVEIFLTRGKVLHRRPVCVMLV | | |
| | | LLVALCMLSKRTATVAILCAFCVLVASVKK | | |
| | | NRLKVVASMIIAMVLAQGIIEPALVRVTHA | | |
| | | EVSPGKEVMGLIMMPVARIQSISPERISPQ | | |
| | | ERSALSSLLNIDKAGKTYTNYRIDETSWTI | | |
| | | NNEASIAQKIKGIGAWVSLGVHNPGEYVKA | | |
| | | FGNLMLGWFYPQVGVFYGSNSDGLESDQYM | | |
| | | IQWDSFVRPPLSAENVLHDMRGTGQKSSLL | | |
| | | MRAADAGQQIAINPILNAYAYYATYIPLLL | | |
| | | LIYGMSRKRWIAVGAGSLLGFNVLVLYLSP | | |
| | | LVFAWYLLPVTFILPLFFGITGCIAEKQ | | |
| 7 | 108 | MAKKMVMPVAWAQDVDCWLETLKAAGFSDD | 51.7 | |
| | | TVRSRRYKIARLCRELPSPMETTGDQITRV | | |
| | | FAAHDWKPETRKGYRNTIAGFYRWFYETGR | | |
| | | RGDNPTAKVPKVKKPQAHPHPCPDKYMAGS | | |
| | | SDRCNT | | |
| 8 | 109 | MKKIIAAAVTVTTVLSLAACGGNTAVDKSD | | |
| | | CLDVPQDVLNVVASGSDSSGLKPETGKAVK | | |
| | | GDTEGTYWLAMKFTADGENGDTETGIWLVS | | |
| | | GLDAASAAPVMSVDGFAKQFTHWPTQINGT | | |
| | | ELNGTEEKAKAAAACLA | | |
| 9 | 110 | MAIQGTFEGFSEIGNRQGFMETRTRANLKT | | |
| | | FFDGKTVTEAADTYAALMTAIAHNIDSYLT | | |
| | | LGKNISTLADSYNNAFDHLRELYPEAPELD | | |
| | | ENLAALLTEAKA | | |
| 10 | 111 | MRPRKGPIVRGIRTVLAAPFAVLAFALATV | | |
| | | AMFSARAAMWISAGYRGAVKVEAEL | | |
| 11 | 112 | MPVFTKKSHNHFDVDHFEVDNSFGFRITVD | 54.1 | |
| | | GDYFAFAGMSLGDLVTINQHIAEAIRKGRR | | |
| | | NAL | | |
| 12 | 113 | MYDVSVTKQTRPEITTGELIKRLLAFNGLT | | |
| | | QQDMADAIGCSRSSVSQKCAGHVILTADEI | | |
| | | AKTADLLNVSADVLLGRKPLEVK | | |
| 13 | 114 | MYLYEGKALCNQFFSGTQPTEKYAIMNLDI | | |

### HMO Utilization Genes

HMO utilization genes were detected in PB-STR-207 using a functional genomics pipeline built with CENTRIFUGE™ (Kim et al. (2016) Genome Research 26:1721-1729). The pipeline annotates a genome with functional annotations including KEGG ORTHOLOGY™ database (KO) numbers. Lists of KO numbers associated with HMO utilization genes and HMO utilization gene clusters were obtained from published research (Henrick et al. (2021) Cell 184: P3884-3898). The Blon gene accession IDs associated with each observed KO value in PB-STR-207 are listed in Table 13 where the genes are grouped by the HMO utilization gene clusters from Henrick et al.:

TABLE 13

| | Number of | |
|---|---|---|
| Cluster | Genes | BLON IDs |
| H1 | 15 | Blon_2331, Blon_2332, Blon_2334, Blon_2342, Blon_2343, |
| | | Blon_2344, Blon_2345, Blon_2346, Blon_2347, Blon_2350, |
| | | Blon_2351, Blon_2352, Blon_2354, Blon_2357, Blon_2360 |
| H3 | 1 | Blon_0423 |
| H4 | 5 | Blon_0625, Blon_0641, Blon_0644, Blon_0647, Blon_0648 |
| H5 | 7 | Blon_2171, Blon_2172, Blon_2173, Blon_2174, Blon_2175, |
| | | Blon_2176, Blon_2177 |

### Bacteriocins

Using ANTISMASH™ (Blin et al. (2023) Nucleic Acids Research 51: W46-W50) bacterial version, the PB-STR-207 genome was searched for bacteriocins, peptides known to have antimicrobial and immunological properties relevant to the infant gut environment (Benítez-chao D. et al. (2021) Frontiers in Microbiology 12), but no bacteriocin signatures were observed.

### Antimicrobial Resistance Genes

The ORFs found in the genome for strain PB-STR-207 were BLAST searched against the NCBI Antimicrobial Resistance Database and no antimicrobial resistance genes were observed. Antimicrobial resistance genes are increasingly common in infants (Shan Y. et al. (2019) Nature 574:117-121) and pose potential health issues (Samarra A. et al. (2023) Gut Microbes 15:2194797).

### Virulence Factors

The ORFs found in the genome for strain PB-STR-207 were BLAST searched against the VFDB (Virulence Factor Database) and no virulence genes were observed.

PB-STR-215: B. longum

Persephone strain PB-STR-215 is a member of the species B. longum. Comparative genomic analysis of PB-STR-215 was done with the published B. longum genomes listed in Table 28. The type-strain of B. longum is GCF_000196555.1. PB-STR-215 is differentiated from the type-strain by the following:
accession: GCF_000196555.1,
ani: 98.5%,
coverage: 78.4%,
product: 77.2%,

The most similar published genome to PB-STR-215 is GCF_000219455.1 (determined by the strain with the highest ANIb product). PB-STR-215 is differentiated from GCF_000219455.1 by the following values:
accession: GCF_000219455.1,
ani: 98.8,
coverage: 86.4%,
product: 85.3%,

Table 14 provides a list of the unique open reading frames (ORFs) from PB-STR-215. These ORFs were determined to be unique by BLAST searches with the ORFs from the above list of published B. longum genomes. If an ORF from PB-STR-215 has no corresponding ORF in any of the published genomes (with a sequence identity greater than 60%) it is considered unique and included in the table. If an ORF had a sequence identity greater than 20% but less than 60%, the highest sequence identity to an external strain is shown in pident. Where functional annotations were possible, they are included in the table.

TABLE 14

| | SEQ | | | |
|---|---|---|---|---|
| | ID | | | |
| index | NO | AA Sequence | pident | function annotations |
| 1 | 115 | MNDYADSITAERSRKVIGGYGDHEKIDSGF | | ['Adenine specific DNA |
| | | SFYELGPVLFDADGELNAAVPAEEIRKYIW | | methylase Mod (Mod) |
| | | YSETKAPYVDMTAEHPYLLGVLGETVYYLA | | (PDB:4ZCF)', 'Replication, |
| | | YKPDGETTLGPRLLRLVPRRGAPTVVYADR | | recombination and repair'] |
| | | CVFDDDKLNELNVVFKQIPRQIARI | | |
| 2 | 116 | MEGSGMKPNTYTLNIDQWKFIVFTDLDRMD | | |
| | | RTSFVSIAPGIAVRADYRIRAMEDRIGKYD | | |
| | | IRLHMGYSEEEQRIVLRNCEIGTTRELKIR | | |
| | | DIARLPIEQIIRSYRPPLWSYEITDTGTNI | | |
| | | FGPLPDWEHDVLSSVDFPTLRKQGPTPDTL | | |
| | | KWASRVYSVTQLNKGPATKRLTEVFGIPLR | | |
| | | TASHWLTLMKERVPESVSMRLPSPITIHDE | | |
| | | TKPDTASGTALKKLLE | | |
| 3 | 117 | MTSEHCNVTDGFSDRDRELLAMFDMTEEQV | 57.4 | |
| | | REAEMIAESETIPDGLVGPVYYGRHHTDAS | | |
| | | RLGS | | |
| 4 | 118 | MFRSLGYTTEVTPASRDGGYDILLRGRDGV | | |
| | | MSIVECKCYAHGATA | | |
| 5 | 119 | MPVGQHAVHLLVRVRRYECMACARSWTDDL | | ['Transposase', 'Mobilome: |
| | | THMADEGRRLTDAAVWWAVAEVVLKSKSVL | | prophages, transposons'] |
| | | ACARDLHCSWGVLNRAVLEKGADVLAADLR | | |
| | | RLDGVEAIGVDGHVWRHTRTGGRYVTVIVD | | |
| | | LTPRRHGRPA | | |
| 6 | 120 | MSKSNCEPALLSSLETLQHNLQDAGMLRMK | | |
| | | ASLYSEAAVRDVLRLLEAK | | |
| 7 | 121 | MKLQRKALKTLKQWKTTPDHKPLLIRGARQ | 49.8 | ['AAA domain', 'Predicted |
| | | TGKTWLVNEFANGQYDSIVSVDFMQRPSLS | | ATPase, AAA+ superfamily', |
| | | GIFEQDLDPQRIIRQLELAANQRILPGRTL | | 'General function prediction |
| | | LFFDEIQESPLALTSLKYFTEQAPDYDIIA | | only', 'uncharacterized protein'] |
| | | TGSYMGISKHGKTSFPVGKVTMMNLHPLSF | | |
| | | VEYLDSIGQDMIADTIREGRFEDIPQALEP | | |
| | | QMNDLLKTYMWVGGMPAALSAHLDNGIPQD | | |
| | | VRAVQQDILNAYDLDESKHAAYTLGERIRL | | |
| | | VWNTLPSQLAKENRKFVYGVVRQGARAREY | | |
| | | EEALTWLTDYGIITKVPCLDALHIPLTGYE | | |
| | | SLNTFKIYLEDTGILGALSGLDVNTLVNKS | | |
| | | KLFSEFKGAFVEQYVCQQLVAQGIKPRYWA | | |
| | | NPNPQGNAKIDFVMEQGDEVFPIEVKSSSN | | |
| | | IRARSLSYVCNRYGLHGIRIGEIGYRKQSW | | |
| | | LTNIPLWCVDGLGEYLKRQIEKSRAEA | | |
| 8 | 122 | MNETATFDAFFESIKLDSLNEYQNVLDCIG | 36.2 | ['Adenylyl\VGuanylyl and |
| | | KKLNDSFYHIDSKNEHLIIVGSIGRGTAVP | | SMODS C-terminal sensor |
| | | GTSDLDVLFDLPEDVFHTFDSYKSNGQSAL | | domain'] |
| | | LQKVKEAVKERYPKTDVRGDGQAVVISFES | | |
| | | KNFTVDLVPAFRQTDGSFKYPDSHNGGSWK | | |
| | | TTNPIPEQEACTTLFAQTDNAALHICNALR | | |
| | | IWKNNVGFHFKGLLIDTLVGKYFDQKNSIP | | |
| | | LNSYDLFIDVFENLSLVNRNQSYWHAIGSN | | |
| | | QQVTNDDKGAFVPKSQKALNILRAASSESD | | |
| | | REEALIKLFGKTIAKCMVDSIHQENERKEL | | |
| | | KKYSITNNEEFIEDLFTIDISNYLEIDCKV | | |
| | | TQDGWRTKSLRDMLSKHLPLLPRKQLDFHI | | |
| | | VRCDVKSPYEIYWKVRNCGEEAFKRNCIRG | | |
| | | QITEGTLDAPLREHADFQGPHFVECYAVKN | | |
| | | GICVARSRIDVPISETGEII | | |
| 9 | 123 | MLFNLCSKKEKHMTEKTQIINKRLDQILYG | | ['SMODS and SLOG- |
| | | TEWTHKIHEKVADTFEITDRLLTFISVLTT | | associating 2TM effector |
| | | AFSGSGILAAVESNNQSLRIAAAILAAISL | | domain family 4'] |
| | | FSTLLTKSFRESVRATEQRHAAREFLSIRE | | |
| | | CIKNLQVKINTDNISVDETLTEVFQLSDAY | | |
| | | TSACIKAPSTNFFAKYLAEKEFLRSSSELT | | |
| | | SSYKKERNTDE | | |
| 10 | 124 | MSFTLPSAYSLFSSRNEAKSCSPPVVGDQP | | |
| | | FGRAARVPDAGQGQGHPAQLPAGLQTGPPA | | |
| | | GQFEDVGLHVEQAPLDPRIRPRGLRGLEDA | | |
| | | APAVAYEHVGRRDACHQALPCRRFLAFGDM | | |
| | | PADHVPAGHRDQDHRVAVQVDAVHMHHMMH | | |
| | | LVHQRHGRPQAPHELAPAAQRACRQPVLGL | | |
| | | RLLREQPVQTAPQITGAVVARLGA | | |
| 11 | 125 | MVRTEDACEIIKYALQNEIKVYLDGGWGVD | | ['lincosamide |
| | | ALLKRESRIHNDIDLFVELKHYHDYIYVIK | | nucleotidyltransferase |
| | | QHGFEEVNTDYTTDGHTVWKDDKQRIIDLH | | A\VC\VD\VE'] |
| | | CFEFTDDGIVYEGDIFPSKTFSGIGKVGDI | | |
| | | TVSCIEPLSQVMLHLGYEHDKNDVHDVMLL | | |
| | | CETFQIAIPDEYKEK | | |
| 12 | 126 | MLQIKKLNLTHKKDLRIILNDENLVLNDGD | 22.7 | ['ABC transporter', 'ATPase |
| | | KAVIIGEEGNGKSTLMKWIYNPSLVENYIE | | components of ABC |
| | | ADGERIMGHERLGYLPQEMLDEDKEKTIYE | | transporters with duplicated |
| | | YFSEEEIFWEKTPKELSVIAGKFGMKNDFF | | ATPase domains (Uup) |
| | | YSNQTMGSLSGGEKVKTQLMRLFIRDVSVL | | (PDB:5ZXD)', 'General function |
| | | LLDEPSNDIDIATLTLLEKIINDWKHIVLF | | prediction only'] |
| | | ISHDETLIERTANMVIHIEQIIRKTKARYT | | |
| | | VAKLPYRRYVEERLHKFEIQKQRALSDRRE | | |
| | | KKIRDEKYQRVMQSVQGALRSCTRQAPSVA | | |
| | | KNLKDKMHTVKAMERRFEKEDENMTQMPEQ | | |
| | | EEAIFVKLGDENSHIPAGKTVIEYELSKLV | | |
| | | TPDGKRILAEGIHLKIKGSEKICMIGANGA | | |
| | | GKTTLLKKIAEELLNRNDIKAEYMPQTYED | | |
| | | LLDLDVTPVDYLDKTGDKEERTRIRTYLGS | | |
| | | LKYTPDEMEHPIRELSGGQKAKVLLLRMSL | | |
| | | SGANVLILDEPTRNESPLSGPVIRKMLREF | | |
| | | PGAVISISHDRKYIEEVCDKIYQLNPNGLQ | | |
| | | LIGD | | |
| 13 | 127 | MTKTIFEEMGGTYRQVGDYLLPNITVPAEE | | ['Transposon-encoded protein |
| | | EIEPIGLWGKRHARHLKEHYKVLYMNLLTS | | TnpV'] |
| | | GKLHSYLAEVDKQAEDMFLRLVKEYADRQD | | |
| | | VTEQLKKDNPYEWIGRMNNIQACVREVVGT | | |
| | | ELIYT | | |
| 14 | 128 | MQRTISAMVGKGSVNHNSRKFRAENVDGTR | | ['Plasmid recombination |
| | | THLNIDYCNENIKTVYHELFDEALERYNAK | | enzyme', 'Uncharacterized |
| | | QIRSDRKIKDYYEKIRSSKQEKPFHEIILQ | | protein, contains DUF3084 |
| | | VGGKGNMNADTENGELAKQILDEYYQGFQE | | domain', 'Function unknown'] |
| | | RNPQLRVFSAHLHMDEATPHLHIDFVPFTT | | |
| | | GSKRGLDTRVSLKQALATQGFKGGSRGDTE | | |
| | | WSQWIQSEKEQLAAVMERYGIEWEHLGTHE | | |
| | | KHLSVLDYKKQEREKEVAALGAKIEQKQIE | | |
| | | FDVLSERVLNYDKAKDELSNLEIELDTAPK | | |
| | | YQLPEPEKFMTAKAYKTKMAEPVVRKLKQL | | |
| | | VKTVLARCFEGWDNYHRLNTANAQLYRTNQ | | |
| | | RLEKVNERLTEENKILKAENKDYSLLRKVE | | |
| | | GRKQIDDLLEQARTVKGRKRDNTRSR | | |
| 15 | 129 | MENKKEMTIPNVSAATDAEQSLSKCTDNSI | | ['AAA domain', 'RecA-family |
| | | VNQDTDFKGYEQSFEEMQREILRQLDPSYL | | ATPase (RepA)', 'Replication, |
| | | KTVSMTTLYDTVFEVQTPLIDGLLQRGTYL | | recombination and repair'] |
| | | FVGSPKVGKSFMMAQLAYHISTGTPLWEYK | | |
| | | VRKATVLYFALEDDYPRLQKRLFQMFGAKE | | |
| | | TGNLYFATECKTVNGGLEEQIRGEMREHPD | | |
| | | TGLIIIDTLKRVREAGGADYSYASDYDVVA | | |
| | | RLKALADSYKVSMLIVHHTRKQKSEDIFDM | | |
| | | ISGTNGLMGAADGAFVLSKDKRTSNNATLD | | |
| | | VAGRDQQDMKIHLVRDSERLVWNFAKSETE | | |
| | | MWKEPPEPLLEKIADTLFSESDRWEGTASE | | |
| | | LCERLAVDIKPNVLSLRLSINASRLFRDYG | | |
| | | IRYQNSRTHDGRKVSLWKETEQTA | | |
| 16 | 130 | MSAKNRDNKNRWRNITVGFRVSPEENELIN | | |
| | | RAVALSGLPKQEYCYRRCLNQDVVVQGNPR | | |
| | | VYKALKTEFATVLAELKRIEAGKGVDDELL | | |
| | | SVIELISIILGGLKGEDANGE | | |
| 17 | 131 | MARYVNWKGERKQKCKRGFATKREAQEWER | 25.0 | ['Phage integrase, N-terminal |
| | | MFKLQTSSDLDMSFEAFTELYINDVKNRLK | | SAM-like domain', 'Site-specific |
| | | ENTWLTKEHIIRTKILPYFGKLKISEISTK | | recombinase XerD (XerD) |
| | | EIITWQNEMLAYRDEKKKPYSQTYLKTLHN | | (PDB:1A0P)', 'Replication, |
| | | QLSAIFNHAVRYYELRSNPAAKVGNMGREE | | recombination and repair', |
| | | HKEMLFWTKEEYKKESFEMMDKPVSFYAFE | | 'integrase'] |
| | | MLYWCGIREGELLALTPADENEDKETVTIN | | |
| | | KSYQRLKGQDVITSPKTKKSNRTIKMPKFL | | |
| | | CEEMKEYLGMLYGLKKKDRIFTVTKSYLHH | | |
| | | EMDRGAKAAGVKRIRIHDLRHSHISLLIDM | | |
| | | GFSAVAIADRVGHESIDITYQYAHLFPSKQ | | |
| | | IEMAEKLDDLGKGDFENVS | | |
| 18 | 132 | MENRFIRAEDVAQELNVSKPYAYKLIRQLN | | |
| | | EELKAKGFITIAGRVNRQYFYERLYGAGKG | | |
| | | EM | | |
| 19 | 133 | MAISERIHFFRLMRGMTQKYLGTAIGFPEK | | ['Transcriptional regulator, |
| | | SADVRLAQYETGTRKPKADLTNALAQVLDV | | contains XRE-family HTH |
| | | SPQALDVPDIDSYIGLMHTLFTLEDIYGLT | | domain (HipB) (PDB:1ADR)', |
| | | VSEADGEVCLKVNKDKGREAYELLKMLYAW | | 'Transcription'] |
| | | KEQADKLSSEEINREEYDNWRYHYPEFDTT | | |
| | | QRWAKVPSQELSDALVEAFKDHLKDK | | |

### HMO Utilization Genes

HMO utilization genes were detected in PB-STR-215 using a functional genomics pipeline built with CENTRIFUGE™ (Kim et al. (2016) Genome Research 26:1721-1729). The pipeline annotates a genome with functional annotations including KEGG ORTHOLOGY database (KO) numbers. Lists of KO numbers associated with HMO utilization genes and HMO utilization gene clusters were obtained from published research (Henrick et al. (2021) Cell 184: P3884-3898). The Blon gene accession IDs associated with each observed KO value in PB-STR-215 are listed in Table 15 where the genes are grouped by the HMO utilization gene clusters from Henrick et al.

TABLE 15

| | Number of | |
|---|---|---|
| Cluster | Genes | BLON IDs |
| H1 | 5 | Blon_2331, Blon_2332, Blon_2334, Blon_2357, Blon_2360 |
| H3 | 1 | Blon_0423 |
| H4 | 5 | Blon_0625, Blon_0641, Blon_0644, Blon_0647, Blon_0648 |
| H5 | 7 | Blon_2171, Blon_2172, Blon_2173, Blon_2174, Blon_2175, |
| | | Blon_2176, Blon_2177 |

### Bacteriocins

Using ANTISMASH™ (Blin et al. (2023) Nucleic Acids Research 51: W46-W50) bacterial version, the PB-STR-215 genome was searched for bacteriocins, peptides known to have antimicrobial and immunological properties relevant to the infant gut environment (Benítez-chao D. et al. (2021) Frontiers in Microbiology 12), but no bacteriocin signatures were observed.

### Antimicrobial Resistance Genes

The ORFs found in the genome for strain PB-STR-215 were BLAST searched against the NCBI Antimicrobial Resistance Database. Antimicrobial resistance genes are increasingly common in infants (Shan Y. et al. (2019) Nature 574:117-121) and pose potential health issues (Samarra A. et al. (2023) Gut Microbes 15:2194797). There were 2 antimicrobial gene signatures observed. Table 16 describes each of these signatures:

TABLE 16

| class | sseqid | pident | product_name |
|---|---|---|---|
| LINCOSAMIDE | WP_063851341.1 | 60.4 | lincosamide nucleotidyltransferase |
| | | | Lnu(C) |
| TETRACYCLINE | WP_063856423.1 | 97.1 | tetracycline resistance ribosomal |
| | | | protection protein Tet(W) |

### Virulence Factors

The ORFs found in the genome for strain PB-STR-215 were BLAST searched against the VFDB (Virulence Factor Database) and no virulence genes were observed.

PB-STR-093: B. infantis

Persephone strain PB-STR-093 is a member of the species B. infantis. Comparative genomic analysis of PB-STR-093 was done with the published B. infantis genomes listed in Table 28. The type-strain of B. infantis is GCF_000269965.1. PB-STR-093 is differentiated from the type-strain by the following values:
accession: GCF_000269965.1,
ani: 97.8%,
coverage: 81.7%,
product: 79.9%,

The most similar published genome to PB-STR-093 is GCF_001281305.1 (determined by the strain with the highest ANIb product). PB-STR-093 is differentiated from GCF_001281305.1 by the following values:
accession: GCF_001281305.1,
ani: 100.0,
coverage: 99.5%,
product: 99.4%,

Table 17 provides a list of the unique open reading frames (ORFs) from PB-STR-093. These ORFs were determined to be unique by BLAST searches with the ORFs from the above list of published B. infantis genomes. If an ORF from PB-STR-093 has no corresponding ORF in any of the published genomes (with a sequence identity greater than 60%) it is considered unique and included in the table. If an ORF had a sequence identity greater than 20% but less than 60%, the highest sequence identity to an external strain is shown in pident. Where functional annotations were possible, they are included in the table.

TABLE 17

| | SEQ | | | |
|---|---|---|---|---|
| | ID | | | |
| index | NO | AA Sequence | pident | function annotations |
| 1 | 134 | MIDVPSDAPNFTFTVANESPSRLLTWPEAS | 59.6 | |
| | | LPATAVTGRIKASTRRARPWAAPVGRVVLV | | |
| | | ARARMDLDERAPAVGCHADFGVPPSPEDAD | | |
| 2 | 135 | MNRAGGSSGSLFPHWTLRLPLRDQKRMIDP | | |
| | | DPLFLLYWLLRGPSCGRDCGVVPGRRRATR | | |
| | | ICQYARNILQACSGKPAGNAVSWHLLMHLA | | |
| | | DMRALSVGAGPRFDSGTTPRLRYPTRWPSI | | |
| | | RRRRAKYWYCTDSQTAQRRQRTNLWRNTDS | | |
| | | GKAYRRQIARLWHTTDATRPTTIGNATNIG | | |
| | | PPPTLASLSVGAGPFHESVTAPMVSERRHR | | |
| | | DAVWISTDAA | | |
| 3 | 136 | MSATAPNYNHTVTACFVGYITQAVINNEMP | | ['Fucose permease (FucP) |
| | | LLFVTFAATLGIDMARLSALITVNFVTOLV | | (PDB:3O7P)', 'Major Facilitator |
| | | VDVLAGKFVDRIGYKPCIIAAHLAALAGLL | | Superfamily', 'Carbohydrate |
| | | ALGLLPTRVPDPYLAILAAIFLYALGGGLI | | transport and metabolism'] |
| | | EVMVSPIVEACPSEHKAKAMSLLHSFYCWG | | |
| | | QLGTVAISTLFLFAFGTGSWPVLACLWAIV | | |
| | | PAIGIAMFAGAPMPRIVPEGTATMRFADLS | | |
| | | KKPVFYLMFLMMLCAGAAEQGMSQWASAFA | | |
| | | ESGLGVTKVIGDLAGPAAFALMMGLSRTIY | | |
| | | GVLGHRLDLTAFIASSSVLCVAMYLTAALT | | |
| | | TAPVLGLLACALTGFSVGIMWPGTFSMAAD | | |
| | | AMPGGGTLMFALLAVAGDLGCAGGPAVVGL | | |
| | | VASANGDSLKTGLLFGSMFALVLLACVVAA | | |
| | | RKTVVGEREPLH | | |
| 4 | 137 | MTSASDRYQEKSDKKEHTMRNSSTPPLLRL | 36.5 | ['ABC-type sugar transport |
| | | EDICVKFGFVEALKSVNLSIQRQEVIAIVG | | system, ATPase component |
| | | DNGAGKSTLIKVIAGFLOPGFGHIYLNGEQ | | (MglA)', 'ABC transporter', |
| | | VTIPSIREADRMGIASVFQGQEFCDNLDVA | | 'Carbohydrate transport and |
| | | SNLFLGKEINQIGIRDDDSMNSRARSVLKT | | metabolism', 'D-xylose transport |
| | | LSSAIRVGSPIASLSVGORQTVAIARTLLN | | system ATP-binding protein |
| | | DPQLILLDEPTAALSVMQSAEVLAYIKRLR | | [EC:7.5.2.10]'] |
| | | SEGRSVVMVCHDLPDVFAVSDRIVVIRQGH | | |
| | | VTGVHRTVETSYEEIIAEIAGVTTEHEYEE | | |
| | | IAENPKFDSMVRQRKLIDRTISAAVSHGTG | | |
| | | HDSPLD | | |
| 5 | 138 | MATRLFGSQTSLREANRANLLASIHKFGAM | 27.5 | ['Sugar kinase of the |
| | | TQVELAEVTGLSTATVSTLVHOLVDEDQLE | | NBD\VHSP70 family, may |
| | | TKSTVRNGRRATLVTLARHQGLGVGLWIAR | | contain an N-terminal HTH |
| | | RHLTLSIVDFSKSIIAEHTLPLPLGHKADT | | domain (NagC) (PDB:1WOQ)', |
| | | TLERAMLLINETLSSIDAEASELVGIGVAV | | 'ROK family', 'Carbohydrate |
| | | AAPVATSDHTIAIPGILPGWDGVDITSPLR | | transport and |
| | | TAFNVPVYVDNDANFAAYGESRMGVAAGKR | | metabolism!!!Transcription', |
| | | NFVYISASDGVGAGIVINGEIMHGVTGLAG | | 'glucokinase [EC:2.7.1.2]'] |
| | | EIGHIQVDPLGAICSCGNRGCLDTVVAENR | | |
| | | LVQLLSVTHGNMTLDDLVSFANEGDPGCRR | | |
| | | IIADAAVRIGQVAADLCISVDPEVIVLGGK | | |
| | | LAMTGDVFIQPFNEALQRMLFPDAVAPIDV | | |
| | | LVSSHPDDNCALGGALCAIEFSVRNDVSQ | | |
| 6 | 139 | MKFAKKIVAVVAGVAMCAGLAACGGSRSGQ | 48.1 | ['ABC-type xylose transport |
| | | ATGGDAKIEKGATIGISMPTKSEERWNKDG | | system, periplasmic component |
| | | NNLKAKLEKAGYKVILSFADDKPAQQNADI | | (XylF) (PDB:4YWH)', |
| | | ENMVNNDAKIVVVASKDGTAVGPAVEKARD | | Periplasmic binding protein |
| | | AGAKVIAYDRLIMNTDAVDYYATFQLEQVG | | domain', 'Carbohydrate |
| | | VLEATYLIDQLKLKDGATGPFNIELFTGSP | | transport and metabolism', |
| | | \|DDNNAKYFFKGAWDLLQPYFEKGVLVSPSQ | | 'putative multiple sugar transport |
| | | HGQGGVTKDFTVEDWQKISVMSWKTEQAQK | | system substrate-binding |
| | | DMESILDSTYAHGEKLDAVLTPYDGIAQGV | | protein'] |
| | | INAIESKRPDMKPGTDSWPYITGQDAMEIA | | |
| | | VANIAKDKQGETVFKDVNKLADAVYDMVVE | | |
| | | IAEGKEVSGLNGKENNNNIDVPSKLLDPQN | | |
| | | ITKDNLQDLVTANYITQDREDELTK | | |
| 7 | 140 | MKLTARSTSRMYALTCLACVIWLWQSLVEA | 41.4 | |
| | | NNDGSLFNWATIVESLCLLVVIGWSGWNAV | | |
| | | AGWNAKETEAATAGAKDDEGSTDR | | |
| 8 | 141 | MCDSRTDTKTGLLPVNEVRSLLDVCWKAKA | | ['DNA-binding transcriptional |
| | | ITELMPALPKGLKPRYVHVIDAVWHINETN | | regulator, MarR family (MarR) |
| | | GQEIGTARVSDVSAFLGVTTPSVTKLVGEM | | (PDB:1JGS)', 'Transcription'] |
| | | VELGLVVKHMDAADRRAVTLTLTERGLDIR | | |
| | | RVYVEEYHAHLSQLLGGLTVEQCETTVRTL | | |
| | | TEALRLMQQDANNR | | |
| 9 | 142 | MHRIFRETLCSRQRHHPTHRNFPMTPHDQL | | |
| | | GTGPTAESLSTSLSTAITNIACIAPPRHRS | | |
| | | LINQHREHIHRQICDAFLLEHVGFREHHRL | | |
| | | VNGVAVSAQRIVKRRMRTVISARLNLQGON | | |
| | | ITIIGLYQEIQFANSFFRKVIQIGESMRGK | | |
| | | FLSHDILIDSPMFIAA | | |

### HMO Utilization Genes

HMO utilization genes were detected in PB-STR-093 using a functional genomics pipeline built with CENTRIFUGE™ (Kim et al. (2016) Genome Research 26:1721-1729). The pipeline annotates a genome with functional annotations including KEGG ORTHOLOGY database (KO) numbers. Lists of KO numbers associated with HMO utilization genes and HMO utilization gene clusters were obtained from published research (Henrick et al. (2021) Cell 184: P3884-3898). The Blon gene accession IDs associated with each observed KO value in PB-STR-093 are listed in Table 18 where the genes are grouped by the HMO utilization gene clusters from Henrick et al.:

TABLE 18

| | Number of | |
|---|---|---|
| Cluster | Genes | BLON IDs |
| H1 | 20 | Blon_2331, Blon_2332, Blon_2334, Blon_2336, Blon_2342, |
| | | Blon_2343, Blon_2344, Blon_2345, Blon_2346, Blon_2347, |
| | | Blon_2348, Blon_2350, Blon_2351, Blon_2352, Blon_2354, |
| | | Blon_2355, Blon_2357, Blon_2359, Blon_2360 |
| H2 | 4 | Blon_0243, Blon_0244, Blon_0245, Blon_0248 |
| H3 | 4 | Blon_0247, Blon_0423, Blon_0425, Blon_0426 |
| H4 | 12 | Blon_0625, Blon_0641, Blon_0642, Blon_0643, Blon_0644, |
| | | Blon_0645, Blon_0646, Blon_0647, Blon_0648, Blon_0649, |
| | | Blon_0650, Blon_0651 |
| H5 | 7 | Blon_2171, Blon_2172, Blon_2173, Blon_2174, Blon_2175, |
| | | Blon_2176, Blon_2177 |
| Urease | 12 | Blon_0104, Blon_0105, Blon_0106, Blon_0107, Blon_0108, |
| | | Blon_0109, Blon_0110, Blon_0111, Blon_0112 BLIJ_0113, |
| | | Blon_0113, Blon_0114, Blon_0115 |

### Bacteriocins

Using ANTISMASH™ (Blin et al. (2023) Nucleic Acids Research 51: W46-W50) bacterial version, the PB-STR-093 genome was searched for bacteriocins, peptides known to have antimicrobial and immunological properties relevant to the infant gut environment (Benítez-chao D. et al. (2021) Frontiers in Microbiology 12), with 6 signatures observed. The observed signatures are listed in Table 29.

### Antimicrobial Resistance Genes

The ORFs found in the genome for strain PB-STR-093 were BLAST searched against the NCBI Antimicrobial Resistance Database and no antimicrobial resistance genes were observed. Antimicrobial resistance genes are increasingly common in infants (Shan Y. et al. (2019) Nature 574:117-121) and pose potential health issues (Samarra A. et al. (2023) Gut Microbes 15:2194797).

### Virulence Factors

The ORFs found in the genome for strain PB-STR-093 were BLAST searched against the VFDB (Virulence Factor Database) and no virulence genes were observed.

PB-STR-083: B. infantis

Persephone strain PB-STR-083 is a member of the species B. infantis. Comparative genomic analysis of PB-STR-083 was done with the published B. infantis genomes listed in Table 28. The type-strain of B. infantis is GCF_000269965.1. PB-STR-083 is differentiated from the type-strain by the following values:
accession: GCF_000269965.1,
ani: 98.0%,
coverage: 81.1%,
product: 79.5%,

The most similar published genome to PB-STR-083 is GCA_920939435.1 (determined by the strain with the highest ANIb product). PB-STR-083 is differentiated from GCA_920939435.1 by the following values:
accession: GCA_920939435.1,
ani: 98.0,
coverage: 83.4%,
product: 81.8%,

Table 19 provides a list of the unique open reading frames (ORFs) from PB-STR-083. These ORFs were determined to be unique by BLAST searches with the ORFs from the above list of published B. infantis genomes. If an ORF from PB-STR-083 has no corresponding ORF in any of the published genomes (with a sequence identity greater than 60%) it is considered unique and included in the table. If an ORF had a sequence identity greater than 20% but less than 60%, the highest sequence identity to an external strain is shown in pident. Where functional annotations were possible, they are included in the table.

TABLE 19

| | SEQ | | | |
|---|---|---|---|---|
| | ID | | | |
| index | NO | AA Sequence | pident | function annotations |
| 1 | 143 | MNVVTQRISALVKDEGLTCAQLGSLLGLSK | | |
| | | TSANGKLLGRIGWTTSDIVVLSEHFHVSTD | | |
| | | YLLGFDADHEEVA | | |
| 2 | 144 | MAGGIYEQSEWPDSRWDCVTVCDGCHTVVW | | |
| | | STFWDDYDSAARDAYFARNGWRNYCVPGDT | | |
| | | EILELCPACAVRALRRSETRGLADSWLRPT | | |
| | | HAYTHAFREVDAQLSARERMVAGLLLTEGR | | |
| | | AS | | |
| 3 | 145 | MSNEVSCDSVARRVNGENPLDVLVGVSTPY | | |
| | | GPLLTDDYGWSIRFAEYGDGPCRFGDRPIQ | | |
| | | VMKPYEWLADRVYVEDDEAVAPFKDDVARF | | |
| | | VVEACRCFVGGDGDHDRSVVLCREGVAEQL | | |
| | | DLSSDAFTPMVGSDGNAVSFSYQPCDRLRC | | |
| | | VCSTNPQKGLGVFHVWTERGTTYQAVLGPC | | |
| | | AYERRPEKALTLPDELWSRNESWMRDFFEQ | | |
| | | ETSDFLCLGVVSRRTNIRFVEGGGAMV | | |
| 4 | 146 | MNDFTKAFRMSCSVFPECNRDFQAPVWTFP | | |
| | | VVAAFARHNGPGSVDSRRLASMMAHPSMEG | | |
| | | RLA | | |
| 5 | 147 | MSNIALSWAFKCHVGNASAKAVLVYLADRA | 27.1 | ['Mobilome: prophages, |
| | | DDDGTAAYPKIATIVNVTELSERTVRTALK | | transposons'] |
| | | TLQERGFIRRGDQRYARLGKGGRNRLPQYC | | |
| | | QIVWDLAVESDPSTLEWIKETHTAEHDPKT | | |
| | | MGNTVDPAASTIMENGESKDVTPENAGTKP | | |
| | | IPSTANLAGLENDPEPALQISHLQHCESCT | | |
| | | PSTANAAGLLYKDKTLQVNPPSKPSFPSAP | | |
| | | \|TGHLPASGATAAEKNKTEQLDEDDTEIAEA | | |
| | | AGRVLASLGAHRSMLGLATPSPTKADRKAI | | |
| | | IGLYRRLVDQGAQWPTLVMVGAIGFAMNGD | | |
| | | WWPKRIRTGRALARHWDELNDDMILAAGRT | | |
| | | DGDAHAQTVPAAVPEPDAVPWLPDWAVETL | | |
| | | AELDGQDATAGGEATA | | |
| 6 | 148 | MTTRIDCSEAGFSEFLLANPQLDGHADLIW | | |
| | | QLHAVYWRNKRLGHPKAVGLLIQYARAWAA | | |
| | | RNPGETAIGRLQARKTPMTQGRRP | | |
| 7 | 149 | MNGRDMMPACARIAAVDPAMADRMWNTTTD | | |
| | | DDGRDLVDERLRGKGRVLCAACPMRLDCIS | | |
| | | RALVNGWKDKAVYGGLDYASRWILARLIAR | | |
| | | DLHIADGGLHRIPQSRVRDWLAEHPDWAER | | |
| | | MRRDGRDYWRRTKRRQRSRREYTHDDPLSL | | |
| | | PTEPVPKGLVQGSLF | | |
| 8 | 150 | MNDKKNREPMVIALATGKGGSMKTTSAVFL | 41.6 | ['ParA-like ATPase involved |
| | | ACALVDQSRGEQRVLVADADVQGDAKDWWY | | in chromosome\Vplasmid |
| | | KAAELDDPLPFDVMSAAPADITHLHGINGR | | partitioning or cellulose |
| | | LDDPVDWILIDSAPYGRALDESVNNADLVV | | biosynthesis protein BcsQ |
| | | IPSSPSRIDLDQAAGVKDLCDRRGVPAAIL | | (ParA) (PDB:6NOO)', 'Cell |
| | | LCRTEANTTALRDALAWMDDAGIACFEALI | | cycle control, cell division, |
| | | PKRQDILNAKSTRPRGSRLHEYRDLAAELK | | chromosome |
| | | QTMRQLKDKEEDL | | partitioning!!!Cell motility'] |
| 9 | 151 | MRNMNLTPRPRDLTALLNSNRTEPADTGRP | | |
| | | ESVETEIPKNRRTVKPNDAEGWVKTSVSLR | | |
| | | ASTRRRLKTWAAEHDMRIQEVVDAALETYL | | |
| | | GLK | | |
| 10 | 152 | MFLKGECADFPDSWSDRMWGPDDLPNQRSQ | | |
| | | YDLRRAAVRICEACPVRAECLAFGIMVRDQ | | |
| | | YGIYGGLPLRARRQVLKTAQETGFRFDPDD | | |
| | | PTAERRLARYIRENPEIVAAAREKECKRRK | | |
| | | TEQRNARQQRWRTTTRSTGKAKAPAAATHT | | |
| | | PPLQDTLF | | |
| 11 | 153 | MTTNTKKVAFVNLKGGVGKTTSAIEPVETP | 47.9 | |
| | | AENITEPGKDGRKCPKWLPPLIAAVCAVIL | | |
| | | VAAGIVGWNAYSGAKLAEAKEACAAAAAVR | | |
| | | NNANEYNALLNGDAADAAAVKAEQVKDSKT | | |
| | | VESLGKELKAMAPEYEGCVAEDAQGLDAAT | | |
| | | VKLNEQADWYETHEKSLSKAVRAVTESKAA | | |
| | | KTLETAKTNLTAKLGEASKLLADSDGKVAD | | |
| | | NATRDALSNAIDAANGLKDGNDPAKIDGAR | | |
| | | KTVEDAINGVNASVQAKTDADAQAAAAAAQ | | |
| | | AQAQAQSAYNGGSSYSGGAYRRTEGSTSGS | | |
| | | NTYRGTTSGGTGSGSTSGSGPAGGSAPKPN | | |
| | | LNGSYGCGNSCTGKDDGYYHH | | |
| 12 | 154 | MNFGRKMMKAGVAAVAAIATLGAGGVVAST | 54.9 | ['Cell surface antigen C- |
| | | AFAGGGGGNQPGVGGNMDVLQFWQYKDDTS | | terminus'] |
| | | GSWGPATSLDSVRAAMNNAGVALQGDGVTK | | |
| | | AQAALDQARTECETGFRQRHPGEGDGDCRV | | |
| | | VAVGAVPYISGRNFIYNGTGYYSPSLPGGW | | |
| | | YDNWNKYVAPGTYQYGSTVYRTSYPFDDDP | | |
| | | SNSVDAIMRRNVGASSKPSIVVIVLDKYQP | | |
| | | APPNYDLTVSTQAGGTFTQAGATGNVSDAI | | |
| | | TTSRGNSSISENVTGTITLHWTGLDGTTRT | | |
| | | ASKQFTQDNNTTQNVSFGFRDVDKTWKSWP | | |
| | | AGSYYYDVNVPKQGKMKADAGHAGSADARE | | |
| | | SWKPVPTPPSKKLTNAAGQQVTSDAQQIAS | | |
| | | GSLYTAHITAQSNASEHFWLYDTIDVTAQK | | |
| | | VLIGGTDRDDVSKVTVTDQDGNAVKADITV | | |
| | | DDSQPGKRIVKAHVLNPASGQYTLNVPQSA | | |
| | | TPTGSDYTIPDDSQACWTGDEYGSTDKSHC | | |
| | | QTGNSEQVGKVTPKPDKVWVLDSNGALNAE | | |
| | | DPEHTNDKGSDNRTFVTGDAIGAVVNGRIP | | |
| | | AHLLNPFTSYSITDDWTASAQWIDWNHKDQ | | |
| | | VRVYVDGKDVTDQFDITIDTAKHTTTATAK | | |
| | | QSFLTKTAFGTADRKVKLYIGGIVKQVPNA | | |
| | | QAAADQKKLTNKATETWNNESRPTNEPPVW | | |
| | | VRNPKPDKVWSADQGQAANAEDSAWANNVN | | |
| | | ADTHTFVQQDDFGVTVNGLLPRNLARKMSS | | |
| | | YELGDDFSKSARNIDLDSASVTVTIDGKDA | | |
| | | KNLFDVHKQDDRVWVSAKQELLDTTYNQAA | | |
| | | DRKVRMTIKGAFLKDVLKAGQKVQLTNGDW | | |
| | | EQWNQQTVPGNEPPVKEWSPNPDKSWIKLG | | |
| | | DDGKWAAVVDPTGSNKTGADTLKFLDGDQV | | |
| | | ASVVNGVIASDLVKVTDIKLTDDYGQADYI | | |
| | | WDLASDQSQIRVYEEDATTDAASSVADIAN | | |
| | | KGRDVTDQFDITVAGTKVTATAKPEYRAAQ | | |
| | | AGLKNPKQISLLLPGVVNFANGKGAAQVRK | | |
| | | DFKKNAGDELTFCENPDGSKLTNKGSEKVN | | |
| | | NESQPTNEPYICGYVPPVVKKVVAEGSQGG | | |
| | | ANNDANDKVVYPGQKVEYRLTTRPQLPSDL | | |
| | | AYRIVSIRDTDTYDQYLEPDPQTLEVTDLA | | |
| | | TGDQLTTSDPQMGVEGDYTVAWDNANHQFT | | |
| | | ITYSDKYVAEHWQAGSHPQVQIRFEGTVAK | | |
| | | DAPTDRRVNNQWMLTLNNSITPSNIVDNLP | | |
| | | PKHDPSKKDNQSKEQGDPSISIDGKTMLLG | | |
| | | DTGNYVVTLDLKQTNNAYRVWKAGITDDED | | |
| | | DEHLAIDGTKIEVLDSKGQDVTGKFNIQIK | | |
| | | DGVAYVYAKTVDTWIPKKGVTVKGDPQPTD | | |
| | | LAAYASSSKHDPLSDPSIDQNLLGQEYRIV | | |
| | | MPYKVVKVEDGYTVRNKAIQVINDLTRETN | | |
| | | EVSNPLKEINPAKDVTIKVGGESIDGRSVY | | |
| | | KDRTFLYLFDSSIIPAGRAYPRVDQWRIVD | | |
| | | PLNTEYDQYTGQWAVYASRDLYRDGKVIAA | | |
| | | KGDKLAGSGFDSSKFGGDLFDAAADANGVV | | |
| | | TVEATEAYRTLVSADNSHENGWRAYIQCKR | | |
| | | LKVSDRVENRFTEYENDKEFESNIVWTRTP | | |
| | | DMTPSIHIEKYDVASGEQAGDRDDVKDALK | | |
| | | MAGDSQQIAFKITNTSKTDSSTGEGAWYLA | | |
| | | KDLKMVDRTIAGEGDVTDLKYPDNWDTLVL | | |
| | | KPGESTIITGTLKGVEQGGKHTDRVKVTGT | | |
| | | PLVECPVTDQFGGQQSTDGDQTGDTKVDGD | | |
| | | ASDTTGLKQVKVGDRTLCEDTTVESNTDDW | | |
| | | NGYRAKPLASTGTAVLGLAGGALAVLLAGG | | |
| | | SLLVFRKRHRAQGSGRHTAANAGK | | |
| 13 | 155 | MADRQTPLTECPVDTSAPFGDGTSDDESGS | | |
| | | KPEAKTESKSDDVVTIDGKDYCSDTKVESA | | |
| | | TDDWNGVAIAVARRRRKAVTAGAEHASDDA | | |
| | | Q | | |
| 14 | 156 | MVDFIVGILNSIGSGVGDDLVADLLKTPAE | 43.1 | |
| | | YNAGMYQLSLTVARSAVKPIASTILAIMCV | | |
| | | LELARVSTRADGDRELGVKLVAMAMFKLTL | | |
| | | VFTAAQHSELMLQAIDEIGDSVLGGIHSAA | | |
| | | PTTGASSGLGLGDSMRDAIDSAGVLGQIPC | | |
| | | LILLLIPFLVSKGATIVVTVVILLRFVQIY | | |
| | | MLTAFNPLPIAFIAQEETRQWGINYFKQYA | | |
| | | SLVFQCATLYLAILMYRTLVGGTLNPSKEK | | |
| | | DGDSLSGWVMDNFTGLLLASVMLIGIVMAA | | |
| | | NSVAKKLEGGE | | |
| 15 | 157 | MFGKKKTPVVAPAEGGAEARAKARRKKATR | 54.5 | ['Type IV secretory pathway, |
| | | LPKGVKQLIGYDAMLRNGIASLDDGRWSAT | | VirB4 component (VirB4)', |
| | | ILFQDINYQLSPESHQMEIIDRWAKLINSF | | 'Intracellular trafficking, |
| | | EAGQSVQIASYTRSRGVREILADVMMDETG | | secretion, and vesicular |
| | | DGLDHYRLDYNRLAQGKLESVSRNTSTVKT | | transport', 'conjugal transfer |
| | | LTVTVRESDEQAAVATLNALCNNLVSQMRS | | ATP-binding protein TraC'] |
| | | IDACKATRLDREHRLRLMAEVLRPGEEFRF | | |
| | | DERRFEHQPGKPDTKDLVCPWSIDARNPTQ | | |
| | | LDIESLDSKYLHRTMWVSSLPPELSDQLVN | | |
| | | DLTGLRARVDVSIHLAPMDRGESMTLVRRK | | |
| | | NAEVKMQIMDQRRKNRKQGLDPDDLPDDLA | | |
| | | DQQEQLGQLRDELRSTNQRLVDSIIVIGVS | | |
| | | AASQEELEVACRNVKAKVNAQSCTAESLKF | | |
| | | MQMEGLTAELPLGNNPLPMKRTLTTNSAAI | | |
| | | LIPFTTQEVFEPHGLFYGSNARSGNPILAD | | |
| | | RRSHMNSNGFVLGTSGGGKSFTVKQEIAGM | | |
| | | FLNRDDEVIVIDPEREYLALAAAFGGQIIQ | | |
| | | ISAGTGTRVNPMDIVLEDDSASDPVKDKTN | | |
| | | NVVSMIGALIGGIDGLDPLQKGLVDQCVSN | | |
| | | LYTRYRNQGGGVVQPTLQDLHDELQAGGDQ | | |
| | | VSRYLADALNPYITGSMSGFNGQTNVDLSN | | |
| | | RFTVFDVSGLSGELRTFGMMVVIDQVWNRV | | |
| | | IRNKANGRRTWLYVDEFHRFFSNQYAAAQF | | |
| | | KDIYKRARKYGLGVTGITQNVEEILDLQDA | | |
| | | REMLSNSDFLMLLSQNSTDADALCELLTLS | | |
| | | EEQRQYFTGVLPGQGLMKIGSAYVPFDGRI | | |
| | | PAGGDLYRLYSTTFQEGK | | |
| 16 | 158 | MSGTQAHHAGGTRARIGRAATGVVSSTAQF | 51.7 | ['Soluble lytic murein |
| | | GSDRTDIADSMGHTAAEMAGRAGMHGMSST | | transglycosylase or |
| | | MHGVGWTAGRARRIMNRGKRALRSGRGMRK | | regulatory protein s (may |
| | | TAGKPKALSEAKPSDEIGKFAAKGKASKRI | | contain LysM\Vinvasin |
| | | GKHIGAGLGKAGRSVKRMGSTGMGWMDEAG | | domain) (MItE) |
| | | ARLTAADDDFASKLGSTTRDLSFKAARAGV | | (PDB:153L)!!!Surface |
| | | KGVNSSAKFIWRHRRSPAKAVRGAKATGQA | | antigen (PDB:1QWY)', 'Cell |
| | | AVRAARAAANFVRMAASRVIAGAASISLPI | | wall\Vmembrane\Venvelope |
| | | MPVIAAMLAVLGVLLAVMGAFLGSSASEST | | biogenesis!!!Cell |
| | | VSGVPAEYEADVIRAGSICQVVTPSIIAAQ | | wall\Vmembrane\Venvelope |
| | | IDQESNWNPKAGSSAGAQGIAQFMPSTWAS | | biogenesis'] |
| | | AGKDGDGDGKADIWNPHDAIWSQGNYMCGL | | |
| | | ASQVETAKKSGKLTGDTLELTLAAYNAGLG | | |
| | | SVLRYGMVPPFEETINYVRRIKELAATKYT | | |
| | | ATGTAEGGTVGSLEPKLTVSGGIVSTAGIT | | |
| | | PDTRYPWGQCTWWAATRRADIGKPIPGWGN | | |
| | | AATWAGSAASAGYTVDGSPSAGSVIVFQPG | | |
| | | VLGASAGYGHVAMVEEVRGDGSILISESNA | | |
| | | LGLGVVSTREISASQLAAAGSGVRYIH | | |
| 17 | 159 | MEPKRNRIIAATGFAVALLILGGNVAVICA | | |
| | | GNGTTEDTQSTVARPRTKTEPGQKTAGDEE | | |
| | | TEPATPTEDDPCADLAPKALGVYMGDERGQ | | |
| | | LEGEYFTPDAAGLDIPASSIAPQPLPETEF | | |
| | | TGFPVSTGRRVATCAVSTGLEASWVLDYTL | | |
| | | TDDGWRCAAVKGPLEGGYRVHEGKPEEQK | | |
| 18 | 160 | MTRIDRKTGEPILSPKLTVDQLYAMANEPG | 44.0 | |
| | | WRPWMRLIAEHPHAWPELAEWWHTAQEQGF | | |
| | | DTAGAAPLPPASMRGRRRVAIPSAPLPPED | | |
| | | EPGQEPVSAPAEQSPPDDSAEKALKDADDD | | |
| | | FAALERIADLESDTADIPPIPEAESSGPAV | | |
| | | TYSADPDDLKVRRVFPVGKALVAIVMAASL | | |
| | | IAVSWMGLQIKNRRAAAMRQEAHETAISAC | | |
| | | DSAEATRKTVQSDLDRTTAKASRLLKDTSR | | |
| | | GQVAEPKTLDALNRLLDAKTSTIKGSCAPD | | |
| | | AVTSDVDRTTAALRRTTKELKNRLTDLKTA | | |
| | | TKAVTDSKLDKTVDDANALYKQTDGKVADD | | |
| | | KTRASLLDAIKKRDADAIAKAVKEVNESKM | | |
| | | AKEKADAEVKAKAEQEAAAAAAQQAQASQS | | |
| | | QSVPQRQTPSYSGGSQSQSQGSSGSGSETV | | |
| | | RRPSSGGSSSSANTGGASPGWSVPAPSDEG | | |
| | | TGLPGSDPGL | | |
| 19 | 161 | MTRTTVSGIRPDKTGTGEWIDINGHVGRLM | | |
| | | STLMADGRPESIDADPAGMTIVTPLPPPAA | | |
| | | GMETKASDGNNAKIETGSDEGRITSRKTLY | | |
| | | ERFKTRLNENLDERKQ | | |
| 20 | 162 | IMTTEANDRPEPVIWFEGTLIRDPQPHGGQD | | |
| | | DWLLETLADADGPKITIHASGEDHSANIRD | | |
| | | NAHRGSRLIVKGTAGDEGSGVDIESTSLAF | | |
| | | DPSHDEPDGEQ | | |
| 21 | 163 | MNQQEQATKSAAIFQDTINGTNDPTPWPVT | | |
| | | MWASSGDTIWTAGTARTAGEDSVGMIYGPG | | |
| | | DTIVHRNTIAGDTTRATESFAIRPADGQSP | | |
| | | MDAMLAGIEQWNHRHPDRWDTVTTPGQYRM | | |
| | | TDPTTGRPLPMGWSASLAAAASAAGRLDPD | | |
| | | LLQASLMQNAVEHEPRPCVFFLEDNGYDLM | | |
| | | VFSWHRNQQGLFDAMSFKHLQYDDLSMQVT | | |
| | | TINHSEDMSESFPAKTMSDGELLTQSRIYR | | |
| | | DEYQHWREQDGGPVAHGMTGRVMRSGLLKP | | |
| | | GLEQKPLLNLNDGRRAPDWDEFTDQAAMAI | | |
| | | LQGRPISPTPALPQQEQPTQATDPAAATQT | | |
| | | PARTAATTKQVWPNAWVANRLAHTYILRAK | | |
| | | DGRDWPKMIVGLPRGTAIDEQDLTGWATDM | | |
| | | FMSGKNQKQKNEGRAVNLRFKPDTPVELFT | | |
| | | GRGTERRTMQVDPQTLVQAIIDAQKRNRDA | | |
| | | EETLDTASVELASKTVEESWPQISRMQGRE | | |
| | | TEYQRAGTYKPAVAMKWARRLVDRTAETDE | | |
| | | ERWTNRQRRQAAGLLIQELAGTQEPTQDRD | | |
| | | KTASRPTVEQDAEATDTPRKTEPTTSTEQG | | |
| | | RDHDEAGSPALAAVTETKAESPETTRHDKP | | |
| | | ERRHTDLKSTLDRFRTRLRENLDGLNTAMP | | |
| | | APGLDQLDEPTPERGRDLDRSEPKRPAPER | | |
| | | EQPASKCHGLGL | | |
| 22 | 164 | MPIEEQAEEKIQNAVQTIITGGAKLMLRIP | | ['Protein of unknown function |
| | | KGVAMAMLRSGMKLTKTGVYAAGEAVKNKI | | (DUF3801)'] |
| | | DSGEMSEKRLQRKKDGDLHELQLDDSTMRE | | |
| | | VQHSLKTAGIDYHLERADQGQFILHFAGKD | | |
| | | EDHVRHAVQRAFKGMGLDVTDDDFTVEQTE | | |
| | | QQERTTEQTRNEPETPARETPVEPAAKPLP | | |
| | | PQRIAWDFVDPEVMEMAANSLAARHPELSW | | |
| | | DKLMGDTTWNEQTGRDFADRIIGKAAADPS | | |
| | | LRDELDAILRDDYGQGTQTQEQNRAQTPEP | | |
| | | SRETPATEPENQPEPTPAEARAQAEQQQTQ | | |
| | | ARADNPRPAGKEQKPKPIRSKKALLERFKT | | |
| | | RLNENLAEQKNHMPPTQNRDRTPRKGR | | |
| 23 | 165 | MAVVKLGKPVKSNLGGPNGAMAYIIDPAKT | 34.4 | ['Relaxase\VMobilisation |
| | | DGGRLVSSNYERTGTDYDALADPMLEDNEN | | nuclease domain', |
| | | SPKGIRKNSRLAYHIKLSFSPDDPVTPEKV | | 'Intracellular trafficking, |
| | | HELGVEFAHRITSDEYKFVVATHTDRHHLH | | secretion, and vesicular |
| | | DHIMVCAASRYGKHLKAELPKDIIDQWRAV | | transport'] |
| | | SDEICRREGLSVVFNPVVEKQTRKMRDGTT | | |
| | | AEGDDGTSPARDPKYVDEAPVRSANEPEAT | | |
| | | ARKASGGEPLERRYGMSMEEIYASAKGLGV | | |
| | | KDRLRMLIDLTSSMAENFEDWKDILDIRGV | | |
| | | DVTVRGQHLTYILKDTGFKIRDTKLGQAYD | | |
| | | MTNIMAGLQSTPVIPITENRRLVAKQTRKT | | |
| | | ITVWLPSTHRRKKITFDAKRLVDDGGSTLR | | |
| | | AFLPRDRDQIILDPSNRYAGKTPTTGLYQW | | |
| | | FGEPTSRLEPLTSPERLPLRYGVSPAQQRY | | |
| | | YQAQARRLDRLAGEAKALNAAIRWTRLADG | | |
| | | DSAKGLRLLRAKVRESHDELQAAVIALHDA | | |
| | | IQQGDPDLVAETRGEMERREALCDRYEDEL | | |
| | | SAIEREIHTTRDREQTETEQREQQQHKRGR | | |
| | | SI | | |
| 24 | 166 | MSWAAGRDRNICKRITFTQDEWEQVRNLYE | | |
| | | ELTRYAPEHRSFSSYARKMLSERRIHVTEI | | |
| | | RPLTDPEPLAKEIGRIGVNVNQIAHWANAN | | |
| | | EHITAEQVAELRASFARIERLLGDLFADKR | | |
| | | EARKDV | | |
| 25 | 167 | MTIDYSIKVTQCEPMPGYKLKVTCSDGATG | | |
| | | IFDMSRYVERGMFKPLKDTQTFDRVRLTFG | | |
| | | VPSWPGDIDIAAERVRSDMQII | | |
| 26 | 168 | MEDNESKEADMPVISMFFGIIITMNADDHV | | ['Domain of unknown |
| | | PPHIHARYQGHEASFTFDGNLFKGDFPRKQ | | function (DUF4160)'] |
| | | RKLVEAWVLLHAEELEADWDLAFNLEHPER | | |
| | | IDPLR | | |
| 27 | 169 | MEAICPAGIDLDRYATLANGNVGYDEADAH | | |
| | | WDDAAWWDEQERAMWHGGHGSLTGNGVRHR | | |
| | | CRAHPEDELLAGRLTTRQLTRSETGGFDAF | | |
| | | S | | |
| 28 | 170 | MKGDADMKEKGRAMEWERTKHDELAGTTRT | | |
| | | NFAAHGLEPGREAYHLGSIYYATAE | | |
| 29 | 171 | MQMEPMELQHVVFETDEGRVDAMWSDVRLH | | |
| | | THDIPDGWHCYAVRGDDGGWPPCSIEKSAW | | |
| | | VNHAGDIVTPDDLDPLLERNDWMLVIRDWW | | |
| | | FTDEPFE | | |
| 30 | 172 | MASDYVDDFLRDERESFKRKETAAKKEDNR | | |
| | | LDNFRKSAQALRDAARQFEEAFPGMSRTMI | | |
| | | ADKLAMTGTEKNIAFDAKGELVAAPAGRKT | | |
| | | PPAEPEPETVAESNPETTGSDVQAADDETA | | |
| | | PQEPAAEPDTSDDGRPAWASGNY | | |
| 31 | 173 | MRLIIAEKHSVGQAIAQAVGGHAEKHDGYI | | ['DNA topoisomerase IA |
| | | QVGADLVTWAQGHLVDLAAPDEYKNHDWGK | | (TopA) (PDB:1CY0)', 'DNA |
| | | WSLDTLPIDPTPDWQWKVSRDKGADRQYKV | | topoisomerase', 'Replication, |
| | | VAGLMRRDDIDMLVDACDPDREGEAIFRRI | | recombination and repair', |
| | | VAHVGVSKPMRRLWVASLEEDAIRDALTSM | | 'DNA topoisomerase III |
| | | KDETEYQGLADSAMIRAKADWLIGMNASRA | | [EC:5.6.2.1]'] |
| | | YSLVYNARFTVGRVQTPTLAMIVDRDRDIT | | |
| | | GHVARPYWKVVAPMGGWKLAGERLDKREDA | | |
| | | ETLLRIVNSDDFTFKILKADRKQQHDAPPR | | |
| | | LYDLTGLQKDMSRLHGLTAARTLAALQSLY | | |
| | | EKRLATYPRTDSQYITHDDLDTLRGLTEGD | | |
| | | RLVTGFIEPSAKPERPRLELTVDDTKVAGH | | |
| | | TAILPTMQAGKATLDELGDDERLVLIRVAR | | |
| | | RMWEAVGDDYVHDVTNVVADINPAWGERHP | | |
| | | TDGTTLDESQTRFASRSDQPVSLGWHAIEH | | |
| | | DGPQEEHDNDEAAGNIIPANLVVGVSIAPV | | |
| | | PQCGATLSEGKTKPPKPFSEATLLAAMEHA | | |
| | | SRWVEDKELKAALDDDESHSGGIGTPATRA | | |
| | | DVIEKLIHTGYVERKGKQLRSTEQGRSLID | | |
| | | VVSPKLKDVALTAGMERRLSEVEHNHADPA | | |
| | | QVETEFRDLAVRIPADARSAVRQDHVQTRT | | |
| | | RDTESFGPCPRCGKPVIKTGKVFQCSTNRR | | |
| | | EKHRRHMEDHGGMRLARLDDRGRQNRHRRD | | |
| | | HAQAARRSEGQREGLHLEEGRRIRRHAHHR | | |
| | | QRQGHRIRLQRQQGKEKEMTETQQTMTEFT | | |
| | | YATGGGRHRRRPVQPVRGPVLAQDQRRREQ | | |
| | | HAR | | |
| 32 | 174 | MVLFWLRTSVAASNMRGDVQEPEHFAGRDP | | |
| | | ARLVFADVEGLRHQESMDMAQKQYTAQTNS | | |
| | | VLIQQQMLEAEGVAVGYTYLPEETE | | |
| 33 | 175 | MFGLYMLAIYLGVAVIVAGFVALLVIVALA | | |
| | | VGLVTHPIRTLALVFHKLAALAAGLALILA | | |
| | | LVVWFWTDHAKPDFVPCFWGSIGVIVASVL | | |
| | | VRALAEWILERPTRAERRAMRRVAAPTFDS | | |
| | | QDQGGRGPSHVLSDPQQGGSAG | | |
| 34 | 176 | MAEAQEQEEPLSEAKAGKILDAVYEKILNG | | |
| | | IPLVSRSVDEIADDYLSHHDTPEAAARSLA | | |
| | | RWQVAKCGTSGELSGLGGIVTLPATIPANI | | |
| | | GSVMYVQMRMIAAIAKMGGYDVNSDQVQTL | | |
| | | VYMCLTGTTIADIVKETGIQIGAKSLTAAI | | |
| | | KKIPGAVLTKINQKLGFRFLTKFGEKGVIN | | |
| | | LGKLVPIAGGLIGGGVDIASTIVISRNAIR | | |
| | | MFIEDETLDMSEPTEEEINAAEDITVEATD | | |
| 35 | 177 | MPFLFSLLPRRRLEYGSAKYVAMPGPSRRR | | |
| | | VWRANSVPLSCVTPPRAPAGSGENITSCAP | | |
| | | TLSPAVLPGTIPAMRNLVFLSTSVCGLHPA | | |
| | | PITPSASQWPKPLRSPASGGRSEMGTRPGI | | |
| | | GKRDVLPPPRLRRLPWPRGRRRARRSRRWR | | |
| | | SAQIQRQTVSGHTRMAESSGNRTRGPPLTG | | |
| | | GGDQPRPSHSATRAHRRSHDRR | | |
| 36 | 178 | MEQIAELLKAIGEFLSGLGAALAPIAAVAV | | |
| | | ALIAKSKPRKPLNRRRKR | | |
| 37 | 179 | MNGKIGIIALMFGVVSLALAIASQSVPAGV | | |
| | | FGMCSGVLGYLAGRASNGD | | |
| 38 | 180 | MTWTQIDDGLNFSPQTMPGTVSNAALGLWV | 47.4 | |
| | | RLCVHTAYQLRFPAFDGAFDLTVVRSLKGN | | |
| | | ARQVTELEAAGMLEPALAAGRWMVVEADTL | | |
| | | MKFGGTSGSELKEKRAKAGHAGGVASGESR | | |
| | | RSKREANASKQNEASASSKPRSKTEANHEA | | |
| | | KDEANGEAKPKQTSEAKRSNCFEANEATGP | | |
| | | NLTIPSLTSPVAPSAPNAEPSQAVAESGHA | | |
| | | RPVSSLAEAEALAEADPFAFAWDRYPSHTG | | |
| | | SRDQAQNLWQAVTSGSDPTMPQAEPSQLLG | | |
| | | AVIRYAQTVRQDGDRFVPSMRKWLENRQYT | | |
| | | QWLQSVPKRTEWGGVTRQWLQTHAISQVPE | | |
| | | GSWTDSVEQTFWAHVKTGEEPETVAQRLVT | | |
| | | EINERHQA | | |
| 39 | 181 | MVNAAEYLIGFFEAGTTDEADRQGYRFPDV | | |
| | | VQALSEVESAIDSWESMGVDVHLMRSCLER | | |
| | | WKKSALNTFMDIDELRWDMSMFTHAKTQEK | | |
| | | LTDGDLMGLQSVAEKLSASTVSYSEDARQK | | |
| | | MRNMIEEAVKCVRADDSLPSDLLAYLSRLI | | |
| | | REAREALDEYELTGDFKLSVAFDRLCNALR | | |
| | | VAETKTKKHPVWEKFNEQFMVPLIAQVGVN | | |
| | | AAVYGLTVAQVLPAIGS | | |
| 40 | 182 | MSHEDINEASGDSRIQSGADSGADGQNTEI | | ['Function unknown'] |
| | | RGSENHDISRIADQGVEHDRRFDSEGDGSP | | |
| | | VVSTSLTHMEVTSGPLPSSKELAGYEQTLP | | |
| | | GAADRILRMAEDSLHSEIDSQKRIVEIYAD | | |
| | | DRKAENWVYKFTTVVFSLVPAAAFICSVVF | | |
| | | FALGMNPAAFISFAGSVGFVIPRIIEAFKS | | |
| | | GKASKNEDKPKSEN | | |
| 41 | 183 | MRSSQQQAISIRIIVQKERQIGSLQDEVDG | | |
| | | LKKILRDQSWQDHNHDICQETGNFFWLC | | |
| 42 | 184 | MTKQVRILKNDSDAGGAKPIAGELSGRLFA | | |
| | | AHMYAAAIRHRKKTRDSENRCLSGTGIALN | | |
| | | SKNFPTLQRKGYRSENEPAVFVPCITFRHV | | |
| | | VEDENAAFDVPHHNGAAPDL | | |
| 43 | 185 | MSATVAWGDESIRHKGLARPMYLMCACIID | 48.9 | |
| | | DVPAANQSMRTILPEHSKKLHWRDLTNRRK | | |
| | | RQSIDFIQQIGPLSIVIAAEESSVHKDERA | | |
| | | RRKCLERLLPVLESYEISTLVLESRTLPQD | | |
| | | QRDLEHLAQMRTRHLVRTIRMDHIRGSAEP | | |
| | | CLWIPDQVLGAYGDTKTGKLDFSEFLRENV | | |
| | | LDEIIAC | | |
| 44 | 186 | MLFAGIHPLCRVHDIPLVDISHKTGIGCVN | | |
| | | RNGIALILVARLPHKRRRTRLEGCTPEEFR | | |
| | | SMSLAVSDCIPLTDVQQTGCGSVP | | |
| 45 | 187 | MLLPCTVRYRFRLRDSAVSLVSATKKPRIL | | |
| | | QGLTDCVSLIGPAFVDGASSINAELVRQRR | | |
| | | RANNSCGYKRRSIESSHRVASFRYFLRPFD | | |
| | | LNWNVNIGLAVLPIQQGFTLWTPRRRDERP | | |
| | | RRGEERRRRPGSRTGPSLRGLGGEGDDSGQ | | |
| | | QHHDDGGREEQVIAEAVLEGVHQ | | |
| 46 | 188 | MLFLIRGNDKWPKCFAEELRLRQFIETLKL | | |
| | | LGKIILIMMESFRVEICNDSLRMLTLITSL | | |
| | | SFTKIESNGIYFFPLI | | |
| 47 | 189 | MVRPASMTVSTALTMAANPHSAATTRMSSS | | |
| | | HTRTDARKPSADGIGTIGSRPAKADRVLHA | | |
| | | ALPVAGIRVAEPGPEPVARAEQGEQTGPGG | | |
| | | RAVGVAVAHADGVVEHHDPGRHARPLEHLR | | |
| | | QTPAHAFRGLARQRRHLFVEN | | |
| 48 | 190 | MIAGFSITSLLLILLAGIGAGFVGYAVGAS | 56.9 | ['Sulfite exporter |
| | | SLVSYPALLAFGIPPVLANASNTVGVVGTG | | TauE\VSafE\VYfcA and |
| | | IGGVMGARKELKGQAVRSITYVVIGAFGGV | | related permeases, |
| | | AGAFLLLKLDPSVFEFAAPVLILLSSLIIA | | UPF0721 family (TauE) |
| | | INPRGRMQAKQAAADATAQLKHIEAADAAK | | (PUBMED:21183667;22797525)', |
| | | RARANSAASDDPAGGAGADSTAVSSADSGA | | 'Sulfite exporter |
| | | AASVAAPHRPPEQLVQPMNQDSWWVWLGVV | | TauE\SafE', 'Inorganic ion |
| | | AVAIYSGYFGAGAGTCALAVLDAAKIGPFH | | transport and metabolism', |
| | | KINALKTLIGTGANISASVVFIIQGAVDWP | | 'uncharacterized protein'] |
| | | AAIMLCIGCFIGGYIAPPITRKIPANIMRA | | |
| | | AAVIAGIVLTIDLGLKTY | | |
| 49 | 191 | MRPFSKKFCVGLVIAGIALNVLYWLAAETL | 56.7 | |
| | | HLCTGKFLLPSDFIFDYWKLPSMMIGFVMF | | |
| | | LLFEKHHFSSKVVNWIAGSAFGVYLIHYHP | | |
| | | VCHELWSTYLPVKNLIRLSHPILNGMACIA | | |
| | | GIFLCCLLLDMARHVIFALTIDKHKGAFFD | | |
| | | KLYAYAEKRKIMSILKATNTAITNL | | |
| 50 | 192 | MYGLEPQSTTDQWDYEQGLTEIIDALVKGA | | |
| | | DLIDLDYFVHVLVPYASGLEMRGLDYGERQ | | |
| | | ENIDVIRYLRDKNTILTDNTVFSRIIHLQR | | |
| | | ILAIVLGAEWTLYEASPRGQFIQSDMGFCP | | |
| | | ASDHGKRCWIVPLDPKLVILIKPKKKHCIA | | |
| | | NYREINDMQGNRSASWVVCFRRGKLNDEQM | | |
| | | LQVNENILEHSFRICIGDGLRSFKDYSYEP | | |
| | | LSEEKNNYLISIGAELENSLEVSERVEHEF | | |
| | | EWYTVSRIAAERLSPDKAKEIQWNRPYRGS | | |
| | | YPGRWRPFMPFLPENLKLSPSGLCIKDNRL | | |
| | | ILSLRETVPFSSLKCSNTFESNMAQIQELV | | |
| | | NR | | |
| 51 | 193 | MSVSSDAFHRLGRSRFQAYADFARNSGIFP | | |
| | | ATTEVDGIAMELYRWNAEASAVLMRYIPWI | | |
| | | EVLVRNAIDEQLRLWLSRQTPQPYDDWIDV | | |
| | | ADTHPMDRIRALINTAEKDYLSEARRTALS | | |
| | | KKRFWRSDQSHPRHGDEIDRDDVFAQLTFG | | |
| | | TWDGMLSRAANDPELARILMGAFPNIENAW | | |
| | | ESETRRMPNSSLPGNESDSREDRLRRELIS | | |
| | | RLKGIRVVRNRIGHDENLLRVNFPKIRHDM | | |
| | | YFVLNSLGADYPRWAFPDKAELLKRLNPVQ | | |
| | | VLEQLERKGK | | |
| 52 | 194 | METPRWMGLFGDAVMTQANKTPFGFQSPAS | | |
| | | FKFLVRFELVLDVHKAKTGHIAIIYYICLK | | |
| | | LVDCF | | |
| 53 | 195 | MAHNNLEQLNFLIRSLDSEFSDIFLHLDAK | 35.7 | ['Core-2\VI-Branching |
| | | SKINPDEIVRPVSSQLYFCDRINVYWAEYS | | enzyme'] |
| | | QVQCELNLLRLATRIGKYNYYHLISGMDFP | | |
| | | LKNQKEIHTMLRNRNDIFIHFTTHKNVELT | | |
| | | IPFVRYYHLFQKQLCVANRDHTFSIYKVFE | | |
| | | KISILIQRLIHLNRISTDIVIKKGANWFSI | | |
| | | PDDFARYVLDHQDCIKKLENNTRSPDEFFI | | |
| | | QTLAFNNPDYKKRIYRFKEDDSSESCLRHI | | |
| | | DWMRGSPYTFTIKDYDELVNCGMIFARKFD | | |
| | | LNKDRQIILKLMSHLSG | | |
| 54 | 196 | MYSTFWKVTGFKSFIKNALWATVYKRIKQQ | | ['Polysaccharide pyruvyl |
| | | AAAYEAFEHKYFRLSQDTYTSNKELTATNS | | transferase'] |
| | | KYQKFITGSDQVWNIRCMDADDAYYLNFVS | | |
| | | DENRRYAYAVSFGANNPFADSSLKDHYLNL | | |
| | | VNKFNKISVREQNAKKWVSTATGREVSLCV | | |
| | | DPTMLLSQGEWEKTVQLGSAPIIQGDYIFY | | |
| | | YCFSISQEIASFLHQVSKKTGMPVYFFEPK | | |
| | | EWALRCCWKNKIRLVKKYGPEAFLNYMKYA | | |
| | | KMVFTTSFHGTAFSTIFHKNFWYIDSGHND | | |
| | | LSKDDRAVSFLTQLKLTDRYKTISSLLQTD | | |
| | | LSLIPDYTQPDQALAKLREEAFEYIEGIVN | | |
| | | D | | |
| 55 | 197 | MTSLKKNLVYNMAYQILVILLPLITAPYVS | 27.4 | ['Membrane protein involved |
| | | RVLGAAGLGTYSYVYSISYYFGLVGMLGIT | | in the export of O-antigen |
| | | NHGSRSIALYRTDKVQTAQTFWNIYAIQFI | | and teichoic acid (RfbX)', |
| | | STGIAVFSYTIFAFVLFNGNKIVAYINILF | | 'Polysaccharide biosynthesis |
| | | VISYLLDINWLFFGLEQFKITVIRNTIIKV | | protein', 'Cell |
| | | ATACSIFIFVKDRNDLWLYTLIMALGASLS | | wall\Vmembrane\Venvelope |
| | | QIYLWMNVHKFVSFCKPEWSEVKKNVKPVI | | biogenesis'] |
| | | ILFIPAIAYSIYKVLDKIMLGAMSDMTQVG | | |
| | | LEDNAERIVNIPVSLITAFGTVMLPRISNL | | |
| | | IAAGDSNQIKKYNAISFRYFTMLVCGAAFG | | |
| | | LAGISNILAPVYFGDEFKGSSPIIFGLSES | | |
| | | LIFMIWANIIRTQYLIPNKKDMPYVISTLF | | |
| | | GAAINISVNLLLIPHLQAVGTMIGTILAEL | | |
| | | SVFCVQYLFTRKDFPTLQYLKSGIMFFPIG | | |
| | | ALMGIIVWIVGQILGPTIITLIIQIILGAF | | |
| | | IYGIGSLIYLIAIKDDIFISMFKKTFHINA | | |
| | | NSRLPKHRSV | | |
| 56 | 198 | MKLSKNLQILFIALAIIPFFQSTIGYKYPI | | |
| | | LGLGVQYSRYFFFGIIIILILINTHAFSFI | | |
| | | TRNTKIYLTISLIFIFFISAVVNNTNIVSV | | |
| | | FQLSLYLLYPFLLFSVWADSSTDTLNICKG | | |
| | | ITYGLNTLVLINLSIMLFFPQGLYQTISSN | | |
| | | TISYYYLFGAKNQMVAPIMTCLFFNMETAY | | |
| | | RSYNKIITKTSLFMCFICAFELIIGGSGTG | | |
| | | LIVLAAFIILCLLELKHQKISTNLSLIVLL | | |
| | | ASFLAIVIFRIQNIFSFLIVDILHKSLTLS | | |
| | | DRTYIWDAAIESILSHPILGTGITDSLSGN | | |
| | | VHLKLSYLVKDIFAHDLYLDYLLMGGIPAL | | |
| | | CIFVCILISVKKSYDSFLNNKNTLIWWGIV | | |
| | | VYLFASIVEIYTTNFCLFLMFAYINICDYS | | |
| | | TRHRYIGQN | | |
| 57 | 199 | MSLVTIILPAYNAEKTIARAIQSARCQEYS | 39.0 | ['Glycosyltransferase |
| | | DLEIIVIDDGSSDSTLNICNALADKDNRLK | | involved in cell wall |
| | | IWHINNSGPSAARNYGLSQAKGEFICFMDA | | bisynthesis (WcaA) |
| | | DDEMSPTMISQLVSNMQDDTDLVACGYKVR | | (PDB:5MLZ)', 'Glycosyl |
| | | SNNGDYAFEQKLDDKSYPNNRLYEFIENLQ | | transferase family 2', 'Cell |
| | | EAKAFNPLWNKIFRRSIINDNNIKMDTTVD | | wall\Vmembrane\Venvelope |
| | | MGEDYLFVVDYCEKSEKDFKALSSSLYIYE | | biogenesis'] |
| | | LSNNGLQVSANKNNNLKRRLSQLDKLENLF | | |
| | | KRRNYPMNAIYKEKLRIIYTSLLESNDLHN | | |
| | | DLIEVLKNGYLDDLIHSNINLGKKYTVFLE | | |
| | | ILKSRHIGVAYLSMNIFRAVKKIQGKSENW | | |
| | | G | | |
| 58 | 200 | MSFSPDITNFDKLIVLVNTGIKFIRGLWMR | 35.4 | ['Acetyltransferase, |
| | | LFLREAHGLLLIGRNVTVSHAHNVRCGKNV | | isoleucine patch superfamily |
| | | KFEDYAEIQGLCSDGLNFGDNVTIGRATMI | | (WbbJ)', 'Bacterial |
| | | RPSSYYGGDCGQGIAIGDNSSIGPHGYIGC | | transferase hexapeptide (six |
| | | SGPIRIGNNVMIGPKCSLFAENHVESNSDE | | repeats)', 'General function |
| | | SIKSQGVQQRGITIGNDCWIGSNVIILDGV | | prediction only', 'maltose O- |
| | | SIGSHVVIGAGTLITKDVPDYSIVLDRRNR | | acetyltransferase |
| | | ILRQRIKKIED | | [EC:2.3.1.79]'] |
| 59 | 201 | MGGGPLEDDFKAWADKKPNVYFLGYTPHEQ | | ['Cell |
| | | CMAIAKAGEFVVFPSIWYEGCSMVEIESES | | wall\Vmembrane\Venvelope |
| | | LGLPLIATDLGFSAEAVNDGVNGVKVPLGD | | biogenesis'] |
| | | MHGWITAIRGLWKEPELCKKMGMNSRRDYE | | |
| | | DKYTPENNYQQLINIYQSTLERG | | |
| 60 | 202 | MLRRIWPLNVYQGYLSGMQFFMVALVLRKA | | |
| | | FVYFNAYGMRLTTFRWW | | |
| 61 | 203 | MSGVSAVQHVFVVGSKGIPGSYGGYETFVD | | ['Glycosyltransferase |
| | | RLTEYHEGEPGLRYHVACKSLSESGEFSYH | | involved in cell wall |
| | | NARCFRVRVPQIGPAQAIWYDVAALADCVA | | bisynthesis (RfaB) |
| | | YIRRNRIPHPIVYVLACRIGPFTAHFASAV | | (PDB:2IV7)', 'Domain of |
| | | HRLGGKLYVNPDGHEWMRAKWSAPVRRYWK | | unknown function |
| | | VSERMMTKHADLMVCDSENMEKYIREEYAR | | (DUF1972)', 'Cell |
| | | YRPQTTFIAYGAESRRSRLADDDPKLLDWF | | wall\Vmembrane\Venvelope |
| | | RSKGLEPKSYYLVVGRFVPENNYETMIREF | | biogenesis', |
| | | MASNSKRDFALVTNVSDKFLEELKEKTHED | | 'rhamnosyltransferase |
| | | RDPRIKFVGTVYDKELLMKIREEAYAYFHG | | [EC:2.4.1.-]'] |
| | | HEVGGTNPSLLEALASTDLNLLLGVGENRE | | |
| | | VAQDAALYWSKEPGDLAALIDHADAMSPDE | | |
| | | FHALGARAKQRIADAYSWQHIADQYKNLFL | | |
| | | GREQKEG | | |
| 62 | 204 | MIHNYRSNNMEKPGIIDSRESGIRELCFQC | | |
| | | LPCIMSVTSATSGALADGEQGDARGGKQRN | | |
| | | GHTAAIADSTVIIFFFITSNLHNRTG | | |
| 63 | 205 | MMIWLSFFVAILATVFILYAPGFLLMKAAR | | ['Family of unknown function |
| | | QTSINALAFAPPLTLAIYAIVEIVLSKAGV | | (DUF6541)'] |
| | | PGNWYTIFLPFLIVAILLFIFSICMRHKEK | | |
| | | RSIRCALTNLSDTFPYWMMVVLYVAVGSVV | | |
| | | SLIVFIRHMHGPNSYTQLYDNAWHMGIIQK | | |
| | | FLASGDFSTLNAGDIVATSGSTFYPTGWHS | | |
| | | LVALVASMTGFSVPVCINASIFVILSIVYP | | |
| | | ISMFVLVGKLSAYNNNVIVAGAFTALMFAA | | |
| | | FPWRFLTFGPLYSNLLSFSIIPLVITVALN | | |
| | | MVEAKRGVKERIFLVSVFVLSTIGVAVTQP | | |
| | | NAVFTMGLLVAPYIFLQIPGYLEYAGITKH | | |
| | | RNIAVAGACVALMMAIAGVWSLIYNAKFMQ | | |
| | | RTVTWQWPSYEKKIQAFIDIAFVGERNAEP | | |
| | | QILLGVLVLIGMVYTVFHKRLLWITCGYII | | |
| | | MCGFYAVSSSTEGFLKNVLTGFWYHDQYRL | | |
| | | GASAVFYGAVLAAMGLWNLMIVIVRLVPEN | | |
| | | LIGASNSRNKCILSTFCVLLIVLVNYFPSF | | |
| | | YLSGRGDYVTAFGAITRDISYWNSPSEPKS | | |
| | | YTATEAAFVEKVKKVVPKEAIILNQPYDGS | | |
| | | AYAWGQNGLNVYYKAWEGNWMGAPSDDNSL | | |
| | | ISESLDKIASNREVKEAVRRVGAKYLLVLD | | |
| | | KSDFSKDKNDVSMMKSIYASYPERKWSGID | | |
| | | EVSDATEGFKVVLSQGAMRLYRITV | | |
| 64 | 206 | MFKLRDMKWFNAGPRKSRARRHYLVSTAGQ | 30.5 | ['Polysaccharide pyruvyl |
| | | PNWGDEFITRAWIRFLAKSDPDSEIWLDCP | | transferase family protein |
| | | NPSHSSLLLKDEHPHLHVVNTLWQLVWNTS | | WcaK (colanic acid |
| | | DLLDDSEAAAQKIRGWIRDGGTPREDFGID | | biosynthesis) (WcaK)', 'Cell |
| | | LLRSMDTVHLLGGGYINQLWKANVLLLVAL | | wall\membrane\Venvelope |
| | | AQLKELNPKVLLYGTGLGLAPLKGIDLFLA | | biogenesis'] |
| | | RTSLTAFDHMSVRDSRSADIAGTERGEDDA | | |
| | | FLEIANTGSDWLEQNASARAFVCLQQDVVG | | |
| | | RNPKAVKAAVDSLLLSGVEKSEPIFLVEAI | | |
| | | PPEDSWSLDLFKDQWPGEVWLLPFSHLWNY | | |
| | | GFPNAADTVWISSRFHMHLLGACAGARGIA | | |
| | | MGFGNEYYDIKHGSLMDLGTGWANLDVSVD | | |
| | | HPAPVSATASSKFPAESLRIARKKKREANQ | | |
| | | LY | | |
| 65 | 207 | MRHFFHIDEEGARLIEKLSERQRRLTLGAV | | ['Glycosyltransferase |
| | | DYNALIQPGRTLTYKLYMGQKWQYPRLTDT | | involved in cell wall |
| | | STLNGVLTEQAGLEPFAVFQATSSVKEKSY | | bisynthesis (RfaB) |
| | | FSPHYFAGDEQLVDRNIRCLLLTPDDATDD | | (PDB:2IV7)', 'Cell |
| | | DVEQNRPVDRHARLYRKVESEAPAFFDMLR | | wall\membrane\Venvelope |
| | | EAKRVQRERIGKTAIAPFEPATITRVNESG | | biogenesis'] |
| | | YSHHPWVEIVPGKAAPDAPKAVIIAMHWLQ | | |
| | | SGGAERWAMETVTLARQAGLLPIIITDSDG | | |
| | | HQPWITRHEFDDAVLLPLTLPLQDRVGDAT | | |
| | | LLRALFEQFNIVGVLIHHCQWMYDNVWWVK | | |
| | | RYFPETHVVDSLHIVEYIFHGGYPSESVAR | | |
| | | DKWIDIHHVISPQLEHWMEDVHHIDPKKVV | | |
| | | DAPLVGLTADSKTPTFKSRDISKPLTISFV | | |
| | | GRNVRQKRPETFILAVRELNKKFPGKLRFI | | |
| | | MQGNGDMDEFVDRLIERYGLGTVIQRCSMS | | |
| | | TPVSQTYAQTDILLVSSMNEGITLTTIEAV | | |
| | | SAGIPVISSNVGSQDTLVPPQGLCRRGSAQ | | |
| | | FVHDATAIVGRVLESEENRKKLWEAEKDRL | | |
| | | EKFSELQSANEYFRNMLTEWSH | | |
| 66 | 208 | MQNTKIAAVVVTFNRLEKLKKVLSSLEAQT | 27.8 | ['Glycosyltransferase, GT2 |
| | | RLPDQLVIVNNAATDGTDSFLKEYAANFKY | | family (WcaE) (PDB:2Z86)', |
| | | SDSVQLDIVTLEKNEGGAGGFSAGMRRAYE | | 'Glycosyl transferase family |
| | | IGCDYAWIFDDDGYPEPDALDKLFKGYGDA | | 2', 'Carbohydrate transport |
| | | VAELGPDVPFACSLVKFIDGTISEMNNPIP | | and metabolism', |
| | | TWDWGRLKAKGLDIVLVSRCSFVSVLIPRW | | 'rhamnopyranosyl-N- |
| | | VMEAFGLPYKEYFIWFDDAEYTLRITRACP | | acetylglucosaminyl- |
| | | GVQVLDSVVLHDMGVNRGVNFGMINEKNVW | | diphospho-decaprenol beta- |
| | | KFLYGARNEASYHLHHEGLYPYLRFCAMVR | | 1,3\V1,4- |
| | | NNMRQGNVDKKLQRQVYGKLLEARSENPQI | | galactofuranosyltransferase |
| | | DFPQTPISKSAH | | [EC:2.4.1.287]'] |
| 67 | 209 | MDRSRGYSLTQHPDPELYCLGHLVEEFDFH | | ['Carbohydrate transport and |
| | | AGAKGIDEKGHGEVEDVTRRMVRLEVPAWR | | metabolism'] |
| | | EPVTATLIPYFAWANRGENEMTVWLRG | | |
| 68 | 210 | MGESVRDVLFLIVLFAANVIQAITGFAGTV | | ['Sulfite exporter |
| | | LAMPFSMLLLGTNTAKVVLNITTLLACLWL | | TauE\SafE\VYfcA and |
| | | GVQHRAHIRWRILSEMVGLMAIGMAVGVAL | | related permeases, |
| | | YAVLPLAPLQKAYGVFIIVIALKNLIWPSH | | UPF0721 family (TauE) |
| | | GEPPYWLLAIIVLLAGVIHGMFISGGALLV | | (PUBMED:21183667;22797525)', |
| | | IYAAVRLKDKDEFRATMACVWVALNSVLAV | | 'Sulfite exporter |
| | | QQGVSGVMTPHALVLSAVSIPPLIVAIIIG | | TauEWSafE', 'Inorganic ion |
| | | NRLQQHVSQQAFLKLTYVLLVISGASIVL | | transport and metabolism'] |
| 69 | 211 | MTVRLKELVEQAADKDMVLVAGKGGLERPV | | ['DNA-binding transcriptional |
| | | RWVHMVENEEIAGFLEGQEIAFTTGIGSET | | regulator, PucRIVPutR family |
| | | QEDLYPLVKSAYASGATGVVVNIGPFIHQI | | (PucR) |
| | | SPDTIRFCDEHDFPLFKVPWSVHMAQIMHS | | (PUBMED:14990804)', |
| | | FSLAITMSEKHSMELAAALKNAIFHPDREE | | 'Purine catabolismoregulatory |
| | | MYLEYLEQSGFGKDWNYCVTVFSACADAQQ | | protein-like family', |
| | | GGHDAERIAKYSREAESLITRNQWRVAVAH | | 'Transcription', 'PucR family |
| | | IEERLVLVFARYTAEQVELMVREIIAAIRN | | transcriptional regulator, |
| | | RGVLLDRTYIGVGKVTKSARCIGKSYNQAL | | proline-responsive |
| | | KLERLQHLRGRVGEVALYDNSGIDKLLLAV | | transcriptional activator'] |
| | | SDRAILEDYYQDSIGALVEYDRVNGTDLTD | | |
| | | TLQAYFRFSGSVKETAASMFVHRNTVSYKL | | |
| | | NKIEDILGVSLSDERTREFLSVGLQVREIL | | |
| | | DC | | |
| 70 | 212 | MSESLATKELTGEEVAELHREYVMQSWHKQ | | ['Biotin biosynthesis', |
| | | GEPVMPVKYAKGIYVYDYGGNKYADMSSLL | | 'Adenosylmethionine-8- |
| | | VCSNLGHELPEIVDAIKAQADKMCFMAPAY | | amino-7-oxononanoate |
| | | ASEPKSKLAKMIVDLAGNDFYQRVFFTNGG | | aminotransferase (BioA) |
| | | ADSNENAIKMARMVTGRQKIFSCYRSYHGS | | (PDB: 1MLY)', |
| | | TLGASFASGDWRRFAVEAGGAAPSFVHEMN | | 'Aminotransferase class-III', |
| | | PNMYEDGENRGEDDEKVTALYLKRLEDQLI | | 'Coenzyme transport and |
| | | YENPDDVAAILMESIVGANGVILPPKGYME | | metabolism', 'beta-alanine--- |
| | | GVRALCDKYGILMICDEVMAGWCRTGKMFA | | 2-oxoglutarate transaminase |
| | | WQNFDIKPDIFTFAKGVTCGYVPLGGVVVS | | [EC:2.6.1.120]'] |
| | | KRISDYFTDHVLQCGLTYSGHTLACAAGVA | | |
| | | AVNYYVEHDIAGHVKEMEGILKPFLEDMQA | | |
| | | KHKCVGESRCVGLFAAMTIVKNKETRELMS | | |
| | | PYHTANPVMGKIMGALKDKGFLTFGRETNV | | |
| | | NICPPLTITADELKAELPKVDEVLTWVDEN | | |
| | | FCD | | |
| 71 | 213 | MLWDYQQPVAIREGNGRVREIKDVAAEMGL | 27.8 | ['Alcohol dehydrogenase, |
| | | TEGGLLVSEKLFATNGTAEKIVKDSEGTIS | | class IV (EutG) |
| | | EIFSDFSPNPDVTEVDKAAALIREKHLKFV | | (PDB:6AHC)', 'Iron- |
| | | VAMGGGSAMDLAKSAASIAFTNDSIADYHG | | containing alcohol |
| | | TGKAMPQEHLPIIAVPTTAGTGSEVTCVSV | | dehydrogenase', 'Energy |
| | | LTNRALGKKAPIVSDGFFPSVAIIDPELTY | | production and conversion', |
| | | SVPPHVTASTGMDVLSQAIEGYWSKGHQPI | | 'sulfoacetaldehyde reductase |
| | | CDACAIHAAPLVFKYLPIAVAEPDNAEARQ | | (NADH) [EC:1.1.1.433]'] |
| | | KMCEASVIAGLAFTLPKTTSSHACSFPLTN | | |
| | | IYGIPHGEACGLTLDWFARVNADAQHGRVQ | | |
| | | EFARAIGFKNVDAMADAIQELKVKVGLRTG | | |
| | | LKDLNLNAEQIADLVRISRHPNLYNNPVEI | | |
| | | ADDMLQDMYEHLAATD | | |
| 72 | 214 | MATLYTGGTIRTMSAPDATAEALLTDDRGH | | ['Predicted amidohydrolase |
| | | IAFVGSLAEARDRAESMIAVGGRGVQERDL | | YtcJ (YtcJ) (PDB:3ICJ)', |
| | | AGACLMPGFIDSHSHFAGMGQRLTDADLAG | | 'Amidohydrolase family', |
| | | CADFDEIAARLKAFLAAHPLPAGGVLHGEN | | 'General function prediction |
| | | YDHNELAEGVHPDRRVLDAALGDVPTVIAH | | only'] |
| | | VSGHVIICNTALLKLVGLNRDAEAPEGGVY | | |
| | | GRDENDDLTGYFAETPAIMPILMHSAVMPR | | |
| | | QTLGDLAEAIQNDYASHGITTCQDGATAPG | | |
| | | YAEQFVKLAEAHQLKIDVTCYPMMGQDLDA | | |
| | | VFESVGSYASGGTSEGIQYSNHLRFGGVKL | | |
| | | FVDGSPQARGAWLSEPYMPLPTSVHSNEPA | | |
| | | GYHGAGIVSDEAMRAILDHALERGWKVMAH | | |
| | | CNGDQASEQFLTQYTAAYHASSRHNKIELR | | |
| | | PVMVHCQLTRHDQYARMAAVHMIPSIFVSH | | |
| | | CWFWGDAHIKNLGFARASRISAVHDALDYN | | |
| | | LPFTFHTDSPIVPPDLIFAAWCAMTRITKQ | | |
| | | GVELDPAQRIGAWDAFRAITRNAAYQYGEE | | |
| | | ASKGTLEAGKLADLTILDADTLAASADSTE | | |
| | | AAARVRALNVLETIKEGTTVWTA | | |
| 73 | 215 | MGKTSTTKSASSAASGTTGAVVRPFGWRDK | | ['Na+\Vmelibiose symporter |
| | | IGYMCGDFGTDFMFVFAGTWFMIFYTKVMG | | or related transporter (MelB) |
| | | VPGTIVGTLFLLARVLDAVMDVTVGVVVDR | | (PDB:4M64)', 'MFS\Vsugar |
| | | SKDHPGGKFRVTMMRFTAPLVILSFLMYQT | | transport protein', |
| | | FAIDAAMWVKIVYMSITYVVWCFFYSCVNI | | 'Carbohydrate transport and |
| | | PFGSLASTISAEADDRTSLSTMRSMGGTIG | | metabolism', |
| | | GLILGIVAPIVIYQKVNGHQVIRGGDGTHI | | 'glycoside\Vpentoside\ |
| | | FAIVAAVVSIAAFIGYLICYFNVTERVHPD | | Vhexuronide:cation symporter, GPH |
| | | PDQNVKEGDKRSPLAMITNAFSSRSMLGIV | | family'] |
| | | AAALCLLVAQLFSNQLMTYVYTDVFGSAAL | | |
| | | ASISGLLGTVVMFLVVPFVKPLTKRFGRKT | | |
| | | ICTFASALGAISMLLLFVVQTRNGMVELLG | | |
| | | AVFMYFALMGFNLVIWAMIADVIDDIEVTR | | |
| | | DTREEATCFSCYSFARKIGQAVAGELAGLS | | |
| | | LDWVGYQSGATAVQSAATKTALYNVATLVP | | |
| | | GLFFLFTFLCLMVIYPLSRDRVLYNVEVLR | | |
| | | VKRKGLTGEEARLLTKYEGITPTGTALEGS | | |
| | | TIEWPDFHELERARELSARTGVPIRDPQKT | | |
| | | DDAQQ | | |
| 74 | 216 | MSDATTPTPNTNDAENTPVVAGTAVAGTSI | | ['ABC-type |
| | | VKNEANAKRKPMPAWMKSKKIQPIISLVLL | | nitrate\Vsulfonate\Vbicarbonate |
| | | IAVWWGVTSAGLINSLYLPGPKAVWDAFIE | | transport system, |
| | | ANSCRSASAGSSRIVCGEQQYFLWQHLVVS | | permease component |
| | | LERIGIGMALGIVFGVLVGFILAEIGWLNR | | (TauC)', 'Binding-protein- |
| | | IILPYINFIRALPPLGYIGLLIVWFGIGDT | | dependent transport system |
| | | TKIWLLFLAAFPPIVLATVDGINGVNRDRI | | inner membrane component', |
| | | NSALSLGASRPKAFLFVVFPSALPSIMNGI | | 'Inorganic ion transport and |
| | | RLAVGFAWTTVVAAELNNGIPGIGALAYLS | | metabolism', 'taurine |
| | | GTELNTPLTITCIFVIGIAALLLDWLILGI | | transport system permease |
| | | THLVTPWVGKE | | protein'] |
| 75 | 217 | MNETLFAKRETHRETIRKVGALLVSATLLT | | ['ABC-type taurine transport |
| | | SLAGCAKMPTADELVGNTSGAAVADCPVPS | | system, periplasmic |
| | | AKNEDFAGTIRIAWQAIPNADLIVKDKGLL | | component (TauA) |
| | | EACLPNATIQWSQFNSGGDVIQAFGSNSLD | | (PDB:6SSY)', 'Inorganic ion |
| | | IGLAGSSPAVRAASAPLNLPVKVIWIHDII | | transport and metabolism', |
| | | GDAESLVAHGGDYTSIKDLAGKNIAVPFGS | | 'taurine transport system |
| | | TSHFSLLSALRNAGMNETSVNLINLDPDKM | | substrate-binding protein'] |
| | | SAAWTRGEIDAAWVWDPVLSKLKADGGAIV | | |
| | | TSSAATAKTGSATYDMELATDSFIKANPKV | | |
| | | METWAAVENYAAGLISSKENDSAESISTIL | | |
| | | GNSVADVKKQFAGYTYPQAKDQSDIFHGQL | | |
| | | PSIFKDTAEFLKTQGSLDKVSSDYSSVLYT | | |
| | | DAIDQVAKQ | | |
| 76 | 218 | MAGNNVLTWGELTEKKTMDDQGVAVDIRGV | 41.4 | ['ABC-type |
| | | NKRFITQQGDQVTALHDINLTINKHDFICV | | nitrate\Vsulfonate\Vbicarbonate |
| | | VGRSGCGKTTLLNMLAGFEKPSDGELISGG | | transport system, ATPase |
| | | VAINGPSPKRGVVFQKPPLYPWLTVRKNVE | | component (TauB)', 'ABC |
| | | FGMKMQGVPAGERKERADHFLDLVGLTSAA | | transporter', 'Inorganic ion |
| | | DRRPYELSGGMQQRAQIARVLATEPDVILM | | transport and metabolism', |
| | | DEPYGALDALTREKLQNELLRIWRERHSTV | | 'NitTWTauT family transport |
| | | FFITHSVDEAIFLATRVIVMSAHPGTVKMD | | system ATP-binding protein'] |
| | | IPITLPRDPDDPDNMEKVRAMPEFVKLREE | | |
| | | ITQAIYVQDDQE | | |
| 77 | 219 | MQIADTNTYSAANFHCRLTAATENDEKPTT | | |
| | | INRIQMKSCMIFRFASIRIAHLIPSYSICN | | |
| | | QNAEQ | | |
| 78 | 220 | MSIDEENLDSSSLGAAGESAVSYVFTRFGW | 24.3 | ['Domain of unknown |
| | | SVSKPNPDRGTDLEVTPGDRHFPLGVQVKT | | function (DUF4365)'] |
| | | GKSFFQKREVDEDGKLIGWWYRSSNKKHRL | | |
| | | QWTTGAPVLLVLFDDKQEVGYWTYVSESEI | | |
| | | SDAGSSWRILVPIEQVLDKFHLSAIQQILD | | |
| | | DYYKRLPVTETSWSNNLKDIPKEDRMRYAL | | |
| | | LTPRIIAPHPNNYRDLSGIEILAVHVLMRD | | |
| | | ELDRAWERDEDQEAPQNRLFPIEKSFLQAS | | |
| | | ESDEWSWNAAAAVHKYLCRNDSSLIMGLED | | |
| | | KAQAQYEKVAAAILASVVCTDNDDLKKAHD | | |
| | | WICCARSTHNTKMDEAWLDVQEARIYLSMG | | |
| | | DYESRVEASHKAYAAYIFVKTVKSDRTAEA | | |
| | | LLASCSRLLWQTNNPLFIPDEDRQQYIEKN | | |
| | | PANSLMLEDHINAIDNAPQWWQGEYIGSAL | | |
| | | SKQVDFEFKGGAKNQGSPVNVDMKRKLVSA | | |
| | | AFIASCAGNLVDWQQAWRLIAITDYSAALK | | |
| | | NRDESLMLSSLGLLRSFGRMNEMKNATIKA | | |
| | | LHICSGQSFAEDADSIDLSKVLETDLNNTL | | |
| | | DYLCSIAAATHQETAKRNVTWCKRWIDDTQ | | |
| | | ILSAKSGDFSRGQRVIRLLFASCPAAGEEA | | |
| | | MRETALWAYSQPAVTNNVVAGPFANGVRSL | | |
| | | PSTVWKTIAEKRNLANDVSPVQEAFAAVTD | | |
| | | IQADAKHSHLMNGEIDYLADLDLEHKLSED | | |
| | | EACAVTKKLTASVSKTIAMAKKGVHARGGL | | |
| | | QVEVALFLIGRLHPSLRNDSLLMDELDEQT | | |
| | | LFHDEKEPLLNALFWRNDLLLDEDRQEWVK | | |
| | | HINPLAEVPPTKDGFFGAQEDIHPTAMKAL | | |
| | | AANVGKEKRSELIDQMLLRGEEFTNSAFDV | | |
| | | LSLFPDPRYITFALFTVTNSVEDALLSACR | | |
| | | FLTVCAYQGLCNSQTAEHITTLARDGSYKV | | |
| | | QCMICSTICGQLETNAVQGNYAKKLIAIAE | | |
| | | NCPSASLRWRLSHVGE | | |
| 79 | 221 | MSDIQHIGGRTPDLTEENVDKLVALFPDVL | 42.6 | ['Adenine specific DNA |
| | | TEVPDQQTGETKKAIDFDALRDKLGDVAEN | | methylase Mod (Mod) |
| | | TRERYQFTWPGKRAAKAEARKPIAKTLRPV | | (PDB:4ZCF)', 'Replication, |
| | | KERSKDWDTTQNLYIEGDNLDALKILRETY | | recombination and repair', |
| | | AGKIKLIYIDPPYNTGHDFIYKDNFGKTIA | | 'adenine-specific DNA- |
| | | ADKAENGDYDDDGGQLVINPESNGRFHSDW | | methyltransferase |
| | | CSMIYPRLLLARDLLTSDGVIFISIDDNKE | | [EC:2.1.1.72]'] |
| | | SDLQNLCNEIFGERNFVAKLYVKVNPCGRN | | |
| | | LDMFARSNIPTTRSASARRSGAACVCV | | |
| 80 | 222 | MGRIIPARAGQTSWTGGRRFSCSDHPRACG | 52.3 | |
| | | ANRRDGGAAGDRAGSSPRVRGKPRRRNPNH | | |
| | | RHHRIIPARAGQTTLRFHSTFRSSDHPRAC | | |
| | | GANRTATTNEDVVIGSSPRVRGKRLKETQT | | |
| | | EHIHRIIPARAGQTTCPNCRRRACPDHPRA | | |
| | | CGANTSEALALPLTVGSSPRVRGKQVRRRG | | |
| | | SAIPCRIIPARAGQTDSLDPGSMFQPDHPR | | |
| | | ACGANWANMGAADANAGSSPRVRGKHRGWF | | |
| | | WHTG | | |
| 81 | 223 | MRGKRVLLVETVEHIRIIPARAGQTALRDR | 54.4 | |
| | | EKPETPDHPRACGANRFFPSAVVCAFGSSP | | |
| | | RVRGKQRLTGRFRATVRIIPARAGQTASEC | | |
| | | CSRCRPPDHPRACGANLLALKAVLIVSGSS | | |
| | | PRVRGKLAREPVLAYVDRIIPARAGQTRTV | | |
| | | QAGIVVRADHPRACGANFKLSFALTVTSGS | | |
| | | SPRVRGKHHGLLSAHARRRIIPARAGQTRH | | |
| | | PIWFLRRRPDHPRACGANQPKTASTRPKPG | | |
| | | SSPRVRGKLVLQPLTQLIGRFIPARAGQTP | | |
| | | TNTGAPSSNTDHPRACGANVRVLSSSVVQH | | |
| | | GSSPRVRGKPGYRLADS | | |
| 82 | 224 | MRIIPARAGQTWRTGPRCRRWPDHPRACGA | 55.6 | |
| | | NTVLIPSAIFWHGSSPRVRGKLSQRYFVAG | | |
| | | VKRIIPARAGQTRGHAVPAMRAPDHPRACG | | |
| | | ANHLLADIADNTAGSSPRVRGKPGWGRKRK | | |
| | | AYLRIIPARAGQTGPPPGEAGEGPDHPRAC | | |
| | | GANLVSSLTGTSLAGSSPRVRGKRAGAR | | |
| 83 | 225 | MNEGVISRMARVEVTKKLRAAYRVGSKKEK | | |
| | | SAVLDRFCEIMGLSPSSARQYLMDETIGNP | | |
| | | KVLRLD | | |
| 84 | 226 | MMMRDSDRPWLRRDTIFASAPGGVLISNAT | | |
| | | TGCEIAGESAYELFSRVFPLENGQATVGEI | | |
| | | KGAVAERNWKLIEAIAAPLEEKGFLRWIPE | | |
| | | SDYELLDNEKREKYADQIAFLAQFTDAPHE | | |
| | | AFLAFNQAQILVVGSDEVADSLQANLIDNG | | |
| | | AELVTSAESFAVDQFEELAPDLTVLGPTAL | | |
| | | SGIDHLRKAGVPFLGVCPAGDYLWALPVGW | | |
| | | TEGSASWHSADSSLRRGSMGKEWAEAIEQA | | |
| | | QAGQPQWTSATSSQAVQRLFGALLAYEVFK | | |
| | | GITGAITPETSEKILAFNALTGATSTHPMT | | |
| | | PIYSEVSREVHAQLAGAHPGDAGSETPASI | | |
| | | HVSHADEYDDVWAPLVDRFTMPAFDEDDLD | | |
| | | IDQVPVKVSLVETATGKVFAASPWTTADAR | | |
| | | IEALARAYGQSLSWHCTWASDAPRVVGIGT | | |
| | | TRADAIRRGVEATVRREMLSGSGLTEPVQS | | |
| | | VLGGRLGTFVSDVAEGSLEFFAHDPLAGQH | | |
| | | VAIACCDEYRAVGAGSSQEEAQARAAIEVL | | |
| | | GRRQVGLVDTGDAQPIGGEVALSTACIGQW | | |
| | | HVAVVAPAGSRACADDSALEAVR | | |
| 85 | 227 | MIDTIQGWRHAVLPGSDTCIRCMAEQIAMT | | ['Ribosomal protein S12 |
| | | DRFFMRTGRQLIATSPRHDNPFFKGCVEAG | | methylthiotransferase |
| | | GRLGLAHGISIDRASLQVRSAATVCRHVRS | | accessory factor YcaO |
| | | EKMTTEAGGYRARTLSSYFHVAPVERYVSP | | (YcaO) (PDB:4BS9)', 'YcaO |
| | | VHAVIGAEIDFDVTGLVTAKASGFLILYVG | | cyclodehydratase, ATP-ad |
| | | DDYYTRPWGGHCGKLSDALYLGVLEGVERL | | Mg2+-binding', 'Translation, |
| | | SGEADHDAQVAVQPPANTRLIDLDAFGVPR | | ribosomal structure and |
| | | DCWKIPHPKVGAWVKASVITSGPDGAFLLG | | biogenesis', 'ribosomal |
| | | ETVMIPERLAYYRAAHDHEPWVQDSSNGCA | | protein S12 |
| | | VGGSDEEAILFGMLEAIERDAFMIAWYGNL | | methylthiotransferase |
| | | DLSPIDPASIHDEESCAYLRRMELCGVDVI | | accessory factor'] |
| | | FLDATVGVGVPTVIAVCAEPSGATCVGAGA | | |
| | | HPDPERALKSALVEIASDFQVVAEHGKDRA | | |
| | | AEIEWMLNDFSLVRAMEDHADLFALPQARQ | | |
| | | YIDHWLHPIRRHVTLDSLRRAPEAGVSVRD | | |
| | | DLAATLHSCRDAGFEPLAVDMRSQIANRMG | | |
| | | LACWKVVVPGLIPIDFGSYQRAVHMPRLAK | | |
| | | RVAAVTGVPASPASFVANPVPHPFP | | |
| 86 | 228 | MSQWCEPGTAAVTYADLIYHRVRDGMLIND | | |
| | | WTVDWSKEPLTHSFYPEAAAINLPLPGEGA | | |
| | | DVERGVAGNAPEFLASLLHISYGITGVRLT | | |
| | | INRNDHMLVYRNAEYAKWGRTTASGGGRYC | | |
| | | VDFYLVDSGIGHDQLDAGMYHYTPLRHAWE | | |
| | | LLTAGDFTGRVARAQGYARAADRYLIMTIN | | |
| | | YWRSGFKYNDFAYQATAMDVGTVMATMAEA | | |
| | | AGDRLASSWDMWVNEDQLAPLLGLDQTKDG | | |
| | | IYAVQAWGNERDVNAGCEPDVAAFPGRVTS | | |
| | | NYPGTIDFTTTIALQNDMMGFPQRPAELLP | | |
| | | NAVSDESPNGIAGNWWSDLMRRQSSFGRET | | |
| | | GRGFGKEDLLGLLSRADRVSALVAPPSSVV | | |
| | | WEYLVYVARVDDVEPGLYRYRPFSGDLELV | | |
| | | STEDQSEFLASTYFLKNYDGKKAAATVIPC | | |
| | | ANVYKCARRWGVRGYRLVNAVIGAACQALS | | |
| | | VEAARRGLGSGTALGFDAEAHADHAGLDKE | | |
| | | SMTPMLMIMTGVDDPLSGQFHSNATARRLV | | |
| 87 | 229 | MSVLDPYCQVRIGGLDYSHASELLDPTLAD | | ['Lantibiotic dehydratase, N |
| | | HVSQVREAYARLQAHAEDISDLLYPLIGGA | | terminus', 'lantibiotic |
| | | EASTKTGLINVRRHLYNGKYAQAWERLEEL | | biosynthesis protein'] |
| | | DETSRESVRDALGSFATDLQTFITGLDYVR | | |
| | | NHFDDALASEREKLVAHVANPLFMAGIAVS | | |
| | | SPSLVPALLRLTSHVNKGKVDKKDRKAERS | | |
| | | LQSYVLRAATKTSPFSTLGPIAIGYTRQAE | | |
| | | VEQSHRSVSLPSLYPVVRALHALAEDPKNL | | |
| | | AGFEVRVSDYVRGTDGVVSVDRTQWDFKDA | | |
| | | STRTDYAKPTESNVIINQRPLVDAVYQILG | | |
| | | GALMTFGELNDELSRQSGIPPETTLELLAD | | |
| | | LMRLGYLHVPALSIHPHDAPRIDGIVDEVR | | |
| | | QAHPELGCLIAQFVERSRSFSRLEDPQMRQ | | |
| | | REIGDIQELVASIYSLAGVDGDMPRSVLYE | | |
| | | DVVAGGFTGSELAQLELSDSEVSEIFLLLD | | |
| | | LLDDSHVKSALMEGYFDSRQRLEISATDFI | | |
| | | DGFIDELFDSFEAYDLGGIADDDLPDDPWL | | |
| | | RWGEAWRWVLARRRFVDHLSRYVATHPRGS | | |
| | | HAANLRDVGAVDISEALAQAAAILQRPQHA | | |
| | | FRHANILIQFDGEAGSVILNDAFGGIGFQI | | |
| | | SRFTHLLDEPAHEYLADVERLAKSKGVRLV | | |
| | | ELSGGALFSNLNLHEPLFSTSLVLPGEPVS | | |
| | | SRDVPAIRLEDLVVTKRDGVLVLTDGCEDI | | |
| | | HPVYAGYLVPAATPRRSQVVSLFAPSAQIS | | |
| | | RKLTSLVTTTPGIETIAVIPRLTLGRIVVA | | |
| | | RARAIMATDALPTDSPLEADGYLAWLRFWA | | |
| | | DNGLPERCFVKIIDEAVQAEKPSYFDIRSV | | |
| | | ISCSTLLNDVKNAEGKAYVEVAEVLPASPT | | |
| | | ATHDGRSVVNEYMLGISLMGGSDA | | |
| 88 | 230 | MPDTMVIPEVLADEAELRATHVWRAYHIFY | | ['Lantibiotic biosynthesis |
| | | GGNPLIVLRECILPLAEDLQREGVILDYFF | | dehydratase C-term', |
| | | INYWLEGSHVRLRLRVRPERVEELDARVLE | | 'lantibiotic biosynthesis |
| | | RVRQYLAASPSYHPMAELADNNFYESLFAG | | protein'] |
| | | EFTDADRPKYFDANGDPMFAENNSIEVREY | | |
| | | EPEWLRYGGEVGMLISERQFVESTQLMVRL | | |
| | | MHLGNLGVRTILLGIASQISFITAVCLLQD | | |
| | | ADLVEDFFVAYHHRWADGYDTNPAYGTEEG | | |
| | | RRKHQVTVENLRKKIVPRADAIRRGDVGDL | | |
| | | PQILRDWVQVCLKVRFQIEQACEHSALRFK | | |
| | | YDDGVRSVTNVDDAAWSLCHSYIHMTNNRM | | |
| | | MVSVADEAFLAYQLVEAMRGSDD | | |
| 89 | 231 | MTKSVIDWVPRLNPLLRVGPPRWNGTAIVT | | ['putative peptide zinc |
| | | DIGIDGEERLIEIGLAERCVLDEVDGSRDV | | metalloprotease protein'] |
| | | STIASELSAKGMPVDTTRIVGVLNRFAYVG | | |
| | | AVERPFSMKAGLADIDWAANEGQRVRPDQI | | |
| | | AGAADSGAGLGLWRKLTFLAHPVVFGILVV | | |
| | | AGIAGAAFLAINLSDALATVLDATVWQAIL | | |
| | | AGVAAVVWTGAVTMLHESGHGALFHHESTR | | |
| | | SPFLALTRFGLILMPNTHMPGFSLLGARER | | |
| | | ARVLAAGPLVSMVFAALPVVLFVTVDEPQV | | |
| | | RIIAAMCMCCDALIIALGISPFPNTDATRL | | |
| | | IEAWVGVDQLQAVAFRTLVGKYSLPAGLPL | | |
| | | RSKVAIRLYPVLLLDTIALWLAVIVCILN | | |
| 90 | 232 | MNLQNLAAELDSFTAETFEVKDYVDYADMA | | |
| | | FGSTCSSSSSSSSSCSSTCTSCCTSTSSCA | | |
| | | TA | | |

### HMO Utilization Genes

HMO utilization genes were detected in PB-STR-083 using a functional genomics pipeline built with CENTRIFUGE™ (Kim et al. (2016) Genome Research 26:1721-1729). The pipeline annotates a genome with functional annotations including KEGG ORTHOLOGY database (KO) numbers. Lists of KO numbers associated with HMO utilization genes and HMO utilization gene clusters were obtained from published research (Henrick et al. (2021) Cell 184: P3884-3898). The Blon gene accession IDs associated with each observed KO value in PB-STR-083 are listed in Table 20 where the genes are grouped by the HMO utilization gene clusters from Henrick et al.:

TABLE 20

| | Number of | |
|---|---|---|
| Cluster | Genes | BLON IDs |
| H1 | 20 | Blon_2331, Blon_2332, Blon_2334, Blon_2336, Blon_2342, |
| | | Blon_2343, Blon_2344, Blon_2345, Blon_2346, Blon_2347, |
| | | Blon_2348, Blon_2350, Blon_2351, Blon_2352, Blon_2354, |
| | | Blon_2355, Blon_2357, Blon_2359, Blon_2360 |
| H2 | 4 | Blon_0243, Blon_0244, Blon_0245, Blon_0248 |
| H3 | 4 | Blon_0247, Blon_0423, Blon_0425, Blon_0426 |
| H4 | 12 | Blon_0625, Blon_0641, Blon_0642, Blon_0643, Blon_0644, |
| | | Blon_0645, Blon_0646, Blon_0647, Blon_0648, Blon_0649, |
| | | Blon_0650, Blon_0651 |
| H5 | 7 | Blon_2171, Blon_2172, Blon_2173, Blon_2174, Blon_2175, |
| | | Blon_2176, Blon_2177 |
| Urease | 12 | Blon_0104, Blon_0105, Blon_0106, Blon_0107, Blon_0108, |
| | | Blon_0109, Blon_0110, Blon_0111, Blon_0112 BLIJ_0113, |
| | | Blon_0113, Blon_0114, Blon_0115 |

### Bacteriocins

Using ANTISMASH™ (Blin et al. (2023) Nucleic Acids Research 51: W46-W50) bacterial version, the PB-STR-083 genome was searched for bacteriocins, peptides known to have antimicrobial and immunological properties relevant to the infant gut environment (Benítez-chao D. et al. (2021) Frontiers in Microbiology 12), with 3 signatures observed. The observed signatures are listed in Table 29.

### Antimicrobial Resistance Genes

The ORFs found in the genome for strain PB-STR-083 were BLAST searched against the NCBI Antimicrobial Resistance Database and no antimicrobial resistance genes were observed. Antimicrobial resistance genes are increasingly common in infants (Shan Y. et al. (2019) Nature 574:117-121) and pose potential health issues (Samarra A. et al. (2023) Gut Microbes 15:2194797).

### Virulence Factors

The ORFs found in the genome for strain PB-STR-083 were BLAST searched against the VFDB (Virulence Factor Database) and no virulence genes were observed.

PB-STR-103: B. breve

Persephone strain PB-STR-103 is a member of the species B. breve. Comparative genomic analysis of PB-STR-103 was done with the published B. breve genomes in Table 28. The type-strain of B. breve is GCF_001025175.1. PB-STR-103 is differentiated from the type-strain by the following values:
accession: GCF_001025175.1,
ani: 98.4%,
coverage: 81.6%,
product: 80.3%,

The most similar published genome to PB-STR-103 is GCF_002838305.1 (determined by the strain with the highest ANIb product). PB-STR-103 is differentiated from GCF_002838305.1 by the following values:
accession: GCF_002838305.1,
ani: 98.6,
coverage: 87.6%,
product: 86.4%,

Table 21 provides a list of the unique open reading frames (ORFs) from PB-STR-103. These ORFs were determined to be unique by BLAST searches with the ORFs from the above list of published B. breve genomes. If an ORF from PB-STR-103 has no corresponding ORF in any of the published genomes (with a sequence identity greater than 60%) it is considered unique and included in the table. If an ORF had a sequence identity greater than 20% but less than 60%, the highest sequence identity to an external strain is shown in pident. Where functional annotations were possible, they are included in the table.

TABLE 21

| | SEQ | | | |
|---|---|---|---|---|
| | ID | | | |
| index | NO | AA Sequence | pident | function annotations |
| 1 | 233 | MARNANKNQVYEKLSEALNWCNAHNDNAQQ | | ['Sinl restriction |
| | | RFNNCVNNVIDANFHAERNQEMKSLFPVLL | | endonuclease'] |
| | | LNNLSGYRPGITMKETTLDLHSPDFLDLYF | | |
| | | TAWAQKYFKAWETLPSHRIAKPKEAATDPA | | |
| | | LIKMVEPQAGNIYIARDWAAYHNLFMSAEN | | |
| | | VGGNLLEEYIYTKVHDYGWTWCRGEVLTAV | | |
| | | DFCSMDKERFIQIKNKSNTENSSGKGFRED | | |
| | | HNADKWYRMEAKKKNGLVVTRWPELIQIIQ | | |
| | | EGAPDGVTVPDDLMTENSYLEFVRDAAARN | | |
| | | RQLITDKEL | | |
| 2 | 234 | MGVSPSVITRLSNTGKLDCISVGARKAFKQ | | ['Replication, recombination |
| | | STVDSYLAQHNQEHAAADHCRKSTELPKIV | | and repair', 'C-5 cytosine- |
| | | ALSFFTGAGGLDLGMEAAGIHSLLYCENNR | | specific DNA methylase', |
| | | ECRMTINHNRPDAALIGDINQYEALDILRL | | 'DNA (cytosine-5)- |
| | | ANIPESRDVDVMFGGPPCQAFSTAGARRAF | | methyltransferase 1 |
| | | DDARGNVFLRFLKLAADIKPRYLVIENVRG | | [EC:2.1.1.37]', 'DNA-cytosine |
| | | LLSTPFPTEEGGKPVRGGAMRVILNKLDDM | | methylase (Dcm) |
| | | GYAVSFNLYNAANFGAAQIRERVVIIAKRE | | (PDB:3LX6)'] |
| | | GQPAAWLTPTNSSDAAWGLPQWNTFREVTK | | |
| | | NIEQGSQHYIQFPEKRLKFFRMLKAGEYWT | | |
| | | SLPKTAQREAMGKALDLGGGKTGFYRRLAW | | |
| | | DRPSPTLVTSPIMKATDLCHPDELRPLSIE | | |
| | | EYHVIQGFPDDWWIAGELSDIYRQIGNAVP | | |
| | | VKLGEAIGQALINDMNGIPNKSDWDDFPYS | | |
| | | RYTHTSNKTWNLA | | |
| 3 | 235 | MAQRTNNGNVDNGIVIRFEKLSRQSIGNIT | | |
| | | KIVGMVRAKYLIPIIDRLDLQANPRNSKTG | | |
| | | TVTKEIQRSIENDPLLFPFKTKGLLLASSH | | |
| | | YEEMERGRIRVTFDDRTTEGILDGGHNTLA | | |
| | | IGLLILDRSMGYARAAGLADVKMPAGAKTW | | |
| | | ADFKQLWKKNRDLIQKYQESVRKSENSQPE | | |
| | | LGTQDEDLSFYVPVELLVPTDPDNPICAEE | | |
| | | FSNNLLEICVARNNNAELTTGAKANQHGYF | | |
| | | DDLADALNKVDPNVAERIEWKTNDGGDIKV | | |
| | | QDFIALTWLVLKWLCPVTDSEGKKVEAPSM | | |
| | | RKLYSGKGSCLALFERFMSSEDITSQDPSS | | |
| | | YRHGLRNPDVYRAFELAAQIPALYDRIYKD | | |
| | | FPDLYNQAGGSYGRITAVKSLNARTTNKTT | | |
| | | PFSNMPIDTVSPDGYITPLVCGLSTLVDAK | | |
| | | TMKWKTDPNKFLDRWLPSIVKRYMQVFVPC | | |
| | | DYDPQKVGKSAASYSAVEDAYKMAFANIL | | |
| 4 | 236 | MTEENKTPQAATEEQLLSATAVDGQTITLT | | |
| | | PISLEEAFKVFDGKEDNSE | | |
| 5 | 237 | MVGDNVRLNDVPLERDESLDWHKMFGSLTV | | |
| | | SIYHRFGSIYEVSVIGTVSGGSHYNYTTIT | | |
| | | DQLPHKFESRSVPGFMHSSYDSAGVVFEVI | | |
| | | DGRVLQLLGKSVSQDMSYDVRGMFVRVYA | | |
| 6 | 238 | MANDNALTWGELTEKKTMDDQGVAVDIRGV | 41.5 | ['Inorganic ion transport and |
| | | NKRFVTQQGDHVTALHDINLTINKHDFICV | | metabolism', 'ABC |
| | | VGRSGCGKTTLLNMLAGFEKPSDGELISGG | | transporter', 'NitT\VTauT |
| | | VAINGPSPKRGVVFQKPPLYPWLTVRKNVE | | family transport system ATP- |
| | | FGMKMQGVPATERKEKADHFLDLVGLTSAA | | binding protein', 'ABC-type |
| | | DRRPYELSGGMQQRAQIARVLATEPDLILM | | nitrate\Vsulfonate\Vbicarbonate |
| | | DEPYGALDALTREKLQNELLRIWRERHSTV | | transport system, ATPase |
| | | FFITHSVDEAIFLATRVIVMSAHPGTVKMD | | component (TauB)'] |
| | | IPITLPRDPDDPDNMEKVRAMPEFVKLREE | | |
| | | ITQAIYVQDDQE | | |
| 7 | 239 | MKILRETTRETVRKIGALLVSATVLTSLAG | | ['Inorganic ion transport and |
| | | CGKMPTADELVGNTSGAAVADCPVPSAKNE | | metabolism', 'taurine transport |
| | | DEKGTIRIAWQAIPNADLIVKDKGLLEACL | | system substrate-binding |
| | | PNAIIQWSQFNSGGDVIQAFGSNSLDIGLA | | protein', 'ABC-type taurine |
| | | GSSPAVRAASAPLNLPVKVIWIHDIIGDAE | | transport system, periplasmic |
| | | SLVARGGDYKSIKDLAGKNIAVPFGSTSHF | | component (TauA) |
| | | SLLSALHNAGMNETSVNLINLDPDKMSAAW | | (PDB:6SSY)'] |
| | | TRGEIDAAWVWDPVLSKLKADGGTIVTSSA | | |
| | | ATAKTGSATYDMELATDAFIKANPKVMETW | | |
| | | TAVENYAAGLISSKEADSAESISTILGNSV | | |
| | | ADVKKQFKGYTYPQAKDQSDIFHGQLPGIF | | |
| | | KDTAEFLKTQGSLDKVSDDYSSVLYTDAID | | |
| | | QVAKQ | | |
| 8 | 240 | MSEATTPTSNASNASDTPVVAGTAVAGTSI | | ['Inorganic ion transport and |
| | | VEGAATTQRKPMPAWMKSKKIQPIISLVLL | | metabolism', 'Binding-protein- |
| | | IAVWWGVTSAGLINSLYLPGPKAVWDAFIE | | dependent transport system |
| | | ANSCRPASAGSSRIVCGEQQYFLWQHLVVS | | inner membrane component', |
| | | LERIGIGMALGIVVGVLVGFILAEIGWLNR | | 'NitT\VTauT family transport |
| | | IILPYINFIRALPPLGYIGLLIVWFGIGDT | | system permease protein', |
| | | TKIWLLFLAAFPPIVLATVDGINGVNRDRV | | 'ABC-type |
| | | NSALSLGASRPKAFLFVVFPSALPSIMNGI | | nitrate\Vsulfonate\Vbicarbonate |
| | | RLAVGFAWTTVVAAELNNGIPGIGALAYLS | | transport system, permease |
| | | GTELNTPLTIACIFVIGIAALLLDWLILGI | | component (TauC)'] |
| | | THLVTPWVGKE | | |
| 9 | 241 | MGKTHSTKSASVDGSSSVAVRPFGWRDKIG | | ['Carbohydrate transport and |
| | | YMCGDFGTDFMFVFAGTWFMIFYTKVMGIP | | metabolism', 'MFS\Vsugar |
| | | GTIVGTLFLLARVLDAVMDVTVGVVVDRSK | | transport protein', |
| | | DHPGGKFRVTMMRFTAPLVILSFLMYQTFA | | 'glycoside\Vpentoside\ |
| | | IDAAMWVKIVYMSVTYVVWCFFYSCVNIPF | | Vhexuronide: cation symporter, |
| | | GSLASTISAEADDRTSLSTMRSMGGTIGGL | | GPH family', 'Na+\melibiose |
| | | ILGIVAPIVIYQKVDGHQVIRGGDGTHIFV | | symporter or related |
| | | IVAAVVSIAAFIGYLICYFNVTERVHPDPD | | transporter (MelB) |
| | | QNAKEGEKRSPIHMIANAFSSRSMLGIVAA | | (PDB:4M64)'] |
| | | ALCLLVAQLFSNQLMTYVYTDVFGSAALAS | | |
| | | ISGLLGTAVMFLVVPFVKPLTRRFGRKSIC | | |
| | | SFASALGAISMLLLFVIQTRSGIVFLVGAV | | |
| | | FMYFALMGENLVIWAMITDVIDDIEVTRDT | | |
| | | REEATCFSCYSFARKIGQAVAGELAGLSLD | | |
| | | WVGYQSGATAVQSSATKTALYNVATLVPGV | | |
| | | FFLLTFVCLVIIYPLSRDRVLYNVEVLRVK | | |
| | | RKGITGEEARLLVKYEGITPTGTTPEGSTI | | |
| | | EWPDFHELERARELSARTGIAIRDPQAEAS | | |
| | | TRIEQ | | |
| 10 | 242 | MATIYTGGTIRTMTAPDATAEALLTDDRGR | | ['General function prediction |
| | | IAFVGSLDEARNRAASMITVGGRVPEEHDL | | only', 'Amidohydrolase family', |
| | | AGACLMPGFIDSHSHFAGMGQRLTDADLAG | | 'Predicted amidohydrolase |
| | | CADFAEIGTRLKTFLAAHPLPAGGVLHGEN | | YtcJ (YtcJ) (PDB:3ICJ)'] |
| | | YDQNELAEGVHPDRYVLDAMLGDVPTVISH | | |
| | | VSGHVIICNTALLQLIGLDLDADAPAGGVY | | |
| | | GKGADGKLNGYFAETPAIMPILMHPAVMPS | | |
| | | QSVEDLADAIQNDYASHGITACQDGATAPG | | |
| | | YAERFVKLAENHQLKLDLTCYPMMGQDLDA | | |
| | | VFESVGSYASGGTSEGIQYLNHLRFGGVKL | | |
| | | FVDGSPQARGAWLSEPYEPLPASAKVHEPA | | |
| | | GYRGAGILSDEAMRAFLDHALERGWKVMAH | | |
| | | CNGDAASEQFLTQYTAAYRASSKPDKFDLR | | |
| | | PVMVHCQLTRNDQYARMAAVHMIPSIFVSH | | |
| | | CWFWGDAHIKNVGFARASRISAVHDALDYH | | |
| | | LPFTFHTDSPIVPPNLIFAAWCAMTRVTKH | | |
| | | GVELDQSQCVGAWEAFSAITRNAAYQYGEE | | |
| | | SRKGTLEAGKLADLTILDADPLAASADKAE | | |
| | | AAAHVRELKVLETIKEGTTVWTA | | |
| 11 | 243 | MLWDYQQPVAIRFGNGRVREIKDVAAEMGL | 27.5 | ['Energy production and |
| | | TEGGLLVSEHLFAKNGTAEKIVKDSEGAIS | | conversion', 'Iron-containing |
| | | EIFSDFSPNPDVTEVDKAAALIRDKHLKFV | | alcohol dehydrogenase', |
| | | VAMGGGSAMDLAKSAASIALTNDSIAEYHG | | 'alcohol dehydrogenase |
| | | TGKAMPQEHLPIIAVPTTAGTGSEVTCVSV | | [EC:1.1.1.-]', 'Alcohol |
| | | LTNRALGKKAPIVSDGFFPSVAIIDPELTY | | dehydrogenase, class IV |
| | | SVPPHVTASTGMDVLSQAIEGYWSKGHQPI | | (EutG) (PDB:6AHC)'] |
| | | CDACAIHAAPLVFKYLPIAVAEPDNAEARQ | | |
| | | KMCEASVIAGLAFTLPKTTSSHACSFPLTN | | |
| | | IHGIPHGEACGLTLDWFARINKDAQHGRVQ | | |
| | | EFARAIGFENVDAMADAIHELKVKVGLRTG | | |
| | | LKDLNLNAEQIADLVRISRHPNLYNNPVEI | | |
| | | TDEMLQDMYEHLAATD | | |
| 12 | 244 | MSESLATKELTGEEVAELHRKYVMQSWHKQ | | ['Coenzyme transport and |
| | | GEPVTPVKSAKGIYVYDYDGNKYADMSSLL | | metabolism', |
| | | VCSNLGHELPEIVDAIKDQASKMCFMAPAY | | 'Aminotransferase class-III', |
| | | ASEPKSKLAKMIVDLAGNDFYQRVFFTNGG | | 'taurine---2-oxoglutarate |
| | | ADSNENAIKMARMVTGRQKIFSCYRSYHGS | | transaminase [EC:2.6.1.55]', |
| | | TLGASFASGDWRRFATEAGGAAPSFVHEMN | | 'Biotin biosynthesis', |
| | | PNMYEDGFNRGEDDDKVTALYLKRLEDQLI | | Adenosylmethionine-8- |
| | | YENPDDVAAILMESIVGANGVILPPKGYME | | amino-7-oxononanoate |
| | | GVRALCDKYGILMICDEVMAGWCRTGKMFA | | aminotransferase (BioA) |
| | | WQNFDIKPDIFTFAKGVTCGYVPLGGVVVS | | (PDB:1MLY)'] |
| | | KQISDYFTDHVLQCGLTYSGHTLACAAGVA | | |
| | | AVNYYVEHDIAGHVKEMEGILKPFLEEMEA | | |
| | | KHKCVGESRCIGLFSAMTIVKNKETRELMS | | |
| | | PYHTANPVMGKIMGALKDKGFLTFGRETNV | | |
| | | NICPPLTITAEELKAELPKVDEVLTWVDDN | | |
| | | FCD | | |
| 13 | 245 | MTVRLKELVEQAADKNMVLAAGKNGLDKPV | | ['Transcription', 'Purine |
| | | RWVHMVENEEIAGFLEGQEIAFTTGIGLET | | catabolismoregulatory protein- |
| | | QEDLYPLVKSAYASGATGVVVNIGPFIHQI | | like family', 'PucR family |
| | | SPETIRFCDEHDFPLFKVPWSVHMAQIMHS | | transcriptional regulator, |
| | | FSLAITMSEKHSMELAAALENAIFHPDREE | | proline-responsive |
| | | MYLEYLEQSGFGKDWNYCVAVESACADAEH | | transcriptional activator', |
| | | GGHDAELIAKYSREAESLITANQWRVALVR | | 'DNA-binding transcriptional |
| | | IEDRLVLVFARYTAEQVELMVREIIAAIRR | | regulator, PucR\VPutR family |
| | | RGVILERTYIGVGKVTKSARCIGKSYNQAL | | (PucR) |
| | | KLERLQHLRGRVGEVALYDNSGIDKLLLAV | | (PUBMED: 14990804)'] |
| | | SDRAILEDYYQDSIGPLVEYDRVNGTDLTE | | |
| | | TLHAYFQFSGSVKETAVSMFVHRNTVSYKL | | |
| | | NKIEDILGVSLSDFRTREFLSVGLQVREIL | | |
| | | DC | | |
| 14 | 246 | MGESVRDMLFVVVLFAANVIQAITGFAGTV | | ['Inorganic ion transport and |
| | | LAMPFSMLLLGTDTAKVVLNITTLLVCLWL | | metabolism', 'Sulfite exporter |
| | | GVQHRAHIRWRILGEMVGLMAIGMVAGVAL | | TauE\SafE', 'uncharacterized |
| | | YAALPLAPLQKAYGVFIIVIALKNLIWPAH | | protein', 'Sulfite exporter |
| | | AEPPHWLLIIIVLLAGVIHGMFISGGALLV | | TauE\VSafE\VYfcA and |
| | | IYAAVRLKDKNEFRATMACVWVALNSVLTV | | related permeases, UPF0721 |
| | | QQGVAGVITPHALALTAVSIPPLIVAIIIG | | family (TauE) |
| | | NKLQKRVSQQAFLKLTYVLLVISGASIVL | | (PUBMED:21183667;22797525)'] |
| 15 | 247 | MTCATFPGMRHGLGLLPGLAVRSIDPACYF | | |
| | | TLTVRNGVNRLIHIRKIDKRHCTVLRRAGG | | |
| | | DMLVEA | | |
| 16 | 248 | MLPRSHPAGNLPQAASGIESLAVDACGRPF | 52.1 | |
| | | PARWPCQTSVRRHHFCQPMSCGRLPAKPDG | | |
| | | P | | |
| 17 | 249 | MTAPKVAASPQDIAILNLNMLMQLEGRYRK | 52.8 | |
| | | DLAEYIGRRPQNLSRMMSGESNWALNDMWK | | |
| | | AAEFVGVSLDVLTDPTLTPAKALSIIGERR | | |
| | | NDNDGNGGLPVVNVDDERLRGGAWKAQAMV | | |
| | | LAA | | |
| 18 | 250 | MGASVDVATWVSVGCAAVSAVFAGVTVWWP | 41.7 | |
| | | WHTRPAPDLRHEKDEFSVTRESMAHLLVTC | | |
| | | GLQRPRLLVRWRNDGDGTAYAVTIKAADGS | | |
| | | CAVRMAVPDTSKPSGFDFVDSVGKMEPGES | | |
| | | FEAIILPTSTEDVGKPVVLLDWKESPTRLR | | |
| | | MGHRSERVALPYRLPGKRPLLRQERILALH | | |
| | | MIRTTAAEYGYPYEQFASQMLGIDLEDLDP | | |
| | | WSAPDSKGKTESPDSGE | | |
| 19 | 251 | MPEPPPKPARRGAKTVGHQGGADLVVVVTA | | |
| | | GHLGHGLDVADILGHKHEHDGQEHRQDGEV | | |
| | | GLRQVELREADPCGVINGGEVDLAAEAGIN | | |
| | | VADDHADQNIESTKQSLEQHGDKQHGQQSH | | |
| | | DCGIRRGLEIGPYGGRQIEADNGHDRTVDH | | |
| | | RRHDDVDPLGTGVMHEHTDQSQQDTGAHDA | | |
| | | EAGDRDALVGGGDCGDRGDEAEGRAQIAWQ | | |
| | | HVLVDEQEQCGGHGGKEQGGGRIKARENRH | | |
| | | QEGGAEHGDDVLRADAGGTRPSQTFIRLDH | | |
| | | LAGLQRLAVAMQLPLEDIGHYYSPRSRRQA | | |
| | | RHTHTSDRAVRHCIS | | |
| 20 | 252 | MDRATDEHSQQERKPATSCLARLSGATVRR | | |
| | | MPSADRFAYAAATVLMEYPMAIMQDMASSA | | |

### HMO Utilization Genes

HMO utilization genes were detected in PB-STR-103 using a functional genomics pipeline built with CENTRIFUGE™ (Kim et al. (2016) Genome Research 26:1721-1729). The pipeline annotates a genome with functional annotations including KEGG ORTHOLOGY database (KO) numbers. Lists of KO numbers associated with HMO utilization genes and HMO utilization gene clusters were obtained from published research (Henrick et al. (2021) Cell 184: P3884-3898). The Blon gene accession IDs associated with each observed KO value in PB-STR-103 are listed in Table 22 where the genes are grouped by the HMO utilization gene clusters from Henrick et al.:

TABLE 22

| | Number of | |
|---|---|---|
| Cluster | Genes | BLON IDs |
| H1 | 6 | Blon_2331, Blon_2332, Blon_2334, Blon_2348, Blon_2357, |
| | | Blon_2360 |
| H3 | 1 | Blon_0423 |
| H4 | 10 | Blon_0625, Blon_0641, Blon_0643, Blon_0644, Blon_0646, |
| | | Blon_0647, Blon_0648, Blon_0649, Blon_0650, Blon_0651 |
| H5 | 7 | Blon_2171, Blon_2172, Blon_2173, Blon_2174, Blon_2175, |
| | | Blon_2176, Blon_2177 |

### Bacteriocins

Using ANTISMASH™ (Blin et al. (2023) Nucleic Acids Research 51: W46-W50) bacterial version, the PB-STR-103 genome was searched for bacteriocins, peptides known to have antimicrobial and immunological properties relevant to the infant gut environment (Benítez-chao D. et al. (2021) Frontiers in Microbiology 12), but no bacteriocin signatures were observed.

### Antimicrobial Resistance Genes

The ORFs found in the genome for strain PB-STR-103 were BLAST searched against the NCBI Antimicrobial Resistance Database. Antimicrobial resistance genes are increasingly common in infants (Shan Y. et al. (2019) Nature 574:117-121) and pose potential health issues (Samarra A. et al. (2023) Gut Microbes 15:2194797). There were 3 antimicrobial gene signatures observed. Table 23 describes each of these signatures:

TABLE 23

| class | sseqid | pident | product_name |
|---|---|---|---|
| AMINOGLYCOSIDE | WP_001255866.1 | 100.0 | aminoglycoside |
| | | | nucleotidyltransferase ANT(6)-Ia |
| STREPTOTHRICIN | WP_000627290.1 | 99.4 | streptothricin N-acetyltransferase |
| | | | Sat4 |
| AMINOGLYCOSIDE | WP_001096887.1 | 100.0 | aminoglycoside O- |
| | | | phosphotransferase APH(3′)-IIIa |

### Virulence Factors

The ORFs found in the genome for strain PB-STR-103 were BLAST searched against the VFDB (Virulence Factor Database) and no virulence genes were observed.

PB-STR-119: B. breve

Persephone strain PB-STR-119 is a member of the species B. breve. Comparative genomic analysis of PB-STR-119 was done with the published B. breve genomes listed in Table 28. The type-strain of B. breve is GCF_001025175.1. PB-STR-119 is differentiated from the type-strain by the following values:
accession: GCF_001025175.1,
ani: 98.6%,
coverage: 83.3%,
product: 82.2%,

The most similar published genome to PB-STR-119 is GCF_002838565.1 (determined by the strain with the highest ANIb product). PB-STR-119 is differentiated from GCF_002838565.1 by the following values:
accession: GCF_002838565.1,
ani: 100.0,
coverage: 98.1%,
product: 98.0%,

Table 24 provides a list of the unique open reading frames (ORFs) from PB-STR-119. These ORFs were determined to be unique by BLAST searches with the ORFs from the above list of published B. breve genomes. If an ORF from PB-STR-119 has no corresponding ORF in any of the published genomes (with a sequence identity greater than 60%) it is considered unique and included in the table. If an ORF had a sequence identity greater than 20% but less than 60%, the highest sequence identity to an external strain is shown in pident. Where functional annotations were possible, they are included in the table.

TABLE 24

| | SEQ | | | |
|---|---|---|---|---|
| | ID | | | |
| index | NO | AA Sequence | pident | function annotations |
| 1 | 253 | MIGFSPSLSVEHASASMPLANAESGLVPIV | | |
| | | HATGMPSWQSITGDGQALPAGIENSVRSVT | | |
| | | HGMFGRSAWKSPSTRFAGALVSSPLYEPCR | | |
| | | FALLDRGHQAVPGHEPHDLLRAGHDSHAPQ | | |
| | | LQVDPLVPVPALAVLERLAHELQ | | |
| 2 | 254 | MRRSPATTTCPETERPARASQTAGKVATYN | 50.0 | |
| | | WTDKDGQGGCYGGNLYNEIKGIWERADRNE | | |
| | | KALAQLTALVAAQQTALDTLAKSLGANPAD | | |
| | | IAEIVAQAVTAKLDSIDVTFTATSK | | |
| 3 | 255 | MVQIKEEIVNQGHGYLSPSLFAVHSTANPG | | ['Cell |
| | | ATARNHRDLWSRGYDYAVHLTSDWKEAIHC | | wall\Vmembrane\Venvelope |
| | | VPYDRLCWQVGNGNTTCEGIEICEATNASD | | biogenesis'] |
| | | FWKGIDIAADVIAQRLRAHGWGVDRMHPHS | | |
| | | WFSQTYGGSDHTDPIPYFSRYGYTWPAFIQ | | |
| | | LVQQKLSGNTGTQEVDIMAAMMIRNDSTGV | | |
| | | IWYCEPGKGRTALTHPDQANLLQQAGVPLI | | |
| | | HGNNGAPWWSRFDQIDSMVRGTMKKLGV | | |
| 4 | 256 | MNLPEGLPAWAYIVVSALVLAAQIVTAIWI | 50.9 | |
| | | NHRGDERDRATRGEITNNHEMPLRDDLDDK | | |
| | | ALRTLDAIESLRGAVDGLRDDMNGEFATVN | | |
| | | RRITTTEQNLIELRHEVNDLRRGGNNHQ | | |
| 5 | 257 | MKYSAKRNAHGSTPSYVDRDSLTPPLHLYT | | |
| | | GSGVYKDAGSGGYATMWTFGQFKTQFGRDW | | |
| | | NNDVVVAAINGDWDANGRQVTSVRVTPSGN | | |
| | | RIDVMFDGKNTAPIRVNWAVMWRG | | |
| 6 | 258 | MAYVKKTNLRGPQGPAGPQASTEQIFDKAW | | |
| | | PIGTVLETNSDSTPPTYNTGWKKLPNIMGR | | |
| | | GFLWQRIG | | |
| 7 | 259 | MAYVKKLNLTGPQGPQGPKGATGPQGPRGP | | ['Collagen triple helix repeat |
| | | QGPQGAKGDAFAIAKTYKSVAEMNAGYATD | | (20 copies)'] |
| | | GVLNGQFVMIDTGNVNDADNAKLYVKGAKG | | |
| | | YTYITDLSGATGMTGPQGPQGPKGETGAQG | | |
| | | EKGATGAKGDTGPQGPQGVKGATGAAGAKG | | |
| | | ETGPAGPAGPTGATGPAGPQGEKGATGAKG | | |
| | | ADGNTITYGTSAPAAGATGKAGDMYIQSNG | | |
| | | DLYIFE | | |
| 8 | 260 | MSIQLIDKIKQKNDGNFYLVDAVDVEYNKK | | |
| | | SLIDALKAGDIIPAGGGTSAGTFHVANINI | | |
| | | GSNTAFDSSKLTPSTVSAGDLIVDANGAFY | | |
| | | TVAKVDGTTVTPSAALTADGGGTLGFKGAK | | |
| | | GDKGEPGAQGPAGPQGPKGDAFTIAKTYAS | | |
| | | VAEMNADYRNADIPIGSFVVISTTDVNDAD | | |
| | | NAKMFVKNDTQYAFVTDLSGAQGIQGPAGP | | |
| | | QGIQGPKGESGEQGEKGANGTPGAKGETGQ | | |
| | | RGNRITVGDGDPGEPPADALAGDVYINQAN | | |
| | | GDFYQVQDQ | | |
| 9 | 261 | MANAWKKMGSLRGPAGAGADVATSEKAGVV | 55.4 | |
| | | KPSGDFDITADGTLSLYTPMSVMSFTGGSD | | |
| | | HEIGETVDTVNLAWKLNKTPATLTLDGQEI | | |
| | | VKGEDGQFPTSQPLTKQALKANKTYTLAVT | | |
| | | DARGSKASKTTSVLFHYKRYWGVGGNPADG | | |
| | | VDSTFLLALAGSELGDSKAKTFTVNAAAGQ | | |
| | | YIWYAIPHSFGTPTFKVGGFEGGFNLVKTF | | |
| | | DHTNASGATVSYDVWQSTNAGLGNTTVNAA | | |
| 10 | 262 | MIRTLNGCNCDDLVTIIDDAIVMEGQKGES | | |
| | | AYETAVRLGYQDTEAEWVDSLHGKGITLGH | | |
| | | GDPTSQLTAREGDGYLNADNGDLCEYTSEP | | |
| | | DGSEETNDKESNHG | | |
| 11 | 263 | MTSQAIRDKVLAWHGRGYGATDTARQLGLP | | |
| | | LEEVRAIIREGDGRPKPPCKVEFIEPPLFE | | |
| | | E | | |
| 12 | 264 | MADETEPAMFDALEKALMPLNSARQLAELS | | |
| | | GIGESTLAEWRGTHTGPAYVKSGRRVLYPK | | |
| | | EAVLGFMRANLRECKEASA | | |
| 13 | 265 | MAQWNIRENDELIGPFDDAETQAISQKLTT | | |
| | | \|STRTQGGVVFSGKLADSGNDVTAYWTPGCP | | |
| | | ISFEQI | | |
| 14 | 266 | MFQMPRKATTLFCMVLGQIDLGQILVTALV | | |
| | | SAVVSFAVTVILRVWDRRTVEWLVAGEAHP | | |
| | | TYFAGEKKDMVLNLELWNTGDADAYDVRLI | | |
| | | RCNGVNPDTGKERECWETFEAGCIKAGEHV | | |
| | | VFTMKPSFASWETCWVKVVFRPSPVYRHNP | | |
| | | RCSRKYLLSKEIGNQFEYRPEESEMAVYGR | | |
| | | GKIPARPKDA | | |
| 15 | 267 | MGEARFQHVRKTPDIGEMHNLYSRDYDNQD | | |
| | | KLQNVDEILATHFAPPSASAGIAMLGDMRS | | |
| | | LAATAKCLAICSISSKVKGDLFSKRRLSVD | | |
| | | KGIFVAPASASMLSRREPSSARIFDTASLL | | |
| | | RLK | | |
| 16 | 268 | MNDHPKLTKEQQDTCERLLDKSQEAFILAI | | ['EC042_2821-Ike REase'] |
| | | ELFNRPTIRYRVEGCAFFLCNAWELMLKAY | | |
| | | IAKRDGYEAIFYPGKENRTLALEDCLKKVM | | |
| | | TNDKDPVRLNVESINELRNTGTHFVVEEYE | | |
| | | ITYGPIFQANIRNYDDRLRTYHGIEICNRI | | |
| | | PDNYLVLSVNRTDIDGESIRAKYTPEVAER | | |
| | | LLSMQNDIDLRSAEESNTKYAAYFRTEFVL | | |
| | | SKKEGIPIRVDNSAGSTARVIKQVVQPDDR | | |
| | | YPYRMSDLLKLVNRQLLRQAVKFTSGENAD | | |
| | | ARFNNYHFNLFVKCYGMKHDERYAFDRATA | | |
| | | AEKKAGRHQYTYSNAVVNFIVEEIAKDPEH | | |
| | | IVEKLRRKVEGEER | | |

### HMO Utilization Genes

HMO utilization genes were detected in PB-STR-119 using a functional genomics pipeline built with CENTRIFUGE™ (Kim et al. (2016) Genome Research 26:1721-1729). The pipeline annotates a genome with functional annotations including KEGG ORTHOLOGY database (KO) numbers. Lists of KO numbers associated with HMO utilization genes and HMO utilization gene clusters were obtained from published research (Henrick et al. (2021) Cell 184: P3884-3898). The Blon gene accession IDs associated with each observed KO value in PB-STR-119 are listed in 25 where the genes are grouped by the HMO utilization gene clusters from Henrick et al.:

TABLE 25

| | Number of | |
|---|---|---|
| Cluster | Genes | BLON IDs |
| H1 | 6 | Blon_2331, Blon_2332, Blon_2334, Blon_2348, Blon_2357, |
| | | Blon_2360 |
| H3 | 1 | Blon_0423 |
| H4 | 10 | Blon_0625, Blon_0641, Blon_0643, Blon_0644, Blon_0646, |
| | | Blon_0647, Blon_0648, Blon_0649, Blon_0650, Blon_0651 |
| H5 | 7 | Blon_2171, Blon_2172, Blon_2173, Blon_2174, Blon_2175, |
| | | Blon_2176, Blon_2177 |

### Bacteriocins

Using ANTISMASH™ (Blin et al. (2023) Nucleic Acids Research 51: W46-W50) bacterial version, the PB-STR-119 genome was searched for bacteriocins, peptides known to have antimicrobial and immunological properties relevant to the infant gut environment (Benítez-chao D. et al. (2021) Frontiers in Microbiology 12), but no bacteriocin signatures were observed.

### Antimicrobial Resistance Genes

The ORFs found in the genome for strain PB-STR-119 were BLAST searched against the NCBI Antimicrobial Resistance Database and no antimicrobial resistance genes were observed. Antimicrobial resistance genes are increasingly common in infants (Shan Y. et al. (2019) Nature 574:117-121) and pose potential health issues (Samarra A. et al. (2023) Gut Microbes 15:2194797).

### Virulence Factors

The ORFs found in the genome for strain PB-STR-119 were BLAST searched against the VFDB (Virulence Factor Database) and no virulence genes were observed.

PB-STR-321: B. bifidum

Persephone strain PB-STR-321 is a member of the species B. bifidum. Comparative genomic analysis of PB-STR-321 was done with the published B. bifidum genomes listed in Table 28. The type-strain of B. bifidum is GCF_001025135.1. PB-STR-321 is differentiated from the type-strain by the following values:
accession: GCF_001025135.1,
ani: 99.1%,
coverage: 94.1%,
product: 93.3%,

GCF_001025135.1 is also the most similar strain (determined by the strain with the highest ANIb product) to PB-STR-321.

Table 26 provides a list of the unique open reading frames (ORFs) from PB-STR-321. These ORFs were determined to be unique by BLAST searches with the ORFs from the above list of published B. bifidum genomes. If an ORF from PB-STR-321 has no corresponding ORF in any of the published genomes (with a sequence identity greater than 60%) it is considered unique and included in the table. If an ORF had a sequence identity greater than 20% but less than 60%, the highest sequence identity to an external strain is shown in pident. Where functional annotations were possible, they are included in the table.

TABLE 26

| | SEQ | | | |
|---|---|---|---|---|
| | ID | | | |
| index | NO | AA Sequence | pident | function annotations |
| 1 | 269 | MGMMPTMMRRWASWTMGWALRMSPVSHGLQ | | |
| | | ESGKGRAHTARMWEAAWLKRIRMGCAVVVP | | |
| | | GAAVMLLSGPTRL | | |
| 2 | 270 | MPPTTVADTAPSTAPGNRASAPSTVPAVKI | | |
| | | AIAMMNRVFVANLRTMNGELGIDTDSNSR | | |
| 3 | 271 | MQFYSRPGKILHDTTKKLPNHQQRSSIAVS | | |
| | | FLATPSGPAASQHVVVLLLFILGGIPICQE | | |
| | | SRVAKVVPF | | |
| 4 | 272 | MNIIISGLKINTAKGELDASPLAHKSGILN | | |
| | | LTLAENSANELIAGAFHAGLEKNDVNGAPD | | |
| | | ETFALNIDSQGDGADAGWGSLTAKSLYGTI | | |
| | | GKSSYSAGIGGAAVYRRH | | |
| 5 | 273 | MEDRISMATKRQITLRFKDEYAKASKKDKG | | ['Mobilome: prophages, |
| | | VILDRMCETLKIGRSTARRRLKEAGRAGEG | | transposons', 'Transposase |
| | | REAPRERPKRYSDRSRLLLEQVWLLMDLPC | | and inactivated derivatives, |
| | | AKYMKEMLPQWIPTLLEAGELRGEDTATVD | | IS30 family (Tra8)'] |
| | | ELLAMSAATMDRYLKPVRDAANPKGMASTR | | |
| | | PAGELLRNSITIRKAGDELDGLPGNVEADT | | |
| | | VAHCGPSLKGEFCRTLTVVDFATGWTENAS | | |
| | | ARNNAYRNLSKAEAVIEERLPFAIRSYDND | | |
| | | NGSEFINLDFISHLQAIDVEQTRSRPYRKN | | |
| | | DQATVESRNNHIVRRHAFHYRYEPAELDLL | | |
| | | NELWELVRIKANLFTPSKKPVGRASTRDGR | | |
| | | PKRVYDQPMTPWERLKRFDDEDRARGGPGF | | |
| | | IKPGRRERIERLIAETNPAELVRRIHAIQD | | |
| | | ELEQAAMPRTKRLERKLGPDMAYLNKTLAR | | |
| | | ITGADVSETDDEPAGAS | | |
| 6 | 273 | MEDRISMATKRQITLRFKDEYAKASKKDKG | | ['Mobilome: prophages, |
| | | VILDRMCETLKIGRSTARRRLKEAGRAGEG | | transposons', 'Transposase |
| | | REAPRERPKRYSDRSRLLLEQVWLLMDLPC | | and inactivated derivatives, |
| | | AKYMKEMLPQWIPTLLEAGELRGEDTATVD | | IS30 family (Tra8)'] |
| | | ELLAMSAATMDRYLKPVRDAANPKGMASTR | | |
| | | PAGELLRNSITIRKAGDELDGLPGNVEADT | | |
| | | VAHCGPSLKGEFCRTLTVVDFATGWTENAS | | |
| | | ARNNAYRNLSKAEAVIEERLPFAIRSYDND | | |
| | | NGSEFINLDFISHLQAIDVEQTRSRPYRKN | | |
| | | DQATVESRNNHIVRRHAFHYRYEPAELDLL | | |
| | | NELWELVRIKANLFTPSKKPVGRASTRDGR | | |
| | | PKRVYDQPMTPWERLKREDDEDRARGGPGF | | |
| | | IKPGRRERIERLIAETNPAELVRRIHAIQD | | |
| | | ELEQAAMPRTKRLERKLGPDMAYLNKTLAR | | |
| | | ITGADVSETDDEPAGAS | | |
| 7 | 274 | MNRKTNMPRYTLTLIDSTNQTPPIVLTAES | 48.7 | ['Replication, recombination |
| | | TRTIIRQLTKRLDPTPERRRVQGSGGFTME | | and repair', 'Site-specific |
| | | RGRVKAFRELPPDPATGNRRRATAFGATRA | | recombinase XerD (XerD) |
| | | EAERRLNEKIAELQRVGRLGYTKPPTVAEW | | (PDB:1A0P)', 'Phage |
| | | CDHWLNDICKPHLKPRTWGTYASVIANNIV | | integrase family', 'integrase'] |
| | | PSIGAVRLDELKPAHFRRMERYVMDEQGKS | | |
| | | SGTAGSAWRTLHKALEDAVLEGVIERNPAV | | |
| | | KGTAPRVALKERAALTPEQAADLIAAETDD | | |
| | | TWRLMWMLAFMTGMRQGERLGLTEDELRRD | | |
| | | GDRLIILVEWQAQHLTKREVEGLPKGVECT | | |
| | | PLGNGMYRTRPKTEKGRRAIPLPEALADQL | | |
| | | LAYIERHGVNGDGLVFHDDRGLPLTGCVER | | |
| | | RRWYRALDRVGLGHEYVPHSARHTTATILN | | |
| | | RLGLDDVTRTAIMGHSRVSTTNEIYTHVEL | | |
| | | DRLVAATDGVERAIEG | | |
| 8 | 275 | MPGNAGPVWSLPLLTVPGRMRSGMEVPELM | | |
| | | TATQVARILGVSTETLRKWRARRMCLPYVR | | |
| | | VGRHIRYRAADVAAFIERGTVMPA | | |
| 9 | 276 | MPYIDIHNERVWIPERPLRSMDEARPMRDG | | |
| | | DLWRCPACGRELDEEENDDGTITLVCPAHQ | | |
| | | VGWDAGHEDEPYPMGGLVFRPTMGGGVFIA | | |
| | | GPCMAVCLDRTSAVNAFFALGETYGLTRIE | | |
| | | GVRP | | |
| 10 | 277 | MNAFKGRGGHPHCPACWARMFRSAPLDGGP | | |
| | | ATLVCLRCGLERPLEPVAGPPEPYTRALRA | | |
| | | LDMLADALDEDVMP | | |
| 11 | 278 | MNRLRGFRLRAGMTQAELARAIGTTQSHVS | 40.4 | ['Transcription'] |
| | | EYESGARRIETMPAGEFVRLKAVPDVTDAL | | |
| | | LLDADLSGIDPDAVAAAENRVARLRRRLRR | | |
| | | RRDRASFDLLVEAELDLARRMDEREEAVAR | | |
| | | AMGSRA | | |
| 12 | 279 | MDTDGLPKEQLLVDRPTAMRMLGVRDPRTV | | |
| | | NKLVRNGRITGFTACGRWYENRASIEAFAR | | |
| | | GE | | |
| 13 | 280 | MAKAWIRDRWLKSKAVVDGVELTPTTGMKR | 54.2 | ['Replication, recombination |
| | | QVAANPDTADVPAALRTADYGRGMRWQVCW | | and repair', 'Site-specific |
| | | RADGRQHRESLPTREAAEARAAELNDDIRS | | recombinase XerD (XerD) |
| | | GRYVDPRGGNRTLDEVFPLWLADHTGVRPA | | (PDB:1A0P)', 'Phage |
| | | TIDNYIRHYNGMVRPRFGSTRIGDIDERAI | | integrase family', 'integrase'] |
| | | KAWVADLDSGAIRTKYGEPYTKGAIKTGVR | | |
| | | RLLGSMLRYAVRRRWIMADPTAGVRLPRAP | | |
| | | MQRVDAFTPAEARAIADAAGKLRTPTGRPC | | |
| | | GRPMDRLIVLLLASTGMRPGEMAALDVRDV | | |
| | | DLAHGTINVDKTMTKAEAGHYRYVQGDTKT | | |
| | | PKGRRRLPIPPFLRDGLEELVAGRDGGEPL | | |
| | | FTSPRGERLLYAQWSKRVFRPAMAAAGIDP | | |
| | | DGRALTLYSLRHTFASVAIAAGADVKTLQE | | |
| | | LMGHEDATVTLNTYAAAFADRRVEVAGAVS | | |
| | | DAFDDALGQTPTDGHF | | |
| 14 | 281 | MPNHVDPEDAMRPGKDYTAEQLNGLLALLN | | |
| | | SKYDLRGGDIDTHPVLGGIRFSEPPALDTA | | |
| | | DRWLTDSGPIAEAAKTSTGRPIWPIGYQKA | | |
| | | VTAIVEGRYRCCEDDPDTFYALDRPDDGVE | | |
| | | AMSSYHVLDFAVELEVRTSGRVREIERALK | | |
| | | AMIRLMPRLRPDLYAPVRRGARIHTPSGRQ | | |
| | | AVMYVKDGRPTLVRAQDGSDGVPYIVDVEV | | |
| | | PEEALPGSDKDWRIMCGRVHQWMRFVTKTE | | |
| | | ASAMNLALYWAYPIMAPRMENLWCFYGDGG | | |
| | | NGKGMLMKSFKDVFGRWCGDIDIERLAEGG | | |
| | | FDGGNEAGKLTDALWVEDPESDMSDAKCAR | | |
| | | AMKRIATGDPFTARYGGGVAYEVRPHCGEM | | |
| | | AATNLPPYKEATRAFERRLVEVNAYDGHLP | | |
| | | AEFAPMADWLDNRHGAVELLLTSANLWAGG | | |
| | | YKLDLDESIGTLTGLGEDELFAIKELVEKG | | |
| | | FTVSADNPYLGKGRRYSRSFINRTGLKTGR | | |
| | | RQGQFGLMVKSESMFAPYREAMESMLTADV | | |
| | | RRAEERRADRAANPATDLSTLAPNGMDPVD | | |
| | | WARAKRIGGDTGVYVPAEGKVARDWKRKTE | | |
| | | NGEATPLPDFTKSDSAAEVAGEGMVIIDWD | | |
| | | ADHDGTGDPHGLNRMECDLGAVIGSDDFPM | | |
| | | PYLERSARGGYHGAYRVPSDLMPFFKKSAV | | |
| | | GKATSGDPTLLVDLKAAGGGYVICAGSRTD | | |
| | | QGAYEPVSAPADGKVPMLTQPMLELFSEYG | | |
| | | FIRLPEAYMGPDGKPHVGHGDARPTVAPSA | | |
| | | YARFKSSDGRLHLDTSPMAPHTRHNELVRR | | |
| | | AKYVVAASRAMDLPESAVTEAFDTLRALAG | | |
| | | DHDPSDTERVLRDMANGYGCKYFEPRND | | |
| 15 | 282 | MDGQDELVTIAWIAKRMSIARPSVTKIMRA | | |
| | | MGVPRYEFTSKCVRYPKSAVEAAIAASYRP | | |
| | | ASAGNE | | |
| 16 | 283 | MRRGRTSPSAPNALRRPEKAANTLNRGSLT | | |
| | | ASLQLTMAFAACREWLHSAVFRPEMQLMQL | | |
| | | LQLFAYKKTERHPDDTWGNHRPPCLHRKEI | | |
| | | ANDYINYINYIYCLKMASFRRFRAFPLTAI | | |
| | | RTAATAFLQLTSSSRSRGAQIAFTLVDFGH | | |
| | | AMKAEDRCPSCPPVCLRQPLTSQSGIYPLH | | |
| | | RRSRMIPPFSPSDSLRTHRRRLKVFAASGR | | |
| | | IDA | | |
| 17 | 284 | MSLPPQRQLDNYLSQFAEKQVDGKYLGLYD | | ['Signal transduction |
| | | GERRFGRVFAWLHEQYNGAFEFMNAKAPQG | | mechanisms', 'Protein kinase |
| | | VGGHFNADPSRDLMEVNETYSALLRIASKA | | domain'] |
| | | GIRIKTKPEYQKVIDSSRGWLQPTLGSPIP | | |
| | | EGLTPIEVEYYDTVFETEDSGIMLAGTNRV | | |
| | | PLQFVGEGSYAIVHKFTDPNYGIQFARKKL | | |
| | | KKGVKPKEVERFHREFDIMKRFDFPYILKV | | |
| | | YRYNESDNSYTMEYCEHTLKDYISHNNQKM | | |
| | | SPWARRKMAMQFLYAMNFLHKHGVCHRDLS | | |
| | | YGNVLIHTYDDGAFAVKVSDFGLAKERNSD | | |
| | | LTSMGSSMKGSIEDPALKSFKDFKPVNDIY | | |
| | | SIGFILNYIFTGRRDLLADGSRLGSIIQKC | | |
| | | STTNSADRYQTVKGIIEDMRKTECPVG | | |
| 18 | 285 | MEQEALLAQARKTFHTRLIDWGVLGYTVKK | | ['NgoMIV restriction enzyme'] |
| | | GEPVASNADVSQTVSKAIGNGIMTRVLNES | | |
| | | QQEPRKPLSGQALGAQFEIAVCDFIADTFP | | |
| | | HLGHLRPGEWIIKRLGNRNAVTEKNFAQYE | | |
| | | HLAALGRAVEQDPVLQAALGNDYVVAPDII | | |
| | | VARQPETDQTINAPFAVVNDVVANLSDLRQ | | |
| | | RHEGESQRAPILHASISAKWTMRSDRAQNS | | |
| | | RTEALNLIRNRKGQTPHIVVVTGEPAPSRL | | |
| | | ASLALGTGDIDCVYHAFLPELIASVDELKG | | |
| | | REDLAELIHTLVDGKRLKDIADLPLDLAI | | |
| 19 | 286 | MDALVGLEEVAGLIGAEPRIRQNSPSHDDL | | ['Replication, recombination |
| | | EAKASTDLSQRVIRLAKRLEIVVFSPLCRR | | and repair', 'DNA-cytosine |
| | | FEVTLRYMTKYTDVELCAGAGGQAHGLELA | | methylase (Dcm) |
| | | GFEHVALVEIDRHACETLRLNRPQWNVLEA | | (PDB:3LX6)', 'C-5 cytosine- |
| | | DLHEWDPSAYEGVDLLSGGVPCPPFSVAGK | | specific DNA methylase', |
| | | QLGELDDRELFGRALEIIRQIKPRGVMLEN | | 'DNA (cytosine-5)- |
| | | VRGLLDPKFDTFRKNVSDRLRRYGYSPQWR | | methyltransferase 1 |
| | | LLQASDYGVSQLRPRVICVALRPIDMRHFV | | [EC:2.1.1.37]'] |
| | | WPEPDEATPPTVGELLGDLMAANGWKGAAA | | |
| | | WSKQANTIAPTLVGGSKLHGGADLGPTRAK | | |
| | | RAWMKLGIDGRSIADDNPPADAPNDFTPRL | | |
| | | TVRMAARIQGFDDEWQFSGRKTSSYRQVGN | | |
| | | AFPPAVARRVGQQIIRAWEATDGTGSAIST | | |
| | | GKEDIPHPTD | | |
| 20 | 287 | MTNPQAPFQTEQPATQHTEALVRAEGMLGT | | |
| | | VELHGGSLITIDHEGVAALAAKTRHVEIPV | | |
| | | TAIVDVVLTRPTIMTNGRLDLVVRTTDGDS | | |
| | | RYDPKSPNPFSVSFLAKDLDGERNVAQSIK | | |
| | | AAKPAEPAAIEPPKPEKRRPFWRRGAFWSM | | |
| | | AALAVVFCCLVNSCGSVSDDSATESMPDVV | | |
| | | GMDYAHVHQALADYHDVDYIDLKGNPTLSG | | |
| | | VATKQSPKAGAKVSHSTGITVTLDPQGKTK | | |
| | | EQKEQELSKRVADCKDKDAATVIAGLDADK | | |
| | | LTGTIKTSPNAPTDMADTIRGGIAQGTAYI | | |
| | | VTDAIVDNGKIDLVVDTVAHHNRELASQQI | | |
| | | PAMCEAAGKQSHPYGFKVPLESDNSGVVES | | |
| | | DDTNWSYSFEANITNAFGAVAKGVPITCTG | | |
| | | TLTGQNIVVSGIN | | |
| 21 | 288 | MAGCSAWFRYAVCHHGASFRHGSAGENMAV | | ['Carbohydrate transport and |
| | | FVSHAFVLYYSGALFRYEKYDSILACVKDQ | | metabolism'] |
| | | AKKLLFPYAMLTIVCAPFWYINHIVLGDGK | | |
| | | VSFKRLLVGFLTADQYEGTMTNGALWELPA | | |
| | | LFLTSVVFWFLVDLEHKGKVNLAGSLGICA | | |
| | | ALAIGVGIFCEGKPTAWNVAGVPAMVIFYG | | |
| | | IGCMAMQSYRRHRDAIESLSPTVMTAAIIA | | |
| | | LMSVGTWAALANTKVSLLVNDYEVFALMLV | | |
| | | SSLGISACLALLEMDLPNVRPLTFAGRESG | | |
| | | LSWSAYSYDAFL | | |
| 22 | 289 | MRGLAAALGLVHQLVGVAVVAGAVRYVFVL | | |
| | | ITHDVPDSFGRCGLMVTYAMYQAKLEECYS | | |
| | | LALPKQLRDAVQQTAILLSEGKSVTISATE | | |
| | | QELTTEQAAYTGSIATDASQNVR | | |
| 23 | 290 | MFADALEYVTAQDQPENEIVKNMKHFFQKV | | |
| | | GDNYLKFTDPNSKCIILPNTDENQQIIKTL | | |
| | | CNWKPQLIPQIEKIDEKNLIKIRRDQKSWN | | |
| | | EFFKQGE | | |
| 24 | 291 | MALIADTTEDNEDSLRVGLNANNASDASTP | | |
| | | QAWRELTGCEETDQVYVEYGSRWARTTRLI | | |
| | | PYSLLKRVIDTSLDIGQWEETQKTILINKN | | |
| | | ADLSKTIEDLRSGDWAFILQFEESQTKESQ | | |
| | | TSLGKDFYALLGGNTSLLYRLISHGWIGQD | | |
| | | YMLYSTIYTDEGITRNALNFILHHIDGNNP | | |
| | | DYQFKLDDKDSEDVLRRIRRKDKAIFHQHR | | |
| | | VLNIDLLHYMLSHCDDSETNQDTDQIIEML | | |
| | | SPLGNETAEFVSAYLDRLADDANPLLVKMA | | |
| | | SHSQNLLSFLAQRGTTGTDMLCQHLDVAFH | | |
| | | HLSREIQYTVDGNLADFLQNHWRSISIFTD | | |
| | | SNIAPAFVEPIVQILNDGKVRIDSLSELGY | | |
| | | KDSNSGLRYPCVEQNLYVFSRGNLETALDT | | |
| | | ISQSDNNQDGSYSVCNRLPALNMIMHISRT | | |
| | | VYANVLGHLEEYFDLLTDNEKTLDCPSGQN | | |
| | | IADDLFHILDDICQQAGQEKSAEKLIHTLL | | |
| | | DKCSPSARFPLFEDRFDHFEDRFDHFDVYA | | |
| | | TLMENDAVCFTPINLQFVENRGFDEGNHSL | | |
| | | VEWIQKHDSFEQDSERKEITPEFLRTFGER | | |
| | | P | | |
| 25 | 292 | MTKKSKADSTDNSTNTTDPTSKAAELKPLY | | |
| | | PQYDESSHSPYVKRLEKALQGEDTKIHNIA | | |
| | | LSGVYGSGKSSILEKVVKDLEKERPHTTRT | | |
| | | ISLAPLAAQLKKQDDRKVNSISNEGNSASR | | |
| | | EAPGALPPKSSKPSSITNLIQKEIIKQLLY | | |
| | | GTDPEKIPASHFHRINEIGLGKQLLSSLAC | | |
| | | GTLLLFILDIHKWPYNRIQELLTWLPIPTS | | |
| | | ITKILAEIVIIGLLLIATFALFHYFGTRIH | | |
| | | LAKIDVGTAGITLGENSDSYFDQYLDEIIY | | |
| | | IFEKTGIRTVFFEDLDRFQDAQIFDSLREL | | |
| | | NQILNNDPKLQRTKSTIQKNSRQSHSSHSV | | |
| | | KEKNSQSSTPIQFVYAIHDAIFSNQYVSAA | | |
| | | EQVSNKESLAHSFSRAKFFDLIIPVVPFVS | | |
| | | ASNSCQIASETLEDVLDANDPQMINLLELV | | |
| | | ADAVPDKRTWINIRNEFIVYREHLFVQSPE | | |
| | | GRFTSKLGLEESHLLAFIIYKKLLS | | |
| 26 | 293 | MRKSVEPAGVPTLPEVGSIVEVRGATWAVT | 29.8 | ['Transcription!!!Replication, |
| | | DVRGQGISRSPADESRGNRQHVVSLQSLEE | | recombination and repair', |
| | | DRMGEELSVIWELEVGQTVLPDQGLPDIIN | | 'Superfamily II DNA or RNA |
| | | ADSFDDPNRLGAFVDAMRWGAVTSADAKKF | | helicase, SNF2 family (HepA) |
| | | QAPFRTGANLEPYQLEPLRRALSAPRTNLL | | (PDB:6UXV)', 'Helicase |
| | | LADDVGLGKTIEAGMVVEELLLRHRARTAI | | conserved C-terminal |
| | | IVCPPSLCVKWREELLEKFGLDFVIVNSET | | domain', 'ATP-dependent |
| | | LAESRRKYGLGANPFRLYPRVIVSMAWVPS | | helicase HepA [EC:3.6.4.-]'] |
| | | IRAQRLLEEAYSDTGGMNSARQYSFDILVV | | |
| | | DEAHHVAPAAPASSGRVRGYAVDSKRTETI | | |
| | | RRLAEHCEHRLFLSATPHNGYSESFTALLE | | |
| | | MVDSRRFTRGAAIDEKALEEVTVRRLKTNI | | |
| | | ASLHFKERHIETIPFTPGDDEEQHYAMLVK | | |
| | | ILAKTAKEHSDRDRALGVTALLFKKRLLSS | | |
| | | PWAFARTLSRYRDLSEDDGYDSWMDDDYYS | | |
| | | EVMGSGQSDEEEGREEQPEFETLAKGRKDN | | |
| | | PLAAAEEGQLDELEDWADRFEGRPNSRLEA | | |
| | | LIEWLDSVCRPGGGNVWSDERVVVFTEYAD | | |
| | | TLDWIQRILESRGYTKDCLSVIDGQTDAEE | | |
| | | RELIRARFNANPKDEPIRVLLATDAAGEGI | | |
| | | DLQTYCHRLVNFDVPENPSRLEQRIGRIDR | | |
| | | YGQTQTPEIYYFRPSQKGSLLEGNLEFMNR | | |
| | | LAQKVSVEVEDLKTVNPLIDREISDHFLGG | | |
| | | NGKPQVRAADEMGRQANEIINKTLAGSVAL | | |
| | | NRQLTELADEYETSKRMMHLTEQAEKRVVD | | |
| | | VSLDLTNQPPLITTDDPKVYRLPDLNPGWR | | |
| | | PIEDGLRTVLDPDRVRPITFDADVARHNPD | | |
| | | VAYMHLGSALMSKASRTLRGNLYGQESKLH | | |
| | | RVTAVVVPGLDATCAAAMSRLVLVGRGGLR | | |
| | | VHEEMFVTGLRFRAQNLAEEKVQTLLADAL | | |
| | | DASKTLQLADPKILERLEREWDANNGRLRS | | |
| | | RLEDAIGKRAETRKQLVESNLEERRESDLD | | |
| | | RAKGIFEQFRRNLRDSLAALRRQDEDDAVQ | | |
| | | LSLWEDEEQKQRKRDIRKMEQRLDDLDSEE | | |
| | | SRELDMIRLRYKDVKPYVSIAALVFAVSEQ | | |
| | | DAEQWRAE | | |
| 27 | 294 | MMVRNRRSNQRMRSNSPEQMHREWLELVDT | | ['Defense mechanisms', 'Type |
| | | DGPFLAAPVLKRAWPQGMSLLKTLENGQQR | | I restriction-modification |
| | | AAELRQEKAAFEAAWDEWHRVRVGSDDDEA | | system, DNA methylase |
| | | VADATRKYREQENAWVTFVVRRLLDWREDY | | subunit (HsdM) (PDB:2AR0) |
| | | RLAEDDAAEAAAYDAESPNGAIRVSPSGLL | | (PUBMED:26872910)'] |
| | | ELNGTVGAVVLVVDPVVESLTEIPDDGWNA | | |
| | | SAIDRMQHMLRTKQSTCSIGLVTDGRWWAL | | |
| | | VSAPKGKSAAWGQFDSQMWIDTPQVLNAFV | | |
| | | NLLSIRSLVTDAEEDRLPALFAESVTAAED | | |
| | | ITEALGGQVRQAVELIVAAFNESSARARNA | | |
| | | GRPDPLPDDGERSYEAAVTVMMRVVFLLFA | | |
| | | QERGLLPRSGLFENAYGLAGMLDMLEERAR | | |
| | | DEGEEAMDGTSMVWHRLLATSQALYGGVNF | | |
| | | EDMRLPAYGGSVEDPQRAPFLVATDEHGEL | | |
| | | TVVVSDRVMHHVLRSVQIAVVGRESRRISF | | |
| | | RDIDVEQIGYIYEGLLGYTCLRSKEVVLGL | | |
| | | EGAKSQEPEVPLSVLEQIAADSADDASGAK | | |
| | | RAKAIEEWVKANASASKTPSRSKMGKLLTG | | |
| | | SSPEDVERALLSVTHDKAMQDRLRPWMGLI | | |
| | | RRDLRGKPVVFLAGDLYVTETSSRKDAGAH | | |
| | | YTPRALAEEVVVHALEPLIYQPGPLQTNDR | | |
| | | SQWRHISSTDLLNLHVADIACGSGAFLVAA | | |
| | | ARYLSAELVEAWRLEHALPYDGTPEQTRLK | | |
| | | AIRMVVARCLYGVDINEMAVEMCKLSLWLV | | |
| | | SLDKNQPFSFVDNKILHGNSLLGVTNLAQV | | |
| | | EYKRIDAKPRQQIQLFEINDLNQTASVVDV | | |
| | | SPVIRRVRNIREQLSSEISADDPQRSAVAK | | |
| | | HRQMHEMDEALAQLRKIADGVVAAGLRGIC | | |
| | | ESGKRMDEEYGNLAVAVGRAFPAEGEGDSR | | |
| | | MLDDIIDSGLKPTVPTDYEHWRCVHWDNAR | | |
| | | ILAHFRIPALT | | |
| 28 | 295 | MIQSAQLFGHYLHWPLEIPEVMENGGFDAI | | ['Defense mechanisms', 'Type |
| | | IGNPPFLGGKKLTGTMGENVRKWYINILAG | | I restriction-modification |
| | | GNRGSADLCAYFYLRSYSLMRKGGTLGLLA | | system, DNA methylase |
| | | TNTIAQGDTREVGLDSMTDNGFTIVRSVQS | | subunit (HsdM) (PDB:2AR0) |
| | | KPWPVSSANLEYAAVWGVKGAVSDDVAKDC | | (PUBMED:26872910)'] |
| | | DGMKVSRISTLLEPQGRASGQPDALKDNKD | | |
| | | VSFIGCYVLGKGFIISQEQAAEWIEADLKN | | |
| | | KEVLFPYLNGEDLNSRPDCSASRWVIDEND | | |
| | | WNEEKAKKYALPYKHLLKYVKPERQRKKPD | | |
| | | GSYQLRKSLPERWWQYGDKRPALRKAIFPM | | |
| | | NDVLVIALVSKILMPLRVPTGQIFSHALGV | | |
| | | FATSSYADQAVLSSSIHQYWAISRGSSLES | | |
| | | RLRYTPSDVFETFPRPQNTDALAAIGKTLD | | |
| | | EERREIMMRRQLGLTKLYNLVNDPDISDSS | | |
| | | DADVARLREIHRHLDETVMAAYGWSDVPLD | | |
| | | HGFYEYRKMIRWTVCPEARIEILDRLLEEN | | |
| | | HRRAKLEAAQGGEHE | | |
| 29 | 296 | MSEQTTETANPESKIYELGYPLDGSSYACR | 31.7 | ['Replication, recombination |
| | | ENLQDILRREMMGPSNGENEILEVSPKSKY | | and repair', 'ATP-dependent |
| | | ILGRIAPTKIMDADEIAHHGMRENTDADPN | | helicase YprA, contains C- |
| | | EEPDEDLDDDAESFDDELHDAPQRRGLLIP | | terminal metal-binding |
| | | SSMGMRFQIPDDLESFTVHCSWGRYSPVGT | | DUF1998 domain (YprA)'] |
| | | GKQDKRGNEVRAYQRTPVAHSVKVRLSDLV | | |
| | | NDRTTIRVEDTVELCVDRYDDPALHRCFIE | | |
| | | VALCNDTESEKPIPVSKWLFQTQLDVDADG | | |
| | | KAVFLPVHDWNEDPSFEKEQDFEQKKLRLQ | | |
| | | YRNRLEFAVGRTCSVDWTVSPENSRRAVSV | | |
| | | \|RTTWLPTADIPQTIAQNVEGAELDMTALAV | | |
| | | MGPEELRKALTPISDGYRSWLDEQEASIAA | | |
| | | LPEHLRKTARGTVQLARIVSKQLADGIDEL | | |
| | | CSDEEALRCFHFMNTVMAEQRVHTQVNALR | | |
| | | GTNGDLSLKETEKRVLSGSYPHHWRVFQLA | | |
| | | FILMQIHALTDPAVEVRSDESTKAKTQLLE | | |
| | | FPTGGGKTEAYLGLAAYTFAIRRRQGIVKS | | |
| | | PDGNLNGNEGVAVLMRYTLRLLTSQQFQRA | | |
| | | STLICAAELERRKNPELWGDEPFRIGLWVG | | |
| | | TNVTPKKVSEAAREIEQTRLRASNRNPDVL | | |
| | | QIVSCPWCGKPLGSGDLDVDKVRGRVFVHC | | |
| | | PDMRGECPFAKGGEVTEGIPVLTTDEEIYR | | |
| | | LVPSFVIATVDKFARLAREGAASALFGYVG | | |
| | | RKCDRHGYVPNLDIEENSDYDDCSIKDDSA | | |
| | | HPEKRNCPAAHIHPAMRLRPPDLIIQDELH | | |
| | | LISGALGTTVGLFESAIDVMCTWKDQNGRD | | |
| | | IRPMIVASSATMRNAADQIRKLYGRGVTVF | | |
| | | PPQVLDVSDTFFSKEQQCDEDTPGRRYIGI | | |
| | | STTGVRLSNAEIQTAETLLKGAQRLMNDPK | | |
| | | GGNAADPYMTLVGYFSTIRELAGMARFMQD | | |
| | | DISTHVRRGRLGSHLPRRYGAQFGDLNVGE | | |
| | | LTSRISSTDIVTTLDHMNNTFDEDHDSQQA | | |
| | | WHRNAELRKQGKGTNNRDSADIPFDAVLAT | | |
| | | SMLQVGVDVSRLGLMMIVGQPKNTAEYIQA | | |
| | | SSRVGRDAKRPGLVVTIGNWARPRDLAHFE | | |
| | | QFKAYHDSFYARVEPLSVTPFSVTALEHGV | | |
| | | EGLLVSAVRVMQANQKTGLNPERNAGLAAQ | | |
| | | EHGVLDTIVTRLIERIHIAGGDDAAKEADS | | |
| | | RLRNRMDTWENLARSASGNGRTLVYERAPK | | |
| | | DDSDYQRLIHSAEELGNREALTSRCFVIAN | | |
| | | SMREVQPEINILVSPNPEKLGFTEPDGAPT | | |
| | | WQSQQATNTAKQETEHE | | |
| 30 | 297 | MSDTKASEDGLIYDPQLDVDPLGDQDELEE | 29.0 | |
| | | NNTAKNYAKVGSSRGSTLMYTYGPGSIMDL | | |
| | | PHFTVMPMGLNAWDKIWKRRPGISKITAPR | | |
| | | LLENVQLMLGNQVKELRPFPWQPNQTGAFR | | |
| | | EGADLGVPARMFPQWLRCTGCNKLAPVSDF | | |
| | | VNGYSNTNPYRPDQAEFMHKGCHGSGKGAK | | |
| | | KYDRPCVPARYLLVCEDGHVDEFPYDWWAH | | |
| | | NGGHCPNASKPQLKMIESSAGVSGSFIECV | | |
| | | SCGAKRSMLEAQSLENRSKLPRCRGRFAHL | | |
| | | DCFQAKPCDKPVRLMLIGASNLWFPVVQSI | | |
| | | IDMPRLDEKAVIRDEYNMIKTALGDNDWML | | |
| | | EEDLDDNLKSIRKTVQRSAKTDDELKQKSE | | |
| | | VELHSIILQGQQSEMNEDERQLAREQWEPS | | |
| | | DLLVPEWKYLVRDFPDTKHVDRKSGLTVHV | | |
| | | QQVHGIVADLGVKRILAVDKLKKVNALIGE | | |
| | | TRVDDFDRVNDLGSRLVRLNRDGRPTWVPA | | |
| | | TEDYGEGIFIQFDEERIEKWENAVLANPLW | | |
| | | DSHVMAHRRNFRNRLSETAAVVDPDTRLPK | | |
| | | PRYWLMHTLSHALIKRMAMSAGYGIASLSE | | |
| | | RIYAWQGSDDRPAAAGVLVETTASGSDGTL | | |
| | | GGLVDLSNTDKFEQIMTSALQEMKRCSSDP | | |
| | | VCARRIPKDPEDFLHGAACHCCCMLSETSC | | |
| | | ERANRFLDRRFLVPLPGQDTSLAFFKD | | |
| 31 | 298 | MDKIQTDALIELGASMTGLEASDIANYLED | 43.1 | ['Lipid transport and |
| | | GYPLEEAFSNVQEFHRKRICELFEIAGFGD | | metabolism', |
| | | DDEQQVQETIKELRGIQGAYRDPEQTTAVW | | 'Phosphatidylserine\ |
| | | TSPHGLVREGDLNSSRSHMIEAATSSIVCS | | Vphosphatidylglycerophosphate\ |
| | | TFNFQRSSALWESLKKAATLPGMSVKIYVD | | Vcardiolipin synthase (Cls) |
| | | TSANSADDTTRKGKGPTSPTPEEIAKEIVG | | (PDB:1BYR)', 'Phospholipid |
| | | AKVFCTAKSDKGYYYRNHAKFISVDHQDLL | | biosynthesis'] |
| | | VTSANFSYSAEELNIELGLRIHDEALAESI | | |
| | | ERQMANMESRLYRRVGVREEPDE | | |
| 32 | 299 | MNDSDENPRKRHVVFQKLNITPSSMPIISL | | ['Defense mechanisms', |
| | | IASIKNVRANGLDLSPDYQRGYIWSNEYKD | | 'DNAse\VDNA nickase |
| | | QLILSIILNYPIGNIVINNLDQPNQRNARQ | | specific for phosphorothioated |
| | | ELVDGKQRLTTIFRFMEVGNVGQWLDSYDD | | or glycosylated phage DNA, |
| | | WFQLSKKTSDQAKEIINRIVGDSDPDGLAR | | GmrSD\VDndB\VSspE family, |
| | | MHRAKRLAFSDLPSSIQMNENTYNIPVYTM | | contains DUF262 and HNH |
| | | QAADPAQIRNYFKVLQNQEKLRAGEIINAL | | nuclease domains (GmrSD) |
| | | PDNPMSMYFDRIPAEAFLTRTGCSNFKRAE | | (PDB:6JIV) |
| | | LEKVYYSVLGTWFDKIQINASDKTVISFVE | | (PUBMED:17188297;32251370)', |
| | | NMPELTEAQIEHINNLNSGIIAISRLPGAV | | 'Protein of unknown |
| | | QKIRSSKRMLKLVFGLALHAPGYESTTDAF | | function DUF262'] |
| | | SRLQSVCELSSKLAAFNTSDSDQVAFSKYF | | |
| | | GDEYTLDKENFETRKACVYRALFWSTSRVS | | |
| | | SRTAYVDAMEILRRMFTESFDSAFEYYTAH | | |
| | | NIAK | | |

### HMO Utilization Genes

HMO utilization genes were detected in PB-STR-321 using a functional genomics pipeline built with CENTRIFUGE™ (Kim et al. (2016) Genome Research 26:1721-1729). The pipeline annotates a genome with functional annotations including KEGG ORTHOLOGY™ database (KO) numbers. Lists of KO numbers associated with HMO utilization genes and HMO utilization gene clusters were obtained from published research (Henrick et al. (2021) Cell 184: P3884-3898). The Blon gene accession IDs associated with each observed KO value in PB-STR-321 are listed in Table 27 where the genes are grouped by the HMO utilization gene clusters from Henrick et al.:

TABLE 27

| | Number of | |
|---|---|---|
| Cluster | Genes | BLON IDs |
| H1 | 7 | Blon_2331, Blon_2332, Blon_2334, Blon_2336, Blon_2348, |
| | | Blon_2357, Blon_2360 |
| H2 | 1 | Blon_0248 |
| H3 | 2 | Blon_0423, Blon_0426 |
| H4 | 6 | Blon_0641, Blon_0644, Blon_0646, Blon_0647, Blon_0648, |
| | | Blon_0650 |
| H5 | 7 | Blon_2171, Blon_2172, Blon_2173, Blon_2174, Blon_2175, |
| | | Blon_2176, Blon_2177 |
| Urease | 2 | Blon_0108, Blon_0115 |

### Bacteriocins

Using ANTISMASH™ (Blin et al. (2023) Nucleic Acids Research 51: W46-W50) bacterial version, the PB-STR-321 genome was searched for bacteriocins, peptides known to have antimicrobial and immunological properties relevant to the infant gut environment (Benítez-chao D. et al. (2021) Frontiers in Microbiology 12), but no bacteriocin signatures were observed.

### Antimicrobial Resistance Genes

The ORFs found in the genome for strain PB-STR-321 were BLAST searched against the NCBI Antimicrobial Resistance Database and no antimicrobial resistance genes were observed. Antimicrobial resistance genes are increasingly common in infants (Shan Y. et al. (2019) Nature 574:117-121) and pose potential health issues (Samarra A. et al. (2023) Gut Microbes 15:2194797).

### Vindence Factors

The ORFs found in the genome for strain PB-STR-321 were BLAST searched against the VFDB (Virulence Factor Database) and no virulence genes were observed.

### Biotherapeutic Combinations

Based on the strain definitions provided above, a new set of biotherapeutic combinations was generated with strain level resolution (Table 30).

TABLE 30
List of exemplary live biotherapeutic combinations, mixes or consortia,
or probiotics with strain level resolution as provided herein.

| | |
|---|---|
| 1 | PB-STR-093 |
| 2 | PB-STR-207 |
| 3 | PB-STR-083 |
| 4 | PB-STR-119 |
| 5 | PB-STR-093 |
| | PB-STR-207 |
| 6 | PB-STR-093 |
| | PB-STR-083 |
| 7 | PB-STR-093 |
| | PB-STR-119 |
| 8 | PB-STR-093 |
| | Bifidobacterium infantis |
| 9 | PB-STR-093 |
| | Bifidobacterium longum |
| 10 | PB-STR-093 |
| | Bifidobacterium breve |
| 11 | PB-STR-093 |
| | Bifidobacterium bifidum |
| 12 | PB-STR-207 |
| | PB-STR-083 |
| 13 | PB-STR-207 |
| | PB-STR-119 |
| 14 | PB-STR-207 |
| | Bifidobacterium infantis |
| 15 | PB-STR-207 |
| | Bifidobacterium longum |
| 16 | PB-STR-207 |
| | Bifidobacterium breve |
| 17 | PB-STR-207 |
| | Bifidobacterium bifidum |
| 18 | PB-STR-083 |
| | PB-STR-119 |
| 19 | PB-STR-083 |
| | Bifidobacterium infantis |
| 20 | PB-STR-083 |
| | Bifidobacterium longum |
| 21 | PB-STR-083 |
| | Bifidobacterium breve |
| 22 | PB-STR-083 |
| | Bifidobacterium bifidum |
| 23 | PB-STR-119 |
| | Bifidobacterium infantis |
| 24 | PB-STR-119 |
| | Bifidobacterium longum |
| 25 | PB-STR-119 |
| | Bifidobacterium breve |
| 26 | PB-STR-119 |
| | Bifidobacterium bifidum |
| 27 | PB-STR-093 |
| | PB-STR-207 |
| | PB-STR-083 |
| 28 | PB-STR-093 |
| | PB-STR-119 |
| | PB-STR-083 |
| 29 | PB-STR-093 |
| | PB-STR-207 |
| | PB-STR-119 |
| 30 | PB-STR-093 |
| | Bifidobacterium longum |
| | Bifidobacterium infantis |
| 31 | PB-STR-093 |
| | Bifidobacterium breve |
| | Bifidobacterium infantis |
| 32 | PB-STR-093 |
| | Bifidobacterium breve |
| | Bifidobacterium longum |
| 33 | PB-STR-093 |
| | Bifidobacterium bifidum |
| | Bifidobacterium infantis |
| 34 | PB-STR-093 |
| | Bifidobacterium breve |
| | Bifidobacterium bifidum |
| 35 | PB-STR-093 |
| | Bifidobacterium bifidum |
| | Bifidobacterium longum |
| 36 | PB-STR-207 |
| | PB-STR-119 |
| | PB-STR-083 |
| 37 | PB-STR-207 |
| | Bifidobacterium longum |
| | Bifidobacterium infantis |
| 38 | PB-STR-207 |
| | Bifidobacterium breve |
| | Bifidobacterium infantis |
| 39 | PB-STR-207 |
| | Bifidobacterium breve |
| | Bifidobacterium longum |
| 40 | PB-STR-207 |
| | Bifidobacterium bifidum |
| | Bifidobacterium infantis |
| 41 | PB-STR-207 |
| | Bifidobacterium breve |
| | Bifidobacterium bifidum |
| 42 | PB-STR-207 |
| | Bifidobacterium bifidum |
| | Bifidobacterium longum |
| 43 | PB-STR-119 |
| | Bifidobacterium longum |
| | Bifidobacterium infantis |
| 44 | PB-STR-119 |
| | Bifidobacterium breve |
| | Bifidobacterium infantis |
| 45 | PB-STR-119 |
| | Bifidobacterium breve |
| | Bifidobacterium longum |
| 46 | PB-STR-119 |
| | Bifidobacterium bifidum |
| | Bifidobacterium infantis |
| 47 | PB-STR-119 |
| | Bifidobacterium breve |
| | Bifidobacterium bifidum |
| 48 | PB-STR-119 |
| | Bifidobacterium bifidum |
| | Bifidobacterium longum |
| 49 | PB-STR-083 |
| | Bifidobacterium longum |
| | Bifidobacterium infantis |
| 50 | PB-STR-083 |
| | Bifidobacterium breve |
| | Bifidobacterium infantis |
| 51 | PB-STR-083 |
| | Bifidobacterium breve |
| | Bifidobacterium longum |
| 52 | PB-STR-083 |
| | Bifidobacterium bifidum |
| | Bifidobacterium infantis |
| 53 | PB-STR-083 |
| | Bifidobacterium breve |
| | Bifidobacterium bifidum |
| 54 | PB-STR-083 |
| | Bifidobacterium bifidum |
| | Bifidobacterium longum |
| 55 | PB-STR-093 |
| | PB-STR-207 |
| | PB-STR-119 |
| | PB-STR-083 |
| 56 | PB-STR-093 |
| | Bifidobacterium longum |
| | Bifidobacterium breve |
| | Bifidobacterium infantis |
| 57 | PB-STR-093 |
| | Bifidobacterium bifidum |
| | Bifidobacterium breve |
| | Bifidobacterium infantis |
| 58 | PB-STR-093 |
| | Bifidobacterium longum |
| | Bifidobacterium bifidum |
| | Bifidobacterium infantis |
| 59 | PB-STR-093 |
| | Bifidobacterium longum |
| | Bifidobacterium breve |
| | Bifidobacterium bifidum |
| 60 | PB-STR-207 |
| | Bifidobacterium longum |
| | Bifidobacterium breve |
| | Bifidobacterium infantis |
| 61 | PB-STR-207 |
| | Bifidobacterium bifidum |
| | Bifidobacterium breve |
| | Bifidobacterium infantis |
| 62 | PB-STR-207 |
| | Bifidobacterium longum |
| | Bifidobacterium bifidum |
| | Bifidobacterium infantis |
| 63 | PB-STR-207 |
| | Bifidobacterium longum |
| | Bifidobacterium breve |
| | Bifidobacterium bifidum |
| 64 | PB-STR-119 |
| | Bifidobacterium longum |
| | Bifidobacterium breve |
| | Bifidobacterium infantis |
| 65 | PB-STR-119 |
| | Bifidobacterium bifidum |
| | Bifidobacterium breve |
| | Bifidobacterium infantis |
| 66 | PB-STR-119 |
| | Bifidobacterium longum |
| | Bifidobacterium bifidum |
| | Bifidobacterium infantis |
| 67 | PB-STR-119 |
| | Bifidobacterium longum |
| | Bifidobacterium breve |
| | Bifidobacterium bifidum |
| 68 | PB-STR-083 |
| | Bifidobacterium longum |
| | Bifidobacterium breve |
| | Bifidobacterium infantis |
| 69 | PB-STR-083 |
| | Bifidobacterium bifidum |
| | Bifidobacterium breve |
| | Bifidobacterium infantis |
| 70 | PB-STR-083 |
| | Bifidobacterium longum |
| | Bifidobacterium bifidum |
| | Bifidobacterium infantis |
| 71 | PB-STR-083 |
| | Bifidobacterium longum |
| | Bifidobacterium breve |
| | Bifidobacterium bifidum |
| 72 | PB-STR-093 |
| | PB-STR-207 |
| | PB-STR-119 |
| | PB-STR-083 |
| | Bifidobacterium bifidum |
| 73 | PB-STR-093 |
| | PB-STR-207 |
| | PB-STR-119 |
| | PB-STR-083 |
| | Bifidobacterium longum |
| 74 | PB-STR-093 |
| | PB-STR-207 |
| | PB-STR-119 |
| | PB-STR-083 |
| | Bifidobacterium breve |
| 75 | PB-STR-093 |
| | PB-STR-207 |
| | PB-STR-119 |
| | PB-STR-083 |
| | Bifidobacterium infantis |
| 76 | PB-STR-093 |
| | PB-STR-215 |
| | PB-STR-083 |
| | PB-STR-119 |
| 77 | PB-STR-093 |
| | PB-STR-220 |
| | PB-STR-083 |
| | PB-STR-119 |
| 78 | PB-STR-093 |
| | PB-STR-220 |
| | PB-STR-083 |
| | Bifidobacterium breve |
| 79 | PB-STR-093 |
| | PB-STR-215 |
| | PB-STR-083 |
| | Bifidobacterium breve |
| 80 | PB-STR-093 |
| | PB-STR-207 |
| | PB-STR-083 |
| | Bifidobacterium breve |
| 81 | PB-STR-093 |
| | PB-STR-083 |
| 82 | PB-STR-083 |
| | Bifidobacterium breve |
| 83 | PB-STR-083 |
| | PB-STR-119 |
| 84 | PB-STR-093 |
| | Bifidobacterium breve |
| 85 | PB-STR-093 |
| | PB-STR-119 |
| 86 | PB-STR-207 |
| | Bifidobacterium breve |
| 87 | PB-STR-207 |
| | PB-STR-119 |
| 88 | PB-STR-215 |
| | Bifidobacterium breve |
| 89 | PB-STR-215 |
| | PB-STR-119 |
| 90 | PB-STR-220 |
| | Bifidobacterium breve |
| 91 | PB-STR-220 |
| | PB-STR-119 |
| 92 | PB-STR-083 |
| | PB-STR-207 |
| 93 | PB-STR-083 |
| | PB-STR-215 |
| 94 | PB-STR-083 |
| | PB-STR-220 |
| 95 | PB-STR-093 |
| | PB-STR-207 |
| 96 | PB-STR-093 |
| | PB-STR-215 |
| 97 | PB-STR-093 |
| | PB-STR-220 |
| 98 | PB-STR-093 |
| | PB-STR-083 |
| | PB-STR-220 |
| 99 | PB-STR-093 |
| | PB-STR-083 |
| | PB-STR-207 |
| 100 | PB-STR-093 |
| | PB-STR-083 |
| | PB-STR-215 |
| 101 | PB-STR-093 |
| | PB-STR-083 |
| | PB-STR-119 |
| 102 | PB-STR-093 |
| | PB-STR-083 |
| | Bifidobacterium breve |
| 103 | PB-STR-093 |
| | PB-STR-207 |
| | PB-STR-119 |
| | PB-STR-083 |
| | PB-STR-231 |
| 104 | PB-STR-093 |
| | PB-STR-207 |
| | PB-STR-119 |
| | PB-STR-083 |
| | Lacticaseibacillus |
| | rhamnosus |
| 105 | PB-STR-093 |
| | PB-STR-207 |
| | PB-STR-119 |
| | PB-STR-083 |
| | Lacticaseibacillus |
| | paracasei |
| 106 | PB-STR-093 |
| | PB-STR-207 |
| | PB-STR-119 |
| | PB-STR-083 |
| | Limosilactobacillus reuteri |
| 107 | PB-STR-093 |
| | PB-STR-207 |
| | PB-STR-119 |
| | PB-STR-083 |
| | Bifidobacterium |
| | adolescentis |
| 105 | PB-STR-083 |
| | Bifidobacterium longum |
| | Bifidobacterium breve |
| | Bifidobacterium infants |
| | Lacticaseibacillus |
| | paracasei |
| 106 | PB-STR-093 |
| | Bifidobacterium longum |
| | Bifidobacterium breve |
| | Bifidobacterium infants |
| | Limosilactobacillus reuteri |
| 107 | PB-STR-093 |
| | Bifidobacterium longum |
| | Bifidobacterium breve |
| | Bifidobacterium infants |
| | Bifidobacterium |
| | adolescentis |
| 108 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| 109 | PB-STR-207 |
| | PB-STR-215 |
| | PB-STR-220 |
| 110 | PB-STR-003 |
| | PB-STR-093 |
| | PB-STR-034 |
| | PB-STR-035 |
| | PB-STR-083 |
| 111 | PB-STR-119 |
| | PB-STR-103 |

Antibiotic Resistance Characterization of Isolated Strains from Fecal Matter

In alternative embodiments the complete genome sequence of each organism is screened to ensure it contains no genes or pathogenicity island gene clusters encoding known virulence factors, toxins, or antibiotic resistance functions, using publicly available databases such as DBETH55 (for example, see Chakraborty A, et al. (2012) Nucleic Acids Res. 40:615-620) and VFDB56 (Chen L, et al. (2005) Nucleic Acids Res. 33:325-328). Each organism is evaluated by standard antibiotic sensitivity profile techniques such as broth microdilution susceptibility panels or plate-based methods such as disk diffusion method and antimicrobial gradient method (James H. Jorgensen and Mary Jane Ferraro 2009 Clinical Infectious Diseases 49:1749-1755). Such tests determine the minimal inhibitory concentration (MIC) of an antibiotic on microbial growth. Antibiotics evaluated include but are not limited to amoxicillin, amoxicillin/clavulanic acid, carbapenem, methicillin, ampicillin, gentamicin, metronidazole, vancomycin, and neomycin. MIC determinations of novel microbes are compared to published values for both sensitive and resistant related strains to make an assessment on sensitivity (CLSI Guideline M45: Methods for Antimicrobial Dilution and Disk Susceptibility Testing of Infrequently Isolated or Fastidious Bacteria. Wayne, PA; 2015) to determine possible relative increases in antibiotic resistance.

Bifidobacterium infantis strain PB-STR-093 was evaluated for antibiotic susceptibility, and the results are presented in Table 5.

TABLE 5
Evaluation of strain PB-STR-093 for Antibiotic
Susceptibility based on the EFSA standards:

| | Expected Antibiotic | Observed Antibiotic |
|---|---|---|
| Antibiotic | MIC (mg/L) | MIC (mg/L) |
| Ampicillin | 2 | 2 |
| Vancomycin | 2 | 0.5 |
| Gentamicin | 64 | 64 |
| Streptomycin | 128 | 32 |
| Erythromycin | 1 | 1 |
| Clindamycin | 1 | 0.125 |
| Tetracycline | 8 | 4 |
| Chloramphenicol | 4 | 4 |

### Example 6: Growth and Characterization of Isolated Strains and Strain Consortia

### Experimental Evaluation of Metabolism

The growth of isolated strains is evaluated to determine their ability to consume carbon sources and produce specific metabolites. Strains are grown in a minimal media (for example 2 grams (2 ml fine) peptone water (peptone, 10 g/L sodium chloride, 5 g/L), 2 grams yeast extract, 2 grams NaHCO₃, 0.1 g NaCl, 0.04 g K2HPO4, 0.04 g KH2PO4, 0.01 g MgSO4·7H2O, 0.01 g CaCl2·6H2O, 2 ml Tween 80, 10 g carbohydrate of choice, 10 μl vitamin K, 0.5 g cysteine, 0.5 g bile salts) that affords control over carbon source. Examples of carbon sources evaluated (independently or in combination) include but are not limited to glucose, lactose, galactose, fructose, xylose, galactooligosaccharides, fructooligosaccharides, xylooligosaccharides, lacto-N-tetraose, lacto-N-neotetraose, 2′-fucosyllactose, 3-fucosyllactose, 3′-sialyllactose, and 6′-sialyllactose. Growth is evaluated by determination of the OD₆₀₀, which can be performed either on individual samples with a cuvette or tube-based spectrometer (for example the CO 75000™ colorimeter) or on multiple samples in a plate-based format using a plate reader (for example CYTATION 3™). In both cases, values are normalized based on signals from media alone.

Metabolite production is evaluated in addition to growth by spinning down cell cultures and isolating the supernatant for evaluation. The same panel used in Example 3 is used to evaluate metabolite production in the supernatants. Additionally, human milk oligosaccharide consumption (lacto-N-tetraose, lacto-N-neotetraose, 2′-fucosyllactose, 3-fucosyllactose, and 3′-sialyllactose) is evaluated when appropriate.

In addition to evaluation of isolated strains, consortia of strains are also evaluated for both growth and metabolite production. The same techniques are used for evaluation of growth and metabolism, and when necessary, whole genome sequencing (see Example 3) is used to determine compositional information.

Combined information about growth and metabolism is used to determine bacteria and consortia with key functional features including but not limited to human milk oligosaccharide consumption (lacto-N-tetraose, lacto-N-neotetraose, 2′-fucosyllactose, 3-fucosyllactose, 3′-sialyllactose, 6′-sialyllactose), glycan degradation, short chain fatty acid production, and tryptophan metabolism.

### Application to Isolated Strain Library

Select bacteria were evaluated for their ability to consume different carbon sources to aid in selection of live biotherapeutic (probiotic) bacteria and combinations, mixes and consortia of bacteria as provided herein, and prebiotic combinations (see Table 6) used in compositions and methods as provided herein;

TABLE 6
OD(600) after 24 hours growth of isolated microbial strains
on medium containing different carbon sources.

| | No | | | | | | | | |
|---|---|---|---|---|---|---|---|---|---|
| | Carbohydrate | Glucose | Mucin | LNT | 2′-FL | 3′-SL | GOS | FOS | XOS |
| Bifidobacterium | 0.091 | 0.297 | 0.094 | 0.086 | 0.186 | 0.219 | 0.659 | 0.472 | 0.301 |
| adolescentis | | | | | | | | | |
| Bifidobacterium | 0.058 | 0.071 | 0.223 | 0.48 | 0.129 | 0.083 | 0.068 | 0.083 | 0.064 |
| bifidum | | | | | | | | | |
| Bifidobacterium | 0.066 | 0.092 | 0.116 | 0.703 | 0.074 | 0.114 | 0.8 | 0.872 | 0.129 |
| catenulatum | | | | | | | | | |
| Bifidobacterium | 0.062 | 0.076 | 0.248 | 0.936 | 0.133 | 0.146 | 0.068 | 0.092 | 0.065 |
| catenulatum | | | | | | | | | |
| Bifidobacterium | 0.077 | 0.244 | 0.094 | 0.149 | 0.065 | 0.085 | 0.341 | 0.347 | 0.088 |
| longum | | | | | | | | | |
| Bifidobacterium | 0.055 | 0.258 | 0.134 | 0.183 | 0.072 | 0.521 | 1.134 | 0.378 | 0.511 |
| longum | | | | | | | | | |
| Bifidobacterium sp. | 0.083 | 0.519 | 0.143 | 0.224 | 0.138 | 0.303 | 0.936 | 0.832 | 0.153 |
| N5G01 | | | | | | | | | |
| Bifidobacterium | 0.048 | 0.474 | 0.094 | 0.479 | 0.473 | 0.572 | 0.22 | 0.242 | 0.059 |
| infantis | | | | | | | | | |
| Bifidobacterium | 0.055 | 0.146 | 0.236 | 0.743 | 0.118 | 0.141 | 0.129 | 0.077 | 0.058 |
| bifidum | | | | | | | | | |
| Bifidobacterium | 0.054 | 0.392 | 0.106 | 0.104 | 0.063 | 0.154 | 0.506 | 0.093 | 0.165 |
| longum | | | | | | | | | |
| Bifidobacterium | 0.06 | 0.34 | 0.113 | 0.427 | 0.069 | 0.134 | 0.334 | 0.413 | 0.15 |
| breve | | | | | | | | | |
| Bifidobcaterium | 0.077 | 0.822 | 0.08 | 0.201 | 0.101 | 0.134 | 0.408 | 0.467 | 0.065 |
| infantis | | | | | | | | | |
| Bifidobacterium | 0.059 | 0.066 | 0.095 | 0.58 | 0.463 | 0.186 | 0.904 | 0.895 | 0.063 |
| infantis | | | | | | | | | |
| Bifidobacterium | 0.06 | 0.075 | 0.108 | 0.428 | 0.16 | 0.124 | 0.569 | 0.068 | 0.058 |
| infantis | | | | | | | | | |
| Bifidobacterium | 0.055 | 0.077 | 0.11 | 0.054 | 0.055 | 0.057 | 0.805 | 0.064 | 0.051 |
| infantis | | | | | | | | | |
| Bifidobacterium | 0.055 | 0.283 | 0.1 | 0.198 | 0.059 | 0.147 | 0.194 | 0.064 | 0.058 |
| infantis | | | | | | | | | |
| Bifidobacterium | 0.06 | 0.507 | 0.086 | 0.368 | 0.068 | 0.117 | 0.515 | 0.421 | 0.064 |
| breve | | | | | | | | | |
| Bifidobacterium | 0.053 | 0.415 | 0.08 | 0.345 | 0.069 | 0.148 | 0.405 | 0.16 | 0.061 |
| bifidum | | | | | | | | | |
| Bifidobacterium | 0.064 | 0.192 | 0.178 | 0.833 | 0.068 | 0.199 | 0.141 | 0.086 | 0.066 |
| bifidum | | | | | | | | | |
| Bifidobacterium | 0.06 | 0.27 | 0.088 | 0.427 | 0.077 | 0.134 | 0.484 | 0.384 | 0.071 |
| breve | | | | | | | | | |
| Bifidobacterium | 0.056 | 0.106 | 0.078 | 0.065 | 0.06 | 0.058 | 0.11 | 0.066 | 0.061 |
| longum | | | | | | | | | |
| Lacticaseibacillus | 0.124 | 1.187 | 0.138 | 0.117 | 0.106 | 0.318 | 0.273 | 0.263 | 0.252 |
| rhamnosus | | | | | | | | | |
| Lacticaseibacilluss | 0.089 | 0.561 | 0.107 | 0.099 | 0.124 | 0.118 | 0.158 | 0.184 | 0.085 |
| paracasei | | | | | | | | | |
| Bifidobacterium | 0.053 | 0.092 | 0.096 | 0.403 | 0.366 | 0.116 | 0.413 | 0.325 | 0.059 |
| longum | | | | | | | | | |
| Lacticaseibacillus | 0.091 | 0.42 | 0.121 | 0.102 | 0.093 | 0.125 | 0.223 | 0.107 | 0.091 |
| rhamnosus | | | | | | | | | |
| Bifidobacterium | 0.061 | 0.503 | 0.089 | 0.711 | 0.067 | 0.112 | 0.643 | 0.462 | 0.07 |
| breve | | | | | | | | | |
| Bifidobacterium | 0.06 | 0.25 | 0.085 | 0.639 | 0.061 | 0.106 | 0.886 | 0.695 | 0.062 |
| breve | | | | | | | | | |
| Bifidobacterium | 0.068 | 0.335 | 0.084 | 0.44 | 0.064 | 0.115 | 0.388 | 0.36 | 0.067 |
| breve | | | | | | | | | |
| Bifidobacterium | 0.059 | 0.11 | 0.081 | 0.38 | 0.2 | 0.217 | 0.339 | 0.087 | 0.061 |
| infantis | | | | | | | | | |
| Bacteroides | 0.127 | 0.323 | 0.206 | 0.15 | 0.163 | 0.163 | 0.296 | 0.436 | 0.319 |
| ovatus | | | | | | | | | |
| Bacteroides | 0.078 | 0.396 | 0.141 | 0.133 | 0.092 | 0.13 | 0.305 | 0.311 | 0.337 |
| stercoris | | | | | | | | | |
| Bacteroides | 0.093 | 0.251 | 0.231 | 0.205 | 0.237 | 0.169 | 0.271 | 0.387 | 0.256 |
| thetaiotaomicron | | | | | | | | | |
| Bacteroides | 0.071 | 0.188 | 0.105 | 0.142 | 0.133 | 0.12 | 0.229 | 0.246 | 0.3 |
| uniformis | | | | | | | | | |
| Phocaeicola | 0.093 | 0.335 | 0.145 | 0.345 | 0.281 | 0.293 | 0.293 | 0.409 | 0.363 |
| vulgatus | | | | | | | | | |
| Bacteroides | 0.073 | 0.353 | 0.186 | 0.337 | 0.261 | 0.457 | 0.325 | 0.433 | 0.359 |
| fragilis | | | | | | | | | |
| Akkermansia | 0.137 | 0.259 | 0.161 | 0.118 | 0.114 | 0.123 | 0.261 | 0.165 | 0.153 |
| muciniphila | | | | | | | | | |
| Erysipelotrichaceae | 0.091 | 0.406 | 0.155 | 0.35 | 0.44 | 0.307 | 0.265 | 0.448 | 0.362 |
| bacterium | | | | | | | | | |
| Erysipelotrichaceae | 0.064 | 0.055 | 0.097 | 0.061 | 0.064 | 0.158 | 0.063 | 0.058 | 0.063 |
| bacterium | | | | | | | | | |
| Blank | 0.045 | 0.05 | 0.08 | 0.044 | 0.042 | 0.053 | 0.047 | 0.057 | 0.048 |

### Example 7: Immune Activity of Live Biotherapeutic Microbes

### Cytokine Production and Analysis in PBMCs for Isolated Strains

Peripheral blood mononuclear cells (PBMCs) are isolated from human blood using a standard kit and stored in liquid nitrogen at 1×10⁶ cells per mL until use. Prior to storage, PBMC's may be processed using flow sorting or an antibody spin separation kit to select for a certain purified lymphocyte subpopulation, such as T cells.

For use, PBMCs are thawed at 37° C. and then transferred to a growth medium consisting of RPMI-1640 (Lonza, Switzerland), with 10% heat inactivated FCS added, as well as 0.1% penicillin-streptavidin, 1% L-glutamine, and DNase at 10 mg/mL to inhibit aggregation, or a comparable media. Cells are centrifuged at 200×g for 15 minutes and then counted using trypan blue and spread into 24 well plates at 1×10⁶ cells per well (1 mL per well) (Kechaou et al. (2013) Applied and Environmental Microbiology 79:1491-1499; Martín et al. (2017) Frontiers in Microbiology 8:1226) or 96 well plates at 250,000 cells per well.

For evaluation of a single bacteria, an overnight bacterial culture is inoculated using a pre-stocked isolated bacterial strain. This strain is grown at 37° C. for 10 to 20 hours in actinomyces veggie broth medium with added cellobiose (1 mg/mL), maltose (1 mg/mL) and cysteine (0.5 mg/mL) in an anaerobic chamber filled with 85% nitrogen, 10% carbon dioxide, and 5% hydrogen (Martín et al., 2017). Other growth mediums, such as those outlined in Example 1, may be used instead. For evaluation of a consortia rather than a single strain (such as those identified in Table 2, Example 4 or Table 30, Example 5), an overnight bacterial culture for each included strain is inoculated using a pre-stocked isolated bacterial strain. The next day, the consortia bacteria are combined at the desired ratio (based on CFU, OD, or some other quantification method) and allowed to grow together.

At the end of the anaerobic culture, the culture supernatant and bacterial cells alone are saved for co-culture with PBMCs. Microbial culture supernatant is saved directly after centrifugation at −80° C. Cells are saved by washing with phosphate buffered saline (PBS) and then storing in PBS with 15% glycerol. Bacteria are quantified using phase contrast microscopy and stored at a final concentration of 105 or 106 cells per mL (Haller et al. (2000) Infection and Immunity 68; Rossi et al. (2015) Scientific Reports 6:18507) at −80° C. Bacteria may also be pasteurized prior to storage by treatment at 70° C. for 30 minutes (Plovier et al. (2017) Nature Medicine 23:107-113).

Prior to culture with PBMCs, bacterial supernatant is thawed on ice and diluted at a ratio of 1:5 in PBMC growth medium. Microbial growth medium is used as a negative control. This supplemented PBMC growth medium is added 1:1 in each well with PBMCs, resulting in a final 10% dilution level of microbial culture supernatant. Each combination of PBMCs and supernatant is performed in duplicate or triplicate. If bacteria are being evaluated instead, prior to co-culture, bacteria are thawed on ice and then washed at 4° C. with PBMC growth medium. The bacterial suspension in PBMC growth medium is added 1:1 with the 1 mL of PBMC culture in each well of the plate, resulting in a final 2 mL culture containing 1×10⁶ PBMCs and 1×10⁵ or 1×10⁶ (potentially pasteurized) bacteria. The co-culture of PBMCs and supernatant or purified bacteria is incubated for a time ranging from 2 to 48 hours at 37° C. in 10% carbon dioxide.

After co-culture, the supernatant is harvested and directly analyzed or treated with a protease inhibitor (Complete EDTA-Free protease inhibitor, Roche Applied Bioscience) to protect cytokines and stored directly at −80° C. for cytokine profiling. The pelleted cells are treated with RNALATER™ (Thermo Fisher, USA) and saved for RNA sequencing. Cytokine analysis is performed on saved co-culture supernatant using ELISA, a LUMINEX™ system, a Meso Scale Discovery system, or a comparable analytical method. Cytokines measured may include but are not limited to, IL-10, IL-2, and IFN-gamma. RNA sequencing is performed on PBMCs saved in RNALATER™ post co-culture. Standard pseudo-alignment is performed using Kallisto (Bray et al. (2016) Nature Biotechnology 34:525-527) and differential expression is analyzed using DESeq2 (Love et al. (2014) Genome Biology 15:550) to identify differential expression between different microbes and different PBMC donors. Statistical analyses are performed to identify microbes that exhibit desired immunomodulatory effects in vitro, which include but are not limited to inducing production of IFN-gamma and lowering expression of genes associated with T cell exhaustion (PD1, CTLA4, VISTA, TIM3, TIGIT, LAG3).

In addition to evaluation of cellular supernatants as described, different matrices are also evaluated for their immunostimulatory properties such as supernatants isolated from a simulated gut environment like that described in Example 8.

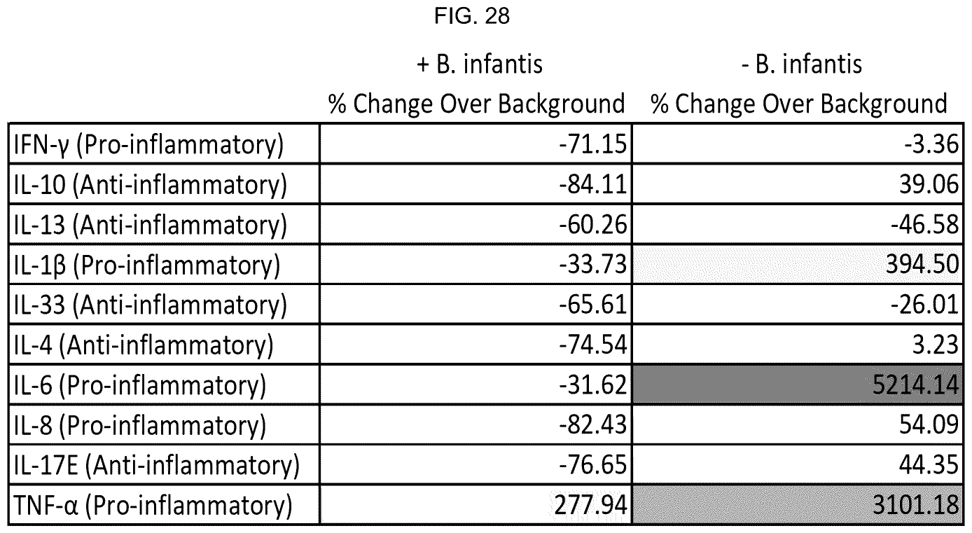

In one example, simulated gut environments supplemented with Bifidobacterium infantis were evaluated for their ability to induce differential cytokine expression when compared to simulated gut environments that were not supplemented. When performed in a C3 environment, substantial shifts were observed, with supplementation of Bifidobacterium infantis greatly ameliorating inflammation that was observed in the C3 gut environment alone (FIG. 28).

### Cytokine Production in Immature Dendritic Cells Induced by Live Biotherapeutic (or Probiotic) Compositions

Single bacterial strains, consortia of bacterial strains, and bacterial strains in the context of a simulated gut environment are evaluated alone and in combination with LPS on cytokine production in immature dendritic cells. A monocyte population is isolated from peripheral blood mononuclear cells (PBMCs). The monocyte cells are subsequently differentiated into immature dendritic cells. The immature dendritic cells are plated out at 200,000 cells/well and incubated with the live biotherapeutic composition at a final concentration of 10⁷/ml in RPMI media, with the optional addition of LPS at a final concentration of 100 ng/ml. Alternatively, the bacterial cells are centrifuged, and the resulting supernatant is added to the dendritic cell preparation. The negative control involves incubating the cells with RPMI media alone and positive controls incubating the cells with LPS at a final concentration of 100 ng/ml. The cytokine content of the cells is then analyzed.

### Example 8: Evaluation of Live Biotherapeutic (or Probiotic) Candidate Strains in a Simulated Gut Environment

### Experimental Evaluation of Strains in a Simulated Gut Environment

To understand the applicability of in vitro observations to the human gut, strains are evaluated in the context of a simulated gut environment. These gut environments are produced with minimal media and seeded with FMT aliquots (Example 2) to reproduce an environment that represents the gut. In these environments, the introduction of prebiotics (in the form of carbon sources, nitrogen sources, and other small molecules) and the introduction of bacteria can be used to shift the community composition, metabolic output, and immunological impact of the gut environments. This allows prebiotic, probiotic, and synbiotic (prebiotic and, probiotic) combinations to be rapidly evaluated to confirm or reject observations determined in simpler systems such as single strain microbial cultures. In subsequent embodiments, machine learning will be used to further reduce the space that must be explored experimentally.

### Application of Simulated Gut Environments to Isolated FMTs and Strains

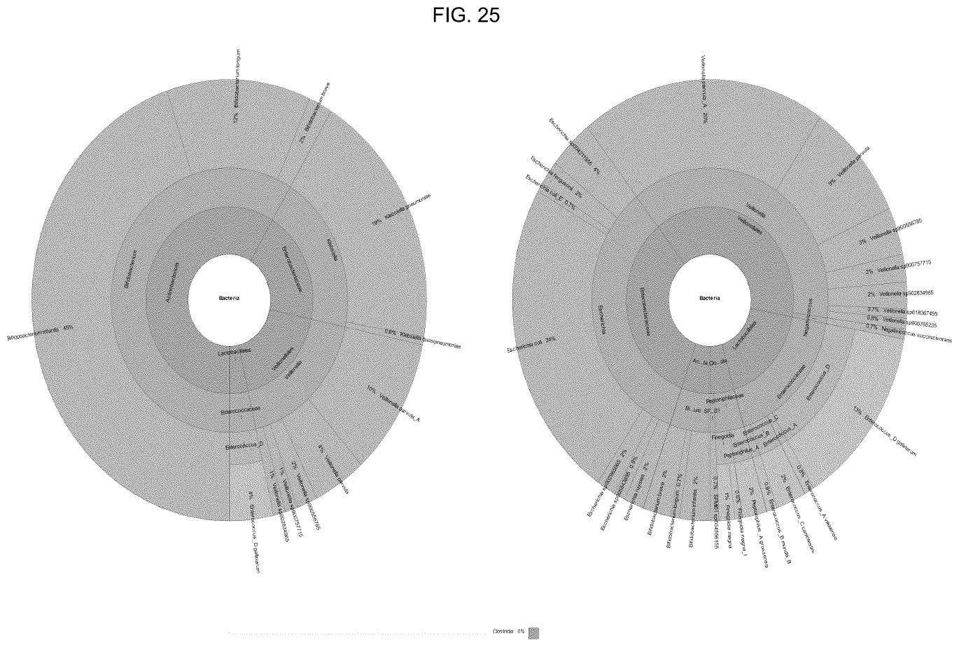

The ability of a carbon source to shift the composition of a simulated gut environment was first validated by evaluating outgrowth of a C1 sample in the presence of human milk oligosaccharides or infant formula (FIG. 25). In the presence of human milk oligosaccharides, the human gut environment maintained a C1 community structure, but in the presence of infant formula, the community structure shifted to that of a C3 community, demonstrating the significant impact of diet on the gut microbiome and the simulated gut environment.

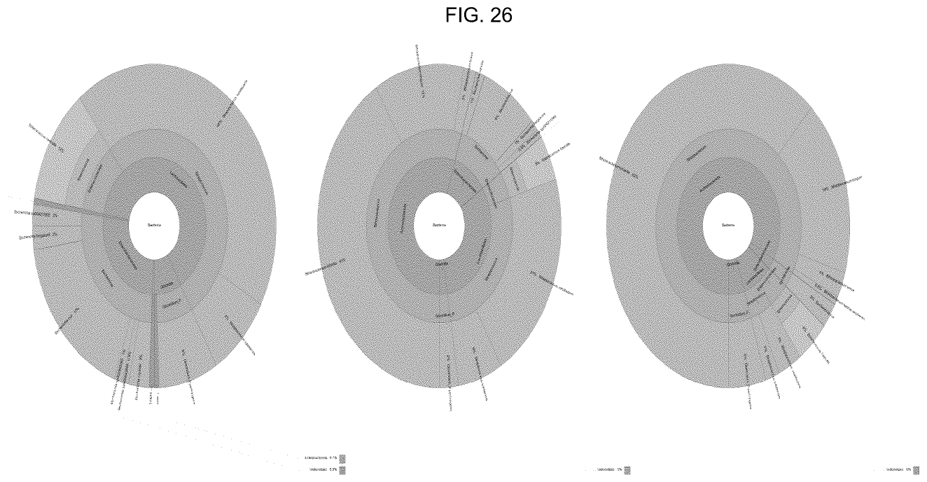

The ability of a bacterial addition (representing a probiotic application) to shift the composition of the gut environment was evaluated through the introduction of Bifidobacterium infantis isolate PB-STR-093. Bifidobacterium infantis was capable of shifting a C3 composition towards a C1 composition (FIG. 26), comprising over 50% of the sample after introduction. This was recapitulated in multiple FMTs, demonstrating reproducibility with the method. Beyond introducing prebiotics and probiotics alone, the combination of Bifidobacterium infantis with lacto-N-tetraose (a human milk oligosaccharide) was evaluated which led to further growth and engraftment in the sample (FIG. 26).

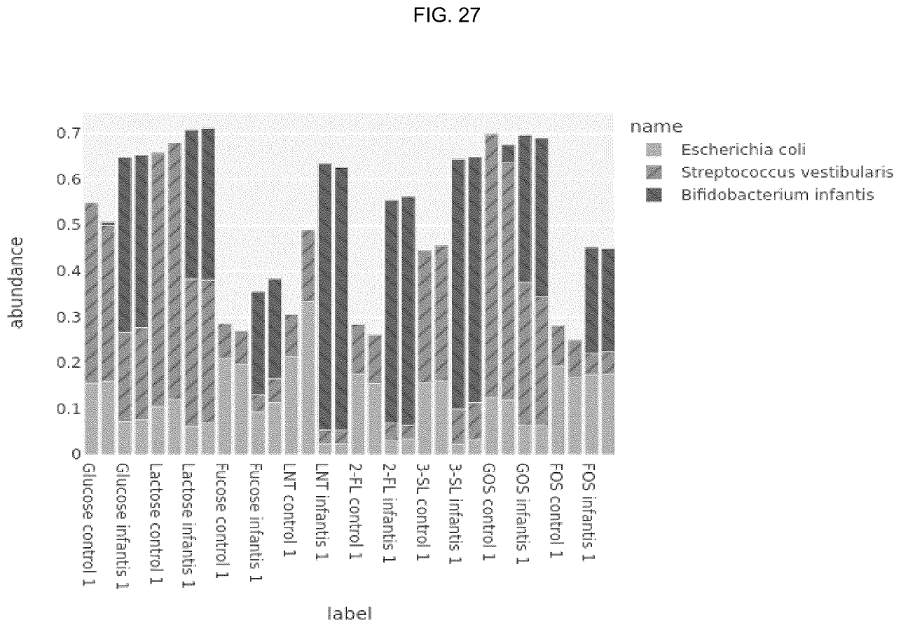

Notable among the compositional shifts was a significant reduction in potentially pathogenic bacterial strains (E. coli and S. vestibularis), particularly when growth was stimulated with lacto-N-tetraose relative to glucose (FIG. 27). In addition to compositional change, the ability to shift metabolism was also demonstrated.

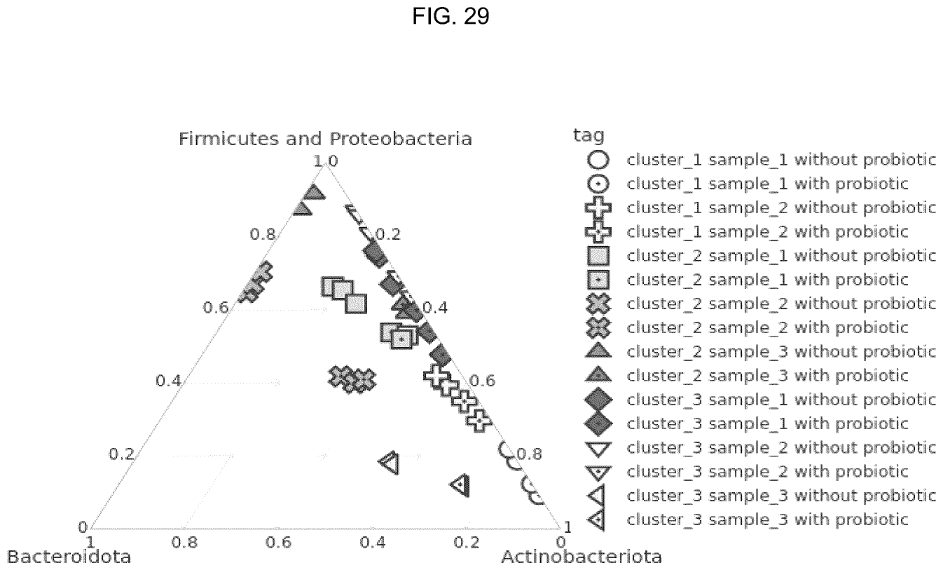

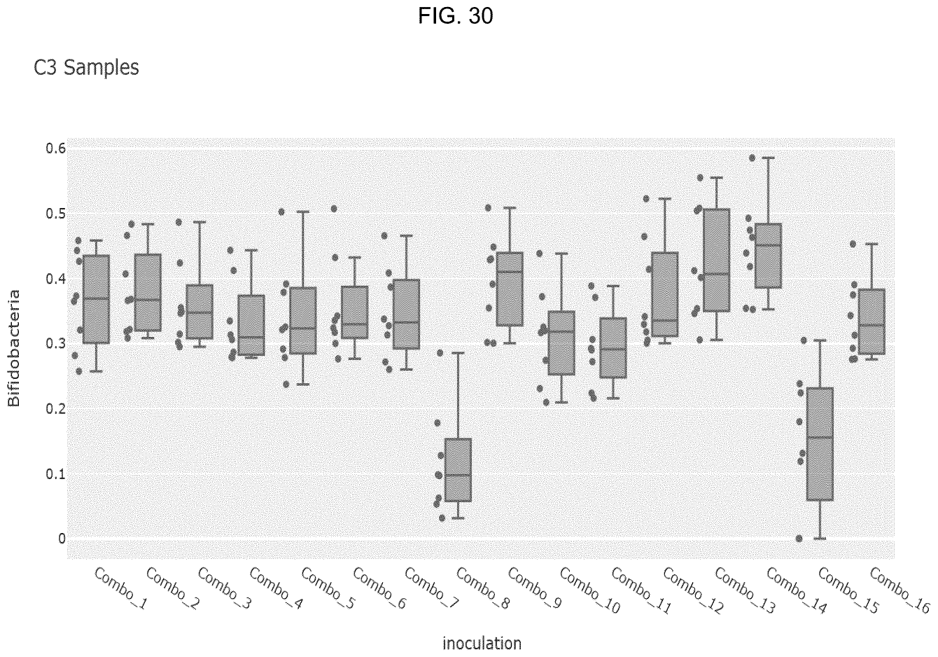

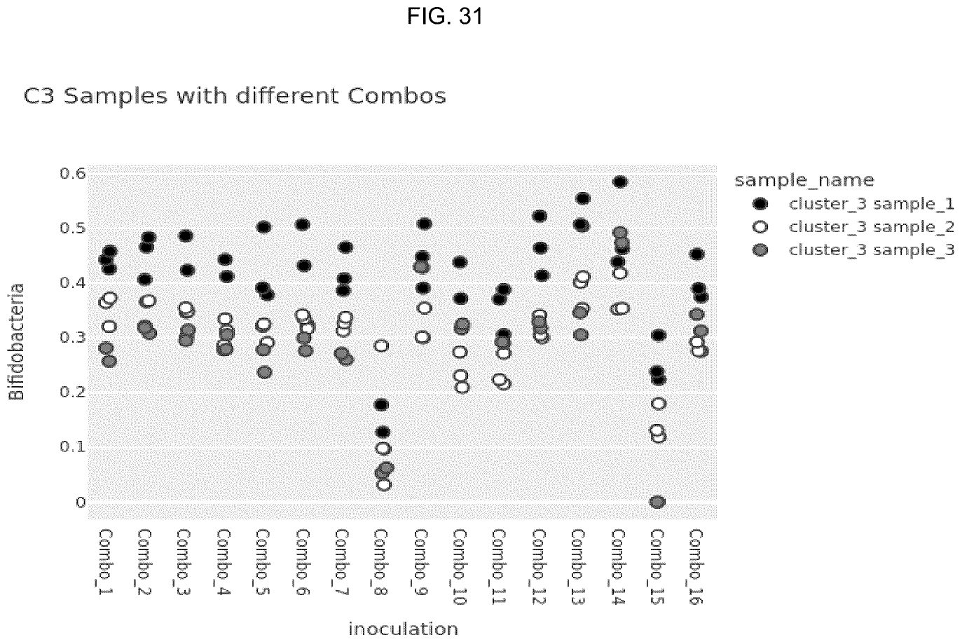

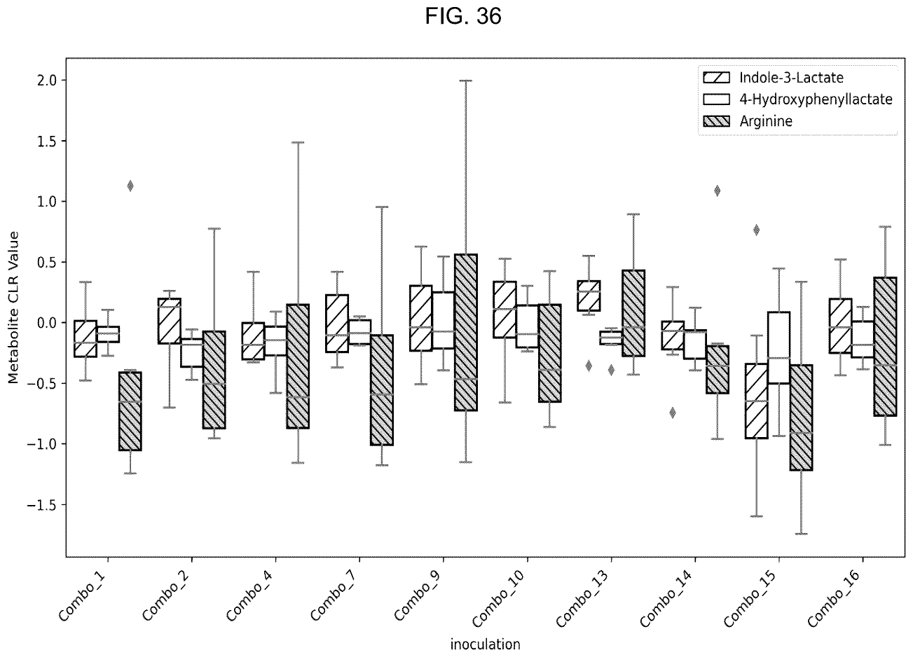

Based on the results of the MY BABY BIOME™ population wide analysis, combinations of Bifidobacterium were selected for their potential to improve infant health outcomes. Fifteen different combinations were evaluated covering Bifidobacterium infantis, Bifidobacterium longum, Bifidobacterium breve, and Bifidobacterium bifidum (shown in table 31, where the numerical value for each combination represents the ratio of the microbe in the combination). In all cases approximately the same number of bacteria were introduced into the system. In some combinations, multiple strains of the same species were evaluated and in other combinations relative levels of these strains were varied. These fifteen combinations were introduced into twenty unique gut environments (defined as a combination of an FMT background and a carbon source) covering a range of infant gut archetypes and evaluated for their ability to shift the composition of these backgrounds towards a C1 composition (FIG. 29, FIG. 30, FIG. 31). Compositions were also evaluated for their ability to reduce pathogenic species, as well as their metabolic output. A sample of metabolic output showing variation of 3 metabolites across different combinations is visualized in FIG. 36. The results of this analysis demonstrated that certain combinations excelled at remodeling the microbiome in the simulated gut environments, suggesting that these combinations were worth exploring further for their impact on the infant gut microbiome.

TABLE 31
Strain level combinations evaluated in simulated gut environments.

| Combi- | PB-STR- | PB-STR- | PB-STR- | PB-STR- | PB-STR- | PB-STR- | PB-STR- | PB-STR- | PB-STR- |
|---|---|---|---|---|---|---|---|---|---|
| nation | 093 | 083 | 119 | 101 | 103 | 220 | 207 | 215 | 335 |
| 1 | 2 | | 1 | | | 1 | | | |
| 2 | | 2 | 1 | | | 1 | | | |
| 3 | 1 | 1 | 1 | | | 1 | | | |
| 4 | 2 | 2 | 1 | | | 1 | | | |
| 5 | 3 | 1 | 1 | | | 1 | | | |
| 6 | 1 | 3 | 1 | | | 1 | | | |
| 7 | 2 | 2 | 1 | | | 1 | | | 1 |
| 8 | | | | | | | | | 1 |
| 9 | 2 | 2 | 1 | | | | 1 | | |
| 10 | 2 | 2 | | 1 | | | | 1 | |
| 11 | 1 | | | | | | | | |
| 12 | | 1 | | | | | | | |
| 13 | | | 1 | | | 1 | | | |
| 14 | | 1 | 1 | | | | | | |
| 16 | 2 | 2 | | | 1 | 1 | | | |

### Example 9: Laboratory-Scale Fermentation and Formulation of Isolated Anaerobic Microorganisms

In alternative embodiments, microbes used in compositions as provided herein, or used to practice methods as provided herein, comprise use of isolated anaerobic microorganisms, for example, anaerobic bacteria isolated from a fecal sample, for example, from a donor.

A laboratory-scale fermentation is performed using a Sartorius BIOSTAT A™ bioreactor with a 2-liter (L) vessel, using the growth media described in Example 1. While still in the anaerobic chamber, 1 L media is transferred to a sterile feed bottle, which has two ports with tubing leading blocked by pinch clamps and covered in foil to maintain sterility.

The fermentation vessel is sterilized by autoclaving, then flushed with a continuous purge of sterile nitrogen gas with oxygen catalytically removed. Two inlet ports are fitted with tubing leading to a connector blocked with a pinch clamp, and the sampling port fitted with tubing leading to a syringe. The vessel is also fitted with a dissolved oxygen probe, a pH probe, and a thermowell containing a temperature probe. Once anaerobic conditions are ensured, the media is removed from the anaerobic chamber and connected to one of the inlet ports. The other feed bottle port is connected to sterile nitrogen purge. The pinch clamp is removed, and media transferred into the fermentation vessel by peristaltic pump or just by the nitrogen pressure. Once the transfer is complete, both lines are sealed again by the pinch clamps, the feed bottle removed, and returned to the anaerobic chamber.

A 50 mL seed culture of one or more bacteria from the following genera (any one of which are used to practice compositions or methods as provided herein), Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), Acetatifactor (TaxID: 1427378), Adlercreutzia (TaxID: 447020), Agathobacter (TaxID: 1766253), Anaerotruncus (TaxID: 244127), Bariatricus (TaxID: 1924081), Butyrivibrio (TaxID: 830), Christensenellaceae (TaxID: 990719), Clostridiales (TaxID: 186802), Dialister (TaxID: 39948), Drancourtella (TaxID: 1903506), Eggerthella (TaxID: 84111), Eisenbergiella (TaxID: 1432051), Enterocloster (TaxID: 2719313), Enterococcus (TaxID: 1350), Intestinibacter (TaxID: 1505657), Lachnospira (TaxID: 28050), Lachnospiraceae (TaxID: 186803), Mediterraneibacter (TaxID: 2316020), Negativibacillus (TaxID: 1980693), Oscillibacter (TaxID: 459786), Phocaeicola (TaxID: 909656), Pseudobutyrivibrio (TaxID: 46205), Pseudoflavonifractor (TaxID: 1017280), Ruminococcaceae (TaxID: 541000), Sellimonas (TaxID: 1769710), Solobacterium (TaxID: 123375), Terrisporobacter (TaxID: 1505652), Tidjanibacter (TaxID: 1929083), Veillonella (TaxID: 29465), Lacticaseibacillus (TaxID: 2759736), or Limosilactobacillus (TaxID: 2742598) are grown to mid-exponential phase in a sealed culture bottle using the same media composition as above, and are transferred into the feed bottle in the anaerobic chamber. Repeating the above transfer procedure, this time with the culture, the fermenter is inoculated.

5 M ammonium hydroxide is prepared in another feed bottle. One port is connected to sterile nitrogen, and the bottle is purged for 5 minutes to remove all oxygen. The outlet tubing is then blocked by a pinch clamp and attached to the other inlet port in the fermentation vessel. This tubing is then threaded into a peristaltic pump head, and the pinch clamp removed. Using the software built into the Biostat A™ unit, this pump is controlled to maintain pH at 7.0.

During growth of the culture, temperature is maintained at 37° C. using a temperature controller and heating blanket on the vessel. Nitrogen purge is set at 0.5 L/min to maintain anaerobic conditions and positive pressure in the vessel, and agitation is set at 500 rpm to keep the culture well mixed. Periodic samples are taken using the syringe attached to the sample port. For each sample, optical density is measured at 600 nm wavelength using a spectrophotometer.

### Example 10: Stability Testing of Microbes

In alternative embodiments, microbes used in compositions as provided herein, or used to practice methods as provided herein, comprise or can be derived from any one of family or genus (or class): Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), Acetatifactor (TaxID: 1427378), Adlercreutzia (TaxID: 447020), Agathobacter (TaxID: 1766253), Anaerotruncus (TaxID: 244127), Bariatricus (TaxID: 1924081), Butyrivibrio (TaxID: 830), Christensenellaceae (TaxID: 990719), Clostridiales (TaxID: 186802), Dialister (TaxID: 39948), Drancourtella (TaxID: 1903506), Eggerthella (TaxID: 84111), Eisenbergiella (TaxID: 1432051), Enterocloster (TaxID: 2719313), Enterococcus (TaxID: 1350), Intestinibacter (TaxID: 1505657), Lachnospira (TaxID: 28050), Lachnospiraceae (TaxID: 186803), Mediterraneibacter (TaxID: 2316020), Negativibacillus (TaxID: 1980693), Oscillibacter (TaxID: 459786), Phocaeicola (TaxID: 909656), Pseudobutyrivibrio (TaxID: 46205), Pseudoflavonifractor (TaxID: 1017280), Ruminococcaceae (TaxID: 541000), Sellimonas (TaxID: 1769710), Solobacterium (TaxID: 123375), Terrisporobacter (TaxID: 1505652), Tidjanibacter (TaxID: 1929083), Veillonella (TaxID: 29465), Lacticaseibacillus (TaxID: 2759736), Limosilactobacillus (TaxID: 2742598), or a combination thereof.

In alternative embodiments, any microbe used in a composition as provided herein, or used to practice methods as provided herein, for example, including a microbe as listed above, can be stored in a sealed container, for example, at 25° C. or 4° C. and the container can be placed in an atmosphere having 30%, 40%, 50%, 60%, 70%, 75%, 80%, 90% or 95% relative humidity, or between about 20% and 99% relative humidity. In alternative embodiments, after 1 month, 2 months, 3 months, 6 months, 1 year, 1.5 years, 2 years, 2.5 years or 3 years, at least 50%, 60%, 70%, 80% or 90% of the bacterial strain shall remain as measured in colony forming units determined by standard protocols.

### Example 11: Production of Live Biotherapeutics for Evaluation of Immunological Impact In Vivo

In alternative embodiments, microbes as provided herein (including bacteria from all the genera listed herein), and including the combinations of microbes as provided herein, for example, the exemplary combinations listed in Example 4 or Example 5, comprise anaerobic bacteria, including anaerobic bacteria isolated from a fecal sample, cultured anaerobic bacteria, or a combination thereof.

### Individual Culture of Anaerobic Microbes for Mouse Studies

Anaerobic microbes of interest are cultured in multiples of 1-liter volumes in anaerobic media bottles as follows. Microbes in cryostorage are plated and struck on appropriate anaerobic solid medium and then cultured at 37° C. to obtain isolated colonies. For each microbe, a single colony is inoculated into a Hungate tube containing 10 ml appropriate anaerobic growth medium and allowed to grow at 37° C. until turbid to create a starter culture. For each microbe of interest, multiple 0.9-liter volumes of appropriate liquid anaerobic medium in 1 L anaerobic bottles (as described in Example 1) are inoculated with 2 ml starter culture each using a needle and syringe. The number of 1-liter cultures for each microbe is dependent on the necessary final amount of live cell mass for formulation into live biotherapeutics for mouse studies. Inoculated bottles are placed upright on a platform shaker at 115 rpm at 37° C. for 48 hours or until growth turbidity is evident. Growth density is monitored by taking 1 ml samples during the course of the cultures for optical density measurements at 600 nm. Optical densities of 1.0 to 4.0 can be obtained after 48 hours depending on the microbe cultured. Prior to large scale culture, cell densities are determined empirically for each microbe by dilution plating and colony counting to determine the colony forming units (CFU) per ml at an optical density of 1.0.

Large scale cultures are grown to attain a final live density of 10⁸ to 10⁹ CFU/ml, and then the culture bottles are brought into the anaerobic chamber for harvesting of live cell mass. Once in the chamber, the aluminum collars and butyl rubber bungs are removed, and the 1-liter contents of each culture bottle are poured into two 500 ml centrifuge bottles with rubber gasketed screw caps. After decanting the growth medium, the caps of the centrifuge bottles are tightened for an airtight seal, brought out of the anaerobic chamber, then centrifuged for 20 minutes at 6000 g at 4° C. Centrifuged bottles are then brought into the anaerobic chamber, uncapped, and then the supernatants are poured off and discarded. The remaining cell pellets are then combined with 250 ml ice cold Vehicle Buffer (Phosphate Buffered Saline plus 1 g/L L-cysteine plus 15% glycerol, filter sterilized and made anoxic by bubbling with filtered nitrogen). The cell pellets are carefully resuspended in the Vehicle Buffer on ice; the resuspended volumes of two pellets are combined into one 500 ml bottle, recapped for an air-tight seal, removed from the anaerobic chamber, then centrifuged for 20 minutes at 6000 g at 4° C. After decanting supernatants in the anaerobic chamber, resulting cell pellets are then carefully resuspended once more with 250 ml ice cold Vehicle Buffer in the anaerobic chamber, removed from the anaerobic chamber, then centrifuged for 20 minutes at 6000 g at 4° C. After removal of supernatant in the anaerobic chamber, each pellet is resuspended in 100 ml ice cold Vehicle Buffer to establish a ten-fold concentration of the original culture cell density.

Within the anaerobic chamber, final resuspended cell pellet volumes for an anaerobic microbe of interest are combined and thoroughly mixed in a sterile bottle by gentle stirring on a stir plate on ice. The volume is then dispensed into 25 ml aliquots in 50 ml conical tubes using a serological pipette, then a stream of sterile filtered gaseous argon is introduced to each tube to displace the headspace and to serve as an oxygen barrier. Each tube is then tightly capped, and the seal is wrapped with several layers of parafilm. The tubes are then racked upright, removed from the anaerobic chamber, and then allowed to slowly freeze at −80° C. A smaller 5 ml aliquot is also made for each preparation and stored as described above. After 18 hours, the 5 ml aliquots for each microbial strain of interest are removed and allowed to thaw standing in ice water within the anaerobic chamber. The thawed volumes are gently mixed by inversion several times, then subjected to dilution plating on appropriate solid anaerobic medium to determine the live cell density in CFU/ml after freezer storage.

### Live Biotherapeutic Assembly for Mouse Studies

Live biotherapeutic compositions of anaerobic microbes of interest, including the combinations of microbes as provided herein, for example, the exemplary combinations described in Example 4 or Example 5, are assembled in volumes that are pertinent for projected mouse studies. Enough aliquots for each microbe of interest are removed from storage at −80° C. and gently thawed in ice water in the anaerobic chamber. The thawed multiple aliquots are combined in a sterile bottle, gently remixed and then placed on ice. The amount of volume of each microbe to add to a mix is adjusted so that the determined live cell densities for each microbe are equivalent, and final total cell densities can be adjusted by further addition of ice-cold vehicle buffer. Once all requisite volumes for each microbe are added together in a larger sterile bottle, the volume is gently mixed by stirring on a stir plate on ice.

Live biotherapeutic volumes are then re-aliquoted in individual volumes that each comprise a projected daily dose of live microbes in anticipated mouse studies. Determined volumes are each dispensed in 15 ml conical tubes up to 10 ml per aliquot. The volume in each tube is overlaid with a stream of sterile filtered argon to displace oxygen, followed by capping. Live biotherapeutic aliquot tubes are racked upright and allowed to slowly freeze at −80° C. After 48 hours, one aliquot for each microbe mix preparation is thawed and dilution plated to validate the final total CFU/ml, optimally at greater than 1.0×10⁹ CFU/ml.

### Scaled Manufacturing of Strains

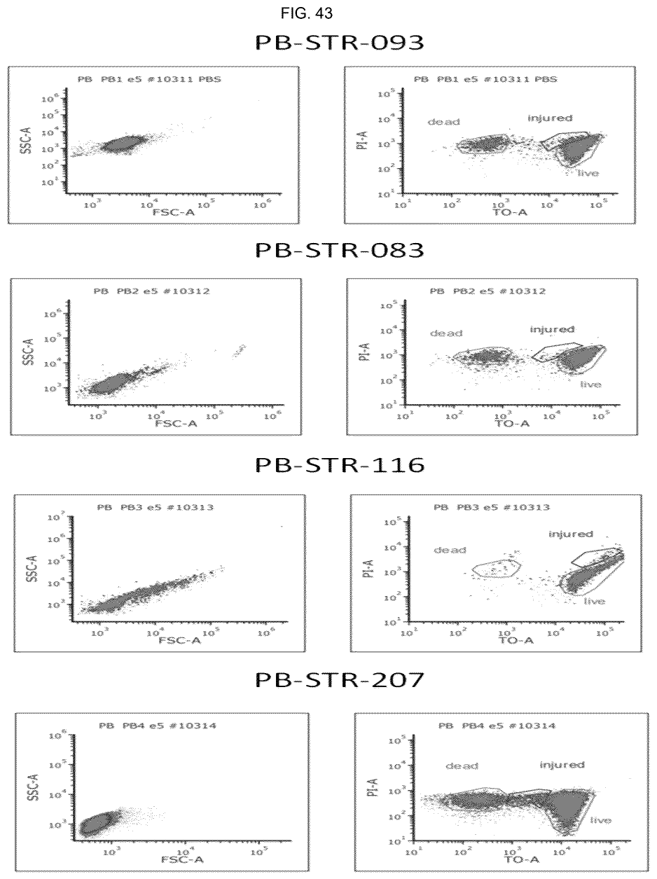

Strains PB-STR-093, PB-STR-083, PB-STR-119, and PB-STR-207 were produced at a larger scale to generate enough material for additional evaluation, first at one liter and then at seven liters. All strains grew successfully in a minimal media and demonstrated retention of viability after freeze drying. Examples of excipients used in freeze drying include but are not limited to acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium stearate, calcium disodium EDTA, copolyvidone, calcium hydrogen phosphate dihydrate, cetylpyridine chloride, cysteine HCL, crossprovidone, calcium phosphate di or tri basic, dibasic calcium phosphate, disodium hydrogen phosphate, dimethicone, erythrosine sodium, ethyl cellulose, gelatin, glyceryl monooleate, glycerin, glycine, glyceryl monostearate, glyceryl behenate, hydroxy propyl cellulose, hydroxyl propyl methyl cellulose, hypromellose, HPMC phthalate, inulin, iron oxides or ferric oxide, iron oxide yellow, iron oxide red or ferric oxide, lactose hydrous or anhydrous or monohydrate or spray dried, magnesium stearate, maltodextrin, microcrystalline cellulose, mannitol, methyl cellulose, magnesium carbonate, mineral oil, methacrylic acid copolymer, magnesium oxide, methyl paraben, providone or PVP, PEG, polysorbate 80, propylene glycol, polyethylene oxide, propylene paraben, polaxamer 407 or 188, potassium bicarbonate, potassium sorbate, potato starch, phosphoric acid, polyoxy 140 stearate, sodium starch glycolate, starch pregelatinized, sodium carmellose, sodium lauryl sulfate, starch, silicon dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dihydrate, sodium starch, sodium carboxy methyl cellulose, succinic acid, sodium propionate, titanium dioxide, talc, triacetin, and triethyl citrate. Following freeze drying, isolated cells were evaluated for purity and quality, and viability was evaluated both as colony forming units (CFU) and active fluorescence units (AFU) (FIG. 43). CFU and AFU closely matched for each strain, suggesting both as a reasonable measure of viability.

### Example 12: Demonstration of Immunological Impact In Vivo

### Microorganisms in Mouse Study

The sets of microbes to be administered are chosen from those described in Example 4 or Example 5. Each microbe is isolated from healthy donors, as described in Example 3. After assembly of the consortia as described in Example 11, PBS-C-G is added to each live biotherapeutic to reduce the total cell density of each live biotherapeutic to the desired dosage level, which can be between 1×10⁷/0.2 ml and 1×10¹²/0.2 ml. Live biotherapeutics are aliquoted into 15 ml conical tubes in single use volumes and stored at −20° C. until required.

### Animals

BALB/c mice are obtained from SHANGHAI LINGCHANG BIOTECHNOLOGY CO., LTD™ (Shanghai, China), JACKSON LABORATORY™ or another mouse facility. 6-8-week-old female mice are used. To prepare mice for fecal microbiota transplant mice are treated daily with 200 μL of antibiotic solution via oral gavage for a duration of 1-2 weeks. The antibiotic solution consists of ampicillin (1 mg/mL) (Alfa Aesar J6380706), gentamicin (1 mg/mL) (Acros Organics AC455310050), metronidazole (1 mg/mL) (Acros Organics AC210440050), neomycin (1 mg/mL) (Alfa Aesar AAJ6149922), and vancomycin (0.5 mg/mL) (Alfa Aesar J6279006) via oral gavage. Animals are given a 24 hour rest period between antibiotic pre-treatment and the treatment phase to allow for antibiotics to go through the system, and the cage is changed prior upon inoculation with the fecal microbiota transplant.

### Fecal Microbiota Transplantation (FMT)

Fecal Microbiota Transplantation (FMT) of a human gut microbiome into antibiotic treated mice is a method for standardizing microbiome composition. To evaluate the impact of probiotic treatment on the immune system in vivo, mice are divided into two groups and either treated with a microbiome representing a robust infant microbial composition (C1 from Example 3) or poor infant microbial composition (C3 as described in Example 3). Not only does this standardize the mice microbiomes, but also conditions them towards two diverse immunological states (as shown in Example 5). Following antibiotic pre-treatment, colonization is performed by oral gavage with 200 μl of suspension obtained by homogenizing the fecal samples in PBS. Mouse fecal samples are collected 1-2 times during this period, so that the efficacy of the FMT can be evaluated. Following FMT, a rest period of 5-7 days is allowed to pass prior to probiotic treatment.

### Probiotic Treatment

Mice with each microbiome (C1 and C3) are randomized and divided into two groups, one control group and one treatment group. Mice are marked by ear tagging. Mice in the treatment group are treated with 200 μl oral gavage of microbe mix (between 10⁷ and 10¹² colony forming units, CFU, per dose) and mice in the control group are treated with 200 μl of vehicle control. Treatment continues for three weeks or longer. Doses are administered at a frequency of at least twice per week and up to daily. Stool is collected upon inoculation and at least twice per week until the end of the study.

### Peripheral Blood Extraction and Processing

Whole blood is taken via cardiac puncture at the end of the experiment, or via tail bleed during the experiment, and collected into an EDTA tube. Plasma is isolated from an aliquot of the whole blood by centrifugation at 1500×g for 10 minutes, taking the supernatant. A second centrifugation is performed to remove any residual blood cells. Peripheral blood mononuclear cells (PBMCs) are isolated from blood using a standard kit and stored in liquid nitrogen at 1×10⁶ cells/mL until use. Prior to storage, PBMC's may be processed using flow sorting or antibody spin separation kit to select for a certain purified lymphocyte subpopulation, such as T cells.

### GI Tract Removal and Analysis

After mice are euthanized at the termination of the study, the intact digestive tract of each mouse from stomach to rectum are removed and kept in a 5 ml Eppendorf tube on ice prior to dissection. Forceps are sterilized by soaking in 100% ethanol and then used to remove the intestine length and stretch it on a work surface covered with cellophane. With the use of ethanol-sterilized dissection scissors, 3 cm lengths of the jejunum nearest to the stomach and the ilium nearest to the cecum/large intestine are excised and then each placed with forceps in a 1.5 ml Eppendorf tube and placed on ice. A 2 cm segment of the cecum/ascending colon is then excised, as are 2 cm segments of the transcending colon and the descending colon, and all are placed in 1.5 ml Eppendorf tubes on ice. Dissection instruments are sterilized by dipping in 100% ethanol between each intestine fragment removal. To each tube containing dissected intestinal segments is added 0.5 ml ice cold PBS buffer. A plastic pestle is used to press and massage the intestinal segment in each tube to expel ruminal matter, which is then removed by pipette and placed in a fresh Eppendorf tube. Tubes containing expelled ruminal matter from each intestinal segment are immediately placed on dry ice and then stored for later analyses at −80° C. Remaining intestinal tissues are then rinsed twice by adding and then removing 0.5 ml ice cold PBS. Rinsed intestinal fragment tissues are then frozen on dry ice and then stored at −80° C. for later analysis.

### Analyses of Dendritic Cell Subsets

Cell suspensions from mouse spleen and lymph nodes are prepared by digestion with collagenase and Dnase for 60 min and subsequently strained through a 70 mm mesh. Colonic and small intestinal lymphocytes are isolated as previously described (Viaud, S. et al. Science 80 (342): 971-976 (2013). In brief, cecum, colon and small intestine are digested in PBS containing 5 mM EDTA and 2 mM DTT shaking at 37° C. A plastic pestle is used to press and massage the intestinal segment in each tube to expel ruminal matter, which is then removed by pipette and placed in a fresh Eppendorf tube. Tubes containing expelled ruminal matter from each intestinal segment are immediately placed on dry ice and then stored for later analyses at −80° C. Remaining intestinal tissues are then rinsed twice by adding and then removing 0.5 ml ice cold PBS. Rinsed intestinal fragment tissues are then frozen on dry ice in RNALATER™ (Thermo Fisher Scientific) and then stored at −80° C. for later analysis. After initial digestion colonic and small intestinal tissue pieces are digested in collagenase/Dnase containing RPMI medium for 30 min. Tissue pieces are further strained through a 70 mm mesh. For flow cytometry analyses, cell suspensions are stained with antibodies against the following surface markers: CD11c (N418), CD11b (M1/70), Ly6c (HK1.4), MHC class II (M5/114.15.2), CD24 (M1/69), CD64 (X54-5/7.1), CD317 (ebio927), CD45 (30-F11), F4/80 (C1: A3-1), CD8a (53-6.7). DAPI is used for dead cell exclusion. Antibodies are purchased from EBIOSCIENCES, BD BIOSCIENCES™ or BIOLEGEND™ respectively. Cell populations are gated as follows: small intestine (migratory fraction): CD103+DC (CD45+CD11c+MHC-II+CD103+CD24+), CD11b+CD103+ (CD45+CD11c+MHC-II+CD103+CD11b+CD24+), CD11b+ (CD45+CD11c+MHC-II+CD11b+CD24+), inflammatory DC (CD45+CD11c+MHC-II+CD11b+CD64+Ly6c+), large intestine: CD103+DC (CD45+CD11c+MHC-II+CD103+CD24+), CD11b+ (CD45+CD11c+MHC-II+CD11b+CD24+), inflammatory DC (CD45+CD11c+MHC-II+CD11b+CD64+Ly6c+).

### Whole Genome Sequencing

Fecal gDNA is extracted for whole genome sequencing (WGS). Experimental methods for DNA extraction and library preparation are performed using protocols modeled after the Human Microbiome Project (Lloyd-Price et al. (2017) Nature 550 (7674): 61-66) and validated with samples from healthy volunteers. Sequencing is performed by an outside service provider, using a HISEQ-X® (Illumina) with 2×150 bp paired-end reads, providing approximately 4 million reads per sample. Analysis software such as Centrifuge (Kim, D., et al., Centrifuge: rapid and sensitive classification of metagenomic sequences. Genome Res, 2016. 26 (12): p. 1721-1729) are used to align sequence reads to reference genomes and obtain species and strain-level identification.

### Metabolomics

Metabolites are extracted from fecal material or blood plasma, using methanol under vigorous shaking for 2 min (GENOGRINDER 2000™ (Glen Mills)) to precipitate protein and dissociate small molecules bound to protein or trapped in the precipitated protein matrix, followed by centrifugation to recover chemically diverse metabolites. The resulting extract is evaluated through targeted metabolomics as described in Example 4 or through untargeted metabolomics. For targeted metabolomics, samples are placed on a TURBOVAP® (Zymark) to remove the organic solvent prior to evaluation. Compounds are identified by comparison to known standards with associated calibration curves. Absolute quantification is achieved through the use of isotopically labeled internal standards. For untargeted metabolomics, samples are placed on a TURBOVAP® (Zymark) to remove the organic solvent, before being evaluated in one of the following ways: reverse phase (RP)/UPLC-MS/MS using positive ion mode electrospray ionization (ESI), RP/UPLC-MS/MS using negative ion mode ESI, HILIC/UPLC-MS/MS using negative ion mode ESI, or HILIC/UPLC-MS/MS using positive ion mode ESI. Compounds are identified by comparison to library entries of purified standards that contain the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library+/−10 ppm, and the MS/MS forward and reverse scores. MS/MS scores are based on a comparison of the ions present in the experimental spectrum to ions present in the library entry spectrum. While there may be similarities between these molecules based on one of these factors, the use of all three data points can be utilized to distinguish and differentiate biochemicals. Peaks are quantified as area-under-the-curve detector ion counts.

### Immunophenotyping Assays

Immune profiling of whole blood is utilized to assess T cell activation in response to microbial treatment. In some experiments, immune phenotyping is also performed on tissue obtained from the GI tract. For flow cytometry analysis, 1 mL of RBC Lysis Buffer is added to 0.1 mL of whole blood or homogenized tissue and allowed to incubate at room temperature for 10 minutes. Lysis is quenched by adding 10 mL of cold DPBS. Samples are centrifuged at 1500 rpm for 5 minutes at 4° C. The pellet is aspirated and resuspended in another 10 mL of cold DPBS. Samples are centrifuged at 1500 rpm for 5 minutes at 4° C. Samples are resuspended in 500 μL of FACS buffer and transferred to a 96-well plate. Samples are stained with Fixable Viability ef780™ (eBioscience), CD45-Pecy7 (BioLegend), CD3-BV605™ (BioLegend), CD8-AF700™ (BioLegend), and CD4-AF488™ (BioLegend). Stained samples are run on a BD LSRFortessa™ flow cytometer and analyses are performed with FLOWJO™ (Tree Star).

Alternatively, CyTOF® is applied to characterize the immune profile of the PBMCs. This work is conducted by the Bioanalytical and Single-Cell Facility at the University of Texas, San Antonio, and entails a comprehensive panel of 29 different immune markers, allowing for deep interrogation of cellular phenotype and function. To complement these results, RNA sequencing is applied to the entire population of the PBMCs, sorted populations, and also to single cells. Single cell RNAseq is applied using the method developed by 10× GENOMICS™. Finally, cytokine levels are determined using an assay such as the HUMAN CYTOKINE 30-PLEX LUMINEX™ assay.

### Example 13: Observational and Interventional Clinical Studies on Colorectal Cancer Risk

Stool and blood samples are collected from cancer patients and healthy individuals classified as high or low risk for colorectal cancer (CRC) based on family history, prior CRC, or prior colonoscopy findings. Specifically, high risk subjects are those meeting one of the following criteria:
1. Family history of CRC (one or more first-degree relatives) OR
2. One or more of the following findings during prior colonoscopy:
   Adenoma greater than (>) 10 mm in size
   Adenoma with tubulovillous/villous histology
   Adenoma with high-grade dysplasia
   CRC
   3 or more non-advanced adenomas, regardless of size, histology, or dysplasia found during a single screening or surveillance visit

Subjects who meet the entry criteria provide a baseline stool sample and up to 4 samples over a 2-year period. Stool sample collection is performed as described in Example 2. Blood samples are also collected from some participants. Electronic health records are obtained, and patient lifestyle questionnaires are administered at various times for an additional 3-6 years. In some cases, additional samples will be collected in conjunction with significant clinical events, such as another standard of care colonoscopy or recurrence of CRC with or without surgical intervention.

Some of the high risk subjects are given interventions, to determine the effect of nutritional counseling or probiotic supplements on microbiome composition and function. These subjects are assigned to cohorts as follows:

Cohort 1 is the control group with no intervention.

Cohort 2 participants will have 6 tele-health visits with a nutritionist to provide dietary guidance. The first visit is within 3 weeks after providing the stool sample, with one visit every month subsequently. The nutritionist works with the participants to design a diet plan optimized to improve microbiome health and meet their overall health objectives.

Cohort 3 participants are given a daily probiotic supplement during the duration of the study, beginning within 3 weeks of the initial stool sample.

Cohort 4 participants will receive both the daily probiotic and dietary consultation.

Stool samples are collected after 3 months and 6 months of intervention. Metagenomics, metabolomics, and cytokine analysis are performed as described in Example 3.

### Example 14: Exemplary Methods for Collection and Analysis of Stool from Mothers and Infants

In a maternal health focused clinical study, stool samples are collected from expectant mothers in their third trimester of pregnancy. Subsequently, samples are collected from infants between 4 and 10 weeks after delivery. Mothers provide demographic, diet, and lifestyle information, as well as document birth mode and feeding method. Antibiotic or probiotic use by either the mother or infant is also captured. Periodic surveys are filled out for up to 7 years, capturing health information as the baby grows. Subsequent stool samples are also collected from some babies. Some mothers provide a breast milk sample. Samples are processed as described in Example 2. Metagenomics, metabolomics, and proteomics are performed as described in Example 3, and multigenerational transmission of gut microbes is assessed.

### Example 15: Exemplary Methods of Treating an Infant with a Live Biotherapeutic (or Probiotic) for the Treatment and Prevention of Dysbiosis that can Lead to Disease

This example describes administration of a live biotherapeutic (or probiotic) as provided herein, which in alternative embodiments comprises one bacteria and a probiotic (or a synbiotic, for example, as set forth in Table 8 or Table 32, below), or alternatively comprises a combination or mix (or consortium) of bacteria as provided herein, for example, as set forth in Table 2, Example 4, or Table 30, Example 5, and/or administration of prebiotic as provided herein, including a combination of prebiotics as provided herein, for example, as set forth in Table 3, Example 4, to an infant or newborn in need thereof.

An infant gut is dysbiotic or at risk of becoming dysbiotic. Infant dysbiosis can be defined as but is not limited to infants with bacterial compositions described in Example 3, infants with primary HMO consumers that are not Bifidobacterium, infants whose gut metabolic and immunological state differs substantially from that of a Bifidobacterium dominated gut, infants whose dominant Bifidobacterium are rarely found in the dataset described in Example 3, and infants with a microbiome composition that has been associated with diseases later in life such as asthma, allergies, obesity, and diabetes. The infant is administered a live biotherapeutic composition, i.e., a formulation consisting of some combination of microbes as outlined in Example 4 or Example 5 either alone or in combination with prebiotics or supplements as outlined in Example 4 to address the dysbiosis.

In alternative embodiments, each or one of the microbes used in the bacterial combination is (at least initially) isolated from a healthy donor or donors, as described in Example 5, or is a genetically modified derivative as described in Example 21, or is a cultured derivative either.

In alternative embodiments, the patient is administered the composition, formulation or pharmaceutical formulation (for example, probiotic) at a dose of between about 10⁵ to 10¹⁵ bacteria, once, twice, three times, or more often per day. Dosing can occur through a lyophilized form, i.e. a lyophilized powder that is given orally to the infant either directly, through a dropper, or through a bottle.

In another embodiment, the infant may be dosed with the composition, formulation or pharmaceutical formulation (for example, probiotic) before, during, and/or immediately after feeding.

In another embodiment, dosing of the composition, formulation or pharmaceutical formulation (for example, probiotic) is continued for 1 month, 6 months, 1 year, or more.

In alternative embodiments, the composition of the infant's gut microbiome and the metabolic and immunological state of the infant gut are used as a measure of successful treatment.

A twenty infant, decentralized, placebo-controlled study was used to evaluate a probiotic combination as outlined in Example 4 or Example 5. Stool samples were collected at multiple timepoints (as outlined in Example 2) and evaluated for the stability of the Bifidobacterium population, the impact of Bifidobacterium on gut metabolism, and the reduction in pathogen or undesired microbe content.

### Example 16: Method of Treating an Infant Following Disruption of the Microbiome due to Cesarean-Section Birth or Antibiotic Use

This example describes administration of a live biotherapeutic (or probiotic) as provided herein, which in alternative embodiments comprises one bacteria and a probiotic (or a synbiotic, for example, as set forth in Table 8 or Table 32, below), or alternatively comprises a combination or mix (or consortium) of bacteria as provided herein, for example, as set forth in Table 2, Example 4, or Table 30, Example 5, and/or administration of prebiotic as provided herein, including a combination of prebiotics as provided herein, for example, as set forth in Table 3, Example 4, to an infant or newborn in need thereof.

An infant has undergone an event such as Cesarean-section birth or antibiotic use that can lead to disruption of the microbiome. Disruption of the microbiome in an infant can be defined as but not limited to, a reduction in Bifidobacterium, an alteration in the metabolic and immunological state of the gut, an increase in pathogens or undesired microbes, or alteration of the microbiome composition to reflect one that has been associated with diseases later in life such as asthma, allergies, obesity, and diabetes. The infant is administered a live biotherapeutic composition, i.e., a formulation consisting of some combination of microbes as outlined in Example 4 or Example 5 either alone or in combination with prebiotics or supplements as outlined in Example 4 to address the disruption.

In alternative embodiments, each or one of the microbes used in the bacterial combination is (at least initially) isolated from a healthy donor or donors, as described in Example 5, or is a genetically modified derivative as described in Example 21, or is a cultured derivative either.

In alternative embodiments, the patient is administered the composition, formulation or pharmaceutical formulation (for example, probiotic) at a dose of between about 10⁵ to 10¹⁵ bacteria, once, twice, three times, or more often per day. Dosing can occur through a lyophilized form, i.e. a lyophilized powder that is given orally to the infant either directly, through a dropper, or through a bottle.

In another embodiment, the infant may be dosed with the composition, formulation or pharmaceutical formulation (for example, probiotic) before, during, and/or immediately after feeding.

In another embodiment, dosing of the composition, formulation or pharmaceutical formulation (for example, probiotic) is continued for 1 month, 6 months, 1 year, or more.

In alternative embodiments, the composition of the infant's gut microbiome and the metabolic and immunological state of the infant gut are used as a measure of successful treatment.

A twenty infant, decentralized, placebo-controlled study was used to evaluate a probiotic combination as outlined in Example 4 or Example 5. Stool samples were collected at multiple timepoints (as outlined in Example 2) and evaluated for the stability of the Bifidobacterium population, the impact of Bifidobacterium on gut metabolism, and the reduction in pathogen or undesired microbe content.

### Example 16: Method of Treating an Infant Suffering from an Immune-Related Disorder

This example describes administration of a live biotherapeutic (or probiotic) as provided herein, which in alternative embodiments comprises one bacteria and a probiotic (or a synbiotic, for example, as set forth in Table 8 or Table 32, below), or alternatively comprises a combination or mix (or consortium) of bacteria as provided herein, for example, as set forth in Table 2, Example 4, or Table 30, Example 5, and/or administration of prebiotic as provided herein, including a combination of prebiotics as provided herein, for example, as set forth in Table 3, Example 4, to an infant or newborn in need thereof.

An infant has begun displaying symptoms of an immune-related disorder, such as development of allergies, dermatitis, asthma, or obesity. The infant is administered a live biotherapeutic composition, i.e., a formulation consisting of some combination of microbes as outlined in Example 4 or Example 5 either alone or in combination with prebiotics or supplements as outlined in Example 4 to address the immune disorder.

In alternative embodiments, each or one of the microbes used in the bacterial combination is (at least initially) isolated from a healthy donor or donors, as described in Example 5, or is a genetically modified derivative as described in Example 21, or is a cultured derivative either.

In alternative embodiments, the patient is administered the composition, formulation or pharmaceutical formulation (for example, probiotic) at a dose of between about 10⁵ to 10¹⁵ bacteria, once, twice, three times, or more often per day. Dosing can occur through a lyophilized form, i.e. a lyophilized powder that is given orally to the infant either directly, through a dropper, or through a bottle.

In another embodiment, the infant may be dosed with the composition, formulation or pharmaceutical formulation (for example, probiotic) before, during, and/or immediately after feeding.

In another embodiment, dosing of the composition, formulation or pharmaceutical formulation (for example, probiotic) is continued for 1 month, 6 months, 1 year, or more.

In alternative embodiments, the composition of the infant's gut microbiome and the metabolic and immunological state of the infant gut are used as a measure of successful treatment.

In alternative embodiments, the remission or inhibition of progression of the immune disorder is used as the measure of a successful treatment.

### Example 16: Method of Treating an Infant Based on Stool Biomarkers

This example describes successful therapeutic administration of a live biotherapeutic (or probiotic) as provided herein, which in alternative embodiments comprises one bacteria and a probiotic (or a synbiotic, for example, as set forth in Table 8 or Table 32, below), or alternatively comprises a combination or mix or consortium of bacteria as provided herein, for example, as set forth in Table 1 or Table 4, or live biotherapeutic (also called probiotic) compositions or combinations of bacteria as set forth in Table 2 or Table 30, and/or also comprising administration of at least one prebiotic as provided herein, including one or a combination of prebiotics as provided herein, for example, as set forth in Table 3, to an infant or newborn in need thereof, wherein in alternative embodiments the administration treats or ameliorate a dysbiosis in the infant or newborn, thereby optionally increasing the infant's ability to thrive, or resist a disease or infection.

In alternative embodiments compositions, formulations and pharmaceutical compositions as provided herein, and methods as provided herein are used to treat infants with a dysbiosis. Events that predispose an infant to dysbiosis included but are not limited to: premature birth, extended stay in the neonatal intensive care unit, antibiotic treatment, antibiotic treatment of the mother prior to birth, birth via cesarean section, formula feeding, and known dysbiosis of the mother. To verify the dysbiosis, the infant stool is sampled as described in Example 2 and evaluated in a method comparable to one described in Example 3. Upon indication of dysbiosis based on metabolic, immunological, and microbial biomarkers in the infant stool as defined in Example 3, the infant is administered a live biotherapeutic composition, i.e., a formulation consisting of some combination of microbes as outlined in Example 4 or Example 5 either alone or in combination with prebiotics or supplements as outlined in Example 4 to address the dysbiosis.

In alternative embodiments, each or one of the microbes used in the bacterial combination is (at least initially) isolated from a healthy donor or donors, as described in Example 5, or is a genetically modified derivative as described in Example 21, or is a cultured derivative of either.

In alternative embodiments, the patient is administered the synbiotic at a dose of between about 10⁵ to 10¹⁵ bacteria, once, twice, three times, or more often per day. Dosing can occur through a lyophilized form, i.e. a lyophilized powder that is given orally to the infant either directly, through a dropper, or through a bottle.

In another embodiment, the infant may be dosed with the probiotic before, during, and/or immediately after feeding.

In another embodiment, dosing of the probiotic is continued for 1 month, 6 months, 1 year, or more.

In alternative embodiments, the composition of the infant's gut microbiome and the metabolic and immunological state of the infant gut are used as a measure of successful treatment.

### Example 17: Exemplary Methods of Treating a Child with a Live Biotherapeutic (or Probiotic) for the Treatment and Prevention of Dysbiosis that can Lead to Disease or Reduce Therapeutic Efficacy

This example describes successful therapeutic administration of a live biotherapeutic (or probiotic) as provided herein, which in alternative embodiments comprises one bacteria and a probiotic (or a synbiotic, for example, as set forth in Table 8 or Table 32, below), or alternatively comprises a combination or mix or consortium of bacteria as provided herein, for example, as set forth in Table 2, Example 4, or Table 30, Example 5, and/or administration of prebiotic as provided herein, including a combination of prebiotics as provided herein, for example, as set forth in Table 3, Example 4, to a child in need thereof.

In alternative embodiments compositions, formulations and pharmaceutical compositions as provided herein, and methods as provided herein are used to treat dysbiosis in children, which can be defined as but is not limited to, a high level of pathogenic bacteria, a high level of antibiotic resistance, a metabolic balance that skews away from that of a healthy population, an immunological state that skews away from that of a healthy population, a loss of metabolic function associated with a healthy population, an increase in bacteria associated with a specific disease state, or a microbial population associated with poor therapeutic efficacy.

In alternative embodiments, compositions, formulations and pharmaceutical compositions as provided herein, and methods as provided herein are administered to treat or ameliorate disease states that have been associated with dysbiosis include but are not limited to cancer, diabetes, obesity, allergies, dermatitis, asthma, gout, Alzheimer's disease, Parkinson's disease.

In alternative embodiments, the child is administered a live biotherapeutic composition (or probiotic), for example, a pharmaceutical composition or formulation comprising or consisting of: one bacteria and a prebiotic (or a synbiotic, for example, as set forth in Table 8 or Table 32, below), or alternatively comprises; and/or, one or a combination mix or consortium of microbes as outlined in Table 1, Table 2, Table 4, and Table 30 either alone or in combination with prebiotics or supplements (for example, as outlined in Table 3) to address the dysbiosis and reduce risk of disease or remedy a lack of therapeutic efficacy.

In alternative embodiments, each or one of the microbes used in the bacterial combination is (at least initially) isolated from a healthy donor or donors, as described in Example 5, or is a genetically modified derivative as described in Example 21, or is a cultured derivative of either.

In alternative embodiments, the patient is administered a live biotherapeutic at a dose of between about 10⁵ to 10¹⁵ bacteria, or at a dose of about 10¹⁰, 10¹¹ or 10¹² bacteria total or per dose, which can be in a lyophilized form, for example, or formulated in an enteric coated capsule. In alternative embodiments, the patient takes 1, 2, 3, 4, 5, 6, 7, 8, 9, 10 or 11 or more live biotherapeutic capsules (for example, by mouth or suppository) once, twice or three times or more per day, and the patient can resume a normal diet after about 1, 2, 4, 8, 12, or 24 or more hours.

In another embodiment, the patient may take the live biotherapeutic capsule(s) by mouth before, during, and/or immediately after a meal.

In another embodiment, the patient is given a course of antibiotics before treatment, for example, between one to seven days, or between about one to two weeks prior to the first dose of the live biotherapeutic (for example, as capsule(s)), or three weeks prior, or four weeks prior, or up to 6 months prior to the first dose of live biotherapeutic.

In another embodiment, dosing of the live biotherapeutic (or probiotic) capsule(s) is continued for about 1 month, 6 months, 1 year, or more, or between about one week and 2 years, following termination of a treatment or therapy.

In alternative embodiments, the composition of the child's gut microbiome and the metabolic and immunological state of the child gut are used as a measure of successful treatment.

In alternative embodiments, recovered efficacy of a therapeutic (such as a drug, for example, a cancer therapeutic) is used as a measure of successful treatment.

### Example 17: Exemplary Methods of Treating an Adult with a Live Biotherapeutic (or Probiotic) for the Treatment and Prevention of Dysbiosis that can Lead to Disease or Reduce Therapeutic Efficacy

This example describes successful therapeutic administration of a live biotherapeutic (or probiotic) as provided herein, which in alternative embodiments comprises one bacteria and a probiotic (or a synbiotic, for example, as set forth in Table 8 or Table 32, below), or alternatively comprises a combination or mix or consortium of bacteria as provided herein, for example, as set forth in Table 2, Example 4, or Table 30, Example 5, and/or administration of prebiotic as provided herein, including a combination of prebiotics as provided herein, for example, as set forth in Table 3, Example 4, to an individual in need thereof.

In alternative embodiments compositions, formulations and pharmaceutical compositions as provided herein, and methods as provided herein are used to treat adults with dysbiosis, for example, to treat an adult gut that is dysbiotic or at risk of becoming dysbiotic, which can include a high level of pathogenic bacteria, a high level of antibiotic resistance, a metabolic balance that skews away from that of a healthy population, and immunological state that skews away from that of a healthy population, a loss of metabolic function associated with a healthy population, an increase in bacteria associated with a specific disease state, or a microbial population associated with poor therapeutic efficacy.

In alternative embodiments, compositions, formulations and pharmaceutical compositions as provided herein, and methods as provided herein are administered to treat or ameliorate disease states that have been associated with dysbiosis include but are not limited to cancer, diabetes, obesity, allergies, asthma, dermatitis, gout, Alzheimer's disease, Parkinson's disease.

In alternative embodiments, the adult is administered a live biotherapeutic composition (or probiotic), for example, a pharmaceutical composition or formulation comprising or consisting of: one bacteria and a pre biotic (or a synbiotic, for example, as set forth in Table 8 or Table 32, below), or alternatively comprises; and/or, one or a combination mix or consortium of microbes as outlined in Table 1, Table 2, Table 4, and Table 30 either alone or in combination with prebiotics or supplements (for example, as outlined in Table 3) to address the dysbiosis and reduce risk of disease or remedy a lack of therapeutic efficacy.

In alternative embodiments, each or one of the microbes used in the bacterial combination is (at least initially) isolated from a healthy donor or donors, as described in Example 5, or is a genetically modified derivative as described in Example 21, or is a cultured derivative of either.

In alternative embodiments, the patient is administered a live biotherapeutic at a dose of between about 10⁵ to 10¹⁵ bacteria, or at a dose of about 10¹⁰, 10¹¹ or 10¹² bacteria total or per dose, which can be in a lyophilized form, for example, or formulated in an enteric coated capsule. In alternative embodiments, the patient takes 1, 2, 3, 4, 5, 6, 7, 8, 9, 10 or 11 or more live biotherapeutic capsules (for example, by mouth or suppository) once, twice or three times or more per day, and the patient can resume a normal diet after about 1, 2, 4, 8, 12, or 24 or more hours.

In another embodiment, the patient may take the live biotherapeutic capsule(s) by mouth before, during, and/or immediately after a meal.

In another embodiment, the patient is given a course of antibiotics before treatment, for example, between one to seven days, or between about one to two weeks prior to the first dose of the live biotherapeutic (for example, as capsule(s)), or three weeks prior, or four weeks prior, or up to 6 months prior to the first dose of live biotherapeutic.

In another embodiment, dosing of the live biotherapeutic (or probiotic) capsule(s) is continued for about 1 month, 6 months, 1 year, or more, or between about one week and 2 years, following termination of a treatment or therapy.

In alternative embodiments, the composition of the adult's gut microbiome and the metabolic and immunological state of the adult gut are used as a measure of successful treatment.

In alternative embodiments, recovered efficacy of a therapeutic (such as a drug, for example, a cancer therapeutic) is used as a measure of successful treatment.

### Example 18: Exemplary Methods of Treating Disease Based on Stool Biomarkers

This example describes successful therapeutic administration of a live biotherapeutic (or probiotic) as provided herein, which in alternative embodiments comprises one bacteria and a probiotic (or a synbiotic, for example, as set forth in Table 8 or Table 32, below), or alternatively comprises a combination, mix or consortium of bacteria as provided herein, for example, as set forth in Table 1, Table 2, Table 4, Table 30 and/or administration of prebiotic as provided herein, including a combination of prebiotics as provided herein, for example, as set forth in Table 3, to an individual in need thereof.

In alternative embodiments, compositions, formulations and pharmaceutical compositions as provided herein, and methods as provided herein, are administered to treat or ameliorate an adult having a condition or disease in which the microbiome has been implicated. These diseases include but are not limited to for example: cancer, diabetes, obesity, allergies, asthma, gout, Alzheimer's disease, Parkinson's disease.

The patient's stool is collected and analyzed using t′e methods described in Example 3. In one embodiment, whole genome sequencing is performed and the presence of microbes that are characteristic of healthy individuals or diseased individuals is evaluated. Based on the abundance profiles of healthy individuals and diseased individuals, a classifier is developed to predict if any given microbiome composition represents a healthy or diseased patient. This may be based on the amount of one or more particular organisms present or other criteria that combine aspects of the whole genome sequence data. This classifier is applied to the patient's microbiome composition, to predict whether the patient needs live biotherapeutic intervention.

In another embodiment, metabolomics is performed on the stool or plasma; a classifier is developed based on concentrations of one or more metabolites in all patient data collected to date as well as the composition of their microbiome. This classifier is applied to the patient's data to predict whether the patient needs live biotherapeutic intervention.

In another embodiment, immunological analysis is performed on the stool or plasma; a classifier is developed based on concentrations of one or more immunological markers in all patient data collected to date as well as the composition of their microbiome. This classifier is applied to the patient's data to predict whether the patient needs live biotherapeutic intervention. If the patient is classified as requiring intervention, a live biotherapeutic will be administered to shift the microbiome away from the dysbiosis associated with the disease state.

In alternative embodiments, each or one of the microbes used in the bacterial combination is (at least initially) isolated from a healthy donor or donors, as described in Example 5, or is a genetically modified derivative as described in Example 21, or is a cultured derivative of either.

In alternative embodiments, the patient is administered a live biotherapeutic at a dose of between about 10⁵ to 10¹⁵ bacteria, or at a dose of about 10¹⁰, 10¹¹ or 10¹² bacteria total or per dose, which can be in a lyophilized form, for example, or formulated in an enteric coated capsule. In alternative embodiments, the patient takes 1, 2, 3, 4, 5, 6, 7, 8, 9, 10 or 11 or more live biotherapeutic capsules (for example, by mouth or suppository) once, twice or three times or more per day, and the patient can resume a normal diet after about 1, 2, 4, 8, 12, or 24 or more hours.

In another embodiment, the patient may take the live biotherapeutic capsule(s) by mouth before, during, and/or immediately after a meal.

In another embodiment, the patient is given a course of antibiotics before treatment, for example, between one to seven days, or between about one to two weeks prior to the first dose of the live biotherapeutic (for example, as capsule(s)), or three weeks prior, or four weeks prior, or up to 6 months prior to the first dose of live biotherapeutic.

In another embodiment, dosing of the live biotherapeutic capsule(s) is continued 1 month, 6 months, 1 year, or more, or between about one week and 2 years, following termination of the treatment.

In alternative embodiments, the composition of the adult's gut microbiome and the metabolic and immunological state of the adult gut are used as a measure of successful treatment.

### Example 19: Exemplary Methods of Treating an Expectant Mother with a Live Biotherapeutic (or Probiotic) for the Treatment and Prevention of Dysbiosis that can Lead to Maternal or Infant Disease

This example describes administration of a live biotherapeutic (or probiotic) as provided herein, which in alternative embodiments comprises one bacteria and a probiotic (or a synbiotic, for example, as set forth in Table 8 or Table 32, below), or alternatively comprises at least one or a combination, mix or consortium of bacteria as provided herein, for example, as set forth in Table 1, Table 4, Table 2, or Table 30 and/or administration of prebiotic as provided herein, including a combination of prebiotics as provided herein, for example, as set forth in Table 3, to an individual in need thereof.

In alternative embodiments, compositions, formulations and pharmaceutical compositions as provided herein, and methods as provided herein, are administered to treat or ameliorate an expectant mother's, or individual expecting pregnancy, gut when the gut is dysbiotic or at risk of becoming dysbiotic. For example, the treated dysbiosis could be the presence of pathogenic bacteria, for example, having a high level of pathogenic bacteria, a high level of antibiotic resistance, a metabolic balance that skews away from that of a healthy population, and immunological state that skews away from that of a healthy population, a loss of metabolic function associated with a healthy population, or an increase in bacteria associated with adverse events for a mother and her child. Dysbiosis could be caused by any number of lifestyle factors, including but not limited to the mother's birth mode, maternal antibiotic usage, maternal diet, and maternal GI conditions.

In alternative embodiments, the expectant mother is administered a live biotherapeutic composition, i.e., a formulation consisting of some combination, mix or consortium of microbes as outlined in Table 4, or as listed in Table 1, Table 2 or Table 30, either alone or in combination with prebiotics or supplements as outlined in Table 3, to address the dysbiosis with the goal of preventing maternal or infant disease.

In alternative embodiments, each or one of the microbes used in the bacterial combination, mix or consortium is (at least initially) isolated from a healthy donor or donors, as described in Example 5, or is a genetically modified derivative as described in Example 21, or is a cultured derivative of either.

In alternative embodiments, the patient (for example, the expectant mother) is administered a live biotherapeutic (or probiotic) as provided herein at a dose of between about 10⁵ to 10¹⁵ bacteria, or at a dose of about 10¹⁰, 10¹¹ or 10¹² bacteria total or per dose, which can be in a lyophilized form, for example, or formulated in an enteric coated capsule. In alternative embodiments, the patient takes 1, 2, 3, 4, 5, 6, 7, 8, 9, 10 or 11 or more live biotherapeutic capsules (for example, by mouth or suppository) once, twice or three times or more per day, and the patient can resume a normal diet after about 1, 2, 4, 8, 12, or 24 or more hours.

In another embodiment, the patient may take the live biotherapeutic capsule(s) by mouth before, during, and/or immediately after a meal.

In another embodiment, the patient is given a course of antibiotics before treatment, for example, between one to seven days, or between about one to two weeks prior to the first dose of the live biotherapeutic (for example, as capsule(s)), or three weeks prior, or four weeks prior, or up to 6 months prior to the first dose of live biotherapeutic.

In another embodiment, dosing of the live biotherapeutic capsule(s) is continued 1 month, 6 months, 1 year, or more, or between about one week and 2 years, following termination of the treatment.

In alternative embodiments, the composition of the adult's gut microbiome and the metabolic and immunological state of the adult gut are used as a measure of successful treatment.

### Example 20: Exemplary Methods of Treating a Cancer Patient with a Live Exemplary Biotherapeutic to Reduce Dysbiosis

This example describes administration of a live biotherapeutic (or probiotic) as provided herein, which in alternative embodiments comprises one bacteria and a probiotic (or a synbiotic, for example, as set forth in Table 8 or Table 32, below), or alternatively comprises, a combination, mix or consortium of bacteria as provided herein, for example, as set forth in Table 1, Table 2, Table 30 or Table 4, and/or administration of prebiotic as provided herein, including one or a combination of prebiotics as provided herein, for example, as set forth in Table 3, to an individual in need thereof.

In alternative embodiments compositions, formulations and pharmaceutical compositions and provided herein, and methods as provided herein are administered to a patient suffering from cancer, and the formulation or a pharmaceutical composition can comprise a combination, mix or consortium of microbes (for example, bacteria) as provided herein (for example as listed in Table 2, Table 30, Table 1 or Table 4) either in monotherapy or in combination with chemotherapy, radiation therapy, a checkpoint inhibitor, a Chimeric Antigen Receptor (CAR) T-cell therapy (CAR-T) or other immunotherapy or cancer treatment, and the patient can be administered the live biotherapeutic for the duration of treatment or for only one or several segments of treatment.

In alternative embodiments, each or one of the microbes used in the bacterial combination, mix or consortium is (at least initially) isolated from a healthy donor or donors, as described in Example 5, or is a genetically modified derivative as described in Example 21, or is a cultured derivative of either.

In alternative embodiments, the patient is administered a live biotherapeutic (or probiotic) as provided herein at a dose of between about 10⁵ to 10¹⁵ bacteria, or at a dose of about 10¹⁰, 10¹¹ or 10¹² bacteria total or per dose, which can be in a lyophilized form, for example, or formulated in an enteric coated capsule. In alternative embodiments, the patient (for example, expectant mother, or new mother) takes 1, 2, 3, 4, 5, 6, 7, 8, 9, 10 or 11 or more live biotherapeutic (or probiotic) capsules as provided herein (for example, by mouth or suppository) once, twice or three times or more per day, and the patient can resume a normal diet after about 1, 2, 4, 8, 12, or 24 or more hours.

In another embodiment, the patient may take the live biotherapeutic (or probiotic) capsule(s) by mouth before, during, and/or immediately after a meal.

In another embodiment, the patient is given a course of antibiotics before treatment, for example, between one to seven days, or between about one to two weeks prior to the first dose of the live biotherapeutic (for example, as capsule(s)), or three weeks prior, or four weeks prior, or up to 6 months prior to the first dose of live biotherapeutic.

In another embodiment, dosing of the live biotherapeutic, for example, as capsule(s), is started one to seven days, or one to two weeks, prior to administration of a first dose of a chemotherapy, a first checkpoint inhibitor dose, start of a CAR-T therapy or any immunotherapy or cancer therapy.

In another embodiment, dosing of the live biotherapeutic (or probiotic) capsule(s) is continued 1 month, 6 months, 1 year, or more, or between about one week and 2 years, following termination of the treatment, for example, checkpoint inhibitor administration, chemotherapy or any immunotherapy.

In alternative embodiments, patient response to the combination, mix or consortium bacterial therapy as provided herein is a measure of success and for solid tumors is based on radiographic assessment using the Response Evaluation Criteria in Solid Tumors (RECIST 1.1) criteria (Schwartz, et al. (2016) Eur. J. Cancer. 62:132-137) at 6 months after treatment initiation, and again after 12 months and 24 months. Patients are classified as complete responders if all target lesions are gone, partial responders if there is at least 30% reduction in the sum of diameters of all target lesions, progressive disease if there is at least 20% increase in the sum of diameters of all target lesions, and stable disease otherwise. For blood cancers, the Response Evaluation Criteria in Lymphoma (RECIL) criteria is used, based on [18F]2-fluoro-2-deoxy-D-glucose positron-emission tomography (FDG-PET) (Younes, A. et al (2017) Ann. Oncol. 28:1436-1447).

### Example 21: Genetic Modification of Live Biotherapeutic Microbes

Microbes of interest, including microbes as provided herein, for example, as listed in Table 1, Table 2, Table 4, Table 8, Table 30, or Table 32 including bacteria from all the genera listed therein, and including the combinations of microbes as provided herein, for example, the exemplary combinations 1 to 122 as described in Table 2, or as identified from the in vivo and ex vivo analyses described in Example 7 and Example 8, are interrogated or investigated to identify mechanisms of action, and the discovered mechanisms are leveraged using a genetic modification or modifications to amplify the microbe's therapeutic effect.

In alternative embodiments, this is accomplished in two stages. First, complementary bioinformatic and experimental approaches are used to identify the genes within a microbe of interest responsible for its therapeutic effect. Second, synthetic biology techniques are used to engineer over-expression of the identified genes within the original organism of discovery or inserted for overexpression in the genome of a chassis organism. Chassis organisms include any microbe as described herein, including genera of bacteria as provided herein, and also include bacteria as listed in Table 2, including Bacillus subtilis, Escherichia coli Nissle, Bacteroides fragilis, Bacteroides thetaiotaomicron, Bifidobacterium infantis, Bifidobacterium bifidum, Bifidobacterium longum, Bifidobacterium breve, Bifidobacterium pseudocatenulatum, or any microbes listed in the combinations, mixes or consortiums as provided herein in Table 2, Table 8, Table 30, Table 32, or Table 4, or the original organism of interest itself.

In alternative embodiments, bacteria that have adequate genetic tools may be modified through restriction nucleases, zinc finger enzymes, CRISPR, and other techniques designed for genetic modification. These techniques vary from bacteria to bacteria (Liu Y. et al. (2022) Frontiers in Microbiology 13).

In alternative embodiments, bacteria that lack adequate genetic tools may first be evaluated to improve the available toolkit and facilitate complex synthetic biology. For example, expression levels in Bacteroides fragilis and Bacteroides thetaiotaomicron are evaluated to determine optimal promoters, ribosomal binding sites, and terminators to improve control over constructs developed from the two bacteria. In an alternate example, expression levels in Bifidobacterium infantis, Bifidobacterium breve, Bifidobacterium bifidum, and Bifidobacterium longum are evaluated in the context of human milk oligosaccharides and/or other carbohydrates to determine optimal promoters, ribosomal binding sites, and terminators for control of gene expression upon introduction of human milk oligosaccharides and/or other carbohydrates.

In alternative embodiments, the carbohydrate utilization loci of a given bacteria may be transferred to another bacteria to improve growth and control engraftment of that bacteria. For example, the human milk oligosaccharide utilization clusters from Bifidobacterium infantis may be transferred into Bacteroides fragilis or Bacteroides thetaiotaomicron to better control their engraftment in the presence of human milk oligosaccharides which are absent from the diet of adults and most formula feeding children.

In alternative embodiments, the carbohydrate utilization loci of a given bacteria may be transferred to another bacteria to improve growth, control engraftment, and control spatial organization of that bacteria. For example, mucin utilization genes can be transferred from mucin utilizers such as Akkermansia muciniphila into bacteria that do not consume mucin to increase engraftment and better control localization in the gut.

In alternative embodiments, genes are incorporated to control bacterial growth under the control of inducible promoters, for instance a toxin-antitoxin system in which the anti-toxin is only generated in the presence of a specific inducer.

In alternative embodiments, bacteria are genetically modified to make postbiotics like urolithin A from ellagic acid or indole-3-lactate from tryptophan. These postbiotics can be produced in situ in the gut or can be produced and isolated in vitro for formulation along with bacteria of interest.

In alternative embodiments, bacteria are genetically modified to produce complex oligosaccharides that can serve as prebiotics for desired strains. These prebiotics can be produced in situ in the gut or can be produced and isolated in vitro for formulation along with bacteria of interest.

In alternative embodiments, microbes as provided herein are genetically modified to increase expression of existing therapeutically effective genes, or to install extra copies of these genes, or to install into a microbe lacking these functions any one of these genes. Methods for genetic engineering/augmenting a microbe of interest, for example, a gut microbe, to alter expression of existing therapeutically effective genes or to install extra copies of said genes or to install said genes in a microbe lacking these functions are numerous in the art. Techniques applied to gut microbes and related organisms for experimental gene disruption, gene replacement or gene expression modulation include CRISPR-Cas9 genome editing (Bruder et al (2016) Applied and Environmental Microbiology 82:6109-6119) (Pan et al. (2022) PNAS), bacterial conjugation (Cuiv et al (2015) Nature Scientific Reports 5:13282; Ronda et al. (2019) Nature Methods 16:167-170), gene replacement mutagenesis by homologous recombination (Cartman et al (2012) Applied Environmental Microbiology 78:4683-4690; Heap et al (2007) Journal of Microbiological Methods 70:452-464), random transposon mutagenesis (Cartman and Minton (2010) Applied Environmental Microbiology 76:1103-1109), and antisense-based gene expression attenuation (Forsyth et al (2002) Molecular Microbiology 43:1387-1400; Kedar et al (2007) Antimicrobial Agents and Chemotherapy 51:1708-1718.

Genes of interest inserted into microbes as provided herein, or whose expression is increased in microbes as provided herein, can be engineered to immediately follow and be under inducible control by various promoter elements that are functional in gut microbes. Highly inducible and controllable promoter elements are available for bacteria in the gram-negative genus Bacteroides (Lim et al (2017) Cell 169:547-558; Bencivenga-Barry et al (2019) Journal of Bacteriology doi: 10.1128/JB.00544-19). Some of these are responsive to various diet-derived polysaccharides, while those often most useful for use for inducible function determination in animal models such as mice rely on induction by tetracycline derivatives like anhydrotetracycline at sub-bactericidal levels. Anhydrotetracycline can be employed as an inducer for engineered promoters in gut Clostridia (Dembek et al (2017) Frontiers of Microbiology 8:1793). Promoters that respond to bile acids are identified in gram-positive gut Clostridium species (Wells and Hyemon (2000) Applied Environmental Microbiology 66:1107-1113) and in Eubacterium species (Mallonee et al. (1990) Journal of Bacteriology 172:7011-7019. Also, inducible promoters that respond to sugars such as lactose (Banerjee et al (2014) Applied Environmental Microbiology 80-2410-2416) and arabinose (Zhang et al (2015) Biotechnology for Biofuels 8:36) are identified and useful in related Clostridial species. Genes inserted in exemplary recombinant bacterium can be induced under low-oxygen conditions from promoters driven by transcriptions factors such as FNR (fumarate and nitrate reductase) (Oxer et al (1991) Nucleic Acids Research, 19, 11:2889-2892). Genes of interest inserted in microbes as provided herein can also be engineered to immediately follow and be under constitutive control by various promoter elements that are functional in gut microbes. Constitutive promoter libraries and promoter-RBS (ribosome binding site) pairs have been created for bacteria in the gram-negative genus Bacteroides (Mimee et al (2015) Cell Syst. 1, 62-71) and computational models have been developed from Bacillus subtilis promoter sequences data sets for promoter prediction in Gram-positive bacteria (Coelho et al (2018) Data Br. 19, 264-270).

### Engineering of Metabolic Pathways in Live Biotherapeutics

In one embodiment, an organism (for example, a bacteria) used to practice embodiments as provided herein is genetically modified to overexpress a pathway for production of any short chain fatty acid (SCFA), including butyrate or butyric acid, propionate and acetate. Butyric acid is naturally produced in many gut microorganisms and is derived from two molecules of acetyl-CoA, a central metabolic intermediate that is ubiquitous in microorganisms. In one embodiment, the native pathway is overexpressed, for example, as discussed herein. In another embodiment, a heterologous pathway is constructed by introducing one or more genes from a different organism, including all genes derived from different organisms. Condensation of two acetyl-CoA molecules is catalyzed by a ketothiolase (EC:2.3.1.9), such as the atoB gene from Escherichia coli, to produce one molecule of acetoacetyl-CoA (Sato et al. (2007) J. Biosci. Bioengineer. 103:38-44). Alternative candidates are obtained by Basic Local Alignment Search Tool (BLAST) search of this sequence (Altschul et al. (1997) Nuc. Acids. Res. 25:3389-3402), obtaining homologous genes either known or predicted to encode similar enzyme function. Exemplary gene candidates are obtained using the following GenBank accession numbers.

| | | |
|---|---|---|
| atoB | Escherichia coli | NP_416728.1 |
| yqeF | Escherichia coli | NP_417321.2 |
| phaA | Cupriavidus necator | YP_725941 |
| bktB | Cupriavidus necator | AAC38322.1 |
| thiA | Clostridium acetobutylicum | NP_349476.1 |
| thiB | Clostridium acetobutylicum | NP_149242.1 |

The second step in the pathway involves reduction of acetoacetyl-CoA to 3-hydroxybutyryl-CoA by a hydroxyacyl-CoA dehydrogenase (EC:1.1.1.35), such as that encoded by hbd in Clostridium acetobutylicum (Atsumi et al. (2008) Metab. Eng. 10(6): 305-311). Similarly, to above, alternate candidates are identified in the literature or by BLAST. Exemplary candidates are as follows.

| | | |
|---|---|---|
| paaH | Escherichia coli | NP_415913.1 |
| hbd | Clostridium acetobutylicum | NP_349314.1 |
| hbd | Pseudomonas putida | KT2440 NC_002947.4 |
| RSP_3970 | Rhodobacter sphaeroides 2.4.1 | YP_345236.1 |

The next step is the dehydration of 3-hydroxybutyryl-CoA to crotonyl-CoA by an enoyl-CoA hydratase, also known as crotonase (EC:42.1.55), such as that encoded by the crt gene of Clostridium acetobutylicum (Kim et al. (2014) Biochem. Biophys. Res. Commun. 451:431-435) or the homologs listed below.

| | | |
|---|---|---|
| Crt | Clostridium acetobutylicum | NC_003030.1 |
| echA18 | Mycobacterium bovis AF2122/97 | NC_002945.4 |
| maoC | Escherichia coli | NP 415905.1 |
| crt | Bacillus thuringiensis | NC_005957.1 |

Next, crotonyl-CoA is reduced to butyryl-CoA through the action of an enoyl-CoA reductase (EC:1.3.1.38 or EC:1.3.1.44), such as that encoded by the bcd gene of Clostridium acetobutylicum (Boynton et al. (1996) J. Bacteriol. 178:3015-3024). Activity of this enzyme can be enhanced by expressing bcd in conjunction with expression of the C. acetobutylicum etfAB genes, which encode an electron transfer flavoprotein. Several eukaryotic enzymes with this activity have also been identified, such as TER from Euglena gracilis, that upon removal of the mitochondrial targeting leader sequence have demonstrated superior activity in E. coli (Hoffmeister et al. (2005) J. Biol. Chem. 280:4329-4338). Protein sequences for these and other exemplary sequences can be obtained using the following GenBank accession numbers.

| | | |
|---|---|---|
| Bcd | Clostridium acetobutylicum | NP 34.9317.1 |
| etfA | Clostridium acetobutylicum | NP 349315.1 |
| etfB | Clostridium acetobutylicum | NP 349316.1 |
| TER | Euglena gracilis | Q5EU90.1 |
| TDE0597 | Treponema denticola | NP 97.1211.1 |

The final step of this pathway is CoA removal from butyryl-CoA to generate butyric acid. Although numerous CoA hydrolases occur in most bacteria, for example, tesS from E. coli ((Naggert et al. (1991) J. Biol. Chem. 266:11044-11050), it is desirable to recover energy from hydrolysis of the thioester bond in the form of ATP. The sucCD complex of E. coli (EC:6.2.1.5) is one example of this, known to catalyze the conversion of succinyl-CoA and ADP to succinate and ATP (Buck et al. (1985) Biochem. 24:6245-6252). Another example is sucD, succinic semialdehyde dehydrogenase, from Porphyromonas gingivalis (Yim et al. (2011) Nat. Chem. Biol. 7:445-452). Another option, using phosphotransacetylase/butyrate kinase (EC:2.3.1.19, EC:2.7.2.7), is catalyzed by the gene products of buk1, buk2, and ptb from C. acetobutylicum (Walter et al. (1993) Gene 134:107-111) or homologs thereof. Finally, an acetyltransferase capable of transferring the CoA group from butyryl-CoA to acetate can be applied (EC:2.8.3.9), such as Cat3 from C. kluyveri (Sohling and Gottschalk (1996) J. Bacteriol. 178:871-880). Protein sequences for these and other exemplary sequences can be obtained using the following GenBank accession numbers.

| | | |
|---|---|---|
| Ptb | Clostridium acetobutylicum | NP 349676 |
| buk1 | Clostridium acetobutylicum | NP 349675 |
| buk2 | Clostridium acetobutylicum | Q97II1 |
| sucC | Escherichia coli | NP_415256.1 |
| sucD | Escherichia coli | AAC73823.1 |
| cat3 | Clostridium kluyveri | EDK35586.1 |
| tesB | Escherichia coli | NP_414986 |

In another embodiment, a microbe used to practice embodiments as provided herein is genetically modified to metabolize bile acids, also referred to as bile salts to indicate the predominant form at neutral pH, that are produced in the liver and present in the gut at about 1 mM concentration. Two such types of bile acid conversion processes are catalyzed by bacteria. The first is deconjugation, which removes either taurine or glycine that is frequently found conjugated to bile acids (Ridlon et al. (2016) Gut Microbes 7:22-39; Masuda et al. (1981) Microbiol. Immunol. 25:1-11). This is catalyzed by bile salt hydrolase (BSH) enzymes (EC:3.5.1.24), which are widespread in many gut bacteria. Some BSHs have broad substrate specificity, while others are very specific for a particular bile salt. The substrate range of a BSH of interest is determined by assay of purified BSH or crude lysates from the native host, on a panel of glycine and taurine conjugated bile salts (Jones et al. (2008) Proc. Nat. Acad. Sci. USA 105:13580-13585). To enhance the activity and substrate range of bile salt deconjugation in the engineered microbe, native BSHs of interest and/or heterologous genes from other microbes are introduced. Exemplary genes are listed below. Still others are found by GenBank search or BLAST of these sequences to identify homologs.

| | | |
|---|---|---|
| Bsh | Bifidobacterium longum | AF148138.1 |
| bsh | Bifidobacterium animalis | AY530821.1 |
| bsh | Enterococcus faecalis | GG688660.1 |
| bsh3 | Lactobacillus plantarum | ACL98170.1 |
| cbh2 | Bacteroides vulgatis | RIB33278.1 |
| cbah | Clostridium butyricum | EEP54620.1 |

The other type of bile acid metabolism introduced into a microbe used to practice embodiments as provided herein is capable of converting primary to secondary bile acids, which entails removal of the 7-alpha-hydroxy or 7-beta hydroxy group from the primary bile acid; for example, the conversion of cholic acid to deoxycholic acid or chenodeoxycholic acid to lithocholic acid. The archetype pathway for this process is encoded by the bai gene cluster in Clostridium scindens (Coleman et al. (1987) J. Bacteriol. 169:1516-1521; Ridlon et al. (2006) J. Lipid. Res. 47:241-259) and has been well characterized. In addition, a functional C. scindens dihydroxylation was established in Clostridium sporogenes (Funabashi et al. (2019) BioRxiv). The first step is a bile acid-CoA ligase (baiB, EC:6.2.1.7) to activate the molecule for the subsequent reaction steps. Next, an alcohol dehydrogenase (baiA, EC:1.1.1.395) oxidizes the 3-hydroxyl to a keto group. An NADH: flavin oxidoreductase then introduces a double bond into the ring by either baiCD (EC:1.3.1.115) or baiH (EC:1.3.1.116), depending on the substrate. The coA is then removed or transferred to another primary bile acid by a CoA transferase (baiF, EC:2.8.3.25). The 7-alpha or 7-beta-hydroxy group is then removed by a dehydratase (baiE or bail, respectively, EC:4.2.1.106) to form a second double bond in a conjugated position to the other one. Enzymes encoded by baiH and baiCD then serve to reduce the double bonds consecutively, and finally the alcohol dehydrogenase reduces the 3-keto back to a hydroxyl. High bile acid dihydroxylation activity has also been observed in Eubacterium sp. Strain VPI 12708, Eubacterium sp. Strain Y-1113, Eubacterium sp. Strain I-10, Eubacterium sp. Strain M-18, Eubacterium sp. Strain TH-82, Clostridium sp. Strain TO-931, and Clostridium sp. Strain HD-17. Homologs for some of the bai genes have been identified in these organisms (Doemer et al. (1997) Appl. Environ. Microbiol. 63:1185-1188), and thus represent alternate gene candidates. Homologs of all essential genes for pathway function were also identified in Clostridium hylemonde DSM 15053, Dorea sp. D7, and a novel Firmicutes bacterium (Das et al. (2019) BMC Genomics 20:517).

To introduce the conversion pathway into the genetically modified host, the following C. scindens genes or suitable homologs are expressed: baiA, baiB, baiCD, baiE, baiF, and baiH. In some embodiments, the baiG gene, encoding a transporter, is also expressed. In other embodiments, the bail gene predicted to encode a delta-5-ketoisomerase, is introduced in order to enable dihydroxylation of secondary bile acids requiring this step.

Tryptophan derivatives are produced by many microbes, including gut bacteria, and have been implicated in strengthening the epithelial cell barrier and modulating the expression of pro-inflammatory genes by T cells in the GI tract (Bercik et al. (2011) Gastroenterology 141:599-609). A gut microbe is engineered to overexpress one or more tryptophan derivatives by either overexpressing native genes or introducing heterologous genes described below.

In one embodiment, a microbe used to practice embodiments as provided herein is engineered to convert tryptophan to indole by introduction of a tryptophanase, such as that encoded by the tnaA gene of E. coli (Li and Young (2013) Microbiology 159:402-410). Other candidates are found by literature search or BLAST of the sequence to find homologs, as exemplified by the following:

| | | |
|---|---|---|
| tnaA | Escherichia coli | NP_415256.1 |
| tnaA | Bacteroides thetaiotamicron | NP_810405.1 |
| tnaA | Vibrio tasmaniensis | LGP32 VS_RS05915 |
| tnaA | Treponema denticola | TDE0251 |

In another embodiment, a microbe used to practice embodiments as provided herein is engineered to convert tryptophan to indoleacetate. This pathway begins with a tryptophan aminotransferase (EC:2.6.1.27) such as that encoded by the Tam1 gene of Ustilago maydis (Zuther et al. (2008) Mol. Microbiol. 68:152-172), which uses a-ketoglutarate as the amino acceptor and produces indolepyruvate. Although a microbial sequence for this enzyme is not currently in GenBank, activity has been reported in Clostridium sporogenes (O'Neil et al. (1968) Arch. Biochem. Biophys. 127:361-369). Alternatively, a deaminating tryptophan oxidase (EC:1.3.3.10) such as that encoded by the vioA gene of Chromobacterium violaceum (August et al. (2000) J. Mol. Microbiol. Biotechnol. 2:513-519) uses molecular oxygen to oxidize and deaminate tryptophan to produce indolepyruvate. Alternative candidates include those indicated as follows:

| | | |
|---|---|---|
| vioA | Chromobacterium violaceum | CV_RS16140 |
| WP_133678757 | Paludibacterium purpuratum | WP_133678757.1 |
| WP_034786442 | Janthinobacterium lividum | WP_034786442.1 |

The next gene to be introduced encodes an indolepyruvate decarboxylase (EC:4.1.1.74), which produces indole-3-acetaldehyde from indolepyruvate. An example is the ipdC gene from Enterobacter cloacae (Koga et al. (1991) Mol. Gen. Genet. 226:10-16). Other exemplary genes can be accessed by the GenBank accession numbers listed below:

| | | |
|---|---|---|
| ipdC | Enterobacter cloacae | WP_013098183.1 |
| CFNIH1_{—} | Citrobacter freundii | CFNIH1_RS23020 |
| RS23020 | | |
| ipdC | Rhodopseudomonas palustris | CGA009 TX73_RS15890 |
| ipdC | Azospirillum brasilense | AMK58_RS11560 |

Indole-3-acetaldehyde is then oxidized to indoleacetate by an aldehyde dehydrogenase (EC:1.2.1.3), such as that encoded by the aldA gene of Pseudomonas syringae (McClerklin et al. (2018) PloS Pathog. 14: e1006811). Numerous aldehyde dehydrogenases exist, though the best candidates are those homologous to this aldA or others with known activity on indole-3-aldehyde or similar molecules. Exemplary gene candidates can be accessed by the GenBank accession numbers listed below:

| | | |
|---|---|---|
| aldA | Pseudomonas syringae | PSPTO_0092 |
| CFNIH1_{—} | Citrobacter freundii | CFNIH1_RS23020 |
| RS23020 | | |
| WP_{—} | Pseudomonas coronafaciens | WP_005887684.1 |
| 005887684.1 | | |
| SPOG_02634 | Schizosaccharomyces cryophilus | OY26 SPOG_02634 |

In another embodiment, a tryptophan decarboxylase (EC:4.1.1.28) is introduced into a microbe used to practice embodiments as provided herein to produce tryptamine. This activity is rare among bacteria, but two such enzymes have recently been identified: CLOSPO_02083 from Clostridium sporogenes and RUMGNA_01526 from Ruminococcus gnavus (Williams et al. (2014) Cell Host Microbe 16:495-503).

In another embodiment, the pathway to produce indole-3-lactate (ILA) is introduced into the genetically modified microbe. ILA plays an important role in gut immune development in infants and is thought to have an overall antiinflammatory effect (Laursen et al. (2021) Nature Microbiology 6:1367-1382). It is known to be synthesized by a small number of gut bacteria through aromatic lactate dehydrogenases (Dodd et al. (2017) Nature 551:648-652). The enzyme for conversion from the precursor indolepyruvate, synthesized as described above, has been identified in Clostridium sporogenes ATCC 15579 (fldH) and Bifidobacterium infantis DSM20088 (ALDH). Homologs of these genes in other microbes are also candidates for expression, found by BLAST of the C. sporogenes gene or the B. infantis gene.

In another embodiment, the pathway to produce indole propionate (IPA) is introduced into the genetically modified microbe. IPA has been implicated in intestinal barrier fortification by engaging the pregnane X receptor (Venkatesh et al. (2014) Immunity 41:296-310) and is known to be synthesized by a small number of gut bacteria (Elsden et al. (1976) Arch. Microbiol. 107:283-188). The genes encoding this pathway have been discovered in Clostridium sporogenes, enabling a pathway to be proposed. Indole-3-lactate, synthesized as described above, is dehydrated to produce indole acrylate. Indoleacrylate is then reduced to IPA by an acyl-CoA dehydrogenase. These are encoded by the fldBC and acdA genes in C. sporogenes, respectively (Dodd et al. (2017) Nature 551:648-652). Homologs of these genes in other microbes are also candidates for expression, found by BLAST of the C. sporogenes genes.

In another embodiment, a microbe used to practice embodiments as provided herein is engineered to consume a sugar or polysaccharide, for example, a cellobiose, which is a reducing sugar consisting of two β-glucose molecules linked by a B (1->4) bond that is recalcitrant to catabolismby most gut microbes. Consumption of cellobiose first requires a specific enzyme II complex (EC:2.7.1.205) of the phosphotransferase system (PTS), such as the celABC operon in E. coli (Keyhani et al. (2000) J. Biol. Chem. 275:33091-33101). When expressed in a heterologous host, this component functions together with the native PTS machinery to import and phosphorylate cellobiose to generate cellobiose-6-phosphate. Alternate candidates for this step are listed below:

| | | |
|---|---|---|
| celA | Enterococcus gilvus | WP_10781765.1 |
| celB | Enterococcus gilvus | WP_010780456.1 |
| celC | Enterococcus gilvus | WP_010780458.1 |
| celA | Lactococcus lactis subsp. Lactis | NP_266573.1 |
| celB | Lactococcus lactis subsp. Lactis | NP_266330.1 |
| ptcA | Lactococcus lactis subsp. Lactis | NP_266570.1 |
| celB | Bacillus coagulans | BF29_RS14550 |

A 6-phospho-beta-glucosidase (EC:3.2.1.86) is then required to convert the cellobiose-6P into one molecule of glucose and one molecule of glucose-6-P, both of which are readily used by the host. An example is the 6-phospho-beta-glucosidase from Bacillus coagulans, which has successfully been expressed in E. coli (Zheng et al. (2018) Biotechnology for Biofuels 18:320). Alternate candidates are listed below:

| | | |
|---|---|---|
| celA | Enterococcus gilvus | WP_10781765.1 |
| celB | Enterococcus gilvus | WP_010780456.1 |
| celC | Enterococcus gilvus | WP_010780458.1 |
| celA | Lactococcus lactis subsp. Lactis | NP_266573.1 |
| celB | Lactococcus lactis subsp. Lactis | NP_266330.1 |
| ptcA | Lactococcus lactis subsp. Lactis | NP_266570.1 |
| celB | Bacillus coagulans | BF29_RS14550 |

In another embodiment, a microbe used to practice embodiments as provided herein is genetically modified by deleting or reducing expression of genes to eliminate or reduce production of metabolites, such as the polyamines putrescine, spermidine, and cadaverine. These molecules are essential for gastrointestinal mucosal cell growth and function, but excess of these compounds has been linked to gut dysbiosis and poor nutrient absorption (Forget et al. (1997) J. Pediatr. Gastroenterol. Nutr. 24:285-288).

The primary routes for polyamine synthesis in bacteria are decarboxylation of the amino acid's arginine or ornithine. Ornithine decarboxylase (ODC, EC:4.1.1.17) converts ornithine to putrescine, while arginine decarboxylase (ADC, EC:4.1.1.19) converts arginine to agmatine, which is subsequently converted to putrescine by agmatinase (EC:3.5.3.11). Putrescine can then be converted to other derivatives such as spermidine. Therefore, a reduction in ODC and/or ADC expression will reduce polyamine production in the host microbe. E. coli contains two ODC isomers, encoded by the speC and speF genes, as well as two isomers of ADC encoded by speA and adiA. BLAST searches using these sequences, or other known bacterial ODC and ADC genes, applied to the genome of the organism of interest is used to identify genes encoding these functions in the organism to be genetically modified. One or both of these genes, or homologs thereof, are then deleted from the host genome using tools such as lambda-red mediated recombination (Datsenko and Wanner (2000) Proc. Nat. Acad. Sci. USA 97:6640-6645), CRISPR-Cas9 genome editing (Bruder et al (2016) Appl. Environ. Microbiol. 82:6109-6119), or any other method resulting in the removal of genes or portions of genes from the chromosome. In another embodiment, these methods are used to replace the native promoters of these genes with alternate promoters of different strengths, or to modify the ribosome binding site, resulting in reduced production of the ODC and ADC enzymes. In yet another embodiment, expression is reduced through a gene silencing mechanism such as antisense RNA-based attenuation (Nakashima et al. (2012) Methods Mol. Biol. 815:307-319) or CRISPR interference (Choudhary et al. (2015) Nat. Comm. 6:6267).

### Engineering of Peptide Expression in Therapeutic Microbes

In one embodiment, a microbe is engineered to produce a recombinant peptide or protein for therapeutic purposes. This peptide or protein may see improved therapeutic efficacy from microbial expression due to improved stability or bioavailability. This peptide or protein may be a novel protein or a protein with known therapeutic benefits.

In another embodiment, the recombinantly produced peptide is GLP-1 (sequence: His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg) (SEQ ID NO: 300) or a GLP-1 mimic. This class of peptides is known to help control diabetes and instigate weight loss. Production of these peptides in the gut serves to improve bioavailability and stability of the peptide.

In another embodiment, the recombinantly produced peptide is produced to enhance immune response, for example to increase CAR mediated lysis in cancer cells.

In another embodiment, a therapeutic microbe that expresses a peptide of interest is combined with native microbes designed to enhance the efficacy of the therapy. For example, a microbe expressing a peptide of interest is combined with a Bifidobacterium strain designed to decrease gut permeability and enhance immunological response.

### Engineering of Engraftment Control in Therapeutic Microbes

In one embodiment, human milk oligosaccharides are a common carbon source accessible in the infant gut but rarely found otherwise. This provides a unique opportunity to control engraftment in bacteria that can consume human milk oligosaccharides. This function can be engineered into organisms without the capability to do so, thereby providing a unique environmental niche upon the introduction of human milk oligosaccharides as a prebiotic. The gene loci present in Table 7 are an example, but not an exhaustive set, of human milk oligosaccharide utilization genes that could be used to control engraftment in a genetically modified organism.

TABLE 7
Exemplary human oligosaccharide utilization genes that can be used
in compositions and methods as provided herein (for example, exemplary
genes that can be engineered into organisms, or bacteria, as used
in compositions, mixes and consortia as provided herein):

| Bacteria | Gene Loci |
|---|---|
| Bifidobacerium breve | Bbr_0526 |
| Bifidobacerium breve | Bbr_0527 |
| Bifidobacerium breve | Bbr_0528 |
| Bifidobacerium breve | Bbr_0529 |
| Bifidobacerium breve | Bbr_0530 |
| Bifidobacerium breve | Bbr_1551 |
| Bifidobacerium breve | Bbr_1552 |
| Bifidobacerium breve | Bbr_1553 |
| Bifidobacerium breve | Bbr_1554 |
| Bifidobacerium breve | Bbr_1555 |
| Bifidobacerium breve | Bbr_1556 |
| Bifidobacerium breve | Bbr_1558 |
| Bifidobacerium breve | Bbr_1559 |
| Bifidobacerium breve | Bbr_1560 |
| Bifidobacerium breve | Bbr_1585 |
| Bifidobacerium breve | Bbr_1586 |
| Bifidobacerium breve | Bbr_1587 |
| Bifidobacerium breve | Bbr_1588 |
| Bifidobacerium breve | Bbr_1589 |
| Bifidobacerium breve | Bbr_1590 |
| Bifidobacterium bifidum | BBIF_01022 |
| Bifidobacterium bifidum | BBIF_00180 |
| Bifidobacterium bifidum | BBIF_00172 |
| Bifidobacterium bifidum | BBIF_00503 |
| Bifidobacterium bifidum | BBIF_00503 |
| Bifidobacterium bifidum | BBIF_01401 |
| Bifidobacterium bifidum | BBIF_00527 |
| Bifidobacterium bifidum | BBIF_00528 |
| Bifidobacterium bifidum | BBIF_01068 |
| Bifidobacterium bifidum | BBIF_00546 |
| Bifidobacterium bifidum | BBIF_00547 |
| Bifidobacterium bifidum | BBIF_01155 |
| Bifidobacterium bifidum | BBIF_01261 |
| Bifidobacterium bifidum | BBIF_00173 |
| Bifidobacterium bifidum | BBIF_00174 |
| Bifidobacterium bifidum | BBIF_01067 |
| Bifidobacterium bifidum | BBIF_00178 |
| Bifidobacterium bifidum | BBIF_00179 |
| Bifidobacterium bifidum | BBIF_00177 |
| Bifidobacterium bifidum | BBIF_00369 |
| Bifidobacterium bifidum | BBIF_01218 |
| Bifidobacterium bifidum | BBIF_00871 |
| Bifidobacterium bifidum | BBIF_01021 |
| Bifidobacterium bifidum | BBIF_01217 |
| Bifidobacterium bifidum | BBIF_00368 |
| Bifidobacterium bifidum | BBIF_00533 |
| Bifidobacterium bifidum | BBIF_01442 |
| Bifidobacterium bifidum | BBIF_00707 |
| Bifidobacterium bifidum | BBIF_00008 |
| Bifidobacterium bifidum | BBIF_01050 |
| Bifidobacterium bifidum | BBIF_00852 |
| Bifidobacterium bifidum | BBIF_00176 |
| Bifidobacterium bifidum | BBIF_00136 |
| Bifidobacterium bifidum | BBIF_00175 |
| Bifidobacterium bifidum | BBIF_01445 |
| Bifidobacterium bifidum | BBIF_00550 |
| Bifidobacterium bifidum | BBIF_01073 |
| Bifidobacterium bifidum | BBIF_00706 |
| Bifidobacterium longum | BLNG_00936 |
| Bifidobacterium longum | BLNG_00160 |
| Bifidobacterium longum | BLNG_01262 |
| Bifidobacterium longum | BLNG_01258 |
| Bifidobacterium longum | BLNG_01263 |
| Bifidobacterium longum | BLNG_01259 |
| Bifidobacterium longum | BLNG_01260 |
| Bifidobacterium longum | BLNG_01261 |
| Bifidobacterium longum | BLNG_01264 |
| Bifidobacterium longum | BLNG_01257 |
| Bifidobacterium longum | BLNG_01255 |
| Bifidobacterium longum | BLNG_01256 |
| Bifidobacterium longum | BLNG_00014 |
| Bifidobacterium longum | BLNG_00196 |
| Bifidobacterium longum | BLNG_01135 |
| Bifidobacterium longum | BLNG_01136 |
| Bifidobacterium longum | BLNG_00166 |
| Bifidobacterium longum | BLNG_01475 |
| Bifidobacterium longum | BLNG_01345 |
| Bifidobacterium longum | BLNG_00935 |
| Bifidobacterium longum | BLNG_00933 |
| Bifidobacterium longum | BLNG_00934 |
| Bifidobacterium longum | BLNG_00460 |
| Bifidobacterium longum | BLNG_00163 |
| Bifidobacterium longum | BLNG_00164 |
| Bifidobacterium longum | BLNG_00457 |
| Bifidobacterium longum | BLNG_00458 |
| Bifidobacterium longum | BLNG_00161 |
| Bifidobacterium longum | BLNG_00162 |
| Bifidobacterium longum | BLNG_00911 |
| Bifidobacterium longum | BLNG_00459 |
| Bifidobacterium longum | BLNG_01753 |
| Bifidobacterium pseudocatenulatum | BBPC_RS08970 |
| Bifidobacterium pseudocatenulatum | BBPC_RS08965 |
| Bifidobacterium pseudocatenulatum | BBPC_RS08960 |
| Bifidobacterium pseudocatenulatum | BBPC_RS08955 |
| Bifidobacterium pseudocatenulatum | BBPC_RS08950 |
| Bifidobacterium pseudocatenulatum | BBPC_RS08945 |
| Bifidobacterium pseudocatenulatum | BBPC_RS08940 |
| Bifidobacterium pseudocatenulatum | BBPC_RS08935 |
| Bifidobacterium pseudocatenulatum | Ga0224696_111928 |
| Bifidobacterium pseudocatenulatum | Ga0224696_111927 |
| Bifidobacterium dentium | BBDE_RS03200 |
| Bifidobacterium dentium | BBDE_RS07935 |
| Phocaeicola dorei | GKD17_22625 |
| Phocaeicola dorei | GKD17_19925 |
| Phocaeicola dorei | GKD17_03650 |
| Phocaeicola dorei | GKD17_17105 |
| Phocaeicola dorei | GKD17_15975 |
| Phocaeicola dorei | GKD17_18780 |
| Phocaeicola dorei | GKD17_20055 |
| Phocaeicola dorei | GKD17_17275 |
| Phocaeicola dorei | GKD17_18785 |
| Phocaeicola dorei | GKD17_08535 |
| Phocaeicola dorei | GKD17_05150 |
| Phocaeicola dorei | GKD17_04105 |
| Phocaeicola dorei | GKD17_03925 |
| Phocaeicola dorei | GKD17_04350 |
| Phocaeicola dorei | GKD17_08525 |
| Phocaeicola dorei | GKD17_02615 |
| Phocaeicola dorei | GKD17_15955 |
| Phocaeicola dorei | GKD17_17165 |
| Phocaeicola dorei | GKD17_21680 |
| Phocaeicola dorei | GKD17_19325 |
| Phocaeicola dorei | GKD17_15930 |
| Phocaeicola dorei | GKD17_17900 |
| Phocaeicola dorei | GDK17_14265 |
| Phocaeicola dorei | GKD17_02430 |
| Phocaeicola dorei | GKD17_15970 |
| Phocaeicola dorei | GKD17_15950 |
| Phocaeicola dorei | GKD17_16755 |
| Phocaeicola dorei | GKD17_23170 |
| Phocaeicola dorei | GKD17_15940 |
| Phocaeicola dorei | GKD17_11490 |
| Phocaeicola dorei | GKD17_18995 |
| Phocaeicola dorei | GKD17_04390 |
| Phocaeicola dorei | GKD17_18790 |
| Phocaeicola dorei | GKD17_02610 |
| Phocaeicola dorei | GKD17_18840 |
| Phocaeicola dorei | GKD17_18845 |
| Phocaeicola dorei | GKD17_16495 |
| Phocaeicola dorei | GKD17_19420 |
| Phocaeicola dorei | GKD17_19425 |
| Phocaeicola dorei | GKD17_19440 |
| Phocaeicola dorei | GKD17_15935 |
| Phocaeicola dorei | GKD17_19085 |
| Phocaeicola dorei | GKD17_19390 |
| Phocaeicola dorei | GKD17_21715 |
| Bacteroides thetaiotaomicron | BT3958 |
| Bacteroides thetaiotaomicron | BT3959 |
| Bacteroides thetaiotaomicron | BT3960 |
| Bacteroides thetaiotaomicron | BT3961 |
| Bacteroides thetaiotaomicron | BT3962 |
| Bacteroides thetaiotaomicron | BT3963 |
| Bacteroides thetaiotaomicron | BT3964 |
| Bacteroides thetaiotaomicron | BT3965 |
| Bacteroides thetaiotaomicron | BT2618 |
| Bacteroides thetaiotaomicron | BT2619 |
| Bacteroides thetaiotaomicron | BT2620 |
| Bacteroides thetaiotaomicron | BT2621 |
| Bacteroides thetaiotaomicron | BT2622 |
| Bacteroides thetaiotaomicron | BT2623 |
| Bacteroides thetaiotaomicron | BT2624 |
| Bacteroides thetaiotaomicron | BT2625 |
| Bacteroides thetaiotaomicron | BT2626 |
| Bacteroides thetaiotaomicron | BT2627 |
| Bacteroides thetaiotaomicron | BT2628 |
| Bacteroides thetaiotaomicron | BT2629 |
| Bacteroides thetaiotaomicron | BT2630 |
| Bacteroides thetaiotaomicron | BT2631 |
| Bacteroides thetaiotaomicron | BT2632 |
| Bacteroides thetaiotaomicron | BT2633 |
| Bacteroides thetaiotaomicron | BT3172 |
| Bacteroides thetaiotaomicron | BT3173 |
| Bacteroides thetaiotaomicron | BT3773 |
| Bacteroides thetaiotaomicron | BT3774 |
| Bacteroides thetaiotaomicron | BT3775 |
| Bacteroides thetaiotaomicron | BT3776 |
| Bacteroides thetaiotaomicron | BT3777 |
| Bacteroides thetaiotaomicron | BT3778 |
| Bacteroides thetaiotaomicron | BT3779 |
| Bacteroides thetaiotaomicron | BT3780 |
| Bacteroides thetaiotaomicron | BT3781 |
| Bacteroides thetaiotaomicron | BT3782 |
| Bacteroides thetaiotaomicron | BT3783 |
| Bacteroides thetaiotaomicron | BT3784 |
| Bacteroides thetaiotaomicron | BT3785 |
| Bacteroides thetaiotaomicron | BT3786 |
| Bacteroides thetaiotaomicron | BT3787 |
| Bacteroides thetaiotaomicron | BT3788 |
| Bacteroides thetaiotaomicron | BT3789 |
| Bacteroides thetaiotaomicron | BT3790 |
| Bacteroides thetaiotaomicron | BT3791 |
| Bacteroides thetaiotaomicron | BT3792 |
| Bacteroides thetaiotaomicron | BT4132 |
| Bacteroides thetaiotaomicron | BT4133 |
| Bacteroides thetaiotaomicron | BT4134 |
| Bacteroides thetaiotaomicron | BT4135 |
| Bacteroides thetaiotaomicron | BT4136 |
| Bacteroides thetaiotaomicron | BT0459 |
| Bacteroides thetaiotaomicron | BT0460 |
| Bacteroides thetaiotaomicron | BT0461 |
| Bacteroides thetaiotaomicron | BT1036 |
| Bacteroides thetaiotaomicron | BT1039 |
| Bacteroides thetaiotaomicron | BT1040 |
| Bacteroides thetaiotaomicron | BT1041 |
| Bacteroides thetaiotaomicron | BT1042 |
| Bacteroides thetaiotaomicron | BT1043 |
| Bacteroides thetaiotaomicron | BT1044 |
| Bacteroides thetaiotaomicron | BT1280 |
| Bacteroides thetaiotaomicron | BT1281 |
| Bacteroides thetaiotaomicron | BT1282 |
| Bacteroides thetaiotaomicron | BT1283 |
| Bacteroides thetaiotaomicron | BT1284 |
| Bacteroides thetaiotaomicron | BT1285 |
| Bacteroides thetaiotaomicron | BT1624 |
| Bacteroides thetaiotaomicron | BT1628 |
| Bacteroides thetaiotaomicron | BT1629 |
| Bacteroides thetaiotaomicron | BT1630 |
| Bacteroides thetaiotaomicron | BT1631 |
| Bacteroides thetaiotaomicron | BT1632 |
| Bacteroides thetaiotaomicron | BT2818 |
| Bacteroides thetaiotaomicron | BT2819 |
| Bacteroides thetaiotaomicron | BT2820 |
| Bacteroides thetaiotaomicron | BT2821 |
| Bacteroides thetaiotaomicron | BT2822 |
| Bacteroides thetaiotaomicron | BT2823 |
| Bacteroides thetaiotaomicron | BT2824 |
| Bacteroides thetaiotaomicron | BT2825 |
| Bacteroides thetaiotaomicron | BT3854 |
| Bacteroides thetaiotaomicron | BT3855 |
| Bacteroides thetaiotaomicron | BT3856 |
| Bacteroides thetaiotaomicron | BT3857 |
| Bacteroides thetaiotaomicron | BT3858 |
| Bacteroides thetaiotaomicron | BT3859 |
| Bacteroides thetaiotaomicron | BT3860 |
| Bacteroides thetaiotaomicron | BT3861 |
| Bacteroides thetaiotaomicron | BT3862 |
| Bacteroides thetaiotaomicron | BT4038 |
| Bacteroides thetaiotaomicron | BT4039 |
| Bacteroides thetaiotaomicron | BT4040 |
| Bacteroides thetaiotaomicron | BT4294 |
| Bacteroides thetaiotaomicron | BT4295 |
| Bacteroides thetaiotaomicron | BT4296 |
| Bacteroides thetaiotaomicron | BT4297 |
| Bacteroides thetaiotaomicron | BT4298 |
| Bacteroides thetaiotaomicron | BT4299 |
| Bifidobacterium infantis | Bion_2361 |
| Bifidobacterium infantis | Bion_2360 |
| Bifidobacterium infantis | Bion_2359 |
| Bifidobacterium infantis | Bion_2357 |
| Bifidobacterium infantis | Bion_2355 |
| Bifidobacterium infantis | Bion_2354 |
| Bifidobacterium infantis | Bion_2352 |
| Bifidobacterium infantis | Bion_2351 |
| Bifidobacterium infantis | Bion_2350 |
| Bifidobacterium infantis | Bion_2348 |
| Bifidobacterium infantis | Bion_2347 |
| Bifidobacterium infantis | Bion_2344 |
| Bifidobacterium infantis | Bion_2346 |
| Bifidobacterium infantis | Bion_2343 |
| Bifidobacterium infantis | Bion_2342 |
| Bifidobacterium infantis | Bion_2345 |
| Bifidobacterium infantis | Bion_2336 |
| Bifidobacterium infantis | Bion_2334 |
| Bifidobacterium infantis | Bion_2332 |
| Bifidobacterium infantis | Bion_2331 |
| Bifidobacterium infantis | Bion_0248 |
| Bifidobacterium infantis | Bion_0245 |
| Bifidobacterium infantis | Bion_0244 |
| Bifidobacterium infantis | Bion_0243 |
| Bifidobacterium infantis | Bion_0426 |
| Bifidobacterium infantis | Bion_0423 |
| Bifidobacterium infantis | Bion_0247 |
| Bifidobacterium infantis | Bion_0425 |
| Bifidobacterium infantis | Bion_0645 |
| Bifidobacterium infantis | Bion_0651 |
| Bifidobacterium infantis | Bion_0650 |
| Bifidobacterium infantis | Bion_0648 |
| Bifidobacterium infantis | Bion_0647 |
| Bifidobacterium infantis | Bion_0646 |
| Bifidobacterium infantis | Bion_0644 |
| Bifidobacterium infantis | Bion_0643 |
| Bifidobacterium infantis | Bion_0642 |
| Bifidobacterium infantis | Bion_0641 |
| Bifidobacterium infantis | Bion_0625 |
| Bifidobacterium infantis | Bion_2172 |
| Bifidobacterium infantis | Bion_2177 |
| Bifidobacterium infantis | Bion_2176 |
| Bifidobacterium infantis | Bion_2175 |
| Bifidobacterium infantis | Bion_2174 |
| Bifidobacterium infantis | Bion_2173 |
| Bifidobacterium infantis | Bion_2171 |
| Bifidobacterium infantis | Bion_0114 |
| Bifidobacterium infantis | Bion_0113 |
| Bifidobacterium infantis | BLIJ_0113 |
| Bifidobacterium infantis | Bion_0115 |
| Bifidobacterium infantis | Bion_0111 |
| Bifidobacterium infantis | Bion_0110 |
| Bifidobacterium infantis | Bion_0109 |
| Bifidobacterium infantis | Bion_0108 |
| Bifidobacterium infantis | Bion_0107 |
| Bifidobacterium infantis | Bion_0106 |
| Bifidobacterium infantis | Bion_0105 |
| Bifidobacterium infantis | Bion_0104 |

In another embodiment, rare dietary polysaccharides can be used to control microbial engraftment in the gut. For example, the genes BACPLE_1683-1706 from the Bacteroides plebeius genome (or homologs thereof) are used to consume the polysaccharide porphyran. These genes can be transferred to a new chassis to control engraftment of the new bacteria in the presence of porphyran.

In alternative embodiments, provided are compositions or formulation comprising at least one non-pathogenic, live bacteria and/or non-pathogenic bacterial spore and at least one probiotic (also called a synbiotic, or combination of a probiotic and a prebiotic), for example, as set forth in Table 8:

TABLE 8

| | |
|---|---|
| 1 | Bifidobacterium infantis |
| | 2′-fucosyllactose |
| 2 | Bifidobacterium bifidum |
| | 2′-fucosyllactose |
| 3 | Bifidobacterium infantis |
| | bifidobacterium bifidum |
| | 2′-fucosyllactose |
| 4 | Bifidobacterium infantis |
| | Bifidobacterium longum |
| | 2′-fucosyllactose |
| 5 | Bifidobacterium infantis |
| | Bifidobacterium breve |
| | 2′-fucosyllactose |
| 6 | Bifidobacterium infantis |
| | Bifidobacterium breve |
| | Bifidobacterium longum |
| | 2′-fucosyllactose |
| 7 | Bifidobacterium infantis |
| | Bifidobacterium breve |
| | Bifidobacterium bifidum |
| | 2′-fucosyllactose |
| 8 | Bifidobacterium infantis |
| | Bifidobacterium longum |
| | Bifidobacterium bifidum |
| | 2′-fucosyllactose |
| 9 | Bifidobacterium infantis |
| | Bifidobacterium longum |
| | Bifidobacterium bifidum |
| | Bifidobacterium breve |
| | 2′-fucosyllactose |
| 10 | Bifidobacterium breve |
| | 2′-fucosyllactose |
| 11 | Bifidobacterium longum |
| | 2′-fucosyllactose |
| 12 | Bifidobacterium infantis |
| | Lacto-N-tetraose |
| 13 | Bifidobacterium bifidum |
| | Lacto-N-tetraose |
| 14 | Bifidobacterium infantis |
| | bifidobacterium bifidum |
| | Lacto-N-tetraose |
| 15 | Bifidobacterium infantis |
| | Bifidobacterium longum |
| | Lacto-N-tetraose |
| 16 | Bifidobacterium infantis |
| | Bifidobacterium breve |
| | Lacto-N-tetraose |
| 17 | Bifidobacterium infantis |
| | Bifidobacterium breve |
| | Bifidobacterium longum |
| | Lacto-N-tetraose |
| 18 | Bifidobacterium infantis |
| | Bifidobacterium breve |
| | Bifidobacterium bifidum |
| | Lacto-N-tetraose |
| 19 | Bifidobacterium infantis |
| | Bifidobacterium longum |
| | Bifidobacterium bifidum |
| | Lacto-N-tetraose |
| 20 | Bifidobacterium infantis |
| | Bifidobacterium longum |
| | Bifidobacterium bifidum |
| | Bifidobacterium breve |
| | Lacto-N-tetraose |
| 21 | Bifidobacterium breve |
| | Lacto-N-tetraose |
| 22 | Bifidobacterium longum |
| | Lacto-N-tetraose |
| 33 | Bifidobacterium infantis |
| | 3′-sialyllactose |
| 34 | Bifidobacterium bifidum |
| | 3′-sialyllactose |
| 35 | Bifidobacterium infantis |
| | bifidobacterium bifidum |
| | 3′-sialyllactose |
| 36 | Bifidobacterium infantis |
| | Bifidobacterium longum |
| | 3′-sialyllactose |
| 37 | Bifidobacterium infantis |
| | Bifidobacterium breve |
| | 3′-sialyllactose |
| 38 | Bifidobacterium infantis |
| | Bifidobacterium breve |
| | Bifidobacterium longum |
| | 3′-sialyllactose |
| 39 | Bifidobacterium infantis |
| | Bifidobacterium breve |
| | Bifidobacterium bifidum |
| | 3′-sialyllactose |
| 40 | Bifidobacterium infantis |
| | Bifidobacterium longum |
| | Bifidobacterium bifidum |
| | 3′-sialyllactose |
| 41 | Bifidobacterium infantis |
| | Bifidobacterium longum |
| | Bifidobacterium bifidum |
| | Bifidobacterium breve |
| | 3′-sialyllactose |
| 42 | Bifidobacterium breve |
| | 3′-sialyllactose |
| 43 | Bifidobacterium longum |
| | 3′-sialyllactose |
| 44 | Bifidobacterium infantis |
| | Lacto-N-neotetraose |
| 45 | Bifidobacterium bifidum |
| | Lacto-N-neotetraose |
| 46 | Bifidobacterium infantis |
| | bifidobacterium bifidum |
| | Lacto-N-neotetraose |
| 47 | Bifidobacterium infantis |
| | Bifidobacterium longum |
| | Lacto-N-neotetraose |
| 48 | Bifidobacterium infantis |
| | Bifidobacterium breve |
| | Lacto-N-neotetraose |
| 49 | Bifidobacterium infantis |
| | Bifidobacterium breve |
| | Bifidobacterium longum |
| | Lacto-N-neotetraose |
| 50 | Bifidobacterium infantis |
| | Bifidobacterium breve |
| | Bifidobacterium bifidum |
| | Lacto-N-neotetraose |
| 51 | Bifidobacterium infantis |
| | Bifidobacterium longum |
| | Bifidobacterium bifidum |
| | Lacto-N-neotetraose |
| 52 | Bifidobacterium infantis |
| | Bifidobacterium longum |
| | Bifidobacterium bifidum |
| | Bifidobacterium breve |
| | Lacto-N-neotetraose |
| 53 | Bifidobacterium breve |
| | Lacto-N-neotetraose |
| 54 | Bifidobacterium longum |
| | Lacto-N-neotetraose |
| 55 | Bifidobacterium infantis |
| | 6′-sialyllactose |
| 56 | Bifidobacterium bifidum |
| | 6′-sialyllactose |
| 57 | Bifidobacterium infantis |
| | bifidobacterium bifidum |
| | 6′-sialyllactose |
| 58 | Bifidobacterium infantis |
| | Bifidobacterium longum |
| | 6′-sialyllactose |
| 59 | Bifidobacterium infantis |
| | Bifidobacterium breve |
| | 6′-sialyllactose |
| 60 | Bifidobacterium infantis |
| | Bifidobacterium breve |
| | Bifidobacterium longum |
| | 6′-sialyllactose |
| 61 | Bifidobacterium infantis |
| | Bifidobacterium breve |
| | Bifidobacterium bifidum |
| | 6′-sialyllactose |
| 62 | Bifidobacterium infantis |
| | Bifidobacterium longum |
| | Bifidobacterium bifidum |
| | 6′-sialyllactose |
| 63 | Bifidobacterium infantis |
| | Bifidobacterium longum |
| | Bifidobacterium bifidum |
| | Bifidobacterium breve |
| | 6′-sialyllactose |
| 64 | Bifidobacterium breve |
| | 6′-sialyllactose |
| 65 | Bifidobacterium longum |
| | 6′-sialyllactose |
| 66 | Bifidobacterium infantis |
| | 3-fucosyllactose |
| 67 | Bifidobacterium bifidum |
| | 3-fucosyllactose |
| 68 | Bifidobacterium infantis |
| | bifidobacterium bifidum |
| | 3-fucosyllactose |
| 69 | Bifidobacterium infantis |
| | Bifidobacterium longum |
| | 3-fucosyllactose |
| 70 | Bifidobacterium infantis |
| | Bifidobacterium breve |
| | 3-fucosyllactose |
| 71 | Bifidobacterium infantis |
| | Bifidobacterium breve |
| | Bifidobacterium longum |
| | 3-fucosyllactose |
| 72 | Bifidobacterium infantis |
| | Bifidobacterium breve |
| | Bifidobacterium bifidum |
| | 3-fucosyllactose |
| 73 | Bifidobacterium infantis |
| | Bifidobacterium longum |
| | Bifidobacterium bifidum |
| | 3-fucosyllactose |
| 74 | Bifidobacterium infantis |
| | Bifidobacterium longum |
| | Bifidobacterium bifidum |
| | Bifidobacterium breve |
| | 3-fucosyllactose |
| 75 | Bifidobacterium breve |
| | 3-fucosyllactose |
| 76 | Bifidobacterium longum |
| | 3-fucosyllactose |
| 77 | Bifidobacterium infantis |
| | 2′,3-di-fucosyllactose |
| 78 | Bifidobacterium bifidum |
| | 2′,3-di-fucosyllactose |
| 79 | Bifidobacterium infantis |
| | bifidobacterium bifidum |
| | 2′,3-di-fucosyllactose |
| 80 | Bifidobacterium infantis |
| | Bifidobacterium longum |
| | 2′,3-di-fucosyllactose |
| 81 | Bifidobacterium infantis |
| | Bifidobacterium breve |
| | 2′,3-di-fucosyllactose |
| 82 | Bifidobacterium infantis |
| | Bifidobacterium breve |
| | Bifidobacterium longum |
| | 2′,3-di-fucosyllactose |
| 83 | Bifidobacterium infantis |
| | Bifidobacterium breve |
| | Bifidobacterium bifidum |
| | 2′,3-di-fucosyllactose |
| 84 | Bifidobacterium infantis |
| | Bifidobacterium longum |
| | Bifidobacterium bifidum |
| | 2′,3-di-fucosyllactose |
| 85 | Bifidobacterium infantis |
| | Bifidobacterium longum |
| | Bifidobacterium bifidum |
| | Bifidobacterium breve |
| | 2′,3-di-fucosyllactose |
| 86 | Bifidobacterium breve |
| | 2′,3-di-fucosyllactose |
| 87 | Bifidobacterium longum |
| | 2′,3-di-fucosyllactose |
| 88 | Bifidobacterium infantis |
| | Lacto-N-tetraose |
| | 2′-fucosyllactose |
| | 3′-sialyllactose |
| | 6′-sialyllactose |
| | 2′,3-di-fucosyllactose |
| 89 | Bifidobacterium bifidum |
| | Lacto-N-tetraose |
| | 2′-fucosyllactose |
| | 3′-sialyllactose |
| | 6′-sialyllactose |
| | 2′,3-di-fucosyllactose |
| 90 | Bifidobacterium infantis |
| | bifidobacterium bifidum |
| | Lacto-N-tetraose |
| | 2′-fucosyllactose |
| | 3′-sialyllactose |
| | 6′-sialyllactose |
| | 2′,3-di-fucosyllactose |
| 91 | Bifidobacterium infantis |
| | Bifidobacterium longum |
| | Lacto-N-tetraose |
| | 2′-fucosyllactose |
| | 3′-sialyllactose |
| | 6′-sialyllactose |
| | 2′,3-di-fucosyllactose |
| 92 | Bifidobacterium infantis |
| | Bifidobacterium breve |
| | Lacto-N-tetraose |
| | 2′-fucosyllactose |
| | 3′-sialyllactose |
| | 6′-sialyllactose |
| | 2′,3-di-fucosyllactose |
| 93 | Bifidobacterium infantis |
| | Bifidobacterium breve |
| | Bifidobacterium longum |
| | Lacto-N-tetraose |
| | 2′-fucosyllactose |
| | 3′-sialyllactose |
| | 6′-sialyllactose |
| | 2′,3-di-fucosyllactose |
| 94 | Bifidobacterium infantis |
| | Bifidobacterium breve |
| | Bifidobacterium bifidum |
| | Lacto-N-tetraose |
| | 2′-fucosyllactose |
| | 3′-sialyllactose |
| | 6′-sialyllactose |
| | 2′,3-di-fucosyllactose |
| 95 | Bifidobacterium infantis |
| | Bifidobacterium longum |
| | Bifidobacterium bifidum |
| | Lacto-N-tetraose |
| | 2′-fucosyllactose |
| | 3′-sialyllactose |
| | 6′-sialyllactose |
| | 2′,3-di-fucosyllactose |
| 96 | Bifidobacterium infantis |
| | Bifidobacterium longum |
| | Bifidobacterium bifidum |
| | Bifidobacterium breve |
| | Lacto-N-tetraose |
| | 2′-fucosyllactose |
| | 3′-sialyllactose |
| | 6′-sialyllactose |
| | 2′,3-di-fucosyllactose |
| 97 | Bifidobacterium breve |
| | Lacto-N-tetraose |
| | 2′-fucosyllactose |
| | 3′-sialyllactose |
| | 6′-sialyllactose |
| | 2′,3-di-fucosyllactose |
| 98 | Bifidobacterium longum |
| | Lacto-N-tetraose |
| | 2′-fucosyllactose |
| | 3′-sialyllactose |
| | 6′-sialyllactose |
| | 2′,3-di-fucosyllactose |
| 99 | Bifidobacterium infantis |
| | Lacto-N-tetraose |
| | Lacto-N-neotetraose |
| | 2′-fucosyllactose |
| | 3′-sialyllactose |
| | 6′-sialyllactose |
| 100 | Bifidobacterium bifidum |
| | Lacto-N-tetraose |
| | Lacto-N-neotetraose |
| | 2′-fucosyllactose |
| | 3′-sialyllactose |
| | 6′-sialyllactose |
| 101 | Bifidobacterium infantis |
| | bifidobacterium bifidum |
| | Lacto-N-tetraose |
| | Lacto-N-neotetraose |
| | 2′-fucosyllactose |
| | 3′-sialyllactose |
| | 6′-sialyllactose |
| 102 | Bifidobacterium infantis |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| 103 | Bifidobacterium infantis |
| | Bifidobacterium breve |
| | Lacto-N-tetraose |
| | Lacto-N-neotetraose |
| | 2′-fucosyllactose |
| | 3′-sialyllactose |
| | 6′-sialyllactose |
| 104 | Bifidobacterium infantis |
| | Bifidobacterium breve |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| 105 | Bifidobacterium infantis |
| | Bifidobacterium breve |
| | Bifidobacterium bifidum |
| | Lacto-N-tetraose |
| | Lacto-N-neotetraose |
| | 2′-fucosyllactose |
| | 3′-sialyllactose |
| | 6′-sialyllactose |
| 106 | Bifidobacterium infantis |
| | Bifidobacterium longum |
| | Bifidobacterium bifidum |
| | Lacto-N-tetraose |
| | Lacto-N-neotetraose |
| | 2′-fucosyllactose |
| | 3′-sialyllactose |
| | 6′-sialyllactose |
| 107 | Bifidobacterium infantis |
| | Bifidobacterium longum |
| | Bifidobacterium bifidum |
| | Bifidobacterium breve |
| | Lacto-N-tetraose |
| | Lacto-N-neotetraose |
| | 2′-fucosyllactose |
| | 3′-sialyllactose |
| | 6′-sialyllactose |
| 108 | Bifidobacterium breve |
| | Lacto-N-tetraose |
| | Lacto-N-neotetraose |
| | 2′-fucosyllactose |
| | 3′-sialyllactose |
| | 6′-sialyllactose |
| 109 | Bifidobacterium longum |
| | Lacto-N-tetraose |
| | Lacto-N-neotetraose |
| | 2′-fucosyllactose |
| | 3′-sialyllactose |
| | 6′-sialyllactose |
| 110 | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 111 | Bifidobacterium bifidum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 112 | Bifidobacterium infantis |
| | bifidobacterium bifidum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 113 | Bifidobacterium infantis |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 114 | Bifidobacterium infantis |
| | Bifidobacterium breve |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 115 | Bifidobacterium infantis |
| | Bifidobacterium breve |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 116 | Bifidobacterium infantis |
| | Bifidobacterium breve |
| | Bifidobacterium bifidum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 117 | Bifidobacterium infantis |
| | Bifidobacterium longum |
| | Bifidobacterium bifidum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 118 | Bifidobacterium infantis |
| | Bifidobacterium longum |
| | Bifidobacterium bifidum |
| | Bifidobacterium breve |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 119 | Bifidobacterium breve |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 120 | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 121 | Bacteroides thetaiotaomicron |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 122 | Bacteroides thetaiotaomicron |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 123 | Bacteroides thetaiotaomicron |
| | Lacto-N-tetraose |
| | 2′-fucosyllactose |
| | 3′-sialyllactose |
| | 6′-sialyllactose |
| | 2′,3-di-fucosyllactose |
| 124 | Bacteroides thetaiotaomicron |
| | Lacto-N-tetraose |
| 125 | Bacteroides thetaiotaomicron |
| | Lacto-N-neotetraose |
| 126 | Bacteroides thetaiotaomicron |
| | 2′-fucosyllactose |
| 127 | Bacteroides thetaiotaomicron |
| | 3′-sialyllactose |
| 128 | Bacteroides thetaiotaomicron |
| | 6′-sialyllactose |
| 129 | Bacteroides thetaiotaomicron |
| | 3-fucosyllactose |
| 130 | Bifidobacterium infantis |
| | Mucin |
| 131 | Bifidobacterium bifidum |
| | Mucin |
| 132 | Bifidobacterium infantis |
| | bifidobacterium bifidum |
| | Mucin |
| 133 | Bifidobacterium infantis |
| | Bifidobacterium longum |
| | Mucin |
| 134 | Bifidobacterium infantis |
| | Bifidobacterium breve |
| | Mucin |
| 135 | Bifidobacterium infantis |
| | Bifidobacterium breve |
| | Bifidobacterium longum |
| | Mucin |
| 136 | Bifidobacterium infantis |
| | Bifidobacterium breve |
| | Bifidobacterium bifidum |
| | Mucin |
| 137 | Bifidobacterium infantis |
| | Bifidobacterium longum |
| | Bifidobacterium bifidum |
| | Mucin |
| 138 | Bifidobacterium infantis |
| | Bifidobacterium longum |
| | Bifidobacterium bifidum |
| | Bifidobacterium breve |
| | Mucin |
| 139 | Bifidobacterium breve |
| | Mucin |
| 140 | Bifidobacterium longum |
| | Mucin |
| 141 | Bifidobacterium bifidum |
| | 2′-fucosyllactose |
| | mucin |
| 142 | Bifidobacterium infantis |
| | bifidobacterium bifidum |
| | 2′-fucosyllactose |
| | mucin |
| 143 | Bifidobacterium infantis |
| | Bifidobacterium longum |
| | 2′-fucosyllactose |
| | mucin |
| 144 | Bifidobacterium infantis |
| | Bifidobacterium breve |
| | 2′-fucosyllactose |
| | mucin |
| 145 | Bifidobacterium infantis |
| | Bifidobacterium breve |
| | Bifidobacterium longum |
| | 2′-fucosyllactose |
| | mucin |
| 146 | Bifidobacterium infantis |
| | Bifidobacterium breve |
| | Bifidobacterium bifidum |
| | 2′-fucosyllactose |
| | mucin |
| 147 | Bifidobacterium infantis |
| | Bifidobacterium longum |
| | Bifidobacterium bifidum |
| | 2′-fucosyllactose |
| | mucin |
| 148 | Bifidobacterium infantis |
| | Bifidobacterium longum |
| | Bifidobacterium bifidum |
| | Bifidobacterium breve |
| | 2′-fucosyllactose |
| | mucin |
| 149 | Bifidobacterium breve |
| | 2′-fucosyllactose |
| | mucin |
| 150 | Bifidobacterium longum |
| | 2′-fucosyllactose |
| | mucin |
| 151 | Bifidobacterium bifidum |
| | 2′-fucosyllactose |
| | Ellagic Acid |
| | Ellagitanin |
| 152 | Bifidobacterium infantis |
| | bifidobacterium bifidum |
| | 2′-fucosyllactose |
| | Ellagic Acid |
| | Ellagitanin |
| 153 | Bifidobacterium infantis |
| | Bifidobacterium longum |
| | 2′-fucosyllactose |
| | Ellagic Acid |
| | Ellagitanin |
| 154 | Bifidobacterium infantis |
| | Bifidobacterium breve |
| | 2′-fucosyllactose |
| | Ellagic Acid |
| | Ellagitanin |
| 155 | Bifidobacterium infantis |
| | Bifidobacterium breve |
| | Bifidobacterium longum |
| | 2′-fucosyllactose |
| | Ellagic Acid |
| | Ellagitanin |
| 156 | Bifidobacterium infantis |
| | Bifidobacterium breve |
| | Bifidobacterium bifidum |
| | 2′-fucosyllactose |
| | Ellagic Acid |
| | Ellagitanin |
| 157 | Bifidobacterium infantis |
| | Bifidobacterium longum |
| | Bifidobacterium bifidum |
| | 2′-fucosyllactose |
| | Ellagic Acid |
| | Ellagitanin |
| 158 | Bifidobacterium infantis |
| | Bifidobacterium longum |
| | Bifidobacterium bifidum |
| | Bifidobacterium breve |
| | 2′-fucosyllactose |
| | Ellagic Acid |
| | Ellagitanin |
| 159 | Bifidobacterium breve |
| | 2′-fucosyllactose |
| | Ellagic Acid |
| | Ellagitanin |
| 160 | Bifidobacterium longum |
| | 2′-fucosyllactose |
| | Ellagic Acid |
| | Ellagitanin |
| 161 | Bifidobacterium infantis |
| | 2′-fucosyllactose |
| | mucin |
| 162 | Bifidobacterium infantis |
| | 2′-fucosyllactose |
| | Ellagic Acid |
| | Ellagitanin |

In alternative embodiments, provided are compositions or formulation comprising at least one non-pathogenic, live bacteria and/or non-pathogenic bacterial spore and at least one probiotic (also called a synbiotic, or combination of a probiotic and a prebiotic) with a strain specific designation, for example, as set forth in Table 32:

TABLE 32

| | |
|---|---|
| 1 | PB-STR-093 |
| | 2′-fucosyllactose |
| 2 | PB-STR-207 |
| | 2′-fucosyllactose |
| 3 | PB-STR-083 |
| | 2′-fucosyllactose |
| 4 | PB-STR-119 |
| | 2′-fucosyllactose |
| 5 | PB-STR-093 |
| | Bifidobacterium infantis |
| | 2′-fucosyllactose |
| 6 | PB-STR-093 |
| | Bifidobacterium longum |
| | 2′-fucosyllactose |
| 7 | PB-STR-093 |
| | Bifidobacterium breve |
| | 2′-fucosyllactose |
| 8 | PB-STR-207 |
| | Bifidobacterium infantis |
| | 2′-fucosyllactose |
| 9 | PB-STR-207 |
| | Bifidobacterium longum |
| | 2′-fucosyllactose |
| 10 | PB-STR-207 |
| | Bifidobacterium breve |
| | 2′-fucosyllactose |
| 11 | PB-STR-207 |
| | Bifidobacterium bifidum |
| | 2′-fucosyllactose |
| 12 | PB-STR-083 |
| | Bifidobacterium infantis |
| | 2′-fucosyllactose |
| 13 | PB-STR-083 |
| | Bifidobacterium longum |
| | 2′-fucosyllactose |
| 14 | PB-STR-083 |
| | Bifidobacterium breve |
| | 2′-fucosyllactose |
| 15 | PB-STR-119 |
| | Bifidobacterium infantis |
| | 2′-fucosyllactose |
| 16 | PB-STR-119 |
| | Bifidobacterium longum |
| | 2′-fucosyllactose |
| 17 | PB-STR-119 |
| | Bifidobacterium breve |
| | 2′-fucosyllactose |
| 18 | PB-STR-093 |
| | Bifidobacterium longum |
| | Bifidobacterium infantis |
| | 2′-fucosyllactose |
| 19 | PB-STR-093 |
| | Bifidobacterium breve |
| | Bifidobacterium infantis |
| | 2′-fucosyllactose |
| 20 | PB-STR-093 |
| | Bifidobacterium breve |
| | Bifidobacterium longum |
| | 2′-fucosyllactose |
| 21 | PB-STR-207 |
| | Bifidobacterium longum |
| | Bifidobacterium infantis |
| | 2′-fucosyllactose |
| 22 | PB-STR-207 |
| | Bifidobacterium breve |
| | Bifidobacterium infantis |
| | 2′-fucosyllactose |
| 23 | PB-STR-207 |
| | Bifidobacterium breve |
| | Bifidobacterium longum |
| | 2′-fucosyllactose |
| 24 | PB-STR-119 |
| | Bifidobacterium longum |
| | Bifidobacterium infantis |
| | 2′-fucosyllactose |
| | PB-STR-119 |
| 25 | Bifidobacterium breve |
| | Bifidobacterium infantis |
| | 2′-fucosyllactose |
| 26 | PB-STR-119 |
| | Bifidobacterium breve |
| | Bifidobacterium longum |
| | 2′-fucosyllactose |
| 27 | PB-STR-083 |
| | Bifidobacterium longum |
| | Bifidobacterium infantis |
| | 2′-fucosyllactose |
| 28 | PB-STR-083 |
| | Bifidobacterium breve |
| | Bifidobacterium infantis |
| | 2′-fucosyllactose |
| 29 | PB-STR-083 |
| | Bifidobacterium breve |
| | Bifidobacterium longum |
| | 2′-fucosyllactose |
| 30 | PB-STR-093 |
| | Bifidobacterium longum |
| | Bifidobacterium breve |
| | Bifidobacterium infantis |
| | 2′-fucosyllactose |
| 31 | PB-STR-207 |
| | Bifidobacterium longum |
| | Bifidobacterium breve |
| | Bifidobacterium infantis |
| | 2′-fucosyllactose |
| 32 | PB-STR-119 |
| | Bifidobacterium longum |
| | Bifidobacterium breve |
| | Bifidobacterium infantis |
| | 2′-fucosyllactose |
| 33 | PB-STR-083 |
| | Bifidobacterium longum |
| | Bifidobacterium breve |
| | Bifidobacterium infantis |
| | 2′-fucosyllactose |
| 34 | PB-STR-093 |
| | PB-STR-207 |
| | PB-STR-119 |
| | PB-STR-083 |
| | 2′-fucosyllactose |
| 35 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 2′-fucosyllactose |
| 36 | PB-STR-207 |
| | PB-STR-215 |
| | PB-STR-220 |
| | 2′-fucosyllactose |
| 37 | PB-STR-003 |
| | PB-STR-093 |
| | PB-STR-034 |
| | PB-STR-035 |
| | PB-STR-083 |
| | 2′-fucosyllactose |
| 38 | PB-STR-119 |
| | PB-STR-103 |
| | 2′-fucosyllactose |
| 39 | PB-STR-093 |
| | Lacto-N-tetraose |
| 40 | PB-STR-207 |
| | Lacto-N-tetraose |
| 41 | PB-STR-083 |
| | Lacto-N-tetraose |
| 42 | PB-STR-119 |
| | Lacto-N-tetraose |
| 43 | PB-STR-093 |
| | Bifidobacterium infantis |
| | Lacto-N-tetraose |
| 44 | PB-STR-093 |
| | Bifidobacterium longum |
| | Lacto-N-tetraose |
| 45 | PB-STR-093 |
| | Bifidobacterium breve |
| | Lacto-N-tetraose |
| 46 | PB-STR-207 |
| | Bifidobacterium infantis |
| | Lacto-N-tetraose |
| 47 | PB-STR-207 |
| | Bifidobacterium longum |
| | Lacto-N-tetraose |
| 48 | PB-STR-207 |
| | Bifidobacterium breve |
| | Lacto-N-tetraose |
| 49 | PB-STR-207 |
| | Bifidobacterium bifidum |
| | Lacto-N-tetraose |
| 50 | PB-STR-083 |
| | Bifidobacterium infantis |
| | Lacto-N-tetraose |
| 51 | PB-STR-083 |
| | Bifidobacterium longum |
| | Lacto-N-tetraose |
| 52 | PB-STR-083 |
| | Bifidobacterium breve |
| | Lacto-N-tetraose |
| 53 | PB-STR-119 |
| | Bifidobacterium infantis |
| | Lacto-N-tetraose |
| 54 | PB-STR-119 |
| | Bifidobacterium longum |
| | Lacto-N-tetraose |
| 55 | PB-STR-119 |
| | Bifidobacterium breve |
| | Lacto-N-tetraose |
| 56 | PB-STR-093 |
| | Bifidobacterium longum |
| | Bifidobacterium infantis |
| | Lacto-N-tetraose |
| 57 | PB-STR-093 |
| | Bifidobacterium breve |
| | Bifidobacterium infantis |
| | Lacto-N-tetraose |
| 58 | PB-STR-093 |
| | Bifidobacterium breve |
| | Bifidobacterium longum |
| | Lacto-N-tetraose |
| 59 | PB-STR-207 |
| | Bifidobacterium longum |
| | Bifidobacterium infantis |
| | Lacto-N-tetraose |
| 60 | PB-STR-207 |
| | Bifidobacterium breve |
| | Bifidobacterium infantis |
| | Lacto-N-tetraose |
| 61 | PB-STR-207 |
| | Bifidobacterium breve |
| | Bifidobacterium longum |
| | Lacto-N-tetraose |
| 62 | PB-STR-119 |
| | Bifidobacterium longum |
| | Bifidobacterium infantis |
| | Lacto-N-tetraose |
| 63 | PB-STR-119 |
| | Bifidobacterium breve |
| | Bifidobacterium infantis |
| | Lacto-N-tetraose |
| 64 | PB-STR-119 |
| | Bifidobacterium breve |
| | Bifidobacterium longum |
| | Lacto-N-tetraose |
| 65 | PB-STR-083 |
| | Bifidobacterium longum |
| | Bifidobacterium infantis |
| | Lacto-N-tetraose |
| 66 | PB-STR-083 |
| | Bifidobacterium breve |
| | Bifidobacterium infantis |
| | Lacto-N-tetraose |
| 67 | PB-STR-083 |
| | Bifidobacterium breve |
| | Bifidobacterium longum |
| | Lacto-N-tetraose |
| 68 | PB-STR-093 |
| | Bifidobacterium longum |
| | Bifidobacterium breve |
| | Bifidobacterium infantis |
| | Lacto-N-tetraose |
| 69 | PB-STR-207 |
| | Bifidobacterium longum |
| | Bifidobacterium breve |
| | Bifidobacterium infantis |
| | Lacto-N-tetraose |
| 70 | PB-STR-119 |
| | Bifidobacterium longum |
| | Bifidobacterium breve |
| | Bifidobacterium infantis |
| | Lacto-N-tetraose |
| 71 | PB-STR-083 |
| | Bifidobacterium longum |
| | Bifidobacterium breve |
| | Bifidobacterium infantis |
| | Lacto-N-tetraose |
| 72 | PB-STR-093 |
| | PB-STR-207 |
| | PB-STR-119 |
| | PB-STR-083 |
| | Lacto-N-tetraose |
| 73 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | Lacto-N-tetraose |
| 74 | PB-STR-207 |
| | PB-STR-215 |
| | PB-STR-220 |
| | Lacto-N-tetraose |
| 75 | PB-STR-003 |
| | PB-STR-093 |
| | PB-STR-034 |
| | PB-STR-035 |
| | PB-STR-083 |
| | Lacto-N-tetraose |
| 76 | PB-STR-119 |
| | PB-STR-103 |
| | Lacto-N-tetraose |
| 77 | PB-STR-093 |
| | Lacto-N-neotetraose |
| 78 | PB-STR-207 |
| | Lacto-N-neotetraose |
| 79 | PB-STR-083 |
| | Lacto-N-neotetraose |
| 80 | PB-STR-119 |
| | Lacto-N-neotetraose |
| 81 | PB-STR-093 |
| | Bifidobacterium infantis |
| | Lacto-N-neotetraose |
| 82 | PB-STR-093 |
| | Bifidobacterium longum |
| | Lacto-N-neotetraose |
| 83 | PB-STR-093 |
| | Bifidobacterium breve |
| | Lacto-N-neotetraose |
| 84 | PB-STR-207 |
| | Bifidobacterium infantis |
| | Lacto-N-neotetraose |
| 85 | PB-STR-207 |
| | Bifidobacterium longum |
| | Lacto-N-neotetraose |
| 86 | PB-STR-207 |
| | Bifidobacterium breve |
| | Lacto-N-neotetraose |
| 87 | PB-STR-207 |
| | Bifidobacterium bifidum |
| | Lacto-N-neotetraose |
| 88 | PB-STR-083 |
| | Bifidobacterium infantis |
| | Lacto-N-neotetraose |
| 89 | PB-STR-083 |
| | Bifidobacterium longum |
| | Lacto-N-neotetraose |
| 90 | PB-STR-083 |
| | Bifidobacterium breve |
| | Lacto-N-neotetraose |
| 91 | PB-STR-119 |
| | Bifidobacterium infantis |
| | Lacto-N-neotetraose |
| 92 | PB-STR-119 |
| | Bifidobacterium longum |
| | Lacto-N-neotetraose |
| 93 | PB-STR-119 |
| | Bifidobacterium breve |
| | Lacto-N-neotetraose |
| 94 | PB-STR-093 |
| | Bifidobacterium longum |
| | Bifidobacterium infantis |
| | Lacto-N-neotetraose |
| 95 | PB-STR-093 |
| | Bifidobacterium breve |
| | Bifidobacterium infantis |
| | Lacto-N-neotetraose |
| 96 | PB-STR-093 |
| | Bifidobacterium breve |
| | Bifidobacterium longum |
| | Lacto-N-neotetraose |
| 97 | PB-STR-207 |
| | Bifidobacterium longum |
| | Bifidobacterium infantis |
| | Lacto-N-neotetraose |
| 98 | PB-STR-207 |
| | Bifidobacterium breve |
| | Bifidobacterium infantis |
| | Lacto-N-neotetraose |
| 99 | PB-STR-207 |
| | Bifidobacterium breve |
| | Bifidobacterium longum |
| | Lacto-N-neotetraose |
| 100 | PB-STR-119 |
| | Bifidobacterium longum |
| | Bifidobacterium infantis |
| | Lacto-N-neotetraose |
| 101 | PB-STR-119 |
| | Bifidobacterium breve |
| | Bifidobacterium infantis |
| | Lacto-N-neotetraose |
| 102 | PB-STR-119 |
| | Bifidobacterium breve |
| | Bifidobacterium longum |
| | Lacto-N-neotetraose |
| 103 | PB-STR-083 |
| | Bifidobacterium longum |
| | Bifidobacterium infantis |
| | Lacto-N-neotetraose |
| 104 | PB-STR-083 |
| | Bifidobacterium breve |
| | Bifidobacterium infantis |
| | Lacto-N-neotetraose |
| 105 | PB-STR-083 |
| | Bifidobacterium breve |
| | Bifidobacterium longum |
| | Lacto-N-neotetraose |
| 106 | PB-STR-093 |
| | Bifidobacterium longum |
| | Bifidobacterium breve |
| | Bifidobacterium infantis |
| | Lacto-N-neotetraose |
| 107 | PB-STR-207 |
| | Bifidobacterium longum |
| | Bifidobacterium breve |
| | Bifidobacterium infantis |
| | Lacto-N-neotetraose |
| 108 | PB-STR-119 |
| | Bifidobacterium longum |
| | Bifidobacterium breve |
| | Bifidobacterium infantis |
| | Lacto-N-neotetraose |
| 109 | PB-STR-083 |
| | Bifidobacterium longum |
| | Bifidobacterium breve |
| | Bifidobacterium infantis |
| | Lacto-N-neotetraose |
| 110 | PB-STR-093 |
| | PB-STR-207 |
| | PB-STR-119 |
| | PB-STR-083 |
| | Lacto-N-neotetraose |
| 111 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | Lacto-N-neotetraose |
| 112 | PB-STR-207 |
| | PB-STR-215 |
| | PB-STR-220 |
| | Lacto-N-neotetraose |
| 113 | PB-STR-003 |
| | PB-STR-093 |
| | PB-STR-034 |
| | PB-STR-035 |
| | PB-STR-083 |
| | Lacto-N-neotetraose |
| 114 | PB-STR-119 |
| | PB-STR-103 |
| | Lacto-N-neotetraose |
| 115 | PB-STR-093 |
| | 3′-sialyllactose |
| 116 | PB-STR-207 |
| | 3′-sialyllactose |
| 117 | PB-STR-083 |
| | 3′-sialyllactose |
| 118 | PB-STR-119 |
| | 3′-sialyllactose |
| 119 | PB-STR-093 |
| | Bifidobacterium infantis |
| | 3′-sialyllactose |
| 120 | PB-STR-093 |
| | Bifidobacterium longum |
| | 3′-sialyllactose |
| 121 | PB-STR-093 |
| | Bifidobacterium breve |
| | 3′-sialyllactose |
| 122 | PB-STR-207 |
| | Bifidobacterium infantis |
| | 3′-sialyllactose |
| 123 | PB-STR-207 |
| | Bifidobacterium longum |
| | 3′-sialyllactose |
| 124 | PB-STR-207 |
| | Bifidobacterium breve |
| | 3′-sialyllactose |
| 125 | PB-STR-207 |
| | Bifidobacterium bifidum |
| | 3′-sialyllactose |
| 126 | PB-STR-083 |
| | Bifidobacterium infantis |
| | 3′-sialyllactose |
| 127 | PB-STR-083 |
| | Bifidobacterium longum |
| | 3′-sialyllactose |
| 128 | PB-STR-083 |
| | Bifidobacterium breve |
| | 3′-sialyllactose |
| 129 | PB-STR-119 |
| | Bifidobacterium infantis |
| | 3′-sialyllactose |
| 130 | PB-STR-119 |
| | Bifidobacterium longum |
| | 3′-sialyllactose |
| 131 | PB-STR-119 |
| | Bifidobacterium breve |
| | 3′-sialyllactose |
| 132 | PB-STR-093 |
| | Bifidobacterium longum |
| | Bifidobacterium infantis |
| | 3′-sialyllactose |
| 133 | PB-STR-093 |
| | Bifidobacterium breve |
| | Bifidobacterium infantis |
| | 3′-sialyllactose |
| 134 | PB-STR-093 |
| | Bifidobacterium breve |
| | Bifidobacterium longum |
| | 3′-sialyllactose |
| 135 | PB-STR-207 |
| | Bifidobacterium longum |
| | Bifidobacterium infantis |
| | 3′-sialyllactose |
| 136 | PB-STR-207 |
| | Bifidobacterium breve |
| | Bifidobacterium infantis |
| | 3′-sialyllactose |
| 137 | PB-STR-207 |
| | Bifidobacterium breve |
| | Bifidobacterium longum |
| | 3′-sialyllactose |
| 138 | PB-STR-119 |
| | Bifidobacterium longum |
| | Bifidobacterium infantis |
| | 3′-sialyllactose |
| 139 | PB-STR-119 |
| | Bifidobacterium breve |
| | Bifidobacterium infantis |
| | 3′-sialyllactose |
| 140 | PB-STR-119 |
| | Bifidobacterium breve |
| | Bifidobacterium longum |
| | 3′-sialyllactose |
| 141 | PB-STR-083 |
| | Bifidobacterium longum |
| | Bifidobacterium infantis |
| | 3′-sialyllactose |
| 142 | PB-STR-083 |
| | Bifidobacterium breve |
| | Bifidobacterium infantis |
| | 3′-sialyllactose |
| 143 | PB-STR-083 |
| | Bifidobacterium breve |
| | Bifidobacterium longum |
| | 3′-sialyllactose |
| 144 | PB-STR-093 |
| | Bifidobacterium longum |
| | Bifidobacterium breve |
| | Bifidobacterium infantis |
| | 3′-sialyllactose |
| 145 | PB-STR-207 |
| | Bifidobacterium longum |
| | Bifidobacterium breve |
| | Bifidobacterium infantis |
| | 3′-sialyllactose |
| 146 | PB-STR-119 |
| | Bifidobacterium longum |
| | Bifidobacterium breve |
| | Bifidobacterium infantis |
| | 3′-sialyllactose |
| 147 | PB-STR-083 |
| | Bifidobacterium longum |
| | Bifidobacterium breve |
| | Bifidobacterium infantis |
| | 3′-sialyllactose |
| 148 | PB-STR-093 |
| | PB-STR-207 |
| | PB-STR-119 |
| | PB-STR-083 |
| | 3′-sialyllactose |
| 149 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 3′-sialyllactose |
| 150 | PB-STR-207 |
| | PB-STR-215 |
| | PB-STR-220 |
| | 3′-sialyllactose |
| 151 | PB-STR-003 |
| | PB-STR-093 |
| | PB-STR-034 |
| | PB-STR-035 |
| | PB-STR-083 |
| | 3′-sialyllactose |
| 152 | PB-STR-119 |
| | PB-STR-103 |
| | 3′-sialyllactose |
| 153 | PB-STR-093 |
| | 6′-sialyllactose |
| 154 | PB-STR-207 |
| | 6′-sialyllactose |
| 155 | PB-STR-083 |
| | 6′-sialyllactose |
| 156 | PB-STR-119 |
| | 6′-sialyllactose |
| 157 | PB-STR-093 |
| | Bifidobacterium infantis |
| | 6′-sialyllactose |
| 158 | PB-STR-093 |
| | Bifidobacterium longum |
| | 6′-sialyllactose |
| 159 | PB-STR-093 |
| | Bifidobacterium breve |
| | 6′-sialyllactose |
| 160 | PB-STR-207 |
| | Bifidobacterium infantis |
| | 6′-sialyllactose |
| 161 | PB-STR-207 |
| | Bifidobacterium longum |
| | 6′-sialyllactose |
| 162 | PB-STR-207 |
| | Bifidobacterium breve |
| | 6′-sialyllactose |
| 163 | PB-STR-207 |
| | Bifidobacterium bifidum |
| | 6′-sialyllactose |
| 164 | PB-STR-083 |
| | Bifidobacterium infantis |
| | 6′-sialyllactose |
| 165 | PB-STR-083 |
| | Bifidobacterium longum |
| | 6′-sialyllactose |
| 166 | PB-STR-083 |
| | Bifidobacterium breve |
| | 6′-sialyllactose |
| 167 | PB-STR-119 |
| | Bifidobacterium infantis |
| | 6′-sialyllactose |
| 168 | PB-STR-119 |
| | Bifidobacterium longum |
| | 6′-sialyllactose |
| 169 | PB-STR-119 |
| | Bifidobacterium breve |
| | 6′-sialyllactose |
| 170 | PB-STR-093 |
| | Bifidobacterium longum |
| | Bifidobacterium infantis |
| | 6′-sialyllactose |
| 171 | PB-STR-093 |
| | Bifidobacterium breve |
| | Bifidobacterium infantis |
| | 6′-sialyllactose |
| 172 | PB-STR-093 |
| | Bifidobacterium breve |
| | Bifidobacterium longum |
| | 6′-sialyllactose |
| 173 | PB-STR-207 |
| | Bifidobacterium longum |
| | Bifidobacterium infantis |
| | 6′-sialyllactose |
| 174 | PB-STR-207 |
| | Bifidobacterium breve |
| | Bifidobacterium infantis |
| | 6′-sialyllactose |
| 175 | PB-STR-207 |
| | Bifidobacterium breve |
| | Bifidobacterium longum |
| | 6′-sialyllactose |
| 176 | PB-STR-119 |
| | Bifidobacterium longum |
| | Bifidobacterium infantis |
| | 6′-sialyllactose |
| 177 | PB-STR-119 |
| | Bifidobacterium breve |
| | Bifidobacterium infantis |
| | 6′-sialyllactose |
| 178 | PB-STR-119 |
| | Bifidobacterium breve |
| | Bifidobacterium longum |
| | 6′-sialyllactose |
| 179 | PB-STR-083 |
| | Bifidobacterium longum |
| | Bifidobacterium infantis |
| | 6′-sialyllactose |
| 180 | PB-STR-083 |
| | Bifidobacterium breve |
| | Bifidobacterium infantis |
| | 6′-sialyllactose |
| 181 | PB-STR-083 |
| | Bifidobacterium breve |
| | Bifidobacterium longum |
| | 6′-sialyllactose |
| 182 | PB-STR-093 |
| | Bifidobacterium longum |
| | Bifidobacterium breve |
| | Bifidobacterium infantis |
| | 6′-sialyllactose |
| 183 | PB-STR-207 |
| | Bifidobacterium longum |
| | Bifidobacterium breve |
| | Bifidobacterium infantis |
| | 6′-sialyllactose |
| 184 | PB-STR-119 |
| | Bifidobacterium longum |
| | Bifidobacterium breve |
| | Bifidobacterium infantis |
| | 6′-sialyllactose |
| 185 | PB-STR-083 |
| | Bifidobacterium longum |
| | Bifidobacterium breve |
| | Bifidobacterium infantis |
| | 6′-sialyllactose |
| 186 | PB-STR-093 |
| | PB-STR-207 |
| | PB-STR-119 |
| | PB-STR-083 |
| | 6′-sialyllactose |
| 187 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 6′-sialyllactose |
| 188 | PB-STR-207 |
| | PB-STR-215 |
| | PB-STR-220 |
| | 6′-sialyllactose |
| 189 | PB-STR-003 |
| | PB-STR-093 |
| | PB-STR-034 |
| | PB-STR-035 |
| | PB-STR-083 |
| | 6′-sialyllactose |
| 190 | PB-STR-119 |
| | PB-STR-103 |
| | 6′-sialyllactose |
| 191 | PB-STR-093 |
| | 3-fucosyllactose |
| 192 | PB-STR-207 |
| | 3-fucosyllactose |
| 193 | PB-STR-083 |
| | 3-fucosyllactose |
| 194 | PB-STR-119 |
| | 3-fucosyllactose |
| 195 | PB-STR-093 |
| | Bifidobacterium infantis |
| | 3-fucosyllactose |
| 196 | PB-STR-093 |
| | Bifidobacterium longum |
| | 3-fucosyllactose |
| 197 | PB-STR-093 |
| | Bifidobacterium breve |
| | 3-fucosyllactose |
| 198 | PB-STR-207 |
| | Bifidobacterium infantis |
| | 3-fucosyllactose |
| 199 | PB-STR-207 |
| | Bifidobacterium longum |
| | 3-fucosyllactose |
| 200 | PB-STR-207 |
| | Bifidobacterium breve |
| | 3-fucosyllactose |
| 201 | PB-STR-207 |
| | Bifidobacterium bifidum |
| | 3-fucosyllactose |
| 202 | PB-STR-083 |
| | Bifidobacterium infantis |
| | 3-fucosyllactose |
| 203 | PB-STR-083 |
| | Bifidobacterium longum |
| | 3-fucosyllactose |
| 204 | PB-STR-083 |
| | Bifidobacterium breve |
| | 3-fucosyllactose |
| 205 | PB-STR-119 |
| | Bifidobacterium infantis |
| | 3-fucosyllactose |
| 206 | PB-STR-119 |
| | Bifidobacterium longum |
| | 3-fucosyllactose |
| 207 | PB-STR-119 |
| | Bifidobacterium breve |
| | 3-fucosyllactose |
| 208 | PB-STR-093 |
| | Bifidobacterium longum |
| | Bifidobacterium infantis |
| | 3-fucosyllactose |
| 209 | PB-STR-093 |
| | Bifidobacterium breve |
| | Bifidobacterium infantis |
| | 3-fucosyllactose |
| 210 | PB-STR-093 |
| | Bifidobacterium breve |
| | Bifidobacterium longum |
| | 3-fucosyllactose |
| 211 | PB-STR-207 |
| | Bifidobacterium longum |
| | Bifidobacterium infantis |
| | 3-fucosyllactose |
| 212 | PB-STR-207 |
| | Bifidobacterium breve |
| | Bifidobacterium infantis |
| | 3-fucosyllactose |
| 213 | PB-STR-207 |
| | Bifidobacterium breve |
| | Bifidobacterium longum |
| | 3-fucosyllactose |
| 214 | PB-STR-119 |
| | Bifidobacterium longum |
| | Bifidobacterium infantis |
| | 3-fucosyllactose |
| 215 | PB-STR-119 |
| | Bifidobacterium breve |
| | Bifidobacterium infantis |
| | 3-fucosyllactose |
| 216 | PB-STR-119 |
| | Bifidobacterium breve |
| | Bifidobacterium longum |
| | 3-fucosyllactose |
| 217 | PB-STR-083 |
| | Bifidobacterium longum |
| | Bifidobacterium infantis |
| | 3-fucosyllactose |
| 218 | PB-STR-083 |
| | Bifidobacterium breve |
| | Bifidobacterium infantis |
| | 3-fucosyllactose |
| 219 | PB-STR-083 |
| | Bifidobacterium breve |
| | Bifidobacterium longum |
| | 3-fucosyllactose |
| 220 | PB-STR-093 |
| | Bifidobacterium longum |
| | Bifidobacterium breve |
| | Bifidobacterium infantis |
| | 3-fucosyllactose |
| 221 | PB-STR-207 |
| | Bifidobacterium longum |
| | Bifidobacterium breve |
| | Bifidobacterium infantis |
| | 3-fucosyllactose |
| 222 | PB-STR-119 |
| | Bifidobacterium longum |
| | Bifidobacterium breve |
| | Bifidobacterium infantis |
| | 3-fucosyllactose |
| 223 | PB-STR-083 |
| | Bifidobacterium longum |
| | Bifidobacterium breve |
| | Bifidobacterium infantis |
| | 3-fucosyllactose |
| 224 | PB-STR-093 |
| | PB-STR-207 |
| | PB-STR-119 |
| | PB-STR-083 |
| | 3-fucosyllactose |
| 225 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 3-fucosyllactose |
| 226 | PB-STR-207 |
| | PB-STR-215 |
| | PB-STR-220 |
| | 3-fucosyllactose |
| 227 | PB-STR-003 |
| | PB-STR-093 |
| | PB-STR-034 |
| | PB-STR-035 |
| | PB-STR-083 |
| | 3-fucosyllactose |
| 228 | PB-STR-119 |
| | PB-STR-103 |
| | 3-fucosyllactose |
| 229 | PB-STR-093 |
| | 2′-fucosyllactose |
| | Lacto-N-tetraose |
| 230 | PB-STR-207 |
| | 2′-fucosyllactose |
| | Lacto-N-tetraose |
| 231 | PB-STR-083 |
| | 2′-fucosyllactose |
| | Lacto-N-tetraose |
| 232 | PB-STR-119 |
| | 2′-fucosyllactose |
| | Lacto-N-tetraose |
| 233 | PB-STR-093 |
| | Bifidobacterium infantis |
| | 2′-fucosyllactose |
| | Lacto-N-tetraose |
| 234 | PB-STR-093 |
| | Bifidobacterium longum |
| | 2′-fucosyllactose |
| | Lacto-N-tetraose |
| 235 | PB-STR-093 |
| | Bifidobacterium breve |
| | 2′-fucosyllactose |
| | Lacto-N-tetraose |
| 236 | PB-STR-207 |
| | Bifidobacterium infantis |
| | 2′-fucosyllactose |
| | Lacto-N-tetraose |
| 237 | PB-STR-207 |
| | Bifidobacterium longum |
| | 2′-fucosyllactose |
| | Lacto-N-tetraose |
| 238 | PB-STR-207 |
| | Bifidobacterium breve |
| | 2′-fucosyllactose |
| | Lacto-N-tetraose |
| 239 | PB-STR-207 |
| | Bifidobacterium bifidum |
| | 2′-fucosyllactose |
| | Lacto-N-tetraose |
| 240 | PB-STR-083 |
| | Bifidobacterium infantis |
| | 2′-fucosyllactose |
| | Lacto-N-tetraose |
| 241 | PB-STR-083 |
| | Bifidobacterium longum |
| | 2′-fucosyllactose |
| | Lacto-N-tetraose |
| 242 | PB-STR-083 |
| | Bifidobacterium breve |
| | 2′-fucosyllactose |
| | Lacto-N-tetraose |
| 243 | PB-STR-119 |
| | Bifidobacterium infantis |
| | 2′-fucosyllactose |
| | Lacto-N-tetraose |
| 244 | PB-STR-119 |
| | Bifidobacterium longum |
| | 2′-fucosyllactose |
| | Lacto-N-tetraose |
| 245 | PB-STR-119 |
| | Bifidobacterium breve |
| | 2′-fucosyllactose |
| | Lacto-N-tetraose |
| 246 | PB-STR-093 |
| | Bifidobacterium longum |
| | Bifidobacterium infantis |
| | 2′-fucosyllactose |
| | Lacto-N-tetraose |
| 247 | PB-STR-093 |
| | Bifidobacterium breve |
| | Bifidobacterium infantis |
| | 2′-fucosyllactose |
| | Lacto-N-tetraose |
| 248 | PB-STR-093 |
| | Bifidobacterium breve |
| | Bifidobacterium longum |
| | 2′-fucosyllactose |
| | Lacto-N-tetraose |
| 249 | PB-STR-207 |
| | Bifidobacterium longum |
| | Bifidobacterium infantis |
| | 2′-fucosyllactose |
| | Lacto-N-tetraose |
| 250 | PB-STR-207 |
| | Bifidobacterium breve |
| | Bifidobacterium infantis |
| | 2′-fucosyllactose |
| | Lacto-N-tetraose |
| 251 | PB-STR-207 |
| | Bifidobacterium breve |
| | Bifidobacterium longum |
| | 2′-fucosyllactose |
| | Lacto-N-tetraose |
| 252 | PB-STR-119 |
| | Bifidobacterium longum |
| | Bifidobacterium infantis |
| | 2′-fucosyllactose |
| | Lacto-N-tetraose |
| 253 | PB-STR-119 |
| | Bifidobacterium breve |
| | Bifidobacterium infantis |
| | 2′-fucosyllactose |
| | Lacto-N-tetraose |
| 254 | PB-STR-119 |
| | Bifidobacterium breve |
| | Bifidobacterium longum |
| | 2′-fucosyllactose |
| | Lacto-N-tetraose |
| 255 | PB-STR-083 |
| | Bifidobacterium longum |
| | Bifidobacterium infantis |
| | 2′-fucosyllactose |
| | Lacto-N-tetraose |
| 256 | PB-STR-083 |
| | Bifidobacterium breve |
| | Bifidobacterium infantis |
| | 2′-fucosyllactose |
| | Lacto-N-tetraose |
| 257 | PB-STR-083 |
| | Bifidobacterium breve |
| | Bifidobacterium longum |
| | 2′-fucosyllactose |
| | Lacto-N-tetraose |
| 258 | PB-STR-093 |
| | Bifidobacterium longum |
| | Bifidobacterium breve |
| | Bifidobacterium infantis |
| | 2′-fucosyllactose |
| | Lacto-N-tetraose |
| 259 | PB-STR-207 |
| | Bifidobacterium longum |
| | Bifidobacterium breve |
| | Bifidobacterium infantis |
| | 2′-fucosyllactose |
| | Lacto-N-tetraose |
| 260 | PB-STR-119 |
| | Bifidobacterium longum |
| | Bifidobacterium breve |
| | Bifidobacterium infantis |
| | 2′-fucosyllactose |
| | Lacto-N-tetraose |
| 261 | PB-STR-083 |
| | Bifidobacterium longum |
| | Bifidobacterium breve |
| | Bifidobacterium infantis |
| | 2′-fucosyllactose |
| | Lacto-N-tetraose |
| 262 | PB-STR-093 |
| | PB-STR-207 |
| | PB-STR-119 |
| | PB-STR-083 |
| | 2′-fucosyllactose |
| | Lacto-N-tetraose |
| 263 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 2′-fucosyllactose |
| | Lacto-N-tetraose |
| 264 | PB-STR-207 |
| | PB-STR-215 |
| | PB-STR-220 |
| | 2′-fucosyllactose |
| | Lacto-N-tetraose |
| 265 | PB-STR-003 |
| | PB-STR-093 |
| | PB-STR-034 |
| | PB-STR-035 |
| | PB-STR-083 |
| | 2′-fucosyllactose |
| | Lacto-N-tetraose |
| 266 | PB-STR-119 |
| | PB-STR-103 |
| | 2′-fucosyllactose |
| | Lacto-N-tetraose |
| 267 | PB-STR-093 |
| | Lacto-N-tetraose |
| | Lacto-N-neotetraose |
| 268 | PB-STR-207 |
| | Lacto-N-tetraose |
| | Lacto-N-neotetraose |
| 269 | PB-STR-083 |
| | Lacto-N-tetraose |
| | Lacto-N-neotetraose |
| 270 | PB-STR-119 |
| | Lacto-N-tetraose |
| | Lacto-N-neotetraose |
| 271 | PB-STR-093 |
| | Bifidobacterium infantis |
| | Lacto-N-tetraose |
| | Lacto-N-neotetraose |
| 272 | PB-STR-093 |
| | Bifidobacterium longum |
| | Lacto-N-tetraose |
| | Lacto-N-neotetraose |
| 273 | PB-STR-093 |
| | Bifidobacterium breve |
| | Lacto-N-tetraose |
| | Lacto-N-neotetraose |
| 274 | PB-STR-207 |
| | Bifidobacterium infantis |
| | Lacto-N-tetraose |
| | Lacto-N-neotetraose |
| 275 | PB-STR-207 |
| | Bifidobacterium longum |
| | Lacto-N-tetraose |
| | Lacto-N-neotetraose |
| 276 | PB-STR-207 |
| | Bifidobacterium breve |
| | Lacto-N-tetraose |
| | Lacto-N-neotetraose |
| 277 | PB-STR-207 |
| | Bifidobacterium bifidum |
| | Lacto-N-tetraose |
| | Lacto-N-neotetraose |
| 278 | PB-STR-083 |
| | Bifidobacterium infantis |
| | Lacto-N-tetraose |
| | Lacto-N-neotetraose |
| 279 | PB-STR-083 |
| | Bifidobacterium longum |
| | Lacto-N-tetraose |
| | Lacto-N-neotetraose |
| 280 | PB-STR-083 |
| | Bifidobacterium breve |
| | Lacto-N-tetraose |
| | Lacto-N-neotetraose |
| 281 | PB-STR-119 |
| | Bifidobacterium infantis |
| | Lacto-N-tetraose |
| | Lacto-N-neotetraose |
| 282 | PB-STR-119 |
| | Bifidobacterium longum |
| | Lacto-N-tetraose |
| | Lacto-N-neotetraose |
| 283 | PB-STR-119 |
| | Bifidobacterium breve |
| | Lacto-N-tetraose |
| | Lacto-N-neotetraose |
| 284 | PB-STR-093 |
| | Bifidobacterium longum |
| | Bifidobacterium infantis |
| | Lacto-N-tetraose |
| | Lacto-N-neotetraose |
| 285 | PB-STR-093 |
| | Bifidobacterium breve |
| | Bifidobacterium infantis |
| | Lacto-N-tetraose |
| | Lacto-N-neotetraose |
| 286 | PB-STR-093 |
| | Bifidobacterium breve |
| | Bifidobacterium longum |
| | Lacto-N-tetraose |
| | Lacto-N-neotetraose |
| 287 | PB-STR-207 |
| | Bifidobacterium longum |
| | Bifidobacterium infantis |
| | Lacto-N-tetraose |
| | Lacto-N-neotetraose |
| 288 | PB-STR-207 |
| | Bifidobacterium breve |
| | Bifidobacterium infantis |
| | Lacto-N-tetraose |
| | Lacto-N-neotetraose |
| 289 | PB-STR-207 |
| | Bifidobacterium breve |
| | Bifidobacterium longum |
| | Lacto-N-tetraose |
| | Lacto-N-neotetraose |
| 290 | PB-STR-119 |
| | Bifidobacterium longum |
| | Bifidobacterium infantis |
| | Lacto-N-tetraose |
| | Lacto-N-neotetraose |
| 291 | PB-STR-119 |
| | Bifidobacterium breve |
| | Bifidobacterium infantis |
| | Lacto-N-tetraose |
| | Lacto-N-neotetraose |
| 292 | PB-STR-119 |
| | Bifidobacterium breve |
| | Bifidobacterium longum |
| | Lacto-N-tetraose |
| | Lacto-N-neotetraose |
| 293 | PB-STR-083 |
| | Bifidobacterium longum |
| | Bifidobacterium infantis |
| | Lacto-N-tetraose |
| | Lacto-N-neotetraose |
| 294 | PB-STR-083 |
| | Bifidobacterium breve |
| | Bifidobacterium infantis |
| | Lacto-N-tetraose |
| | Lacto-N-neotetraose |
| 295 | PB-STR-083 |
| | Bifidobacterium breve |
| | Bifidobacterium longum |
| | Lacto-N-tetraose |
| | Lacto-N-neotetraose |
| 296 | PB-STR-093 |
| | Bifidobacterium longum |
| | Bifidobacterium breve |
| | Bifidobacterium infantis |
| | Lacto-N-tetraose |
| | Lacto-N-neotetraose |
| 297 | PB-STR-207 |
| | Bifidobacterium longum |
| | Bifidobacterium breve |
| | Bifidobacterium infantis |
| | Lacto-N-tetraose |
| | Lacto-N-neotetraose |
| 298 | PB-STR-119 |
| | Bifidobacterium longum |
| | Bifidobacterium breve |
| | Bifidobacterium infantis |
| | Lacto-N-tetraose |
| | Lacto-N-neotetraose |
| 299 | PB-STR-083 |
| | Bifidobacterium longum |
| | Bifidobacterium breve |
| | Bifidobacterium infantis |
| | Lacto-N-tetraose |
| | Lacto-N-neotetraose |
| 300 | PB-STR-093 |
| | PB-STR-207 |
| | PB-STR-119 |
| | PB-STR-083 |
| | Lacto-N-tetraose |
| | Lacto-N-neotetraose |
| 301 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | Lacto-N-tetraose |
| | Lacto-N-neotetraose |
| 302 | PB-STR-207 |
| | PB-STR-215 |
| | PB-STR-220 |
| | Lacto-N-tetraose |
| | Lacto-N-neotetraose |
| 303 | PB-STR-003 |
| | PB-STR-093 |
| | PB-STR-034 |
| | PB-STR-035 |
| | PB-STR-083 |
| | Lacto-N-tetraose |
| | Lacto-N-neotetraose |
| 304 | PB-STR-119 |
| | PB-STR-103 |
| | Lacto-N-tetraose |
| | Lacto-N-neotetraose |
| 305 | PB-STR-093 |
| | 2′-fucosyllactose |
| | 3-fucosyllactose |
| 306 | PB-STR-207 |
| | 2′-fucosyllactose |
| | 3-fucosyllactose |
| 307 | PB-STR-083 |
| | 2′-fucosyllactose |
| | 3-fucosyllactose |
| 308 | PB-STR-119 |
| | 2′-fucosyllactose |
| | 3-fucosyllactose |
| 309 | PB-STR-093 |
| | Bifidobacterium infantis |
| | 2′-fucosyllactose |
| | 3-fucosyllactose |
| 310 | PB-STR-093 |
| | Bifidobacterium longum |
| | 2′-fucosyllactose |
| | 3-fucosyllactose |
| 311 | PB-STR-093 |
| | Bifidobacterium breve |
| | 2′-fucosyllactose |
| | 3-fucosyllactose |
| 312 | PB-STR-207 |
| | Bifidobacterium infantis |
| | 2′-fucosyllactose |
| | 3-fucosyllactose |
| 313 | PB-STR-207 |
| | Bifidobacterium longum |
| | 2′-fucosyllactose |
| | 3-fucosyllactose |
| 314 | PB-STR-207 |
| | Bifidobacterium breve |
| | 2′-fucosyllactose |
| | 3-fucosyllactose |
| 315 | PB-STR-207 |
| | Bifidobacterium bifidum |
| | 2′-fucosyllactose |
| | 3-fucosyllactose |
| 316 | PB-STR-083 |
| | Bifidobacterium infantis |
| | 2′-fucosyllactose |
| | 3-fucosyllactose |
| 317 | PB-STR-083 |
| | Bifidobacterium longum |
| | 2′-fucosyllactose |
| | 3-fucosyllactose |
| 318 | PB-STR-083 |
| | Bifidobacterium breve |
| | 2′-fucosyllactose |
| | 3-fucosyllactose |
| 319 | PB-STR-119 |
| | Bifidobacterium infantis |
| | 2′-fucosyllactose |
| | 3-fucosyllactose |
| 320 | PB-STR-119 |
| | Bifidobacterium longum |
| | 2′-fucosyllactose |
| | 3-fucosyllactose |
| 321 | PB-STR-119 |
| | Bifidobacterium breve |
| | 2′-fucosyllactose |
| | 3-fucosyllactose |
| 322 | PB-STR-093 |
| | Bifidobacterium longum |
| | Bifidobacterium infantis |
| | 2′-fucosyllactose |
| | 3-fucosyllactose |
| 323 | PB-STR-093 |
| | Bifidobacterium breve |
| | Bifidobacterium infantis |
| | 2′-fucosyllactose |
| | 3-fucosyllactose |
| 324 | PB-STR-093 |
| | Bifidobacterium breve |
| | Bifidobacterium longum |
| | 2′-fucosyllactose |
| | 3-fucosyllactose |
| 325 | PB-STR-207 |
| | Bifidobacterium longum |
| | Bifidobacterium infantis |
| | 2′-fucosyllactose |
| | 3-fucosyllactose |
| 326 | PB-STR-207 |
| | Bifidobacterium breve |
| | Bifidobacterium infantis |
| | 2′-fucosyllactose |
| | 3-fucosyllactose |
| 327 | PB-STR-207 |
| | Bifidobacterium breve |
| | Bifidobacterium longum |
| | 2′-fucosyllactose |
| | 3-fucosyllactose |
| 328 | PB-STR-119 |
| | Bifidobacterium longum |
| | Bifidobacterium infantis |
| | 2′-fucosyllactose |
| | 3-fucosyllactose |
| 329 | PB-STR-119 |
| | Bifidobacterium breve |
| | Bifidobacterium infantis |
| | 2′-fucosyllactose |
| | 3-fucosyllactose |
| 330 | PB-STR-119 |
| | Bifidobacterium breve |
| | Bifidobacterium longum |
| | 2′-fucosyllactose |
| | 3-fucosyllactose |
| 331 | PB-STR-083 |
| | Bifidobacterium longum |
| | Bifidobacterium infantis |
| | 2′-fucosyllactose |
| | 3-fucosyllactose |
| 332 | PB-STR-083 |
| | Bifidobacterium breve |
| | Bifidobacterium infantis |
| | 2′-fucosyllactose |
| | 3-fucosyllactose |
| 333 | PB-STR-083 |
| | Bifidobacterium breve |
| | Bifidobacterium longum |
| | 2′-fucosyllactose |
| | 3-fucosyllactose |
| 331 | PB-STR-093 |
| | Bifidobacterium longum |
| | Bifidobacterium breve |
| | Bifidobacterium infantis |
| | 2′-fucosyllactose |
| | 3-fucosyllactose |
| 332 | PB-STR-207 |
| | Bifidobacterium longum |
| | Bifidobacterium breve |
| | Bifidobacterium infantis |
| | 2′-fucosyllactose |
| | 3-fucosyllactose |
| 333 | PB-STR-119 |
| | Bifidobacterium longum |
| | Bifidobacterium breve |
| | Bifidobacterium infantis |
| | 2′-fucosyllactose |
| | 3-fucosyllactose |
| 334 | PB-STR-083 |
| | Bifidobacterium longum |
| | Bifidobacterium breve |
| | Bifidobacterium infantis |
| | 2′-fucosyllactose |
| | 3-fucosyllactose |
| 335 | PB-STR-093 |
| | PB-STR-207 |
| | PB-STR-119 |
| | PB-STR-083 |
| | 2′-fucosyllactose |
| | 3-fucosyllactose |
| 336 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 2′-fucosyllactose |
| | 3-fucosyllactose |
| 337 | PB-STR-207 |
| | PB-STR-215 |
| | PB-STR-220 |
| | 2′-fucosyllactose |
| | 3-fucosyllactose |
| 338 | PB-STR-003 |
| | PB-STR-093 |
| | PB-STR-034 |
| | PB-STR-035 |
| | PB-STR-083 |
| | 2′-fucosyllactose |
| | 3-fucosyllactose |
| 339 | PB-STR-119 |
| | PB-STR-103 |
| | 2′-fucosyllactose |
| | 3-fucosyllactose |
| 340 | PB-STR-093 |
| | 3′-sialyllactose |
| | 6′-sialyllactose |
| 341 | PB-STR-207 |
| | 3′-sialyllactose |
| | 6′-sialyllactose |
| 342 | PB-STR-083 |
| | 3′-sialyllactose |
| | 6′-sialyllactose |
| 343 | PB-STR-119 |
| | 3′-sialyllactose |
| | 6′-sialyllactose |
| 344 | PB-STR-093 |
| | Bifidobacterium infantis |
| | 3′-sialyllactose |
| | 6′-sialyllactose |
| 345 | PB-STR-093 |
| | Bifidobacterium longum |
| | 3′-sialyllactose |
| | 6′-sialyllactose |
| 346 | PB-STR-093 |
| | Bifidobacterium breve |
| | 3′-sialyllactose |
| | 6′-sialyllactose |
| 347 | PB-STR-207 |
| | Bifidobacterium infantis |
| | 3′-sialyllactose |
| | 6′-sialyllactose |
| 348 | PB-STR-207 |
| | Bifidobacterium longum |
| | 3′-sialyllactose |
| | 6′-sialyllactose |
| 349 | PB-STR-207 |
| | Bifidobacterium breve |
| | 3′-sialyllactose |
| | 6′-sialyllactose |
| 350 | PB-STR-207 |
| | Bifidobacterium bifidum |
| | 3′-sialyllactose |
| | 6′-sialyllactose |
| 351 | PB-STR-083 |
| | Bifidobacterium infantis |
| | 3′-sialyllactose |
| | 6′-sialyllactose |
| 352 | PB-STR-083 |
| | Bifidobacterium longum |
| | 3′-sialyllactose |
| | 6′-sialyllactose |
| 353 | PB-STR-083 |
| | Bifidobacterium breve |
| | 3′-sialyllactose |
| | 6′-sialyllactose |
| 354 | PB-STR-119 |
| | Bifidobacterium infantis |
| | 3′-sialyllactose |
| | 6′-sialyllactose |
| 355 | PB-STR-119 |
| | Bifidobacterium longum |
| | 3′-sialyllactose |
| | 6′-sialyllactose |
| 356 | PB-STR-119 |
| | Bifidobacterium breve |
| | 3′-sialyllactose |
| | 6′-sialyllactose |
| 357 | PB-STR-093 |
| | Bifidobacterium longum |
| | Bifidobacterium infantis |
| | 3′-sialyllactose |
| | 6′-sialyllactose |
| 358 | PB-STR-093 |
| | Bifidobacterium breve |
| | Bifidobacterium infantis |
| | 3′-sialyllactose |
| | 6′-sialyllactose |
| 359 | PB-STR-093 |
| | Bifidobacterium breve |
| | Bifidobacterium longum |
| | 3′-sialyllactose |
| | 6′-sialyllactose |
| 360 | PB-STR-207 |
| | Bifidobacterium longum |
| | Bifidobacterium infantis |
| | 3′-sialyllactose |
| | 6′-sialyllactose |
| 361 | PB-STR-207 |
| | Bifidobacterium breve |
| | Bifidobacterium infantis |
| | 3′-sialyllactose |
| | 6′-sialyllactose |
| 362 | PB-STR-207 |
| | Bifidobacterium breve |
| | Bifidobacterium longum |
| | 3′-sialyllactose |
| | 6′-sialyllactose |
| 363 | PB-STR-119 |
| | Bifidobacterium longum |
| | Bifidobacterium infantis |
| | 3′-sialyllactose |
| | 6′-sialyllactose |
| 364 | PB-STR-119 |
| | Bifidobacterium breve |
| | Bifidobacterium infantis |
| | 3′-sialyllactose |
| | 6′-sialyllactose |
| 365 | PB-STR-119 |
| | Bifidobacterium breve |
| | Bifidobacterium longum |
| | 3′-sialyllactose |
| | 6′-sialyllactose |
| 366 | PB-STR-083 |
| | Bifidobacterium longum |
| | Bifidobacterium infantis |
| | 3′-sialyllactose |
| | 6′-sialyllactose |
| 367 | PB-STR-083 |
| | Bifidobacterium breve |
| | Bifidobacterium infantis |
| | 3′-sialyllactose |
| | 6′-sialyllactose |
| 368 | PB-STR-083 |
| | Bifidobacterium breve |
| | Bifidobacterium longum |
| | 3′-sialyllactose |
| | 6′-sialyllactose |
| 369 | PB-STR-093 |
| | Bifidobacterium longum |
| | Bifidobacterium breve |
| | Bifidobacterium infantis |
| | 3′-sialyllactose |
| | 6′-sialyllactose |
| 370 | PB-STR-207 |
| | Bifidobacterium longum |
| | Bifidobacterium breve |
| | Bifidobacterium infantis |
| | 3′-sialyllactose |
| | 6′-sialyllactose |
| 371 | PB-STR-119 |
| | Bifidobacterium longum |
| | Bifidobacterium breve |
| | Bifidobacterium infantis |
| | 3′-sialyllactose |
| | 6′-sialyllactose |
| 372 | PB-STR-083 |
| | Bifidobacterium longum |
| | Bifidobacterium breve |
| | Bifidobacterium infantis |
| | 3′-sialyllactose |
| | 6′-sialyllactose |
| 373 | PB-STR-093 |
| | PB-STR-207 |
| | PB-STR-119 |
| | PB-STR-083 |
| | 3′-sialyllactose |
| | 6′-sialyllactose |
| 374 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 3′-sialyllactose |
| | 6′-sialyllactose |
| 375 | PB-STR-207 |
| | PB-STR-215 |
| | PB-STR-220 |
| | 3′-sialyllactose |
| | 6′-sialyllactose |
| 376 | PB-STR-003 |
| | PB-STR-093 |
| | PB-STR-034 |
| | PB-STR-035 |
| | PB-STR-083 |
| | 3′-sialyllactose |
| | 6′-sialyllactose |
| 377 | PB-STR-119 |
| | PB-STR-103 |
| | 3′-sialyllactose |
| | 6′-sialyllactose |
| 378 | PB-STR-093 |
| | 2′-fucosyllactose |
| | 3′-sialyllactose |
| 379 | PB-STR-207 |
| | 2′-fucosyllactose |
| | 3′-sialyllactose |
| 380 | PB-STR-083 |
| | 2′-fucosyllactose |
| | 3′-sialyllactose |
| 381 | PB-STR-119 |
| | 2′-fucosyllactose |
| | 3′-sialyllactose |
| 382 | PB-STR-093 |
| | Bifidobacterium infantis |
| | 2′-fucosyllactose |
| | 3′-sialyllactose |
| 383 | PB-STR-093 |
| | Bifidobacterium longum |
| | 2′-fucosyllactose |
| | 3′-sialyllactose |
| 384 | PB-STR-093 |
| | Bifidobacterium breve |
| | 2′-fucosyllactose |
| | 3′-sialyllactose |
| 385 | PB-STR-207 |
| | Bifidobacterium infantis |
| | 2′-fucosyllactose |
| | 3′-sialyllactose |
| 386 | PB-STR-207 |
| | Bifidobacterium longum |
| | 2′-fucosyllactose |
| | 3′-sialyllactose |
| 387 | PB-STR-207 |
| | Bifidobacterium breve |
| | 2′-fucosyllactose |
| | 3′-sialyllactose |
| 388 | PB-STR-207 |
| | Bifidobacterium bifidum |
| | 2′-fucosyllactose |
| | 3′-sialyllactose |
| 389 | PB-STR-083 |
| | Bifidobacterium infantis |
| | 2′-fucosyllactose |
| | 3′-sialyllactose |
| 390 | PB-STR-083 |
| | Bifidobacterium longum |
| | 2′-fucosyllactose |
| | 3′-sialyllactose |
| 391 | PB-STR-083 |
| | Bifidobacterium breve |
| | 2′-fucosyllactose |
| | 3′-sialyllactose |
| 392 | PB-STR-119 |
| | Bifidobacterium infantis |
| | 2′-fucosyllactose |
| | 3′-sialyllactose |
| 393 | PB-STR-119 |
| | Bifidobacterium longum |
| | 2′-fucosyllactose |
| | 3′-sialyllactose |
| 394 | PB-STR-119 |
| | Bifidobacterium breve |
| | 2′-fucosyllactose |
| | 3′-sialyllactose |
| 395 | PB-STR-093 |
| | Bifidobacterium longum |
| | Bifidobacterium infantis |
| | 2′-fucosyllactose |
| | 3′-sialyllactose |
| 396 | PB-STR-093 |
| | Bifidobacterium breve |
| | Bifidobacterium infantis |
| | 2′-fucosyllactose |
| | 3′-sialyllactose |
| 397 | PB-STR-093 |
| | Bifidobacterium breve |
| | Bifidobacterium longum |
| | 2′-fucosyllactose |
| | 3′-sialyllactose |
| 398 | PB-STR-207 |
| | Bifidobacterium longum |
| | Bifidobacterium infantis |
| | 2′-fucosyllactose |
| | 3′-sialyllactose |
| 399 | PB-STR-207 |
| | Bifidobacterium breve |
| | Bifidobacterium infantis |
| | 2′-fucosyllactose |
| | 3′-sialyllactose |
| 400 | PB-STR-207 |
| | Bifidobacterium breve |
| | Bifidobacterium longum |
| | 2′-fucosyllactose |
| | 3′-sialyllactose |
| 401 | PB-STR-119 |
| | Bifidobacterium longum |
| | Bifidobacterium infantis |
| | 2′-fucosyllactose |
| | 3′-sialyllactose |
| 402 | PB-STR-119 |
| | Bifidobacterium breve |
| | Bifidobacterium infantis |
| | 2′-fucosyllactose |
| | 3′-sialyllactose |
| 403 | PB-STR-119 |
| | Bifidobacterium breve |
| | Bifidobacterium longum |
| | 2′-fucosyllactose |
| | 3′-sialyllactose |
| 404 | PB-STR-083 |
| | Bifidobacterium longum |
| | Bifidobacterium infantis |
| | 2′-fucosyllactose |
| | 3′-sialyllactose |
| 405 | PB-STR-083 |
| | Bifidobacterium breve |
| | Bifidobacterium infantis |
| | 2′-fucosyllactose |
| | 3′-sialyllactose |
| 406 | PB-STR-083 |
| | Bifidobacterium breve |
| | Bifidobacterium longum |
| | 2′-fucosyllactose |
| | 3′-sialyllactose |
| 407 | PB-STR-093 |
| | Bifidobacterium longum |
| | Bifidobacterium breve |
| | Bifidobacterium infantis |
| | 2′-fucosyllactose |
| | 3′-sialyllactose |
| 408 | PB-STR-207 |
| | Bifidobacterium longum |
| | Bifidobacterium breve |
| | Bifidobacterium infantis |
| | 2′-fucosyllactose |
| | 3′-sialyllactose |
| 409 | PB-STR-119 |
| | Bifidobacterium longum |
| | Bifidobacterium breve |
| | Bifidobacterium infantis |
| | 2′-fucosyllactose |
| | 3′-sialyllactose |
| 372 | PB-STR-083 |
| | Bifidobacterium longum |
| | Bifidobacterium breve |
| | Bifidobacterium infantis |
| | 2′-fucosyllactose |
| | 3′-sialyllactose |
| 373 | PB-STR-093 |
| | PB-STR-207 |
| | PB-STR-119 |
| | PB-STR-083 |
| | 2′-fucosyllactose |
| | 3′-sialyllactose |
| 374 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 2′-fucosyllactose |
| | 3′-sialyllactose |
| 375 | PB-STR-207 |
| | PB-STR-215 |
| | PB-STR-220 |
| | 2′-fucosyllactose |
| | 3′-sialyllactose |
| 376 | PB-STR-003 |
| | PB-STR-093 |
| | PB-STR-034 |
| | PB-STR-035 |
| | PB-STR-083 |
| | 2′-fucosyllactose |
| | 3′-sialyllactose |
| 377 | PB-STR-119 |
| | PB-STR-103 |
| | 2′-fucosyllactose |
| | 3′-sialyllactose |
| 378 | PB-STR-093 |
| | Lacto-N-tetraose |
| | 3′-sialyllactose |
| 379 | PB-STR-207 |
| | Lacto-N-tetraose |
| | 3′-sialyllactose |
| 380 | PB-STR-083 |
| | Lacto-N-tetraose |
| | 3′-sialyllactose |
| 381 | PB-STR-119 |
| | Lacto-N-tetraose |
| | 3′-sialyllactose |
| 382 | PB-STR-093 |
| | Bifidobacterium infantis |
| | Lacto-N-tetraose |
| | 3′-sialyllactose |
| 383 | PB-STR-093 |
| | Bifidobacterium longum |
| | Lacto-N-tetraose |
| | 3′-sialyllactose |
| 384 | PB-STR-093 |
| | Bifidobacterium breve |
| | Lacto-N-tetraose |
| | 3′-sialyllactose |
| 385 | PB-STR-207 |
| | Bifidobacterium infantis |
| | Lacto-N-tetraose |
| | 3′-sialyllactose |
| 386 | PB-STR-207 |
| | Bifidobacterium longum |
| | Lacto-N-tetraose |
| | 3′-sialyllactose |
| 387 | PB-STR-207 |
| | Bifidobacterium breve |
| | Lacto-N-tetraose |
| | 3′-sialyllactose |
| 388 | PB-STR-207 |
| | Bifidobacterium bifidum |
| | Lacto-N-tetraose |
| | 3′-sialyllactose |
| 389 | PB-STR-083 |
| | Bifidobacterium infantis |
| | Lacto-N-tetraose |
| | 3′-sialyllactose |
| 390 | PB-STR-083 |
| | Bifidobacterium longum |
| | Lacto-N-tetraose |
| | 3′-sialyllactose |
| 391 | PB-STR-083 |
| | Bifidobacterium breve |
| | Lacto-N-tetraose |
| | 3′-sialyllactose |
| 392 | PB-STR-119 |
| | Bifidobacterium infantis |
| | Lacto-N-tetraose |
| | 3′-sialyllactose |
| 393 | PB-STR-119 |
| | Bifidobacterium longum |
| | Lacto-N-tetraose |
| | 3′-sialyllactose |
| 394 | PB-STR-119 |
| | Bifidobacterium breve |
| | Lacto-N-tetraose |
| | 3′-sialyllactose |
| 395 | PB-STR-093 |
| | Bifidobacterium longum |
| | Bifidobacterium infantis |
| | Lacto-N-tetraose |
| | 3′-sialyllactose |
| 396 | PB-STR-093 |
| | Bifidobacterium breve |
| | Bifidobacterium infantis |
| | Lacto-N-tetraose |
| | 3′-sialyllactose |
| 397 | PB-STR-093 |
| | Bifidobacterium breve |
| | Bifidobacterium longum |
| | Lacto-N-tetraose |
| | 3′-sialyllactose |
| 398 | PB-STR-207 |
| | Bifidobacterium longum |
| | Bifidobacterium infantis |
| | Lacto-N-tetraose |
| | 3′-sialyllactose |
| 399 | PB-STR-207 |
| | Bifidobacterium breve |
| | Bifidobacterium infantis |
| | Lacto-N-tetraose |
| | 3′-sialyllactose |
| 400 | PB-STR-207 |
| | Bifidobacterium breve |
| | Bifidobacterium longum |
| | Lacto-N-tetraose |
| | 3′-sialyllactose |
| 401 | PB-STR-119 |
| | Bifidobacterium longum |
| | Bifidobacterium infantis |
| | Lacto-N-tetraose |
| | 3′-sialyllactose |
| 402 | PB-STR-119 |
| | Bifidobacterium breve |
| | Bifidobacterium infantis |
| | Lacto-N-tetraose |
| | 3′-sialyllactose |
| 403 | PB-STR-119 |
| | Bifidobacterium breve |
| | Bifidobacterium longum |
| | Lacto-N-tetraose |
| | 3′-sialyllactose |
| 404 | PB-STR-083 |
| | Bifidobacterium longum |
| | Bifidobacterium infantis |
| | Lacto-N-tetraose |
| | 3′-sialyllactose |
| 405 | PB-STR-083 |
| | Bifidobacterium breve |
| | Bifidobacterium infantis |
| | Lacto-N-tetraose |
| | 3′-sialyllactose |
| 406 | PB-STR-083 |
| | Bifidobacterium breve |
| | Bifidobacterium longum |
| | Lacto-N-tetraose |
| | 3′-sialyllactose |
| 407 | PB-STR-093 |
| | Bifidobacterium longum |
| | Bifidobacterium breve |
| | Bifidobacterium infantis |
| | Lacto-N-tetraose |
| | 3′-sialyllactose |
| 408 | PB-STR-207 |
| | Bifidobacterium longum |
| | Bifidobacterium breve |
| | Bifidobacterium infantis |
| | Lacto-N-tetraose |
| | 3′-sialyllactose |
| 409 | PB-STR-119 |
| | Bifidobacterium longum |
| | Bifidobacterium breve |
| | Bifidobacterium infantis |
| | Lacto-N-tetraose |
| | 3′-sialyllactose |
| 410 | PB-STR-083 |
| | Bifidobacterium longum |
| | Bifidobacterium breve |
| | Bifidobacterium infantis |
| | Lacto-N-tetraose |
| | 3′-sialyllactose |
| 411 | PB-STR-093 |
| | PB-STR-207 |
| | PB-STR-119 |
| | PB-STR-083 |
| | Lacto-N-tetraose |
| | 3′-sialyllactose |
| 412 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | Lacto-N-tetraose |
| | 3′-sialyllactose |
| 413 | PB-STR-207 |
| | PB-STR-215 |
| | PB-STR-220 |
| | Lacto-N-tetraose |
| | 3′-sialyllactose |
| 414 | PB-STR-003 |
| | PB-STR-093 |
| | PB-STR-034 |
| | PB-STR-035 |
| | PB-STR-083 |
| | Lacto-N-tetraose |
| | 3′-sialyllactose |
| 415 | PB-STR-119 |
| | PB-STR-103 |
| | Lacto-N-tetraose |
| | 3′-sialyllactose |
| 416 | PB-STR-093 |
| | 2′-fucosyllactose |
| | Lacto-N-tetraose |
| | 3′-sialyllactose |
| 417 | PB-STR-207 |
| | 2′-fucosyllactose |
| | Lacto-N-tetraose |
| | 3′-sialyllactose |
| 418 | PB-STR-083 |
| | 2′-fucosyllactose |
| | Lacto-N-tetraose |
| | 3′-sialyllactose |
| 419 | PB-STR-119 |
| | 2′-fucosyllactose |
| | Lacto-N-tetraose |
| | 3′-sialyllactose |
| 420 | PB-STR-093 |
| | Bifidobacterium infantis |
| | 2′-fucosyllactose |
| | Lacto-N-tetraose |
| | 3′-sialyllactose |
| 421 | PB-STR-093 |
| | Bifidobacterium longum |
| | 2′-fucosyllactose |
| | Lacto-N-tetraose |
| | 3′-sialyllactose |
| 422 | PB-STR-093 |
| | Bifidobacterium breve |
| | 2′-fucosyllactose |
| | Lacto-N-tetraose |
| | 3′-sialyllactose |
| 423 | PB-STR-207 |
| | Bifidobacterium infantis |
| | 2′-fucosyllactose |
| | Lacto-N-tetraose |
| | 3′-sialyllactose |
| 424 | PB-STR-207 |
| | Bifidobacterium longum |
| | 2′-fucosyllactose |
| | Lacto-N-tetraose |
| | 3′-sialyllactose |
| 425 | PB-STR-207 |
| | Bifidobacterium breve |
| | 2′-fucosyllactose |
| | Lacto-N-tetraose |
| | 3′-sialyllactose |
| 426 | PB-STR-207 |
| | Bifidobacterium bifidum |
| | 2′-fucosyllactose |
| | Lacto-N-tetraose |
| | 3′-sialyllactose |
| 427 | PB-STR-083 |
| | Bifidobacterium infantis |
| | 2′-fucosyllactose |
| | Lacto-N-tetraose |
| | 3′-sialyllactose |
| 428 | PB-STR-083 |
| | Bifidobacterium longum |
| | 2′-fucosyllactose |
| | Lacto-N-tetraose |
| | 3′-sialyllactose |
| 429 | PB-STR-083 |
| | Bifidobacterium breve |
| | 2′-fucosyllactose |
| | Lacto-N-tetraose |
| | 3′-sialyllactose |
| 430 | PB-STR-119 |
| | Bifidobacterium infantis |
| | 2′-fucosyllactose |
| | Lacto-N-tetraose |
| | 3′-sialyllactose |
| 431 | PB-STR-119 |
| | Bifidobacterium longum |
| | 2′-fucosyllactose |
| | Lacto-N-tetraose |
| | 3′-sialyllactose |
| 432 | PB-STR-119 |
| | Bifidobacterium breve |
| | 2′-fucosyllactose |
| | Lacto-N-tetraose |
| | 3′-sialyllactose |
| 433 | PB-STR-093 |
| | Bifidobacterium longum |
| | Bifidobacterium infantis |
| | 2′-fucosyllactose |
| | Lacto-N-tetraose |
| | 3′-sialyllactose |
| 434 | PB-STR-093 |
| | Bifidobacterium breve |
| | Bifidobacterium infantis |
| | 2′-fucosyllactose |
| | Lacto-N-tetraose |
| | 3′-sialyllactose |
| 435 | PB-STR-093 |
| | Bifidobacterium breve |
| | Bifidobacterium longum |
| | 2′-fucosyllactose |
| | Lacto-N-tetraose |
| | 3′-sialyllactose |
| 436 | PB-STR-207 |
| | Bifidobacterium longum |
| | Bifidobacterium infantis |
| | 2′-fucosyllactose |
| | Lacto-N-tetraose |
| | 3′-sialyllactose |
| 437 | PB-STR-207 |
| | Bifidobacterium breve |
| | Bifidobacterium infantis |
| | 2′-fucosyllactose |
| | Lacto-N-tetraose |
| | 3′-sialyllactose |
| 438 | PB-STR-207 |
| | Bifidobacterium breve |
| | Bifidobacterium longum |
| | 2′-fucosyllactose |
| | Lacto-N-tetraose |
| | 3′-sialyllactose |
| 439 | PB-STR-119 |
| | Bifidobacterium longum |
| | Bifidobacterium infantis |
| | 2′-fucosyllactose |
| | Lacto-N-tetraose |
| | 3′-sialyllactose |
| 440 | PB-STR-119 |
| | Bifidobacterium breve |
| | Bifidobacterium infantis |
| | 2′-fucosyllactose |
| | Lacto-N-tetraose |
| | 3′-sialyllactose |
| 441 | PB-STR-119 |
| | Bifidobacterium breve |
| | Bifidobacterium longum |
| | 2′-fucosyllactose |
| | Lacto-N-tetraose |
| | 3′-sialyllactose |
| 442 | PB-STR-083 |
| | Bifidobacterium longum |
| | Bifidobacterium infantis |
| | 2′-fucosyllactose |
| | Lacto-N-tetraose |
| | 3′-sialyllactose |
| 443 | PB-STR-083 |
| | Bifidobacterium breve |
| | Bifidobacterium infantis |
| | 2′-fucosyllactose |
| | Lacto-N-tetraose |
| | 3′-sialyllactose |
| 444 | PB-STR-083 |
| | Bifidobacterium breve |
| | Bifidobacterium longum |
| | 2′-fucosyllactose |
| | Lacto-N-tetraose |
| | 3′-sialyllactose |
| 445 | PB-STR-093 |
| | Bifidobacterium longum |
| | Bifidobacterium breve |
| | Bifidobacterium infantis |
| | 2′-fucosyllactose |
| | Lacto-N-tetraose |
| | 3′-sialyllactose |
| 446 | PB-STR-207 |
| | Bifidobacterium longum |
| | Bifidobacterium breve |
| | Bifidobacterium infantis |
| | 2′-fucosyllactose |
| | Lacto-N-tetraose |
| | 3′-sialyllactose |
| 447 | PB-STR-119 |
| | Bifidobacterium longum |
| | Bifidobacterium breve |
| | Bifidobacterium infantis |
| | 2′-fucosyllactose |
| | Lacto-N-tetraose |
| | 3′-sialyllactose |
| 448 | PB-STR-083 |
| | Bifidobacterium longum |
| | Bifidobacterium breve |
| | Bifidobacterium infantis |
| | 2′-fucosyllactose |
| | Lacto-N-tetraose |
| | 3′-sialyllactose |
| 449 | PB-STR-093 |
| | PB-STR-207 |
| | PB-STR-119 |
| | PB-STR-083 |
| | 2′-fucosyllactose |
| | Lacto-N-tetraose |
| | 3′-sialyllactose |
| 450 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | 2′-fucosyllactose |
| | Lacto-N-tetraose |
| | 3′-sialyllactose |
| 451 | PB-STR-207 |
| | PB-STR-215 |
| | PB-STR-220 |
| | 2′-fucosyllactose |
| | Lacto-N-tetraose |
| | 3′-sialyllactose |
| 452 | PB-STR-003 |
| | PB-STR-093 |
| | PB-STR-034 |
| | PB-STR-035 |
| | PB-STR-083 |
| | 2′-fucosyllactose |
| | Lacto-N-tetraose |
| | 3′-sialyllactose |
| 453 | PB-STR-119 |
| | PB-STR-103 |
| | 2′-fucosyllactose |
| | Lacto-N-tetraose |
| | 3′-sialyllactose |
| 454 | PB-STR-093 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 455 | PB-STR-207 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 456 | PB-STR-083 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 457 | PB-STR-119 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 458 | PB-STR-093 |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 459 | PB-STR-093 |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 460 | PB-STR-093 |
| | Bifidobacterium breve |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 461 | PB-STR-207 |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 462 | PB-STR-207 |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 463 | PB-STR-207 |
| | Bifidobacterium breve |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 464 | PB-STR-207 |
| | Bifidobacterium bifidum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 465 | PB-STR-083 |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 466 | PB-STR-083 |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 467 | PB-STR-083 |
| | Bifidobacterium breve |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 468 | PB-STR-119 |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 469 | PB-STR-119 |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 470 | PB-STR-119 |
| | Bifidobacterium breve |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 471 | PB-STR-093 |
| | Bifidobacterium longum |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 472 | PB-STR-093 |
| | Bifidobacterium breve |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 473 | PB-STR-093 |
| | Bifidobacterium breve |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 474 | PB-STR-207 |
| | Bifidobacterium longum |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 475 | PB-STR-207 |
| | Bifidobacterium breve |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 476 | PB-STR-207 |
| | Bifidobacterium breve |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 477 | PB-STR-119 |
| | Bifidobacterium longum |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 478 | PB-STR-119 |
| | Bifidobacterium breve |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 479 | PB-STR-119 |
| | Bifidobacterium breve |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 480 | PB-STR-083 |
| | Bifidobacterium longum |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 481 | PB-STR-083 |
| | Bifidobacterium breve |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 482 | PB-STR-083 |
| | Bifidobacterium breve |
| | Bifidobacterium longum |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 483 | PB-STR-093 |
| | Bifidobacterium longum |
| | Bifidobacterium breve |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 484 | PB-STR-207 |
| | Bifidobacterium longum |
| | Bifidobacterium breve |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 485 | PB-STR-119 |
| | Bifidobacterium longum |
| | Bifidobacterium breve |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 486 | PB-STR-083 |
| | Bifidobacterium longum |
| | Bifidobacterium breve |
| | Bifidobacterium infantis |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 487 | PB-STR-093 |
| | PB-STR-207 |
| | PB-STR-119 |
| | PB-STR-083 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 488 | PB-STR-207 |
| | PB-STR-215 |
| | PB-STR-220 |
| | PB-STR-003 |
| | PB-STR-093 |
| | PB-STR-034 |
| | PB-STR-035 |
| | PB-STR-083 |
| | PB-STR-119 |
| | PB-STR-103 |
| | PB-STR-321 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 489 | PB-STR-207 |
| | PB-STR-215 |
| | PB-STR-220 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 490 | PB-STR-003 |
| | PB-STR-093 |
| | PB-STR-034 |
| | PB-STR-035 |
| | PB-STR-083 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |
| 491 | PB-STR-119 |
| | PB-STR-103 |
| | Lacto-N-tetraose |
| | Lacto-N-neotetraose |
| | 2′-fucosyllactose |
| | 3′-sialyllactose |
| | 6′-sialyllactose |
| | 3-fucosyllactose |

A number of embodiments of the invention have been described. Nevertheless, it can be understood that various modifications may be made without departing from the spirit and scope of the invention. Accordingly, other embodiments are within the scope of the following claims.

1. A method for:
controlling, ameliorating, lessoning or preventing the symptoms of or the mortality of a dysbiosis or an infection in an individual in need thereof,
wherein optionally the infection is a bacterial infection or a viral infection,
wherein optionally the dysbiosis causes or exacerbates a Failure to Thrive (FTT) of the individual, and optionally the dysbiosis is in an infant, a child, an expectant mother or a mother (material dysbiosis), and optionally the infant is between 0 and 36 months old,
and optionally the dysbiosis can be the presence of a pathogenic bacteria, optionally having a high level of pathogenic bacteria, or the dysbiosis can be caused by a high level of antibiotic resistance, or a metabolic balance that skews away from that of a healthy population, or an immunological state that skews away from that of a healthy population, or a loss of metabolic function associated with a healthy population, or an increase in bacteria associated with adverse events for a mother and her child,
modulating the microbiome of an individual,
wherein optionally the individual is a human, and optional the human is a human child or a human infant, and optionally the infant is between 0 and 36 months old,
and optionally the microbiome of the individual is modulated to positively affects the growth, thriving or health of the individual (or increases the ability of the individual to thrive), or to enhance the efficacy of a treatment in an individual in need thereof, wherein optionally the treatment is a drug treatment, or a treatment for cancer,
treating, ameliorating, lessoning the symptoms or severity of, or preventing, a disease or condition caused by a dysbiosis in an individual in need thereof,
wherein optionally the individual is a human, and optionally the human is a human child or a human infant, and optionally the infant is between 0 and 36 months old,
wherein optionally the disease or condition is a Failure to Thrive (FTT),
wherein optionally the dysbiosis treated or condition treated or ameliorated comprises dysbiosis caused or exacerbated by: premature birth, extended stay in the neonatal intensive care unit, drug or antibiotic treatment, drug or antibiotic treatment of the mother prior to birth, birth via cesarean section, formula feeding, and known dysbiosis of the mother,
treating, ameliorating, lessoning the symptoms or severity of, or preventing, an allergic reaction (optionally a food allergy), a dermatitis (optionally an atopic dermatitis), atopic eczema, allergic rhinitis (hayfever), gastroesophageal reflux disease (GERD), rhinosinusitis, obstructive sleep apnea, celiac disease, irritable bowel syndrome (IBS), Crohn's disease, rheumatoid arthritis, Sjögren syndrome and/or asthma,
treating, ameliorating, lessoning the symptoms or severity of, or preventing obesity or metabolic syndrome, non-alcoholic fatty liver (NAFL), or metabolic dysfunction-associated steatotic liver disease (MASLD),
treating, ameliorating, lessoning the symptoms or severity of, or preventing diabetes (optionally gestational diabetes, type 1 diabetes (T1D) or juvenile diabetes, or Type 2 diabetes (T2D) or adult-onset diabetes), acute or chronic hyperglycemia, and/or
the method comprising:
(a) administering or having administered to an individual in need thereof a composition or formulation comprising:
(i) at least two different species or genera (or types) of non-pathogenic bacteria (also called probiotics) and/or non-pathogenic bacterial spore, or
(ii) at least one non-pathogenic, live bacteria and/or non-pathogenic bacterial spore and at least one probiotic (also called a synbiotic, or combination of a probiotic and a prebiotic),
wherein each of the non-pathogenic bacteria comprise (or are in the form of) a plurality of non-pathogenic colony forming live bacteria, a plurality of non-pathogenic germinable bacterial spores, or a combination or mix thereof; or,
(b) (i) providing a composition or formulation comprising:
(1) at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable bacterial spores, or a combination thereof, or
(2) at least one non-pathogenic, live bacteria and/or non-pathogenic bacterial spore and at least one probiotic (also called a synbiotic, or combination of a probiotic and a prebiotic),
wherein optionally the at least two different species or genera (or types) of non-pathogenic bacteria of (b) (i) (1) or the at least one non-pathogenic, live bacteria and/or non-pathogenic bacterial spore of (b) (i) (2), is genetically engineered to comprise or express a new or heterologous trait or phenotype; and
(ii) administering or having administered to an individual in need thereof the composition or formulation;
wherein optionally the composition or formulation comprises one, or a or any combination or mix (or consortium) of: one (optionally, as in a synbiotic, or combination of one species and a probiotic, optionally a synbiotic combination as set forth in Table 8 or Table 32), or at least two different species or genera of non-pathogenic, live bacteria (or spore thereof if the bacteria is spore forming) as described Table 1 or Table 4, or live biotherapeutic (also called probiotic) compositions or combinations of bacteria as set forth in Table 2 or Table 30, or the at least one non-pathogenic, live bacteria and/or non-pathogenic bacterial spore and at least one probiotic (or synbiotic) comprises a combination as set forth in Table 8 or Table 32,
and optionally at least one of the bacteria in the synbiotic as provided herein, or in a combination, mix (or consortium) as provided herein, is a Bifidobacterium or a Bacillus species, optionally a Bifidobacterium infantis specie,
and optionally the different species or genera (or types) of non-pathogenic, live bacteria are present in approximately equal amounts, or each of the different species or genera (or types) of non-pathogenic, live bacteria or non-pathogenic germinable bacterial spores represent at least about 1%, 5%, 10%, 20%, 30%, 40%, or 50% or more, or between about 1% and 75%, or between about 0.5 and 99%, of the total amount of non-pathogenic, live bacteria and non-pathogenic germinable bacterial spores in the formulation,
and optionally only or substantially only non-pathogenic, live bacteria are present in the formulation, or only or substantially only non-pathogenic germinable bacterial spores are present in the formulation, or approximately equal amounts of non-pathogenic, live bacteria and non-pathogenic germinable bacterial spores are present in the formulation.

2. The method of claim 1, further comprising administering or having administered one or any one of: a treatment, a prebiotic, synbiotic (or combination prebiotic and probiotic, optionally, a synbiotic as set forth in Table 8 or Table 32), a metabolite or a drug, optionally an anti-viral or anti-bacterial treatment or drug; an immune checkpoint inhibitor; a Chimeric Antigen Receptor (CAR) T-cell therapy (CAR-T), or an immunotherapy (optionally, an immune-enhancing therapy); or a combination thereof,
and optionally the method comprises administration of: an antimicrobial drug, optionally an antiviral, antibacterial, antifungal or antimalarial, drug, and optionally the antimicrobial (optionally antiviral) drug comprises one or any one of: lopinavir; ritonavir; oseltamivir (optionally, TAMIFLU™); lopinavir combined (formulated) with ritonavir, or KALETRA™; chloroquine phosphate (optionally, RESOCHIN™), chloroquine diphosphate, hydroxychloroquine (optionally, PLAQUENIL™) or oral chloroquine (optionally, ARALEN™); remdesivir (optionally, GS-5734™, Gilead Sciences); nevirapine, efavirenz, emtricitabine, tenofovir (or the combination efavirenz with emtricitabine and tenofovir, or ATRIPLA™); amprenavir (optionally, AGENERASE™); nelfinavir (optionally, VIRACEPT™); a thiazolide class drug, optionally nitazoxanide (or ALINIA™, NIZONIDE™) or tizoxanide (or 2-Hydroxy-N-(5-nitro-2-thiazolyl)benzamide); plitidepsin (also known as dehydrodidemnin B), or APLIDIN™ (PharmaMar, S.A.); an inhibitor or S-phase kinase-associated protein 2 (SKP2), or dioscin, or niclosamide, or NICLOCIDE™, FENASAL™, or PHENASAL™; ribavirin; an interferon such as interferon alpha, interferon beta, interferon type I, interferon type II and/or interferon type III, or a combination of ribavirin and interferon beta, or a combination of lopinavir and ritonavir and interferon-beta-1b; abacavir, actemra, acyclovir optionally, (ACICLOVIR™), adefovir, amantadine, ampligen, amprenavir (optionally, AGENERASE™), aprepitant, atazanavir, balavir, baloxavir marboxil (XOFLUZA™), bepotastine, bevirimat, bictegravir, biktarvy, brilacidin, cidofovir, caspofungin, lamivudine and zidovudine (optionally, COMBVIR™), cobicstat, colisitin, cocaine, danoprevir or danoprevir and ritonavir (optionally, GANOVO™) darunavir (or darunavir and cobicstat, optionally, PREZCOBIX™), delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, ibacitabine, icatibant, idoxuridine, ifenprodil, imiquimod, imunovir, indinavir, inosine, lamivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (optionally, BCX4430 by Biocryst, IMMUCILLIN-A™), remdesivir (optionally, GS-5734™, Gilead Sciences), cytarabine, gemcitabine, emtricitabine, zalcitabine, stavudine, telbivudine, zidovudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, ribavirin, rilpivirine, rimantadine, ritonavir, saquinavir, sofosbuvir, telaprevir, tegobuv, tenofovir alafenamide, tenofovir disoproxil, tenofovir, tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™) or zidovudine; a serine protease inhibitor, optionally camostat; an anti-PD-1 checkpoint inhibitor, optionally camrelizumab; a compound or antibody capable of binding complement factor C5 and blocking membrane attack complex formation, optionally eculizumab; a cathepsin inhibitor, optionally a cathepsin K, B or L inhibitor, optionally relacatib; thalidomide, or thalidomide and glucocorticoid (optionally low-dose glucocorticoid), or and thalidomide and celecoxib; an antibacterial antibiotic or a macrolide drug, wherein optionally the macrolide drug comprises azithromycin (optionally, ZITHROMAX™, or AZITHROCIN™), clarithromycin (optionally, BIAXIN™), erythromycin (optionally, ERYTHROCIN™), or fidaxomicin (optionally, DIFICID™ or DIFICLIR™), troleandomycin (optionally, TEKMISIN™), tylosin (optionally, TYLOCINE™ or TYLAN™), solithromycin (optionally, SOLITHERA™), oleandomycin (or SIGMAMYCINE™), midecamycin, roxithromycin, kitasamycin or turimycin, josamycin, carbomycin or magnamycin, and/or spiramycin; opaganib or YELIVA™; an anti-interleukin-6 antibody (e.g., tocilizumab or tocilizumab and favipiravir, optionally, ACTEMRA™); sarilumab (optionally, KEVZARA™); umifenovir (optionally, ARBIDOL™); colchicine, or COLCRYS™, MITIGARE™; a corticosteroid class drug such as budesonide (or RHINOCORT™ or PULMICORT™), prednisolone (or ORAPRED™), methyl-prednisolone, prednisone (or DELTASONE™ or ORASONE™) or hydrocortisone (or CORTEFT); an anti-androgen drug, or bicalutamide; a hydrocortisone or cortisol (or CORTEF™, SOLUCORTEF™), or hydrocortisone sodium succinate or hydrocortisone acetate or dexamethasome (or DEXTENZA™, OZURDEX™, NEOFORDEX™); famotidine, or PEPCID™; an antihistamine class drug such as azelastine, or ASTELIN™, OPTIVAR™, ALLERGODIL™, brompheniramine, fexofenadine or ALLEGRA™, pheniramine or AVIL™, or chlorpheniramine; a dendrimer, or an astodrimer sodium (Starpharma, Melbourne, Australia); a selective serotonin reuptake inhibitor (SSRI) class drug, optionally fluvoxamine, or LUVOX™, FAVERIN™, FLUVOXIN™; a nicotinic antagonist, a dopamine agonist or a noncompetitive N-Methyl-d-aspartic acid or N-Methyl-d-aspartate (NMDA) antagonist; an immunosuppressive drug, or tocilizumab or atlizumab, or ACTEMRA™, or ROACTEMRA™, or a calcineurin inhibitor (CNI), or ciclosporin or cyclosporine or cyclosporin); or, any two, three or more or combination thereof;
and optionally the anti-viral treatment or drug, the immune checkpoint inhibitor, the Chimeric Antigen Receptor (CAR) T-cell therapy (CAR-T) or the immunotherapy, or the combination thereof, is administered before, during (concurrently with) and/or after administration the formulation.

3. The method of claim 1, wherein:
(a) the composition or formulation comprises an inner core surrounded by an outer layer of polymeric material enveloping the inner core, wherein the non-pathogenic bacteria or the non-pathogenic germinable bacterial spores are substantially in the inner core, and optionally the polymeric material comprises a natural polymeric material;
(b) the composition or formulation is formulated or manufactured as or in: a nano-suspension delivery system; an encochleated formulation; or, as a multilayer crystalline, spiral structure with no internal aqueous space;
the composition or formulation is formulated or manufactured as a delayed or gradual enteric release composition or formulation, and optionally the formulation comprises a gastro-resistant coating designed to dissolve at a pH of 7 in the terminal ileum, optionally an active ingredient is coated with an acrylic based resin or equivalent, optionally a poly(meth)acrylate, optionally a methacrylic acid copolymer B, NF, optionally EUDRAGIT S™ (Evonik Industries AG, Essen, Germany), which dissolves at pH 7 or greater, optionally comprises a multimatrix (MMX) formulation, and optionally manufactured as enteric coated to bypass the acid of the stomach and bile of the duodenum.

4. The method of claim claim 1, wherein the plurality of non-pathogenic colony forming live bacteria are substantially dormant colony forming live bacteria, or the plurality of non-pathogenic colony forming live bacteria or the plurality of non-pathogenic germinable bacterial spores are lyophilized,
wherein optionally the dormant colony forming live bacteria comprise live vegetative bacterial cells that have been rendered dormant by lyophilization or freeze drying.

5. The method of claim 1,
wherein the formulation comprises at least about 1×10⁴ colony forming units (CFUs), or between about 1×10¹ and 1×10¹³ CFUs, 1×10² and 1×10¹⁰ CFUs, 1×10² and 1×10⁸ CFUs, 1×10³ and 1×10⁷ CFUs, or 1×10⁴ and 1×10⁶ CFUs, of non-pathogenic live bacteria and/or non-pathogenic germinable bacterial spores.

6. The method of claim 1,
wherein the formulation comprises at least one (optionally as in a synbiotic, or combination of one species and a probiotic, optionally a synbiotic combination as set forth in Table 8 or Table 32), or (or any one, several, or all of) non-pathogenic bacteria or spore of the family or genus (or class): Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), or a combination thereof.

7. The method of claim 1,
wherein the formulation comprises water, sterile water, saline, sterile saline, a pharmaceutically acceptable preservative, a carrier, a buffer, a diluent, an adjuvant or a combination thereof.

8. The method of claim 1,
wherein the composition or formulation is administered orally or rectally, or is formulated and/or administered as a freeze-dried composition, a liposome, a liquid, a food, a gel, a supplement, a gummy, a candy, an ice, a lozenge, a tablet, pill or capsule, or a suppository or as an enema, or the formulation is administered as an or is in a form for aerosol, topical, sublingual, oral, intra-rectal or intra-colonic administration.

9. The method of claim 1,
wherein the composition or formulation comprises or is mixed into: milk (optionally, human milk, cow's milk or soy protein, and optionally fortified with vitamins, minerals, and other nutrients), infant formula, soy-based formulas, amino acid-based formulas, hydrolyzed infant formula (optionally made from cow's milk or soy protein that has been broken down into smaller proteins that are easier for infants to digest), or supplemental (harvested) human mother's milk.

10. The method of claim 1,
wherein the composition or formulation is administered to the individual in need thereof in one, two, three, or four or more doses, and wherein the one, two, three, or four or more doses are administered on a daily basis (optionally once a day, bid or tid), every other day, every third day, or about once a week, and optionally the two, three, or four or more doses are administered at least a week apart (or dosages are separated by about a week).

11. The method of claim 1,
wherein the composition or formulation further comprises a prebiotic, a synbiotic (or combination prebiotic and probiotic, optionally, a synbiotic as set forth in Table 8 or Table 32), a nutrient, a metabolite or a drug, and optionally the drug comprises an antibiotic, or the method further comprises administration of a prebiotic, a synbiotic (or combination prebiotic and probiotic, optionally, a synbiotic as set forth in Table 8 or Table 32), a nutrient, a metabolite or a drug, and optionally at least one dose of the prebiotic, the synbiotic, the nutrient, the metabolite or the drug is administered before a first administration of the formulation, mix or consortia of bacteria, optionally at least one dose of the drug (or antibiotic), the prebiotic, the synbiotic, the nutrient, or the metabolite is administered one day or two days, or more, before a first administration of the formulation.

12. The method of claim 1,
wherein the drug (or combination prebiotic and probiotic, optionally, a synbiotic as set forth in Table 8 or Table 32), prebiotic, the synbiotic, a metabolite, a metabolic precursor, or a nutrient is administered by: aerosol, spray, intravenous (IV) injection, intramuscular (IM) injection, intratumoral injection or subcutaneous injection; or, is administered orally or by suppository.

13. A composition or formulation or a pharmaceutical composition comprising:
(a) a combination, mix or consortia of microbes as set forth in Table 1 or Table 4, or live biotherapeutic compositions or combinations of bacteria as set forth in Table 2 or Table 30;
(b) a combination, mix or consortia of microbes as used in any of the preceding claims, or as used in a method of claim 1; and/or
(c) at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable non-pathogenic bacterial spores, or a combination thereof, and the formulation comprises at least one (or any one, several, or all of) non-pathogenic bacteria or spore of the family or genus (or class).

14. The composition or formulation or a pharmaceutical composition of claim 13, wherein the composition or formulation or a pharmaceutical composition comprises at least one (optionally, as in a synbiotic, or combination of one species and a probiotic, optionally a synbiotic combination as set forth in Table 8 or Table 32), or a mix or consortia of bacteria, having at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable non-pathogenic bacterial spores, or a combination thereof, and the formulation comprises at least one (or any one, several, or all of) non-pathogenic bacteria or spore of the family or genus (or class): Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), or a combination thereof.

15. The composition or formulation or a pharmaceutical composition of claim 13, wherein
(a) the composition or formulation or a pharmaceutical composition comprises an inner core surrounded by an outer layer of polymeric material enveloping the inner core, wherein the non-pathogenic bacteria or the non-pathogenic germinable bacterial spores are substantially in the inner core, and optionally the polymeric material comprises a natural polymeric material,
(b) the composition or formulation is formulated or manufactured as or in: a nano-suspension delivery system; an encochleated formulation; or, as a multilayer crystalline, spiral structure with no internal aqueous space;
(c) the composition or formulation is formulated or manufactured as a delayed or gradual enteric release composition or formulation, and optionally the formulation comprises a gastro-resistant coating designed to dissolve at a pH of 7 in the terminal ileum, optionally an active ingredient is coated with an acrylic based resin or equivalent, optionally a poly(meth)acrylate, optionally a methacrylic acid copolymer B, NF, optionally EUDRAGIT S™ (Evonik Industries AG, Essen, Germany), which dissolves at pH 7 or greater, optionally comprises a multimatrix (MMX) formulation, and optionally manufactured as enteric coated to bypass the acid of the stomach and bile of the duodenum.

16. The composition or formulation or a pharmaceutical composition of claim 13, wherein the composition or formulation comprises water, sterile water, saline, sterile saline, a pharmaceutically acceptable preservative, a carrier, a buffer, a diluent, an adjuvant or a combination thereof.

17. The composition or formulation or a pharmaceutical composition of claim 13,
wherein the composition or formulation is formulated for administration orally or rectally, or is formulated for administration as a freeze-dried composition, a liposome, a liquid, a food, a gel, a supplement, a gummy, a candy, an ice, a lozenge, a tablet, pill or capsule, or a suppository or as an enema, or the formulation is formulated for administration as an or is in a form for aerosol, topical, sublingual, oral, intra-rectal or intra-colonic administration.

18. The composition or formulation or a pharmaceutical composition of claim 13,
wherein the composition or formulation comprises or is mixed into: milk (optionally, human milk, cow's milk or soy protein, and optionally fortified with vitamins, minerals, and other nutrients), infant formula, soy-based formulas, amino acid-based formulas, hydrolyzed infant formula (optionally made from cow's milk or soy protein that has been broken down into smaller proteins that are easier for infants to digest), or supplemental (harvested) human mother's milk.

19. The composition or formulation or a pharmaceutical composition of claim 13,
wherein the composition or formulation is formulated for administration to the individual in need thereof in one, two, three, or four or more doses, and wherein the one, two, three, or four or more doses are formulated for administration on a daily basis (optionally once a day, bid or tid), every other day, every third day, or about once a week, and optionally the two, three, or four or more doses are formulated for administration at least a week apart (or dosages are separated by about a week).

20. The composition or formulation or a pharmaceutical composition of claim 13,
wherein:
(a) the composition or formulation further comprises a prebiotic, a nutrient, a metabolite or a drug, and optionally the drug comprises an antibiotic, and optionally the drug comprises an antibiotic, and optionally at least one dose of the prebiotic, nutrient, metabolite or drug is administered before a first administration of the formulation, mix or consortia of bacteria, optionally at least one dose of the antibiotic is administered one day or two days, or more, before a first administration of the formulation; or
(b) the drug, prebiotic, a metabolite, a metabolic precursor, or a nutrient is formulated for administration by: aerosol, spray, intravenous (IV) injection, intramuscular (IM) injection, intratumoral injection or subcutaneous injection; or, is administered orally or by suppository.

21. (canceled)


SEQUENCE LISTING (300 sequences)

SEQ ID NO: 1 - protein, 150 aa, Bifidobacterium longum
MSGTRQYHRLSAETLDTLLRLISEDELTPKQIAERAGVPRQKVYEYRKKLKDRRKSAPLTDISTLVIHQRVVFRPDATIENPEDVNGPSFIDPDSGFDCSRCGQSMSRDWFTIQGNLIKPDFGYCPGCGGVATPYRDDTINPDAREAGDE

SEQ ID NO: 2 - protein, 88 aa, Bifidobacterium longum
MKFHRISPCPRCGGKVRAKWERDEVLALPEYTFFIVMFRCTACGLSLDGGCSRKPAPYQLQRSIVVWNRVCNGDKCFTLLYKILAGGR

SEQ ID NO: 3 - protein, 164 aa, Bifidobacterium longum
MSATILDPACGGRMFWFDKHDPRVLFGDCRDESWELCDGRRFDVKPDQLMDYRHLPFPDDSFRLVVLDPPHIRHGGRTSYMVRKYGLLDEHGWPDDLTSMFAECFRVLEPSGILIFKWNETQIPVSQVLACTPQHPLFGNKQPKQTGTHWIVFMKEALDEIPQD

SEQ ID NO: 4 - protein, 105 aa, Bifidobacterium longum
MNNLIHCDMCGYLMTKRWSETIDGKTYCRDCVPKKRLIDSGEPTEFDDTDEIVCPYCGHRYEDSYECGGNDEYFEEECEDCGREFNVTRIIDISYDTKPKEATEE

SEQ ID NO: 5 - protein, 119 aa, Bifidobacterium longum
MSEPEQNWPESEPDKSKPEMTQEELQRQLLETQKRLVELQEQQIRKKDSQTDNHDTGIGKSVVSILIAAVIIAVVGFVIHDYTKTKEVNEHASDNISDALNLGSGSPTDIFLSSLPMKP

SEQ ID NO: 6 - protein, 206 aa, Bifidobacterium longum
MSSPVYQPMPPAGMQPSQPVSHRRKKGITIPVPVFVLLIIGIIVALFVGMLFGVGAMSEEVNSAQQSSYAWEDKYKAAKVQLNGYVSAPSVAPDTMGACGVLFDGNQSLIDDVAAVSGYFRDPANHDAVQAFSGASLAVKTINEAFPKADPDMKASLAALNAPMLKIVYATQNLGYADAQYDSLQVLSDLNSVMESCVAVGYTAKQ

SEQ ID NO: 7 - protein, 68 aa, Bifidobacterium longum
MVMGSAENHPQSRYARQLPPAGASTGKEDMRATLETVSCGELTAVYRKDSDTGIVELASWIVDASSVL

SEQ ID NO: 8 - protein, 692 aa, Bifidobacterium longum
MTFAYGDDRPVAVQSVQMNDVKTRFDRTTPIAEIITTGDGHVPAGNRLTHTGIGLALRYRGHRATVEGTRHTLAVTLGDDVRGIEALVRYEVDERVAMVRTDVTVTNTGAAPLLVDFVTSWSSAFGAPEGVVPDARAWDLFEGRSDWLAEGRWSRRPVNDLLPVISQELTGVDPRQGHQVVSSGTWSTGMHLPLAVLESKTFGLAWLFQVEHNGAWRWDVEDDTVDGGIALSGPTNENHGWCRDLKPGESFTTVPASFTLAGDFDAAVRRVTDYRRVMRAPHPDHAVTRTVFNDYMNTINGDPTTEKELPLIKAAGETGVEIFVIDCGWYDDSGDWWPSVGEWMPSKTRFPGEKGIVEVVDAIKAAGMVPGIWLEPEVVGVDSPVAKRLPDSAFFQRRGRRVMEHSRYLLDFRDPVARAHVDAVVDRLVTEYGIGYFKFDYNVSPGSGTDYDADAPGDGLLGHNRAYSAWIESLHRRYPDLILENCSSGGMREDFAQTGRFQVQSTSDQQDWRLYPVIAAAAPMMVLPEQAASWAYPQSDMTDEETAFNINTTFLGRFFLSGYLNRMDAGQLDIVRQGVKAYRRHVQPVIGQSVPFWPLGLPGWDDPLLAYGLDCGDTALVTVWNRGGEGGDVRLDLRRWRGRAGAVAAVYPTEGFESWPVHWDAGRGELVVRVPAGVYVSRTFEISFAPRS

SEQ ID NO: 9 - protein, 332 aa, Bifidobacterium longum
MASRVTLNDIAAAANVSKATVSKALNDTGQLSARTRRVVLAAADRLGYVRPRPMTRSRSGLIGLVSADLDGRFATPALTGAENTLGAASHAVLLTNSRGDPKLERAHIDQLAARGVDGLLMLGGETDARPPVRPSTALDIPIVYTYAPSTDPNDCSVTCDNVAAGAAAIDHLLSRGRRRIAIIAGPEYYQATKDRLVGAARAMKVAGLRLAVPTRYGNWHESWGRTATNLILESGVPIDGIYCLNDMLARGAIETLMDCGLDVPRDVAVIGHDNWAVTATEGPVPITSFDSNLQEIGRRSARLLLDIIRGNPRHGTMLIGCSLVVRQSTVAR

SEQ ID NO: 10 - protein, 417 aa, Bifidobacterium longum
MNVHFSGTAKRIVASIAAVAALGSMAACGSNGASNGGSDDELTVSYWDDEQDSIKEFIKQNPDIKVKEIRVPGDDYNTKLNQMIVGNTAPDVMLVQEADYVRFAQNGVLEKLDDQLSDLGIDKDDFQPAVKGITNQVDGYYGFPQGFATEIMYYNKDLFDAAGVEYPTNDWTWDDYTAAAEKLTDASTGQYGSDSPTFNGVWYSLIGAAGDDVVKDGKLSFGNGLKETLEFQKNLVDNKWQPEPASGSKVSDMFAAGKAAMTMGGTWLVSTYKDADFNWDIATIPTPEGGRKYNSLHTSFWTISKNSKHKDAAKKLVKFLMSKEGQKAMSQQLGNAPAFQSMMSDGYYRVEGKHGPSNWDVLTQSTEEARLGYTMVSSTPTFDLYDQFNAYVLGQTSLDEVTGAQVDKANKEITDAQ

SEQ ID NO: 11 - protein, 288 aa, Bifidobacterium longum
MHSDTGAAWGFLSPWIIGFLVFSAFPLAFSFYLSLTKWNLMGDPQFVGLQNYKDMLSGQGELGQTLLATFIFTVINVAVSILFSLLLAVLLNFKVRFKGLFQFFYYVPTIMPSVVMAGCLVLMFNPQLGIINYVLKCIGVENPPNWGGSQTFVWVMVAVASIFTFQTGQQMLVFSAALKDVPQELYEAAALDGAGAWKRFIHITIPGIAPMMLFNVVSCTVNSFNSAFSLLYPLTGGGPGNATKVIGLLIYDKAFKSFNMGQASALSVILFIIVGLISILQFRLMDRK

SEQ ID NO: 12 - protein, 281 aa, Bifidobacterium longum
MYHTPKYVKVLQYIAMILMALFMFFPIYWIFVNSLKTINGISAWPPEFFPSDPQWGNYIEVLKNPNTLLYLRNTLILVVFNTLGTLLTSALVAYPLARMHFKGRGVIFGIILATMMVPSAALVIPQYLLFRSFGMLDSFWPLILPSFFAQPYNVFLFRQFFVSIPESIDEAAMLDGCSRWQAYWRVIVPLGKPIFITVGIMSASFWWNELFSPLVYINSEDLKPLTLGVLTSFVQTSAGASKTMWNLQMAFSMLMIIPPALMYIFCSKYITEGIKTSGMKD

SEQ ID NO: 13 - protein, 658 aa, Bifidobacterium longum
MTDQTTSPKVTITSRFWHRYRTMTVDNALPYQWRALNDEVPVDVPEGAAWGENGSQFSHSLRNLRIAAGRESGAFSGQPFQDTDVSKWLEAASYTLRMRDEGFDIDDIEAKVDEAIGLFEDLQDEDGYLDTKFEIDLPADQRFKGLRWSHELYTMGHFIEAAVAHYESTGSPRALDIAERAADCIDRHFGDAAGQIHGPDGHPEIELALARLYEVTGERRWLDLAAWFIRVRGIDPEFYDEQDKAGGPQFYTDMHMPLKYFVADEPILDKAKAEGHAVRLLYLAVAVAKVGRLLEDRKMLDTAERLWRNIVDHRMYVTGAVGSTQVGEAFSFDDDLPNDLVYGETCASVAMLFYGKALMEIRPRGSVADVMELELFNGMLSGIQLDGTRYFYVNPLEADPAASAGNPTRHHVLTRRAGWFDCACCPANLVRLITSLDRYLYTESGDTIYAHQFIANRAEFADGLTVEQTQAGEEYPWSGDITFHVANPNRLNKRLAVRIPAWSPRWTLEVNGNPVNLKAADGFVSIDVSGETTEIHLVLDMAVRKVRASLDVRCDVGRLAVARGPIVFCMEQCDNEGPLWLDGMSVDAEVEERYDADLLDGVEVLTVEGRRFETQRTGQYYTAEAPLAEHEQQLTLIPYYAWCNRAEGQMQVWVRETR

SEQ ID NO: 14 - protein, 1069 aa, Bifidobacterium longum
MRSPKPLAALAAAAVLGGGLAFGAAVPAYAIENPTVTVHLDQKGCTENSNNTDCQIFHGSTGFLYGLTDDGISSDTTLAGLNLDEDSVHVGKSPNGVQHPNGDVMNTTDQWKRNGGGEIQVYMKEAYEGFPYAAYGDGSINSYVEKVKTMVQTFNDKYPEYKDDIVWIPFNEPDISDQNYYNLTNYSSQYDSVRTRFFEDWDKVVSAIREVYPAARIGGPNNSGWNNTFYRDFFNHAKANGTVPDVVTWHELGSGFGSYLSNFQQWKALEKTILDGYEYPEGTALQPGQNIKVSINEYAWKDNNGNAIEQVKPGRLLQYIARFEKTGAQGALPYWYPAGDLDWLVTHNNQVTGSYWLYYWYGLMEGDLLKVDLSDENGKPQVLASYDASSNQTQILLGGANEAEFNTTLNLEALQDKYPNGAHVTVYATDFTAPADMDNVAESVPAASDGPYIVVDQDLAIADGHASLALNNLKGDSAYYAVVTPATAQGAVAKDTVEAEYARRNGTATVNYGNATGYSGTGYVSGTDEAASSDFFVNSTKDGYSEVTLRYSAPKVEGQSATRNVTLKINPSENTARNDVQDVTLNLPETKDANTWQTAKIRVYLPLGLNQFTVEGFGAQGVLIDSLGINSADDSSVTRYEAEASSNTFNGSANVSNNNNASNQKIVGNVGNGANNWFQFNNVTVPADGNYTVTIGYSQWEYTDNNTWQIVNRWADMSVNGGEAKHLVFANTRSWNNIWTTSVRVNLKKGTNTLKFSNSDGSGSASKDGKPSGWAPNFDYIQVAPTVDGGATYTTADGGEVKAITSLTAKADGLKDGVLTLTEGDSVDVDVNIDPIDSTDPTLTWTSSDASVATVEDTENAGKAKTRSVKLTDTRRIHALKAGETTITVVPTVNAADGVAATFKVVVNAEDTPTPDVDKSKLEAAVDAANKLDESAYTADSWKAFAEVLASAEGVLQNTDATQSDVDSAVELLADAQGKLVKADSEEGDNPSGGETGDNPDDQTGDQDGDNAGSGEGDASTTDSDDKTDSGSHRMPSTGSAIAVVAAAAGVLVLAAGGLLIVRHRRAKH

SEQ ID NO: 15 - protein, 206 aa, Bifidobacterium longum
MNSMAQHTSHASINDPEVLPTPETVVLDANIFPSTWLTDLFLSLAEHTGLLEIIYSDTILEESRRAMIDDLGFAAHWVDRYLSSIQMGFPYSRVVPDPDTMHRIVLPDPDDSHVVAAAVAAGASTIVTYNLADFPESELAPYAVKALHPDRVLTKILCNHTKPVADVLREIVSSKTRPPRTMPEELEQLHRLGLHSFVEAAAPHVL

SEQ ID NO: 16 - protein, 131 aa, Bifidobacterium longum
MTLTDSIPLAEVRPDSPYLAYGDGTALPLDADELRLIINALAEQDEMITTGEAAKILHVSPRTVARIVDAGEIPSVRYGRLGNRMVSKRDVLAFLDKSSQRTSEGLDSMRTAAYQGGLDELDGAAYIARFN

SEQ ID NO: 17 - protein, 340 aa, Bifidobacterium longum
MANKTAANRKPSMGDVAEAVGVSKTTISRYLHGEFGCMSPETKARIEAVISELGYRPNKMAQGLKATVSHLVGVTIADIGNPFSSLLLKGIQQECRARDVQLLVSDSNNQASFERANIESLLDAQVDGLIVNTVGNNDDWLSEYCARRNHKPMVMLDRIVQPIVCDCVATDNHGAVFEMLDYLVDRGFDYVVLVTRPSDGISTRTMRREAVEQYFLDRGLRGEVLVYREDAADLADCLISTIAAHGGERICLFANNDETMHDVLEALPASPDGHVGVCAFANERWAKYSGIGITCLDQNPVEMGRAAARVLLARIYDGYDGPYELKEMPARLCAFASTEA

SEQ ID NO: 18 - protein, 49 aa, Bifidobacterium longum
MDMSMDTHMDTSMDIHMDTSMDIHMEMPMDIHMEMPIKSKVSKLDGSCT

SEQ ID NO: 19 - protein, 441 aa, Bifidobacterium longum
MITWIMGILLIASFACFVIYAMRGGNLTIGFFILTIVWTLVYFLGIPFGVGEPFDVIKETFAEPALTYGPTIVQIVCGAWFGRVLVDTGIAASISYRTAKVGEKNPVLATICVAFVTCLIFTSAYGVGSAIAIGVILFPIMGRLGVPKRVAVPVFTLSIGAAMWINSVMFVQFAAFYQGYESPDGQNIEWGTHYLSFGIPAMLVQMAAVVIFILINAKAIENGTPYEVGDPNERPASVPEVPIWTYVLPVVPVALSIVFQWDSVPALFLAVIIAFFGTGYMKTYKGFVSILNSTAKTAIGDIGSLIIMLLVLRFFQHAAVTVMADFGPALTAVVPNNEFILALAVCILAPLALFRGPLELYGAGSAVISILMGMGVFNSWFLFAMLVVPSMTCISSCVTQSWNMWAVEYNELEPKTFLKNGVPVYWLCTFPIMGLASLLLF

SEQ ID NO: 20 - protein, 575 aa, Bifidobacterium longum
MTTIGINTLVYMTELANGTPQSDLLPIIASHGITLAEVRREYIASDTEFDLIAAAAQANGLDLFYSVPESLTIDGAVNPGFAGFLDEARRMGVTNVKFNQGDVKDVAKSVIDDIDAAAASYGVTLTIENDQTPENGTLDCTVASLRHIKELGGNIGYTFDLGNWFWRGENAGEAFAQLLPSITVFHLKNVNGAATREELATTMLEDGVIDWKAMLPQLDASVPVFLEFPIPADGVAAQVAQVREVVGQKTPIMSEVMTIGEPMVNLIADSAETFMEARTLPREMAGAEFNVAIGVKRQGHSISYVTTLGNDWQGDLIVDYMNNIDIDTTNIRRVDGAATGYQLKVRSSDGEPKVIYFRAGSAASQTAPDIVDGIDFDGLKILHVTGIFSALTDNTYATVMRLVDAAKAHGVTVTFDPNPRPTLWNSEEQMIEATNRIAAKCDVFMPGLNEGQLFSGLTDPRAIADYYLDMGVRQVVIKLGAEGSALFERDESGARRETVVPSFEVDVVDTVGAGDGFASGVITSLLEGLDDEHLLERANAVGAIQVTSVSDSEGLPTAGELAEFIANTPRKEVSL

SEQ ID NO: 21 - protein, 270 aa, Bifidobacterium longum
MPGSQISFALYSVYLKSLLQNNNFGYTLIMVLDINQRMLTTQETAQRLGVSTARVSALVKNDDLQSIAVGKTRLITALSVAQHQHQDSRPGRLFAPHIALGTLYLLSGVEATWLNAKERYRIRNYLKTVKADRLARLCARRATTMDMWCPSDAIPMLVSDIAISAATGELAVAFGLARTDKVEGYIASDSLEYLVDHYDLETDLAPSTVRLHISELIDKQTSSMPVGICAVDLTESFDIRERNAGLVMLEHLLTGFRSNSAREKVGNAIN

SEQ ID NO: 22 - protein, 242 aa, Bifidobacterium longum
MQSINIQCISHPWSTVHDLSIADPTGNWLLAGGLMVQVHAILGGLPIRPTQDADLLMDLISQPNEANRVRHLLSSFGFTIHPGTLTGYTTRMVSVNGSIVDVLVADHLPNHLAKESTYSGFPILPMPGGAQAIERSMTVEIDSGTDRFPLRVPDLLGATMLKSAAWETDKARDRSRHLSDAALLLSLMREPQIELARLHSKTDRKRIRTLTQHLGRDAEAWDFLNLDHRRYGLRALQILAQY

SEQ ID NO: 23 - protein, 328 aa, Bifidobacterium longum
MEKIVDTQEPISFLELLGFSDSTLQEYTIRLNQSNENWFDVTNTYYSNNQQLMDWVFTKTWANDAKTKGKIPTNKVLQFIQLYKDEKPTIHWLFIGGFEIIGESLQANGNTLYEYKTIPQFKTLSGRSVVKYRKYRGDTQLINDLRNNDRRKRFISNLALDKITQSPISAQPFPGYLNIRLSFPELAAAVKNDEWRSALNSINAVYLQTDTLTGWHYVGSAYSRKGGTHGLLSRWEEYVSGDHTGENKQLQQLVKSKGKEYIEDNFQYSILEIFDSRISIKDIIRREHWWMTTLSSVYDPEGNPENCHGYNTKLEWNRTAEGKPADKA

SEQ ID NO: 24 - protein, 217 aa, Bifidobacterium longum
MKLQVAIDRVDIPRVEAIIDQVAGEADIIEIGTSLTKEYGLRALAPVCERLAARPAGETGRKAVLLGDIKTCDEGKYEFDLGFDCGFAYLTVMGSSSLGTLEVCAASAADHGGEMMIDLLECDESRIELISGFPDAVYCLHTSVDSGATADPVGQVRAFKARFPQITRIGIAGGIKHDQLAGLAAEGVEIAIMGSAITKAGDIAAACRACADACHSA

SEQ ID NO: 25 - protein, 307 aa, Bifidobacterium longum
MPSFVVGAYASLPQGREAQEAYYDLLGGQPWIDGTEIPFPGDLAETADRIWLAGQLPRHWKNNTVTAIPGTMQHVWKDPNFGLASPDEDGRRFALAFFKQLRDALADFAQWRGSQDVKFVEIHTAPTRIASRDAMTASLQALAQLDWSGAKLVIEHCDAYVEGRKPEKGFLPIEDEIALCRESGIGLTVNWGRSVVEGRKVQTAVEHIEAAAGAGVLAGLMFSGSGPEETQYGYGWIDGHLPMNPDEPTSLMDAAAIGVAVKAAGEQGEPLAYLGAKVCVPKDATAEERLGYLARIHDAVLAGRAGA

SEQ ID NO: 26 - protein, 335 aa, Bifidobacterium longum
MSGVAAGAATYLAFDIGGTKIASGFVTLPDENSAHADCGRKPRVEAQCEIPTEASRGGDDIRERLTAFASRQLARARDEGAVIRGIGIAAAGVPDSRTGVIVSATDILPGWRGQRIYDAFAKVTDLPVHMVGDVGAHGLGEAGYGAGRGHGIVLSIGVGTGIGGAIVVDGTLFSGAHGVAGHAGHVPSGLGRGFLCSCGTREGHIEPVASGTGLKDLYNARCDAEEGEPVADGSQVAARAAAGEPLAVGVIEDSARALGECIGGMGNLIDPDVIVVSGSVVKAGPLWWNALRAGFEDSALQLVRSAPLVEGELGGAAPLIGAAVAVRRHVAQGRP

SEQ ID NO: 27 - protein, 222 aa, Bifidobacterium longum
MHQVIEKIRGGLVVSCQAYPGEPLRHPETMAQMAMAAVEGGAVGIRCQGLADIAAIKGQVKVPVIGIWKEGDEGVYITPTLRHARCCAAAGADIVAIDATGRPRPDGLSYADTVHALHDEGVITMADCGSFADAERAVEAGTDIISTTLSGYTGERLKTDGPDFELLERMVKAFPDMPVLCEGRIHTPDQLHRVMECGAWAAVVGTAITHPTTITRWFAAKL

SEQ ID NO: 28 - protein, 519 aa, Bifidobacterium longum
MIKRNGKFKAAVAAAVASLMLLSGCGGGTQTAAKTGTAVADTITAQVAYASRDFSPSTTSGALPMAANWHVTEPLYALDYSTYEPYAALAKGDPEKVSDTEYVVTLRDGAKFSDGTAVTANDVVSSYQRTTATGSLYISMLDFIDSVEAKSDTQVTFKLKKAFPLFKQRLALIQIVPSSMSDADLKDKPIGSGPWKYAEITDQQVKFERNDLYNGSYPAQAKNMVWNVTVDDTARVTAMQGGKTDIMEMVPAQALQTLQSSGSELKTAQGFNLPFLMFNTKKKPFDDKRVRQAVFYAIDVDNLISNQMSGQAEAATSFLPKDSQYYHQAKNVYTKDTAKAKELLAEAGVTTPISFTLYTTDHSWITQLAPQIKNDLAEIGMNVDIQSMKSSALYPSITDKDDADYSMVLAPGDPSVFGNDPDLLMNWWYGDNAWTKQRSFWKGSDGYNQLHELMDKATAASSDSERQKYWNQCFDLLSEELPLYPLFHRKTTTAVRKGAFSSWEAIGSTGINLVKAKLN

SEQ ID NO: 29 - protein, 47 aa, Bifidobacterium longum
MALGVTFLVFFLMSFSQYDPAVAALGENSTPEALAAFRHEMGYDLPW

SEQ ID NO: 30 - protein, 239 aa, Bifidobacterium longum
MGVYGVNKDSVAARVAQAFPVTLQLTFIGLIIAIVFAIVFGVLAALYRDTWVDQIIRVVSIIAIATPSFWLGVLLIYVLQIKMAWLPGSGDLVPFTQDPGAYLARMAMPSFALGLPVAGQLTRIIRTSMVEELDKDYVRTAIGAGVPKSVVVSRNVFRNALITPVTTLGMKIGYLMGGAIVIEVIFALPGMGTAMFDGINGNQPMLVQGVVLVVALAFIIINIIVDLLYVLINPRIRTV

SEQ ID NO: 31 - protein, 674 aa, Bifidobacterium longum
MLFGKNKAAEAASRPGVKFNRFSKMTVGSKISFVVIALLIVCAVFAPVLSPHDPLEITMSYQAPTGEHWFGTDNLGRDVLSRVLYGARYSLVIGLSSIVFALIVGSLIGALAAVTRTWISELIMRVIDVFMSVPGIALAAVFVSILGQSMIGIIISIGVLYVPQIARIVRANIISEYGKDYVRAVIVSGARAPWILFKHVTRNIAAPVMVFTTLSVADAIVFEASLSFINAGIPEPTPTWGNILSSAKAGVIFGYWWQAMFPGLAIMITVLCLNILSEGITDAMVAAPTAPVTKSAADAEAERREDRLLTDPVAAYREQAESLADSLAALKEAELKRTDRFEPTSTAAPVIEVKNLCIKFPRHGDVNVVDHVSFAVRPGETMGLVGESGCGKSITSLAIMGLLDPKAEISGEILFGGRNLVGMSPKEHNALRGHEIAMVYQDALSSLNPSMLIKSQMKQLTSRGGTRSAEELLELVGLDPKRTLESYPHELSGGQRQRVLIAMALTRDPKLIIADEPTTALDVTVQKQVIDLLNELREKLGFAMIFVSHDLALVAKVAHSITVMYAGQVVEQGSTKEILTDPRHEYTRGLLGSVTSIEAGAKRLHQVPGTVPSPADFPKGDRFAPRSSHPDVGLNTRPIFERVPGTHHYYAALPADADVTPAAATVSTQEGGAR

SEQ ID NO: 32 - protein, 192 aa, Bifidobacterium longum
MTNETTAPSGTESRILDQIVAEITGVIAKMDEGDIERAMPLIGKTGRVYATGEGRSGFQARSFAMRMMHIGYTSYMMGETICPSMHEGDVLLAISGSGATRRTVEDAEAAKKLGVKVIAVTSKPESPLAAAADAVIVVPGRVKGEAGGSIQLLSSLFDQSVHIALDALCLMLSRRDNVSDADANANHANVGL

SEQ ID NO: 33 - protein, 282 aa, Bifidobacterium longum
MSHAAETPRKDPNVPIIELRDVEVVFTTRAGSGLFHKNRITAVNKVNLKLMPGQTIGIVGESGCGKSTTANVMCGLQQATSGKVLFKGQDVTHRTAKERMDIGRVVSVVFQDPATALNARMSVIDQLLDPLVVHKLGSKEERDRRAHELIRMVGLPTSVLGALPGQLSGGQRQRVAIARALSLKPDAIIADEPTSALDVSVRAQILNLLSDLKRTLGLSMVFISHDIQTVRYISDEVMVMNHGTVVERGKTMDVMRNPQDGYTRILMDAAPSLLHPTAAECA

SEQ ID NO: 34 - protein, 320 aa, Bifidobacterium longum
MATQFRGVIPPVVTPLTASGEVDKASFARSINRMIDAGVDGLFTLGSSGEVAFSTDARRREIIQTVIQVVDGRVPVFVGCIDTETNRVIEHAKEAKELGASAIVATCPFYALGGMAEVERHFRLIHAAVPDLPLFAYDIPVCVHTKLPGDLLVKLGRDGVLAGVKDSSNDDVAFRFLVDDNAKAGHPLTLLTGQEVVVDGAYMAGADGSVPGLANVEATGYVRMWKAAEAGDWATVRKEQDWLAALMRIVTVPQGVAGFGSGVGAFKTAMALLGVFDTNQMPDPVLPLKGENVKRIATVLEECGMKLERTPEEVSASTEA

SEQ ID NO: 35 - protein, 280 aa, Bifidobacterium longum
MNGTADRATAGSRATDTEPDAPQGQYSTQELAALARLSLGNWSTTQVSSRSRCDETMDAIKSYILRERLQPGDVLPTETQLCDTIGASRSSVREAVRKLEALNIVKVEHGKGTFVGSLSLDPMVETLAFRSMASVGKNFTDLQDVVELRRFLDLGCAEEVCASLAGTEQPRLTELAERMSAEAKEGKTFTGLDIEFHRGILDSLNNTVAKQMVRSLWLVHMAVLPQLGLAASSELDRTADAHHRMLNAALAGNVDDYREAVFDHYEPIESILKRRIVQEQ

SEQ ID NO: 36 - protein, 772 aa, Bifidobacterium longum
MAPWQTTDFPHGAGTVIAEFSVRADGPIWRAQVPATADGIPTAAPYLCLAVRDGHLTLTARSLSPDPADPNAQSHVSLDIEDAFGLDPGTIHEAALTFGAFGTRIYLDGYQCFACAGNLNPSRVHPSGAFDLGSPNAIACNAFLVDPVDWDAVRIAEHAAAAKPDIVFASDRLSPRDTDRIALADAGSVHARFRLRGRGQHGTILAAGVDGSERMTVAIGAGGLTLAMRDESGAGIACHAPGHWDDGGWHDLAIRSSRGAVDLFMDGVSVLHQPGQMWFADLAGPRHGRKGIDAFTVGRNIAGVRLMGEVSRGGLYVHALTDGQIARLAHTPPMVTTALFDAGYAGSASYRIPSLIRTVRGTLIAGADQRTAISNDAPNHINFVIRRSTDGGRNWMPMQTVIDMPGREDGLDGASAIDSCSVVDRSIGRITVLIDLNPGGIGLTNCERGIGVNRDGVLRLRDAQGNETTLDAVADAGDVWRSPMHADPSQRWHAVPTCYIAQIHSDDDGETWSPPFLIDAMVKEEWMHFMGVCPGTGIQLDKGPYAGRLLMPFYCSGQSRTHYSGGALISDDGGETWRCGRMINEGREINGTVVDPATMRDDDATTSETTFVQRADGDVVAFFRNQHASGRVGKAVSHDGGDTWDELEFDPALPEIFSQPNALAVPQLGTDAVLFANASQMMPYRGRGMVRLSEDGGRTWTGSLCVHPHHHVYQCMAICETTHDVECEGSQLGLLWEIETTGVYITHIPLAWFSHGHDKGVAANHTDDKESS

SEQ ID NO: 37 - protein, 167 aa, Bifidobacterium longum
MSSRAPRPARNLRYFHAIRHRHRLKQLPCQSRMAIKRLNTRHTFWPITPVTPPYTTKYGKRTEKLLTFVRSLVATPAGLEPVTSAVTGRRSNQLSYGAICTSMKHKRIYYSITGPRRAARRVAPRQCQAHPLPLQAIATPAIPALPIPWWAVVWRGFDRYADRAAQA

SEQ ID NO: 38 - protein, 190 aa, Bifidobacterium longum
MKPRRRRIAAMVTSLAAALVMSLSCVTTASASSVYLSVPIYVQEQSNWCWAATSKSVSVYLGGSNSSQCQYVKWGKNSSSCANVTGDLSTDVRRALSSAGIRNTGSMINSAASTAIVSGQINNSKPLMVRWGWDSGGGHMLVIRGYTSDPGYLVVSYIDPLQSYYSSGTYDWMKSGSGHTWTHTRYGFSR

SEQ ID NO: 39 - protein, 262 aa, Bifidobacterium longum
MSTFYIVRKSLIQALRILFAAMLFVVAIGTVCPDIARAENLPESTQINEFAQSDEARTQTLALMEAVQNDGSPEASTQIAIGYADKVHYLRYDDSGAITNTITDPQDYDWVAPVEVEGRLVGRITIWDNNGSLEVGNFSPDIEEASLLDDDDSTTLISDGFSRAYYSMENSRISPLNEAARAIVPESVQVEQGDIAIRKNAPSGFDDSAGGGSIGVVSSDTATGTDSSMNIFMLMAVFIVVTAVLALMCRILWRGPNRRNDR

SEQ ID NO: 40 - protein, 131 aa, Bifidobacterium longum
MRKRQPLFSGLCLAVFALMSGCTGAPVQSDDAGAHLEFRAYAESGTVRIESQSETVLETRIKGDWLKVAPDDWLENDRPLTFTVERDSGGVEDNVSCEIIFSGHTIDERRVTGPETSATCQYDTWRDAIDY

SEQ ID NO: 41 - protein, 373 aa, Bifidobacterium longum
MADSPASDIKPKAYRPRIVDEWQLAPAIWDEVRHRVDDDSANKGQWILTGSSTPLNENRPNHSGAGRIGRIRMNPLTLYESGLSTGAVSLSGLFEHKFAAAKSEISTQMLLDAVCRGGWPEAVALPVSDAQILIREYMRLTLTESVPRQGKDPDVARRLLDSLARNISQAVTFKTLRKDMYGTEENLDDFISERTVSGYTAMFENMFVIDPIKGWVPPARDPKRLQTKARRYFADPSIAAAMLGMSPAALIGDWQTFGFLFENLCIRDLLVYARSLLDIGIEPVRYYRDDSGLECDAIIELSDGRWGGIEIKSSEDKVPEASANLCRLKDKLLRNPSARTREPEFLAVLVGVGEFAYQRDDGVYVIPVGVLGA

SEQ ID NO: 42 - protein, 354 aa, Bifidobacterium longum
MKGSMMSVRYGAKRVIASSMVFSVLCALAACGGGSANSQAGSVSVACSQQEDFCQAMTAAFQKETGIKTTYVRLGAGEVLARLETASGEFDVWAGGQAENHLLADDKGWVEKYVSPNASDLPDEYNDDNGIWSGFYTDSIAFCSAASELEKKGLEAPTSWEDLLDPALKGSVAMPHPATAGVGYMAMYALAALNNGDEDAAISYFKQLNANVMQYSKSAATGTEQAGRGEVAVAIALDSDCQKAIAAGYSDLKTTYPKEGTGYEVGAISVLKDAKNADNAKKFMDWILTADAQNLYADVPSYAAPTNPKATVGADVPRQDIVKKVAWDTRKAADGREAFIAAFESDVASADSAQ

SEQ ID NO: 43 - protein, 396 aa, Bifidobacterium longum
MISPTYEFPENFIWGAATAPHQIEGNNTASDWWAREHSPRTDVSEPSGDAADSYNRYREDIRLLADSGLTMYRFGIEWARIEPVEGRFSKAELLHYRAMIDACREFGVEPMVTIYHFTMPLWFAAEGGWKRPDALEKFERYVRYVLPILNDVTWICTINEPNMVALTQGGTEGTDFVAASLPAPDPVISKTLVDAHHMSRAVIKSELPDAKVGWTIACQAFHAVPGCEKEMEEYQYPREDYFTEAGAGDDFIGVQAYLRTFIGKDGPVPVDDDVERTLTGWEYFPPALGIAVRHTWDVAKHTPIFVTENGIATADDRRRIDYTFDALAGLHDAMDGGIDVRGYTHWSLLDNYEWGSFKPTFGLIGWDKDTFERHPKASLNWLGSISRTGVVTHPYR

SEQ ID NO: 44 - protein, 291 aa, Bifidobacterium longum
MQSAGRPAPAQDNKLPLWQRFIKGRGIAYIVLAVIGVVWIFPFLWMVLGSLKTQREILAKPPKLMPEHATLANFSQWFTQLNFGSYFTNSLIVAVITVLGNMVFCSMVGYALAKMKFVGKNILFGAVMVTLMVPSVATFVPLFVIISNVHLANTYAALILPFLCQPIGVFLMRQFIGGIPDALMEAARVDGAGELRIFFQIILPQCGPALATLSILTFLSSWNNFLWPLVSAQSEEMYTLPVALSLYSTGQNATNYSVLLAGAVLVITPILLLFVFLQRYFIQGVAMTGIK

SEQ ID NO: 45 - protein, 305 aa, Bifidobacterium longum
MAKRTKAQAKGAGSSLCRRQTLLAWGFALPFAVIFCVFMLIPLISSMAMSFTDITSRDLRTPFNVNFVGLDQYIALFGDKRFLHSLGVTGIFVLIGLPITMIIALAFAVALNKGSQHLNAFFRALFYAPVVASVVAVSVVWRYILQADGLLNSLLSLVGVQGPDWLHDTRYALPALMIMTIWRNMGTLMIIFLAGLQAIPEELKEAAAIDGASKWRTFRSITLPLMKPTLLLGAVLLSVGYLQFFEESFVMTQGGPLDSTLSAAYYVYQKFGFGQYGIASAASWVLFIIIALVSVLQFRILRSED

SEQ ID NO: 46 - protein, 431 aa, Bifidobacterium longum
MKRSMFKMATAIIASAAMLTSVAACGRTSATSDSADDVTTIDSGKATGDLTIWAMGNEGDLLGDFVKDFEKENPDVTVKVTAIPWSSARDKIQTAIAAGNGPDVAQMGNTWMADFSNSFSTVPSNFDMSGFFEGPADNYKVGDQQLGVPWYVDTRVLYYRTDIAEKAGITEAPKTWNELKTMAEAMQKVDGVDYGMRIGASGTDCFIGFLPYAYSAGAALSDDGQTKWTIDSDAMAEALDYVTGFYKDGIADTNADVSAGADIADFVAGTTPMMLQGPTAVSQVEELGGDDIKGKYATVTLPAMDDSSDMGTSYLGGSGLVTFKDSKNKQAAWKFIQWTSQPDVQAKWYTLSSDLPAAQSAWDDDALTSSTTLTAFGDQFEHAQGVPAFTTWAQVSSAADRTFEQIAKGQVSVADGLKSLQSEADSIGIGE

SEQ ID NO: 47 - protein, 375 aa, Bifidobacterium longum
MRRATVYDVAKKAGVSTATVSFTFRRPDKVKPSTRAKVLRAAKDLDYVPSANARGLARGNTGVLGLYSFDMLIERPLGDEDDFDSYSGGNDVVSENGKPIFSNREGGSFDCPSVLSYPLYVDEVQRGFELECRRRNRAVLLGTAIRHDDGTGITDVAGRVDGLAVFPNESTNTMPLEALCRSIPIVRLSEGDGDEPAAYISCDNETGMNQLIDHLVDVHGVHDMEFVGSLDNYDSRHRFAAMRAKLKMKGLRVPDLPLDDSMSGTHEWFVDLCEVIDAGRLPQALLCATDQTAFEVMSILRDADVRVPQDVILTGFDGVLAGQVLTPTLTTVRQPLELLGRLAARLLDEQAGEPWGKPERFRLPVKLIVRGSCGC

SEQ ID NO: 48 - protein, 70 aa, Bifidobacterium longum
MEPYSAIQVTADMTNAHTQKREITALEQTMHRMSDVTGTIITLREEGTIPTDAGDINVIPAWKWALQSKN

SEQ ID NO: 49 - protein, 82 aa, Bifidobacterium longum
MTSLSKPAMFNLEHILTGMWRSGKTFQLFQLINDLMQSGVPRERMFYFNFSDERLQPMPEDMLDQVITEFWRQDPSSRTQGA

SEQ ID NO: 50 - protein, 388 aa, Bifidobacterium longum
MIPRTIAQELTPMLSWFPVVSVTGPRQSGKSTLIKNMLPDYEYVNLEDETTRLSAIEDPVGFIHAHERKLIIDEAQYAPSLFSQIQVMADERGTMGQYMLSGSQNFLMEKRIGQSLAGRVGMLQLLPLSYEEALQAKTDLTVDEFMFHGGYPHLYDVPTPTDIYFRNYTATYVSRDVAEYLDVRNLTDFNTFLRLCAENAGNLLNLTALARDANVSFNTAKEWLSILEASFIVFRLVPYSANTRKRLIKTPKLYFYDNGLLNYLLGIHSPQELADDPKRGDIFENLIISETVKRYRNRNKDCELCFYRDTNQREIDLIDTTQRRNPTLIEIKSGMTARPDFFKHLATIGEELGVPTDHRIVVYRGTESFTSKNGRTITAKDYLCLAHS

SEQ ID NO: 51 - protein, 567 aa, Bifidobacterium longum
MTVISSRSKIMGGNGRRGPRKSPSVWVTLVGVIAVLMLVLGLPVYKLFVAALSEEGRSAMVGAFSNGGETLINSIVLGLLVGVLGTFIGFVCAYAETFIQFPGRKALHWLTLLPTISPPFAASTAIITLFGKRGMITNGLFGLEVNIYGLSGLVMVLTMTFAPVAYLNIKGMFENIDPSLFEAASSLGASQLRTLIRVTIPMVMPAMLSSFLVLFVEGIADLANPLVIGGDYRVLASQIYFAVAGSGNIAGAAGVAIVLLVPALSVFLVQKYWASKKSVVTVTGKPTGSLKPVTSKAVVVPIMTVVTLWTLFVVSIYVTLFIGGFVKILGVDNTFTWEHFRFVRRLGSDAIITTLTMTLIAAPLAALLALAIGWLVVRHLPRFGKILDLWGMLGVAIPGTVLGLGFALAYSQPTVLFGVNILPALAGGLAVGNGAIAIIMVFVARGNPTGQQAFISAFKQINPQVEEAATSLGANPLTVVRKVTLPLMSSAVVTAITYGITKSMTTITAIIFITTPQTKVMTSQILDEVDAGRFGNAFAYSSLLIVLVLIVLGIANIFLTRLNQSKR

SEQ ID NO: 52 - protein, 36 aa, Bifidobacterium longum
MRYAFVLTHLSPYIFRTVHSRAYTHLYTDIYGTNFN

SEQ ID NO: 53 - protein, 111 aa, Bifidobacterium longum
MHDEVELTRGYLLHRLSHEDERIRTLLTAHTTNKHYIEGPQIFTLSILNMLGVSGKTNERRIDYFRCRNMVYIFHPAFELRRNHMMKLNPFVVSQRLAREATTEHIFHNVQ

SEQ ID NO: 54 - protein, 63 aa, Bifidobacterium longum
MTISISDNDRQESSFFGKLDGLHSPTFSATSLKPMHGNNDSLRRVGNTIPRVNLYPRVMVLII

SEQ ID NO: 55 - protein, 98 aa, Bifidobacterium longum
MFPDIFIRSIIWRKQDIPYIAVNKLLIPSKIVRRKHGLRIHRGSRIELAEKLGTIRTTTIERRSVRKTRILPLLTHEYLIVQGTPRLNQRMIVNRSDD

SEQ ID NO: 56 - protein, 379 aa, Bifidobacterium longum
MAKRSVKQRNLNIEVLRILAMFLIVACHATLHLPWLLHVDSNLDFLPGWKSALAYLVVQYGQVGVSIFFIISGYFLVRKTFTWQRIFKTWFQMFCYSFISLIAVLIIARFTTLPNSIAPLLSGDDLWRTVLWSIVPFIYGSYWFITAYVCLLLLAPFINCLFKHLSRRNMAALIIVLSFFSIWILLGGRTTPWNNVVYAVLGYIMGGWIRLYWYEVNDKIKSSYLWGIIVLSTIVMTVFNHYAANRTWLATFLGWHEQIKPGIQIFPMIIGSSIFILFTKLDMTSIKGFGHKVVLKTASATFGVYLIHENMFWYRLIWPTIAAIFPTPNSFISTVSVAFAIVLAVFISLSLIGFIADTIIVHPLTKIILKGWDTPHHTN

SEQ ID NO: 57 - protein, 567 aa, Bifidobacterium longum
MEGKPVEQETLVHTQKSLNKPLIVISLVTGFLIGFGNAAAATAEFPVGSVLMWAQCVIFSVLVYIFFSLTERLLSYCRHNDCQKWQKRMGRIDFSFSPVSLTIVASFIFLMWLPYLVILRPGVIEWDAGDQIAQGLGYSAFGQEPGQIYDHHPFLEAIIFAQFIKVSIAITGSYKLGAFVLVTLQCIGMAVAFSCLIAYIRENLKASFSIALSSTLFVALFPVFPFYFSTVLKDSFHALFLLPWAIMYVEMVRTRLNCVAKASFSVTFIIFSVLVCLTRKTGPALVFLALIMLVFVKTSVWKKLAAVVTAFVIFLSMSSFLPRYVYPALNVVPTDSEQYYIIPLQMTARWGKDHPGEATEKEKNIVSQFNIFTYDEMTKNYEPFLTDKASMYKLGDASLRNDYFKVWLSQGLKHPKSYIDAFAALESGWFAISKSPTGQPVYPYDTVGNQMTVFYKTVTNPDTTSEFINSPNDSMNDSMGRWFNYFKQIPVINITTYTAFWTWLLPMFAVYMMIRRNRVSLLLLQAVPFLLGIASLYASSTAYISRYMLFAMYLAPLLIGIISSDQE

SEQ ID NO: 58 - protein, 161 aa, Bifidobacterium longum
MRRRPGSANPMACSGPGNATADGSSKISAVRFRAESSCGRFDDTAAVPSGLSASPDYRSNPHGGTMTRRTSMRKAPADPLPENANRRHAPLKRRRRWFRMVGGPDRPILLDPPPGDRRTPVAAHDGCRPMPIQGVGRILQKTGSIVRPNMRISVRRRGYLA

SEQ ID NO: 59 - protein, 842 aa, Bifidobacterium longum
MMDFEPIAIIGRGCILPPHSCSPEELWDAIVEGRSGIHPPMPERWGTRVDYVDPDRNAVDRTYCGIGGFVTDYDQDERAASSERMRLNRTQRMIVDSASQAIGEAGIDRSERARCRLFVGNMLADEAFGDQSLSEISGDVLDECVNEFGADARDAARRAIDDVILSRNDASETANAPSDLARIPAEVLGMPDDPIVIDGACASGLLIVDLAARYLHTRAKPLAMAVGAMANMSITGNVSFAKIGGLSDKPARPLDANANGLVPAEGAAAVLLCTLSYARLHGYPISGVIIGSYTTSDGHGKAIYAPNPEGQRCAMRGALAQAGIDPDGIDYIETHATGTPAGDNSELTAIIGMLSERRNGPVSIGSIKNLIGHGFPTAGTSNLLSVLESFRHERYLPTHGVTTPHPLIAKHPELLQLHDAPDPWPEPGNRPRRALINAFGFGGINSSVIVEQYDDGRPDGPAHADAEPRDATYLAVQCVAQANCEVPRTLLEDPMTADWRVFHTPPVLLPHMDIAQRLAVLAAGNLQPMMAEPDRKETIGAFLGQPSGLAVGARRELRIRLPEILDAITHAEIPDERRRELRRWFADRVTTSIGATVEAALPGYMDNIVSGRIANMFDYRGPNCVIDGGRFSFARSIEMASLVLAEHEADGTIVGSSFANPSHLVDQEAARLATATLIMLRPLDWAEAHREQVRAVIRISHADRPGTEPQTLLDAARLAQAVTGHASRMPRLQAGDMLVEVMDPDAMPSERIGHRPDGLRADPGTDAGHGPSDGVTVHDTWIGVRGATIPDCLDTLLDGTEPVQPDGQATPIRIIIPFDSPAEKENTMRELRILKDALRHHR

SEQ ID NO: 60 - protein, 209 aa, Bifidobacterium longum
MMRRARLFGPLLASGCEVLISGEGTRRVRLFDGERALVGNATAKRRREFTETRLLAHEALRRIGHDGPILKGSDGEPLWPSGIVGSLSHCPSLCVAAVASAERIRAVGVDVDDSDGLSDGIMRFVFSSEELASLRMASSVERRAAFCAKEAASKALSALDGTGDFRKVSVSLKADGTFAAVRRDVTFRGQWRFYDRLVSAMVAVPSETV

SEQ ID NO: 61 - protein, 391 aa, Bifidobacterium longum
MTIIESQSMPETVEKDRRLAKSGSLELCAISKTYHGKNGDFCAVQHTDLNIAPGTFVTLLGPSGCGKTTTLRMIAGFEQPTGGDILLDGESILDMPADKRPMSMVFQSYALFPHLSVRGNVEFGLKLKKMDKALRCKKVDEALEMMGIEQYADRYPHQLSGGQQQRVALARALVMEPKIILFDEPLSNLDARLRVKMRGEIRALQRRLGITAIFVTHDQSEALTMADVIVVMSAGRVEQIGSPWDIYHHPVNRFVASFLGTSNFLEGQIQSVENSGASDNAMAMYSVSTAFGDMAVAGVTGMQTGDKVNVVVRAEDLLVGGEAGAGRMAINCSVVSSAFDGQVVNYTLDTATGQMIGSAPGSMAPAGSGAEVTAVFDCRALWCVPVEKHGA

SEQ ID NO: 62 - protein, 289 aa, Bifidobacterium longum
MFTDGESHKQLRRLVGTIINTRYHAINYTWPQINKNCDFTTEYARPYVCGILAQLVGVSVEDISRMVSASETINSFLLRERLTLDDIEQVAHSIEYAYQVVKEIEDKHVGEPLYIGNELLDLPQETRYPLIINLVTDGFAPFVAALDFLAFNLLTHPYLEKELNARAEQISLESLRLFPPFTTISRTCVHEIPFKEKIIRPGQLVILDLYSINRDPEVFPDPEKFNLENTARAYSFGAAQHLCSGNPLVRKALEQVTRQSESLYKYKIQSSCFKNSYGFTDMNLSIELK

SEQ ID NO: 63 - protein, 302 aa, Bifidobacterium longum
MNDGISDPQLKGKIPVKLLHDSPYLDHKLDLYLQSNPNARPRDILVYLQSIESDARALLKSIRIPQNLTSSNSMETIIGREKEIQLLDIYLNRRYKNNVILIGEPGVGKTSLILYYFKSHHLPLMSVSAAEMLSGTKYRGEFEKRMQHVLETAQKEGSAIFFDEIHTLIHAGASEGGVSAANLLKPIITRGDIKVVGATTPEEAKTLYADGAFERRFSFLKLKEPDMQTLRLIALNFVKESGDNQCFPSSLFEEIVAFLDEKFPNRHYPDKLIDFIDFYLAANKQANFTLHEATVLFAESQI

SEQ ID NO: 64 - protein, 325 aa, Bifidobacterium longum
MFEEKDSDVILTILNIFKNPATVDDVSTALLDTPYEVDDIASLVQRLFENNVIKEYKDITALDTTLTPKQLAKYDRQLRNYAVLPNFTINDAIKQQERLDAASILILGAGGIGSYLAIGLAQIGVGTLHLIDFDEIELSNTSRQVLYREKDVGKSKIEVAVKNLTEVAPDATVVGHNLEVTSVNSLTEELKSDQFDLIAVCADKPLGKLVYIVDEFSKLSGTPVLYGGPYADSKIFLGPLIIPGKTKSYSELVPSSYADTSNPKVASINEYRETAIVDTDNALAAKMMEVEIIKYIGKLMDPSVVERQIVLDTWNWSFHDGQFSK

SEQ ID NO: 65 - protein, 143 aa, Bifidobacterium longum
MPGSTFQHHATRAENVWRVTPSRSATSPMDSLPVTHARNARNSAMRLESSRLAVELDRQPSHRHRCEPASVKPFLRIARPHNGHLLALEDLFLLMEQVNTKSQAPGRINDGNVNHTPTRKTGVSSTLDLRHTSLYLTQNMSHI

SEQ ID NO: 66 - protein, 399 aa, Bifidobacterium longum
MIKQDALTRRASIAYYVSTAFLIASKSFPHAVLTVLLLHKGLDLTEIMFVQTAFTIAVFLFEFPSGVISDLYSRKIVYLASILAWVAACSVIVFGTGFAMMCVAWALYGIGEALASGTVDASLINLYKRLSPDPDEEIKTFKRISNQISMVSMIIGAMLGSALYFTIGYNIYAVAMVLACAAALPIVLAFPKDEHDAREKKPTIMSQVRDGLSELRKDRRLTFLIGMAAVSQIFFQTHFNLWQAYLLLMGVKDKYLFVFYLVFQVIGIAAYAIHIDGRLRRLLYLGIPVALIMPLLITSGSRIVSIGAYCISVFIFMFLQYMCDVLFSIRVSEERISTLITLNSTTCRIIGFLVLGLNGLLLKQIKLTTLIVGSFEIATFLSILLGLLFMLSFSKKKKE

SEQ ID NO: 67 - protein, 206 aa, Bifidobacterium longum
MKRFFDRPDTHWRNLSGALHVYALPDANSYLVKEARETASWLNGAVELAVQPVDYLHMTIQRLDLYREEIPAEIWDELTNHLAMSVADIESFDVEYAPATVRAGAIEAVSDENPSWKRLVDAVRESFCNVGLKHALVDPPFGPHYTVAYCVQDTDAQRDDELCQLLVDAPATSMRVSSVDLVAVDQSPEEGVFRFSSLMHWPLRGA

SEQ ID NO: 68 - protein, 46 aa, Bifidobacterium longum
MEVRAGRPEYLGGRLLRYLPPFVACLPIQPTGAASSQRMYSLRLMW

SEQ ID NO: 69 - protein, 341 aa, Bifidobacterium longum
MQRKHRVSMADIAKETGVSAATVSRALNNHPKVSAEVRAAVLQTADRLGYIRNLGAASLAASRSMTVGLLLRDMSSQFYGGVAAQVQMETDAAGYDLLITIGGDDAESQMNAIRNLLGHDVGGIIVASGRIAEEVMEYSARFVPTVALSSGLDMPSVGSVRIDPKCEADLARRVVVAGHRNVAVTASSNPLASTLHARTATFLTELIVAGAQTLIMSMPVEQGRYLREQVDRALEAKVTAIMAGSDAIAVSIMEYLQELGLSCPDDISVTGFDGVGALRSPLLGLTTVEQPMERLASAAVGMITQYLTSGGSEEPISGTVINQLVMGRFVPGRTLGSPKAV

SEQ ID NO: 70 - protein, 236 aa, Bifidobacterium longum
MHSYKYSLLGVVAAVAMSLTAIAPASAEPIESSNLSSNSITLSSEDKMEISDILTSYGVDEEKAQYLVSRYEHGYAWDSFTPGKQPIAATQRKTLYSVETVKTYEDGSIAVSTVPNFEALADAPQTRGITGCQYHQSGSTRYWKNCDGTVNLAVISMGFNFNYQNVNHSNPKITRYGPYHHHIIGGALSNFRFDRISDSQVRLSADLDVAFRGFPAGWTAWMQVNVTGDNAWTSNN

SEQ ID NO: 71 - protein, 73 aa, Bifidobacterium longum
MWDYARMAQIAGKLGGPARTGALIFGAGMILGGVIVDGAHQAMRQVDEESRRKADMAAQARRLENQVSGRDDN

SEQ ID NO: 72 - protein, 44 aa, Bifidobacterium longum
MHANGLRSFWAEAIVVESGKRRDYCSRNTCGAFMKFVAQCSPQK

SEQ ID NO: 73 - protein, 94 aa, Bifidobacterium longum
MSSTRTIETVKASDIDEAFERGDDVRRYFDMTKPRVIRPAKTKTRKVNLTLPDWMVESLDAEADELAVSRNAVVNTWLAEKIAERRKEQRLLTV

SEQ ID NO: 74 - protein, 271 aa, Bifidobacterium longum
MEYAIQLSLGVSNMYAIFTVRCNTCLMTSETLGNMAASSETSLHDRLLDEWGMAVVSGTVSAGERLPEPDMDGNATPSRTVTREVTRVLESMGLVTVKRKAGATANPIEAWNILDPQVIQWRLRGPHRIDALHELSQLRAAVEPMSARLSAANATPEHWATLTRAAIEMVAHSDHANESEYLDADILFHRTLLEASGNLMFAALGDVIASTLTGRTQHELMPQVADQTALGWHTEVAALIRKGDGDGAETAMRQIVDESDQAISHIAGTEA

SEQ ID NO: 75 - protein, 474 aa, Bifidobacterium longum
MNTLVLAEAAQAVQLNSTQLGISVVASIVVLILLVTVAKLHPFVSLLISALVVGIGSGYGPVATVESFSTSFGSTMASVGILVGLGAMLGRVLMDTGAADSIVDTLLAKASPKMIPWTMALIGALIGLPMFFEVGLVVLVPVIILITRRSKLPLMRVAIPTLAGLSVMHACMPPQPGPLAALSCFKNGSVGVTMMFGLPIAVITAALVGPLFSKFAAKWVPVGAPENFDTGKGRVDADGNPITTKPPFSLSVLCILVPAILMLGNAIFEIVAPDQAGSDAVYAQILAFFGKPAIALGTAVIFAMIVLGRTTHMSWKTVNDSLKAALPPIAGILLIVGAGGGYKGVLVDTGIGDIIGKFVESSSIPIFLLAWLIAAFVRVATGSATVAIITTAGILGPVVEQMGVTTPAIALLVIAIGAGSVFLSHVNDAGFWLIKEYFGLEVGETFKTWTVLECLLSVVVLALVMICSIFVPLV

SEQ ID NO: 76 - protein, 297 aa, Bifidobacterium longum
MQMGMIGLGRMGGNMVKRLRDGGHDIIGFDMNPDSGRDVASLEDLVAALAAPRVVWVMVPAGEPTDSTIARLGELLEPGDIVVDGGNSKYTEDREHAAALAEHGIGFLDCGVSGGVWGAARGYALMIGGSDKDYAAVLPIFETLKPEGEYGLVHSGPVGGGHFAKMVHNGIEYGMMQAFGEGFATMMRSEYVTNPAETMDSWREGSVVASWLLDLFDNATKDDPELKNVPAVANESGEAKWMIEAALELGVPVPTTAAALWQRQTSRGGGDDILRVVTALRAQFGGHVTKVDEIATH

SEQ ID NO: 77 - protein, 447 aa, Bifidobacterium longum
MKLQRKALKTLKQWKTTPDHKPLLIRGARQTGKTWLVNEFANGQYDNIVSVDFMQRPSLSGIFEQDLDPQRIIRQLELAANQRILPGRTLLFFDEIQESPLALTSLKYFTEQAPDYDIIATGSYMGISKHGKTSFPVGKVTMMNLHPLSFVEYLDSIGQDMIADTIREGRFEDIPQALEPQMNDLLKTYMWVGGMPAALSAHLDNGIPQDVRAVQQDILNAYDLDFSKHAAYTLGERIRLVWNTLPSQLAKENRKFVYGVVRQGARAREYEEALTWLTDYGIITKVPCLDALHIPLTGYESLNTFKIYLEDTGILGALSGLDVNTLVNKSKLFSEFKGAFVEQYVCQQLVAQGIKPRYWANPNPQGNAEIDFVMEQGDEVFPIEVKSSSNIRARSLSYVCNRYGLHGIRIGEIGYRKQSWLTNIPLWCVDGLGEYLKRQIEKSRAEA

SEQ ID NO: 78 - protein, 522 aa, Bifidobacterium longum
MKLRKLFAGVAAAATLFGGMAFGATTANAAATDAATITVNNAQVGYTYTGYKFATFDNVQGEAPNATSVEVNTVAAWKDAVYQAADAANGNAAVPAEYAENPAAYVATFDAATARKFTDELTKHIPTGEQGTAAVNGVITATEGWFLVTSKAETAGKSALVATQITSGGETYTKITLNTEDGQHNIDALGRFNAKDENVPTPPTKTADGQGTVNVGDTVNYTITAVVPPAAAGYDTYKYTITDAASKGLNVAKGDADFVVVVKGGNADGTDKTLAESTDYTLTQAGSASAVNGTVTTIAFPNVKDYAGKTIQVTYKGVVTSDAVDQVTNTATVKNNNDQTGEGTPVVKKLGKFDFTKIGVGSDAEGLAGAEFKVSADGGETFIKFSQDANGVYYPDANGNETLTSADGQGAQKTLGKVAVRGLAEGTYTVQETKAPTGYAENFKVTFTVTIGEDGGEGTLSADVLQQVNTTNKTVLNVKSITQLPLTGAAGTALFTVVAVLLAGVAATVFAKSRSTKRALNA

SEQ ID NO: 79 - protein, 422 aa, Bifidobacterium longum
MRDFELVKLSEDEFDKFSACHPQGNFQQTSAMGTLRKGEGKTVDYLGVKEHGELKAAGLLQIIHAGGSTFALIHDGPMCDFDDKELLAFFVGKLKEYAKQGGAAQLDITPEAVYQLHTQKGELEGSADDEMVANLLALGFDHVGGFSTGYTSVPRWRWVKDLTGIKDEAALTASYAKYRRRNVRIARESGVHTRRLERDELSLFHQLCELSCEKQGFENRPLSFFEEMYDAFGDNIEYRVAEIHFDEYLKTWQDKLDKLNADKARIQKDLERSRTDKRTNQLNLQLASVDKNMPPVVKRVQEAHDLLDQYGAVVPLDGSMFLYHPREVVCTTSGADERFDKFYAPALMHHEMMVKCIERGIPRYNLYGINGLFTPENNPGFGVLEFKQRFNGFVEEMPGEFVLPVKPLVYAAKQLAHKLLHR

SEQ ID NO: 80 - protein, 399 aa, Bifidobacterium longum
MSIPSQHVKARLQNADFFEKCTFAFFLITFVLKALTDGFFITQGVKGAITDSKYLTMGAAIFFGIVYMVQRRRNRVFWNEFRQLITVALCFVMATLVLVIAQNHFVQWQIRDILNLITPMIFAYVMLNVLSFEQLFHGMKIALVFSITGYITQLVLRGVTFGDIFASSFSDSTSPLESNDFSAIAIMFCFFFCYYRSSRWLTVLSTLYAIATFKRMAIIFAVIAFFFPMLFNRDAELPKWFSAISKIVFFGIAMFYCYLMLPTSTALQSALHLDIGEMTMGRSDFLASLINQGYQSFGFGSVEGTIGHSLEMAFVRMTFELSPIAVLLFINNYWNITGRNLYCSLLMVFNFLNLTTADSISAMFAWAVCYILIGMVVYAHGPAVTAAKQSRLFAKWQRG

SEQ ID NO: 81 - protein, 273 aa, Bifidobacterium longum
MTMHSDNPRKAYITICTDDKYLPGVVALNRSLRTVESEYPLIVLTTGNMSESGVQTLANESIRHLTAKNIVPSEYIRNLNIKNGSPNWSNTFFKLRIFGLSQFDTLVYLDSDMIVLRNIDHLFDKLHLSAVAAGHHFNKTWNQLNSGLMVFTPSIALEKNLVDLIEGEPSADMLNGQGIGDQDIINHYFDDWDRQDNLHLPETYNQFISLVPEYLRKGYLATTKDIYVVHFVGKVKPWNYTIKEYLHMLLRALRWRSLAEFSIVRTFNRLLRK

SEQ ID NO: 82 - protein, 373 aa, Bifidobacterium longum
MVSLQKVLPRVLPCIPGNDLLYRILRINAVNAEPLACDVDALDELSDIHPRSSSPFVPDYRVGGASDYDLSLIVPCYNVEDYIDECLTSIFGQETHYSMEVIAVDDGSTDSTADKLNQWKQRHDNLVVYRQKNAGLAAARNTGLDHARGSNIMFLDSDDMLAPNAVELLMDTLTSSSADYVSGSYVRVNESGKPISKPYQIGSCGMPWGRVYRASVWDSLRFLEGYWFEDTLQAYCIVPFHRETRQPLAQTRYRIRGNSISHDSARRNKSADAYWVVEATLEQCRMLGLPIGQTLYEQTIGQFGALGASRIDGWSEANRRIFFLACSNLITTTTEFTGLTTKRALVWRDMELALRTRNYRLWKLACYFAYIGR

SEQ ID NO: 83 - protein, 352 aa, Bifidobacterium longum
MTAPQTDIRRHYNDLDGFRAIAAFAVVVMHVFLRGAYGADLAHGDGTDLLSLIQTIVSSLGTFVTLFFIISGFGLCCGYYDRIKNAEITPERFYTKRIAKLLPFFALLVLLDIIGTGGKDSLWEAFADITMVFNLLPDLNIEVIGVGWALGVIFLFYFLFPFFVYTISTKRRAWLTFAVSIALSLSCVFYFHKTNGNLDDRRFIYQAMFFVAGGLLFLYKDRIGSMGKIGRIVTLVIAIGALPLLYVSGPAWTTNLRQLVLWVPWMVFAIASDHRIFSNRIAKFFSGISFEIYLSHLFIFQVFNMLHLTHLTGIPSVDYLMTLALVIVGVTGFSVLAKRAIDYGWSLWRKRR

SEQ ID NO: 84 - protein, 369 aa, Bifidobacterium longum
MKIVFVNPIVYTPENASIPKVDNITSTMSYDLCLAFQRAGIDMTLVAAEEWKPIRQTDFPFHVVWMKSHWKRFFPIHRIPVNLGLIRYLKHSDADLVITSEVFSVDSLICSVFARHKTIIWHEMAKHNRMGGGMLSRFWYNVIPKLFMRKVLVAGRSEEARAFISRYCARVSETVIGHGVNLDVFRTAFHKTNTFCVVSQLIDRKRIDGIIKAFDAYVRRYDADCKLYVIGDGDRRTDLERLTQSLGLENSIEFMGQLEHDELQRYLSEAKAMLVNTSKDNSMLSIVESIASATPVITTSVPLNAAEIRSHELGIVKDGWNEDDLAYLDAHLDELVEHCKVYRETLSTDYKVRQFMKLYDGCIRPQRKG

SEQ ID NO: 85 - protein, 357 aa, Bifidobacterium longum
MMHTISDSYGEWNGDQMTESIRPLVSVVVPVYNTKPDDLRSCFASLSQAKDARLEIIAVDDGSRAETAHLLDDIAAECSNTVHVIHKTNGGQSSARNRGIAEARGEYIEFVDSDDYVDWDAQQRVLETLTSHKPDILQINVVGMTEAGVYFWPPKHGDGEYREIDKREIMTECAAMWAQLVKRELFETSGIVLCEGIHIGEDFASILSLATVARSAAVLDVDLYYFIDHDSSITHIPHPQMLLDITHAMDFVLEHVGDDLEKYHNEIEYQAIKQVRYAGVVRALDWEGIHSKVIPQLIEYMETHFPSWQRNPYYCQEAANQLKYRLLIGGHYRLYILLHQGLHALRNSGGIRSVLLN

SEQ ID NO: 86 - protein, 380 aa, Bifidobacterium longum
MADKSVFVDCFLSHNLGDDLFFFTLVSRYPKVNFTVYADRSYEYLSNRFPNVKLITSVESSSSRFGTADKIMRVCSAMRQRVALIREADAMVTIGGSIYMESKARGPKERLQRLYRSCKDASYAKAAGHYFILGANFGPYYSQQYLDSYRRFFERRCDDVCFRETYSAGLFPSVKSVRSAPDVLFTADLPSVPKRRQAFFSVVDLDNDGKFGALRDRRRQYEDWLLRSINECSQAGYDVVLASFSEPEGDVKAVSRLAEEASRQGSDVQPLFYTDNMDEVLRELAASEIVVGTRFHATILGLVAGARVLPIMYSDKTKHVLEDIHFDMTDAVDLKRATDDELVAMSPVRDATSFDVHDVIAAAQGQFAALDTYLSTITTS

SEQ ID NO: 87 - protein, 498 aa, Bifidobacterium longum
MFANALAFTVQFGINFFLTPYIVSTLGSEAYGFIPLVNNIIGYASIITVALDSISARFITIEITRGNYQKANSYFNSVLLADTILALLLMMPSLLFILKINDIINVPVDLLVDVQLTFFFAFLTFFVNLIFTVIGCCYYVKNRVDLNAKRSIESNIIRACILIALFTLNKPHIFFVTITTAVVAMYLFACNVHYSRKLTPELRVDLHKFSFLVIKEMLSTGVWNSINALSSTLLTGLDLLLANIFLGASQSGEYALVKTVPNFICQLVIVVLSAFVPEFNILYAKGDKKELLKSVDFSLRIMGYLVTIPIGFLIVFGKEFFSAWVPGQNVDLLQQLSIMTLLPLIAICGTDSITKIYTVTNKLRTPAIFMIIMGILNATGDYLLFTFTSLGIWVIPCVSFIVNIIIQLLFTPIFGAYCLHLKWNTFYLSIARSCSCAIVVISVSLLFKFIIQPQGWFSLFLTGFLCSILSLIFSFFIAFDKDVRVRIVAILKSKLQKQ

SEQ ID NO: 88 - protein, 131 aa, Bifidobacterium longum
MLGFSSSRYLAAPVMRLRMLSEARFGKIQLHTRREAFKYRINIIIQIDYRFHIHIINNLTTLNILNCGAKLQRPTHNLENISNLYCFCSLLFNIATILTLTSLSKAIKKLKIKDNIEHKNPVRKRENHPCG

SEQ ID NO: 89 - protein, 452 aa, Bifidobacterium longum
MTHDATDQAQYSTPVSRPIYIRHLDVTDRSDYLLYAALALLPVDGTVLGWYMPFWTPISPWLLMLYTALNWRLIPQVYRRFRTFFLFPLLLVALSSFGWFTVAFHPLPALWSLLGIGGALACLASLGIAVTIKHLDWRQMIRIILIAYWFAFAVGVVQFLSIKLDITFVRDWFSDLMSREYITADSAWGGNRPQFLFAEPSYIGMHLYGVLLPLMWLMRRRDRIYARRLRDLIIVFAAGSIIMGAGVRIILDTGVALVIAIIVDTDFKNHKQARLAWGTFGVMAVAGVAVALLNSRIRAILAQGPLLGDDSTSARISQTMTPLVALIKHPANLLLGFGSGNIVEANRQGTTAAYAILNGPDAKVPWWVWKSLTPTNVFTMSSYTSFITEFGLIGFIVLVSIILRHITRQHAWSKTTTCWLILTAYLYLQFEGYAFYVIPLLIWTSPKIEGRC

SEQ ID NO: 90 - protein, 470 aa, Bifidobacterium longum
MNKYKNLLLNTGLFAFSQFATKLITFFLVPLYTYYMTTEQFGVTDMSSTVIALLLPLVTLSASDAVLRFVIDDKKNQDKYISLGVGLIACSVIVVAVSLPLLDLQFLGGLGKYKGLFFLCYVVSACQYFCGLLARALNQLKLIPAASIISTLVTGVLAVLLIAKMGYATEGYFWSLIIGNACGALTFVFAGKQYHHIQFIRSSMDLILLKKMLAYSIPMIPNALFWWIGVSINRFFITGMIGIGASGLFAAAQKIPNLLNTFSGIFQQAWQLSAFQEFKKKDISGFFATVFKLYHGGIAIVSTGIIALAQWLASFMLQKDFYYAWPMISVMILAFYFNILNAYYGTIYTSAMKTKHLMTTTVAGAVSSVICTWLLIPIAGIYGAGIAMVISNALVLVLRVITAKKILVFKVDWPSVIVTMLLLISQCVVSLIHWPSYLVVSWVLTIAICGLQVFSCRSVMSRAIAMVRHR

SEQ ID NO: 91 - protein, 241 aa, Bifidobacterium longum
MLVFDGIVTIEVYLKSFLAHELSVYGGEFGYMRQEGLPKLSYDAHLECLASLRTTFMKSSLPYLRHFRNTYSNPLPPYWMIVGCLSYGTLKGDFYQGAPDSIKRKLATRLHIFNPNPNPEVRGDAKILSNWLETIRQARNMTAHHDRFWNETSTRIAPKLPKHRSGAHAQDWWGNDWDAFRKASGPAAFLTMENFLLTQIDGPSWREKFVSLMNRYPQIPKSDMGFPDNWESLPLWQGLSL

SEQ ID NO: 92 - protein, 77 aa, Bifidobacterium longum
MLFLIRGNDKWPKCFAEELRLRQFIETLKLLGKIILIMMESFRVEICNDSLRMLTLITSLSFTKIESNGIYFFPLIS

SEQ ID NO: 93 - protein, 414 aa, Bifidobacterium longum
MVKNVGSVVRWQVAKLALVQGMQAMSSAFFTCGVVFTGAASSDSLGLALVLMFKTLPTLVMAFLGGVLADRLPRKTLASGMLGGLAISYGVGTWVVQLSGLGWPVQAISLAAGIIGAVGSPALFALLPSIAPPEDIVRANGLIRTFRNAGSVVGPLLGAWLAQLISPSFLFLDGAVCLAVSMPLVLSLKLMPSCDDEANNNDDDASMISALRSIPSLFHTYIWLAVGVPFWALFLAVQSGATDVTMPLWVVQESGRGAWSLMASITSSGYICGSLIALKLERPRHMFSKSVLFGALAIMPIFVVGTLDIQVLWYVASFVAGLGLELSGVFWGSTMQTCVDKRHMGRVSSIDYAISFGLIPLAYGLYGFTGTIHAAVVLTVSSSIMIVLVIIVFPFCYLIDHRSNSSGIINSSDV

SEQ ID NO: 94 - protein, 243 aa, Bifidobacterium longum
MLHDSVAQCLTSIRLIAQRQSNDPQNDAWEKIDKIARKGLEATRDIIDTMIQDNNENGIPIESSEWWLAVKRLTDECDYILHQHGFTGTTEIINGGFTVQQQVTVKNIDILHEVLRELCSNIIKHAPKYSEFQSSIALQKSQTEIVMSNSMSTIPDEERSGRGLESRRRKLNLIGGEIDHEVDGDTWIVYARIPLTMVSPDKAATESMEDNEHEKYSPRKRGKTRSVECQREQMARKEHKAQK

SEQ ID NO: 95 - protein, 95 aa, Bifidobacterium longum
MRGNHRAINSNPMWMKPNNISYADVVGLSVQSIIEKNKESNTIINLNGCFLMLLFIITCPDSYCLAHCFRFELTTSSIKICCCTSLSNLHAIRGF

SEQ ID NO: 96 - protein, 233 aa, Bifidobacterium longum
MNNEPREEDSSVECYHSTAVASAATHIALLDNDAIVLKGLQQIIEYNHLGSITWTTRSGREAVQRCSSAVDTPHLLLFDMSLDGMSGIDVCRQIRKRSASVLLLGITAFPLERYISRLIQAGAQGLVAKDEERQIAEVTRWVLNKGGCGNGFETARNAHMRLKHETNDIRMLLSDREEEIMILLSKGLSISEAANRMQIGQASAATYLNRARRKLKAETVRQAVAIWTGDYEQ

SEQ ID NO: 97 - protein, 205 aa, Bifidobacterium longum
MIRIGLTGGIAAGKSTVSTRLRELGAALIDYDELARRVVEPGGVGLRRIAECFGPDALTDQGRLNRRWIAEHVFAGPDSERMRRKLDDIEHPLIYDLALSRERQAVADNPDAMVVHDVPLLAEVLDAMPMRFDHIVTVEAPEQVRVDRMVSTRGMTRDDALARIRHQSSPEQRRVIADAVIDSTQPMGRMLEAVDALYEQWLAES

SEQ ID NO: 98 - protein, 211 aa, Bifidobacterium longum
MSALLITHNLTYRIDDRTLWEGLNLTFSPGDMVALTGESGCGKTTLLNVLGLLEEPSSGTITYDGQTIASRKGRRLMHRNVMGFMFQNYALVEQWTVNRNLILALRSVGIPSADRSRLIRRALRAVNLTGYGNRPIYTLSGGEQQRVAIARLLIRQSLRVILADEPTAALDADNRAMVMRHLRDFADNGAIVIYTTHNEETAALADRIIAL

SEQ ID NO: 99 - protein, 656 aa, Bifidobacterium longum
MHWTYKIASILAVALITLAAGFYAQNNEETYPSGPSYDIGISDAHGQPLSTLTGLAATHHVVLARVSYEPDGHGTNRRVISIFGALDGNGMHANSPYPDYGFEPRTRVQRGDRFTDPLGRWLLYGSPRDTASVAKSIRRQGFELNRATPIGITQITKQFFSNSIAQVIFAGLAVVFVSGALSVSTASRVCAIQALYGMRTTGIIMRQFLRHAFFFIICVCIGWMTWISIGAIFWPFASPLGFAGQVFLSIIIATTCMALVALSLSIALVRVLVPNTLKLIQGKRPLRFLMASGCIMAIIVLALSSASLNVTNFKWRQSQTLKTTLEHQLSPNDGFQLQLWYSSDQNRARSMPSWNDFVEQTSQSEHTRFASFRLGCTWVDSSQDPQPCILMDSRTARLHHLIRNNTTLARISVIMPENEQWNSESITNNVLRAYSFEQSLAAEEGKSLPAINRMSISIESRPRDAVLSAFDTPSNTDGLSSVPVVVIDPSLLSGDTTTSMVSTGGMTFDYSSRHQLLEILREKGVDSLVASVVNRHDEIQTRLARTTQEMNYFSITACISITCLLGGAIMVALTLCTLRRQIMFVEYMHGAPSYLRFQSILFLAAALCSASLPIQLLIGGYNAVSTTSVSLLFIVVSLATTVLYDSRLRADSIKHP

SEQ ID NO: 100 - protein, 221 aa, Bifidobacterium longum
MKNGRPIRLALVDNDRCSAEMMALLIGRTIPEAHMLWVTDNPSLALERCLFDPRKPDILICDLMMDGLNGVRLTERIRQRNVQVGVIVVTSYDLATYGEDIACCGAQALISKRDFAATIREAVKSVSDGGTYPHGWGLHSLEETLHTVDAAQPEADGARLFSDRELAVLRLYAHHVPTVEIARRLGIGVETVYSYVKRAMRKVGVTRRGELLDYCERYHVL

SEQ ID NO: 101 - protein, 411 aa, Bifidobacterium longum
MSCDGSRFLRRRFRDFGKSWHGEKIAALACALCIAADTVIESCINPVWDAWALWVGLLFVILSLLCVAFPFGGNIALAISWCVVFPLPVDLSMSVSVVIAEPLIVLSYQRIWCGVVLAVAVTVSRVAQLIWQYGLPMGWDAGALVSVVPWTVMPALACVGIGLLLNWHHREGERESEARGRAESLDLAARLHDATTNDLSYLIMSIDRIMSEHPSQGESMDLPLLREVAQRALDQTHDVIAVLAKHNVGTARIPRCHARRSGEAIVPIGAGRFEAEIERHRRELATLGFRGEVVVSDPFDLLSRFDEKTIQLTRSLLEETFANIAKHADREQGYVFAIQVRQDGLYVSVADVPAKMADGALAESPRTLGMGFGMSHLRQSITRSGGWLRVQEEDGYWSCLAYIPIYHRDTA

SEQ ID NO: 102 - protein, 186 aa, Bifidobacterium longum
MLRDGSSSRSVPESGGRSVMAVVATLSGAGVSFAPLYERIWGGMMLHPNCPAIGRMPYYLSGQATDDYGVCVTSPLWFMTVTALSVVAILCLTVAGVQGFSRHRLCSRCPVLVRSHVLFTYSYNTGRHTDFCPALSATDNAGLVAYCIGREALGLLTVVLCVLPLFSLLSAIMAITLAAHDATPPA

SEQ ID NO: 103 - protein, 665 aa, Bifidobacterium longum
MFTAQRETIWRVVFPSNNVGMDEEFRIAAAPIYGSGYDASGGSLSNAELHKEITGRTSLEVPPEHTYSTASYYNAFPAAYWAQWTNVQQVVLNLTVAGEGSVTVHRSDADANDYIVAKKSVNATATSPQVVQIPVPIYGMAKGGWLWFDIEASADASVTLSDASWQTEVSAKRNLTASLAITTMNKPEWCIRQFNLLADMADMNLIDAVYVVDQGSNLVEEHEGFAAAKVKLGDKLRIIQQGNVGGSGGFARGMYEVEHHGESGYALLLDDDTVLEPESVSRAIAFANHCEKPTLVGGNMLFLSEPTRICALAEVFDPQTISWGTAVKESRYDDLASTSFLDKQYLHRRVDADYNAWWMCLIPTEVIRKIGLSYPFFIKNDDVEYGVRAQRAGYRTVTVPGVCLWHQSFVDKDDQLDWQAYYHIRNRTIMGLLYANQQYKRNILKEMVRFTLSATAKMRYSAVALHQAAMRDVIAGPEHVGTILETKLPEIREIRSGFADSNMVPVEDLPDTLRAQDEKFAHLHDLSRAEAILGIGLHQIMPPRANRSAVIDGYMEPTKVHCLIDNGHSTHTVPADQLDSLVLVADDASDHWRALGLMDSAVFVDPDRRKGILLTRQPVRAITGFIRACGLYVKVIANWRTYQRQYRKAFATMVSPEWWQRYFTK

SEQ ID NO: 104 - protein, 632 aa, Bifidobacterium longum
MPSIRFANVLMEITPRALSYPTMYYHTNQPVRVNPDTHEWFVEGAGTIDFTTYFNSLSTMKLLKYTRATGFHLHLEVKGNACTITQTKAYRLSSSPEIDPTVFAKVQASNKWQSIDLDLTVDENMVLAGFQIETTGAIVVRDAYYTLDIDGELTDIELSLSTTTFKKESYITKNIELVKKEILGSDNDIAKHFRMHVIDNGCTLPYKELSTDKVTISPNENVGGAGGFARGMIESMEQDVPATHVLLMDDDVEVSPESIMRTYNLLRIVKPEYSEAFVSGAMLNYEDVQDMKEDTGFIDPQIGICVAAKIPLQVTKFVDIVFNEVYDENLRVGDGRRYAAWWYCCIPMSVIKRNGMPLPVFVRYDDVEYGIRCNPTFMTMNGLCIWHSKFEIRYNAAVERYQSIRNGMIAQMTTGLAPSIDTFLRELHDQVDLELKKFNYTDAELALKGFEDFLKGPDFIKQPIVQEKFVQANQEKEKLVSFPELQQMADDMGLEGFDVSKLTRQEIDNDKPRSIQQRAFDFLTINGQRLLHSSIHGAKGTPGKRYALISSAGWIYPAGSIHGENIIIAIDWFNRRGTIRTKNLKQYNAVTKRYKRDLAYFKKNRERLSAEYKAASKELTSIQYWKQYLGMK

SEQ ID NO: 105 - protein, 130 aa, Bifidobacterium longum
MRRVITYGTFDLLHYGHINLLKRAKAYGDYLIVGLSTDEFNAGKGKKAYFSYDQRKELLESLRYVDLVIPEQTWEQKRNDILLYQVDTFVMGSDWAGKFDDLSDICDVIYLPRTPEISSSKIKNDLEHRN

SEQ ID NO: 106 - protein, 383 aa, Bifidobacterium longum
MSFLNTIAKHLPASKRAVAQTLNEVKMLREHVDVLYGQLYARIEQADYGINDNLNYKVDTILTPHLNDLGTALDAHDAHMKIFAWENYRHKGESLSAAKQRFFMSLPPATGSTRLLQEGCAQLMTEFDQLCRDNNLPYWLDFGSLLGAVRHHGFIPWDDDTDLGMMREDIDRLQGIVQHDSRYRLSLVYDAIAFCRQIRFMSSDTSNPCFVDIFIYDYTDSTAIEVYDRRQHIRTELLDALRQSQFRAWHDLVYLSETSDGAAEIQQVFSRYQREMEESGIVVSKENASGIMYGIDNVDNSSVRLYKLDDMFPTTCLTFEDHEYQAPHTPMTVLTRNYGDIYSLPRDINSHFIHVDPALLQQDNVQESIQDSLSEIPISKNEE

SEQ ID NO: 107 - protein, 568 aa, Bifidobacterium longum
MLRELKLRLNNKNISVWVFAFIIMLLVLIGRAVDDGSQGGSKRSLAYAAVFGLLSWLIAYLIFHWFDCLTGSRVENGDSASLPLRQRVMSVDYWAATQDAISFTTSIKKYGLVCLIGVIFCWLPWVITCWPGVMRDDTIAQFMQSSGYHFYYTQHPLFDTLVFGFFWELGFALHHVLLGLGIYVLVQTFSFAIGVMLVLCYLRKIGAARSLLLAIFLFFAFCPAIVGAVPTMAKDSLHTVFLLPLSIIYVEIFLTRGKVLHRRPVCVMLVLLVALCMLSKRTATVAILCAFCVLVASVKKNRLKVVASMIIAMVLAQGIIEPALVRVTHAEVSPGKEVMGLIMMPVARIQSISPERISPQERSALSSLLNIDKAGKTYTNYRIDETSWTINNEASIAQKIKGIGAWVSLGVHNPGEYVKAFGNLMLGWFYPQVGVFYGSNSDGLFSDQYMIQWDSFVRPPLSAENVLHDMRGTGQKSSLLMRAADAGQQIAINPILNAYAYYATYIPLLLLIYGMSRKRWIAVGAGSLLGFNVLVLYLSPLVFAWYLLPVTFILPLFFGITGCIAEKQ

SEQ ID NO: 108 - protein, 126 aa, Bifidobacterium longum
MAKKMVMPVAWAQDVDCWLETLKAAGFSDDTVRSRRYKIARLCRELPSPMETTGDQITRVFAAHDWKPETRKGYRNTIAGFYRWFYETGRRGDNPTAKVPKVKKPQAHPHPCPDKYMAGSSDRCNT

SEQ ID NO: 109 - protein, 137 aa, Bifidobacterium longum
MKKIIAAAVTVTTVLSLAACGGNTAVDKSDCLDVPQDVLNVVASGSDSSGLKPETGKAVKGDTEGTYWLAMKFTADGFNGDTETGIWLVSGLDAASAAPVMSVDGFAKQFTHWPTQINGTELNGTEEKAKAAAACLA

SEQ ID NO: 110 - protein, 102 aa, Bifidobacterium longum
MAIQGTFEGFSEIGNRQGFMETRTRANLKTFFDGKTVTEAADTYAALMTAIAHNIDSYLTLGKNISTLADSYNNAFDHLRELYPEAPELDENLAALLTEAKA

SEQ ID NO: 111 - protein, 55 aa, Bifidobacterium longum
MRPRKGPIVRGIRTVLAAPFAVLAFALATVAMFSARAAMWISAGYRGAVKVEAEL

SEQ ID NO: 112 - protein, 63 aa, Bifidobacterium longum
MPVFTKKSHNHFDVDHFEVDNSFGFRITVDGDYFAFAGMSLGDLVTINQHIAEAIRKGRRNAL

SEQ ID NO: 113 - protein, 83 aa, Bifidobacterium longum
MYDVSVTKQTRPEITTGELIKRLLAFNGLTQQDMADAIGCSRSSVSQKCAGHVILTADEIAKTADLLNVSADVLLGRKPLEVK

SEQ ID NO: 114 - protein, 30 aa, Bifidobacterium longum
MYLYEGKALCNQFFSGTQPTEKYAIMNLDI

SEQ ID NO: 115 - protein, 145 aa, Bifidobacterium longum
MNDYADSITAERSRKVIGGYGDHEKIDSGFSFYELGPVLFDADGELNAAVPAEEIRKYIWYSETKAPYVDMTAEHPYLLGVLGETVYYLAYKPDGETTLGPRLLRLVPRRGAPTVVYADRCVFDDDKLNELNVVFKQIPRQIARI

SEQ ID NO: 116 - protein, 226 aa, Bifidobacterium longum
MEGSGMKPNTYTLNIDQWKFIVFTDLDRMDRTSFVSIAPGIAVRADYRIRAMEDRIGKYDIRLHMGYSEEEQRIVLRNCEIGTTRELKIRDIARLPIEQIIRSYRPPLWSYEITDTGTNIFGPLPDWEHDVLSSVDFPTLRKQGPTPDTLKWASRVYSVTQLNKGPATKRLTEVFGIPLRTASHWLTLMKERVPESVSMRLPSPITIHDETKPDTASGTALKKLLE

SEQ ID NO: 117 - protein, 64 aa, Bifidobacterium longum
MTSEHCNVTDGFSDRDRELLAMFDMTEEQVREAEMIAESETIPDGLVGPVYYGRHHTDASRLGS

SEQ ID NO: 118 - protein, 45 aa, Bifidobacterium longum
MFRSLGYTTEVTPASRDGGYDILLRGRDGVMSIVECKCYAHGATA

SEQ ID NO: 119 - protein, 130 aa, Bifidobacterium longum
MPVGQHAVHLLVRVRRYECMACARSWTDDLTHMADEGRRLTDAAVWWAVAEVVLKSKSVLACARDLHCSWGVLNRAVLEKGADVLAADLRRLDGVEAIGVDGHVWRHTRTGGRYVTVIVDLTPRRHGRPA

SEQ ID NO: 120 - protein, 49 aa, Bifidobacterium longum
MSKSNCEPALLSSLETLQHNLQDAGMLRMKASLYSEAAVRDVLRLLEAK

SEQ ID NO: 121 - protein, 447 aa, Bifidobacterium longum
MKLQRKALKTLKQWKTTPDHKPLLIRGARQTGKTWLVNEFANGQYDSIVSVDFMQRPSLSGIFEQDLDPQRIIRQLELAANQRILPGRTLLFFDEIQESPLALTSLKYFTEQAPDYDIIATGSYMGISKHGKTSFPVGKVTMMNLHPLSFVEYLDSIGQDMIADTIREGRFEDIPQALEPQMNDLLKTYMWVGGMPAALSAHLDNGIPQDVRAVQQDILNAYDLDFSKHAAYTLGERIRLVWNTLPSQLAKENRKFVYGVVRQGARAREYEEALTWLTDYGIITKVPCLDALHIPLTGYESLNTFKIYLEDTGILGALSGLDVNTLVNKSKLFSEFKGAFVEQYVCQQLVAQGIKPRYWANPNPQGNAKIDFVMEQGDEVFPIEVKSSSNIRARSLSYVCNRYGLHGIRIGEIGYRKQSWLTNIPLWCVDGLGEYLKRQIEKSRAEA

SEQ ID NO: 122 - protein, 440 aa, Bifidobacterium longum
MNETATFDAFFESIKLDSLNEYQNVLDCIGKKLNDSFYHIDSKNEHLIIVGSIGRGTAVPGTSDLDVLFDLPEDVFHTFDSYKSNGQSALLQKVKEAVKERYPKTDVRGDGQAVVISFESKNFTVDLVPAFRQTDGSFKYPDSHNGGSWKTTNPIPEQEACTTLFAQTDNAALHICNALRIWKNNVGFHFKGLLIDTLVGKYFDQKNSIPLNSYDLFIDVFENLSLVNRNQSYWHAIGSNQQVTNDDKGAFVPKSQKALNILRAASSESDREEALIKLFGKTIAKCMVDSIHQENERKFLKKYSITNNEEFIEDLFTIDISNYLEIDCKVTQDGWRTKSLRDMLSKHLPLLPRKQLDFHIVRCDVKSPYEIYWKVRNCGEEAFKRNCIRGQITEGTLDAPLREHADFQGPHFVECYAVKNGICVARSRIDVPISETGEII

SEQ ID NO: 123 - protein, 191 aa, Bifidobacterium longum
MLFNLCSKKEKHMTEKTQIINKRLDQILYGTEWTHKIHEKVADTFEITDRLLTFISVLTTAFSGSGILAAVFSNNQSLRIAAAILAAISLFSTLLTKSFRFSVRATEQRHAAREFLSIRECIKNLQVKINTDNISVDETLTEVFQLSDAYTSACIKAPSTNFFAKYLAEKEFLRSSSELTSSYKKERNTDE

SEQ ID NO: 124 - protein, 204 aa, Bifidobacterium longum
MSFTLPSAYSLFSSRNEAKSCSPPVVGDQPFGRAARVPDAGQGQGHPAQLPAGLQTGPPAGQFEDVGLHVEQAPLDPRIRPRGLRGLEDAAPAVAYEHVGRRDACHQALPCRRFLAFGDMPADHVPAGHRDQDHRVAVQVDAVHMHHMMHLVHQRHGRPQAPHELAPAAQRACRQPVLGLRLLREQPVQTAPQITGAVVARLGA

SEQ ID NO: 125 - protein, 165 aa, Bifidobacterium longum
MVRTEDACEIIKYALQNEIKVYLDGGWGVDALLKRESRIHNDIDLFVELKHYHDYIYVIKQHGFEEVNTDYTTDGHTVWKDDKQRIIDLHCFEFTDDGIVYEGDIFPSKTFSGIGKVGDITVSCIEPLSQVMLHLGYEHDKNDVHDVMLLCETFQIAIPDEYKEK

SEQ ID NO: 126 - protein, 514 aa, Bifidobacterium longum
MLQIKKLNLTHKKDLRIILNDFNLVLNDGDKAVIIGEEGNGKSTLMKWIYNPSLVENYIEADGERIMGHERLGYLPQEMLDEDKEKTIYEYFSEEEIFWEKTPKELSVIAGKFGMKNDFFYSNQTMGSLSGGEKVKTQLMRLFIRDVSVLLLDEPSNDIDIATLTLLEKIINDWKHIVLFISHDETLIERTANMVIHIEQIIRKTKARYTVAKLPYRRYVEERLHKFEIQKQRALSDRREKKIRDEKYQRVMQSVQGALRSCTRQAPSVAKNLKDKMHTVKAMERRFEKEDENMTQMPEQEEAIFVKLGDENSHIPAGKTVIEYELSKLVTPDGKRILAEGIHLKIKGSEKICMIGANGAGKTTLLKKIAEELLNRNDIKAEYMPQTYEDLLDLDVTPVDYLDKTGDKEERTRIRTYLGSLKYTPDEMEHPIRELSGGQKAKVLLLRMSLSGANVLILDEPTRNFSPLSGPVIRKMLREFPGAVISISHDRKYIEEVCDKIYQLNPNGLQLIGD

SEQ ID NO: 127 - protein, 125 aa, Bifidobacterium longum
MTKTIFEEMGGTYRQVGDYLLPNITVPAEEEIEPIGLWGKRHARHLKEHYKVLYMNLLTSGKLHSYLAEVDKQAEDMFLRLVKEYADRQDVTEQLKKDNPYEWIGRMNNIQACVREVVGTELIYT

SEQ ID NO: 128 - protein, 386 aa, Bifidobacterium longum
MQRTISAMVGKGSVNHNSRKFRAENVDGTRTHLNIDYCNENIKTVYHELFDEALERYNAKQIRSDRKIKDYYEKIRSSKQEKPFHEIILQVGGKGNMNADTENGELAKQILDEYYQGFQERNPQLRVFSAHLHMDEATPHLHIDFVPFTTGSKRGLDTRVSLKQALATQGFKGGSRGDTEWSQWIQSEKEQLAAVMERYGIEWEHLGTHEKHLSVLDYKKQEREKEVAALGAKIEQKQIEFDVLSERVLNYDKAKDELSNLEIELDTAPKYQLPEPEKFMTAKAYKTKMAEPVVRKLKQLVKTVLARCFEGWDNYHRLNTANAQLYRTNQRLEKVNERLTEENKILKAENKDYSLLRKVFGRKQIDDLLEQARTVKGRKRDNTRSR

SEQ ID NO: 129 - protein, 384 aa, Bifidobacterium longum
MENKKEMTIPNVSAATDAEQSLSKCTDNSIVNQDTDFKGYEQSFEEMQREILRQLDPSYLKTVSMTTLYDTVFEVQTPLIDGLLQRGTYLFVGSPKVGKSFMMAQLAYHISTGTPLWEYKVRKATVLYFALEDDYPRLQKRLFQMFGAKETGNLYFATECKTVNGGLEEQIRGFMREHPDTGLIIIDTLKRVREAGGADYSYASDYDVVARLKALADSYKVSMLIVHHTRKQKSEDIFDMISGTNGLMGAADGAFVLSKDKRTSNNATLDVAGRDQQDMKIHLVRDSERLVWNFAKSETEMWKEPPEPLLEKIADTLFSESDRWEGTASELCERLAVDIKPNVLSLRLSINASRLFRDYGIRYQNSRTHDGRKVSLWKETEQTA

SEQ ID NO: 130 - protein, 111 aa, Bifidobacterium longum
MSAKNRDNKNRWRNITVGFRVSPEENELINRAVALSGLPKQEYCYRRCLNQDVVVQGNPRVYKALKTEFATVLAELKRIEAGKGVDDELLSVIELISIILGGLKGEDANGE

SEQ ID NO: 131 - protein, 349 aa, Bifidobacterium longum
MARYVNWKGERKQKCKRGFATKREAQEWERMFKLQTSSDLDMSFEAFTELYINDVKNRLKENTWLTKEHIIRTKILPYFGKLKISEISTKEIITWQNEMLAYRDEKKKPYSQTYLKTLHNQLSAIFNHAVRYYELRSNPAAKVGNMGREEHKEMLFWTKEEYKKFSFEMMDKPVSFYAFEMLYWCGIREGELLALTPADFNFDKETVTINKSYQRLKGQDVITSPKTKKSNRTIKMPKFLCEEMKEYLGMLYGLKKKDRIFTVTKSYLHHEMDRGAKAAGVKRIRIHDLRHSHISLLIDMGFSAVAIADRVGHESIDITYQYAHLFPSKQIEMAEKLDDLGKGDFENVS

SEQ ID NO: 132 - protein, 62 aa, Bifidobacterium longum
MENRFIRAEDVAQELNVSKPYAYKLIRQLNEELKAKGFITIAGRVNRQYFYERLYGAGKGEM

SEQ ID NO: 133 - protein, 176 aa, Bifidobacterium longum
MAISERIHFFRLMRGMTQKYLGTAIGFPEKSADVRLAQYETGTRKPKADLTNALAQVLDVSPQALDVPDIDSYIGLMHTLFTLEDIYGLTVSEADGEVCLKVNKDKGREAYELLKMLYAWKEQADKLSSEEINREEYDNWRYHYPEFDTTQRWAKVPSQELSDALVEAFKDHLKDK

SEQ ID NO: 134 - protein, 90 aa, Bifidobacterium infantis
MIDVPSDAPNFTFTVANESPSRLLTWPEASLPATAVTGRIKASTRRARPWAAPVGRVVLVARARMDLDERAPAVGCHADFGVPPSPEDAD

SEQ ID NO: 135 - protein, 220 aa, Bifidobacterium infantis
MNRAGGSSGSLFPHWTLRLPLRDQKRMIDPDPLFLLYWLLRGPSCGRDCGVVPGRRRATRICQYARNILQACSGKPAGNAVSWHLLMHLADMRALSVGAGPRFDSGTTPRLRYPTRWPSIRRRRAKYWYCTDSQTAQRRQRTNLWRNTDSGKAYRRQIARLWHTTDATRPTTIGNATNIGPPPTLASLSVGAGPFHESVTAPMVSERRHRDAVWISTDAA

SEQ ID NO: 136 - protein, 402 aa, Bifidobacterium infantis
MSATAPNYNHTVTACFVGYITQAVINNFMPLLFVTFAATLGIDMARLSALITVNFVTQLVVDVLAGKFVDRIGYKPCIIAAHLAALAGLLALGLLPTRVPDPYLAILAAIFLYALGGGLIEVMVSPIVEACPSEHKAKAMSLLHSFYCWGQLGTVAISTLFLFAFGTGSWPVLACLWAIVPAIGIAMFAGAPMPRIVPEGTATMRFADLSKKPVFYLMFLMMLCAGAAEQGMSQWASAFAESGLGVTKVIGDLAGPAAFALMMGLSRTIYGVLGHRLDLTAFIASSSVLCVAMYLTAALTTAPVLGLLACALTGFSVGIMWPGTFSMAADAMPGGGTLMFALLAVAGDLGCAGGPAVVGLVASANGDSLKTGLLFGSMFALVLLACVVAARKTVVGEREPLH

SEQ ID NO: 137 - protein, 306 aa, Bifidobacterium infantis
MTSASDRYQEKSDKKEHTMRNSSTPPLLRLEDICVKFGFVEALKSVNLSIQRQEVIAIVGDNGAGKSTLIKVIAGFLQPGFGHIYLNGEQVTIPSIREADRMGIASVFQGQEFCDNLDVASNLFLGKEINQIGIRDDDSMNSRARSVLKTLSSAIRVGSPIASLSVGQRQTVAIARTLLNDPQLILLDEPTAALSVMQSAEVLAYIKRLRSEGRSVVMVCHDLPDVFAVSDRIVVIRQGHVTGVHRTVETSYEEIIAEIAGVTTEHEYEEIAENPKFDSMVRQRKLIDRTISAAVSHGTGHDSPLD

SEQ ID NO: 138 - protein, 389 aa, Bifidobacterium infantis
MATRLFGSQTSLREANRANLLASIHKFGAMTQVELAEVTGLSTATVSTLVHQLVDEDQLETKSTVRNGRRATLVTLARHQGLGVGLWIARRHLTLSIVDFSKSIIAEHTLPLPLGHKADTTLERAMLLINETLSSIDAEASELVGIGVAVAAPVATSDHTIAIPGILPGWDGVDITSPLRTAFNVPVYVDNDANFAAYGESRMGVAAGKRNFVYISASDGVGAGIVINGEIMHGVTGLAGEIGHIQVDPLGAICSCGNRGCLDTVVAENRLVQLLSVTHGNMTLDDLVSFANEGDPGCRRIIADAAVRIGQVAADLCISVDPEVIVLGGKLAMTGDVFIQPFNEALQRMLFPDAVAPIDVLVSSHPDDNCALGGALCAIEFSVRNDVSQ

SEQ ID NO: 139 - protein, 385 aa, Bifidobacterium infantis
MKFAKKIVAVVAGVAMCAGLAACGGSRSGQATGGDAKIEKGATIGISMPTKSEERWNKDGNNLKAKLEKAGYKVILSFADDKPAQQNADIENMVNNDAKIVVVASKDGTAVGPAVEKARDAGAKVIAYDRLIMNTDAVDYYATFQLEQVGVLEATYLIDQLKLKDGATGPFNIELFTGSPDDNNAKYFFKGAWDLLQPYFEKGVLVSPSQHGQGGVTKDFTVEDWQKISVMSWKTEQAQKDMESILDSTYAHGEKLDAVLTPYDGIAQGVINAIESKRPDMKPGTDSWPYITGQDAMEIAVANIAKDKQGETVFKDVNKLADAVYDMVVEIAEGKEVSGLNGKFNNNNIDVPSKLLDPQNITKDNLQDLVTANYITQDRFDELTK

SEQ ID NO: 140 - protein, 84 aa, Bifidobacterium infantis
MKLTARSTSRMYALTCLACVIWLWQSLVEANNDGSLFNWATIVFSLCLLVVIGWSGWNAVAGWNAKETEAATAGAKDDEGSTDR

SEQ ID NO: 141 - protein, 164 aa, Bifidobacterium infantis
MCDSRTDTKTGLLPVNEVRSLLDVCWKAKAITELMPALPKGLKPRYVHVIDAVWHINETNGQEIGTARVSDVSAFLGVTTPSVTKLVGEMVELGLVVKHMDAADRRAVTLTLTERGLDIRRVYVEEYHAHLSQLLGGLTVEQCETTVRTLTEALRLMQQDANNR

SEQ ID NO: 142 - protein, 166 aa, Bifidobacterium infantis
MHRIFRETLCSRQRHHPTHRNFPMTPHDQLGTGPTAESLSTSLSTAITNIACIAPPRHRSLINQHREHIHRQICDAFLLEHVGFREHHRLVNGVAVSAQRIVKRRMRTVISARLNLQGQNITIIGLYQEIQFANSFFRKVIQIGESMRGKFLSHDILIDSPMFIAA

SEQ ID NO: 143 - protein, 73 aa, Bifidobacterium infantis
MNVVTQRISALVKDEGLTCAQLGSLLGLSKTSANGKLLGRIGWTTSDIVVLSEHFHVSTDYLLGFDADHEEVA

SEQ ID NO: 144 - protein, 122 aa, Bifidobacterium infantis
MAGGIYEQSEWPDSRWDCVTVCDGCHTVVWSTFWDDYDSAARDAYFARNGWRNYCVPGDTEILELCPACAVRALRRSETRGLADSWLRPTHAYTHAFREVDAQLSARERMVAGLLLTEGRAS

SEQ ID NO: 145 - protein, 237 aa, Bifidobacterium infantis
MSNEVSCDSVARRVNGENPLDVLVGVSTPYGPLLTDDYGWSIRFAEYGDGPCRFGDRPIQVMKPYEWLADRVYVFDDEAVAPFKDDVARFVVEACRCFVGGDGDHDRSVVLCREGVAEQLDLSSDAFTPMVGSDGNAVSFSYQPCDRLRCVCSTNPQKGLGVFHVWTERGTTYQAVLGPCAYERRPEKALTLPDELWSRNESWMRDFFEQETSDFLCLGVVSRRTNIRFVEGGGAMV

SEQ ID NO: 146 - protein, 63 aa, Bifidobacterium infantis
MNDFTKAFRMSCSVFPECNRDFQAPVWTFPVVAAFARHNGPGSVDSRRLASMMAHPSMEGRLA

SEQ ID NO: 147 - protein, 376 aa, Bifidobacterium infantis
MSNIALSWAFKCHVGNASAKAVLVYLADRADDDGTAAYPKIATIVNVTELSERTVRTALKTLQERGFIRRGDQRYARLGKGGRNRLPQYCQIVWDLAVESDPSTLEWIKETHTAEHDPKTMGNTVDPAASTIMENGESKDVTPENAGTKPIPSTANLAGLENDPEPALQISHLQHCESCTPSTANAAGLLYKDKTLQVNPPSKPSFPSAPTGHLPASGATAAEKNKTEQLDEDDTEIAEAAGRVLASLGAHRSMLGLATPSPTKADRKAIIGLYRRLVDQGAQWPTLVMVGAIGFAMNGDWWPKRIRTGRALARHWDELNDDMILAAGRTDGDAHAQTVPAAVPEPDAVPWLPDWAVETLAELDGQDATAGGEATA

SEQ ID NO: 148 - protein, 84 aa, Bifidobacterium infantis
MTTRIDCSEAGFSEFLLANPQLDGHADLIWQLHAVYWRNKRLGHPKAVGLLIQYARAWAARNPGETAIGRLQARKTPMTQGRRP

SEQ ID NO: 149 - protein, 165 aa, Bifidobacterium infantis
MNGRDMMPACARIAAVDPAMADRMWNTTTDDDGRDLVDERLRGKGRVLCAACPMRLDCISRALVNGWKDKAVYGGLDYASRWILARLIARDLHIADGGLHRIPQSRVRDWLAEHPDWAERMRRDGRDYWRRTKRRQRSRREYTHDDPLSLPTEPVPKGLVQGSLF

SEQ ID NO: 150 - protein, 223 aa, Bifidobacterium infantis
MNDKKNREPMVIALATGKGGSMKTTSAVFLACALVDQSRGEQRVLVADADVQGDAKDWWYKAAELDDPLPFDVMSAAPADITHLHGINGRLDDPVDWILIDSAPYGRALDESVNNADLVVIPSSPSRIDLDQAAGVKDLCDRRGVPAAILLCRTEANTTALRDALAWMDDAGIACFEALIPKRQDILNAKSTRPRGSRLHEYRDLAAELKQTMRQLKDKEEDL

SEQ ID NO: 151 - protein, 93 aa, Bifidobacterium infantis
MRNMNLTPRPRDLTALLNSNRTEPADTGRPESVETEIPKNRRTVKPNDAEGWVKTSVSLRASTRRRLKTWAAEHDMRIQEVVDAALETYLGLK

SEQ ID NO: 152 - protein, 158 aa, Bifidobacterium infantis
MFLKGECADFPDSWSDRMWGPDDLPNQRSQYDLRRAAVRICEACPVRAECLAFGIMVRDQYGIYGGLPLRARRQVLKTAQETGFRFDPDDPTAERRLARYIRENPEIVAAAREKECKRRKTEQRNARQQRWRTTTRSTGKAKAPAAATHTPPLQDTLF

SEQ ID NO: 153 - protein, 351 aa, Bifidobacterium infantis
MTTNTKKVAFVNLKGGVGKTTSAIEPVETPAENITEPGKDGRKCPKWLPPLIAAVCAVILVAAGIVGWNAYSGAKLAEAKEACAAAAAVRNNANEYNALLNGDAADAAAVKAEQVKDSKTVESLGKELKAMAPEYEGCVAEDAQGLDAATVKLNEQADWYETHEKSLSKAVRAVTESKAAKTLETAKTNLTAKLGEASKLLADSDGKVADNATRDALSNAIDAANGLKDGNDPAKIDGARKTVEDAINGVNASVQAKTDADAQAAAAAAQAQAQAQSAYNGGSSYSGGAYRRTEGSTSGSNTYRGTTSGGTGSGSTSGSGPAGGSAPKPNLNGSYGCGNSCTGKDDGYYHH

SEQ ID NO: 154 - protein, 1794 aa, Bifidobacterium infantis
MNFGRKMMKAGVAAVAAIATLGAGGVVASTAFAGGGGGNQPGVGGNMDVLQFWQYKDDTSGSWGPATSLDSVRAAMNNAGVALQGDGVTKAQAALDQARTECETGFRQRHPGEGDGDCRVVAVGAVPYISGRNFIYNGTGYYSPSLPGGWYDNWNKYVAPGTYQYGSTVYRTSYPFDDDPSNSVDAIMRRNVGASSKPSIVVIVLDKYQPAPPNYDLTVSTQAGGTFTQAGATGNVSDAITTSRGNSSISENVTGTITLHWTGLDGTTRTASKQFTQDNNTTQNVSFGFRDVDKTWKSWPAGSYYYDVNVPKQGKMKADAGHAGSADARESWKPVPTPPSKKLTNAAGQQVTSDAQQIASGSLYTAHITAQSNASEHFWLYDTIDVTAQKVLIGGTDRDDVSKVTVTDQDGNAVKADITVDDSQPGKRIVKAHVLNPASGQYTLNVPQSATPTGSDYTIPDDSQACWTGDEYGSTDKSHCQTGNSEQVGKVTPKPDKVWVLDSNGALNAEDPEHTNDKGSDNRTFVTGDAIGAVVNGRIPAHLLNPFTSYSITDDWTASAQWIDWNHKDQVRVYVDGKDVTDQFDITIDTAKHTTTATAKQSFLTKTAFGTADRKVKLYIGGIVKQVPNAQAAADQKKLTNKATETWNNESRPTNEPPVWVRNPKPDKVWSADQGQAANAEDSAWANNVNADTHTFVQQDDFGVTVNGLLPRNLARKMSSYELGDDFSKSARNIDLDSASVTVTIDGKDAKNLFDVHKQDDRVWVSAKQELLDTTYNQAADRKVRMTIKGAFLKDVLKAGQKVQLTNGDWEQWNQQTVPGNEPPVKEWSPNPDKSWIKLGDDGKWAAVVDPTGSNKTGADTLKFLDGDQVASVVNGVIASDLVKVTDIKLTDDYGQADYIWDLASDQSQIRVYEEDATTDAASSVADIANKGRDVTDQFDITVAGTKVTATAKPEYRAAQAGLKNPKQISLLLPGVVNFANGKGAAQVRKDFKKNAGDELTFCENPDGSKLTNKGSEKVNNESQPTNEPYICGYVPPVVKKVVAEGSQGGANNDANDKVVYPGQKVEYRLTTRPQLPSDLAYRIVSIRDTDTYDQYLEPDPQTLEVTDLATGDQLTTSDPQMGVEGDYTVAWDNANHQFTITYSDKYVAEHWQAGSHPQVQIRFEGTVAKDAPTDRRVNNQWMLTLNNSITPSNIVDNLPPKHDPSKKDNQSKEQGDPSISIDGKTMLLGDTGNYVVTLDLKQTNNAYRVWKAGITDDFDDEHLAIDGTKIEVLDSKGQDVTGKFNIQIKDGVAYVYAKTVDTWIPKKGVTVKGDPQPTDLAAYASSSKHDPLSDPSIDQNLLGQEYRIVMPYKVVKVEDGYTVRNKAIQVTNDLTRETNEVSNPLKEINPAKDVTIKVGGESIDGRSVYKDRTFLYLFDSSIIPAGRAYPRVDQWRIVDPLNTEYDQYTGQWAVYASRDLYRDGKVIAAKGDKLAGSGFDSSKFGGDLFDAAADANGVVTVEATEAYRTLVSADNSHENGWRAYIQCKRLKVSDRVENRFTEYFNDKEFESNIVWTRTPDMTPSIHIEKYDVASGEQAGDRDDVKDALKMAGDSQQIAFKITNTSKTDSSTGEGAWYLAKDLKMVDRTIAGEGDVTDLKYPDNWDTLVLKPGESTIITGTLKGVEQGGKHTDRVKVTGTPLVECPVTDQFGGQQSTDGDQTGDTKVDGDASDTTGLKQVKVGDRTLCEDTTVESNTDDWNGYRAKPLASTGTAVLGLAGGALAVLLAGGSLLVFRKRHRAQGSGRHTAANAGK

SEQ ID NO: 155 - protein, 91 aa, Bifidobacterium infantis
MADRQTPLTECPVDTSAPFGDGTSDDESGSKPEAKTESKSDDVVTIDGKDYCSDTKVESATDDWNGVAIAVARRRRKAVTAGAEHASDDAQ

SEQ ID NO: 156 - protein, 281 aa, Bifidobacterium infantis
MVDFIVGILNSIGSGVGDDLVADLLKTPAEYNAGMYQLSLTVARSAVKPIASTILAIMCVLELARVSTRADGDRELGVKLVAMAMFKLTLVFTAAQHSELMLQAIDEIGDSVLGGIHSAAPTTGASSGLGLGDSMRDAIDSAGVLGQIPCLILLLIPFLVSKGATIVVTVVILLRFVQIYMLTAFNPLPIAFIAQEETRQWGINYFKQYASLVFQCATLYLAILMYRTLVGGTLNPSKFKDGDSLSGWVMDNFTGLLLASVMLIGIVMAANSVAKKLFGGE

SEQ ID NO: 157 - protein, 798 aa, Bifidobacterium infantis
MFGKKKTPVVAPAEGGAEARAKARRKKATRLPKGVKQLIGYDAMLRNGIASLDDGRWSATILFQDINYQLSPESHQMEIIDRWAKLINSFEAGQSVQIASYTRSRGVREILADVMMDETGDGLDHYRLDYNRLAQGKLESVSRNTSTVKTLTVTVRESDEQAAVATLNALCNNLVSQMRSIDACKATRLDREHRLRLMAEVLRPGEEFRFDERRFEHQPGKPDTKDLVCPWSIDARNPTQLDIESLDSKYLHRTMWVSSLPPELSDQLVNDLTGLRARVDVSIHLAPMDRGESMTLVRRKNAEVKMQIMDQRRKNRKQGLDPDDLPDDLADQQEQLGQLRDELRSTNQRLVDSIIVIGVSAASQEELEVACRNVKAKVNAQSCTAESLKFMQMEGLTAELPLGNNPLPMKRTLTTNSAAILIPFTTQEVFEPHGLFYGSNARSGNPILADRRSHMNSNGFVLGTSGGGKSFTVKQEIAGMFLNRDDEVIVIDPEREYLALAAAFGGQIIQISAGTGTRVNPMDIVLEDDSASDPVKDKTNNVVSMIGALIGGIDGLDPLQKGLVDQCVSNLYTRYRNQGGGVVQPTLQDLHDELQAGGDQVSRYLADALNPYITGSMSGFNGQTNVDLSNRFTVFDVSGLSGELRTFGMMVVIDQVWNRVIRNKANGRRTWLYVDEFHRFFSNQYAAAQFKDIYKRARKYGLGVTGITQNVEEILDLQDAREMLSNSDFLMLLSQNSTDADALCELLTLSEEQRQYFTGVLPGQGLMKIGSAYVPFDGRIPAGGDLYRLYSTTFQEGK

SEQ ID NO: 158 - protein, 567 aa, Bifidobacterium infantis
MSGTQAHHAGGTRARIGRAATGVVSSTAQFGSDRTDIADSMGHTAAEMAGRAGMHGMSSTMHGVGWTAGRARRIMNRGKRALRSGRGMRKTAGKPKALSEAKPSDEIGKFAAKGKASKRIGKHIGAGLGKAGRSVKRMGSTGMGWMDEAGARLTAADDDFASKLGSTTRDLSFKAARAGVKGVNSSAKFIWRHRRSPAKAVRGAKATGQAAVRAARAAANFVRMAASRVIAGAASISLPIMPVIAAMLAVLGVLLAVMGAFLGSSASESTVSGVPAEYEADVIRAGSICQVVTPSIIAAQIDQESNWNPKAGSSAGAQGIAQFMPSTWASAGKDGDGDGKADIWNPHDAIWSQGNYMCGLASQVETAKKSGKLTGDTLELTLAAYNAGLGSVLRYGMVPPFEETINYVRRIKELAATKYTATGTAEGGTVGSLEPKLTVSGGIVSTAGITPDTRYPWGQCTWWAATRRADIGKPIPGWGNAATWAGSAASAGYTVDGSPSAGSVIVFQPGVLGASAGYGHVAMVEEVRGDGSILISESNALGLGVVSTREISASQLAAAGSGVRYIH

SEQ ID NO: 159 - protein, 179 aa, Bifidobacterium infantis
MEPKRNRIIAATGFAVALLILGGNVAVICAGNGTTEDTQSTVARPRTKTEPGQKTAGDEETEPATPTEDDPCADLAPKALGVYMGDERGQLEGEYFTPDAAGLDIPASSIAPQPLPETEFTGFPVSTGRRVATCAVSTGLEASWVLDYTLTDDGWRCAAVKGPLEGGYRVHEGKPEEQK

SEQ ID NO: 160 - protein, 460 aa, Bifidobacterium infantis
MTRIDRKTGEPILSPKLTVDQLYAMANEPGWRPWMRLIAEHPHAWPELAEWWHTAQEQGFDTAGAAPLPPASMRGRRRVAIPSAPLPPEDEPGQEPVSAPAEQSPPDDSAEKALKDADDDFAALERIADLESDTADIPPIPEAESSGPAVTYSADPDDLKVRRVFPVGKALVAIVMAASLIAVSWMGLQIKNRRAAAMRQEAHETAISACDSAEATRKTVQSDLDRTTAKASRLLKDTSRGQVAEPKTLDALNRLLDAKTSTIKGSCAPDAVTSDVDRTTAALRRTTKELKNRLTDLKTATKAVTDSKLDKTVDDANALYKQTDGKVADDKTRASLLDAIKKRDADAIAKAVKEVNESKMAKEKADAEVKAKAEQEAAAAAAQQAQASQSQSVPQRQTPSYSGGSQSQSQGSSGSGSETVRRPSSGGSSSSANTGGASPGWSVPAPSDEGTGLPGSDPGL

SEQ ID NO: 161 - protein, 106 aa, Bifidobacterium infantis
MTRTTVSGIRPDKTGTGEWIDINGHVGRLMSTLMADGRPFSIDADPAGMTIVTPLPPPAAGMETKASDGNNAKIETGSDEGRITSRKTLYERFKTRLNENLDERKQ

SEQ ID NO: 162 - protein, 101 aa, Bifidobacterium infantis
MTTEANDRPEPVIWFEGTLIRDPQPHGGQDDWLLETLADADGPKITIHASGEDHSANIRDNAHRGSRLIVKGTAGDEGSGVDIESTSLAFDPSHDEPDGEQ

SEQ ID NO: 163 - protein, 672 aa, Bifidobacterium infantis
MNQQEQATKSAAIFQDTINGTNDPTPWPVTMWASSGDTIWTAGTARTAGEDSVGMIYGPGDTIVHRNTIAGDTTRATESFAIRPADGQSPMDAMLAGIEQWNHRHPDRWDTVTTPGQYRMTDPTTGRPLPMGWSASLAAAASAAGRLDPDLLQASLMQNAVEHEPRPCVFFLEDNGYDLMVFSWHRNQQGLFDAMSFKHLQYDDLSMQVTTINHSEDMSESFPAKTMSDGELLTQSRIYRDEYQHWREQDGGPVAHGMTGRVMRSGLLKPGLEQKPLLNLNDGRRAPDWDEFTDQAAMAILQGRPISPTPALPQQEQPTQATDPAAATQTPARTAATTKQVWPNAWVANRLAHTYILRAKDGRDWPKMIVGLPRGTAIDEQDLTGWATDMFMSGKNQKQKNEGRAVNLRFKPDTPVELFTGRGTERRTMQVDPQTLVQAIIDAQKRNRDAEETLDTASVELASKTVEESWPQISRMQGRFTEYQRAGTYKPAVAMKWARRLVDRTAETDEERWTNRQRRQAAGLLIQELAGTQEPTQDRDKTASRPTVEQDAEATDTPRKTEPTTSTEQGRDHDEAGSPALAAVTETKAESPETTRHDKPERRHTDLKSTLDRFRTRLRENLDGLNTAMPAPGLDQLDEPTPERGRDLDRSEPKRPAPEREQPASKCHGLGL

SEQ ID NO: 164 - protein, 357 aa, Bifidobacterium infantis
MPIEEQAEEKIQNAVQTIITGGAKLMLRIPKGVAMAMLRSGMKLTKTGVYAAGEAVKNKIDSGEMSEKRLQRKKDGDLHELQLDDSTMREVQHSLKTAGIDYHLERADQGQFILHFAGKDEDHVRHAVQRAFKGMGLDVTDDDFTVEQTEQQERTTEQTRNEPETPARETPVEPAAKPLPPQRIAWDFVDPEVMEMAANSLAARHPELSWDKLMGDTTWNEQTGRDFADRIIGKAAADPSLRDELDAILRDDYGQGTQTQEQNRAQTPEPSRETPATEPENQPEPTPAEARAQAEQQQTQARADNPRPAGKEQKPKPIRSKKALLERFKTRLNENLAEQKNHMPPTQNRDRTPRKGR

SEQ ID NO: 165 - protein, 542 aa, Bifidobacterium infantis
MAVVKLGKPVKSNLGGPNGAMAYIIDPAKTDGGRLVSSNYERTGTDYDALADPMLEDNENSPKGIRKNSRLAYHIKLSFSPDDPVTPEKVHELGVEFAHRITSDEYKFVVATHTDRHHLHDHIMVCAASRYGKHLKAELPKDIIDQWRAVSDEICRREGLSVVFNPVVEKQTRKMRDGTTAEGDDGTSPARDPKYVDEAPVRSANEPEATARKASGGEPLERRYGMSMEEIYASAKGLGVKDRLRMLIDLTSSMAENFEDWKDILDIRGVDVTVRGQHLTYILKDTGFKIRDTKLGQAYDMTNIMAGLQSTPVIPITFNRRLVAKQTRKTITVWLPSTHRRKKITFDAKRLVDDGGSTLRAFLPRDRDQIILDPSNRYAGKTPTTGLYQWFGEPTSRLEPLTSPERLPLRYGVSPAQQRYYQAQARRLDRLAGEAKALNAAIRWTRLADGDSAKGLRLLRAKVRESHDELQAAVIALHDAIQQGDPDLVAETRGEMERREALCDRYEDELSAIEREIHTTRDREQTETEQREQQQHKRGRSI

SEQ ID NO: 166 - protein, 126 aa, Bifidobacterium infantis
MSWAAGRDRNICKRITFTQDEWEQVRNLYEELTRYAPEHRSFSSYARKMLSERRIHVTEIRPLTDPEPLAKEIGRIGVNVNQIAHWANANEHITAEQVAELRASFARIERLLGDLFADKREARKDV

SEQ ID NO: 167 - protein, 82 aa, Bifidobacterium infantis
MTIDYSIKVTQCEPMPGYKLKVTCSDGATGIFDMSRYVERGMFKPLKDTQTFDRVRLTFGVPSWPGDIDIAAERVRSDMQII

SEQ ID NO: 168 - protein, 95 aa, Bifidobacterium infantis
MEDNESKEADMPVISMFFGIIITMNADDHVPPHIHARYQGHEASFTFDGNLFKGDFPRKQRKLVEAWVLLHAEELEADWDLAFNLEHPFRIDPLR

SEQ ID NO: 169 - protein, 91 aa, Bifidobacterium infantis
MEAICPAGIDLDRYATLANGNVGYDEADAHWDDAAWWDEQERAMWHGGHGSLTGNGVRHRCRAHPEDFLLAGRLTTRQLTRSETGGFDAFS

SEQ ID NO: 170 - protein, 55 aa, Bifidobacterium infantis
MKGDADMKEKGRAMEWFRTKHDELAGTTRTNFAAHGLEPGREAYHLGSIYYATAE

SEQ ID NO: 171 - protein, 97 aa, Bifidobacterium infantis
MQMEPMELQHVVFETDEGRVDAMWSDVRLHTHDIPDGWHCYAVRGDDGGWPPCSIEKSAWVNHAGDIVTPDDLDPLLERNDWMLVIRDWWFTDEPFE

SEQ ID NO: 172 - protein, 143 aa, Bifidobacterium infantis
MASDYVDDFLRDERESFKRKETAAKKFDNRLDNFRKSAQALRDAARQFEEAFPGMSRTMIADKLAMTGTEKNIAFDAKGELVAAPAGRKTPPAEPEPETVAESNPETTGSDVQAADDETAPQEPAAEPDTSDDGRPAWASGNY

SEQ ID NO: 173 - protein, 783 aa, Bifidobacterium infantis
MRLIIAEKHSVGQAIAQAVGGHAEKHDGYIQVGADLVTWAQGHLVDLAAPDEYKNHDWGKWSLDTLPIDPTPDWQWKVSRDKGADRQYKVVAGLMRRDDIDMLVDACDPDREGEAIFRRIVAHVGVSKPMRRLWVASLEEDAIRDALTSMKDETEYQGLADSAMIRAKADWLIGMNASRAYSLVYNARFTVGRVQTPTLAMIVDRDRDITGHVARPYWKVVAPMGGWKLAGERLDKREDAETLLRIVNSDDFTFKILKADRKQQHDAPPRLYDLTGLQKDMSRLHGLTAARTLAALQSLYEKRLATYPRTDSQYITHDDLDTLRGLTEGDRLVTGFIEPSAKPERPRLELTVDDTKVAGHTAILPTMQAGKATLDELGDDERLVLIRVARRMWEAVGDDYVHDVTNVVADINPAWGERHPTDGTTLDESQTRFASRSDQPVSLGWHAIEHDGPQEEHDNDEAAGNIIPANLVVGVSIAPVPQCGATLSEGKTKPPKPFSEATLLAAMEHASRWVEDKELKAALDDDESHSGGIGTPATRADVIEKLIHTGYVERKGKQLRSTEQGRSLIDVVSPKLKDVALTAGMERRLSEVEHNHADPAQVETEFRDLAVRIPADARSAVRQDHVQTRTRDTESFGPCPRCGKPVIKTGKVFQCSTNRREKHRRHMEDHGGMRLARLDDRGRQNRHRRDHAQAARRSEGQREGLHLEEGRRIRRHAHHRQRQGHRIRLQRQQGKEKEMTETQQTMTEFTYATGGGRHRRRPVQPVRGPVLAQDQRRREQHAR

SEQ ID NO: 174 - protein, 85 aa, Bifidobacterium infantis
MVLFWLRTSVAASNMRGDVQEPEHFAGRDPARLVFADVEGLRHQESMDMAQKQYTAQTNSVLIQQQMLEAEGVAVGYTYLPEETE

SEQ ID NO: 175 - protein, 142 aa, Bifidobacterium infantis
MFGLYMLAIYLGVAVIVAGFVALLVIVALAVGLVTHPIRTLALVFHKLAALAAGLALILALVVWFWTDHAKPDFVPCFWGSIGVIVASVLVRALAEWILERPTRAERRAMRRVAAPTFDSQDQGGRGPSHVLSDPQQGGSAG

SEQ ID NO: 176 - protein, 240 aa, Bifidobacterium infantis
MAEAQEQEEPLSEAKAGKILDAVYEKILNGIPLVSRSVDEIADDYLSHHDTPEAAARSLARWQVAKCGTSGFLSGLGGIVTLPATIPANIGSVMYVQMRMIAAIAKMGGYDVNSDQVQTLVYMCLTGTTIADIVKETGIQIGAKSLTAAIKKIPGAVLTKINQKLGFRFLTKFGEKGVINLGKLVPIAGGLIGGGVDIASTIVISRNAIRMFIEDETLDMSEPTEEEINAAEDITVEATD

SEQ ID NO: 177 - protein, 202 aa, Bifidobacterium infantis
MPFLFSLLPRRRLEYGSAKYVAMPGPSRRRVWRANSVPLSCVTPPRAPAGSGENITSCAPTLSPAVLPGTIPAMRNLVFLSTSVCGLHPAPITPSASQWPKPLRSPASGGRSEMGTRPGIGKRDVLPPPRLRRLPWPRGRRRARRSRRWRSAQIQRQTVSGHTRMAESSGNRTRGPPLTGGGDQPRPSHSATRAHRRSHDRR

SEQ ID NO: 178 - protein, 48 aa, Bifidobacterium infantis
MEQIAELLKAIGEFLSGLGAALAPIAAVAVALIAKSKPRKPLNRRRKR

SEQ ID NO: 179 - protein, 49 aa, Bifidobacterium infantis
MNGKIGIIALMFGVVSLALAIASQSVPAGVFGMCSGVLGYLAGRASNGD

SEQ ID NO: 180 - protein, 368 aa, Bifidobacterium infantis
MTWTQIDDGLNFSPQTMPGTVSNAALGLWVRLCVHTAYQLRFPAFDGAFDLTVVRSLKGNARQVTELEAAGMLEPALAAGRWMVVEADTLMKFGGTSGSELKEKRAKAGHAGGVASGESRRSKREANASKQNEASASSKPRSKTEANHEAKDEANGEAKPKQTSEAKRSNCFEANEATGPNLTIPSLTSPVAPSAPNAEPSQAVAESGHARPVSSLAEAEALAEADPFAFAWDRYPSHTGSRDQAQNLWQAVTSGSDPTMPQAEPSQLLGAVIRYAQTVRQDGDRFVPSMRKWLENRQYTQWLQSVPKRTEWGGVTRQWLQTHAISQVPEGSWTDSVEQTFWAHVKTGEEPETVAQRLVTEINERHQA

SEQ ID NO: 181 - protein, 227 aa, Bifidobacterium infantis
MVNAAEYLIGFFEAGTTDEADRQGYRFPDVVQALSEVESAIDSWESMGVDVHLMRSCLERWKKSALNTFMDIDELRWDMSMFTHAKTQEKLTDGDLMGLQSVAEKLSASTVSYSEDARQKMRNMIEEAVKCVRADDSLPSDLLAYLSRLIREAREALDEYELTGDFKLSVAFDRLCNALRVAETKTKKHPVWEKFNEQFMVPLIAQVGVNAAVYGLTVAQVLPAIGS

SEQ ID NO: 182 - protein, 194 aa, Bifidobacterium infantis
MSHEDINEASGDSRIQSGADSGADGQNTEIRGSENHDISRIADQGVEHDRRFDSEGDGSPVVSTSLTHMEVTSGPLPSSKELAGYEQTLPGAADRILRMAEDSLHSEIDSQKRIVEIYADDRKAENWVYKFTTVVFSLVPAAAFICSVVFFALGMNPAAFISFAGSVGFVIPRIIEAFKSGKASKNEDKPKSEN

SEQ ID NO: 183 - protein, 58 aa, Bifidobacterium infantis
MRSSQQQAISIRIIVQKERQIGSLQDEVDGLKKILRDQSWQDHNHDICQETGNFFWLC

SEQ ID NO: 184 - protein, 110 aa, Bifidobacterium infantis
MTKQVRILKNDSDAGGAKPIAGFLSGRLFAAHMYAAAIRHRKKTRDSENRCLSGTGIALNSKNFPTLQRKGYRSENEPAVFVPCITFRHVVEDENAAFDVPHHNGAAPDL

SEQ ID NO: 185 - protein, 187 aa, Bifidobacterium infantis
MSATVAWGDESIRHKGLARPMYLMCACIIDDVPAANQSMRTILPEHSKKLHWRDLTNRRKRQSIDFIQQIGPLSIVIAAEESSVHKDERARRKCLERLLPVLESYEISTLVLESRTLPQDQRDLEHLAQMRTRHLVRTIRMDHIRGSAEPCLWIPDQVLGAYGDTKTGKLDFSEFLRENVLDEIIAC

SEQ ID NO: 186 - protein, 84 aa, Bifidobacterium infantis
MLFAGIHPLCRVHDIPLVDISHKTGIGCVNRNGIALILVARLPHKRRRTRLEGCTPEEFRSMSLAVSDCIPLTDVQQTGCGSVP

SEQ ID NO: 187 - protein, 173 aa, Bifidobacterium infantis
MLLPCTVRYRFRLRDSAVSLVSATKKPRILQGLTDCVSLIGPAFVDGASSINAELVRQRRRANNSCGYKRRSIESSHRVASFRYFLRPFDLNWNVNIGLAVLPIQQGFTLWTPRRRDERPRRGEERRRRPGSRTGPSLRGLGGEGDDSGQQHHDDGGREEQVIAEAVLEGVHQ

SEQ ID NO: 188 - protein, 76 aa, Bifidobacterium infantis
MLFLIRGNDKWPKCFAEELRLRQFIETLKLLGKIILIMMESFRVEICNDSLRMLTLITSLSFTKIESNGIYFFPLI

SEQ ID NO: 189 - protein, 141 aa, Bifidobacterium infantis
MVRPASMTVSTALTMAANPHSAATTRMSSSHTRTDARKPSADGIGTIGSRPAKADRVLHAALPVAGIRVAEPGPEPVARAEQGEQTGPGGRAVGVAVAHADGVVEHHDPGRHARPLEHLRQTPAHAFRGLARQRRHLFVFN

SEQ ID NO: 190 - protein, 318 aa, Bifidobacterium infantis
MIAGFSITSLLLILLAGIGAGFVGYAVGASSLVSYPALLAFGIPPVLANASNTVGVVGTGIGGVMGARKELKGQAVRSITYVVIGAFGGVAGAFLLLKLDPSVFEFAAPVLILLSSLIIAINPRGRMQAKQAAADATAQLKHIEAADAAKRARANSAASDDPAGGAGADSTAVSSADSGAAASVAAPHRPPEQLVQPMNQDSWWVWLGVVAVAIYSGYFGAGAGTCALAVLDAAKIGPFHKINALKTLIGTGANISASVVFIIQGAVDWPAAIMLCIGCFIGGYIAPPITRKIPANIMRAAAVIAGIVLTIDLGLKTY

SEQ ID NO: 191 - protein, 175 aa, Bifidobacterium infantis
MRPFSKKFCVGLVIAGIALNVLYWLAAETLHLCTGKFLLPSDFIFDYWKLPSMMIGFVMFLLFEKHHFSSKVVNWIAGSAFGVYLIHYHPVCHELWSTYLPVKNLIRLSHPILNGMACIAGIFLCCLLLDMARHVIFALTIDKHKGAFFDKLYAYAEKRKIMSILKATNTAITNL

SEQ ID NO: 192 - protein, 332 aa, Bifidobacterium infantis
MYGLEPQSTTDQWDYEQGLTEIIDALVKGADLIDLDYFVHVLVPYASGLFMRGLDYGERQENIDVIRYLRDKNTILTDNTVFSRIIHLQRILAIVLGAEWTLYEASPRGQFIQSDMGFCPASDHGKRCWIVPLDPKLVILIKPKKKHCIANYREINDMQGNRSASWVVCFRRGKLNDEQMLQVNENILEHSFRICIGDGLRSFKDYSYEPLSEEKNNYLISIGAELENSLEVSERVEHEFEWYTVSRIAAERLSPDKAKEIQWNRPYRGSYPGRWRPFMPFLPENLKLSPSGLCIKDNRLILSLRETVPFSSLKCSNTFESNMAQIQELVNR

SEQ ID NO: 193 - protein, 280 aa, Bifidobacterium infantis
MSVSSDAFHRLGRSRFQAYADFARNSGIFPATTEVDGIAMELYRWNAEASAVLMRYIPWIEVLVRNAIDEQLRLWLSRQTPQPYDDWIDVADTHPMDRIRALINTAEKDYLSEARRTALSKKRFWRSDQSHPRHGDEIDRDDVFAQLTFGTWDGMLSRAANDPELARILMGAFPNIENAWESETRRMPNSSLPGNESDSREDRLRRELISRLKGIRVVRNRIGHDENLLRVNFPKIRHDMYFVLNSLGADYPRWAFPDKAELLKRLNPVQVLEQLERKGK

SEQ ID NO: 194 - protein, 65 aa, Bifidobacterium infantis
METPRWMGLFGDAVMTQANKTPFGFQSPASFKFLVRFELVLDVHKAKTGHIAIIYYICLKLVDCF

SEQ ID NO: 195 - protein, 287 aa, Bifidobacterium infantis
MAHNNLEQLNFLIRSLDSEFSDIFLHLDAKSKINPDEIVRPVSSQLYFCDRINVYWAEYSQVQCELNLLRLATRIGKYNYYHLISGMDFPLKNQKEIHTMLRNRNDIFIHFTTHKNVELTIPFVRYYHLFQKQLCVANRDHTFSIYKVFEKISILIQRLIHLNRISTDIVIKKGANWFSIPDDFARYVLDHQDCIKKLFNNTRSPDEFFIQTLAFNNPDYKKRIYRFKEDDSSESCLRHIDWMRGSPYTFTIKDYDELVNCGMIFARKFDLNKDRQIILKLMSHLSG

SEQ ID NO: 196 - protein, 331 aa, Bifidobacterium infantis
MYSTFWKVTGFKSFIKNALWATVYKRIKQQAAAYEAFEHKYFRLSQDTYTSNKELTATNSKYQKFITGSDQVWNIRCMDADDAYYLNFVSDFNRRYAYAVSFGANNPFADSSLKDHYLNLVNKFNKISVREQNAKKWVSTATGREVSLCVDPTMLLSQGEWEKTVQLGSAPIIQGDYIFYYCFSISQEIASFLHQVSKKTGMPVYFFEPKEWALRCCWKNKIRLVKKYGPEAFLNYMKYAKMVFTTSFHGTAFSTIFHKNFWYIDSGHNDLSKDDRAVSFLTQLKLTDRYKTISSLLQTDLSLIPDYTQPDQALAKLREEAFEYIEGIVND

SEQ ID NO: 197 - protein, 490 aa, Bifidobacterium infantis
MTSLKKNLVYNMAYQILVILLPLITAPYVSRVLGAAGLGTYSYVYSISYYFGLVGMLGITNHGSRSIALYRTDKVQTAQTFWNIYAIQFISTGIAVFSYTIFAFVLFNGNKIVAYINILFVISYLLDINWLFFGLEQFKITVIRNTIIKVATACSIFIFVKDRNDLWLYTLIMALGASLSQIYLWMNVHKFVSFCKPEWSEVKKNVKPVIILFIPAIAYSIYKVLDKIMLGAMSDMTQVGLFDNAERIVNIPVSLITAFGTVMLPRISNLIAAGDSNQIKKYNAISFRYFTMLVCGAAFGLAGISNILAPVYFGDEFKGSSPIIFGLSFSLIFMIWANIIRTQYLIPNKKDMPYVISTLFGAAINISVNLLLIPHLQAVGTMIGTILAELSVFCVQYLFTRKDFPTLQYLKSGIMFFPIGALMGIIVWIVGQILGPTIITLIIQIILGAFIYGIGSLIYLIAIKDDIFISMFKKTFHINANSRLPKHRSV

SEQ ID NO: 198 - protein, 399 aa, Bifidobacterium infantis
MKLSKNLQILFIALAIIPFFQSTIGYKYPILGLGVQYSRYFFFGIIIILILINTHAFSFITRNTKIYLTISLIFIFFISAVVNNTNIVSVFQLSLYLLYPFLLFSVWADSSTDTLNICKGITYGLNTLVLINLSIMLFFPQGLYQTISSNTISYYYLFGAKNQMVAPIMTCLFFNMETAYRSYNKIITKTSLFMCFICAFELIIGGSGTGLIVLAAFIILCLLELKHQKISTNLSLIVLLASFLAIVIFRIQNIFSFLIVDILHKSLTLSDRTYIWDAAIESILSHPILGTGITDSLSGNVHLKLSYLVKDIFAHDLYLDYLLMGGIPALCIFVCILISVKKSYDSFLNNKNTLIWWGIVVYLFASIVEIYTTNFCLFLMFAYINICDYSTRHRYIGQN

SEQ ID NO: 199 - protein, 331 aa, Bifidobacterium infantis
MSLVTIILPAYNAEKTIARAIQSARCQEYSDLEIIVIDDGSSDSTLNICNALADKDNRLKIWHINNSGPSAARNYGLSQAKGEFICFMDADDEMSPTMISQLVSNMQDDTDLVACGYKVRSNNGDYAFEQKLDDKSYPNNRLYEFIENLQEAKAFNPLWNKIFRRSIINDNNIKMDTTVDMGEDYLFVVDYCEKSEKDFKALSSSLYIYELSNNGLQVSANKNNNLKRRLSQLDKLENLFKRRNYPMNAIYKEKLRIIYTSLLESNDLHNDLIEVLKNGYLDDLIHSNINLGKKYTVFLEILKSRHIGVAYLSMNIFRAVKKIQGKSFNWG

SEQ ID NO: 200 - protein, 221 aa, Bifidobacterium infantis
MSFSPDITNFDKLIVLVNTGIKFIRGLWMRLFLREAHGLLLIGRNVTVSHAHNVRCGKNVKFEDYAEIQGLCSDGLNFGDNVTIGRATMIRPSSYYGGDCGQGIAIGDNSSIGPHGYIGCSGPIRIGNNVMIGPKCSLFAENHVFSNSDESIKSQGVQQRGITIGNDCWIGSNVIILDGVSIGSHVVIGAGTLITKDVPDYSIVLDRRNRILRQRIKKIED

SEQ ID NO: 201 - protein, 143 aa, Bifidobacterium infantis
MGGGPLEDDFKAWADKKPNVYFLGYTPHEQCMAIAKAGEFVVFPSIWYEGCSMVEIESESLGLPLIATDLGFSAEAVNDGVNGVKVPLGDMHGWITAIRGLWKEPELCKKMGMNSRRDYEDKYTPENNYQQLINIYQSTLERG

SEQ ID NO: 202 - protein, 47 aa, Bifidobacterium infantis
MLRRIWPLNVYQGYLSGMQFFMVALVLRKAFVYFNAYGMRLTTFRWW

SEQ ID NO: 203 - protein, 397 aa, Bifidobacterium infantis
MSGVSAVQHVFVVGSKGIPGSYGGYETFVDRLTEYHEGEPGLRYHVACKSLSESGEFSYHNARCFRVRVPQIGPAQAIWYDVAALADCVAYIRRNRIPHPIVYVLACRIGPFTAHFASAVHRLGGKLYVNPDGHEWMRAKWSAPVRRYWKVSERMMTKHADLMVCDSENMEKYIREEYARYRPQTTFIAYGAESRRSRLADDDPKLLDWFRSKGLEPKSYYLVVGRFVPENNYETMIREFMASNSKRDFALVTNVSDKFLEELKEKTHFDRDPRIKFVGTVYDKELLMKIREEAYAYFHGHEVGGTNPSLLEALASTDLNLLLGVGFNREVAQDAALYWSKEPGDLAALIDHADAMSPDEFHALGARAKQRIADAYSWQHIADQYKNLFLGREQKEG

SEQ ID NO: 204 - protein, 86 aa, Bifidobacterium infantis
MIHNYRSNNMEKPGIIDSRFSGIRELCFQCLPCIMSVTSATSGALADGEQGDARGGKQRNGHTAAIADSTVIIFFFITSNLHNRTG

SEQ ID NO: 205 - protein, 685 aa, Bifidobacterium infantis
MMIWLSFFVAILATVFILYAPGFLLMKAARQTSINALAFAPPLTLAIYAIVEIVLSKAGVPGNWYTIFLPFLIVAILLFIFSICMRHKEKRSIRCALTNLSDTFPYWMMVVLYVAVGSVVSLIVFIRHMHGPNSYTQLYDNAWHMGIIQKFLASGDFSTLNAGDIVATSGSTFYPTGWHSLVALVASMTGFSVPVCINASIFVILSIVYPISMFVLVGKLSAYNNNVIVAGAFTALMFAAFPWRFLTFGPLYSNLLSFSIIPLVITVALNMVEAKRGVKERIFLVSVFVLSTIGVAVTQPNAVFTMGLLVAPYIFLQIPGYLEYAGITKHRNIAVAGACVALMMAIAGVWSLIYNAKFMQRTVTWQWPSYEKKIQAFIDIAFVGFRNAEPQILLGVLVLIGMVYTVFHKRLLWITCGYIIMCGFYAVSSSTEGFLKNVLTGFWYHDQYRLGASAVFYGAVLAAMGLWNLMIVIVRLVPENLIGASNSRNKCILSTFCVLLIVLVNYFPSFYLSGRGDYVTAFGAITRDISYWNSPSEPKSYTATEAAFVEKVKKVVPKEAIILNQPYDGSAYAWGQNGLNVYYKAWEGNWMGAPSDDNSLISESLDKIASNREVKEAVRRVGAKYLLVLDKSDFSKDKNDVSMMKSIYASYPERKWSGIDEVSDATEGFKVVLSQGAMRLYRITV

SEQ ID NO: 206 - protein, 392 aa, Bifidobacterium infantis
MFKLRDMKWFNAGPRKSRARRHYLVSTAGQPNWGDEFITRAWIRFLAKSDPDSEIWLDCPNPSHSSLLLKDEHPHLHVVNTLWQLVWNTSDLLDDSEAAAQKIRGWIRDGGTPREDFGIDLLRSMDTVHLLGGGYINQLWKANVLLLVALAQLKELNPKVLLYGTGLGLAPLKGIDLFLARTSLTAFDHMSVRDSRSADIAGTERGFDDAFLEIANTGSDWLEQNASARAFVCLQQDVVGRNPKAVKAAVDSLLLSGVEKSEPIFLVEAIPPEDSWSLDLFKDQWPGEVWLLPFSHLWNYGFPNAADTVWISSRFHMHLLGACAGARGIAMGFGNEYYDIKHGSLMDLGTGWANLDVSVDHPAPVSATASSKFPAESLRIARKKKREANQLY

SEQ ID NO: 207 - protein, 562 aa, Bifidobacterium infantis
MRHFFHIDEEGARLIEKLSERQRRLTLGAVDYNALIQPGRTLTYKLYMGQKWQYPRLTDTSTLNGVLTEQAGLEPFAVFQATSSVKEKSYFSPHYFAGDEQLVDRNIRCLLLTPDDATDDDVEQNRPVDRHARLYRKVESEAPAFFDMLREAKRVQRERIGKTAIAPFEPATITRVNESGYSHHPWVEIVPGKAAPDAPKAVIIAMHWLQSGGAERWAMETVTLARQAGLLPIIITDSDGHQPWITRHEFDDAVLLPLTLPLQDRVGDATLLRALFEQFNIVGVLIHHCQWMYDNVWWVKRYFPETHVVDSLHIVEYIFHGGYPSESVARDKWIDIHHVISPQLEHWMEDVHHIDPKKVVDAPLVGLTADSKTPTFKSRDISKPLTISFVGRNVRQKRPETFILAVRELNKKFPGKLRFIMQGNGDMDEFVDRLIERYGLGTVIQRCSMSTPVSQTYAQTDILLVSSMNEGITLTTIEAVSAGIPVISSNVGSQDTLVPPQGLCRRGSAQFVHDATAIVGRVLESEENRKKLWEAEKDRLEKFSELQSANEYFRNMLTEWSH

SEQ ID NO: 208 - protein, 312 aa, Bifidobacterium infantis
MQNTKIAAVVVTFNRLEKLKKVLSSLEAQTRLPDQLVIVNNAATDGTDSFLKEYAANFKYSDSVQLDIVTLEKNEGGAGGFSAGMRRAYEIGCDYAWIFDDDGYPEPDALDKLFKGYGDAVAELGPDVPFACSLVKFIDGTISEMNNPIPTWDWGRLKAKGLDIVLVSRCSFVSVLIPRWVMEAFGLPYKEYFIWFDDAEYTLRITRACPGVQVLDSVVLHDMGVNRGVNFGMINEKNVWKFLYGARNEASYHLHHEGLYPYLRFCAMVRNNMRQGNVDKKLQRQVYGKLLEARSFNPQIDFPQTPISKSAH

SEQ ID NO: 209 - protein, 87 aa, Bifidobacterium infantis
MDRSRGYSLTQHPDPELYCLGHLVEEFDFHAGAKGIDEKGHGEVEDVTRRMVRLEVPAWREPVTATLIPYFAWANRGENEMTVWLRG

SEQ ID NO: 210 - protein, 239 aa, Bifidobacterium infantis
MGESVRDVLFLIVLFAANVIQAITGFAGTVLAMPFSMLLLGTNTAKVVLNITTLLACLWLGVQHRAHIRWRILSEMVGLMAIGMAVGVALYAVLPLAPLQKAYGVFIIVIALKNLIWPSHGEPPYWLLAIIVLLAGVIHGMFISGGALLVIYAAVRLKDKDEFRATMACVWVALNSVLAVQQGVSGVMTPHALVLSAVSIPPLIVAIIIGNRLQQHVSQQAFLKLTYVLLVISGASIVL

SEQ ID NO: 211 - protein, 392 aa, Bifidobacterium infantis
MTVRLKELVEQAADKDMVLVAGKGGLERPVRWVHMVENEEIAGFLEGQEIAFTTGIGSETQEDLYPLVKSAYASGATGVVVNIGPFIHQISPDTIRFCDEHDFPLFKVPWSVHMAQIMHSFSLAITMSEKHSMELAAALKNAIFHPDREEMYLEYLEQSGFGKDWNYCVTVFSACADAQQGGHDAERIAKYSREAESLITRNQWRVAVAHIEERLVLVFARYTAEQVELMVREIIAAIRNRGVLLDRTYIGVGKVTKSARCIGKSYNQALKLERLQHLRGRVGEVALYDNSGIDKLLLAVSDRAILEDYYQDSIGALVEYDRVNGTDLTDTLQAYFRFSGSVKETAASMFVHRNTVSYKLNKIEDILGVSLSDFRTREFLSVGLQVREILDC

SEQ ID NO: 212 - protein, 453 aa, Bifidobacterium infantis
MSESLATKELTGEEVAELHREYVMQSWHKQGEPVMPVKYAKGIYVYDYGGNKYADMSSLLVCSNLGHELPEIVDAIKAQADKMCFMAPAYASEPKSKLAKMIVDLAGNDFYQRVFFTNGGADSNENAIKMARMVTGRQKIFSCYRSYHGSTLGASFASGDWRRFAVEAGGAAPSFVHFMNPNMYEDGFNRGEDDEKVTALYLKRLEDQLIYENPDDVAAILMESIVGANGVILPPKGYMEGVRALCDKYGILMICDEVMAGWCRTGKMFAWQNFDIKPDIFTFAKGVTCGYVPLGGVVVSKRISDYFTDHVLQCGLTYSGHTLACAAGVAAVNYYVEHDIAGHVKEMEGILKPFLEDMQAKHKCVGESRCVGLFAAMTIVKNKETRELMSPYHTANPVMGKIMGALKDKGFLTFGRETNVNICPPLTITADELKAELPKVDEVLTWVDENFCD

SEQ ID NO: 213 - protein, 376 aa, Bifidobacterium infantis
MLWDYQQPVAIRFGNGRVREIKDVAAEMGLTEGGLLVSEKLFATNGTAEKIVKDSEGTISEIFSDFSPNPDVTEVDKAAALIREKHLKFVVAMGGGSAMDLAKSAASIAFTNDSIADYHGTGKAMPQEHLPIIAVPTTAGTGSEVTCVSVLTNRALGKKAPIVSDGFFPSVAIIDPELTYSVPPHVTASTGMDVLSQAIEGYWSKGHQPICDACAIHAAPLVFKYLPIAVAEPDNAEARQKMCEASVIAGLAFTLPKTTSSHACSFPLTNIYGIPHGEACGLTLDWFARVNADAQHGRVQEFARAIGFKNVDAMADAIQELKVKVGLRTGLKDLNLNAEQIADLVRISRHPNLYNNPVEIADDMLQDMYEHLAATD

SEQ ID NO: 214 - protein, 563 aa, Bifidobacterium infantis
MATLYTGGTIRTMSAPDATAEALLTDDRGHIAFVGSLAEARDRAESMIAVGGRGVQERDLAGACLMPGFIDSHSHFAGMGQRLTDADLAGCADFDEIAARLKAFLAAHPLPAGGVLHGFNYDHNELAEGVHPDRRVLDAALGDVPTVIAHVSGHVIICNTALLKLVGLNRDAEAPEGGVYGRDENDDLTGYFAETPAIMPILMHSAVMPRQTLGDLAEAIQNDYASHGITTCQDGATAPGYAEQFVKLAEAHQLKIDVTCYPMMGQDLDAVFESVGSYASGGTSEGIQYSNHLRFGGVKLFVDGSPQARGAWLSEPYMPLPTSVHSNEPAGYHGAGIVSDEAMRAILDHALERGWKVMAHCNGDQASEQFLTQYTAAYHASSRHNKIELRPVMVHCQLTRHDQYARMAAVHMIPSIFVSHCWFWGDAHIKNLGFARASRISAVHDALDYNLPFTFHTDSPIVPPDLIFAAWCAMTRITKQGVELDPAQRIGAWDAFRAITRNAAYQYGEEASKGTLEAGKLADLTILDADTLAASADSTEAAARVRALNVLETIKEGTTVWTA

SEQ ID NO: 215 - protein, 545 aa, Bifidobacterium infantis
MGKTSTTKSASSAASGTTGAVVRPFGWRDKIGYMCGDFGTDFMFVFAGTWFMIFYTKVMGVPGTIVGTLFLLARVLDAVMDVTVGVVVDRSKDHPGGKFRVTMMRFTAPLVILSFLMYQTFAIDAAMWVKIVYMSITYVVWCFFYSCVNIPFGSLASTISAEADDRTSLSTMRSMGGTIGGLILGIVAPIVIYQKVNGHQVIRGGDGTHIFAIVAAVVSIAAFIGYLICYFNVTERVHPDPDQNVKEGDKRSPLAMITNAFSSRSMLGIVAAALCLLVAQLFSNQLMTYVYTDVFGSAALASISGLLGTVVMFLVVPFVKPLTKRFGRKTICTFASALGAISMLLLFVVQTRNGMVFLLGAVFMYFALMGFNLVIWAMIADVIDDIEVTRDTREEATCFSCYSFARKIGQAVAGELAGLSLDWVGYQSGATAVQSAATKTALYNVATLVPGLFFLFTFLCLMVIYPLSRDRVLYNVEVLRVKRKGLTGEEARLLTKYEGITPTGTALEGSTIEWPDFHELERARELSARTGVPIRDPQKTDDAQQ

SEQ ID NO: 216 - protein, 311 aa, Bifidobacterium infantis
MSDATTPTPNTNDAENTPVVAGTAVAGTSIVKNEANAKRKPMPAWMKSKKIQPIISLVLLIAVWWGVTSAGLINSLYLPGPKAVWDAFIEANSCRSASAGSSRIVCGEQQYFLWQHLVVSLERIGIGMALGIVFGVLVGFILAEIGWLNRIILPYINFIRALPPLGYIGLLIVWFGIGDTTKIWLLFLAAFPPIVLATVDGINGVNRDRINSALSLGASRPKAFLFVVFPSALPSIMNGIRLAVGFAWTTVVAAELNNGIPGIGALAYLSGTELNTPLTITCIFVIGIAALLLDWLILGITHLVTPWVGKE

SEQ ID NO: 217 - protein, 369 aa, Bifidobacterium infantis
MNETLFAKRETHRETIRKVGALLVSATLLTSLAGCAKMPTADELVGNTSGAAVADCPVPSAKNEDFAGTIRIAWQAIPNADLIVKDKGLLEACLPNATIQWSQFNSGGDVIQAFGSNSLDIGLAGSSPAVRAASAPLNLPVKVIWIHDIIGDAESLVAHGGDYTSIKDLAGKNIAVPFGSTSHFSLLSALRNAGMNETSVNLINLDPDKMSAAWTRGEIDAAWVWDPVLSKLKADGGAIVTSSAATAKTGSATYDMELATDSFIKANPKVMETWAAVENYAAGLISSKENDSAESISTILGNSVADVKKQFAGYTYPQAKDQSDIFHGQLPSIFKDTAEFLKTQGSLDKVSSDYSSVLYTDAIDQVAKQ

SEQ ID NO: 218 - protein, 282 aa, Bifidobacterium infantis
MAGNNVLTWGELTEKKTMDDQGVAVDIRGVNKRFITQQGDQVTALHDINLTINKHDFICVVGRSGCGKTTLLNMLAGFEKPSDGELISGGVAINGPSPKRGVVFQKPPLYPWLTVRKNVEFGMKMQGVPAGERKERADHFLDLVGLTSAADRRPYELSGGMQQRAQIARVLATEPDVILMDEPYGALDALTREKLQNELLRIWRERHSTVFFITHSVDEAIFLATRVIVMSAHPGTVKMDIPITLPRDPDDPDNMEKVRAMPEFVKLREEITQAIYVQDDQE

SEQ ID NO: 219 - protein, 65 aa, Bifidobacterium infantis
MQIADTNTYSAANFHCRLTAATENDEKPTTINRIQMKSCMIFRFASIRIAHLIPSYSICNQNAEQ

SEQ ID NO: 220 - protein, 946 aa, Bifidobacterium infantis
MSIDEENLDSSSLGAAGESAVSYVFTRFGWSVSKPNPDRGTDLEVTPGDRHFPLGVQVKTGKSFFQKREVDEDGKLIGWWYRSSNKKHRLQWTTGAPVLLVLFDDKQEVGYWTYVSESEISDAGSSWRILVPIEQVLDKFHLSAIQQILDDYYKRLPVTETSWSNNLKDIPKEDRMRYALLTPRIIAPHPNNYRDLSGIEILAVHVLMRDELDRAWFRDEDQEAPQNRLFPIEKSFLQASESDEWSWNAAAAVHKYLCRNDSSLIMGLFDKAQAQYEKVAAAILASVVCTDNDDLKKAHDWICCARSTHNTKMDEAWLDVQEARIYLSMGDYESRVEASHKAYAAYIFVKTVKSDRTAEALLASCSRLLWQTNNPLFIPDEDRQQYIEKNPANSLMLEDHINAIDNAPQWWQGEYIGSALSKQVDFEFKGGAKNQGSPVNVDMKRKLVSAAFIASCAGNLVDWQQAWRLIAITDYSAALKNRDESLMLSSLGLLRSFGRMNEMKNATIKALHICSGQSFAEDADSIDLSKVLETDLNNTLDYLCSIAAATHQETAKRNVTWCKRWIDDTQILSAKSGDFSRGQRVIRLLFASCPAAGEEAMRETALWAYSQPAVTNNVVAGPFANGVRSLPSTVWKTIAEKRNLANDVSPVQEAFAAVTDIQADAKHSHLMNGEIDYLADLDLEHKLSEDEACAVTKKLTASVSKTIAMAKKGVHARGGLQVEVALFLIGRLHPSLRNDSLLMDFLDEQTLFHDEKEPLLNALFWRNDLLLDEDRQEWVKHINPLAEVPPTKDGFFGAQEDIHPTAMKALAANVGKEKRSELIDQMLLRGEEFTNSAFDVLSLFPDPRYITFALFTVTNSVEDALLSACRFLTVCAYQGLCNSQTAEHITTLARDGSYKVQCMICSTICGQLETNAVQGNYAKKLIAIAENCPSASLRWRLSHVGE

SEQ ID NO: 221 - protein, 267 aa, Bifidobacterium infantis
MSDIQHIGGRTPDLTEENVDKLVALFPDVLTEVPDQQTGETKKAIDFDALRDKLGDVAENTRERYQFTWPGKRAAKAEARKPIAKTLRPVKERSKDWDTTQNLYIEGDNLDALKILRETYAGKIKLIYIDPPYNTGHDFIYKDNFGKTIAADKAENGDYDDDGGQLVINPESNGRFHSDWCSMIYPRLLLARDLLTSDGVIFISIDDNKESDLQNLCNEIFGERNFVAKLYVKVNPCGRNLDMFARSNIPTTRSASARRSGAACVCV

SEQ ID NO: 222 - protein, 244 aa, Bifidobacterium infantis
MGRIIPARAGQTSWTGGRRFSCSDHPRACGANRRDGGAAGDRAGSSPRVRGKPRRRNPNHRHHRIIPARAGQTTLRFHSTFRSSDHPRACGANRTATTNEDVVIGSSPRVRGKRLKETQTEHIHRIIPARAGQTTCPNCRRRACPDHPRACGANTSEALALPLTVGSSPRVRGKQVRRRGSAIPCRIIPARAGQTDSLDPGSMFQPDHPRACGANWANMGAADANAGSSPRVRGKHRGWFWHTG

SEQ ID NO: 223 - protein, 317 aa, Bifidobacterium infantis
MRGKRVLLVETVEHIRIIPARAGQTALRDREKPETPDHPRACGANRFFPSAVVCAFGSSPRVRGKQRLTGRFRATVRIIPARAGQTASECCSRCRPPDHPRACGANLLALKAVLIVSGSSPRVRGKLAREPVLAYVDRIIPARAGQTRTVQAGIVVRADHPRACGANFKLSFALTVTSGSSPRVRGKHHGLLSAHARRRIIPARAGQTRHPIWFLRRRPDHPRACGANQPKTASTRPKPGSSPRVRGKLVLQPLTQLIGRFIPARAGQTPTNTGAPSSNTDHPRACGANVRVLSSSVVQHGSSPRVRGKPGYRLADS

SEQ ID NO: 224 - protein, 178 aa, Bifidobacterium infantis
MRIIPARAGQTWRTGPRCRRWPDHPRACGANTVLIPSAIFWHGSSPRVRGKLSQRYFVAGVKRIIPARAGQTRGHAVPAMRAPDHPRACGANHLLADIADNTAGSSPRVRGKPGWGRKRKAYLRIIPARAGQTGPPPGEAGEGPDHPRACGANLVSSLTGTSLAGSSPRVRGKRAGAR

SEQ ID NO: 225 - protein, 66 aa, Bifidobacterium infantis
MNEGVISRMARVEVTKKLRAAYRVGSKKEKSAVLDRFCEIMGLSPSSARQYLMDETIGNPKVLRLD

SEQ ID NO: 226 - protein, 563 aa, Bifidobacterium infantis
MMMRDSDRPWLRRDTIFASAPGGVLISNATTGCEIAGESAYELFSRVFPLFNGQATVGEIKGAVAERNWKLIEAIAAPLEEKGFLRWIPESDYELLDNEKREKYADQIAFLAQFTDAPHEAFLAFNQAQILVVGSDEVADSLQANLIDNGAELVTSAESFAVDQFEELAPDLTVLGPTALSGIDHLRKAGVPFLGVCPAGDYLWALPVGWTEGSASWHSADSSLRRGSMGKEWAEAIEQAQAGQPQWTSATSSQAVQRLFGALLAYEVFKGITGAITPETSEKILAFNALTGATSTHPMTPIYSEVSREVHAQLAGAHPGDAGSETPASIHVSHADEYDDVWAPLVDRFTMPAFDFDDLDIDQVPVKVSLVETATGKVFAASPWTTADARIEALARAYGQSLSWHCTWASDAPRVVGIGTTRADAIRRGVEATVRREMLSGSGLTEPVQSVLGGRLGTFVSDVAEGSLEFFAHDPLAGQHVAIACCDEYRAVGAGSSQEEAQARAAIEVLGRRQVGLVDTGDAQPIGGEVALSTACIGQWHVAVVAPAGSRACADDSALEAVR

SEQ ID NO: 227 - protein, 535 aa, Bifidobacterium infantis
MIDTIQGWRHAVLPGSDTCIRCMAEQIAMTDRFFMRTGRQLIATSPRHDNPFFKGCVEAGGRLGLAHGISIDRASLQVRSAATVCRHVRSEKMTTEAGGYRARTLSSYFHVAPVERYVSPVHAVIGAEIDFDVTGLVTAKASGFLILYVGDDYYTRPWGGHCGKLSDALYLGVLEGVERLSGEADHDAQVAVQPPANTRLIDLDAFGVPRDCWKIPHPKVGAWVKASVITSGPDGAFLLGETVMIPERLAYYRAAHDHEPWVQDSSNGCAVGGSDEEAILFGMLEAIERDAFMIAWYGNLDLSPIDPASIHDEESCAYLRRMELCGVDVIFLDATVGVGVPTVIAVCAEPSGATCVGAGAHPDPERALKSALVEIASDFQVVAEHGKDRAAEIEWMLNDFSLVRAMEDHADLFALPQARQYIDHWLHPIRRHVTLDSLRRAPEAGVSVRDDLAATLHSCRDAGFEPLAVDMRSQIANRMGLACWKVVVPGLIPIDFGSYQRAVHMPRLAKRVAAVTGVPASPASFVANPVPHPFP

SEQ ID NO: 228 - protein, 510 aa, Bifidobacterium infantis
MSQWCEPGTAAVTYADLIYHRVRDGMLINDWTVDWSKEPLTHSFYPEAAAINLPLPGEGADVFRGVAGNAPEFLASLLHISYGITGVRLTINRNDHMLVYRNAEYAKWGRTTASGGGRYCVDFYLVDSGIGHDQLDAGMYHYTPLRHAWELLTAGDFTGRVARAQGYARAADRYLIMTINYWRSGFKYNDFAYQATAMDVGTVMATMAEAAGDRLASSWDMWVNEDQLAPLLGLDQTKDGIYAVQAWGNERDVNAGCEPDVAAFPGRVTSNYPGTIDFTTTIALQNDMMGFPQRPAELLPNAVSDESPNGIAGNWWSDLMRRQSSFGRFTGRGFGKEDLLGLLSRADRVSALVAPPSSVVWEYLVYVARVDDVEPGLYRYRPFSGDLELVSTEDQSEFLASTYFLKNYDGKKAAATVIPCANVYKCARRWGVRGYRLVNAVIGAACQALSVEAARRGLGSGTALGFDAEAHADHAGLDKESMTPMLMIMTGVDDPLSGQFHSNATARRLV

SEQ ID NO: 229 - protein, 864 aa, Bifidobacterium infantis
MSVLDPYCQVRIGGLDYSHASELLDPTLADHVSQVREAYARLQAHAEDISDLLYPLIGGAEASTKTGLINVRRHLYNGKYAQAWERLEELDETSRESVRDALGSFATDLQTFITGLDYVRNHFDDALASEREKLVAHVANPLFMAGIAVSSPSLVPALLRLTSHVNKGKVDKKDRKAERSLQSYVLRAATKTSPFSTLGPIAIGYTRQAEVEQSHRSVSLPSLYPVVRALHALAEDPKNLAGFEVRVSDYVRGTDGVVSVDRTQWDFKDASTRTDYAKPTESNVIINQRPLVDAVYQILGGALMTFGELNDELSRQSGIPPETTLELLADLMRLGYLHVPALSIHPHDAPRIDGIVDEVRQAHPELGCLIAQFVERSRSFSRLEDPQMRQREIGDIQELVASIYSLAGVDGDMPRSVLYEDVVAGGFTGSELAQLELSDSEVSEIFLLLDLLDDSHVKSALMEGYFDSRQRLEISATDFIDGFIDELFDSFEAYDLGGIADDDLPDDPWLRWGEAWRWVLARRRFVDHLSRYVATHPRGSHAANLRDVGAVDISEALAQAAAILQRPQHAFRHANILIQFDGEAGSVILNDAFGGIGFQISRFTHLLDEPAHEYLADVERLAKSKGVRLVELSGGALFSNLNLHEPLFSTSLVLPGEPVSSRDVPAIRLEDLVVTKRDGVLVLTDGCEDIHPVYAGYLVPAATPRRSQVVSLFAPSAQISRKLTSLVTTTPGIETIAVIPRLTLGRIVVARARAIMATDALPTDSPLEADGYLAWLRFWADNGLPERCFVKIIDEAVQAEKPSYFDIRSVISCSTLLNDVKNAEGKAYVEVAEVLPASPTATHDGRSVVNEYMLGISLMGGSDA

SEQ ID NO: 230 - protein, 353 aa, Bifidobacterium infantis
MPDTMVIPEVLADEAELRATHVWRAYHIFYGGNPLIVLRECILPLAEDLQREGVILDYFFINYWLEGSHVRLRLRVRPERVEELDARVLERVRQYLAASPSYHPMAELADNNFYESLFAGEFTDADRPKYFDANGDPMFAENNSIEVREYEPEWLRYGGEVGMLISERQFVESTQLMVRLMHLGNLGVRTILLGIASQISFITAVCLLQDADLVEDFFVAYHHRWADGYDTNPAYGTEEGRRKHQVTVENLRKKIVPRADAIRRGDVGDLPQILRDWVQVCLKVRFQIEQACEHSALRFKYDDGVRSVTNVDDAAWSLCHSYIHMTNNRMMVSVADEAFLAYQLVEAMRGSDD

SEQ ID NO: 231 - protein, 359 aa, Bifidobacterium infantis
MTKSVIDWVPRLNPLLRVGPPRWNGTAIVTDIGIDGEERLIEIGLAERCVLDEVDGSRDVSTIASELSAKGMPVDTTRIVGVLNRFAYVGAVERPFSMKAGLADIDWAANEGQRVRPDQIAGAADSGAGLGLWRKLTFLAHPVVFGILVVAGIAGAAFLAINLSDALATVLDATVWQAILAGVAAVVWTGAVTMLHESGHGALFHHESTRSPFLALTRFGLILMPNTHMPGFSLLGARERARVLAAGPLVSMVFAALPVVLFVTVDEPQVRIIAAMCMCCDALIIALGISPFPNTDATRLIEAWVGVDQLQAVAFRTLVGKYSLPAGLPLRSKVAIRLYPVLLLDTIALWLAVIVCILN

SEQ ID NO: 232 - protein, 62 aa, Bifidobacterium infantis
MNLQNLAAELDSFTAETFEVKDYVDYADMAFGSTCSSSSSSSSSCSSTCTSCCTSTSSCATA

SEQ ID NO: 233 - protein, 279 aa, Bifidobacterium breve
MARNANKNQVYEKLSEALNWCNAHNDNAQQRFNNCVNNVIDANFHAERNQEMKSLFPVLLLNNLSGYRPGITMKETTLDLHSPDFLDLYFTAWAQKYFKAWETLPSHRIAKPKEAATDPALIKMVEPQAGNIYIARDWAAYHNLFMSAENVGGNLLEEYIYTKVHDYGWTWCRGEVLTAVDFCSMDKERFIQIKNKSNTENSSGKGFREDHNADKWYRMEAKKKNGLVVTRWPELIQIIQEGAPDGVTVPDDLMTENSYLEFVRDAAARNRQLITDKEL

SEQ ID NO: 234 - protein, 433 aa, Bifidobacterium breve
MGVSPSVITRLSNTGKLDCISVGARKAFKQSTVDSYLAQHNQEHAAADHCRKSTELPKIVALSFFTGAGGLDLGMEAAGIHSLLYCENNRECRMTINHNRPDAALIGDINQYEALDILRLANIPESRDVDVMFGGPPCQAFSTAGARRAFDDARGNVFLRFLKLAADIKPRYLVIENVRGLLSTPFPTEEGGKPVRGGAMRVILNKLDDMGYAVSFNLYNAANFGAAQIRERVVIIAKREGQPAAWLTPTNSSDAAWGLPQWNTFREVTKNIEQGSQHYIQFPEKRLKFFRMLKAGEYWTSLPKTAQREAMGKALDLGGGKTGFYRRLAWDRPSPTLVTSPIMKATDLCHPDELRPLSIEEYHVIQGFPDDWWIAGELSDIYRQIGNAVPVKLGEAIGQALINDMNGIPNKSDWDDFPYSRYTHTSNKTWNLA

SEQ ID NO: 235 - protein, 479 aa, Bifidobacterium breve
MAQRTNNGNVDNGIVIRFEKLSRQSIGNITKIVGMVRAKYLIPIIDRLDLQANPRNSKTGTVTKEIQRSIENDPLLFPFKTKGLLLASSHYEEMERGRIRVTFDDRTTEGILDGGHNTLAIGLLILDRSMGYARAAGLADVKMPAGAKTWADFKQLWKKNRDLIQKYQESVRKSENSQPELGTQDEDLSFYVPVELLVPTDPDNPICAEEFSNNLLEICVARNNNAELTTGAKANQHGYFDDLADALNKVDPNVAERIEWKTNDGGDIKVQDFIALTWLVLKWLCPVTDSEGKKVEAPSMRKLYSGKGSCLALFERFMSSEDITSQDPSSYRHGLRNPDVYRAFELAAQIPALYDRIYKDFPDLYNQAGGSYGRITAVKSLNARTTNKTTPFSNMPIDTVSPDGYITPLVCGLSTLVDAKTMKWKTDPNKFLDRWLPSIVKRYMQVFVPCDYDPQKVGKSAASYSAVEDAYKMAFANIL

SEQ ID NO: 236 - protein, 49 aa, Bifidobacterium breve
MTEENKTPQAATEEQLLSATAVDGQTITLTPISLEEAFKVFDGKEDNSE

SEQ ID NO: 237 - protein, 119 aa, Bifidobacterium breve
MVGDNVRLNDVPLFRDESLDWHKMFGSLTVSIYHRFGSIYEVSVIGTVSGGSHYNYTTITDQLPHKFESRSVPGFMHSSYDSAGVVFEVIDGRVLQLLGKSVSQDMSYDVRGMFVRVYA

SEQ ID NO: 238 - protein, 282 aa, Bifidobacterium breve
MANDNALTWGELTEKKTMDDQGVAVDIRGVNKRFVTQQGDHVTALHDINLTINKHDFICVVGRSGCGKTTLLNMLAGFEKPSDGELISGGVAINGPSPKRGVVFQKPPLYPWLTVRKNVEFGMKMQGVPATERKEKADHFLDLVGLTSAADRRPYELSGGMQQRAQIARVLATEPDLILMDEPYGALDALTREKLQNELLRIWRERHSTVFFITHSVDEAIFLATRVIVMSAHPGTVKMDIPITLPRDPDDPDNMEKVRAMPEFVKLREEITQAIYVQDDQE

SEQ ID NO: 239 - protein, 365 aa, Bifidobacterium breve
MKILRETTRETVRKIGALLVSATVLTSLAGCGKMPTADELVGNTSGAAVADCPVPSAKNEDFKGTIRIAWQAIPNADLIVKDKGLLEACLPNAIIQWSQFNSGGDVIQAFGSNSLDIGLAGSSPAVRAASAPLNLPVKVIWIHDIIGDAESLVARGGDYKSIKDLAGKNIAVPFGSTSHFSLLSALHNAGMNETSVNLINLDPDKMSAAWTRGEIDAAWVWDPVLSKLKADGGTIVTSSAATAKTGSATYDMELATDAFIKANPKVMETWTAVENYAAGLISSKEADSAESISTILGNSVADVKKQFKGYTYPQAKDQSDIFHGQLPGIFKDTAEFLKTQGSLDKVSDDYSSVLYTDAIDQVAKQ

SEQ ID NO: 240 - protein, 311 aa, Bifidobacterium breve
MSEATTPTSNASNASDTPVVAGTAVAGTSIVEGAATTQRKPMPAWMKSKKIQPIISLVLLIAVWWGVTSAGLINSLYLPGPKAVWDAFIEANSCRPASAGSSRIVCGEQQYFLWQHLVVSLERIGIGMALGIVVGVLVGFILAEIGWLNRIILPYINFIRALPPLGYIGLLIVWFGIGDTTKIWLLFLAAFPPIVLATVDGINGVNRDRVNSALSLGASRPKAFLFVVFPSALPSIMNGIRLAVGFAWTTVVAAELNNGIPGIGALAYLSGTELNTPLTIACIFVIGIAALLLDWLILGITHLVTPWVGKE

SEQ ID NO: 241 - protein, 545 aa, Bifidobacterium breve
MGKTHSTKSASVDGSSSVAVRPFGWRDKIGYMCGDFGTDFMFVFAGTWFMIFYTKVMGIPGTIVGTLFLLARVLDAVMDVTVGVVVDRSKDHPGGKFRVTMMRFTAPLVILSFLMYQTFAIDAAMWVKIVYMSVTYVVWCFFYSCVNIPFGSLASTISAEADDRTSLSTMRSMGGTIGGLILGIVAPIVIYQKVDGHQVIRGGDGTHIFVIVAAVVSIAAFIGYLICYFNVTERVHPDPDQNAKEGEKRSPIHMIANAFSSRSMLGIVAAALCLLVAQLFSNQLMTYVYTDVFGSAALASISGLLGTAVMFLVVPFVKPLTRRFGRKSICSFASALGAISMLLLFVIQTRSGIVFLVGAVFMYFALMGFNLVIWAMITDVIDDIEVTRDTREEATCFSCYSFARKIGQAVAGELAGLSLDWVGYQSGATAVQSSATKTALYNVATLVPGVFFLLTFVCLVIIYPLSRDRVLYNVEVLRVKRKGITGEEARLLVKYEGITPTGTTPEGSTIEWPDFHELERARELSARTGIAIRDPQAEASTRIEQ

SEQ ID NO: 242 - protein, 563 aa, Bifidobacterium breve
MATIYTGGTIRTMTAPDATAEALLTDDRGRIAFVGSLDEARNRAASMITVGGRVPEEHDLAGACLMPGFIDSHSHFAGMGQRLTDADLAGCADFAEIGTRLKTFLAAHPLPAGGVLHGFNYDQNELAEGVHPDRYVLDAMLGDVPTVISHVSGHVIICNTALLQLIGLDLDADAPAGGVYGKGADGKLNGYFAETPAIMPILMHPAVMPSQSVEDLADAIQNDYASHGITACQDGATAPGYAERFVKLAENHQLKLDLTCYPMMGQDLDAVFESVGSYASGGTSEGIQYLNHLRFGGVKLFVDGSPQARGAWLSEPYEPLPASAKVHEPAGYRGAGILSDEAMRAFLDHALERGWKVMAHCNGDAASEQFLTQYTAAYRASSKPDKFDLRPVMVHCQLTRNDQYARMAAVHMIPSIFVSHCWFWGDAHIKNVGFARASRISAVHDALDYHLPFTFHTDSPIVPPNLIFAAWCAMTRVTKHGVELDQSQCVGAWEAFSAITRNAAYQYGEESRKGTLEAGKLADLTILDADPLAASADKAEAAAHVRELKVLETIKEGTTVWTA

SEQ ID NO: 243 - protein, 376 aa, Bifidobacterium breve
MLWDYQQPVAIRFGNGRVREIKDVAAEMGLTEGGLLVSEHLFAKNGTAEKIVKDSEGAISEIFSDFSPNPDVTEVDKAAALIRDKHLKFVVAMGGGSAMDLAKSAASIALTNDSIAEYHGTGKAMPQEHLPIIAVPTTAGTGSEVTCVSVLTNRALGKKAPIVSDGFFPSVAIIDPELTYSVPPHVTASTGMDVLSQAIEGYWSKGHQPICDACAIHAAPLVFKYLPIAVAEPDNAEARQKMCEASVIAGLAFTLPKTTSSHACSFPLTNIHGIPHGEACGLTLDWFARINKDAQHGRVQEFARAIGFENVDAMADAIHELKVKVGLRTGLKDLNLNAEQIADLVRISRHPNLYNNPVEITDEMLQDMYEHLAATD

SEQ ID NO: 244 - protein, 453 aa, Bifidobacterium breve
MSESLATKELTGEEVAELHRKYVMQSWHKQGEPVTPVKSAKGIYVYDYDGNKYADMSSLLVCSNLGHELPEIVDAIKDQASKMCFMAPAYASEPKSKLAKMIVDLAGNDFYQRVFFTNGGADSNENAIKMARMVTGRQKIFSCYRSYHGSTLGASFASGDWRRFATEAGGAAPSFVHFMNPNMYEDGFNRGEDDDKVTALYLKRLEDQLIYENPDDVAAILMESIVGANGVILPPKGYMEGVRALCDKYGILMICDEVMAGWCRTGKMFAWQNFDIKPDIFTFAKGVTCGYVPLGGVVVSKQISDYFTDHVLQCGLTYSGHTLACAAGVAAVNYYVEHDIAGHVKEMEGILKPFLEEMEAKHKCVGESRCIGLFSAMTIVKNKETRELMSPYHTANPVMGKIMGALKDKGFLTFGRETNVNICPPLTITAEELKAELPKVDEVLTWVDDNFCD

SEQ ID NO: 245 - protein, 392 aa, Bifidobacterium breve
MTVRLKELVEQAADKNMVLAAGKNGLDKPVRWVHMVENEEIAGFLEGQEIAFTTGIGLETQEDLYPLVKSAYASGATGVVVNIGPFIHQISPETIRFCDEHDFPLFKVPWSVHMAQIMHSFSLAITMSEKHSMELAAALENAIFHPDREEMYLEYLEQSGFGKDWNYCVAVFSACADAEHGGHDAELIAKYSREAESLITANQWRVALVRIEDRLVLVFARYTAEQVELMVREIIAAIRRRGVILERTYIGVGKVTKSARCIGKSYNQALKLERLQHLRGRVGEVALYDNSGIDKLLLAVSDRAILEDYYQDSIGPLVEYDRVNGTDLTETLHAYFQFSGSVKETAVSMFVHRNTVSYKLNKIEDILGVSLSDFRTREFLSVGLQVREILDC

SEQ ID NO: 246 - protein, 239 aa, Bifidobacterium breve
MGESVRDMLFVVVLFAANVIQAITGFAGTVLAMPFSMLLLGTDTAKVVLNITTLLVCLWLGVQHRAHIRWRILGEMVGLMAIGMVAGVALYAALPLAPLQKAYGVFIIVIALKNLIWPAHAEPPHWLLIIIVLLAGVIHGMFISGGALLVIYAAVRLKDKNEFRATMACVWVALNSVLTVQQGVAGVITPHALALTAVSIPPLIVAIIIGNKLQKRVSQQAFLKLTYVLLVISGASIVL

SEQ ID NO: 247 - protein, 66 aa, Bifidobacterium breve
MTCATFPGMRHGLGLLPGLAVRSIDPACYFTLTVRNGVNRLIHIRKIDKRHCTVLRRAGGDMLVEA

SEQ ID NO: 248 - protein, 61 aa, Bifidobacterium breve
MLPRSHPAGNLPQAASGIESLAVDACGRPFPARWPCQTSVRRHHFCQPMSCGRLPAKPDGP

SEQ ID NO: 249 - protein, 123 aa, Bifidobacterium breve
MTAPKVAASPQDIAILNLNMLMQLEGRYRKDLAEYIGRRPQNLSRMMSGESNWALNDMWKAAEFVGVSLDVLTDPTLTPAKALSIIGERRNDNDGNGGLPVVNVDDFRLRGGAWKAQAMVLAA

SEQ ID NO: 250 - protein, 227 aa, Bifidobacterium breve
MGASVDVATWVSVGCAAVSAVFAGVTVWWPWHTRPAPDLRHEKDEFSVTRESMAHLLVTCGLQRPRLLVRWRNDGDGTAYAVTIKAADGSCAVRMAVPDTSKPSGFDFVDSVGKMEPGESFEAIILPTSTEDVGKPVVLLDWKESPTRLRMGHRSERVALPYRLPGKRPLLRQERILALHMIRTTAAEYGYPYEQFASQMLGIDLEDLDPWSAPDSKGKTESPDSGE

SEQ ID NO: 251 - protein, 315 aa, Bifidobacterium breve
MPEPPPKPARRGAKTVGHQGGADLVVVVTAGHLGHGLDVADILGHKHEHDGQEHRQDGEVGLRQVELREADPCGVINGGEVDLAAEAGINVADDHADQNIESTKQSLEQHGDKQHGQQSHDCGIRRGLEIGPYGGRQIEADNGHDRTVDHRRHDDVDPLGTGVMHEHTDQSQQDTGAHDAEAGDRDALVGGGDCGDRGDEAEGRAQIAWQHVLVDEQEQCGGHGGKEQGGGRIKARENRHQEGGAEHGDDVLRADAGGTRPSQTFIRLDHLAGLQRLAVAMQLPLEDIGHYYSPRSRRQARHTHTSDRAVRHCIS

SEQ ID NO: 252 - protein, 60 aa, Bifidobacterium breve
MDRATDEHSQQERKPATSCLARLSGATVRRMPSADRFAYAAATVLMEYPMAIMQDMASSA

SEQ ID NO: 253 - protein, 143 aa, Bifidobacterium breve
MIGFSPSLSVEHASASMPLANAESGLVPIVHATGMPSWQSITGDGQALPAGIENSVRSVTHGMFGRSAWKSPSTRFAGALVSSPLYEPCRFALLDRGHQAVPGHEPHDLLRAGHDSHAPQLQVDPLVPVPALAVLERLAHELQ

SEQ ID NO: 254 - protein, 115 aa, Bifidobacterium breve
MRRSPATTTCPETERPARASQTAGKVATYNWTDKDGQGGCYGGNLYNEIKGIWERADRNEKALAQLTALVAAQQTALDTLAKSLGANPADIAEIVAQAVTAKLDSIDVTFTATSK

SEQ ID NO: 255 - protein, 238 aa, Bifidobacterium breve
MVQIKEEIVNQGHGYLSPSLFAVHSTANPGATARNHRDLWSRGYDYAVHLTSDWKEAIHCVPYDRLCWQVGNGNTTCEGIEICEATNASDFWKGIDIAADVIAQRLRAHGWGVDRMHPHSWFSQTYGGSDHTDPIPYFSRYGYTWPAFIQLVQQKLSGNTGTQEVDIMAAMMIRNDSTGVIWYCEPGKGRTALTHPDQANLLQQAGVPLIHGNNGAPWWSRFDQIDSMVRGTMKKLGV

SEQ ID NO: 256 - protein, 118 aa, Bifidobacterium breve
MNLPEGLPAWAYIVVSALVLAAQIVTAIWINHRGDERDRATRGEITNNHEMPLRDDLDDKALRTLDAIESLRGAVDGLRDDMNGEFATVNRRITTTEQNLIELRHEVNDLRRGGNNHQ

SEQ ID NO: 257 - protein, 114 aa, Bifidobacterium breve
MKYSAKRNAHGSTPSYVDRDSLTPPLHLYTGSGVYKDAGSGGYATMWTFGQFKTQFGRDWNNDVVVAAINGDWDANGRQVTSVRVTPSGNRIDVMFDGKNTAPIRVNWAVMWRG

SEQ ID NO: 258 - protein, 68 aa, Bifidobacterium breve
MAYVKKTNLRGPQGPAGPQASTEQIFDKAWPIGTVLETNSDSTPPTYNTGWKKLPNIMGRGFLWQRIG

SEQ ID NO: 259 - protein, 216 aa, Bifidobacterium breve
MAYVKKLNLTGPQGPQGPKGATGPQGPRGPQGPQGAKGDAFAIAKTYKSVAEMNAGYATDGVLNGQFVMIDTGNVNDADNAKLYVKGAKGYTYITDLSGATGMTGPQGPQGPKGETGAQGEKGATGAKGDTGPQGPQGVKGATGAAGAKGETGPAGPAGPTGATGPAGPQGEKGATGAKGADGNTITYGTSAPAAGATGKAGDMYIQSNGDLYIFE

SEQ ID NO: 260 - protein, 279 aa, Bifidobacterium breve
MSIQLIDKIKQKNDGNFYLVDAVDVEYNKKSLIDALKAGDIIPAGGGTSAGTFHVANINIGSNTAFDSSKLTPSTVSAGDLIVDANGAFYTVAKVDGTTVTPSAALTADGGGTLGFKGAKGDKGEPGAQGPAGPQGPKGDAFTIAKTYASVAEMNADYRNADIPIGSFVVISTTDVNDADNAKMFVKNDTQYAFVTDLSGAQGIQGPAGPQGIQGPKGESGEQGEKGANGTPGAKGETGQRGNRITVGDGDPGEPPADALAGDVYINQANGDFYQVQDQ

SEQ ID NO: 261 - protein, 240 aa, Bifidobacterium breve
MANAWKKMGSLRGPAGAGADVATSEKAGVVKPSGDFDITADGTLSLYTPMSVMSFTGGSDHEIGETVDTVNLAWKLNKTPATLTLDGQEIVKGEDGQFPTSQPLTKQALKANKTYTLAVTDARGSKASKTTSVLFHYKRYWGVGGNPADGVDSTFLLALAGSELGDSKAKTFTVNAAAGQYIWYAIPHSFGTPTFKVGGFEGGFNLVKTFDHTNASGATVSYDVWQSTNAGLGNTTVNAA

SEQ ID NO: 262 - protein, 104 aa, Bifidobacterium breve
MIRTLNGCNCDDLVTIIDDAIVMEGQKGESAYETAVRLGYQDTEAEWVDSLHGKGITLGHGDPTSQLTAREGDGYLNADNGDLCEYTSEPDGSEETNDKESNHG

SEQ ID NO: 263 - protein, 61 aa, Bifidobacterium breve
MTSQAIRDKVLAWHGRGYGATDTARQLGLPLEEVRAIIREGDGRPKPPCKVEFIEPPLFEE

SEQ ID NO: 264 - protein, 79 aa, Bifidobacterium breve
MADETEPAMFDALEKALMPLNSARQLAELSGIGESTLAEWRGTHTGPAYVKSGRRVLYPKEAVLGFMRANLRECKEASA

SEQ ID NO: 265 - protein, 66 aa, Bifidobacterium breve
MAQWNIRFNDELIGPFDDAETQAISQKLTTSTRTQGGVVFSGKLADSGNDVTAYWTPGCPISFEQI

SEQ ID NO: 266 - protein, 190 aa, Bifidobacterium breve
MFQMPRKATTLFCMVLGQIDLGQILVTALVSAVVSFAVTVILRVWDRRTVEWLVAGEAHPTYFAGEKKDMVLNLELWNTGDADAYDVRLIRCNGVNPDTGKERECWETFEAGCIKAGEHVVFTMKPSFASWETCWVKVVFRPSPVYRHNPRCSRKYLLSKEIGNQFEYRPEESEMAVYGRGKIPARPKDA

SEQ ID NO: 267 - protein, 123 aa, Bifidobacterium breve
MGEARFQHVRKTPDIGEMHNLYSRDYDNQDKLQNVDEILATHFAPPSASAGIAMLGDMRSLAATAKCLAICSISSKVKGDLFSKRRLSVDKGIFVAPASASMLSRREPSSARIFDTASLLRLK

SEQ ID NO: 268 - protein, 344 aa, Bifidobacterium breve
MNDHPKLTKEQQDTCERLLDKSQEAFILAIELFNRPTIRYRVEGCAFFLCNAWELMLKAYIAKRDGYEAIFYPGKENRTLALEDCLKKVMTNDKDPVRLNVESINELRNTGTHFVVEEYEITYGPIFQANIRNYDDRLRTYHGIEICNRIPDNYLVLSVNRTDIDGESIRAKYTPEVAERLLSMQNDIDLRSAEESNTKYAAYFRTEFVLSKKEGIPIRVDNSAGSTARVIKQVVQPDDRYPYRMSDLLKLVNRQLLRQAVKFTSGENADARFNNYHFNLFVKCYGMKHDERYAFDRATAAEKKAGRHQYTYSNAVVNFIVEEIAKDPEHIVEKLRRKVEGEER

SEQ ID NO: 269 - protein, 73 aa, Bifidobacterium bifidum
MGMMPTMMRRWASWTMGWALRMSPVSHGLQESGKGRAHTARMWEAAWLKRIRMGCAVVVPGAAVMLLSGPTRL

SEQ ID NO: 270 - protein, 59 aa, Bifidobacterium bifidum
MPPTTVADTAPSTAPGNRASAPSTVPAVKIAIAMMNRVFVANLRTMNGELGIDTDSNSR

SEQ ID NO: 271 - protein, 69 aa, Bifidobacterium bifidum
MQFYSRPGKILHDTTKKLPNHQQRSSIAVSFLATPSGPAASQHVVVLLLFILGGIPICQESRVAKVVPF

SEQ ID NO: 272 - protein, 108 aa, Bifidobacterium bifidum
MNIIISGLKINTAKGELDASPLAHKSGILNLTLAENSANELIAGAFHAGLEKNDVNGAPDETFALNIDSQGDGADAGWGSLTAKSLYGTIGKSSYSAGIGGAAVYRRH

SEQ ID NO: 273 - protein, 437 aa, Bifidobacterium bifidum
MEDRISMATKRQITLRFKDEYAKASKKDKGVILDRMCETLKIGRSTARRRLKEAGRAGEGREAPRERPKRYSDRSRLLLEQVWLLMDLPCAKYMKEMLPQWIPTLLEAGELRGFDTATVDELLAMSAATMDRYLKPVRDAANPKGMASTRPAGELLRNSITIRKAGDELDGLPGNVEADTVAHCGPSLKGEFCRTLTVVDFATGWTENASARNNAYRNLSKAEAVIEERLPFAIRSYDNDNGSEFINLDFISHLQAIDVEQTRSRPYRKNDQATVESRNNHIVRRHAFHYRYEPAELDLLNELWELVRIKANLFTPSKKPVGRASTRDGRPKRVYDQPMTPWERLKRFDDEDRARGGPGFIKPGRRERIERLIAETNPAELVRRIHAIQDELEQAAMPRTKRLERKLGPDMAYLNKTLARITGADVSETDDEPAGAS

SEQ ID NO: 274 - protein, 436 aa, Bifidobacterium bifidum
MNRKTNMPRYTLTLIDSTNQTPPIVLTAESTRTIIRQLTKRLDPTPERRRVQGSGGFTMERGRVKAFRELPPDPATGNRRRATAFGATRAEAERRLNEKIAELQRVGRLGYTKPPTVAEWCDHWLNDICKPHLKPRTWGTYASVIANNIVPSIGAVRLDELKPAHFRRMERYVMDEQGKSSGTAGSAWRTLHKALEDAVLEGVIERNPAVKGTAPRVALKERAALTPEQAADLIAAETDDTWRLMWMLAFMTGMRQGERLGLTEDELRRDGDRLIILVEWQAQHLTKREVEGLPKGVECTPLGNGMYRTRPKTEKGRRAIPLPEALADQLLAYIERHGVNGDGLVFHDDRGLPLTGCVERRRWYRALDRVGLGHEYVPHSARHTTATILNRLGLDDVTRTAIMGHSRVSTTNEIYTHVELDRLVAATDGVERAIEG

SEQ ID NO: 275 - protein, 84 aa, Bifidobacterium bifidum
MPGNAGPVWSLPLLTVPGRMRSGMEVPELMTATQVARILGVSTETLRKWRARRMCLPYVRVGRHIRYRAADVAAFIERGTVMPA

SEQ ID NO: 276 - protein, 124 aa, Bifidobacterium bifidum
MPYIDIHNERVWIPERPLRSMDEARPMRDGDLWRCPACGRELDEEENDDGTITLVCPAHQVGWDAGHEDEPYPMGGLVFRPTMGGGVFIAGPCMAVCLDRTSAVNAFFALGETYGLTRIEGVRP

SEQ ID NO: 277 - protein, 74 aa, Bifidobacterium bifidum
MNAFKGRGGHPHCPACWARMFRSAPLDGGPATLVCLRCGLERPLEPVAGPPEPYTRALRALDMLADALDEDVMP

SEQ ID NO: 278 - protein, 126 aa, Bifidobacterium bifidum
MNRLRGFRLRAGMTQAELARAIGTTQSHVSEYESGARRIETMPAGEFVRLKAVPDVTDALLLDADLSGIDPDAVAAAENRVARLRRRLRRRRDRASFDLLVEAELDLARRMDEREEAVARAMGSRA

SEQ ID NO: 279 - protein, 62 aa, Bifidobacterium bifidum
MDTDGLPKEQLLVDRPTAMRMLGVRDPRTVNKLVRNGRITGFTACGRWYFNRASIEAFARGE

SEQ ID NO: 280 - protein, 436 aa, Bifidobacterium bifidum
MAKAWIRDRWLKSKAVVDGVELTPTTGMKRQVAANPDTADVPAALRTADYGRGMRWQVCWRADGRQHRESLPTREAAEARAAELNDDIRSGRYVDPRGGNRTLDEVFPLWLADHTGVRPATIDNYIRHYNGMVRPRFGSTRIGDIDERAIKAWVADLDSGAIRTKYGEPYTKGAIKTGVRRLLGSMLRYAVRRRWIMADPTAGVRLPRAPMQRVDAFTPAEARAIADAAGKLRTPTGRPCGRPMDRLIVLLLASTGMRPGEMAALDVRDVDLAHGTINVDKTMTKAEAGHYRYVQGDTKTPKGRRRLPIPPFLRDGLEELVAGRDGGEPLFTSPRGERLLYAQWSKRVFRPAMAAAGIDPDGRALTLYSLRHTFASVAIAAGADVKTLQELMGHEDATVTLNTYAAAFADRRVEVAGAVSDAFDDALGQTPTDGHF

SEQ ID NO: 281 - protein, 838 aa, Bifidobacterium bifidum
MPNHVDPEDAMRPGKDYTAEQLNGLLALLNSKYDLRGGDIDTHPVLGGIRFSEPPALDTADRWLTDSGPIAEAAKTSTGRPIWPIGYQKAVTAIVEGRYRCCEDDPDTFYALDRPDDGVEAMSSYHVLDFAVELEVRTSGRVREIERALKAMIRLMPRLRPDLYAPVRRGARIHTPSGRQAVMYVKDGRPTLVRAQDGSDGVPYIVDVEVPEEALPGSDKDWRIMCGRVHQWMRFVTKTEASAMNLALYWAYPIMAPRMENLWCFYGDGGNGKGMLMKSFKDVFGRWCGDIDIERLAEGGFDGGNEAGKLTDALWVFDPESDMSDAKCARAMKRIATGDPFTARYGGGVAYEVRPHCGFMAATNLPPYKEATRAFERRLVEVNAYDGHLPAEFAPMADWLDNRHGAVELLLTSANLWAGGYKLDLDESIGTLTGLGEDELFAIKELVEKGFTVSADNPYLGKGRRYSRSFINRTGLKTGRRQGQFGLMVKSESMFAPYREAMESMLTADVRRAEERRADRAANPATDLSTLAPNGMDPVDWARAKRIGGDTGVYVPAEGKVARDWKRKTENGEATPLPDFTKSDSAAEVAGEGMVIIDWDADHDGTGDPHGLNRMECDLGAVIGSDDFPMPYLERSARGGYHGAYRVPSDLMPFFKKSAVGKATSGDPTLLVDLKAAGGGYVICAGSRTDQGAYEPVSAPADGKVPMLTQPMLELFSEYGFIRLPEAYMGPDGKPHVGHGDARPTVAPSAYARFKSSDGRLHLDTSPMAPHTRHNELVRRAKYVVAASRAMDLPESAVTEAFDTLRALAGDHDPSDTERVLRDMANGYGCKYFEPRND

SEQ ID NO: 282 - protein, 66 aa, Bifidobacterium bifidum
MDGQDELVTIAWIAKRMSIARPSVTKIMRAMGVPRYEFTSKCVRYPKSAVEAAIAASYRPASAGNE

SEQ ID NO: 283 - protein, 213 aa, Bifidobacterium bifidum
MRRGRTSPSAPNALRRPEKAANTLNRGSLTASLQLTMAFAACREWLHSAVFRPEMQLMQLLQLFAYKKTERHPDDTWGNHRPPCLHRKEIANDYINYINYIYCLKMASFRRFRAFPLTAIRTAATAFLQLTSSSRSRGAQIAFTLVDFGHAMKAEDRCPSCPPVCLRQPLTSQSGIYPLHRRSRMIPPFSPSDSLRTHRRRLKVFAASGRIDA

SEQ ID NO: 284 - protein, 387 aa, Bifidobacterium bifidum
MSLPPQRQLDNYLSQFAEKQVDGKYLGLYDGERRFGRVFAWLHEQYNGAFEFMNAKAPQGVGGHFNADPSRDLMEVNETYSALLRIASKAGIRIKTKPEYQKVIDSSRGWLQPTLGSPIPEGLTPIEVEYYDTVFETEDSGIMLAGTNRVPLQFVGEGSYAIVHKFTDPNYGIQFARKKLKKGVKPKEVERFHREFDIMKRFDFPYILKVYRYNESDNSYTMEYCEHTLKDYISHNNQKMSPWARRKMAMQFLYAMNFLHKHGVCHRDLSYGNVLIHTYDDGAFAVKVSDFGLAKERNSDLTSMGSSMKGSIEDPALKSFKDFKPVNDIYSIGFILNYIFTGRRDLLADGSRLGSIIQKCSTTNSADRYQTVKGIIEDMRKTECPVG

SEQ ID NO: 285 - protein, 299 aa, Bifidobacterium bifidum
MEQEALLAQARKTFHTRLIDWGVLGYTVKKGEPVASNADVSQTVSKAIGNGIMTRVLNESQQEPRKPLSGQALGAQFEIAVCDFIADTFPHLGHLRPGEWIIKRLGNRNAVTEKNFAQYEHLAALGRAVEQDPVLQAALGNDYVVAPDIIVARQPETDQTINAPFAVVNDVVANLSDLRQRHEGESQRAPILHASISAKWTMRSDRAQNSRTEALNLIRNRKGQTPHIVVVTGEPAPSRLASLALGTGDIDCVYHAFLPELIASVDELKGREDLAELIHTLVDGKRLKDIADLPLDLAI

SEQ ID NO: 286 - protein, 400 aa, Bifidobacterium bifidum
MDALVGLEEVAGLIGAEPRIRQNSPSHDDLEAKASTDLSQRVIRLAKRLEIVVFSPLCRRFEVTLRYMTKYTDVELCAGAGGQAHGLELAGFEHVALVEIDRHACETLRLNRPQWNVLEADLHEWDPSAYEGVDLLSGGVPCPPFSVAGKQLGELDDRELFGRALEIIRQIKPRGVMLENVRGLLDPKFDTFRKNVSDRLRRYGYSPQWRLLQASDYGVSQLRPRVICVALRPIDMRHFVWPEPDEATPPTVGELLGDLMAANGWKGAAAWSKQANTIAPTLVGGSKLHGGADLGPTRAKRAWMKLGIDGRSIADDNPPADAPNDFTPRLTVRMAARIQGFDDEWQFSGRKTSSYRQVGNAFPPAVARRVGQQIIRAWEATDGTGSAISTGKEDIPHPTD

SEQ ID NO: 287 - protein, 403 aa, Bifidobacterium bifidum
MTNPQAPFQTEQPATQHTEALVRAEGMLGTVELHGGSLITIDHEGVAALAAKTRHVEIPVTAIVDVVLTRPTIMTNGRLDLVVRTTDGDSRYDPKSPNPFSVSFLAKDLDGFRNVAQSIKAAKPAEPAAIEPPKPEKRRPFWRRGAFWSMAALAVVFCCLVNSCGSVSDDSATESMPDVVGMDYAHVHQALADYHDVDYIDLKGNPTLSGVATKQSPKAGAKVSHSTGITVTLDPQGKTKEQKEQELSKRVADCKDKDAATVIAGLDADKLTGTIKTSPNAPTDMADTIRGGIAQGTAYIVTDAIVDNGKIDLVVDTVAHHNRELASQQIPAMCEAAGKQSHPYGFKVPLFSDNSGVVFSDDTNWSYSFEANITNAFGAVAKGVPITCTGTLTGQNIVVSGIN

SEQ ID NO: 288 - protein, 282 aa, Bifidobacterium bifidum
MAGCSAWFRYAVCHHGASFRHGSAGENMAVFVSHAFVLYYSGALFRYEKYDSILACVKDQAKKLLFPYAMLTIVCAPFWYINHIVLGDGKVSFKRLLVGFLTADQYEGTMTNGALWFLPALFLTSVVFWFLVDLEHKGKVNLAGSLGICAALAIGVGIFCEGKPTAWNVAGVPAMVIFYGIGCMAMQSYRRHRDAIESLSPTVMTAAIIALMSVGTWAALANTKVSLLVNDYEVFALMLVSSLGISACLALLFMDLPNVRPLTFAGRFSGLSWSAYSYDAFL

SEQ ID NO: 289 - protein, 113 aa, Bifidobacterium bifidum
MRGLAAALGLVHQLVGVAVVAGAVRYVFVLITHDVPDSFGRCGLMVTYAMYQAKLEECYSLALPKQLRDAVQQTAILLSEGKSVTISATEQELTTEQAAYTGSIATDASQNVR

SEQ ID NO: 290 - protein, 97 aa, Bifidobacterium bifidum
MFADALEYVTAQDQPENEIVKNMKHFFQKVGDNYLKFTDPNSKCIILPNTDENQQIIKTLCNWKPQLIPQIEKIDEKNLIKIRRDQKSWNEFFKQGE

SEQ ID NO: 291 - protein, 571 aa, Bifidobacterium bifidum
MALIADTTEDNEDSLRVGLNANNASDASTPQAWRELTGCEETDQVYVEYGSRWARTTRLIPYSLLKRVIDTSLDIGQWEETQKTILTNKNADLSKTIEDLRSGDWAFILQFEESQTKESQTSLGKDFYALLGGNTSLLYRLISHGWIGQDYMLYSTIYTDEGITRNALNFILHHIDGNNPDYQFKLDDKDSEDVLRRIRRKDKAIFHQHRVLNIDLLHYMLSHCDDSETNQDTDQIIEMLSPLGNETAEFVSAYLDRLADDANPLLVKMASHSQNLLSFLAQRGTTGTDMLCQHLDVAFHHLSREIQYTVDGNLADFLQNHWRSISIFTDSNIAPAFVEPIVQILNDGKVRIDSLSELGYKDSNSGLRYPCVEQNLYVFSRGNLETALDTISQSDNNQDGSYSVCNRLPALNMIMHISRTVYANVLGHLEEYFDLLTDNEKTLDCPSGQNIADDLFHILDDICQQAGQEKSAEKLIHTLLDKCSPSARFPLFEDRFDHFEDRFDHFDVYATLMENDAVCFTPINLQFVFNRGFDEGNHSLVEWIQKHDSFEQDSERKEITPEFLRTFGFRP

SEQ ID NO: 292 - protein, 475 aa, Bifidobacterium bifidum
MTKKSKADSTDNSTNTTDPTSKAAELKPLYPQYDESSHSPYVKRLEKALQGEDTKIHNIALSGVYGSGKSSILEKVVKDLEKERPHTTRTISLAPLAAQLKKQDDRKVNSISNEGNSASREAPGALPPKSSKPSSITNLIQKEIIKQLLYGTDPEKIPASHFHRINEIGLGKQLLSSLACGTLLLFILDIHKWPYNRIQELLTWLPIPTSITKILAEIVIIGLLLIATFALFHYFGTRIHLAKIDVGTAGITLGENSDSYFDQYLDEIIYIFEKTGIRTVFFEDLDRFQDAQIFDSLRELNQILNNDPKLQRTKSTIQKNSRQSHSSHSVKEKNSQSSTPIQFVYAIHDAIFSNQYVSAAEQVSNKESLAHSFSRAKFFDLIIPVVPFVSASNSCQIASETLEDVLDANDPQMINLLELVADAVPDKRTWINIRNEFIVYREHLFVQSPEGRFTSKLGLEESHLLAFIIYKKLLS

SEQ ID NO: 293 - protein, 1058 aa, Bifidobacterium bifidum
MRKSVEPAGVPTLPEVGSIVEVRGATWAVTDVRGQGISRSPADESRGNRQHVVSLQSLEEDRMGEELSVIWELEVGQTVLPDQGLPDIINADSFDDPNRLGAFVDAMRWGAVTSADAKKFQAPFRTGANLEPYQLEPLRRALSAPRTNLLLADDVGLGKTIEAGMVVEELLLRHRARTAIIVCPPSLCVKWREELLEKFGLDFVIVNSETLAESRRKYGLGANPFRLYPRVIVSMAWVPSIRAQRLLEEAYSDTGGMNSARQYSFDILVVDEAHHVAPAAPASSGRVRGYAVDSKRTETIRRLAEHCEHRLFLSATPHNGYSESFTALLEMVDSRRFTRGAAIDEKALEEVTVRRLKTNIASLHFKERHIETIPFTPGDDEEQHYAMLVKILAKTAKEHSDRDRALGVTALLFKKRLLSSPWAFARTLSRYRDLSEDDGYDSWMDDDYYSEVMGSGQSDEEEGREEQPEFETLAKGRKDNPLAAAEEGQLDELEDWADRFEGRPNSRLEALIEWLDSVCRPGGGNVWSDERVVVFTEYADTLDWIQRILESRGYTKDCLSVIDGQTDAEERELIRARFNANPKDEPIRVLLATDAAGEGIDLQTYCHRLVNFDVPFNPSRLEQRIGRIDRYGQTQTPEIYYFRPSQKGSLLEGNLEFMNRLAQKVSVEVEDLKTVNPLIDREISDHFLGGNGKPQVRAADEMGRQANEIINKTLAGSVALNRQLTELADEYETSKRMMHLTEQAEKRVVDVSLDLTNQPPLITTDDPKVYRLPDLNPGWRPIEDGLRTVLDPDRVRPITFDADVARHNPDVAYMHLGSALMSKASRTLRGNLYGQESKLHRVTAVVVPGLDATCAAAMSRLVLVGRGGLRVHEEMFVTGLRFRAQNLAEEKVQTLLADALDASKTLQLADPKILERLEREWDANNGRLRSRLEDAIGKRAETRKQLVESNLEERRESDLDRAKGIFEQFRRNLRDSLAALRRQDEDDAVQLSLWEDEEQKQRKRDIRKMEQRLDDLDSEESRELDMIRLRYKDVKPYVSIAALVFAVSEQDAEQWRAE

SEQ ID NO: 294 - protein, 911 aa, Bifidobacterium bifidum
MMVRNRRSNQRMRSNSPEQMHREWLELVDTDGPFLAAPVLKRAWPQGMSLLKTLENGQQRAAELRQEKAAFEAAWDEWHRVRVGSDDDEAVADATRKYREQENAWVTFVVRRLLDWREDYRLAEDDAAEAAAYDAESPNGAIRVSPSGLLELNGTVGAVVLVVDPVVESLTEIPDDGWNASAIDRMQHMLRTKQSTCSIGLVTDGRWWALVSAPKGKSAAWGQFDSQMWIDTPQVLNAFVNLLSIRSLVTDAEEDRLPALFAESVTAAEDITEALGGQVRQAVELIVAAFNESSARARNAGRPDPLPDDGERSYEAAVTVMMRVVFLLFAQERGLLPRSGLFENAYGLAGMLDMLEERARDEGEEAMDGTSMVWHRLLATSQALYGGVNFEDMRLPAYGGSVFDPQRAPFLVATDEHGELTVVVSDRVMHHVLRSVQIAVVGRESRRISFRDIDVEQIGYIYEGLLGYTCLRSKEVVLGLEGAKSQEPEVPLSVLEQIAADSADDASGAKRAKAIEEWVKANASASKTPSRSKMGKLLTGSSPEDVERALLSVTHDKAMQDRLRPWMGLIRRDLRGKPVVFLAGDLYVTETSSRKDAGAHYTPRALAEEVVVHALEPLIYQPGPLQTNDRSQWRHISSTDLLNLHVADIACGSGAFLVAAARYLSAELVEAWRLEHALPYDGTPEQTRLKAIRMVVARCLYGVDINEMAVEMCKLSLWLVSLDKNQPFSFVDNKILHGNSLLGVTNLAQVEYKRIDAKPRQQIQLFEINDLNQTASVVDVSPVIRRVRNIREQLSSEISADDPQRSAVAKHRQMHEMDEALAQLRKIADGVVAAGLRGICESGKRMDEEYGNLAVAVGRAFPAEGEGDSRMLDDIIDSGLKPTVPTDYEHWRCVHWDNARILAHFRIPALT

SEQ ID NO: 295 - protein, 495 aa, Bifidobacterium bifidum
MIQSAQLFGHYLHWPLEIPEVMENGGFDAIIGNPPFLGGKKLTGTMGENVRKWYINILAGGNRGSADLCAYFYLRSYSLMRKGGTLGLLATNTIAQGDTREVGLDSMTDNGFTIVRSVQSKPWPVSSANLEYAAVWGVKGAVSDDVAKDCDGMKVSRISTLLEPQGRASGQPDALKDNKDVSFIGCYVLGKGFIISQEQAAEWIEADLKNKEVLFPYLNGEDLNSRPDCSASRWVIDFNDWNEEKAKKYALPYKHLLKYVKPERQRKKPDGSYQLRKSLPERWWQYGDKRPALRKAIFPMNDVLVIALVSKILMPLRVPTGQIFSHALGVFATSSYADQAVLSSSIHQYWAISRGSSLESRLRYTPSDVFETFPRPQNTDALAAIGKTLDEERREIMMRRQLGLTKLYNLVNDPDISDSSDADVARLREIHRHLDETVMAAYGWSDVPLDHGFYEYRKMIRWTVCPEARIEILDRLLEENHRRAKLEAAQGGEHE

SEQ ID NO: 296 - protein, 1247 aa, Bifidobacterium bifidum
MSEQTTETANPESKIYELGYPLDGSSYACRENLQDILRREMMGPSNGENEILEVSPKSKYILGRIAPTKIMDADEIAHHGMRENTDADPNEEPDEDLDDDAESFDDELHDAPQRRGLLIPSSMGMRFQIPDDLESFTVHCSWGRYSPVGTGKQDKRGNEVRAYQRTPVAHSVKVRLSDLVNDRTTIRVEDTVELCVDRYDDPALHRCFIEVALCNDTESEKPIPVSKWLFQTQLDVDADGKAVFLPVHDWNEDPSFEKEQDFEQKKLRLQYRNRLEFAVGRTCSVDWTVSPENSRRAVSVRTTWLPTADIPQTIAQNVEGAELDMTALAVMGPEELRKALTPISDGYRSWLDEQEASIAALPEHLRKTARGTVQLARIVSKQLADGIDFLCSDEEALRCFHFMNTVMAEQRVHTQVNALRGTNGDLSLKETEKRVLSGSYPHHWRVFQLAFILMQIHALTDPAVEVRSDESTKAKTQLLFFPTGGGKTEAYLGLAAYTFAIRRRQGIVKSPDGNLNGNEGVAVLMRYTLRLLTSQQFQRASTLICAAELERRKNPELWGDEPFRIGLWVGTNVTPKKVSEAAREIEQTRLRASNRNPDVLQIVSCPWCGKPLGSGDLDVDKVRGRVFVHCPDMRGECPFAKGGEVTEGIPVLTTDEEIYRLVPSFVIATVDKFARLAREGAASALFGYVGRKCDRHGYVPNLDIEENSDYDDCSIKDDSAHPEKRNCPAAHIHPAMRLRPPDLIIQDELHLISGALGTTVGLFESAIDVMCTWKDQNGRDIRPMIVASSATMRNAADQIRKLYGRGVTVFPPQVLDVSDTFFSKEQQCDEDTPGRRYIGISTTGVRLSNAEIQTAETLLKGAQRLMNDPKGGNAADPYMTLVGYFSTIRELAGMARFMQDDISTHVRRGRLGSHLPRRYGAQFGDLNVGELTSRISSTDIVTTLDHMNNTFDEDHDSQQAWHRNAELRKQGKGTNNRDSADIPFDAVLATSMLQVGVDVSRLGLMMIVGQPKNTAEYIQASSRVGRDAKRPGLVVTIGNWARPRDLAHFEQFKAYHDSFYARVEPLSVTPFSVTALEHGVEGLLVSAVRVMQANQKTGLNPERNAGLAAQEHGVLDTIVTRLIERIHIAGGDDAAKEADSRLRNRMDTWENLARSASGNGRTLVYERAPKDDSDYQRLIHSAEELGNREALTSRCFVIANSMREVQPEINILVSPNPEKLGFTEPDGAPTWQSQQATNTAKQETEHE

SEQ ID NO: 297 - protein, 687 aa, Bifidobacterium bifidum
MSDTKASEDGLIYDPQLDVDPLGDQDELEENNTAKNYAKVGSSRGSTLMYTYGPGSIMDLPHFTVMPMGLNAWDKIWKRRPGISKITAPRLLENVQLMLGNQVKELRPFPWQPNQTGAFREGADLGVPARMFPQWLRCTGCNKLAPVSDFVNGYSNTNPYRPDQAEFMHKGCHGSGKGAKKYDRPCVPARYLLVCEDGHVDEFPYDWWAHNGGHCPNASKPQLKMIESSAGVSGSFIECVSCGAKRSMLEAQSLENRSKLPRCRGRFAHLDCFQAKPCDKPVRLMLIGASNLWFPVVQSIIDMPRLDEKAVIRDEYNMIKTALGDNDWMLEEDLDDNLKSIRKTVQRSAKTDDELKQKSEVELHSIILQGQQSEMNEDERQLAREQWEPSDLLVPEWKYLVRDFPDTKHVDRKSGLTVHVQQVHGIVADLGVKRILAVDKLKKVNALIGFTRVDDFDRVNDLGSRLVRLNRDGRPTWVPATEDYGEGIFIQFDEERIEKWENAVLANPLWDSHVMAHRRNFRNRLSETAAVVDPDTRLPKPRYWLMHTLSHALIKRMAMSAGYGIASLSERIYAWQGSDDRPAAAGVLVETTASGSDGTLGGLVDLSNTDKFEQIMTSALQEMKRCSSDPVCARRIPKDPEDFLHGAACHCCCMLSETSCERANRFLDRRFLVPLPGQDTSLAFFKD

SEQ ID NO: 298 - protein, 263 aa, Bifidobacterium bifidum
MDKIQTDALIELGASMTGLEASDIANYLEDGYPLEEAFSNVQEFHRKRICELFEIAGFGDDDEQQVQETIKELRGIQGAYRDPEQTTAVWTSPHGLVREGDLNSSRSHMIEAATSSIVCSTFNFQRSSALWESLKKAATLPGMSVKIYVDTSANSADDTTRKGKGPTSPTPEEIAKEIVGAKVFCTAKSDKGYYYRNHAKFISVDHQDLLVTSANFSYSAEELNIELGLRIHDEALAESIERQMANMESRLYRRVGVREEPDE

SEQ ID NO: 299 - protein, 424 aa, Bifidobacterium bifidum
MNDSDFNPRKRHVVFQKLNITPSSMPIISLIASIKNVRANGLDLSPDYQRGYIWSNEYKDQLILSIILNYPIGNIVINNLDQPNQRNARQELVDGKQRLTTIFRFMEVGNVGQWLDSYDDWFQLSKKTSDQAKEIINRIVGDSDPDGLARMHRAKRLAFSDLPSSIQMNFNTYNIPVYTMQAADPAQIRNYFKVLQNQEKLRAGEIINALPDNPMSMYFDRIPAEAFLTRTGCSNFKRAELEKVYYSVLGTWFDKIQINASDKTVISFVENMPELTEAQIEHINNLNSGIIAISRLPGAVQKIRSSKRMLKLVFGLALHAPGYFSTTDAFSRLQSVCELSSKLAAFNTSDSDQVAFSKYFGDEYTLDKENFETRKACVYRALFWSTSRVSSRTAYVDAMEILRRMFTESFDSAFEYYTAHNIAK

SEQ ID NO: 300 - protein, 30 aa, synthetic construct
  REGION 1..30: note = Synthetic: recombinantly produced peptide, GLP-1
HAEGTFTSDVSSYLEGQAAKEFIAWLVKGR